SEQUENCE LISTING (94 sequences)

SEQ ID NO: 1 - DNA, 202001 bp, Homo sapiens
  source 1..202001: mol_type = genomic DNA
gcccagcaggtgtcagcctcattttaccccgcccctattcaagatgaagttgttctggttccaacgcctctgacatattagctgcatcattttacatttcttttttttttttccttttaaatggggtcttgctctgtcacccaggctggagtgctgtggtatgatctcggctcactgcaatctccacctccgaggttccagcgattctcttgcctcagcctcccgagtagctgggactacaggcacccaccatcatactgggctaatttttgtgtttttagtagagatggggtttccccatgttgcccaggctgatctcaaactcctgggcttaagcaatacagccgcgttggcctcccaaagtgttgggattacaagcatgagctaccccacccagctcattttacatttccacttgttaaactgaaaactggcccgagaaagcttctgtactgccatccttgcgtccttgcagatgaatcgtaacctagcatagtaggtaggcagactgaaaacctaacttagcagtaggcttctgtaacaacagctgtgtctcagccagttcctgcagccagacttcaaccactcacaggccgcaaactgttcaaactgtgttcggagaaggcgaattcatctggctgttaacgtgcctcacttctgctttctgtggccactttcccttttctgtccataaatttgctttgaccacacagcatccctagagtctccctgaatctgctgtgattctgggacctgcaccatttgtgaattgttttttttttccttgatcagctaaactctgttcaattcaatttgttggaagtttttaacataccaatggtgcaccaaggttccaatttctccacttcctcataaataagtcattttaaatggcttttcagtattccaatatttggaagtattaatgtttctaccaattttctatttttggacattgaggttgtttcattttttttttctttttttgagacagagtctcgctccgtcacccaggctggagtgcagtggcctgatcccggcccactgcaacctccacctccctcctcagcctcctgagtagctgggattacaggtgcatgcaccaccacacccagctaatttttgtatttttagtagagatggggtttcaccatgttggtcaggctggtctcaaactcctgacctcaggtggtccacctgccttggcctcccaaaatgctgggattacaggcctgagccactgcgcctggcctcatcttcttgatattaatgttgctttaacatctttgtccctgtgttttttgtttttttttttgagacggagtctcattcattctgtcacccaggctggagttcagtggcgtgatctcagctcactgcaacctctgtctcctgggttccagtgattctcctgcgtcggtctcctgagtagctgtgttcctgggtctttcgatggttatttaatacttccctacagtaatgccctgtgcgtacatgctaagtgtgatgaaatggttggcacagttaaatcttttgaaagacattgccaagtcactcttcagaaaagtgataggaggtcatagcaattttaagaagtcctcatttctacatttccttactaatctcggttggtgtctcttcaatctttcctcacacttttcttgggtttttcctgaatcatgagtctactacatttacacattttaaagcatctttagaaacaggatctcattttgttgcccaggctagagtttggtggcatgattatagctcctcatactcctgggctcaagtgatccttccacctctgaaaccccaaaatttgagaaaggtctcatttaatttagaaagtttattttgccaaggttgagggtgcacacctgtgatgatatacgagttaaaaagaaattatttaggcagatactgagggtaagaaagtcctcggtaaggttttcttttcaatgaaaagcagcccccaagcattttcttttctaacaaagagcagcctgtaaaatcgagctgcagacatacacaagcaagctggaagcttgcacaggtgaatgctggcagctgtgccaataagaaaaggctacctggggccaggcagatccaacatggcggctccatcttccctttccttgtcaaccatgtgcacagtaaggagcaggcaacatagtgtcccccgagtagagaccaatttgcataataaaaggtgagggtagggtgggcagcttctttgcatgctatgtaaacattatgcctggtccaaccaatctttgggccctgtgtaaattagacaccacctcctcaagcctgtctataaaaccctgtccattctgccgcaggctggaagacccactggggcacccctctctctctataggagacagctattcatttttctctttctttcacctattaaagctccactcttaaccccactccgtgtgtatctatgttcttgatttccttggcatgaggcaatgaaccttgggtattaccccagaaccttgggtattatgccacttcagtgacacagcctcaggaaatcctgatgacatgttcccaagatggtcggggcacagcttggttttatacattttagggagacatgagacgtcaattcatatatgtaagaagtacattggttccgtccagaaaggcggggacaacttgaggcagggagagagcttctaggtcacaggtagacaaatggttgcattcttttgaatctccgataagcctttccaaaggaggcaatcagaatatgcgtctattgactgggcgcagtggctcatgcctgtaatgccagcactttgggaggcggaggtgggtggatcacctgaggtcaggagtttgagagcagcccggccaacatggtgaaaccctgtctctactaaaaatacaaaaaattagctgggcgtggtggcgggcgcctgtaatcccagctactcgggaggctgaggcaggagaatagcttgaacccagaaggaagaggttgcagtgagctgagatggtgccattgcactccagcctgggcaacaagagtgaaactccatctcagaaaaaaaaaaaaaaggcctgggcaaagtggctcacgcctgtaatcccagcactttgggaagccgaggcgggcaggtcacaaagtcaggagattgagaccatcctggctaacatgatgaaaccccatctctactaaaaaatacaaaaaactagctgggtgtggtggcgagcacctgtagtcccagctactcggcaggctgaggcaggagaatggcgtgaaccggggaggcggagcttgcagtgagccgagatcacaccactgcactccagcccggacgacagggcaagactctatctcaaattaaaaaaaaaaaaaaaaaaaaaaaaaaagagagagagaatatgcatctatctcagtgagcagaaggatgactttgaatggaatgggagcagttcctagcttgaacttcccctttagcttcagtgatttgggggctcaaggtatgttcctttcacatacctcagcctcccaagtagctgggaccacaagtgcatgccaccacacgtggctaatgttttattttttttgtaggaatagggtctcactatgtgtccaggctggtctaaaacccctgagctcaaatggtcctcccgcctcagcctcccgaaatgctgggattacaggcatgagccagcatgcccggcctagtctacatttttataaattgctaattcaaagttccctctccaaaacctcatggttttccctgttctcatcccctgcaccctcccttcccctggagtactcacctggccttggaggtctggtgtgagcccggacttcgattctaggcacagcatgtgatgagcgcccccaggtcaaacacctcccctctgcggcctgtgcttcaccgccttgacagtgagaaaggtctcccttcggctcattctcgaagtctcaaacttcacttctcctgtgcgctgattctgaattcagcccccgtccaaggtcctggcccctttctcttctgcttggcgtgttgttcatcaccactgtgcactgctgagggtaagtgcggttctctggacctctgctttatcattagaacagactcttgcggtttcccacgacattcctttcacttctcacttggaagatgagccgtgaggaaatcctgtgttgtgtggtatgtgggctgtgcttctgcttgacttgagggccaagcagcattgcaagccatggttttaaataagaaagaacatttctaaccttcatcttctagtaaggaaacaagtgggctttagagttcttgctcaggaaagacctatgtcccagtccaaccggaccttttactaaagagatcttcctgatcctcctccccaggccaggggaggggtcctccctggggttggagcctttagtagggggtcggagacacgacgtagccttcatgacattcatagtctagttacacgatccctgtaagggtcagttgaagtaagtgctacaaaggaagggaggtgctcagtggagagggctctcttttatgtattatatttctttcatggggagggatatggatcagggatcagcagaggtgtttcagtcccgagggaaagaaagtcagcgtggcttgggagttgggagcagcaagacagtggctcaagatatcttaagactagtggagtacaccttgcatgttaaaagccttgctcagggctgcctggttcttgtaggacgacagagatggcctagctctgcatactgcacccccaggggctcagaacagtgcaaatgtcagtctatctgtcagtggcagagccagccttggagcaggggtgcaaggaggtctctgcactggccaggcatgcagaacattctgttcagtagcactggacagaaggccccatctagatgagacagagctggtggggcaggacaaagactcctggcagctcaaacggcctggcagatgcttggagagagggggcttcttgagacagcaccatttctgggaagagagtcacctgggagggatgaggccacgctccggcttggaggtgaagagaggggctgctgcaagaaagaattagagacatgccagcctttgctgtgttgcccaggctggtcatgaactcttggcctcaagcaatcttcccacctcagcctccccaagcgctgggattatagacatgagcccccatgctggccaataaaagatgattttatggaggggatggtggtgaaggttgtgggtggtatgaaatagtaagaaatatatattggtctgcacccagttcctgccacagagctcctaaaatcctgagaacttcctgggtgagcatcttttgttctaatgaggtgactcttggtggctcctggataggagtgaatcaccagaaagatcaagccagagttagaagcagaaagtgctggctataacacaggaaagctgtaacacaaataataaagtttttttttttttttttgagatggagcctcactctgttgcccaggctggagtgcaatggtgcaatctcagctcactacaagctctgcctcccaggttcaagtgattctcctgcctcagcctcctgagcagttgggactacaggtgtgtgccaccacatctggctaatttttgtatttttagcagagacggggtttcaccatattaaccaggctggcctcaaactccttaccttgtgatccgcctgcctcagcctcccaaagtgctgggattacaggcatgagccaccgtgcctggccaaaagacattgttcttaaaagaatcaactaactaaccaaataaataaaaatctaacctaattaagaaactaaaaatacacaaaaattaatttcaaggggagaaaaatcatgtaaagagagaaagataatgaatactttgcagaaatttatgaacataaacataaaacttggatgaaatgcatttctaggaaaacataatttatcaaaactaaccacaagtaaaatagaagcctaaataggatattttcaagagaagaagtaaagttgtcaaagtgctacccttcaaaaaaacaccaggctcaaacaatctgacatgggaatgttagcacaccttagagagcaaataaaactttgaatgggcttgaaatattccagactctagaaaaacaaaacttcccaattctttttataaagcaagtataaattgataccaaaatcttataaagaccttatacaaaacttcataccaatctcttttatgaatacaaaacccttaataaagtattaccagacagaacccaacaatacataaaaatgtcacatcataacatagtggggtttatttcaataatgcatggatggttcaatacaaggaaattcagtaacacaatataatagatcatgtgaatatacccaaagaaaaaatagattattttcatagatgctgtaaaggcatttgaccaaattcaacacctactttttaggtggtcaataaaataaattagttactccttctttagcatgataaaatatatttatcagcccagaaggcatcattttacccgataagggcacacgctggagggaataatgttaaaattaggaataagaggatagctagtttctttcttcttttttttttttgagacggagtcttgctctgttgccaggctggagtgcagtggtgcaatgttggctcactgcacgccccccgcctcccaggttcaagcgattctcctgcctcagcctcccgagtagctgggactacaggcgcgcaccaccatgcccggctaatttttttttgtattttagtagagatggggtttcaccatgttggtcaggctggtcttgaactcccaacctcacgtactgggattaccggtgtgagccaccacgccagcccaactactttcaacattatccttaatactgatgcttattgacttactatggggttacctctagataaatccataataagttgaaaatataagtaaaaaatgcccttaatacacctaacctaccaaacatcatagctgagcccagcctgccttagctatgctcagacactgacgtcagcctacaattggcaaaatcacacagcagcacagtctactgcagagcatctgctgtttgcccttgtgactgcgtggctgcctgggagcttcccagcttcacaagacagtattacgtagcacatcactagcctggggaaagatcaaagttgaaaatttgaagtgtggtttccattgaatgtgtactgcttttgcaccatcatcaagtcaaaaaattttagttgaaccagcctaagtttgggaccatctttattttcaggaggaacttccatgtacattgatgacggacgatagaatccgtttctatcatcctaatgaacataatgaataaatccagacaaacataaacattaacagagtaagcagctttcggggctggaagccagaagagggtgggagcgcagagagagaggccaaacaccagggctgcttctgctttgcgggtatttgctgatctggacaaggtatctggaaggctgagctaagcctcctttttttttgaggtggcgtctcactctgttgccaggctggagtgcaatggtgcgatctcagctcactgcaacctccacctccctggttcaagcgattctcctgcctcagcctcccgagtagctgggattacaggctcccgccactacacccagctgatttttgtaattttagtagagacggggtttcaccatgttggccaggatggtctcgatctcttgacgtcatgatctgtccacctcggcctcccaaagtgctgggattataggcgtgacccaccgtgccccgtctgagctaagcctcttgagcataggggactaaaaatgaaatctagcgcatgccaagtttagggtcccaggcaattcctttccactttggggtccactttggggtccaccccacccaagaagaaggatgacttggaagtaaaccagctctgaaatatggatggtcctctgggaccataccaatcccttcatatcaaccacatccagttcctcaaaactggaacttggattaagatggcctaggacttctagtgtcccaggagcctggcattgcaaacaaaaatcctctccggaagaagataataccttaagcttcaaatgactctctaataaatttcaaatacaatgtccagcacacaaacacaaattaccaggaacgtgatatgaggcctgatggatgggaattagcagaaacttcaggcatgagaaacataccctcagaggcctagaatctatctagtgtctagataatggagatatgaaatacagacacttaaacaactatgtttcccatgttcaaagaggaaatttgcaaaacttgaaagtgttggcaggaaatcagaaactataaaatgtgacaacagcatactttagagtcagtataaattacggtcccgaaaactgcagaattccagaacttaatggtaaagcaagggtttaacagcagaatagaaatagccagagagaactaggaagtaagtcagatgacactacccagaataaggcactgagaggccaaggaatggaaaatgcagaagaaaggatatggtgagaggatctaatatacatttatttggagtaccagggagagagagaaggagaagaacagaagccgtgtttcaaggacggtgactgagaggcttcgaaactgatgaaagccatcagttcacaaattcaaagcccagtgaattccaaggagaaaaaaagaaatccatactgtgaaagcaagtccagacaatgacaaacaccatcaacaatacacaggacaggcataagatgcatttaatggggacactcagaggcagagggttatcagaaggaggcacttctctcccaagttctcatcatcccagggccagggacagctggtcacaccttagggagttcactaggagagggatctggcttcttgtcattctgggtatttgtagggaaattggaagggaaccgagagcacctagccaatcgcatagcaatgggagatttcaggctgtggggaatgtctttgctggtgaaaagaacatcctgaccttagaaatctttcaccgagggggatctgcgttccagaacttctggagctggtataggtaaggctttgagctttcctactgagccagcctgttgctaggttaccaaaggggacctcgagggccatctggccaacaagcagacttgtctctccttacacccccagacgtatcactgcaaaactacagaaaaccaaagacagagaaaatcttaaaagcagccagatttaaaaaatggcatattagtttcaaagcagcagccatgaaattgacagctgatgtctcaacagcaagaatgaaaagtggaagacaggccaggtgtggtggctcaggcctgtaatcccagcactttgggaggccgaggcgggtggatcacgaggtcaggagaccaagaccatcctggctaacatggtgaaaccccgtctctactaaaaatacaaaaaaattagtcgggcatggtggtgggtgcctgtagtcccagctactcgggaggctgaggcaggagaatggcgtgaacccgggaggcggagcttgcagtgagccgagattgtgccactgcactccagcctgggtgacagagcaagactctgtctcaaaaaaaaaaaaaaaaaaaaaaaaaaaagggtgacgaagcttcaatctcctgaaaggaagcaactgccgcctttgattcgatacccaccaaaatccgtgaagaaggaaggcaaaataaaaacacttcctgattgaactggaaagatttccgcaatagaagacccactgtccaaggaattctaaaggatgctttccaggcagaagaaaatgaccccagaggaagatcagagattcaggaaagaaatggagagtgataaaaatggaaaattcgggggccaatttaaacaaaagctgactgctctacaactgttgtgtctctatcttttgtaacatatatgtgtgtgtagcttttttttttttttttgtcaagatggattctcactctgtcgcccaggctacagtgaaatggcacggtctcggctcactgcaacctctgccccttgggctcaaatgattctcttgcctcagcctcctgagtagctgagattacaggtgcctggcacaatgcctggctaatttttgtatttttactagagatgggatttctccatgttggccaggctggtcttgaacacctgacctcaggtgatccacctgcctgggcctcccaaagtgctaggattacaggcgcgagccactgcatctggcctatgtgtgtgtttatatggaattaaaacacatggcaataataccctccaaattgggagaaaccaaaaatagcatttaaatgttgtaagctccctgcataatcaagaagagaatagatttacgttagattttgatacctggaggatgaatgttgtaatttctagggtgaccatgaaaagaggagacaacggtgtatgtttttttttttttgagatggagtctcactttgtcacccaggctggagtgttgtggtgtgatcttggctcactgcaacctcctcctcttgggttcaggccatcctcccacctaggcctccagagtaggtgggatcacaggcacctgccaccacacctggctaatttttttttttttttaaatatttagtagagatggggtttcaccatgttggccaggctggtcttgaactcctgacctcaggcgatctgcctacctctgcctctcaaagtgctgggattacaggtgtgagccatcgcgcccggccaacagtgatcactttcaaactaacagaggttcaaaaataaaatcagacttaaccaaaaaccaggtaacagagctggtaggatatacagaaagactgacctcacgtatatcaacgattacagttaatattaatgaaggaaatgctctagtttaaaaacgagggttgtcaaagaccccacataagaagctccttaccagcggtgcacctagaacctaaggaaacaggacagatgaaggaggacgcgcccccgccgctgtcctgcgcctcagccatcctatgagacgggaaaggtttctgtctgcagctgggcccgtgctctttaccagctcctggctttcttctctggaaggttcctgcctgttttgccctcacacctgctcctctctcagccctctcaggggtggggctggaggccaccaaagagcctcctctgctctccagttgctcgactgctcctcatttccccctggggtctgcgtcagggtttccttcttttccagccccaccccgcgtgcatcccacctggtctcgggtcggggctgctcccgcttactgccccctgcccaggctggtgtgcaccccctctggctgctttcaaggcctcttctctcttctcggcaggacaggcacaggcaggtggccaggtgtcatgcttagctccccgcccagtgagattctttcatttaacaatcttcccctgaatagttcatgttcattgctgaaaatttgaaaaatatggaaaagcacaaagattaagatataaaccgccctcaattcccctgcccagagagagtcactgctatgacttggtgactaggaaccttatttctctctcgctcttttttttttttttgagacagagtcttgctctgtcacccaggctggagtgcagtggctcgatctcagctcactgcaacctccgcctcctgggttcaagcgattctcctgcctcagcctcttgagtagctgggattacaggcacctgccaccatgcccggctaatttttgtatttttagttgagagagggtttcatcttgttggtcaggcggacttgaactcctgacctcaggtgatcagcccacctcggcctcccaaagtgctgggattacaggtgtgagccactgcgccttcatctctcttctgtgtatgtgtacgctgttttttctttagaatgggggacgttatcaggctctacatggtgtgtagtcggctagcatgttgtaagcctttccctgtgtcacaagtgctcatctggaacaggattctaatgactgcctgtggctatgttgggattcctttaactcagctccttctgcccagcatctatcttttttccatcttttgtcctaagtgttgctataataaatcattgatcacacatgcctgactgtttgcataggataaattacgggaaatgtttttgctgttcagggactgtgcccatttttaggcctcagagacaccatgccagactgcccagtattgatctttactctttttagatgatgccaaacttttctgtgaactttaaaaacctgtgtcttgacagtccatttctgtaagtctttcacattagatttcctgtcaggatgatagtcaattctaggcagatgatgttttctcagccatggctgaagcagttgtgatttgttgtggccatgtaaagtcccgatgatccattgcctccctggatgggttggaataatttggtttgggagcatataacagaatgacctggagtcacagcagctcagacggaagtgtatttctcccttacagatgaaagaattccaggccaggctggaatgacaactgcacacagtcatctgggccccctccttccagctcccatcaccccaggatgtggcttttatgcagatgatccaaaatggctgctcaagtcccagccaacacatcccattccagggagcaggaaaaaggtgtgtctttcccttcattttatgtgattcctttctagaagtactactcattacttctgcttgcatctccctggctagcacttacttagttatatggccatagctagctgaaggaaggacagggactgtcatacactagctaagaggcaaactgcttagataaaaaggtctctaaagaaggtcagagcggctgctagggtgcaactctattacttattgttatgggacgaactgtgtccctcattcaggttgatgtcctaagccccagaacctcagaatgggattgtatttggagacaggttctttaaggaggtaaggaggctaaaatgagatcattagggtgggccataatccgactgatgtcttacaagaagagattaggacacggacatgctcagagggacggccacgtgaggacaccaagaaaggcagctgtctgcaagtcaaggacagggctcaggggaaaccaaccttgccaacaccttcatctcggacttctagcctctaggaccatgagaagatacatttctgttgtttaagctgcccggtctgtggtactttgttatggcagcccaagtaaacaaatacagtcatctgctgctggaacaaatcaccccagcactgtggcttggcagcacacatgtctagtcatagagttatatgtagttacgtgtagagccatatgtatcgtcacacgttctgtgggtcaggaatttggacccagcttaaccagctccacttctcgccagggttcagtcaaataccagctgcctcccacctgagagctcagccggggaagggtccctttccaatctcacgtggtgttggcaggatccagttcctcatggcctgctggactgagaacctcagttctcactgcctgttggccagaggccgcctttatgtcctcgccatgtgggcctctccaacatggcagctgacttcatcagagcatccatgccaagaaggcaacagagagggccagggagactgaagtcatacccttttgcgacctagtcatggggtgacattccatcacctttgcccattggttagaagcaggccaccaggtacagcccaagctcacggggaggggtcatacaagggtgtcaataccaggaggtgaggggtgctggggccatcttatgagtctgcccactgaggtaactaacaaccttgaggcctgacacagtggacaaaggcccttattaacagcagagaactgggaactttatttatttatttatttttgagacagagtctcactcttgtcacccaggctggagtgcaatggcatgatcttggctcactgcaacctccacctcccaggttcaagcaattctgcctcagcctccggaatagctgggactacaggcatgcaccactacacccggctaatttttgtatttttagtagagacagggtttcgccatgttggccaggctggtctcgaactcctgacctctggtgatctgcctgccttggcctcccaaagtgctgggattacaggcgtgagccaccgcacctcgctggaacttaatttttttagagacagtgtcgctctatcacccaagctggagtgcagtggtgcaatcctagctcacttgcagcctcaaattcctgggttcaggtgatcctcccacatcagcctcccaagaactgggaactaacagctgtttctctgctgtccttctcaagaaaagggaggctactgctaccccactggggacaatgctgggtttccctttaggacaggctctgagacaaggcggaggtgctgtttgtggccacagagcaggggactctgggttgcaggtgtggcctggctaaagtaggctttactgggctcctctctgcctgcatcaccccccggctgggcggttgtctctgaggccaaccttactccctgctgggcaggctggacagctgccctctccgtttgcccctctaccacccaaaaggcaggaggctctggagaccaggaccctgcccgccacggcctgtgtcccaggcgtgagggggtgccccacagacctctgctgagctgctgctgaatgacgccccttgggggtcctgccggaaggtcagagcaggggtgcactcccataaagaaacgcccccaggtcgggactcattcctgtgggcggcatcttgtggccatagctgcttctcgctgcactaatcacagtgcctctgtgggcagcaggcgctgaccacccaggcctgccccagaccctctcctcccttccggggcgctgcgctgggaccgatggggggcgccaggcctgtggacaccgccctgcaggggcctctccagctcactgggggtggggtgggggtcacacttggggtcctcaggtcgtgccgaccacgcgcattctctgcgctctgcgcaggagctcgcccaccctctccccgtgcagagagccccgcagctggctccccgcagggctgtccgggtgagtatggctctggccacgggccagtgtggcgggagggcaaaccccaaggccacctcggctcagagtccacggccggctgtcgccccgctccaggcgtcggcgggggatcctttccgcatgggcctgcgcccgcgctcggcgccccctccacggccccgccccgtccatggccccgtccttcatgggcgagcccctccatggccctgcccctccgcgccccacccctccctcgccccacctctcaccttcctgccccgcccccagcctccccacccctcaccggccagtcccctcccctatcccgctccgcccctcagccgccccgcccctcagccggcctgcctaatgtccccgtccccagcatcgccccgccccgcccccgtctcgccccgcccctcaggcggcctccctgctgtgccccgccccggcctcgccacgcccctacctcaccacgccccccgcatcgccacgccccccgcatcgccacgcctcccttaccatgcagtcccgccccgtcccttcctcgtcccgcctcgccgcgacacttcacacacagcttcgcctcaccccattacagtctcaccacgccccgtcccctctccgttgagccccgcgccttcgcccgggtggggcgctgcgctgtcagcggccttgctgtgtgaggcagaacctgcgggggcaggggcgggctggttccctggccagccattggcagagtccgcaggctagggctgtcaatcatgctggccggcgtggccccgcctccgccggcgcggccccgcctccgccggcgcagcgtctgggacgcaaggcgccgtgggggctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccgtgagtttgggcccgctgcagctccctgtcccggcgggtcccaggctacggcggggatggcggtaaccctgcagcctgcgggccggcgacacgaacccccggccccgcagagacagagtgacccagcaacccagagcccatgagggacacccgccccctcctggggcgaggccttcccccacttcagccccgctccctcacttgggtcttcccttgtcctctcgcgaggggaggcagagccttgttggggcctgtcctgaattcaccgaggggagtcacggcctcagccctctcgcccttcgcaggatgcgaagagttggggcgagaacttgtttctttttatttgcgagaaaccagggcgggggttcttttaactgcgttgtgaagagaacttggaggagccgagatttgctcagtgccacttccctcttctagtctgagagggaagagggctgggggcgcgggacacttcgagaggaggcggggtttggagctggagagatgtgggggcagtggatgacataatgcttttaggacgcctcggcgggagtggcggggcagggggggggcggggagtgagggcgcgtccaatgggagatttcttttcctagtggcacttaaaacagcctgagatttgaggctcttcctacattgtcaggacatttcatttagttcatgatcacggtggtagtaacacgattttaagcaccacctaagagatctgctcatctaagcctaagttggtctgcaggcgtttgaatgagttgtggttgccaagtaaagtggtgaacttacgtggtgattaatgaaattatcttaaatattaggaagagttgattgaagttttttgcctatgtgtgttgggaataaaaccaacacgttgctgatggggaggttaattgccgagggatgaatgaggtgtacattttaccagtattccagtcaggcttgccagaatacggggggtccgcagactccgtgggcatctcagatgtgccagtgaaagggtttctgtttgcttcattgctgacagcttgttactttttggaagctaggggtttctgttgcttgttcttggggagaatttttgaaacaggaaaagagagaccattaaaacatctagcggaaccccaggactttccctggaagtctgtgtgtcgagtgtacagtaggagttaggaagtactctggtgcagttcaggcctttctcttacctctcagtattctatttccgatctggatgtgtcccagatggcatttggtaagaatatctctgttaagactgattaatttttagtaatatttcttgttctttgtttctgttatgatccttgtctcgtcttcaaagtttaattagaaaatgattcggagagcagtgttagcttatttgttggaataaaatttaggaataaattattctaaaggatggaaaaactttttggatatttggagaaattttaaaacaatttggcttatctcttcagtaagtaatttctcatccagaaatttactgtagtgcttttctaggaggtaggtgtcataaaagttcacacattgcatgtatcttgtgtaaacactaaacagggctcctgatgggaaggaagacctttctgctgggctgcttcagacacttgatcattctaaaaatatgccttctctttcttatgctgatttgacagaacctgcatttgcttatcttcaaaatatgggtatcaagaaatttcctttgctgccttgacaaaggagatagattttgtttcattactttaaggtaatatatgattaccttatttaaaaaatttaatcaggactggcaaggtggcttacacctttaatccgagcactttgggaggcctaggtggacgaatcacctgaggtcaggagtttgagaccagcctggctaacatggtgaaaccctgtctctactaaaaatacaaaaattagctggtcatggtggcacgtgcctgtaatccaagctacctgggaggctgaggcaggaaaatcgcttgaacccgggaggcagagtctgcagtgagttgagatcacgccactgcactccagcctgggtgacagagcgagactctatctcaaaaaaaattttttttaatgtattatttttgcataagtaatacattgacatgatacaaattctgtaattacaaaagggcaataattaaaatatcttccttccacccctttcctctgagtacctaactttgtccccaagaacaagcactatttcagttcctcatgtatcctgccagatataacctgttcatattgtaagatagatttaaaatgctctaaaaacaaaagtagtttagaataatatatatctatatattttttgagatgtagtctcacattgtcacccaggctggagtgcagtgatacaatctcggctcactgcagtctctgcctcccaggttcaaatgcttctcctgcctcagccttctgagtagctgggattacaggcgcccaccaccatgtccagctaatttttgtatttttagtagagatggggtttcaccatgttggccaggctggtcttgaactcctgaccttgtgatctgtccacctcggcctcccaaagtgctgggattacaggtgtgagccaccatgcctggctagaataataacttttaaaggttcttagcatgctctgaaatcaactgcattaggtttatttatagttttatagttattttaaataaaatgcatatttgtcatatttctctgtattttgctgttgagaaaggaggtattcactaattttgagtaacaaacactgctcacaaagtttggattttggcagttctgttcacgtgcttcagccaaaaaatcctcttctcaaagtaagattgatgaaagcaatttagaaagtatctgttctgtttttatggctcttgctctttggtgtggaactgtggtgtcacgccatgcatgggcctcagtttatgagtgtttgtgctctgctcagcatacaggatgcaggagttccttatggggctggctgcaggctcagcaaatctagcatgcttgggagggtcctcacagtaattaggaggcaattaatacttgcttctggcagtttcttattctccttcagattcctatctggtgtttccctgactttattcattcatcagtaaatatttactaaacatgtactatgtgcctggcactgttataggtgcagggctcagcagtgagcagacaaagctctgccctcgtgaagctttcattctaatgaaggacatagacagtaagcaagatagataagtaaaatatacagtacgttaatacgtggaggaacttcaaagcagggaaggggatagggaaatgtcagggttaatcgagtgttaacttatttttatttttaaaaaaattgttaagggctttccagcaaaacccagaaagcctgctagacaaattccaaaagagctgtagcactaagtgttgacatttttattttattttgttttgttttgttttttttgagacagttcttgctctatcagccaggctggagtgcactagtgtgatcttggctcactgcaacctctgcctcttgggttcaagtgattctcatgcctcagcctcctgtttagctgggattatagacatgcactgccatgcctgggtaattttttttttttcccccgagacggagtcttgctctgtcgcccaggctggagtgcagtggcgcgatctcagctcactgcaagctccgcttcccgagttcacgccattctcctgcctcagtctcccaagtagctgggactacaggcgcctgccaccacgtccagctaatttttttgtatttttaatagagacggggtttcaccgtgttagccaggatgatcttgatctcctgacctcgtcatccgccgaccttgtgatccgcccacctcggcctcccaaagtgctgggattacaggcatgagccactgtgcccggccacgcctgggtaatttttgtatttttagtagagatggggttttgccatgatgagcaggctggtctcgaactcccggcctcatgtgatctgcctgccttggcctcccaaagtgctaggattacaggcatgagccaccatacctggccagtgttgatattttaaatacggtgttcagggaaggtccactgagaagacagctttttttttttttttttttggggttggggggcaaggtcttgctctttaacccaggctggaatgcagtatcactatcgtagctcacttcagccttgaactcctgggctcaagtgatcctcccacctcaacctcacaatgtgttgggactataggtgtgagccatcacacctggccagatgatggcttttgagtaaagacctcaagcgagttaagagtctagtgtaagggtgtatgaagtagtggtattccagatggggggaacaggtccaaaatcttcctgtttcaggaatagcaaggatgtcattttagttgggtgaattgagtgagggggacatttgtagtaagaagtaaggtccaagaggtcaagggagtgccatatcagaccaatactacttgccttgtagatggaataaagatattggcatttatgtgagtgagatgggatgtcactggaggattagagcagaggagtagcatgatctgaatttcaatcttaagtgaactctggctgacaacagagtgaaggggaacaccggcaaaagcagaaaccagttaggaagccactgcagtgctcagataagcatggtgggttctgtcagggtaccggctgtcggctgtgggcagtgtgaggaatgactgactggattttgaatgcggaaccaactgcacttgttgaactctgctaagtataacaatttagcagtagcttgcgttatcaggtttgtattcagctgcaagtaacagaaaatcctgctgcaatagcttaaactggtaacaagcaagagcttatcagaagacaaaaataagtctggggaaattcaacaataagttaaggaacccaggctctttcttttttttttttttgaaacggagtttcgctcttgtcacccgggctggagtgcaatgatgtgatctcagctcactaaaacctctacctcctgggttcaagtgattcttctgcctcagcctcccaagtaactgggattacaggcgtataccaccatgcccagctaatttttgtgtttttagtagagatggggtttcaccatgttggccaggctggtctcgaacttctgacctcaggtgatccactcgcctcagcctgccaaagtgctgggattacaggtttgggccactgcacccggtcagaacccaggctctttcttatacttaccttgcaaacccttgttctcattttttccctttgtatttttattgttgaattgtaatagttctttatatattctggatactggattcttatcagatagatgatttgtaaaaactctcccttcctttggattgtctttttactttcttgatagtgtcttttgaagtgtaaaagtttttaattttgatgaagtcgagtttatctattttgtctttggttgctgtgcttcaagtgtcatatctaagaaatcattgtctaatccaaagtcaaaaaggtttactcctatgttttcttctaagaattttagagttttacatttaagtctgatccattttgagttaatttttatatatggttcaggtagaagtccaactttattcttttccatgtggttattcagttgtcccagcactgtttgttgaagagactattctttccccatggaattatcttagtacccttgttgaaaattaatcgtccttaattgtataaatttatttctagactgtcagttctacctgttggtctttatgtcgatcctgtgccagtaccatacagtcttgattactgaagtttgtgtcacagtttaaattcatgaaatgtgagttctccaactttgttccttttcaagattgatttggccatgctgggtcccttgcatttccgtacgaattgtaggatcagcttgtcagtttcaacaaagaagccaagtaggattctgagagggattgtgttgaatctgtagatcaacttggggagtattcgcatcttaacaatattgtcttccacctatgaacatgggcaaactttgtgtaaatggtcagattgtaagtatttcgggctgtgtgggcacagtgtctctgtcacagctacgcggctctgccattgtagcatgaaagtagccataagcaatatgtatgagtgtctgtgttccaatagaattttattaatgacaaggaagtttgaatttcatataattttcacctgtcatgagatagtatttgattattttggtcaaccatttaaaaatgtaaaaacatttcttagcttgtgaactagccaaaaatatgcaggttatagttttcccactcctaggttaaaatatgataggaccacatttggaaagcatttctttttttttttttttttttttttttgagacggagtttcactcttgttgcccaggctggagtgcagtggcgcgatctcggctcactgcaacctctgcctcccaggttcaagacattctcctgcacggcctccctagtagctgggattacaggcatgcgccaccacacccagctaattttgtatttttagtagagacggggtttctccatgttggtcaggctggtcttgaactcctgacctcaggtgatccacccgcctcagcctcccaaagtgctgggattacagggtgtgagccaccacaccctgctggaaagcatttcttttttggctgtttttgttttttttttaaactagttttgaaaattataaaagttacacatatacattataaaaatatcttcaagcagcacagatgaaaaacaaagcccttcttgcaagtctgtcatctttgtctaacttcctaagaacaaaagtgtttcttgtgtcttcttcccagattttaatatgcatatacaagcatttaaatgtgtcattttttgtttgcttgactgagatcacattacatatgtatttttttacttaacaatgtgtcatagatattgttccatagcagtacctgtaattcttattaattgctatgtaatattttagaatttctttttaaaagaggacttttggagatgtaaaggcaaaggtctcacatttttgtggctgtagaatgtgctggtgacatattctctctaccttgagaagtccccatccccatcacctccatttcctgtaaataagtcaaccacttgataaactacctttgaatggatccacactcaaaacatttagtcttattcagacaacaaggaggaaaaataaaataccttataaagcactgtttaatattgtattaaattggatcaatttgggggctagaatgtatgttagagacatgatatgtccataggtccttgctatcacagtgaggtctcagggacagtcgtttggtatcatttgggatctcataagcagactctctctgcttgacctgacaaatcagagtctgtgttttaacaggttcagtgagtgacttacatgcacattggagtttgggaagctccactgtaggtgcttagaccttacctttgttgttgctaataacaatgcaagcatttgggaggaagacctgtgttgctcatatgtgtccaggtgtagctgaggtggccttgcttatctgctgtagggccgttgagcatttctgtagctgtgatgagtgagctgaggtgagcctgcggagagctcccagccattggtagtgggactcgcttagatgaactggaaggaccctttcatctgagcagccactatggagaaaaacaaccgaatgaggggagagacaatgtgcaattttatttagggcacaaaggagagctgtggttagaaggtgacatttgagtggaaagggggcaagccatgtgtatagcgggagaagagaggtccaggcagagttaacagaaggcagaaatgctttccatgtttgagaaccagtaaggaggccagtggctgaagtaaggtgaagggcagaaataaggatgaggctgcgagagatgagaggttagagacgagcgtcttgtgcaccaagataagcttgtgtggtcaaaacaagtagtttaatttatgtttttaaaagatcattttggctgggcacaatggttcatgcctgtaataccagtagtttgagacggtgtggtgggaggattgcctgaggccagacgaccagcatagccaacatagcagcacctataaggtctctacaaaaaactttaaaaaattagctgggcatagtggtgtgtgcctgtagtcccagctactcaggaggctgaggaggctggaggattgcttgagtccaggagtttgaggctgcagtgagctatgattatgccactacactacaacctgggcaagagagtgagaccctgtctctaaatatacacacacacacacacacacacacacacacacacacacacacacacacacacacacatatatatgtatatatatgcatttagatgaaaagatcactttgacaataccacatgctggtgaggatttagaaaaactaggtcacttattgctggtgggaatataatatagtacggccactctggaaaacagtttggcagtttgtcataaaactgaacataccgttagtatacagcccagcagcaactacaatcctgggcattaatcctagagaaatgaaaccttaatgttcacataaaaacctatactcaagtatgcatagcagctttacccataatatctaagaactggaatcagctcagatgtccttcaacaggtgaatggttaaactactcagtaataaaaaggaatgagctactgatagcatgcaacagtttaggtgaagttatgctaatgaaaaaagccaatcccaaaaggttatacatactgtatgattctatgtttttttgcaatggcacagttttagggatggagaatagattagtggttgcctggggttagagatggggtagtagagtaggttagtggtggcagaggagagaaaagagagggaggtgaatgtggttataaaaggacaacacaggggaatacttgtaatggaaatgctttgtctttttttttttttttttttttttggcgacagagtcttgctctgttgcccaggctggagtgcagtggcatgatcttttctcactgcaacctctgcctcctgggttcaagtgatacttgtgtctcagtctcccatgttcagagtgaaacaaaccagaggtaatgttcatccaaataatccaacacacatgacattaaaacatcaagatcaggtcggacgtggtggctcatgcctgtaatcccagcacttttgggaggccaaggtgggcagatcacttgaggtcaggagttcgagaccagccgggccaacatgatgaaaccccatcttgactaaaaatacaaaaattagccgggcatggtggtgtgcacctgtagtcccagctacttgggaggctgaggcaagagaactgcttgaacccgaggggcagaggttgcagtgagctgagagtgcgccattgcacttcagcctgtgtgacagagtaagactccatctccaaaaaaaaaaaaccaagatcaattaaaatacagcattactgggccgggtgtggtggctcacacctgtaatcccagcactttgggaggccgagatgggcagatcacgaggtcaggagatccagaccatcccggctaacacggtgaaaccccgtctctactaaaaaatacaaaaaattagccgggtatagtggtgggtgcctgtagtcccagctacttgggaggctgaagcaggagaatggtgtgaacccgggaggcagagctggcagtgagctgagatcgcgccactgcactccagcctgggcgacagagcaagactccgtctcgggggaaaaaaaaaaataaataaatagaatgctgtagtgtccttgagtttacatgcccctccttacgcttgtgtgcccgtgcagattgcttgattacacaattagaggaggctggcggaggattgttttaatttttttttttttgagacagtctggctctgttccccaggctagagtgcaatggcgcaatcttggtgcactgcaacctctgcctcctgggttcaagcagttcttctgccgcagcctcccgagtagctgggattataggcgcccgccaccacgcccaactattttttgtatttttagtagagcagcgtttcaccatgctggccaggctggtctcgaactcctgacctcagatgatctgctgccccagcctcccaaagtgctgggattacaggcgtgagccacacctggccgtttgttttaattttgaaggtgaagtgaaagtgactacatttaccaaaagtgattgaaaagccaggactgttcttaccctgtttttccagttcttgctcagagcaaggtggtttctttttcacttaatcaccatacttacttttcatgtagaacaagtcagtttgagttatcagttcatcatcttaactaaattccatgggggaaggaattagttttagtttcttaaacttccaggtttgcttattggacaaaatgagatagcaaggcagtgtttttaagttagattttttatttctttggtaatacaattttctcagaaacttagtagtcttttagtttagttgtttttagttggtcctatgttttggatcacccctctctactttattttgatagtgccaactgtgaagacatctgaagccataggtttggatgggaaggaggcatctttagcctgatcatcttcgccaggctgtttatctccttttgcttggctgagaagtcttaataggaggcttattcccagctatttggggacatagaagcagttagccattgcttatattttactgaggtctgtgtggtatgttgattgtagtcagttaacgattttgagaactgaaggcagcctggtatatatagagtaggtattagactgtgtttcttctaattgaatttcccatctcttgtaatctatgccatcatcttctgtactgctgagaaagaaagaaagtttctaatcaaactataccactggttgtaagatgcagtttggctttagtgatgttaacacatgattcaaacgtgaaattgattgagtattggtgaaatacagaggagatttaaagccagaagacctgggtttaaatgctggctgtatgacttcatatctgtgtgatcttgggcatgtcatggttggcacttcaatttcttctctctataatgggggaagtgaggccagtcatggtggctcatacctataatcccagtgctttgggaggccaagatgggaagatcgcttgaggccaggagtttgagcaattgggcaacatcgtgaggccccgtctctacaaaatattttgaaaaaattagccaggcccagtggtgcgtgcctgtggtccgcgccactcaggaggctgagacgggaggatcctttcagcctaggagtttaaggctaaagtgagccatgattgtgctatcgtactccagcctgggcagcagagcaagatcctgactctaaaaaaaagtaaaataaagtaaaatgggggaaatgaactgctttagtaacatcatctgttttttctgtgagcagcgtagcttgacagccattggtgaactcgtgccctgtgcttccctgtccagatccccattctgcccgcaacatggagtataacggtttattcatagtagtcgagaaacactcactgaatgaatgaatgaggtgtagaactaagtggagtgggtaattcaacacatattaatttccttcttttttttatttttagaaagaaagaactttcagctaccaagaaagaccgtgtgaatcattgtctgacaatatgtgaaaacatagtggcacagtctgtcaggtaattgcactttgaactgtctagagaaaataagaactttgtatattttcagtcttaatgggctagaatattctttgtgtcccagctattttaaatggattcagaaatccatttaagatgaagaaggacccttttcccatatttctggctatatacaaggatatccagacactgaaatgaataatgttccctttttgtaatcttttatgcaaaaattaaaaccattatggtaattgaacaacatgtttatgtttagttaacacccttagcaactatagttattttaaaaccatctatggtttgatatttttgcatttgttgcaatagtaggaacagcacaagacagttcagtttgtctctcttatttgctttttcttggcagtttgctgtcctattgtacctctgctcctagcagtggctggagcccactcctctgtgcttcgggattagtggggatcgtggggcattgactgtaggtcagctttccttgcttgatctttctcactgggatgaactagcagcaccttcttttgtagctgctttgcttttgactatctttctgaccgttgttcctagtagctgtagatggtaaatatatttaggcctgtttccaatggctcagtaggagacatattcacctatgatatctgaattctgttacccacatgggcatgcgtgaaatagttgccttgccttactttcccttggaataaataattcatgttattctcctggtagaagctagaaaaagcctttatagtcagtcagaaaaaaatttttagacaaataatcttgattttagtactgacaaaaacgtgtggtgattctttttttaatttttttttgagacggagtttcactcttgttgcccaggctggagtgcaatggcgtgatctcggctcactgcaacctctgcctcctgggttcaagtgattctcctgcctcagcctcccaagtagctggagttacaggcatgtgctactgtgcccagctaattttgtatttttagtagagatgttggtcaggctgatctcgaactcccaaccttaggtgatctgcccgcctcagcctcccaaagtgctgggattacaggcgtgagccagggcgcccggtgattcatttgttttttcaaaaaatttcctcttggccattgcttttcacttttgttttttttttttttttgagacggagtcacgatctgtcacccaggctggagtgcagtggcatgatcttggcttactgcaagctctgcctcccaggttcacgccattctcctgcttcagcctggcgagtagctgggactacaggtgctcgccaccacacccggctaattttttgtatttttagtagagatggggtttcaccgtggtcttgatctcctgacctcatgacccgctcaactcagcctcccaaagtgctgggattacaggcgtgagccaccgcgcccggccctctcttgtctttttattgtggtaaaatgcacataaaattgactgtcttaaccatttttaggggtacagttcagtatatatattcgtaatgttgtacagccatcactgccatctacttcataagtttttcttctgtcaaaactgaacatctgtcttcattaaactccctatcatccattctttcctgtagtccctttctactttctgtctgtatgagtgtaactgctctggagacctcatgtaagtggattcctacaggatttgtgttttttttttggtgatctgcttatttttaatgcctctgtgcatttgtattatatactttcaaagtgatttcacaaaaccgtttcattttaggttaactcatttctgttgtttgtgaaatactgtgtatgattctgttctgtttctgtctaatttgtggaaatgttgtgggaagaaaatgaaataacaaatgagcatatgtcctgaaaataaaaatataaaaattctaagttagcatgctattgtagaatacaacgctatgataaaagtaggaaaaaaaaaggtttgaattctatctctgctacctgtgtaagctgggtgactttagataagctgtaacgtgtttgagccttactggctcatttttgaaatgtaatccctagttacacagttcttgtgggatcagatggtacatgtgaaacactgtgaaaaagcaactgcatagatatgttcattagccacctgagcgggaagcgtatcccattgcgatgcccatcatccaaagctatatgttatctttacttttttttttttgagacagagtcttgctctgttgcccaggctagagtgcagtggtgcaatctcagctcactgcaagctccacctcccgggttcacgctattctcctgccccagcctcccaagtagctgggactacaggcacccgccaccatgcctggctaaatttttgtatttttagtagagatggggtttcaccgtgttagccaggatggtcttgatctcctgacctcgtgatccgcccgcctcggcctcccaaagtgctgggattacaggcgtgagccactgcccctggccatctttactttttttgtgaaatgactttaaatacttggcaaacatttggtcattgttcatctgatctccaccatccaggtctcagagaacataatttctctctgaaagcttattgacccaggaaataagatctctttcaatctgagtgcgtcaggctttattcttgtcattttgtcttttgataattttcaaatggaattcatggaatgttggcttatattcatatattagtaaagtatgttgagacatcttaagattgatttgtggttctatatgccatattaaatcaaaataatagctgttaatggttttcacattagtctgtctcttgtttttatggagtaatgctgagagttcattatgcttgttctacagaagagcatgttaaaaggagtttttggagtcagagaggttattcttggtttcataggatacactctatactttttagggatttcagagtatatagctgaaggtgatattttatgtaaatatgttttatggaaacttattgctcatcgctgtttcctgttaactctcctaaaatataattaaacttttggaacttttttatagcttttgtgctagactaatttttgtctctaatgaggttatataaatggcagcttctgacgttttcaatgtaggaagtcatttaaaacttcatgtatattgtgaaaatgtagtctgctttaagctctctaaagtggtctaagttactggttcctaagtatggatgagcatcaaaatcatctggaaaatttgttaaaaatacagtaatgaaggcacctcactgtcctttttcccaaacatacttctgcattctgtttgagtaggtagggactacacatttttcacaagtatcctcttgggaatacccaggaatgcttacttgagcaacctcttactaatatgtaccttgataaggtggctaggtaaacataaatatacaaaaatccatagatctcccatatattagcataaatcagctagaaaatataacgtttaaagatctagttcacagtagcaccaatatatcgaactctaaggaatcgataaatatgcaaaaactttataaaaacttctgttaatgtttctgaaagatataggtgaccactttctagataggaagattttatattactaagttgaattttctctaaattaacacagaaatttaaaataatcttgatcaaaattctagtagaggtatttttgaacttgttcactgcaagaataaatacataattgcaaagaatatctcaaaatcatcaccaggcctggtgtggtggcccatgcctgtaatcccagcactttgggaggctgaggcaggcagatcacctgaggtcaagagtttgagaccagctggaccagtgcggtgaaacactgcctctactaaaaatacaaaaattagctgggtgtggtggtgcatgcctgtagtcccagctacttgggaggctgaggcaggagaattgcttgaacccaggaggtacaggttgcggtgagcctagatcgcaccactgcattccagcctgggcgacaagagcaaaattctgtctcaagaaaaaagagaaaaaagaaaaagaaatcaacactaatatggtgagacttaatgtatgtgacattaaaatagtgattggatgttaaaacaggtatagaacagaaagaagagtgtatgtgtgtatctgtatgaatttatgatgggtgtaacatatatgtattagggaaatgagggaaatgatacatttctctgactttgggagaacattatatctctacctcatattgcaaacaaacataaagttcagattaattacctaaatgtgaaaaaatgaaataatttctttaaaaaatgtaatcttagtttgaggaaggttaacattataaaggaaaaaactgttttgagtggaatatagttcaatatgtcaaaatccaccttcaacaaaattgaaagtaaattgaacttggggaaagtattgacagcatatagatcaaaggttactagcctgtgtaaagagcagttataaatatcgttaagaaaaacactgtcgacctgtcggcaccttgttctccgactcccagcctccagaactgtgacgagtaagtgcttattgtttaaaccacccagtctgtatgtggtattttgttatagaaactcaagctgattaggacactagtaatcagtagactgaaactgaaacaaaaataagaaccttttttacctgtcaaattggcaaacattaagaatattcagatttttgtcagaggtgatacaaccttctaagaaggcaatttgggaaaatataaagctttagattattatatgtctgacctagcagttttacctctagggtgcttacccctaggaaagtgtgtaatgatattggtgcagtgcccttcatcccattagaaaattaaaaataaccttaatggcctaccactaaaaggggattgaaaatttaagatatatttatttatgtgtttattgagatggagtcttgcactgtccgcctgggccagagtgcaatggtgcgatctcggctcactgcaacctctgcttcccgggttcatgtgattctcctgcctcagcctcctgagtagctgggattacaggctcacaccaccgcacccggctaattttttgtatttttagtagagatggggtttcactgtgttggccagactggtctcgaactcctgacctcatgatccgcgcccctcggcctcccagtgttgggattacaggtgtgagccactgcgcctggccagatacatttatacaagagaatgttagttaacattcatagatatttatattttgtttactttttattaaaaaaattttttttagagacaggatcttactctgtcacccaggcaggatgcagttgcacaatcatagcccactgcagcctgaactcctgggcttaagtgatccttctgcctcagccttttgagtacctgggggactttaggcagtgctactatacctggctaatttttaaatgttttatagatgagatcttgctgtattgcccaggctggtctagaattcctgggcccaagtgatcctcccaccttggcctcccaaagcgctgagattacaggcatgagccaccacttctgaccaatagatatttatatttgtgactggaaaatatattaacaatgtgttaaaaaattcagttaaaaaataatgaaagatttttgcttctggctaagatagaataacaaggacagcatttatcttcttgccttgaaatagttgaaaacggaagaaatatatgtaacagtggttttcaagttattgggcatcaggcaaagaagaatagttatcccaggaaaatgaatgtggagagccctacaatttccttacattactgcctggtcatggcaagaggaaaaactgagaggagactgaggctgagccagtggtttgctgggttgaggaggcagagctgggagtgcagagatgcaaggtggtgagagcccatatggaagaataccagggaagagagctgcagagggagctccggagacctgcaccctgccctctcagtaccctgtcatgtgtgtagctgagtactgacgagcacttgcttgtgcggaaatgacccagggctggaggtagagccacctgaaaggattagaaggaacagttgctgaaagtcacacagggccaggaagaatttctaatcacaccagttggagtggaaaacctcagctctcatagagcaggtagggtactcagaagggtttgcccacctagccccagactaagtttcgttactctgaccctacctaatattaaaaagagattaattaaattgttcgcaacaaaaataatatatttcagtgtttgtaacacgtagaagtgaattgtatgacaatagcataaaggctggaagagcagaaattgacatgtatttgcgctgggcagaataatgctcccctctttccccaaaagatatcaagtcctaatccctggagcctgtaaatattactttatatggaaaattgttttatgatgtgattaaattcaggatcttgagatgagggggctatcttggatgatctgggtaggcactaaatgcaatcacatatatataaaaaggaggcagagggagattttacacacagagagaaggccctgtgaagatggaacagaaagatttgaaggtgctggccttgaaaattggagtgatgaagctataagccaaggaatgcagcagccaccaaagctggaagaggcacggagcagttctcatttagagcctactccagagggaatgtggtgctgccaattccttttttttttttttttttaagatatcatttacccctttaagttggttttttttttttttttttttttttagtatttattgatcattcttgggtgtttcttggagagggggatttggcagggtcataggacaatagtggagggaaggtcagcagataaacatgtaaacaaaggtctctggttttcctaggcagagggccctgccacgttctgcagtgtttgtgtccctgggtacttgagattagggagtggtgatgactcttaacgagtatgctgccttcaagcatctgtttaacaaagcacatcttgcaccgcccttaatccatttaacccttagtggacacagcacatgtttcagagagcacggggttgggggtaaggttatagattaacagcatcccaaggcagaagaatttttcttagtacagaacaaaatggagtgtcctatgtctacttctttctacgcagacacagtaacaatctgatctctctttcttttcccacatttcctccttttctattcgacaaaactgccaccgtcatcatggactgttctcaatgagctattgggtacacctcccagatggggtggcggccgggcagaggggctcctcacttcccagatggggcggccgggcagaggcgccccccaacctcccagacggggcggcggctgggcgggggctgccccccacctcccggacggggcgggtggccgggcgggggctgcccaccacctcccggacggggcggctggccgggcgggggctgccccccacctcccggacggggcgggtggccgggcgggggctgccccccacctcccggacggggcggctggccgggcgggggctgccccccacctcccggacggagcggctgccgggcggaggggctcctcacttcccggacggggcggctgctgggcggaggggctcctcacttctcagacggggcggctggtcagagacgctcctcacctcccagacggggtggcagtggggcagagacattcttaagttcccagacggagtcacggccgggcagaggtgctcttcacatctcagacggggcggcggggcagaggtgctccccacttcccagacgatgggcggccgggcagagatgctcctcacttcctagatgggatgacagccgggaagaggcgctcctcacttcccagactgggcagccaggcagaggggctcctcacatcccagacgatgggcggccaggcagaaacgctcctcacttcctagacggggtggcggctgggcagaggccgcaatcttggcactttgggaggccaaggcaggcggctgggaggtgaaggttgtagtgacccgagatcacgccactgcactccagcctgggcaacactgagcactgagtgagcgagactccgtctgcaatcccggcacctcgggaggccgaggctggcagatcacttgcagtcaggagctggagaccagcccggccaacacggcgaaaccccgtctccaccaaaaaacacgaaaaccagtcagacatggcggtgcgtgcctgcaatcccaggcacttggcaggctgaggcaggagaatcaggtagggaggttgcagtgagtagagatggtggcagtacagtccagccttggctcggcatcagagggagactgtgcgagggcgagggcgagggcgagggaattccttaatttcagtttagtgatactaattttggactctggcctctaaaactgtgaaagaaaaaattttttgtttgtttgtttcttttaagccacatagtttgtggtaatttgttacagcagctgcaggaaactaatttatgctgcatgtgaaatggtgtaataaggtagattgtgatgaagatacatagtataaacaattaagcaacaactaaaagcacaacaaggaattatagctaatgaaccaaaaaaggagattagaataataaaaatggtgaatcccaaagaagccagaaataggggaagaggcaaataaaggaaagaaagagcttgatggtagatttcaacctaactatgtcaaaaaggacattacatgtaaaaggcagcgatttttcagattgaatggaaaagtaagactcggtatatgctgctgcctgcaagaaacacattctaaatataaaggcaaaaataacctacaggtaacagaacggaaagaagttcactgtgcttacaagaattagatgcaagctagactggttctgttaatatcagacaaagtggatttcaaagcaaaggctcttgcccaggatgagatggtcatttcataatgatgaaggggattcgttcatcagcctggcatagcaagctgaaatgtttatgcaccggactacagagctaaaatacatgaagcaaagcctgacagaactacaagtagaaacagacaaatccacagtgatagagatttcagtagccgctctcaatgatttgtagaacacgtagccataatatctggatctagaacacttgaccaacactgtcccctgtgcaacctcattggcatttacaggacactccacccagcaccagcagaagagacactctctcaagtgctcacagaatgtttgccaagatagagcagatgctgggccataaaacaagtctctaaattaaaagcattcaaattattcagagtatgttttctgacctcagtatcattaagttggaatatattataggaagataacctggaaaagcctcagatatgtggaaaaacccatttccacatggcccatgggtcagaagtgaagtcaaaagggaaatttgaaagtcttttggattgactgatataaaaacaatagatttctaaacttgtggggtgctgttacagcatagtaaatggaaatttctagcattaaatgcctgttttaggaaagaaagatttcaaatcaatgacctcagcttctacctttggaaacttgaaaatgacaagcaaatggaatccagagttaccagaagggccaggtacggtggcttatgcctgcagttctgccactttgggaggccgaggcaggtggattgtttgagactggcagttgaagaccagcctgggcagcctagggagaccccatatctacaaaaaacaaaaaaattagccaggtgtggtggcatgtgcctgtagtcccagctaaccaggagtctaaggtgggaggattgcttgagtctgggaggttgaggctgcagtgaactgtgattgtgccactgtgttccatcctgggcaacagaatgagaccctgtctcaaaaacaaaaacagttactagaagaatggacatcataaagataggagcagaagtcagtaaaatagaaaacaaaaatacataggaaatcaataaaaccaaaagctggttcatcaagaacatcaataaattggtaaagctgataggaaaaacagtgaagtcacaaattagcaatatcaggaatgagggagatgacagtagtatagattatatagatattaaaaggactgtatgaggcaggtgtggtggttcacgcctgtaatcccagcaccttgggaggccgaggtggacagatcacctgaggtcaggagtttgggaccagcctggccaacatggtgaaactctgtctctactaaaaatacaaaaattagttggtcgtggtgctgtgtgcctgtaatcccagctacttgggaggctgaggcaggagaattgcttgaacctgggaggcggaggttgcagtgagctgagattgtgccgttgcactccagcctgggtgacagagcaagactccatctcaaaacaaataaataaataaaaaggactatatggtaatattatgaacaactttatgccaataaatttgacaacttatagatgaaatggatgagttccttgaaagacacagaaactattaaagctctctcaagaagatatagataagctgattagccctatatctattttattgaatttaaatgtaaaaatcaatatttagttactggaaaacttttaagtgtggttggaaatggtatacgaactttttcaactgaattttatgaagtctaatcacaggtaaaggttttctgatgaaaatttagtgtctgaattgagatatactgtaaaaaatgttatatatcttaattatttcttcacattaattacatgttgaaataatactttgggtgtattgggttaaattaaatattatgaaaatcttgcctgttttctttttacttttgatgcgtcagctaggaaatataaaagtgtagctcacattctgtttctgttgacagtactgctttggagcacagtgtttgaatgatctatcatttcaaagacctttcctcagttcgttattcatggctgtctgtattccacatagataaggtctgaaatactgctaagtggcatgttttgttttatgcttttataagtttgttgatcattactgatgtggacttttggtgcctcttaggctcattgctatcttccaaccattgtttgcaatttttacctagagataaagagaaagagacatttggtttcagagtagttagattgggatcatgaaagagcaacctcattttgatgcttcaaaaatagcacatcccccgtattactgggatttgctattcttgggattacttcaagaacatccttgtgttactggtttggatgcttctgaatgctgtgaagtcagtttcatgtacatggctcatcagtttagctctctcttggctttgtttagacagttggagcatgatggcctaaacagcttctttcaattaaacattttaaaatagtttacaaatagtaaacaaactccagtttttgtgactctttgtctcgcacaacaaaaacacaatctgaccatgatcatctggcatcttagggtgaaatatggttatactttggcccataccgaaagcaagattaaaaaggggcaggagagatagactgctgaactgattttcaaggttccaagaatattgtaggttaagagtaaaagtaaacttttggtagaaagcagtgggttgtctaggattgaagtatctgaagtttttaaacgaaaatttaaaaagaaaaatgagaattgccttacaagtacaatctcttcttttttaaaaaataaactttattttgaaatagttttagatttatagaaaaaaattagatagggtaggaagttttcatataccctacatccagttaccccagttattatcatcctaatttagtgtgagacattttcatgtttaatgaatcaatattgatatgctattaacttaagtccagactttattcagattttcttaatttctatgtaatgtcctttttctgttccagaattccatgcaggacaccggatacctcattacatttcattgtcatgtcaccttaggctcctcttgacagtttctcttctttttttgcttagaaattctccagaatttcagaaacttctgggcatcgctatggaactttttctgctgtgcagtgatgacgcagagtcagatgtcaggatggtggctgacgaatgcctcaacaaagttatcaaagtaagaaccgtgtggatgatgttctcctcagagctatcattgttgtaggctgagagaagaagcgatcattgagtgttcttctgttttgagtccctgaggatgtctgcacttttttcctttctgatgtatggtttggaggtgctctgttgtatggtttggaggtgctctgttgtatggtttggaggtgctctattgtatggtttggaggtgctctgttgtatggtttggaggtgctcttgtatggtttggaggtgctcttgtatggtttggaggtgctctgttgtatggtttggaggtggtcttgtatggtttgcaggtgctctattgcatggtttgcaggtgctctattgtatggtttggaagtgctcttgtatggtttggaggtgctcttgtatggtttggagatgctctattgtatggtttgcaggtgctctattgtatggtttggaagtgctcttgtatggtttggaggtgctcttgtatggtttggaggtgctctgttgtatggtttggaggtgctctgttgtatggtttggaggtgctcttgtatggtttggaggtgctctattgtatggtttggagatgctctggtatctgcctgcattgcttgccacacctgcccggtcagaaggcgctatgttgacaattgtgcctgcacggtgcctaggtcaatgaagggaaccgatggtagccactggatgctcctgggaaaatgtcactacaggcaccagagaagccagagctatgcccaaatttctatgagtctcagttttcttaaccataaaatgggatcaatgtttttgtggcatgtgtatgagtgtgtgtctgtgtatgtgtgaggattaaattgtgtatgtgtgaggactaattgccactactggatcctcaaagtggtaagaagtgttcttattaataatgacatccttacactcttacccagcaagattgatgggtgtggcactgcttctctttttccatcacatggtttccatggtatccttttgcccagggaatctttgctttgtggctagcactttgttgtttggctaatcacgctttctgtggtcaggacgctggcttctctggagccatgggattctagctccctgtcttgtccctagagtggtcactgtcttctctctccgcttgcaattcctgctttgctcgcatctcacttatgcagtgacgtatatcagtttcaccttgttctccgtgcctgctgatcattggcaccacttgcatggtgccatttagggcctgcttccagttaagcttgcttctccacaggcctaaatatccttgcttgcttcttttattctcactggcaggaccagggcggtctgtctttgcatgagacagggtctcgctcagtcacccaggctggagtgcagtggctgatcacggctcattgcagccttgagctaccgggctcaagctatcctcctggcttggccccttgagtagctgggactacaggcgtgcaccaccatgcccagctaatttttaaaattatttgtagagatgggatctcgccaggttgcccaggctggtcttgaacgcctgggctcaagtgatcctccctccttggtttcccaaagtgctgggatcacaggtgtgagccactgtgcctggcccttgatgtttcagttcttgatatttgatcctcagagtcagaaaatctaaaaagagggctatcccaggttgccttggttcatggcaaatgggacgttaagagggcagagagaatatgaacagaaactgttctaatattggtcatttaatgtgtaagtattgttcttttttaaacctccttcattttttttccaggaattgctggacacagtggcttggtgtgtgtctgaggactgtaggccatggccctaggttgtggttttaggtctcaggtgctcttcctggctgtctccttgcttctttcccatgtcctcttctttgtttccagccatttctcccttatgcttaagtttggtgcagcagggtttggctgctctcagattcctgcttcctcagatgctgtagttgtcaggcccagcgggctggcagcgggatcaggatctggctaggtttgctctcactgtggcagagtagggggaggcgtgggagagcacgtgtgaccccaggccagctgtagggagcataggcatggtcacgtagccttcaggtcctagactttgtcttctcatgagtatggctgtgtgtgtatggtgaaaactaggttctacttagcccaagaaaatgggcacattttgcatgtggtttctgtagagaaatgcactgggtatctgacatagcctggcagcatgcctccctcaggtaggttagtctcaggcggtgaagcacgtgtgtccagcaagaacttcatatgtggcataaagtctccgttctgtgaggtgctggcaaatcaccaccaccgtcaagaggctgaagtgatttttgtctagggaggcaggaaaggcttcctggagtcagcagccagtaggtgaaagagtagattggagaccttcttaatcatcaccgcctcttgtctcaaggggtgccaggaagctgtggaggctgaacccatcttatgctgccagagagtgggacaccatgagggtcaggtcaaggggttgtaccttgtttggtagagaattaggggctcttgaagactttggatgtggtcaggggagtgtatcatttaggaagagtgacccggtgaggacgtggggtagaggaggacaggtgggagggagtccaggtgggagtgagtagacccagcaggagtgcagggcctcgagccaggatggtggcagggctgtgaggagaggcagccacctgtgtgtctgcggaagcaggggcaagagggaagaggccagcagcgtgctgccatcacccagcgactggcgtagattgtgagagaccattccctgctcttaggaggggctgagttttagttttctcttgttatacaataagcttggtatttgtttacaaaacatttgtaaagctaaatcaaggtttgataaggcttctagttttatttaagaagtaatgttgaaataaatgtttgtccaattcgctttgctcatttaaggactttcagtacaaactgcaacaacaggattaggatttaaacgtttctgagatgtttttactcctcagaatttcccagaatgtgatctggttttgattttcaagcttgctgacccaataggttaacccacaagttttacgaagaccatctcagtccacttacatcaactgcccatgccacggttaaagagatcatcgactgatgtttggcacagcttcctccctcttgggtgggcaagcatttggaagagaaggctcctatgggtgagagtggggcaccaaagtcttccctgtcccatcccctagcttgagaagcccttctctaatgtggactttgtgccgttagcatcgttactagcttgaagttgaccatctggacgtactttctggtttagcctcacaagtgagcaaggagggttgagagatgtgctgtgaggaatgtggggccccagctggcagcaggctctgggtcaggggggcagggaccacgggcatacctgacagtgaggaggggccacacctgcagaaaaggatgcaggactccgccttgggaagtgttctaggccagagcgagggtctgtggtttataagtacacccacagtgctcgggaccctgcagatgtccagggtgccgtctgagcccgtatcatccaacagaatgttctgctagtgaagattaaagatttactccaggggctttaggatttattatatatatataaatcctatatatataattttttttttttttttttttgagatggagtttcgctcttgttgcccaggctggagtgcaatggcgtgatcttggctcactgcaacctccgcctcccgggttcaaactattctcctgcctcagcctctcgagtagctgggattacaggcgcccaccaccacacccggctaatttttgtattttttagtagagacggagtttctccatgttggtcaggctggtcttgaactcctgacctcaggtgatctgcccgccttggcctcccaaagtgctgggattacaggcatgagccaccccacctggccaggatttattgtatttgaaccatctaccattttaattttgatgttatgtagtatttgatgataatgaaagttaaattgtttttctttccatttttctgtttaagtgaatgacctgtatctagtttattcagtaacttcctgcatatatttgtttctttcattcttaatgaatatattcttaatttagttgctattatgttttgctttgccccaaaattgaaatcttagtttccttttagctcgttttagaactagtgatgggatgtgtcttccataaatctcttgtgatttgttgtaggctttgatggattctaatcttccaaggttacagctcgagctctataaggaaattaaaaaggtgggccttgcttttcttttttaaaaatgttttaaattttaaatttttataggtacacgtattttgtaggtacatgtaaatgtatatatttatggggtacatgagatattttgatacaggtatacaatacataataatcacaccatggaaagttggatatccatgccctcaagcatttatcctttgtgttacaaacaatccagttacatgctttacttattttattttatttttgagacagagtcttgctttcacccatgctagagtacagtggcatgaccttggctcactgcaacctccgcctcccgggttcaaccgaactttgggctggtctcaaactcctgacctcaggtgatccgcccgcctcggcctcccaaagtgttgggattacaggcgtgagccactgtgccgggcctgattgtacattttaaaataactaaaacagtcagggcacagtggctcatgcctgtaatcccagcattttgggaggctgaggcaggtgatcacctgagatcaggagttcgagaccagcctggccaacatggagaaaccctgtctctactaaaaatacaaaaattagccaagtgtggtggcgggcgcctgtaatcctggctactcgggaggctgaggtaggggaatcgcttgaacctgggggtggaggttgcagtgagccgagatcacgccactgcattccagcctgagcgacagagtgagactttgtctcaaaaaataaaaatgaaataaaattgggccgggtgtggtggctcacaccttagtcccagcactttgggaacctgaggcaggtggatgcttgagaccaggagtttgagaccagcatgggcaacatggcaaaacgctgtctgtacagaaattagctgggtgtggtggtgcacaactatagtctcagctacttgggagattgaggtgggaggattaattgagcctggaaggttgaatctataggtagctgagattgtgccactgcccttcagcctgggcgaccaagtgagaccctgtctcaaaagaaaaacaaaaaaacaaaaaacaaaccactattatcgactatatattattgtctatgatccctctgctgtgctgtcgaataccaggtcttgggcccttatttccatcactgagcaaacttcactctgttaagcagcaggtgtgggatttcatcgttattcagtaattcacaatgttagaaggaaatgctgtttggtagacgattgctttacttttcttcaaaaggttactctttattagatgagatgagaattaaaaatggtaacttactttatatctttataattgaagcccactagaccttaaagtagttaccagatgttttatgcatttaaatggccttttctctaaaattagaaagtaacaaggaaagaaaatgcttcgtttctatgcaaccctcttggtgactagtatgtgactcttaatgcaaccctcattgcaccccctcagaatggtgcccctcggagtttgcgtgctgccctgtggaggtttgctgagctggctcacctggttcggcctcagaaatgcaggtaagttgtacactctggatgttggtttttgtcgggggccagctgctactgatcctttatgtctcagctcagatgtcatttcaaaagtctgctctgccctctccaaattgcagtcgaccttgccctgtttatgtttccctcatagcactaatccatgtcagaaattgtcacgtacagtctatctgtgtgcttgtttattttctatcccacccttccgcaagagacttatgggatgtgtgccccaggacagcaggggtcttactgtcttatgctctgttgcagcccagcagcgataacagtgtctgcacatagtacttgcttaaaagatacttgccaaattgttgaaggttgaggtaccaatttcattattgctgactataggagttatagcaaaatatccatttgtctgttacatgagttaaaaatatggttgttgcactgtgaatagtttggtttagtcaaaacagttgtatcttaacggattgagaaacaaaagcaggaccacttttcatcagctccctccttctccttaaccagcaatacatgctgatgctgatatcccatagaccctcagctccatcctgagtcactgggaatgtggtctaaaccctcactattaatatgaactgagtttcaataagaatcttatatgggtcgggcatagtggctcatacctttgatcccagcacttcaggaggccaaggcaggtggattgcttgacccagactaggcaacatggtgaaacgccgcctctacaaaaaatacaaaacttagccaggcatggtggtgcgtgcctgtggtcacagccactcgagaggctgaggtgggaggatcacttgagcctgggaggtggaggtcgtgttgagccaagatcgcaccactgcactccagcctgggcaacagagtgagacctgtctcaaaaaaaccaaaatccagaaaagaacttatatggctgcagaggtataatcactaaggaaatttccttttgtataatcttttttcttttactatcatttaaaaaaatgtgttatatttctgaagcaacacatccaggttctgcacatagcagccaaagtgaccttaaagaatataactgggtcttgtcattcccttatttaaactcttgtacccatttcccagtgccgtttagatagagattccagactcgtcaatggctctgtcacctcagacaccctgcattgactcattagtctgattagagtcaggtttttcttcctcctgatggtttttttttcccccttagttctcagcggaacagtcacttccttagggaggtttccccagccaccctctgaggccgtgcttgttgccagactctgccactagagggcagggctgcaccactcctggcacctcgcacccggcctgccctgtcactctgtgtgttgggtgaattcctgtgatctgtgactcactgctctgtgtcctacacattcggcttttcttctctccccacaaccccattttataattctcctttttcaggaaagctttattcccatttaaaaatttttgtttttaaaatggtattttcttacacttattttctaattaaaaatgagtgttttaagaagtattatgatttactgcaaataatttttaaacccagccttttagatcctctgtgatcataagagaaatgaaggatgtctcccaacacttgagcttcatccacatttcatcctcctgttctttcagctgagttttccccatcccattagggactgttggaatataaaactggcttttccctaacagggaatgaattgcttctgtttctcctgaaggagagctggaagaatgacttgcgttcttttgcatacacaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggtatgattgttgcctcaggtcacaaacatgcgagtgatgctgtgagtgagtctgtggagggtgagggcttctgaacagggagtcctgtgggagtgcttcttggggtatgttgtatgtcgtaatttagactaccatcatttgtgttatttttgaggcacctaaggacttctttccacttctcatttcttactgtggggtgaagagttgaattgggagatggtttctagatgcaaattgaaaaggcatttttccagagcagatttgttttcggcgtactagagtgactctttaacctagctgcgggaagatgactgtgccaagactgcaggtaggagaaagctcactgacgaggccttgtgggtctgaacgtcctgcagctatcagagcctgttggcttcctgttgtgcattccaacaaatcatcttcaaacccactttagtgttttgtttataatgtccagaaatagtgaccctgtcacatgctctacagattacaggattcttagcctcttcctttttggtaggtcagtcctgggtttgagcccaagtgaccctcctgggaggtgatgatacacactgggtagagtggaatcagatggacttggattagaattctgtcctctttactagttattttcctctaggcaaactgcccaacagctctaagctatttccttcgtattctgaaaaataagccttaatgggacccatatagggcaactctgagagtaaaataaaggaatatgtgttagagtgtagcatagtcacccacgggaagggcttagatgttagctgctactgctcttattagctgaatgatttggaataaactgttagcctctctcatgttttttctcttgagcttcgaagttttcttgttaatactaaggagatattcaaactagtcatggggttttggaatgacgaagggagatgatgaatctaaagaatttagtgtaatatttcttcatgctcagtaaatggtagtttctgctgctgttatttttattaccatctctttggaatgggagtaggtgctcctttgtggtcagaggctgtgagagctccacagcgccagtttgcccatctgtacactggggtctgttgaaggcagtcccctctgtgatatctctggctgtcagagctcagatgatagatggtatttttgtactcttagttctcatcattttcatgatttcgatcaccatttgagtatgatgatgctaacactttgttgaacgtagaatccgttaattacttccttcctgaacctttggcattaaaaaaaatctattctgctacctctctgctcatttatggttattcaaatttattatcaagagcctggtacagtggcttgtgcctataattgtagctacttgggaggctgaggtaggaggattgcttgaggccaggagtttgagaccagcctgggcaagatagtgagaccctatctctaaaaaaactgaaaaaaaattagctggacatgatggcatgtgcctgtggtcctagctactcaggaggctgagacaggaggctcggttgagcccaggagttggagttcgaggctacactgagctgtgattgtgccaccacactccagcatgggtggtaaaacaagatgccatttcttaaaaaaaaaaaatatatatatatatattatcaatgaaattcagtagtaccaacaggattataaacaaagatagtagttcccttcctactttttctcttaatccttgtgtctcacaggcaaacataactcttagtatttcttccaatatttactttcatgtttctttctttctttctttttttttctttgagatggagttttgctcttgttgccaaggctggagtgcaatgacgcaatcttggctcaccacaacctctgtctcccgggttcaagcgattctcctgcctcagcctcctagtagctgggattacaggcatgcatcaccacgctcggctaattttgtacttttagtagagatggggtttctccgggttggtcaggctggtctcgaactcctgacctcaggtgatcctcccacctcagcctcccaaagtgctgggattacaggcgtgagccactgcgcccagcaacttccacatttctaaataacatgcttctactgctattttttttttcaattttagacatttttttactttcactatagttctatcagaattcagtgtgtacgttattatgcctaagtaaatagtcatggttgcttacgtattatatttctttgattgtgtttcttatttgatgagaaagctgtgttttttgctctgggttgaaactggagagaggacctggggaggaggaggaggacagatgaagttggtgactgtaccttcatggccatagctgggttctcagcacccggggatctgctgatcacctactcataggccaggcccctatcgaagttctaggtgacccagtgctggggacgggggggccacctgcaaggtctaatcatggaggtgggggctacagtgttggcttgtgctggggccagcatccttaggaaggcatcttggaggtggaggagacagccgcccacttcttgattggggccttcagcagcaccagcttcttgggcaggctggtgctggctttcatcaccatgtcgtgttcaatcttcttccagatcctgacttctaggttcagctttcctcagaccctggttcctttcagaggccattgctgctgccttgctctttgctggcttgtgccttgattatatgtctttgtacaactttttgttttcctggagttaatcttcacatctgttttcttggagttaatcgttacctctatatcgcttgcttattattctttggcctttttgtcttctcacaccttccaacttctttgtaatatgtgtttagtacaatttttcatgacaggtagtttactgaatcagtttttccccagtgtggtcatccaacttgagttatccagctctctgccccagtctgggcaggttgatcttcaggtctgtagtacacttgtatcctaggacttctctttgccattagcctggaatttcctttgcagttctcccgttggatgcccagttcctagatgccatatgtttttctatcgtctagtagcttcctgagagaagatgaatgggagggaaattgtatgaggttttgcattcataaaaatgccattttttttcctgtacacttggctgggtatggtgttctggggtagaaatcattttccctcagaaatgcaaagtctttgccctgttgtcttaaaatctccaacgtgacccgattccttaacctatgaatgtacttttctttggaagctttccatttttggggaggtgaagtgctaggtacttagtaggccttttaatttggaaacttacatcccttcagttctgggaaaattttcttaacatttctctgagaagttcttgccttttattttctgtgttctctcctgaaattggttagttggatgttggtcctcctagattgactcacatcttacctttttcttttctttttctggtactttttagatatccatctcaaactcttctattcattgttatgtttttaacttctttcttttctttgtctcttgatggggtcttgccctgttgcccaggttgtggtgcagtggtgcgatcatagctcactgcagcctcaaattcctgggctcaagcagctgttctgcctcaccctcccaagtagttgggactacaggtatgcaccaccacgtccagctattttctttacttttttttttttttttttgagatggagtcctactctgtcgcccaggctagagtgcggtggtgggattttggctcacttaagcctctgcctcccaggttcaagcagttctcctgcctcagcctctcaagtagctgggattacaggtgtgcaccaccatgcccggctaatttttgtatttttagtagagccagagtttcaccatgttggccaggctggtctcgaacgcctgacctcaggtgatccgcctgccttggcctccgaaagtgccgggattacaggcgtgagcccatcattagatctttaaataccagtatctataagtcttttcctcttgagtcagctagtatccctggaaggaaattactcattttcctgcttggaggctataagcttggctatgtttatcctgcaaccggggactggaagggaggggactgacagtgttgctggtcagggtgccctcttactttttgttttctgtgtgcatctcacgtctgtcctcagcctatgtaaacacctcttgagattatccctctcaatctttgccggaggtgggggaggggctgcttcctgggctgccttggattggagggaagacctcaggtgagtgggtgggaatttgcccaaggagccatgagaccagccactatttcaccctctccatccctccactttcagatgtatgtggcgcctccaaagcccgagctcttcttggcgtctgtggcttcaataagcttgctttttgctggtatccctcctaccctcccctgtccccagcaaagcttgcatttgaacttcttcctacgggctaacaaatcagtcagttatgtagctcttgttactttttagcttccgaagttttgttgacacccgtagtctgctaatgtccctgttctgttctttctgttcgtgtaaatatatgctttatacaacttctttacatgatttttgtggggtttctgggtagcagagcttcacaagttcaatccagcgtgttggattagaaatctcccaccctctggtttattcttattctcaaaattacctgccaaacactgatactcccttgtttttccttttcctgacaggaaatgtacataccatacaggacagaaatcattagtgtatcccttggtgaataaccacaaagtgaacttaacccttgtaaccgccacccaggtcaagacagaatattaccaagcactcagaagcctctcccctattcccccgtcactgctcctgccttcctccccaaggtcatgactgctggcttctaattccagagtctgtttttaaattctgtgtacatagaccatggattaagtgttctttttgtctggtttattttggtcgacattaagttcatgagagtcttctatattatcgtgtgtattagtattcctgtagttttaggagcttcatagcattccattgtagggatataccacagtttattcattgtattatcactgggttgtttctagttcttggctattgcgagcagtgctactgtgaccactcttaggtgtgtcttttggagtacatgtgcaggtttccatcttgcacagctagaggtggagttgttgggtgatagggtgtgtgcatctcagctgcagtagaaactgccaaatagctttccttgagtgcttgtaccagctcacccttttgccactgtgtatggggattccaggagctctggtcctcgctagcacttggaattgctgatgcttttactcttagccttcctgatgggtgttttctggaatcacattatgattttaatttccattccttaaagtacccttggctctgaagtttaatgattcatgcatctcttcccttttgaagtactcttacaggtatgttgtgcatgtgttgaaaagtggcactatctattctaaaatacagtatgcctcctctgtgtttgaacagttgtagcgtggccttggggcctcctgttagctggcttggagaagggattcttgggattgtagagattagacctgaggaggccccttggagctctctgactaaattttattctttattattccaaactatttaagctcaccgtgtgctgactcatcataataatgagtagctctcattgtgcttgtctatttggactcatacaatgattttttttttttctttgagacagagtcttgctctgttgcctaggctggagtgcagtggcacaatctcggctcactgcagcctccacctcccaggttcaagtgattcttgtgcctcagcttctcaagtagctgagactgcaggtgcgtaccaccatgcctggctaatgtttgtatttttagtagagacggggtttcaccatgttggccaggttggtctcaaactcctgacctcaagtgatctgccttcttcagcctcccaaagtgctgggattacaggtgtgagccactgagcttggccaaagtagttttttaagatgttagtatcttttcttgcagctaaaaaagtttgtcagagatgattctactttgttctccaggtgttttctcagggagaaattggaggcagtaagccactgggggagtcctgtggctggggggtggggtagtcctgtggctccttgtcagggagtcctgtggctggcaaggagagaagtcctgtggctgggttgggagggagtcctgtggctggggtctcatcctgtgcctaacagtgtccagaggtgccgagaccagctcagtcggggagaccctaacccagcagcgctagaggaattaaagacacacacacagaaatatagaggtgtgaagtgggaaatcaggggtctcacagcctttagagctgagagccctgaacagagatttacccacatatttattaatagcaaaccagtcattagcattgtttctatagatgttaaattaactaaaagtatcccttatgggaaacgaggggatgggccgaattaaaagaagaggttgggctagttaaccgcagcaggagcatgtccttaaggcacagatcgctcatgctattgtttgtggcttaagaatgcctttaagcggttttccaccctgggtgggccaggtgttccttgccctcattcctgtcaacccacaaccttccagtgtgggcattagggccattatgaacatgttacagtgcttcagagattttgtttatggccagttttggggccagtttatggccagattttggggggcctgctcccaatacagaggtctcgtgtaaattccctgggaggcgataagcctctgagaaacagactatgctaaccacgccatgaaagagaaacttatttataaatcagatgccagttactagtttactgcttatttgcccaggcgtagctctgacagagtccccgactcatagtgcttgctcagtgcatgctgaacaatgattggaatcaagtcatggctcagagcatagttttgaataatgggaaatggatgttcttaagtaacatagtcaccaagataatgcgactagctgggtcaccccttttcaattttaggatatttttatcaagatttaaatggccatcattagagttatagcactttctcctttggattgtcctagaggcccatgagaaagtattccctaatttcttaggagaacagtttgtgggtagtatgcggtcatgtccagttaaattgcagatatttccgatcgaagatgttccagtcctgagaacttcgtgacattagcaggacttctacaagccatctcttagggtggggcatttactgcagttggctagtactcttttctccttaactttgtcatttgttgatttttttttaactgtccccaaatactgtgggcagagtgtatctagaattgaggcctccaccattgcggagaggacatggatgctgagcagtcccctgagtgaaggttataaagaagcaaatagactacacatgtctgtaaactgctcttgagtgtcccaaatttggggtacttcagttcagctgtaggaaaagcctcaaactgtttatactttgcaagaattggaaacttctaattcacgttaagttttatgtaatacatgataagcttcataggagcttcatcttttatctacttggacttttgcttccgtaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggtaaggtggaggcatatgagtggaagagtctccagcatgtactcaagatagacctttgaaataaataaaaccagatgatccctcagcttctagaccaggctatttggcactggttgattgaatgtgaactgcactggggctgctgtgagcccgcatgggtctctgtgaccctgcagatgcagccgtgcccagggactgggcagtgggtgtgggctggtgtgagccctgtctgccacccagggcctggccctctgtctgtgtcggccatgactatggtgagtcttgtaggcttgagactgtgcctcgggttcctgcgggttctctgtaggtcagttgacagtttctcctgttgtttgggtaactgtggaaacgaacactggcaagtgctgaagcgagcatgtggacgtgcgatatgaaataacgacctggctttcaaaggcagtgaggctctctggaaaggaccttgctgagctagggatgtgggtgtgtagccattcccagtgggcctcatggcgtactcgttcatgatcatgtttgtgccatcttgatctctcaggatctcttcttttttaacagattaagccgggaatctccaaacagtgagtcagatgttaagatgtcttgcttccacccccacaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtaggagcacagggtttactctaggccctgcatgtgaatgactgacattcaaagaaccgattaatttggaagagaagcggcagaaccgagagttagaggtgtggactctggagctgcgctgctcgtttccaaccctaggtgctgacctctagctgtcttccctctgtatgtccctgtcaccgtgagtcaaatgcgggtgatgcctcctcaggtgccgtgttacctaagcctctcagagaccactgctaccctgtttctaaaaccagaggtcacgatatgtgttcatccacccagtaaatactgattgagcacccactgtgtgctaggctctgggataggggctgggtatacaatggtgagtatttcagctgcagcttctgccccgtggaggctgtggcctagcacactggtctaggcacggtggtatatgctcactcaaggagatagggacgtggtcgtttggggtgtcggaacaaaatgtcggaacttctctttccaatgcagagaaaccttgcagtaattctaatgtactgtgattggcagttgacttcagttctttgtagcacgcttactcaggttatttcactaactatgtaaccatgcagcctcattttaagcaattggattttttgaactttacttaaaatgttatgtcagggtttttattgtgcttaatgtgtgccatttagctaagttttgtaggatacgaaattgtaagtggcttaaaatgattcttaatagaatcatgaattgaagataatgctaataatttaagcactgagttaggtagtgtttgtaaaatgcttagaatgcttcctggcacatgttaaggccatgtaagtgctgcgtgttgataaacagctgagcaaaagtggactcttaagaaagtattggggctgagagttctgttccaaccagctgccctttggttatttttcagaataaaagcagagtctcatgggatatgacatttatatttccttcacaaaaaacactgctgagtgttttgttgagtaaaaagggtgtagccatggtaataatacatttaaaatatagtttatttcatctttaccttgccttgttttttttttaagctagctttttattgagaattccacacatacaaaagtatcaactcatgaccagttatatttcatttataatcctacttctccctttttttattatttgaaagcaaaccccaattatcctcttatttcatctataagtatttcagtatctctatagatgaggactcttctttatttttaaaactttatttttaaaatgatggtcagatgcagtgttcatgcctgtaatcccagaactttgggaggccaagctgggcggatcacttgaacctgggagtttgagaccagcccgggaaacatggcgaaaccccatgtcttaaagaaaaaaatcagccaagtgtggtgatgcatgcctgtagtcccagctacttgggaggctgagatgggagggtcacatgagcctggaagatcaaggctgcagtgatccatgattgtaccactgcactccatcctgggtgatggagcaagattctgtctcaaaaaaacaaaactgcaaaacaacgtcacaaaacagtgccattgttagacctgaaaatattaaacatttcctacatcaaatacccaccaactcattatcaatttttctctctactcttttggaatcagcatctaaataaaattggtcgataaggattgtaaatctctttgatgaactggttcccctccatcccagtttttttcccttagagttcatttattgagaaaccagattgtttgtcttctaagttttcctgtggtctgatatactgcttccatctccactgtgtaaattaacacctttttctcttctctgtatttcctgtaaatcaataattggaggaaaagccttgtcagatttagtgtatattttatatctgagtccagtatttcttatataatattttaagataagtgtactcttttaaaaagtattgaaactatatgctcaattttttttaactgatgcttttaagaaggctgcttgatcataaaagtttagagatcattggtctgatgggaaaagcaaataattactaaaccgtttagcaaggttgaggtgcacatggtggggcctggagaagttcagtcatgagccgtcacttatgggcacgtggaatctgacccggcacagagttgggagaagacaggagctttatagacagaaaatgtggtctttgctaagtcccaggagtgaaagggtgagacagtgctcacagcacacgagtgtgggtgcgtagacagagcaagggtgggtcctgaaaaggcctgcaggctttctcatagattagcaagagtgctggttacggaggtttctaacatttgtgaacagatcgaaactgtgttaaattgggattgcagtaatcctggaaggacagggatagagggtgaaggggaaaaaagggtatggatgtgagacttaattgctgattttcttaagacctttctccaaagtaaataaatgatgtggcacatttttgaactggcaaattctaaactctagatatgattatctctataacatatcttactccatcttcttttgactaaaaactgttcttaattaaattaccatgagacgttcaattcagcaaatgtagtttggctaaccatatttaattagaatttaatataatcctaggcctggccaaactattaagcaagtgtgggcaaaatattgataattttagatatgcaggaacttagtttgctttccatgtgtgcttttcgaaaaaggaataaattgaaaaatagaggaagccctgaaatccaagaagcaaactctctcacctaggcatgcagtaaaagcaattctaggatgattgctgtttggcgcgtagttcgtattagaaaccattcttcttgaataaatagtatgtttaagaagctgggcagagggaaggcatatgcatatattatcaacaaggagggagaaaaaggcaattagtaaccatccataggagggtcagcaagatttataaaggaaatttgtgatccaagtatgaagcaaaataaggtgcagaataaattttaagcaagtaatagattagagtaagagaacccatttgaccattaaccttgggacattctctttcaaatgacatggagtagtactgaaatctttctttctttctgagtctaggttattgtgactggactcagaaagaaatatttcattattgcagtgaataacatttgtgaacattattgttcataaattatgcagtgaataacatttatgaacacgtgatgtgtaagatacatactgtttatttttagttaagttttttggctcaacttctaggcagagaacattaaatgtaaatagtgttacctaggagcatgtaaatggaaatctccatagtatgaaagcagtgctgttgctaacagaatttaggagggggcagatgaggtgaaggaaatgtgggtgctgatttccttattacattgagaggagccaggagattctttgttcaaaatggatggcttaagaagtcaaagtataagctgattacgtagagcaggtacccaaaaatgttttgtgtaaggggccagatagtaaatattttcagtcttgcaggccatcccaagtctgtggcagctactcaacactacctttgtagcatgaaagcagccacaggcagcccataaatgtggctctgttccggtgaaactttaggtacaaaagcaggtgcaggccagacctgacctgtgcactgtggtttgctgacctgggattcaggggtatagaagttaccatcagaagagctaaaagtgagactttttactttatactcttctacactgtctgattttgaaaaaaagaaacatgtattttataatattaaagatagggttggcaaatagcaaataaaaatacagaataccagtgaaatttgaacttcagatacattatgagtaattttatggtgtaagtatattccaaatcatgtgggacatacttacactacaaaattatttgttgtttgtttacagtttaaatttgagtgccttgtattttatctggcaactgtaattaaagggaaaaagaataaattcattatgttcatataatgtgatatagcaggggtccccaacccccaggctgcagagtggtactggtccatgggtccccaacccccaggctgcagagcggtattggtccatggcctgttaggaaccaggctgcccagcaggaagtgagcagcaggtgagctggcattcccacctgagcaccgcctcctgtcagatcagtggcagcattagattcccataggagtgcaaaccctattgtgaactgcacatgtgaggggtctaggttgtgcgctccttatgagaatctaatgcctgatgatctgaggtggaacagtctcgtcttgaaaccatcccctggccctgtggaaaaattgtctcccatgaaaccagtctctggtgccagaaaggttgggtagcactgtgatatagtattaaaagtgctaataaatatggcatactgcctttaaaatgtctggtagctctttctcagtggcactcataatagtgttttttgatttttaaatgtgtgtcaagctgactctcccctccgtgtatgctgggctttattttccctttcctagtcaccagttttgggaaatagagatcttcattctcatgctgctcctctagtgcaagtgctccatttatttttaaggaattaatataacaaaaaatcatgggaatttagaaaacaacatggaagctaatgatcacattggtggaagtgatagggaaatatttagggggagaagttaaggtataaactttgtcaatgaagtcctattaaaaacaacaaaaaagtgaagcttaggatgcattttataaactctgaccagaacacctgtgtttctctgtttctaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttataggcaagttattagcaaggtctactcttacaattaactttgcagtaatactagttacactctattgattatgggcctgccctgtgctaagcagtctgcattccatcttccttgccaaaacttataatacaaatttcatctttattttataaataggggagttgggctgggtgtggtggctcacgcctgtaatttcagcactttggaaggatcgcttcagcccaggagtttgagacaacctggccaagtgagaccctgtctctacaaaaaaaaaaaaaaaaaaaaaattagctgggcatggtggcacatgcctgtagtcccagctgctttggaggctgaggtggtaggattgcttaagcccaagaggttgaggctgcagtgaatcttgatggcagctgcactgagcctggtgacagagcaagatgctgtctcaaaataaatttaaaaataaaataagagaattaaagtttagcaggttgggtggcaaaatgaggccacacatttaaagcccctcctcctgattcttttctctgccttggctgcctcctgtggcattttaggtgctgagaaatgaaaacagtagggaaaatagttccaggatcctcatgttaatttgccagaaatggcatcttcaagtcgtcagagggatctgagagttccttcctggcctgacttgagaaaatccgtctgtccccagctctgcgtctgcctccactgcccagtcacctcctctccatgctcttggggctgggccctaccccaccatgcagtgctgccctggagcagtgagcttggtgggtcctgtctggcatgagagctgcctttgggagctggatcccagcctctaccactgggtctggtgcctagcaggctatggataaacttctgctgactccggcctctcctaagccactgcaacgtggtcggtgtagtgcacagtgtgtgtgcagcgtggccttactcacagcctccacattagagagaatctgactgaagtcttactgctgcctcgtgtgaacataaatgtttgccagaaccatgagcaggaaatgttaatctgccttgtttcctgtcctttacacggaagaatttttttctgtatggaatgcgtgccttacaaataatgagtggaaatacccatcgctaatgaaaagttatacttgactgttagtcagctaaataatctgagatttctaatacttttaatttggcttttacaatgcaatttatcttagcttttttgatttcttaggtcatatctttagaactatatatttgaatgttaatgtaattttcatattgaaattaaaatgttgaactgcgatgttaagtgtttcctgtggaaaaacgttcacattttctctagttttaaagttgaatcaagctgtttgaagattttcacatttcttctagattttatcagcttgttactttatctgtcactttctgtgatttgcagctggagggggttcctcatgcagccctgtcctttcaagaaaacaaaaaggtgattatttcagaaatcagagtcttgtgttgaatcttactgattttcttgtatttctgtaatgtaatgtatcttgtatttcttgtaatactgtattggactctgtgtatatctcttctcagatgagtgattatatgtgtgaatgttgctggaatctgataaccaggcctgaatagttttgtagggtggcttttaaaaattactttcatatcagaattgctttgtcataaattttgaacgcatcataaatttctaatgttcggggtcagcagactttttttgtaaagggacagagtgtaaacatcttagctttatgggccatatggtctcttttgcaacattcagctctgccctgtgacaggaatgcagttgtaaagacatgagctactggccagctatgttccagtagaactttacttacagaaacagacaggctgtagtttgccaatacctgccttagggaatgtgttgttatattttgtgagttaccttctcagtaaattttatttagtattagtcaggaatattattaagtagcttcttttccagcctggtcaacatagtgagacccggtctctaccaaaacaaaacaaaacaaaaaaacagccacgcatgtggcatgtgcctgtagcctcagctgctgctcagggggctgaggcaagaggattgtttgagcccaggagtttgaggtcacagtgagctgtagtcatgccactgcactccagcctaggcaacagaatgagaccttgtgtcttaaaaaaaaaaagtttcctttgttgggttattttaatttggacctggttatcatttttcagccatatttaactttgtacatatcagaatgttctgataaaacttaacttttattaaagtgtttgtgatataatctgctagttttggtacacattatcttttgcaatgccagttattttcttttccagtgtgggtttgcataggaaaagaattgctgtcactttctattttgaaatcttaaaagactgatccttttttgtgtcatgatttgagtatttaattgagagcctaatgcctaatattatttgcagtattaaatgggatcttaacaggaatagcattctagccttcattgaattaagtaaacatttcttaagagaacttggaatctataatatttgcgtcatcatagtatgagatacttaatcaagtttgagattttagtgaaacattgtttagaagccaaaaggattctaggaaaaattaatgtctatattcttgaattaggagagattttgggacgtgtgactaagttacgctgacacttgtttgtttcttagtcgctttttccagtggcggtgagaacgaagatgactgattcacattgctcagatgagtttatcctcttctggctgggacatgggatatatcctgtctcttttaagcctttttggtatttttcccccattgagagctgtgtcttcaaactcttctgttatagctggaaaatcctttttaagtgaaatctgcccaaattataagacagatgaaggtagagttgtgttggatataggattagggtgaaagtagtgggggtgtcctggagcctctcttctggtggcagcctagctcttgtgcctttgaggaaattaccctggggacggctctgtggaacatatttgcaaaccactgatttggaagatagagatggcttttgttaagatctgaattcacctttttggcattttatttgatttctcaaggtaaagaacttattttgtaataaagtttcctattatttagtagataggccaagttgctgtgttaattccatgtagattttgggtttcctttgctcattttttcactcttaatctcacatcattgtaagtttatggaagttatcatacttctgactttttctttgaagagcagaaattagaaattcccaataattattttgatagtgtcatttaatgacactcacatgtgatgtagccacaaagatttaatgagttcagttttaaatcatattaagactgttggtttcatttgttctcattaatgtaattctgaagatgaacaataaaatgtatttttagaactttcaaatgaaatattatttcatccttccagatcatataatgcttaagttctgattgttaatcataaagtctagaaaattaaaagataataaaatgaaagtgacttttaggtattagagttttattataaattctggtgtgtcattggagctatgacatgaatatttcaaaggccaatagcattggatctttacagttataacttaccatttttaagtttaagtagtaatatagattatttaataatcaaaatcaataaatattaattattaaaatgttttgtggtatagtttgagaatcattgcttttaactttttccatataggtttattgactttaatagcattctaaacataacatctctacattctttgtgtttaatactgtggaggtataaaaatacttatatatgatgataaactatattagagtaaattaaatattcttatgagtttcattttagagtgcatttacttaattttgaagtccttatttttagcaaactaaaaggaatgttggtacattatttactaggcaaagtgctcttaggagaagaagaagccttggaggatgactctgaatcgagatcggatgtcagcagctctgccttaacaggtagttctcactagttagccgctggtgtggaccttcactgtctgccttccaccccttgcccttcctgctcgtccccctgcacctggtggacagcacgactgggggcagcagtggagccaggttgcttaaatggggcatattcgggcttcttttataatacttactctgaagcttgtgtgtctgtggtgtttgcatcatatatttgttgttttccatggtttaggctgttttaaaattaggtttatggcttgagcatagggctttgtgagtaggggatggcaggtcgaaacatctcatgagttggatgggttatgctgggggttgggaaatgggatgaaaaattatgggatgaaaaattgcctatggatagtttaacttgaaagaatctgcctttgtttacagatagttatcttttttcttttttgagatagagtctcacactgtcacccagtgcagatacccagtgtcactggagtgcagtggtgtgctcttggtgcactgcagcctccgccttctgggttccagcgattctcctgcctcagcctcccaagtagctgggactacaggtgcccgccaccacgcttggctaatttttgtatttttttgtggagacgggtttttgccatgttggtcaggctggtcttgaactcctgacctcaagtgatctgcctgcctcagcctcccacagtgccgggattacaggagtgagccactgtgcccggccagttacagatacttatctaatgaaattctctgtgtactttataaaagatgaggattaactgaaggtactaataactggattatatgagggtggttttggttgtataatcctatctaaaagaatattttagctataactgaaagtaagacttaaatatttagagaggaaaatctgaataattctagtagtaattatttatttacaaaataaaaatagatttttttttgattacacaaattaaacaacaataaaacatcacagcaatccggatactataaagctcacatgcttaccgacccaactgccccaggagtgaccactgccaacagcttcatgtcgacctttttgccataatttttatatagccttttttgtttttaaatggtaatttagaaagtcaactaggaaaatgtgttacaggtttatcttccaggagaataggactggagtcgagatcttgaatgtggcttggaagaaggcaagcccaccccagagagatgagttgacagttgtttctgaccactgcttgcttagagggcctgcgtgtctgtgaccgcctagctttgcgcccctgactaggctgccccttaattacaaatgtctttatatattgctccagctaaggcttggagtagtcggttaagaacttgaacttcggtttttgcagtgaaacagcatttgagaatatcaccttctgataagccttattttataaggtgggtactgtagtgggaggcagtgtgagagatgcttgaaggatgcactgctgtcctgcatttcagcatcttcaggatgctgtgcagctgaaacatttgataacggtggaactgttcgttattttgcaagcctgtgattccctattgaatgttttctctcgccatttgacaaatgagtgtttctctgtcttcagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtaagtgggcagaggggcctgacatctttttttttattttttatttgagacagagtctcactccatagtgcagtggaggccgggcacaggggctcatgcctgtaatcccagcactttgggagactgaggcaggcggatcacttgaggtcaggagttcgagaccagcctggccaacatggtgaaaccctgtctctactaaaaatacaaaaattagttgggcgtggtggcacatgtctgtagtcccagctgttagggaggctgaggcaggagaattgcttgagcctgggaggcagaggttgcaatgagccgagatcgtgacactgcactccagcccgggcaacagagcaagactccatttcaaaaaaaataaaaaaataaagtgcagtggctcgttctcagcccactgcaacttctgcctcccaggctcgagcgattctcccgcctcagcctcctgagtaggtgggattacaggtgggcaccaccacactcagctaatgtttgtattttcagtagagacagggtttcaccatgttggccaggctggtctcaaactcctgaccttagatgatccacccaccttggcctcctaaagtattgggattatagttgtgagccaccatgcccggccctgccacctgccatcttttgagttcttccctggagacctagacctgaaccctcctgcttgttctcttgttatctaatacccctattgacagcgcagcttagatcattaatggagagcttgacctcatctgataccttcactgaaggaaacaacttagtgtcttttgtgttgaacactgaggtaaaaaattggaatagttgattatatgaactctgctaaaattgagtgcattttacattttttaaggccttgttgggccctggttaaataattatttttaaaaatccttaaggagcctattataaacagatctgtggtcttaatgaaatgtgattaatactgtgcattattttaagaacttttgacttttcaaaaaacttttacaacatttcccatttgatagcggcataggtttaagcacttctcatctctaagttagtggacaaaaaaccctcatggatagtctaataatgtttgctacaagtccatgttgagttttatactccattttattttcagttttaaaaactgtggttaaatatgtgtaacataaaatttatgttcttaaccattttttgcgtatacagttcgctggtattaaatacatttaaataatgtcatggaatcattgctaccacccatctctgtaaccttttgatcatgtaacactgaagctctgttcccattgaactctattcctcctttcccgccaagtccctggcaaccacgattcttctttctgtcttctgaatttgactactttgggttctcatatactttaggagtcacacagtatttgttttacttagcataatgtccccaaagctcatgcatgttgtagcctatgttagaacttcctaatgtttcaggccaaatactattccattgtatggataggccacattttgcttttccattcctctgtccatggacacttgtattgcttcatgttttagccattgtgaatcatgctgttatgaacgtgggtgtacagatagctcctggagactctgctttccatttttttggctaaatacccagaaatggagttgcttttacattccaattttaatttaaaacattcatatcattgagtgttttacttaatagtatagtagttaacaaacttaataaaatagtattttggtaataatttgctggtagtccattgttcagtttttttaggtaaattacacaggacatttcaagtggacatgaaacatcttgtgatgtggaatcatgccccaagctgatggctaaacatatgaaataccataccctaaatttagtagatttagtctttgcaatttaggagataacctgttatattgttaggtttttgtcgaaaagctttgtcctcatatttccaacttgctgtaaaatttgtttgtgaagacaaatatttttgtatgggttttttctttttcatattaaaaagaaatgtccacattggaatttttttggagtttttagagctaatagagcttttcataatgtagtgggaatgagtgatcagtaagctcttagcagtttccatgcgtgcatttctgtgccttgaaataaatgacagatgagtacatttgtgttctgtgtgtaaaatgtgctctttcctcattgcacttccatgttggagggcttgtctcttggtgatcacacttcaaaattctcacagccccccttgaaccgtttaggtgttagacggtaccgacaaccagtatttgggcctgcagattggacagccccaggatgaagatgaggaagccacaggtattcttcctgatgaagcctcggaggccttcaggaactcttccatgggtatgtggactacaggtgatgcgctacaaagtggtttgtattcagacctggacatcttaattatatctttgcttccaagaagaagtcctttgatactgttttctgagttctgaatagctgatgaaaatgaccaattgaggaataatcatactttttcttgatctaaatcttatacttttgagttatcttagcataaatgtataattgtattttaagtggaaatttgtcacttaatcttgatttctctgtttttaaagcccttcaacaggcacatttattgaaaaacatgagtcactgcaggcagccttctgacagcagtgttgataaatttgtgttgagagatgaagctactgaaccgggtgatcaagaaaacaaggtgagggacataggcttgagacgacttggtgtttctgagcttgtgtgaggatttaaaatcgccctggctactgtctactttattgctttcccatccctgggcctttaaatttcccctttaaataccagctcttcccaggcctgttgttttctgcctttccaggtactacccacagccttgagaattgcctgagttctgcctcctttgagagtgtgccccagacaaatctattctgtactgaatgtttccttgtctgatttcttggatcattcatttgatggttgcgtatggcctgcaacgtttcttgttttggttctactgaactgttctaaaagtctctcttcatattatctttttacatgtaaatgtaactgtcttcacttttaattcctcaaggacaaggaatagcgtttcacagttcgtcccatcaatcagaattatagcctttggcatctccctatctaccaggcccacttcctcttagatttgggcttccccaggctgttgcctttccccaagtagcttctgcttgtcctgtagaagacctttcatgctttgcttctgcagcagccgttcctgaatgcctagtgtcaactgccttcttaccacgcccaccctccctgcatgctgcatttatcccctgccacagccctgtgaccctgtgtcctgctgcctctgacttgtctgtttctgcttggccatggtctctgtgaggtcaggtgtgcatatgggcacaaaccagggcatctctttatccccagcacctggcttaagtgctgctctggaactatctgttgaatgaactaatgcatgaatgtattgttgagtatgagacaaacaagtgtcattgtctcctttctagccttgccgcatcaaaggtgacattggacagtccactgatgatgactctgcacctcttgtccattgtgtccgccttttatctgcttcgtttttgctaacagggggaaaaaatggtgagtacaaaaggggatgtgcacagttgaaggaaataactaggtttcagaggtcagcttggtggcctgtttttgccttgcgtgcagcagaggaagtagaatctgaggatgagtttggttttcactagccgaggggagggaggaaatgatgggagcaggtaggttattgggtctggttttgttcatttgaaaacaatctgttgtttgaggctgaaggtggcttgggtgatttcttggcagtgctggttccggacagggatgtgagggtcagcgtgaaggccctggccctcagctgtgtgggagcagctgtggccctccacccggaatctttcttcagcaaactctataaagttcctcttgacaccacggaataccctggtatgttaaaagttcacatcttattttctcagatttaatcattattgtaaaaactatttcagtattgactattttagttttagagcagtaagtgttttgagttcatttgggatatttgacctgcgttgtagctcttcagaaaacacatgaatagtgaagttctttgtttcatgggttccctttagatgaaacccatagaggagaaaagtagaaacctcagcacgtaagagccaacatatatacacatcggatttaaacctaaagcacaaattgtgcctggtcgcagtggcgctgagtcgcactcagccaggccaggcattcacactcagggtgagtgggaaccaggactggctgaggcagcagtggacccaagtctccatcgcgcccatgcttactatggagccttctcgttctctctttttctttgggtgagagggtacacttgtgtttttgaatttatatgaggtaagtgtgtaatagggttttttctaatcttttttaagtggaatctggaattttaatcagatttattatctgacaacctagaattataatccagaaagtctgtggtattgaggacatattggcaatatgatgaatctctaattcttaaatcctgaaacttttttttttttaatcacttagggttattatagtgaagtcatttctgaatttggatcttctcttcacacctctttttctctttcctgagaattaagcttttgtttcgagttagaaagttgatagtagggaattgttccatggctgagcaatttatctccacagaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggtaacggccagtttttcagctgtgttttttctagttatgcttactaaggtttaagtttagatgatgatgtttgttgcttgttcttctggttaggaaatacattttctttggcggattgcattcctttgctgcggaaaacactgaaggatgagtcttctgttacttgcaagttagcttgtacagctgtgagggtgagcataatcttctgtggaaccatttcttcacttagtggacattttatcattgctacaattaaaattggagcttaataggaaatatttccatgcactctaaagctgtaaccagtaatacccaccatgtatccatctctcagctttagaaagaaaacgttgccagtaaagttaatgcttcataaacttcagtttaagttctaattctcagaatatttgtttgaaatagacctcttcctaaaggatatatttagaaataacctatcattaagtgtaaagtctgttgaatatgctgggcacggtgactcacacctgtaatctgaccactttgggaggccaaggtggaaggattgcttgagcccaggagttcaagactatgggcaacatagttgaccctgtccctacagaaaattaaaaaaaaaaaaaaaaaaagtagctgggtatggtggtgcatacctgtagtctcagctactcgggaagctgaggtggaggggggattgcttgagccccagagatcaaggctgcagtaaggcgtggttacaccactgccctctagcctgggcaacagagtgagactgtctcaaaaataatagtaataataatcagttgaattaaaaaaaaaaaaaaaaaaaccactgtgctaggcccatagtatggtaagagttaaagtgagccttagggattatttactcaacctctgtttctgtataaagtggaataggctcaattctttaagtgatagcatgttgaacctttccataccaactggctcataagtcacaactggccagtcaacaagagtaaaaattaactggtaaaaatcaaagcaaaaaacctacaattgtcaaatttgtgggataactcccccttttaaaatgtcatgcctgacagtaatttctctctagtttccaggttttcagtcagttgtgtcttttttgagcagaaggaagcatgctaagagctcaatcttgtggctagctgggggtctttgtgtcagccatgcatgtgatggtgcccctgggtgcttggggctgcaggggaggggtacagcagtaggggcctgttctgttctctcgtgctgtggagtacatagtgacatagtggggtggtccttggtgtaggtcccttgttcctacccctgggtctgagatttatttagaagtggtgttggggctgtgcggcaggcccctctgtaactgatcaatgtttgtgaagttgctgtttgagagttgaaaccatgacataagcagaaatggaaggaagaaagaaccagttatgtgaaagggacacatttacttttaagcttgtatttactgagataaagtattcttaatcaatgttcttgagaggtgtgggaaaaatgcaacatcctggttgcagttaaacccagaacattgtgtgttgaagagtgacggttctcaaaccgtcaagacgcgggtactgagtgggactaacctgctgtcctcttgccttggaccttgtgttccagaactgtgtcatgagtctctgcagcagcagctacagtgagttaggactgcagctgatcatcgatgtgctgactctgaggaacagttcctattggctggtgaggacagagcttctggaaacccttgcagagattgacttcaggtaagtgagtcacatccattagatttcatgaactaagctcaattgaaagttctgggatcacttgatgcaaggaatgatgttatcaagtaccctgtccatcagaaatccgagtggtttaggtagatgacagtgattttctcctcccagtggctttttgctgaactttgccctatgcttggaattttattttattttattatttatttagagacaagatcttgctctgtcgcccaggcttgaatgcagtagcacaatcatagctcactgaagctttgaactctaggactcaagtggtcctcctgcctcagcctcccgattagctaggagaataggtgtgtgccgtcacactggctaatattttttgtagaaatggggtcttgctatgttgcccaggctggtctcaaactcctgggcttgattgatcctccatcttggcctcccaaagtgctgggattacaggcatgagccactgtgcctggcctagaattttaaaatataagtagaagagtagatttttttttttggtagtcctcgtcatttaagtattctggatagtgggaataaaagagcttagaatttttcatctttgtcttaaacttttaaaaaaatgtagcttatattaattctgcttgtttaaaaagaatatactcttcattatactgaacctaggtaagacagctggtttatattttgttgcaattaaaaaacgtgagctgtggttgcagtgagccaagattgtggccattgcacttcagcctggcaacagagtgagacttggcctcaaaaaaaaaaaaataacatgagctgtgttggcactttcattttctaagagtagttttggctggagaagttttctttcagtactttcttttagaagggaaattttcctttataatttagggtttgttttttttttttccaagccaccttttatagagcccttgtgggttatttcatttaatccttagaatgtttataaatctgggcttgttctcggctccacccacagatagggacgctgagcgtgcatgagtgggcagcaagatagcaggttatggagggcccagctcaccccttctgtggcttgagccaattttatagggcacttacagagtcttttgaaatagtatttattttgaagaaaaagaaaaacagtttactgagtactgtcttattgagtctggaattgtgagaggaatgccacctctatttatttaaagccattggccttttttgttgttttgagtaagtgctgcccaaggtccttccagggcacctggatgagcctgctctggagcaagctggcggtaagtgtttactgagtaactaaatgatttcattgttaaatgtgctcttttgttaggctggtgagctttttggaggcaaaagcagaaaacttacacagaggggctcatcattatacaggggtaagcggtttatttttgtgagatgctgttttaccttcaagaaggtgaaagtgaggctttccttgtggaatttctctaaatgcattcgtcatgttttagatgtttatttcacagtttatatcatgaaagttataatcttgtcatatggatttaagtctagtaatgttgagttctttctcactagctttccaaaatatcttacctaaaatttagtcaaatacaagattatgtttatttttattatccttctctctaaagcttttaaaactgcaagaacgagtgctcaataatgttgtcatccatttgcttggagatgaagaccccagggtgcgacatgttgccgcagcatcactaattaggtatttaccaatattttatctcttttccttttttggttgaagtactaaaagatacgagaatggaaagagagggaagaattcaaaggatgtagagcagtattcctgaatctgagctcatttcagccattctattcttaaactataatgaaaaaaaaatccaaaaaagtctaaaattataattaaaaaaacaacaaaatactaactgtccattgtaaaaagtaatgcactttcattgtaaaaattttggactatagagaatagtactaagaagaaaaaaaaaatcaccttcaattctgctgccacctggaggtaatcactgttaatattttgctatatactctatgagtttcttgttcaaaatcaggtcaaaattacatgcaattttgtaatctgacaatttccacttaatattttattagcattttcctgttatgaaacagtaattttagttatgggtcgttgttttgctatgcggttgggataaaattttatatactttttttggcaattacttattatacataaatgtttgtgtatagttttctttttctgagaattcctggaagttgagttaccaggcccggctttgaatttttttttttattttttttttgagacagagtcctgctctattgtccaggtgctatctcggctcactgcaacctctgtctccctggttcaagcgattctcctgcctcagcctcccgagtagctgggattacaggggcacaccaccacgcccaattaatttttgtatttttagtagagacagggtttcacgatattggccaggctggtctcgaacttctgaccccgtgatccacctgcattggcctcccaaagtgctgggattacaggcgtgagccatggcgcctggccaggctttaaatttaaaacaaatcttctaatagctttatggaggttataatttacatttcttgaaatgtactcactttgagtgtatagtaaactccaattttatcacatttctgtcaccccaaatgtatccttgtgcccatttgctgtaacctccggttcctgccccaactcctaggcagccactcatctattttctgtcccttaagatttgtgttttcgccaggcgctcatgcctgtaatcccagcactttgggaggccgaggttggtggatcacttgaggtcaggagttcgagaccagcctggccaacatggtgaaaccttgtctctactaaaaatacaaaaattagtcggatgtggtggcacacgcctgtaatcccagctactcgggaggctgaggcaggagaatcacttgaacctgggaggcggaggttgcagtgagcagagatcgcgccactgccttccaacctgggcaacagagagagactgtctcaaaacaaacaaagatttgtattttctggacattttatagtactggggtcatagtatagatggacttttgcatttggcttcttttacttaattgtgagattggttcttgttgtagcatgtatcagtagtttgttcatttttattggcgaaagtattctattatatgaataataccatattttatctatccatcagatggatattatagagttcatgttttggctaatttatgaattatggtactgtgaacatttgcctgcaagattttgtgtagacatgtcttcatttctcttgagtagatcacctagaagtggatttttaaataattttggtacttactgtgaaactgctcttcaaaaacataccattgttccttccttccttccttccttccttccttccttctttccttcctcccttcctccctcccttccctacttccctctccctttccctttcccttccccttttcccttccccttcccgcctgcctgcctgcctgccttccttccttccttccttcgtttctttctacatatacacatttttttaaatttcaatggtttttggggtacaagtggtttttggttacatggctgaattttggttacatggtgaagtctgagattttagtacacctgtcacccgagtagtgtaccttgtacccaatatgtagttttttgtccctcaccttccagccttccgccttgtgagtctccaatgtccattataccacactgtatgcccttgcgtacccacagctcagctcccacttctgagaacatatagcagaaacatgccaaagtatactcccactaccagaatgtgattgtgcctgattcttctcaccagtacaaatatttcaaaaaaagttaaatatgtatcagttttttgggcagaagttgatacttctctttatttatttattttttttgagatagggtctcattctatgatgcccaggctggagtgtggtggtgcgatctcggctcactgcagtctctgcctcccaggttcaagtgattcccacgtcagcctcccaggaagctggaattacaggcgagggccaccactgccagctaatttttgtattttttggtagagatggggtttcaccatgttggccagactggtctcaagctcctgacctcaagtgatccacctgccttggccttccaaagtgctgggattacaggcgtgagctaccacacccggctgatatttctttttaaaataacttaccttcttttgaaagtaatacatgtttaatgaacagaatttaaggaaaatataaaaaaacgaaataatctttgtaatcaaactactgaaaagaaaaccaaagttacattttggtgcatattctttttcattttcatcattgtaatttgcatttctttgattacttgtgagacactcctttcatttacttaataggtttatatgacttgcctattcagagattttgcagctttaccattttctgcaaatgatagcaacttctttttgtttgtttgtttgtggagacagagtctcgctctgtcactcaggcaggaatgcagtggtggaatcttggctcattgcaactattgcctcctgggttcaagcgattttcctgcctcagcctcccaagtagctgggattacaggagtgtgccaccatgcccggctaatttttgtatctttagtagagatggggttttgccatgttggccgggctgatcttgaactcctggcctcaagcggtccccctgtctcggcctcccaaagtgctgggattacaggcgtgagccaccgtacccagccagtagttacttcttatattctagaaaaaattctactcatgatcaagtctccatgaggaaagagactttaattgaagatcatggggcttgcagaccaatatgataaaatagttcattgtttctaaaagtattactgagtgttgatggcagatatgaacccttttgtttttgtaggaaaatgttacccgtattctccatttgaattcagtttagatttgttaggaatcgcagcttaagctttgccatctgggagtgtttgggacagttttgcagacaaaattgcaaaagtgcctaaggaatgcagctggcattcagacctgctctgtgctcagtactctgtggacagacactgttcagcacttgttgatcagaaggtttagaaagagaactttcaaagttggtttttaattaaagcatttaatagtgtaaatagaaagggattaaattttatgacagacaaaagaaagtacagcacccagctgggcgtgggggctcacgcctgtaatccagcactatggggggctgaggtgggtggatcacgaggtcaggagttcaagagttcaagaacagcctggccaaggtgatgaaaccctgtctctactaaaactacaaaaattagccgggcgcggtggcaggcgcctgtaatcccagctactcaggaggctgaggcaggagaatcacttgaacctggacggcagaggttgcagtgagccaagattgcaccattgtactccggcctgggccacagagtgacattctgtctcaaaaaaaaaaaaaaaagaaaaaaagaaagtacagcacccagttatgtccgagtgggtgcatgagagtgaccctgagattggagacaacgctgtcacgtgcttgaagaacgccacctgagaaagggggcgagaagtggtgtccgctggtaaccagaggtgttggcttagccatctgcagggaggagggtggtctatcacaggtgagtttcatctactttcttaagcaaattaaccttacttttgtgttaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccaggtatgctgacccagtggcatcttcacattgtcgggaaaatgccctttcctgatgcctttctttaggctttaattgaaaacattttattttctagaaaaaagcttcagctcaggatgtttgagtgtaggtcagtcctttgataggatattatcattttgaggattgaccacaccacctctgtatttaagctctgccacaatcactcagctgtgacactgtaaatctcttaatagtttattacattccatgtgctgacagttgtatttttgtttgtgacacttacgtattatctgttaaaacattttcactttagttgtgttacctttaaagaggattgtattctatcatgcctgttgattttttggtgagcgggctattaaagtcagtgttatttagggttatccactagttcagtgatttgcgagattatcattcacatttattgtggagcttttgaatatcgtgtcaaatggccacatatatcccattcttatctgcttcttaggtgagtgggacacagtgctttaatgaagctataatcttcagaattctagcttgcagagaagattgcagaagtgataagacttgtgctttttaattttgtcttttaaatgttattttaaaaattggctttatatgatactctttttttctgctgagtaacagtgttttacaaaacttggactaaatgacttctaagcttaaatgatcacttgatgctttttttctgaattaggaactcagcttatcaaatatcaaagtcataattcctgaataaataacgtcttttttcatgtaaagactgctttaaaaaacacatggaaggctgggtgcggtggctcacgcctgtaatcctaacactttgggaggcccaggtgggcaggtcgcttgagctcaggggttcaagaccacccagggcaacatggcaaaacccacctctactcaaatacaaaaaattagccaggcgtggtggcgggcccctgtaatcccagctactcgggaggctgagggatgagaatcacttgagccccggaggcagaggttgcagtgagccaagattgtgccattgcactcccagcttgggctacagagtgagactctgtctcaaaaaaagacacacacacaaacaaaaaaaacatggagacatttttttggccaccttaatatttcccctcagataatttcctttgtttaaactcagaactggcattttctctcttggagaagattcaggacaaatactcctttaagataagtagaagcagtgaaagaggatttgattatcaggaatttgataagcttagaataaattgttgcttcttaatgtcatttcagaagatgaatatttattaatagatgccaactgagatatcattaaaattgattactaactactacttggaaaagtctcccagttccaaacttcagcaggcctcttgacaattcagctgtggtcaattgggtcttgcgtgatagatacaatgaccaattgtgcagcagagtgtgctgcttagctgcctattctgttagcattcatgtgttaacttaaaatcataatctccttagttttgttgagtgtctccgtggacaagacactgtgagggatacaaaatcagattggctttattcaaaccactggggtattataattcatttataatttattttattttttgccttttttccatgtgttctaaaggaattagagtttgtatataactataatgggggatagaaattgacatgtgccatgaagggaatgcaaaaaagtgccgtgggagatgagaagtggagaaaggaatttcttttttcttggaagcaggaataacttcatgaagcatgtatttcaacttaaacagatagtaggcaacgctgtaaggggagtatggctgcagcaaaagtgttcggggcagactgggaggaagggagggaataaattcagccattgttatggaataatgatcaaaatttattttcagcccgtttcacttaaaagttgagactgcttaactttttttaatctttaatcttaaacttttaaatgccatttgatctttaaaaatatatgttttaatagtgtattttaagtctctatatttttgttattagaatatatagaggctataacctactaccaagcataacagacgtcactatggaaaataacctttcaagagttattgcagcagtttctcatgaactaatcacatcaaccaccagagcactcacagtaagtctctttcttgatcggtcttactgacattgtaatagtttttggtagcttgtatggccagttagttgtatggtcatcttacggtgaggtgcttgtcttacagctcttacttatccatgaggcttgctaagaaattgtgcttctgtgaaaagaatctcagcttactccaggaatgtaaatgactatgttttttctgattattaaagtaatacacgcccaaaataaaaaaattcagccaatttaggaagacacaacaattaaaataagccaggcatggtggctcatgcctgtaatcccagcactttgggaggccaaggttgggggctcacttgaggtcaggagtcggataccagcctggccaacgtggtgaaaccccatctctactaaaaatacaaaaattagctgggcgtggtggcgggcgcctgtaatcccagctactcaggaggctgaggcaggagaatcgcttgaacctgggaggtagaggttgcagtgagctgaggtcaagccactgcactccagcctgtgcaatagagcgagactctgtctcaaaaaaaaaaaaaaaaaaagaaaagaaaaaagtaaactactgtcacctgcattggtaatgtatcagaagtttaaaatgtctagattataattaactcagtgacctggtaatatatactaagggaaaaatatttataatttacatttttacatttttatttttttaattttattattttttttttgagacagagttttgctcttgttgcccaggctggagtgcaatggcatgatctcagctcaccacaacctccacctcccgggttcaagcaattctcctgcctcagcctcctgagtagctgggattacaggcatgcaccaccatgcccggctaattttgtatttttagtagagacagggtttctccatgttggtcaggctggtctcaaactcccaacctcaggtgatccgccctcctcgaccccccaaagtgctgggattacaggtgtgagccaccatgcctggccttacatttttataataagaatttatgttgctgacattagaaaagaaccataatatccaagaatccaagaataattaaattatgtacatatgctagtatatagtgtgatgctttggagaatttttaacaatatggagatgtataatctggattgtaatattgagtgaaaaaaggcagaatacaaacctggtgggggtatagtcggatttcagttaagaaaaataatatttacatatatacatttctcacactggcagataatcaccaagataaattttgggattgtggatgatttttttcttctttatatttttcagatattctcaaattttctaaaatgagcaagtataacttttgttatcagaaaaaaataatatacaaaagtaatgttaatttgctggtgaccaggttaaacctttttatttttattttttgagatggaatctcactctgttgcccaggctagagcacagtggcatgatcttggctcactgcagcctccgcttcctgggttcaaatgattctctggccccagcctcctgagtggctggaattacaggcgtgtggcaccacacctggctaatttttgtatttttagtagaggtagggtttcaccaggttggtcaggctggtctcgaactcctgacctcgtgatccacccacctcggcctcccaaagtgctgggattacaggcgtgagctactgcgcccagccagacctttttattttatttgacaaaagaaatacttccatgttatagaagactaaatattgtttgggctgtctgcagtatggtcttcccttgatttgttcaaaatatcgtaaactttgcttatttatttttattgtggccgactgtgtcgggcactgttgtaggcttgggatggaaaaacaggattcctgcccttagggtttctgcaggctggtcagggagacgatgtggtaagctggagctcagctcctaaggatgtgcaggggcagttgagaggcggaagggtgggagatcattccagggtgtgggcagcacaggaacctctcttcattgggatataattgccattctgataacacgtgtttgaggtgtctaaagtaggaagttgtaccatggtgggacagatatcctgtggttatcatacacagatctcagttttcttctcattgtttgtactttttataaagggtaacaggagatataattcaataaacctttgtggtgtttgggtgtgattttattgtttctttcttctcagtttggatgctgtgaagctttgtgtcttctttccactgccttcccagtttgcatttggagtttaggttggcactgtgggtatgtattttcctcagtatatattaatagttgtctacaacagtatgacataaacatagttattaggatgccctttttctttctttttaagtcttttatcaatttggctttttggaaaaatatctgatggaatacttgtttctgctatattagctgtgtgagactagtgacaggagctgtgggaaatgaatgccaaatgttcttaggcattgatgggaatttcagggtgtggtcttcaagttcatttaagggaattttcatatgctggcaaaaggcttttctcattagcttgactctttccaaaattatttgctgtgaattagaagtttaggaaccttttttcacttaattgtgacctagcatacgaaatggtgatgatttaggaactactgttcttgtattaacagcttttatttaaaaatgattttcctccagtagatggccctactagcatctgggaaataatttcaagtcttctccagcattcaggaataggctttcattttgtgtatcaattactgagaatgattttggtgactcacatcacatttgagaagtaaacctgcagatttcttgtgtgtgtcagcaaatgaccaactgatatttgcttgaagtggattacattatctgctctagaatgattgctttcccaccttcctcacatacagactgagcagctacggtttctaatcataggtctggcactagacttcacttctgggcaactttggcattggagtaaaatgtattaatttaaagaaagttaaaaatccgttcaagtaaacatacagttctaatactttttacaatttaaaatatagatttaaatgataaaataaaaaagaaaatatgggtagacaccataatcctcgtttctgcatctgttcacaaggggttgatatttatgagttctattctccatatccattctatgttctcttaatgctcagtcagcacctcaggtggttggagttcaatgcttggtagtttgacttacactgtcttttctaggggattgagccctgggtagtcctgcttatttgaggttgcaatttgtctttcaataacttttactacaagatatggcgtgttaaaggataccattggggaaccaacataataatatcaggaaaactaaccacgtcagacctgccccattgtgtatcaagtacactatttttccatagtaataaagagttcaccccagccaattctcttttattttgtgcctgtttactcaatggcattaacatgcccaaatgtctgggtagctgtctcatctccagttcagcagaaccattgtcatatgccctagtaaaagcattccttcattggacacttaggccccaatactttcattcagatctactacctgatttcatttctcaaatgatttttatggagctctgatttataggaaagatgttagttgattaaaaataaaacaatttctgagctggtataaaatgtattgtgacatgccttcctcttggaattgcaagagaaaggaagactgttgtttgcttaaaaattgtctataatttgactttgcaaatgtctgcttccagagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcaggtactggtactgagttgaaacagggactccaggacttggattttgatttccttagggggaatgggggtggtgagcatatgaggggaaaatactataaggtcattgccagtgatggcttgtccctttagtcaaatttcagatgttacctatatgcataaacacatgcagttggcagctgttctgtgctgagtattttaaagtagcctcttcccaatatagcccctcagttaactacaagtaaactcattttgaatttcattttaatgggcaccatatgccagtactccctcgggcactgggatgttaagaaagtataatgtatggacttcattctcaagttagttttagattagagggggatacacgtaaacaaaagtgcagtggtcacacagagtggccctaatcactctccttgggcagatttatgggctggtaggaaagagcacaacacggagagggtgtagcaccttggcgatgataatggaggatgtggccagcaaggaagacggagtccattgaaattgattttgggagaagttgccaatctccatgaaagaattggggcctgtgctatttgcttcagggggctataggagagtttcgtgaaagggactaaaagatgagtattttaataagatcattcatccaacttgaacatgggctggaggagaaggtagggagactcaggagattaatgttgatgctaaggcaagataatggctttgggactgtagggaagacactgattgtaagagaatgaaggaggcagaattgccaggcctggttcaccaactgaacttcggttgtgaagacaaagaaacctgggatgacttcacatcctgggcaggtgtgtggtggtgacagtcatggaaattgggaacacagatttgtgcgggaaacatcagtttcagtttgagtttggcttatcagttgaatatcaggcacagatgtctggccaactctcaacatagggtcttaaatgacttcagttccccaagcaatttgtccttcccatgctattggggtggagaggtaatgtctgtgcccatatcacagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggtaatgtcccacttgggtgctggattcatacagccttaatgactatgggtttccagactacctttgtttagtaatctgtcccttctttattctctttttgctttaaatgaacaaaattgctcagattgtgacactaaatttaacatcaaaatgtgaccatgtggatgggtgcagtggctcgtgcctgttattccagcactttgggagactgaggcaagtggatcacttgaggccaagagttcgagaccagcctgggcaacatcacgaaaccccctctctactaaaaatacaaaaaattagatgggttgggccgggcgtggtggctcaagcctgtaatcccagcactttgggaggccgaggtgggcggatcacgaggtcaagagatcaagaccatcctggctaacacagtgaaaccccgtctctactaaaaatacaaaaaaattatctgagcatggtggcgggcgcctgtagtcccagctgctcgggaggctgaggcaggagaatggcgtgaatccgggaggcggagcttgcagtgagccgagatcgtgccactgcactccagcctgggtgacagagcgagactccgtctcaaaaaaaaaattagatgggcatggtggtgcgtgcctgtaatcccagctacttgggaggctgaggcaagagagttgcttgaacctgggaggcggagtttgcagtaagccttgattgtgccgctgcactccagcctgggtgacagagtcagactctttccaaaagaagaaaaaaatgtgaccatgtgttttatagctcttttagtatcatcagtcactgttatccctaagagggaaatacctagctttagttttaggtttccagcattagccaagaaagctcagaattgatgttcctggccaagtacctcattgctgtctccttaaatcttggttaatggctactgtcctggctagcatagttatggagcatttccatggttgtagaatgttctgccaatctcagggacagttttgcttttctgtgaagcaataaaatcaacttcaaaacaaatgttaactatttgtacaatggatttaagatagaccagttcacatacttttttttttttttttttttgagatggagtttcattcttgttgcctgggctggagtgcaatggtgtgatctcagctcactgcaacttctgcctcctgggttcaaacgattcttctgcctcagcctctcgaggcagattacagctgggattacaggcatgcaccaccacacccagctaatttttttgtagttttagtagagacggggtttcaccatgttggtcaggttggtctcaaactcctgacctgaagtgatctatccgcttcggcctcccaaagtgttgggattacgggcatgagccaccacgcccagcctaagatagaccagttcacttactgtttatatctgattactctctctttgccttgtcttctacctttaaaaatctccctactaacttcccattctcctttagctgccatcagtcttctcccttctctgcaaacatctctggagagtcccagcctcagcccacagagcttcccactgctctgaggtggaccttgtttgcaaggcttctttggctctcttggcctggaccctgtctactacttcagccatccttccttaacccctgctggtggtttctgttgccacactccatagcagcgtttcccgcccagatcatgtctttacatctctgggcactgctctggtcctgcctgcctttccctctttgtatcctgcaggctgctacccccatcttgagtgtcctcttcagttggctttcagagggcctcctgggtgttcccttacccacttgccactccccagtcactgggttcagtccttcctgcccaccagcacatgctttctaggctctgtcctaggccgtcttctctctttgtagtctctgggccagtgctgttctagagagtggcagaattttctataaccatggcagtgctccatagctatgccaggcaagacagtagccactaaacacatatagctgttgagcccttgaaatgcagctagtgtgactgaagaactgaaccccgattcggtttaattttcattaaatttaaatttaaataaccttatgtgggtagtggctccagtattgggcagggcagcctgagagtcggggctgttctcctgtcttcagtgtctagatgagggacctcagaggacctgtctctggagctgcagttcaatgtagccagctgccccgtgacacttacatatagctgatttgtggatatgtcagacacggtgtgatgagctcagctttctgtcctcctccccacatctgcccctgccccatttaccccactttgtgtcttatcaagctagaaacaggtcaccacaagtcttcatttccactcaccaagtcttttgtttcccctactaaatattttgcgagaagaaagtgtgtacctttgtattcacatacatgtacatgcacatatacatgcacatatgcaggggtccccaacctctgttaaaaaccggactgcaggccgtgcgtggtggctcacgcctgtaattccagaactttgggaggccgagaccagtgcatcacaaggtcaggagatcgagaccattccggctcacacggtgaaaccccgtctctactaaaaatacaaaaaaaaattagccgggtgtggtggcgggcgcccatagtcccagctacctgggaggctgatgcaggagaacggcgtgaacctgggaggcggagcttgcagtgagccgagattgtgccattgcactccagcctgggcgacagagcgagactctgtctcaaaaacaaaacaaaacaaaaaaaaaaaaaaccaggctgcacaggaagaagtgagcaagcattaccatctgagctctatctcctctcaggccagtggtggcattagattctcataggagcgtgtatgagttcgttctcacacttctgtaaagacatacctgagacatataaagaaaagaggtttaattggctcacagttctgcaggctgtacaggcttctgtttctgggaaggcctcaggaaacttgcagtcatggcagaaggtgaaggggaagtaggcacatcttcacatggcccacaggaaaaagagagaaggagagagagagagagacagagagagagagagaaaaagaaagattgagagggagagaggagggagaaaggagagtgcctgtagggggagttgctacacaaaggagcaccagggggatggtgctcaaccattagaaactacccccatgatccaatcacctcccaccaggccccacctccgacactggagattacaattcagcatgagatttgggtggggacacagagccaaaccatatcagagcatgaaccctattgtgaactgcacatttgagggatctaggttgcatgctccttatgagaatctaatgcctgatgatgatttgaggtggaacagtttcatcccgaaaccatcccccgccaaccctggtttgtggaaaaattgtcttccacagaaccggtccctggtgccaaaaagtttggggacctctgcacatatgcatgcacctgtacatggacacataatacatgtacatatgcatactttatattctctgccacttctggtccagactgatatactatctcatttggattactgcactagccttttgttttggaaacagcattttttaaaaaatttaatttaatttttttgagatagggtgtcattctgttgcccagcttggagtgcagtgtcatgatcatagctcactgcggcctcgatctcccaggctcaagtgatccttctgcctcagccttctcagtagttgggactacaggcatacccaccatgcccagctaattttttgatttttttttttttttgagacagagtctcagcctgtcgcccaggctggagtgggttggcgcgatctcagctcactgcaacttctgcctcccaggttcaagtgattctcctgcctcagcctcccgagtagttgggattacaggcgcctgccaccacacccagctaactttttgtatttttagtagagacggggtttcaccatgttggccaggctggtctcgaacttgtgacctcgtgattagcccgcctcggcctcccaaagtgctgggattacaggcgtgagctaccgctcccagccaggaaacagcattcttgagataattcatataattcacccatttaaagtatataattcattctctttagtatgcccacagagttgtacagccatcaccagaatcagttttagaacccataaaggaactctgtactctttacccaaaacctccatgcctccagctgcaggcagccactaacctgccttctgtctctgtgactctacgtcttctggacattactgtggatgggctcatacagtcagtgagcttgtgactggtgccttctaccaagcagggttttcagtgtagcagcctctctgtttttcttttttttttaaattgtgacggaacttctgcctcccgggttcaagcgattctcctgcctcagcctcccgagtggctgggactacaggcccatgtcaccatgcctggctaattttttttttttttttttttagtagagatgggtttcaacatgttagccagggtggtctcgatctcctgacttcatgatccgcctgcctcggcctcccaaagtgctgggattacaggcgtgagccaccatgcccggctaacctttcatttactgtctgcatttcttccctgatgccttccagtccatgcacccgattgtagccattcatcctattatggtttaaggtgactgtcttagtcagcatgggttgccataacaaaataccatagcctgggtggcttcaacaacagaatttacttctcacacttctggaggttgggaagtccaagatccaggactttcgccttgccctcatgtggtgagggggtgaggaagctctgtggggcctcttatatatggatgctaatctcattcatgaggggtctgccctcatgacccagtcacctcccaaaggccccacctcctaataccatcaccctggtaattaagtttcagtgtataaatttgggggactatagacattgaaaccataacaagcacttttctaagatcagggagtgagtaagtagcagagctaggacctcaattccacatgtcagtcatcttgccttcactctgctccatgatggctgcctcctagagcattgggagtctcgatgttctatatgctctcatgtgttgtgtattggagatagttgaggctttatgaatacatctggatttgttgacttctagctttgctggtaaccagctgtgaccttgaataagttacttcatctctgagcctgtttcctcttttagaaacaggagtttaaaatgctgctttgggttgggcacggtggctcatgcctgtaattccagcactttgggaggctgagatgggaggatcactggagcttggagttcgagaccagcctgggcatcatagtgtgagatcctgtctcctcaagaaattaaaaaattagctgggtgatgtggcgtgtgcctgtggtcccatctactctggaggctgaggtgggaggattgcttgagcccaggaggttgaggctacaatgaaatatgattgcaccccatcctgggtgacgagtgagaccctgtctcaaaaaagaaaaaaaaaatgctgctttgtacccctttcatgtcatggcgtcatggccaacatagaatgccctggttgtttgctgttggagggcatgggcctgggggctccctgagggctccttccatcttcaactcattctctgtgcacctgttaggaagttgtgggccagtccctaccatgtatcattgtgtgggtaaaagtaaataaaatgtgtacagtgtctgaactgtacatatcagggtccaagaacaaaatgagtgacatgggttagctctttttaataaatggtaaaaccaaatattctaattttcagttttgttatacttccatcacatgtttttgtttttttgttttttgtttttgtttttctattttaggcagccttgccttctctaacaaaccccccttctctaagtcccatccgacgaaaggggaaggagaaagaaccaggagaacaagcatctgtaccgttgagtcccaagaaaggcagtgaggccagtgcaggtaggaaacagcgtggggaagggagggacatgagtgcagcatctgtcatgtagaaacataggatttaagtaacttggtgttttagagaaataaatataatacacatcagtaaagtgagagaaagtttctccaggtgcggttcaagatattagaaactaatgactgatgtacacagaccaccttttggtctgaagcatttctaagtgccactggctgacatgcagcccctacagcctccaggcttccagccctagcatggagcatcactctcctatgcttccctggttgcaggtgatggctggagaggcctcctgattttcagtaagggaagtggtgtagatgcttaggaatagatgtagtgagtgaaaaaactgattctgatatgtcaaaaattctgattggaaatggaatatttacatttggaagagctaaaggcgagagaaagtggggataaagtcatctgagttggaggagcttaaaccattcacaagtttggaggacctttttttacccatgaaaaggtcagaacagaaggggctaggatttaggtgtgactgcagtttattgaattcccatccatactgctctcggtgggcagtggcaggggcaggagaggagcctggcaaagcatgaagtgactgctgctgcctctgctatctgggacgcctggccacctgtctgtacagtctccctccagacccattctcacgctgtctcttggcacccaggggccagtgatggttctcccatttgttttgtgtatatagcatttatatcaaggctatttatttatttatttattttatttatttatttttttgagacagagtctcactctgtcacccaggctggagtgcagtggtgcaatctcggctcagtgcaagctctgcctcctgggttcaagcaattctcctgcctcagcctcctgagtagctgggactacaggtgtgcaccaccacacctggctaattttttgtattttttattagtggagacggggtttcaccttgttggccaggatggtcttgatctcctgacctcgtgatccgtccacctcagcctctcaaagtgctgggattacaggcatgagtcactgtacccggcctatttatttatttttaattgacaaaattgtatatatctgtaatatacaacatgatgtttgaaatatgtgtacattggccaggcgtggtggctcacacctgtaatcccagcactttgggaggctgaggtgggcggatcacgaggtcgggagttcaagaccaaactggccagcatggtgaaatcctgtctctactaaaaataccacaaaaaaaaaaaaaaaaaaaaaaagccgggcatggtggctcgcgccagtcgtcccagctacttgggaggctgaggcaggagaattgcttgaatctggcaggtggaggttgcagtgagctgagttcatgccactgcactctagcctgggcgatagagcgagactccgtctcaaaaaaaaaaaaaaaagaagaaatacatatgcattgtggaatggctaattaacctgtgcatcacctcacgtatcattgttttgtggtgagaacacttaaaatctactctttcagtgattttcttgcatatggtacattgctattaactgcagtcaccatgctatacagtagatctcttgaactcattcctcctgtctataaatgaaattttgtatccttgaccaacacattcaaggttttttttgagatggagtcttcttcacccaggctggagtaccatggcacgatctcatctcactgcaacctccgcctcccaggttcaagcaattctcctgcctcagcctcctgagtagctgggattacaggcacatgctactgcacctggctaatttttgtatttttagtagaagtggagtttcaccatgttggccaggctggtctcgaactcctgacctcaagtgatccgcctgccttggcctgccaaagtgctgggattacaggtgtgagccactgcacccggcctcaagcgttttaaaagatgctcttttctaaggattgactgtagtacaggaggaagattgacctgttgaaaagcctcagcctttacaagtgtaaaattatcagtatattactatcatctttctgatgaattaaataaactaaggactccaagtcaaaagtcttcaaactgaagtagaatagttgtatatagtgcttggcactttaatatttagtatcggtttaatgataatgtttgtgcctttgccgtctttaaaacatttttacatcatccctgtttgattacttggtgtgctcatgaagttgttggccactaaggaatcttaggctcagagaggttctggaattggccagtggtccttgaatcagctgctcctatgattctctaactgatttctcacaaagcaaacaagcaatcataacaaaacaactgtgcacactgctcttcttattttgttatttaaaaagtacttaggctctacttatgtttgttagtcaatttctcattacttctagttaatcaaaaggtcagaggaaatacttgaatattttcatactagaatactttaaaaaatcatgatttccagtaatctctttaaaacttggcaagttattttgatctaaaagtttatcttttgtgtgcatatttttaaagcttctagacaatctgatacctcaggtcctgttacaacaagtaaatcctcatcactggggagtttctatcatcttccttcatacctcaaactgcatgatgtcctgaaagctacacacgctaactacaaggtatgggcctctgcatcttttaaaaatatatatgcacacatacttacgtctaatggatagttgatgtttttcttatgatttgtaggatgtataagccctttgagatatgagttacatttagttttttcaagtttgtttgtctttcagctttgtttatgatagcttctatcatacaggtgttttggattttcatattgtttgtactcacagctaagattgattacagtgacagagctaggatgtgcagccaggttatagggggaagtggccctggtggagtctggagggatccgtgtacaggcttccttccctcccgtgaggctcacacaaaaatacagcaacatgctggtcctgcaggtaccctctgcctaacatgagccacaattccagactcacagaagaaaagcaggtgttcggcataaaccatgtgtttcaaatagtctgggcatggtgagccacttgttatcagctagggaaagtttatgtcagcgtaagaaactgttcaccagatacccccaagagccagcctttctgtctagggatgttttagttttttagttcatttttttttttaactttaaaattttctgttcatctgcaatttgttagatatgaagtatgtgtctaatttaatttttgtttttggttgtccccaataatgtttacagaagaatttttctgcactaattggcttgagttacttacattctcatagttctctagtttcagtagtttcatttattattttgttatatcaatctatctgtctgctcatctattagaagcatccttgttttttttttttcttttttagacagagtcttgctctgtccccaggttggagtgcagtggtgcaaccatgcctccctgcagtctcagggctcaagtgatcctcccacctcagctcctgagtacctgggactaccggcatgtgccaccacacccagctaatttttacattttttgtagagacagggtctccctaagttgcctgggctggtctcaagctcctggcttaagtaatcctccctccttggcctcccaaagtgctgggattacaggtgtgagcaactgcacccggctacaagtatacttcttaattattgtagcttaatggtatttatgaggggatcagttcccctgttgttctttagaattttctggatattcttctttattgattttgggatgtgaacaatagaatcaacttctacttgtagattgatttagggagaacttatacctcagatgttaagtcaccctgtccagaatgtgggatgctttcctatttgttcagaactttttaaattacctcagaagcacatgaaatttaaaggattttaaaaaaaacttaaagattatttcacatagctcttgcacatttcttgataaatgaatcctcaggtattcctctgtttttgttactaatagttacttcttatgggttttttttcccctgaaaatcatttatcaaacgtatgtggcttattttctgaaggatgtttgataattttggaagatatgaaagtcttcatattttacaaggtttgaggtctctttaagctgcatggttctcatgtcagctcccaaagcagaagacggcatgttgaaaaatgccgtagagaagatacttcttttccacctgttttcaactcatatcatcttgaatttcagggcacctttccatgctcctagtgcttgctatctgtttattattttccttcctgaataccctgaactccagcatgttctgctgtaattctggcctccctggcatcttggactcctgtttcctttgctctgtcatccccgcggtcagctcctgctgcgcagcttctcagctgaagtgcgtttggagtgcctggcgtgtcttgctggatctttgagtattgcctctggtttccttggttccttctgctgagttgctcagcgtctccactccccatttcttgtgtggcccttcctgcactcctctgattccttttgtcttccctggtttcttgctttggtttcgagtctccacagaacttttgcagctcttctgaagacctggaagctttttcatcttaattctcatctcatgacctcttttcccttctttgagagctagaacttcccatggtgaacttctctttccagaattccatgccttcttttccctcccacttacctgttgtccaggagaggtcagattgctgtgcatattggaggagaaccctttcttccctgggctcttcatctcacatgacatcaccacatcacctcgttccttggaccctcagtggtgtcactgctggatttttctttcctttggctggccttagggcacacccaggttgactagcgtagtcatggtatttagatccactcacattttcagtttctgtgtctgtctcttgcctgcttctgacttcgcccagagaaagcttctctttcacaagggttcttagatttatgttcactgagcaccttcttttctgaggcagtgttttaccaatatttattttcctagtcagtctcgccttacctttcttgttatgcatgtctttggtcctgacccattctctgagtctgtaaaatagaattgctgtataatttaattacatgaaatcctttagaatcttaacacatcttacacctgatttaatattttattgtatccaaattgaaccaaccctatgtgaatttgacagtgatttctcccagggatcctagtgtataaggaataggacttagtattttctattttttgatataccacataccagatactgattatgatggacatttaacccttttttctcattatgaaagaaagttaggaattatttcttccagtagcgccagtgtaacctgaaagcctttgaaagagtagtttttgtatagctatctgaaaggaatttctttccaaaatatttttccagtgctgacaacaaacacgcagacacaccctgcaaggtgagtgtacggcgccgcacagtggaggcatctgctgcagccgtcgatgtttgtgtctttggttgtacattatgagatcgtgacagggccagtaaccgtgtgttctctccttcaccttcccaaggtcacgctggatcttcagaacagcacggaaaagtttggagggtttctccgctcagccttggatgttctttctcagatactagagctggccacactgcaggacattgggaaggtttgtgtcttgttttttctccttgggttgtggctggcacacttgatgtgcgtcttctgggctgagttcatctaggatggagcctggttctccagggtgcctccgggagactcctccctgccccacgtgcttgcgtcacaggacccaagtctgactctgccttagccatgaagtttagggggaagtttctatttgtattctatttttgtctgttatcatgtattagcttagacccagtttagtttggaaaatcagtgggtttcaaaatgtgtttgtagagtcctttatttcttaacttgaccttttcaagtggaaaggggcaaaacagacgggtaagggggcggggcgggaggtgtgacttgctcttttgtgcctgaggaagtaacagagctggggttgacagtcatattctctgacacagatagtctctgacttatctcacagaaagtcagcggcagagcctgagttaaaagtctcgtagattttctttttcttttttttggtggctaatttcagttttatttatatttgtttatttatttattatactttaagttctgggttacatgtgcagaatgtgcagttttgttacataggtatacacgtgccatgatggtttgctgcacccatcaacccatcacctacattaggtatttctcctaatgttatccctcccccagtcccctcactccccatgggccccggtgtgtgatgttctcctccctgtgcccatgtgttctcattgttcaatttccacttgtgagtgagaacatgcggtgtttggttttctgatcttgtgatagtttgctgagaatgatggtttccagcatcatccatgtgcctgcaaaggacatgaactcatccttttttatggctgtatagtattccatggtgtatatgtgccacattttcttaatccagtctatcattgatggacattcgggttggttccaagtctttgctattgtgactagtgccacaataaacatacatgtgcatgtgtctttatcgtagaatgatttataatcctttgggtatatgcccagtaatgggattgctgggtcaaatggtatttctagttctagacctttgaggaatcgccagactgtcttccacaatagttgaactaatttacactcccaccaacagtgtaaaagtgttcctatttttccacaacctctccagcatctgttgtttcgtgactttttaacgatcgccatcctaactggcgtgagatggtatctcattgtgattttgatctgcatttctctaatgaccagtggtgatgagcattttttcgtatgtctgttggctgcataaatgtcttcttttgcgaagtgtctgttcatatcctttgtccattttttgatggggttgtttgcttttttttcgtaaatttgtttaagttctttgtagattctggatgttaatcttttgtcagatgggtagattgcaaaaattttatcccattctgtaggttgcctgttcactctgatgatagtttcttttgctatgcagaagctctttagtttaattagatcccgtttgtcaattttggcttttgttgccattgcttttggtgttttagacatgaagtctttgcctatgcctatgtcctgaatgttatggcccaggttttcttctaggatttttatggtcctaggtcttatgtttaagtctttgatccatcttgagttgatttttgtgtaaggtataaggaaggggtccagtttcagttttctgcatgtggctagccagttttcccaacaccatttattaaatagggaatcttttccccattgcttatgtgtgtcaggtttgtcaaagatcagatgattgtagatgtgtggtggtatttctgaggcctctgttctgttccattggtctatatatctgttttggtaccagtaccatgcagttttggttactgtagtgttgtagtatagtttgaagtcaggtagtgtgatgcctccagctttgttcttctagcccaggattgtcttggctatgcaggctcttttttggttccatatgaagtttaaaatagttttttccaattctgtgaagaaagtcagtgatagcttgatggggggatagcattgaatctataaattactttgggcagcaaggccattttcacgatattgattcgtcctatccatgaacatggaatgtttttctatttgtttgtgtcctctcttatttccttgagcagtggtttgtagttctccttgaagaggtccttcacatcccttgtaagttgtcttcctaggtgtttcattcccttagtagcatttgtgaatgggagttcactcatgatttggctctctgtttgtctgttattggtgtataggaatgcttgtgatttttgcacattgattttgtatcctgagactttgctgaagttgctaatcagcttaaggagattttgagctgaaccaatagggttttctaaatatacaatcatgtcatctgcaaacagggacagttttacttcctctcttcctatttgaataccctttattgctttctcttgcctgattgcgctggccagaacttccaatactatgttgaataggagtggtgagagagggcatccttgtcttgtgccggttttcgaagggaatgcttccagtttttgcccattcagtatgatattagctgtgggtttgtcataaatagctcttactatgttgagatacgttccatcgatacctagtttattgagagtttttagcatgaaaggctgttgaattttgtcaaaggccttttctgcatctgttgagataatcatatggtttttgttgttggttctgtttatgtgatggattacgtttattgatttgcgtatgttgaaccagccttgcattccagggatgaagctgacttgattgtggtggataagctttttgatgtgctgctggattcagtttgccagtattttattgaggattttcacatcgatgttcatcagggatattggcctaaaattctctttttttgttgtgtctctgccaggctttggtatcaggatgatgctggcctcataaaatgagttagggaggattctctctttttctattgattggaatagtttcagaaggaatggtaccatctcctctttgtacctctggtagaattcggctgtgaatccatcctggactttttttggttagtaggctattaactattgcctcaagtttagaacctgttatcagtctattcagagattcagcttttttctggtttagtcttgggagggtgtatgtgtccaggaatttatccatttcttctagattttctagtttatttgggtagagatgtttatagtattctctgatggtagtttgtatttctgtgggatcggtggtgatatcccctttatcgtttttattgagtctatttgattcttctctcttttcttctttattagtcttgctagcggtctacctattttattgatcttttcaaaaaaccagcacctggattcattgattttttttggagggttttttttcgtgtctctatctccttcagttctgctctgatcttagttattttttgtcttctgctagcttttgaatttgtttgctcttgcttttctagttcttttaattgtgatgttagggtgttaattttagatcttttctgctttctcttgtgggcatttagtgctataaatttccctctacacactgctttaaatgtgtcccagagattctggtatgttgtgtcttcgttctcattggtttccaagaaaatttttatttctgccttcatttcgttatttacccagtagtcattcaagagcaggttgttcagtttccatgtagttgtgtggttttgagtgagattctcaatcctgagttctaatttgattgcactgtggtctgacagacagtttgttgtgatttctgttcttttacatttgctgaggagtgttttacttccaactatgtggtcagttttagaataagtgcaatgtggtgctgagaagaatgtatgttctgttgatttggggtgcagagttctgtagatgtctattaggtccgcttggtccagtgctgagttcaagtcctggatatccttgttaattttctggctcattgatctgcctaatattgacagtggggtgttaaagtctcccactattaccgggtgggagtctctttgtaggtctctaagaacttgcttcatgaatctgggtgctcctgtattgggggcgtgtatatttaggatagttagctcttcttgttgaattgatccctttaccattatgtaatggccttctttgtctcctttgaactttgttgatttaaagtctgttttatcagagactaggattgcaatccctgctttttttttgctttccatttgcttgttagatcttcctccatccctttattttgagccaatgagtgtctttgcatgtgagatgggtctcctgaatacagcacaccaatgggtcttgactctttatccaatttgccagtctgtgtcttttaattggggcatttagcccatttacatttaaggttaatattgctatgtgtgaatttgatcctgtcattatgatcctagttggttattttgcccgttaactgatgcagtttcttcatagcgtcagtagtctttacaatttggcatgtttttgcagtggctggtactggttgttcctttccatgtttagtgcttccttcaggagctcttgtaaggcaggcctggtggtgacaaaatctctgcatttgcttgtctgtaaaggattttatttctcgttcacttatgaagcttagtttggctggatatgaaattctgggttgaaaatactttttttaaagaatgttgaatattggctcccactcttttctggcttgtaggatttctgcagagagatctgctgttagtctgatgggcttccctttgtgggtaacccgacctttctctctggctgccctttccttcatttcaatcttggtggatctgatgattatgtgtcttggggttgctcttctcgaggagtatctttgtggtgttctctgtatttcctgaatttgaatgttggtctgccttgctaggttggggaagttctcctggataatatcctgaagagtgttttctaacttggttctattctccccatcactttcaggtacaccaatcaaacgtagatttggtcttttcacatagtcccatatttcttggaggcttggttcatttcttttcactcttttttctctaatcttgtcttctcgctttatttcattaatttgatcttcaatcactgatatcctttcttctgcttgattgaatcggctgtcgaagcttgtgtatacttcacaaaattctcgttctgtggtttttagctccatcaggtcatttaagctcttctctacactggttattctagccattagtctaacatttttttcaaggtttttagcttccttgtgatgggttagaacatgctcctttagctcggagaagtttgttattaccgaccttctgaagcctacttctgtcaattcatcaaactcattctccatccagttttgttcccttgctggtgaggagttgtgatcctttggaggagaagaggtgttctggtttttggaattttcagcctttctgctatggtttctccccatcattgtggttttatctacctttggtctttgatgttggtgacctacggatggggttttggtgtgggtgtcctttttgttgatgttgatgctattcctttctgtttgttagttttccttctaacagacaggcccctcagctgcaggtctgttggagtttgctggaggtccactccaggccctgtttgcctgggcatcaccagcagaggctgcagaacagcaaatattgctgcctgatccttcctctggaaacatcgtcccagagcacgaaggtgtctgcctgtatgaggtgtttgttggcccctactgggaggtgtctcccagtcaggctacatgggggtcagggacccacttgaggcagtctgttcattatcggagcttgaatgccgtaccgggagaaccactgctctcttcagagctgtcaggcacgtatgtttaaatctggagaagctgtctgctgccttttgttcagatgtgcccttcccccagaggtggaatctagagaggcagtaggccttgctgagctgcagtgggctctgcccagttcgagcttccctgctgctttgtttacactgtgagcatagaaccacctactctagcctcagcagtggtggacacccctcccccagccaagctcctgcatcccaggtcgatttcagagtgctgcgctagcagtgagcaaggccccatgggcgtgggacccgctgagccaggcacaggagagaatctcctggtctgctggttgtgaagactgtgggaaaagtgcagtatttgggcaggagtgtactgctccttcaggtacagtcactcatggcttcctttggcttggaaagggaagtcccccgaccccttgtgcttcccaggtgaggcaacaccccgccctgcttcggcttgccctccgtgggctgcacccactgtccagcaagtcccagtgagatgaactaggtacctcagttggaaatgcagaaatcacctgtcttctgtgtcgatctcactgggagctgtagactggagctgttcctattcggccattttggaagcatcccttgttttttgaggtggagtcttgctctgtcgcccaggctgacgtgcatcggcacaatctcggcccactgcaacctttgcctcctggtttcaagcgattctcctacctcagcctccggagtagctgggattacaggcacctgccaccatgcctggctaattttttgtatttttagtggagatggggtttcaccacattggccaggctagtctcgaactcctgaccttgtgatccacccacctcagcctcctagagtgctgggatcacaggtgtcagccaccacgcccagccatattttcagatctccctctctttgccctaaaccactgtgcttaataagtagtttttagtggccagcagtctccatgtataacacattttagcaaaatggaaaatactatatgttttaaatttgaacgtgagattatactgaaataaaaatcatctaactgggattctttaaatagtaagattttcttttttgtatgtgggtttttttttaaccttattattatgactgtcatatatagaaatggctgtttttcagttacagtcagtgaatgtatcaaatgctgccttatccaaataataaaagtaaattattaataagtcacaatttaatgaagattgatgttagttgatctttatattcttgaaatcagccatatggttgtgtgtgtatgtatatatttttaaaggtacataaagataataagctcatctctgaaaatttttacatttggcataagaataactggataattaagcatcttattctctggcctgtgtctttacagttaaaggtagatttactcacctctccttttttgtttttctaagttcatcttttttgctgtttcaagacagaggcccattttagctttctcgcatatccttttgtttgtactttggaagcctcacctgcttaattgttgagtttttatccgtggtcttttagagggggatatgtagggtagaagctttcacaggttcttgtttgcacttggcccctgactgttttgaggaatctccctcactgactcacagcatggcaaggtttcagatctctttctgccacacagcagttctgaggcagctggaaagatatccagatgcttagattgtcaggccaggcttgagatatacaaactattgagccttatctgtgaccttgcttaggtgaaggcatcagagcccctgcaccaacatgcataggcctctgcatgtgtgcggggctgggtgttgaggtctgagcacaagtgtagctggagaggtgagcttgatgtggcgacgggtatgagcaggttttcttcagacttctgtgagtttacctagttccaggatttaaaggcacagagactttagaattaaaatagaatcattttctttttctaaatagcaacactaggaataaaaaataataattccacattcttgacaggtaatgttttttcttgtcttctaatccttatttattccatactcatttttatacataattgaaatgtattatgcattggatttttcttttgcattatattatagacgatttttcatgtaactccttactgttccattttatatgttttgtctggtttaagactttatctgcaaaccgggaaactgtctctacaaaaagaaaaacaaaaatagttggccgcagtggcatgcgtctgtggtcccagctactcggggctgaggtgggaggattgcttgagccttgggaggttgaggctgcaaagagccatgatcatgccattgcactccagcatgggtgacagactttatactgtctgttttgggtgatttgataatgatatgccctgatgtagtttttttatatcttgtgtttcttgtgcctgggtttattgaggttgggtctgtggcttcatagtatttttaaagtttggaaaattttaggccattctttctttctttctttcttttttttttttttgagacagtgtctcgctctgtcgcctgcgttggagtgcagtgacactatcttggctcactgcaagctctgcctcctgggttcacgccattctcctgcctcagcctcctgagtagctgggactacaggcgcctgccaccacgcctggctaattttttgtatttttagtagagacgaggtttcactgtgttagccaggatggtctcaatctcctgacctcgtgatctgcccgcctgggcctcccaaagtgctgggattacaggcgtgagccactgcacccagctaggccattatttcttcaaagattttttttctgccctgcctccctccttttttccctctcttaaaggggctgtgatttcctgaatgattgcttagtgttgtcccatagcttactgatgctcttttcagtgtttgattgttttatgtgttttctgttttgtatagtttctattattgtgttttcaagttctctgatcttttcttctacagtgtctactctgttgttaatctgttaatctgttgttaatcctgtccagcgtatttttttttttgtttttgaaacagtctcactctgttgcccaggctggagtttagtggtgcgatatcagctcactgcaacctccacctcccaggctcaagcaattcttctgcctcagcctcccgagtagctgggactataggcacgtgccaccacacctggctaatttgtgtatttttattagagatggggtttcaccatgttggccaaactggccttgaactcctgacctcaggtgattcatccgcctcggtctcccaaagtgttgggattataggcatgagccaccgtgtctggcccctgttcagtgtatatcactaattttgtttttatctctagaagtttgatttaggtcttttaaaaatgtctccctgtgtttctgtttagctttgtgaacacaattgtaataactgttttaatatccttctctgctagttctaagatcttctaataacttcccagttcttggtgtttctcattggttgattgatactcctcgttttgggttgtattttcctgcctctttgtatggctgccaattttttattggatgcccaaccttgtgaattttactttgttggatgctatatatttttgtgttcccatagatcttcttgagctttgttctgaggttagttgagttacatatagatggtttactcttttgggtcttgctttataatttgtcagatgggttggagcagtgcttagtttaggactaattttttttttggactaattattcctctttaggaataattaggtaccatgcttaggaggcaagaccatcctgagtactctacctaatgaaccagaaagtttgggttttccagtccgcctgctgagaacagtgactttctagccctgtgtgagcgctgagctctgctccttctaatcctttccaatgcttctttccctggcctcagggagttttctcacacacatatctctgctgagtactcgagagggaccttccccagatctccagagctctctctgtcttgttttctcttctctggtgctctgtcttatgaactgtggctgtcttggtctccttagattctcagcacctcttcaattcagagggttgcctgtccctcctccttgtgccacagcctaggaactctctcaaagcagcgagttggggcagccatagggctgacttagtctctcgtctcccagggatcactgtccttcattgctcatgtccagtgtcttgaggactctgggttttgtctgttttgttttttggtttgctttggttgtctcaggcaggagggtaaacccagtccctcaccctcattgtgctcagtagtggaagtctcactctattacattagatattagtatttgtagcagagccctggttccctggtacttggggagctcttgaaaggccagaaacagcatgctttctcaccttttccagggcttcagtttctggtgcacatcaagcattccatacacatttgttaaagtcctttgttagacaagtagtgattcacaggttctatttgtaattttttcagttaacatgtattgggtatctgctgggagctagtaaaaacaaaaagtggtgtgtgacaaattcaattctgacaagaacaaccttaaacacttagaatatactttgagcatatcagaattttaaaaatgtgtggcccttgagtatttgaaaccaacaagaatctattgcttattagtagaggatattttgttaaacaagtggagagagaggcattttcagtctaattggtgttggcttttagcagctgatggaaaccagttcgtgattagccaggcagtggtgaaacaggctgtgcattctgaatgcctaggtatctaggcattcagaatggtggcgctctttgagttagcatcttcttctttcttgattcttttttttttttttttgagatggactttcgctcttgttgcccaggtaacaactccagtgcaatggcgccatctcggctcactgtaacctctgcctccctggttcaagcgattctcctgcctcagcctctcaagtagctgggattacaggtgtgcgccaccacgcctggctaattttgtatttttggtagagatggggtttcactatattggtcaggctggtcttgaactcctgacctcaagtgatgcacctgcctcgatctcccaaaatgctgggattacaggcgtgagccaccactcccagccccttcttgattcttgaaaaggacattgggtgctgtacatctcgttatagatgttgataaaaatgcttgtgagaagagtaacattaaggtagttatttggtcatttttgcagattattttaagacaattctaggactgatttgtggtaaatcacacattgctgtatcatagttgtgttcactgaacatattcaggggctctacagatgcagggctcttagctgctttgcacacttctgaattcctgccctgcgaacaggactggatacctaatagacaacaggtacttgataacagtttattgaattaatgagtgaatgaacagatacataaatgcatgaaagaatggttgtaatgtatataacttggatttcaagactttttactgactgttcaaaataagaaattgaaaactttcctctgattttcctctactatttacacaatttaaatggaagttatcttgtaccttcaatttctgtctaggattcgtacaataacgggtcatctctgagtcgcttaatgtctcacttgtctttctacagtgtgttgaagagatcctaggatacctgaaatcctgctttagtcgagaaccaatgatggcaactgtttgtgttcaacaagtaagagcttcattcttttcctcttctgttaagacgttcgggtatgacagcaaaacgctgctactccttaagaggcaggcgctgttggcataatcagctgggaggattgtggggtccagcgcagcactttttggctcagtccatgattgagccaagaggccatccttcccttcactccccaggaggacgaggtctgtcactgtggagggcagaggacaccagaagctcctctgcaacctcgctagttaacttccagtccctcggagtttctgtttagaatgctcaatctcatttagaattgcaaggaaacccaaaacgcctatttaaggtacaaacagcacttcatacaatatctcatgaggtattaatagtgattcacaggaagaatttcacgctgtgagtctttgctaacatatccagttatttacagatggatttgatatttgtgtgggagattcttaaaagtgttgttcacgccacattgttgatgcctcatttttttcactgtagttgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcggggtaacagttgtggcaagaatgctgtcgttggtggaagcacgaaagagcaagcaggaaatactttgtaaaagaataaaaacgaaaaatgttagcgaacatcttctaatagtctgctgtattcagagaactctaggagatatatatggttgatgcaaagatgatttaaggcatagcccggccttccaagaagtgtgtggccagtgagtgagatgggcttgggacttacacatctcagaggtgggggtagaggaggaggaacactgagtgggctgagaagcagccagctctcattgccaaagtgtgtcagcaaaccagaatgcagttcataatgtccccacccattcaaagcacaggacctgtagagtggtgtggcatgtgttggtggcacttttcaggcctgtaacaaggatgaaagaacagcttcatagcagcacagtagtgctggtgttcagaggtgtgtgaaggccatagaagcatcttggatatattaccttgtgttttgtcagctttatgactagaagtctcttttcacttaaatttgttttttttttttttgagacggagtcttgctctgtcgcccaggctggagtgcagtggtgcaatctcagctcactgcaagctctgcatcctgggttcatgccattctcctgcctcagcctcccgagtagctgggactacaggcgcctgccatcacgcctggctaacttttttttgtatttttagtagagacggggtttcaccatgttagccaggatggtctcgatctcctgacctcgtgatctgcccgtcccggcctcccaaagtgctgggattacaggcgtgagccaccgcgcccggcctcttttcacttaaatttatgtttgtgtttttaatgcctagtatacaggacttcttaaattgccttaagtatgaacaggtatttgagttgctaatctgtatagtagcaataatagaatcccttgtttttccttttataaatttagcgattaaatagctacaattaaaacactagagtcaggagtcaaggaaaatacccatgttccaggctgtatgttagtgatgtacttactatatattggagtttcaggagtaagtctgtttcaatgctttctgtaaccatttggggtattaataagcatgtgagtgtgtgcatgtttgggttaatttcatatatgtttcttagaagggatatcattgatgtaaatattttaaaggcttgtcctccaaaaaaatcatgtaatttcttctaaattactgatcttttaaatgaccttcacctttctctcaaatctcacttaagactgggctgagtagtcagtttcctgtagcagaaaaaagctcagacttgagtagccttctgcgagtgaggagacttgatggctgtcaggcagctgtaaactctaaatagagtgtcattatctgaagagggcgatgctgccacactgagtggcctttcaagttgtttctcaatctgacacgttctgatcgtgtgaatgtgaaattggtttgagcaggagtatatctgagtgcagaggagattatttaaagatattctcattctctgcttcccttttattcccatttggcagatggtttgatgtcctccagaaagtgtctacccagttgaagacaaacctcacgagtgtcacaaagaaccgtgcagataaggtaaatggtgccgtttgtggcatgtgaactcaggcgtgtcagtgctagagaggaaactggagctgagactttccaggtattttgcttgaagcttttagttgaaggcttacttatggattctttctttctttttttcttttttatagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtttgtcacttttatctttcatccatcatacctgttcctaatttagtacaaattaccctaaaagacactgaaatctactttaaagaaatgtggtctgcatgtttccctcatcagttgctgctgcttatctttttcatgcacctagctggtgcagaaggcctggggcatagccagcctcagcaagtcagcatccttgccccagctccctggactcaaggctaacctggggttggctgttagggatttccaaaggtttgtcccatccacttgcctcccctccaaaataagtttgaatttaaattgtgagatacaattaagatttattgtttggggaacatttttgcaaaatctagagttagtttaaacagattatcaattattaccataattgatcatctgcagtttcaagctatctaacaggttcacttacctctttaaaaaggaatggaatttagcaggacagtaactgagacccgtgctcctggagtccatgtgggagctgtgtggctctgcacaagcatttgcacgcttcccctcttgactgcattaccttcctcctatagttgctgtgggcaccagattctggctagtcctgtcccttcatgatgcacattttcctcaagattcgtcccagttaaatcactgcagatgaaactgccttttcatcgtcaaaatttaactgtcatttttgagccgtgatcttgggctactttcttatgtggggtaggaatatttgtgagttagaaatattacacttctctatttccttctagacgtaaatctgttaatcctgtcagcactgttactcacctgaaagggtctgtttccctaggagaactgagggcactcggtcaacactgattttccacagtgggtattggggtggtatctgcttgttttttttgttgttgttgtttgtttttttttgttttttttttgagatggagtctcgctctgtcacccaggctggagtgcaggggtgcgatctcggctcactgccagctccgcctcagaggttcacgccattctcctgcctcagcctcccgagtagctgggactacaggcacccaccactacgccaggctaattttttgtatttttagtagagacgaggtttcactgtgttagccaggatggtctccatctcctgacctcgtgatctgcccgcctcggcctcccaaagtgctgggatgacaggcgtgagccaccgcgcccggcctggggtctgcttttaatgaaggaggcatcaaggggtgggctttgcgttggcctgatgctttcatctttctttcacaaaacctgtccgaagaaaatccgtctaaatgggccattgctctcctcaggaaatagtcattgggaacttcttttcctttcctttgacactaggaggctgactggggagaagccctggtctatggctgtgggcagcaggggctgagaggagcaggctctcaggggggcacgggtaccccaagggaagccagagccctgatttgttccattctagtaagaacaaagactgctctggtttcatgtttgttctgattgcctttcatcaaccggtcccctttctcccagttcttaagattcagtacagtgacagttttatgaacaagaatagaacactagaacagacaaaccattgaactctatgctgataaagatttattgagctcctgctgtatgtttgcattctgcccagaggctctgagaaaaccaggccatatgctccatgctttatccatggaagctccccgtcaggttgggaaagctgacagctgcagggaatacagtgtgacacaaaactggctcccatgcagcccttacgtgtcgcctctcagatggttgggggacgaaggtcgactcctttgggtatcttattactaaaccagtttcagggaatctgtgccaccctatctgccattaacgtgaacagatgagtccccaaggtgtaattttgggtattgtctgatgtctcttggaatttattatttgtttttccaatgagatttcacctcagggtatagtaaagttgttgaggggattcctggatgtgttctgcaattatctaggctgatttcagaatagagttatgcttatagtcaaatttatcagctgtcaagaattttatttaaaatttatgcagataagcaggaggaaaagaagcctggtttttacattttaatcctattattgatgtgaaattttattttccttcctgtaggtgtttattggctttgtattgaaacagtttgaatacattgaagtgggccagttcaggtaatagcattttattattttagatttttttcttcttcttgtgtacttacatgtaatttaggttattaagtgaatgtttaaactactgttaggcatttttgctgttttctttaaatggaaatctgactaacatactgtgcatttttgcttctcttaaaaattaatgtatatctcaagacttgtttggaagtagttatgtatctgaaaattccatatgttgtcagtattcattgcacatttcaaagcatttaattgtgttgacagatggtggaatgaaatcttgtggtggagcactagtttttaaatcttcttagagaaagcagttttatataatgttgtctttagtaattattatgcatttgtattctctgcagctttttcttgctagatgttgaggttttaatacttcttgctagtccattacaggtttataattattaaaagttaaaattcttttagtacctaaaatgcttaataaacattgtaattaggaaaatttagtgcagaaggaaagtgttcccagattccctggggtctggaaacatagtgtttattctaattacatgacacctccactgtgttttggggcaagttactgtttctcttttgagtttcaatttcttcaagagcaaagaggcagaggagagctaggaagatcgtagctgctgtgcccctgtgccgtcgggtgccttctacctgctgcctccgaacctttacacatgtccctgctctgcgcgagggcacagatgggatgcactgtggcaggggtggggttagagtagatcacggacacctgttagcttgatgtgtgcttgctgtcaaggttgaatcatgaattattttatgttgcttatattgatatgtatcttaattttaaaagaaaggtctaaatggatgtttttgtttttagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatggtaacgggacacacctttcactgtcgtcttcggtgtcgtgatgtgcttggcagtgttcgttttcatatacccactttgaacgttgtcagtggcagccatgtgcttctcaggctctgcatgtgtgtctgtgtatgtgaaggtactggttagagacgtttcaaaagagaagagagcatattctttactctcagcaatttgtaatcttctcagggaaaaaaattcaagaaacagtaagataacctaaggtacagatagattctgaatataaagttcctgttcattcacatgaaacgctaaaagttcttcacttgatcttagccaaaaggccaagaagcgatgcaacactaaaaattcttaaatcgaacttgccgtgaattaaattttgatctctcatccagtggtattggagatatagtttgacttgggttcagggctttctgttttgcctgatgattttgctggagcttaaataaggaacccaggagatggccagctgtgcaagcccccagcctgtggaaggagctagtgtggttttatgaatgagttgcaaatctttctttgagctttttgaactgatcttccagcattgccctattgacccctccctgactcctttgctggaatctgtaggcttttgaactttgacagggacacatcctaagacccttgcaaactcccagatgtgagaatggcactactacttagagtcttttcgactcagcgtgtgtgcagaagagcatcaaccgggctgtgttgcgaggcagggccttggctgacctctcagtgtttacatagctaagccagttagtgtttgccacggcctcacaagggcttcagattcacacagccaaagtatagattattaaaggcataggtgtttggtttcctggacttggagggtctttggacagaaaatcagtaggcaaccacacccagtactttgtgctgggaagcttggtcatctgtgagagggtcagagagtatacccatgcgtgcatgccaccgaagggtcagtgagtattcctgtgtgtgcatgtctcagggccggagagagtatgtgtcactgagaggtcagagtgtttgtgtgtgtgtcaaagagggttgcattgtgcccttcactgaggggtcagagggtgcctcgcgtgtgtgtgtgtgtacgtgtgtgtgtgtcactgaggggtcagagtgtgcctgtgtgtgtgcttgtgtgtgcgtacatgtcactgaggggtcagagtgtgcctctgtgtgtgtgctcatgtgtgtgcatacgtgtcactgaggggtcagagtgtgcctctgtgtgtgctcatttgtgagcgtatgtgtcactgagggggtcagagtgtgcctctgtgtgtgtgctcatgtgtgagcgtatgtgtcactgagggggtcagagtgtgcctctgtgtgtgtgctcatgtgtgagcgtatgtgtcactgaggggtcagtgttcctatgtgctcatgacattgagggtcagagtgtgcctgtgtgccaatgaaaggcatttcttatatttttttatatgtggtcatagtagaccagttaatttattttgactcctgtgttagaccaaaataagacttgggggaaagtcccttatctatctaatgacagagtgagtttacttaaaaaagcataataatccagtggctttgactaaatgtattatgtggaagtctttattgtcttttcagatgaatcaagtagattattcttgagaccaggaatgttgctgttttggttatttggaaagttttatcattttcaaattgacttttgaatttgagtcaccttttttcagaagtggtgttaaattataggagccctaggttttttttctttttttagaagtcatcacaaaatgatcagtgttcagaggaagagctttgaccttccacatggtataatgattgataaccttaattcatctcttaccataaaccaagtatgtgtaagggttttctttatttcttgaaagcattttgtagatgttgagagcagttttccaaatgtaatttccatgaaatgcctgataagggtacccttttgtccccacagccataccggctctgcagcccatagtccacgacctctttgtattaagaggaacaaataaagctgatgcaggaaaagagcttgaaacccaaaaagaggtggtggtgtcaatgttactgagactcatccagtaccatcaggtaagaggaatgtatgttggaactgtcgtggatactttattgacccgtgcagatggaaggaagtgccatgtggtaacgctcactgttaactgtgttactttgaaccaggtttgggctttctggggcctgggtagatgccggtgcagggggatggggagggaggcggggggtgggggggtgtggtggagttggggaggtgcagtggcaggaggtgttgttggtgtgtatcctttttttttttttgagatggagtctctctccgtcgcccaggctggagtgtggtggcacgatcttggctcattgcaagctccacctcccgggtttaagcaattctcctgcctccacctcccgagtagctgggattacaggcatgcaccaccatgcccagcaaatttttttttttgtatttttagtagagatggggtttcaccatgatggccaagctgtttcgaactcctgacctcaagtgatcctcctgccttggcctcccaaagtgctaggattacaggcgtgagccaccatgcccagcctggtgtttatctttaaagtgggcacagccacaggagttcacctgactcctggtctgagagtcacgagatcgttcaagatagtgaggccctcttttccaaaacgaggaccaaaaatcaattgacagtgttggtcaagatggtagaaaccttaaaatgatagaaatctcaactctgaaataaaaactttatttgtatatttatttaccactattttgacatagggctaaggtctttttctttgagctgatttctggttttgttttcttaaagtggcataagaattcaaagacattttgaggaaggctgagtgcagaaatctctctttttaaatgacttctcctttcttttaacttgcactgttgtctagccctcacttattttgtcaattctttttagctgtttgtctttgaatcttcataaagccatagcttttctcataagaagcagcactttctttgttcattcatattttaatgaacccctgtagtatttaattaaatacttaatgcctaattaaatcacataattgcaatgcaaaagtacatgtatcataaagaggtctgaaaatgagcaactggcaagcaggtggtggcaggcagagctgcttgggtgggtgggtgtcatggagaggagttcatcagccacatgttcagtgagctctggatatgtctgtttagaaatgatcactaataaacttgtgctcaaccatgtatacctctgggaagcaggtgctcttcagtagattgcctctgcagagaacacagaattgaagtgaatgtccacaaaggcaatgagccacctgcagaatagtttagtcaaggctgtgtttgaagtttgccaaagattaatatacatttgattttcatgttgtgccttttctctgattgtgaaatattacaaattctatacaaataacaatgatggcaaatcctcctgagcaaagtgtgcaccttgtatgtgccctagaggaacttgtgtttcgttctgattcccctacatttctcatgtcatagagtgggggttgcattagtgtccccctgtcctcgctgggatcacatctgtttggatcctagagtcttccagctgaactgggacaagtataacagacggacacgtaggggtggaaaggcgtctcttggcagcagactttctaattgtgcacgctcttataggtgttggagatgttcattcttgtcctgcagcagtgccacaaggagaatgaagacaagtggaagcgactgtctcgacagatagctgacatcatcctcccaatgttagccaaacagcaggtttgtccccgcagccttggcttgttgttgcatagtgatggtagcttaaggtccttgtgaaaggtgggtggctggaatcagctcttccttcagtcctaatctgtgccttgatagcagttctccgtgctagtcatgggacagctgacttcatttcttctcacaatgccatctcaggttggtattgcccacctactttacaggggggatcccacagctccgagaggttatggaggtgatcaggcagcacacagctttagagtgctggggtgagggcgggccaaggctaactctaaagcccgaacccttacctcctacactgcctcctgcattctggtcaacccagtgttttatttggtggttagatttttgtttttgttaccttactgcttgtaatttagcagttttcctttcctttcccttcctttcctttccgacagggtctcactctgtcacccaggctagagtgcagtcgtgtaatctcactgcaacaacctctgcctcccaggttcaaccaattctcccacctcagcctcctgagtagcaaggaccacaggtgtgcaccactacgcctggctagttttttgtatttttagtagagatgaggtctcgctgtgttgcccaggctggttttaaactcctgggcgcaagtgatccaccaaccttggcctgccaaagtgctggcattacaggtgtgagccacctcgcctggcctattcatcactaatcagaatttctatgatcaaatgacatgaatcattgtttccacaactgcagtggaaggaaatggcctggcagtgccagtttcagaagcagcctgcccccagtcaggcacaggccactgtgcccccagtgtagcagcacctctgtagctcacagagaagggtggtggggacctccttgaggcagctctgccagaaaatctcatgagctgcctggcacagcttgaggttgccttttaagtggactcagcaaatacatgtttgttcatcttgattatacacaataaacaactactctgtatagtacgagtagtccgtggtttttggcatttgatttaaacttagaggcatgtgatattgatgttactgccttcatgactgcacccccattctgatttcataatggaatgttatcttgagaccagttagacaacaggacagggatcttggcttctggtgagattgacagcagttttagtgtggtcagggtctccctgcctacagatggttttagaatggtgccctggaagctttatcccattcttttctgtgcgtaatctgagtagagtggagatcgaaggcctgaatacatagtaaatacctgacttaatatctgccgcaatggaaattgtgtgatacaacatttatgaaacgcttagtgcagcacctgccaggtagctcaccacaggtgcatgttgcattcagaagtagtgctagatactatcctgttactggcagtgcatacatcagtgatcaaagcagattaaagaaagaccccctgccttcttggagtgaagattttgttgggatgcgggtaaggggacagacaatagaaaagcaagtgagtgaagtctataccatggcggctgatcaggaacaccgtacagaagaatccaggagggaagagagttaggtggtgtctgcggtgggagtggcattgttcagctggtgatgagaagaagctttggtgatctggtgacatttgagtgaatttgcagaaaggaaagatacaagcctaggagatacctggggaaggaacattccaggcagagcaaatagcagtgcaaaggccctggcggggggcggacatgctgttagggtacaagcaatgagggtggaggagtggggcagccatggggagggaagggagtgaggcctggtggggtgaggccagtgtggaggagccttgagagggtttgcgctgatgtggtgtaggttttagcaggatcattcttattcctgagttgagaatagccttgagggggaggtgagggcagagcagggccacccatgtgagacccggcactggagtggaatggcccaagtcagcatcccttggcagcatgaaagcaaaaccagcaaggtttgctggtggcttagatgtggcatgtgagagagagcagggctttgggggtgatttcagggtgaggacagggtggctgtggacaaggtagggcagacattgggggcagcaggaggtcagagcctgtctggatgtagcagttgagaccccataggtgcctaatgaggtgaggccagcatcaggtgtatgagcctggagttgtcgagagactgtggggcagggggtcagcatctgagatgtccactcacagtggacccagactggctggagaggaggaggagcttgaataccgagcctgctgagtcccagctccaaggtcaggtaggtgaggggagccagtgctggggcagggggagtaggcaggtgtggggttcctaaagccaagattttttttaaggcattttgtgcaggagggcgacatctgctgtcagcaccttgggaacttggcccaggtttggcagcaccgagggcactgatgagtgcttttggaggagcaaagggagccaaaccctaatgggaatgtgttcctgaaaggacaggagagagacttgggaaaaggttttacttgaagagggaacggagaaatagggcagtagccagaggaggagaggagtcggcaatgggttaagttggcagaaatgaaggcctgtttacgcactgagggcagaagcaacagggaggatcagttcatgacacaggagacacaaatcgccgttgtggtgttcacagacatgggttaggattggctgcatggatgacagagcactgtgggttctcccagagttgctggggaggaggcagagttggtgagcacaggcgagggtccaggatgcaggaatcctggagctcaagtcagttgttcccttgttgtaagatgtggccagtgttgtgagcttcacatctgtgccttgaaaaacaccacatctgtttgcagagttgtttactatgtatacacactcagtagaaacaaaaattggaaacagtcagtgcccaccatcaataagtaatggttgaacacactgtggtataagcttagactattttagcttgggctattttgcatgattaaaaatgttctggccaggtgtggtggctcatgcctgtaatcccagcactttgggaggccaaggcaggcagattgcttgagctcaggagtttgagaccagcctgggcaacatggtgaaaccctgtctctactagaaatacaaaaagtagctgggtgtggtggtgtgcgcctgtagtcctggctaactcaggaggctgaggtgggaggatcacttgagcccattcgtgcgccactgcactcctggggcacagagtgagactctgttagaaagagagagagagaaagaagagagagggagggaggaaggaaggaaggaaataaatggaagaaatggaagggaggaaggggagggaggaaggaagaaaggaagttcagccagttgccttgggagttctccattgcactgggttaagtgagaagagcagagacgtttatgatttttcaaaacaactaaaacaaaacctctgtgggtgagggggcaaggatatggctataggaacatggggcagattaagaaagggatatacacacaccacttagcatttgttacaactgttgtgggagggatggagtgcagaaaaagaaaaaaaaaagtgcacaccatcccatgtatgtgtatacaaagggacgcttggaagactggtccccaaaatgttggtaatgattgtgtcagggtgctgcagtgctagttgattttttttcacacttttgtatatttgagtcttttacagaaagcatttattatttatgtaataaaaatctaaatgacaagatttctgttatgggaaaaatgtagctatacagtgttgttgtaaaaatgtttgcttggttcaccactgaacttaaaatgcttttaaatgagggaaggtgacgatgagatgattatgatgatttgcccttgagttacatagctggtgtacaggaagctgtcgtttcttttggcttacgtagaaatgtttgtggtgtctaattccacagatgcacattgactctcatgaagcccttggagtgttaaatacattatttgagattttggccccttcctccctccgtccggtagacatgcttttacggagtatgttcgtcactccaaacacaatggtgagtctctcgcctggctcagcagatgaatctggacggcttgttcaggctctgattactgggaccacccccagaatgtctgagtcagtcagtttgggtagggcttcttgagagtttgcttttttttttttttttttttttggtgtgggggtggtgcggaacagagtctcactctgtcgcccaggctggagtacagtgtcatgatctcggctcactgcaagctctgccttccagcttcacaccattctcctgcctcagcctcccgagttgctgggactacaagcgcccaccaccacgcccggctaatttttttgtatttttagtagagatggggtttcaccgtgttagccaggatggtcttgatctcctgacctcgtgacccgcccatctcagcctcccaaagtgctgggattacaggcgtgagccaccgcacccggcctttttattttttttggagatggagccttgctctgtcacccaggctggagtacagtggcgctacctcgactcactgcaacctccgcctcccgggttcaagcaattttcctgcctcagcctcccgagtagctgggactacaggtgcgtgccactgtgcccggctaattttttgtatttttagtagagacggggtttcactgtgttagccaggatggtcgcgatctcctgaccttgtgatccgcccgcctcggcctcccaaagtgttgggattacaggtggctctcgcaccaagccaagagtttgcatttttagcaaattcccaggtgaaactaatgcctgcttttctgggagcacactttgggactcagtgatagagaggtttattggtaggatagtaaaataggagttattttctttcacaaaattggcaattgggggaaatttaatcttccttttttcttcagctgtgacttatgtattatgtttattttaggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtatgctttctatctgagcctataactaacccatgccttttgggaagtcacgtgatgtttcacagtcagtaagtctggaataatacctggtcttgcttcacttctgagttgggtaaagaagtctgtatcagtgtaattttctaatccgtcctgcattatctatggctcttggttcatacctgtcttgaagttctgtcatgttctgtctcttgtcctcagtagagatgctacagcagtggctcgcctcaggcagggcagggcagtggggtggctgtcctgggggcaggcagtaggggcacgctgacgtcagggaagttgaaacccaagagaagccagtaaaagtgagtctcagattgtcaccatgtgctggcagttttacacgctgtcagtaataaaagtcttctccctgcagggcagcctgcctccaataaatacgtgtagtatcaaatcctgtcttccctcataaattgtttggaagctccccaaggacagtgatgaggcactcgtaagtgcttgctgcctagatgggtccctctccacctttgctagattctgagcattcactgagttagagctgcttctgcaaatgtgctgcttctgctaagtggctgtgacttcatgcagccttcacttggtttgtcatcagtggagatgccctgtgttgtcgaaggagataagcccagtaagcctgctgggcaccttttggtttgcaggttcagcaggcagcccatggctttccctgtgtcgcattgaagcagctggctaaaattgatgatacattaaattcctgtgacagatgatcagcttgtatttgtgtaatggtgtacagttcacaaagcttaaaaaaatgctacctgccatttcatcctcagtgaggaaggtgatacacagagagaccaagtgactgtgtccacggcgacggcgctctgcatttcactttagcggttaatgtactctacctatatttttactttatatttaccatatatcttttcatgtatacttggcgtaagtgctttatagtagtcacctaattcactgtcatcttttttgtttcttggaaggtttctattacaactggttggtattcttttagaagacattgttacaaaacagctgaaggtggaaatgagtgagcagcaacatactttctattgccaggaactaggcacactgctaatgtgtctgatccacatcttcaagtctggtaggtgaatcacattagtcttcctggagtgtctcgttccccattctgcactatacactctcagagtgtaggagctgtgctgcccggtagaaactctgccttgcccagtgtgccagttgaaaatatttgttgctgtaagagtacacctgataccatgtgacccagcagttccactcttgggtatatacccaaaagaatggaaagcagggtggtgaaaagatatttgcatgccagcattcatagcagcattattcacgatagctaaaatgtggaaccaactgaagtgtccctcgatggatgaatggataagcaaaatctggtgtatatttacagtggaatattattcagccttaaaaaaaggacattctgacacatgctacaacatgggtgacccttaaggacattatgctaaatgaaataagccagtcacaaaaggacaaatactatgtgattccacttacatgagggacctggagtagttaattcatagatatagaaagtagaatggtggttgccaggggctgcaggggaggggagttatttttacaagatgaagagagttattctagaaatgaatggtggtgatggttgtataacattatgaatgtacttaatgctactgaactgtacagttaaaaatagttaagaggaccaggtgtcatggctcatgcctgaaatccaagcactttgagaggccaaggcaggaggattgcttgagccaaggagtttgagaccagcctcagcaacatggtaggaccccatctgtacaaacaaactagccggggatagtggtgtgcatgtggtcccagctactcaggagactgaggctggaggatcgcttgagcccaggaggttaagtctctagtgagatgtgttcatgccactgcactccagcctcggctatagagtaagaccctgcctcaaaaaaacaaaacaaaacaagacaagagccaaaaatggttaagatgggccaatcacagtggcttatgcctgtaatcccaacactttgggaggtcaaggtaaaaggatcacttgaagccaggagcttgggaccagcctgagcaacatatcgagacccctatctctacaaagaaaatcaaaaactagctagatatggtgggcacatgcctgtagtcccagctacttgggaggctgaggtgggaggatctcttgagctcaggagttcgaggctgcagggagctattattgcactccagcctgggctacagaatgataccctgcctcttattaaaaaaaaatccaaaaaaaaaaaaaagtaaacctgagagcttcctcctcctgtgttaaatttggaggccaagatgtttttgttacttttacaaatgatcaaggacggtgaaggttgggcatggtagctcacacctgaaatcccagcactttgggaggctgaggcggggtgatcgcttgagcttgagaccagcctggacaacatagcaagagaccccatctccacaaaaataaaaaaataaaaaaaaatagccaggagtagtggcatgagcctgagcccaggaggtcaagctgtagtgagccatgatcatgccactgcactccagcctgggcgagatcgagaccatgtctctagagaaagaaaatgacaaggacagtgaacccaagaaagtcataagatgccagctgtgcagcaagcatggaaagcagccagtccaaattaggacagtgtgttttccaagaagaacgatcgtttgtaatgagaatgctttgctttaaataaatgactaaatagctagaagcctagttctaggggataggcacgtctttcttctctcaagaaaatagaaaggcaattctaatttctagtaacagcaaacagcattaagtcatggtccaaatatgaggcaaaccaaaatgtggcttgattgttcagcagttgatctgttggaagcccttgatattaaaaaggttctcctttaagcggcttaggagtcacgatcaaagacctatagaaagagatgccatccttctaggatccttggctctcttgggaactagattcagatagtcataatgtaaatactgcttgagctttctttctttctttctttctttctttttttttttgagacagagtttcactcttgttgcccatcctggagtgcaatggtgccatctcggctcaccgcaacctctgcctcccaggttcaagcaattctcctgcctcagcctcccgagtagctgggattacgggcatgcaccaccacgcctggctaattttttgtatttttagtagagacagggtttctccatgttgaggctggtctcgaactcctgacctcaggtgatccacccgcctcggcctcccaaagtgctgggattacaggtgtgagccaccgcacccggcccgagctttcatttttgaaatcaatgtatgactgaaacactgaagacttactgacttaattatggtttcagaacagaatgaaaatgtcttcggttctgatgaatataaaaggaaaactaaccaagttaatttggcaagtagatggtagagatagaggtggggagtggaaggggaactaaaatcttcacctagcattgttgggattatatggttacatcatctgaagttgacagaccaaaatatagaggcttcagaggtctccaaatagaactaaacatgtaattcagattgttaggaggtagtataaatgagctaaatctcatctttattacggtagagttaatgggtgatgtctaaagttgtctgaagtctataaatcatgacaaattatgatgtggtgattgtattcaacagtctttcagttgcagggataaaaccccagtttaaactagagtaagagaaagaatgtgttggtttaagctcctggaaagtgcaggcaagggtagttggtaggactgcatctagtgttgtaattctgtggtctgcattgtatatttatgcatctcagctctgctttcttcttttcatttatataatttttaaattttattttaaagatagggtctcactttgtcgcctaggctgaagtgcagtggcatgaagtgcagtgcgaggctcactctagcctcgaactcctgggctctagagttcttcctgcctcagccttctaagtagctgagacaataggcatgtaccaacatgcctggataggttttaaaatttttttgtagaaatggaagtcttgctgtgttgcccaggcgggtctttaactcttagcttcaggcgatcctcctgcctctgcctcccaaaatgctgaggttataggtgtcacccaccacgcccagtctcatctctgcttcctgtgttagttttgttctctggtgggctgttttcacatgaccgaagatgacctctagcaggctgtgttctcagcccctcaagtaggcctatgtgattggccttgcatgagtaatatgggtgaccataaacccctgaatgctctggtccacatgggccaaatgggagactggacagcattccattgatgaggaggtggggctggtctccgggagtaagggagaggagcacatgcagtaactgatggtctgctgcaagggatagcagcacagcagttagaattttggaggtaactaccagaactgaaaacagaaatgataacaagtagttgccttaaaaagggatgggagcagggtgcttttgtgatcaaagctcctttctcttactggatttttgtacacattttgcatacatatcttagagtaaaagatagcattttcagccttggtccatttgaggatactcttggcgtggcccgcctccatgctagcaggctctggttgtgccaagttcagttgagcatcctggctcttgcctgcacggaacttccagtcagtgcgtcagtatcacaagtcttgatatttcctatgaagaagaacagtagtgcagtgacagacgaaatgggtgggcaggcagaggcaggatttctgagggagagaagtagctagctttttgcagagaagagttccggcacccaagagagcagctgagagtacaggcaggcaggcaggatgccggtagggcccggccgcacggcgccacagaatcctggagaaaggggcctcttcatggcctctgcattcagctgctgtcaccctccgcacaggccatggccaaaatttaattttcatagtggactctagtttttgagccttacttgctattattgaaataattttcttgtttctttttaaagatcttcggattatgcttcactgaccactgtaataagtttaaagttgagaaaatatggcttgttaatgaatgataggtcaattttagtatgttggtcattttaatattttgccaccagttggtttggatttgatgccaggaggagacagcctcatttctaaggactagtcttgcctttgtgggataagggtggtgtgttctgtgtccttctacatgtccgagcgatctctgtgcagctcaaatgtggtcactgtcttattgcgctgatttcctctccttccatctcacaattgaggcaaaatattgttactgttgaagtgttgtccaataggacttccagcagagacaggatgtctgcactgtctaatttagttgcctttagccacatgtggtgttctgtacctgaaatgtggctggtctgattggatagcttaatttataattttatttaattttaattaacttaaatttaaacagctctgtgtggatagtggctcctgtatgagacagtgcaggtctgttgagaagcagctttactggtgggagtggagggcttggagagggcacgtgggtttcctgctggtatcttttgaccttatttaatctgcccaacatttgcaagtaagttgtgtgtgtgtgtatatataaatgtgtgtttctgtcttcttgtttcctttgactgcatttatttgaaagacactaggtggcagaattactgtatttgattggtttcaagataagagttgaaataattcatctcgtgtttttatataagtaaggtgtgtttagcatgtaaaattggtaatatgtattcacgtactgcttaaacaaaggctatgaattccacccataaaccgaaaatgaagacctttaaatttgtccatttcaggcgtgggtacttcttaaataatacctggttcaggaactagtcagaatggcacccttgactttttgtttcctgcttttcctcttgttgggagaggagggtattcatcccaaagtggtttgcctatttcacattccatctaggataagcagaatagccaagaaagatagctgtcctcctgtttacaacatttggggtaaccagcatccctctcttttggtccaagatagactggtttagaaacagatgatggcaccagaggcccaggaggtggaaacatcagctttgtttgttgtccatgtggctgaattagagctgtctggccttgtagcctcaacacggccttccagctttgctcaccgtgattttcaaggacacatcttgtgctcttccctgcctgccatccagactatacccagtcagggtggcaggagctgctgccccttcctccctgagtcctggtcgtgggtggtggagatgtgccatgacgctcacggaggcatgctcaccccttcctctgtggcagaggggatggctgcacgacagctcttccctgtcctttccaaagcgtctgtggttccactttttggggcaaagcaggaatactggaagagagagaaagtggtcctttctatagtaataaagttgacattgattcaagttcatgcttggggaaaggacagggctactaacaattataatgctgggagcaatggaattttctcatgggtatgtggtaggtttaattttaattatcccagttaattcttagaactgctctgtgaagtatttcccgctttgtgcttaagttctaaaagatcctgtgccaaaaccaagaatgaaaacccaagcattctttcttgcccatcgatctttctctcatcaggccacttcttgggttgatagtggtgagtgtagccgctgccactttcagaatacccaccatgggccccagtcactgtgtggcgtggagaagagatggttctctctgtgtcatagctgaacaagcccagcccagagaggtttctgccctaggagctctcgatggtggaattgggatgcgatcccacatcctgcctgttttgaaaacagcattctttatttccaattcctgcttccattgttccttttaatatttctttgtttagctcacaaaaacacggcttgcggagctgctgcgtgcagctgtagctgtttctctgggtgcagcctgcatccgccttcctgcccgcctcctttcctgcactgccatcgtggtctccgggcacttggtccctttctcttcccctgagtccctttggctcccctgtgccacccttgtgatccacaggctctgccttctttctgtctcagactgctgctcatcactactcgggaccctaggaagggaggttccaccgagaagcatcttctcatctcagccacgttctcagtgccactgttgtctttgttaggtaatggtagctactgtaacaaataaaccaacatttccatggcttcacaccagagaaggttgtttcttggttttatgacaatgtattgagggtgttcttggttcacggatggttttcctccatgtgggaattcggggacccaggctcctttccttcttttggttctgttctccaggccttcacatcctctgtgtctggttggggacaaggagagggaaggtaaagaaggctttgtggccttggataagtgacaggcatgcctttgctggtgttctctcgtggtgacaggtcacagccccaccctgtaaaaggggactgagagacgtcgtcctgctgcttcccagcagcagcactgtggtctctgatgtgttttctgtgaggataaaaacaggtgattccaggatgaggaaagtcagggaaacccttggaaggaggggaccaggcgggtgtcaccatgggattagtggtggcttcagaatgagctgcagcgagtgccatgccttctaaagcttttgctattctgatatgcccacaccatgcccagcaggtgtctgccttgctctccgcagagagagtgatgaatccttctcatgagcctctgtccagttgttcctccctccacctggaagggaccctgggttcctcataacatcccagcggaacaggggaccttctatcctgtccccaagttcatcctcatcctcctgccggcttcctggcccctcttatgtctgcttcctgacgccacatccttctggattctctggaattgaattttgcctttgatgcttatttaaaaatatccattgcaggccaggtgtggtggctcacacctgtaatcctgtgcactttgggaagccaaggtgggcagattgcttgagcccaggagtttgagattagcctgagcaacatgttgaaatcctgtttctatagaaaatacaaaaattagctgggcatggtggcgcacacctatactcccagctactcaggaacctgagacaggaggatcaattgagccccggaggccaaagctacagtgggctgtgatcgtgccactgtactccagtctggtcaaacagagtgagaccctgtctgaaaaaaaaaaaaaaatccattgcatacttcaccgtagcgaaacatgtatgtcttacctttcctttcctgcctgtagctgctcttttacacttaacagccacactaagccagccttaaatgaaaaacaaaccagcacttcctgtgccctcctgcttccttcatgaggggtccctccctctgtgtacactccattctcattgcccatggtggtttgtttccctcttgtttctcaagccatggcagcctgcctcttgccctctttactaaaaaggcctttgcagaggctgcctgtgttctttctttctaggtctctctcatcctaggccctccagcttgattctgtggagctgccctcttgtcactcagtagcttgtggggtcttctctgtctagccacttaattgattgtgttcctcgagttgctgtccatggtctctcgttactgttttctctgtgtttctgcctctctccttggccttggtaggtccatcccctttgtgaccttggctgttgctctcatggacaactttctcttgctggtccttgtagtcctggcatccagcttctcgacacgggacttgtcctgccagtacctcagacttgcacttaaaattgaactagcaccactgtcactctccagggcctcttcttgttaattagatcattagggatgttcagaatcccagcatcatagtatgttcctcctcccgctaccccaggaaccctaaccttacctcctcctctctatctactaggaggtggccctcagagtccgtctcatcttccacctgaacttccctaataggctccagcagctgccaccccgggggctgagtacttcctccatgccttgtgcagtgctgagccctttacctgggttctcctgtttgctccttattacagccctgcgaacagatactgctcttaattccatcttacacctaaggaagctgaggccccaggtaaggtgcatccaaggtcacccaggtagtagacagtagagccacgatctgaaccaggcagtctgattcagagcctgtgttgacactcagccacctagaacacagcttggattgtgggtttctattacctgttcaaaacccctacatcccgggtctgtccctgcacgtgctctgtggcctggctgcatcttccttgaaggcagtgcatgcctcttcactcagggggcccatgcaggaacagagggccccacagaaggatgaggccagtgcagaatgggctggaggggacaatgctgaccaggaagcaagtgtagagaaatcccaggaaacctggaggagccagagacaaggcattagaactcctcgtcgtgacctggtctgcattctctgagtgtgctgcttctgttagctcgcttccttggtctcaggttatagtttaaggcattgtggagccctaaaaagcctgtactctgtttttacctgttttaggaccctttcactttggggatgtgttgattttttttttttttttttttttttttttgagatagagtctcgctccattgcccaggctagagtgcagtggcacgatcttggccactgctgcccctgcctcctgggttcaagcaattcttgtgctcccgcctcccaaatacctgggattacaggcacccgccaccacactcggccaatttttgtatttttagtggagacagggttttaccatgttggtcaggctggtctcgaactcctgacctcaagtgatctgcccaccttggcctcccaaagtgctgtgattataggcgtgagccaccacacccggcctgaaatttaaatcagaaataaaattttgatcccaacagtgatgccaggcagcccagatctgggggagagggtggccttggccagctgggcctttctctgtttcccaagtcttgctgcctctccctgctgggctttgcagcctgtgcatgtctctgtgcctttgaccttgtttatccaaaggagaggatagaatgaagtcatgattcctggagccctgagaaggatgctgtggagaaatttgccggtagaatctagctgagtgtgttgctgaggtgccagcattgtgtgtggggaggctgaccgcttggcctgcctaggcccaggatgctccatggccgggcacagaggccacttggctgtcaggtgtcaggagcctgcagagggcacacagagcctggaccgcaggggggtcctgctttctcacctggcctccttcagcatttctgtccctcagtccttagcaagcccaggagctgttgagtttggcaggtgccgagtgctgttcctgcctgtgtagctgtggctcagtcctgtgggggccccgctgtggcccgagtgcagtgattcgaggcgctgagtgttccctgactccttctccaggagctgtgttcagactttcgcagctcttggcttggagctcctggagggcttggcattgccgaccaatgtggaggtcgacagtgagagaggaggaatgctagctttcttgaccagtccattaaataagtgggatattggccaggcacggcggctcacgccttaatcccagcactttgggaggctgaggcgggtggatcacgagctcaggagttcaagaccagcctggccaacatggtgaaaccccctctatactaaaaatacaaatattagctgggcgtggtggcaggcgcctgtaatcctagctacttgggaggctgaggcaggagaacagcttgaaaccggaaggtggagtttgcagtgagccaagattgcgccactgcactccaacctgggcaacaagagcaaaactctatctcaaaaaaaaaaaaaaaagtaggatatctgtttctgcttagaaaaatcagaattttctaaatgccaggtgttctgaatacgtaagtatgggagacgactcagcctgtttcatttttatgtaaaatcttcgcgtagccatgtggcactggaccgagatgaaagcaaagacatttctccttaactttgtttctaggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaagtaggttcataatgccccacagcccagggcgccagcccagcaccctgtcctgagactcccagtaacctgagctttggccaccgttaaagcattttcattttccattttttgtgagggcttgtgaaatttctgctgcatattaatattcctttcatggacagcatattattgggacaaacatgcggtccagctaaaggcattcaaaatagcagttgctttctaaatgcgattttctttggcaggttctttgacaccattgcatcttgtgggatatgcttgtcatgctctgtggctcctactaagttctagtccttaaattggttccatagccagacatgttgcaatgtcttaacctcattataaagtaaatgtggttctggttatccttagataatgaagtaacagtgtagcaaatttcaaaacctcttggaaatgttattttaccattcaaaaaggcttactaaggttctcgttatgggtggccctctttttgcaaaaggttttcaggcttaagctccatttctaggtgctccaacactccattatttgtatatgtatggaaataaaagctgtgaccacccccaaccctggcccccgcccagctgaatcctcagcacagtatttctggaaggctcaagatcccacgctggggaaaagaagttctggagacaaaagagggcaggtgctgccgtgcctctctgctcagtatggatactggaccttgtgctgccagggctcccagtagggccagttcatggcactcagctggaaagtccactgttgggaggcattcttaaccatccactctgtgccgtatgtagtggggtctggtcattctgttggaggagacagaccagtgacgacatttgaaatgcttggtggatgtcttaggcctgttacgatgactgagcactgtgggggcaggagacagaaagtcagtgtctcctagttctgtgctgctttaacgtgcatagaaatcagctgcggattcagcagatcactccttttctgacagatgggcctgcttactctgatgttatatcagaaagctctgaatctgggaattgtgtcccctgaattggagtaacagaaatgcttagatgatgagtgtttaaaagaaataaaccaaaggtaaatttagtttggaattcagcaagcgtcttcattcagccctctgagggcaaactacagctttttgtaaatgtaggtaaattctgtgactgtttcgtgaccccctctgatccagttttcctttataaccttctgtattgttccttctattatcctgaaataacattaatagattaggctgggcgtggtggctcatgcctataatcccagcaccttgggaagccaaggcgggcagatcacctgaggccaggacttcgagaccagcctggccaacatgatgaaatgctgtctctactgaaaataacaaaaattagccgagcatggtgacaggtgcctgtagtccctgctactcagaaggctgaggcgggagaatcgcttgaacctaggaggaaaaggttgcagtgagctgagatcgcgccactgcactctagcctgggtgacagagtgagactccatctcaaaaaaaaaaaaaaaaaaaaaaaattaatggatcaatggatttttaacctaataattaaatttcaaaaaatatcgttctttaatggtaatgtaaaggtaaaattaagataatatgtaacaagcatgtgagtgtctaaggtgtccccgtggtggaaggaaaaaataaatccccataagtgtccaagatgcccatagagagcagagctgttctggtttaaacccctgctcttagcactgtgtttttccagctgtgggtggtgggggatgagtatctttttatttccatgagatgagaaaaatgaattactagaagtgtgaaatacaaaacacagctgctctttttttagccatagactcagcagccataaaattgctgtatccagttgcagaaattcctgctgcttactcttgaccctctctcggtttgtgtgcatctcctctcaggctggctcccagatgggagctggctccaggcgacactgggtgctctgctccaggaggtccttatgtgggtcctgccctagcctagcccctctcttatggactctgtcactgtgggtttatgattcactctcaatctgtcttacctcttggtgaactgttagagtcctgcctatactttggcgcttgtgggtgtgttgtggtacacatgatgtgttggtcacttcccagctcatcttgttctgagtcaccctagatttgggacattcattcgccaccagtaccgggcggtgtatggcctgagatttgggggggcttgtgctgctacaaattggggctgaatttgagttgacagtggaccttctttatgtctactgctcatatttgaattgcaaatactgcctcttctctttcagaggctcattaccctatagctgtattattgcaaagtgcacaattacagcttgagtgtaagtcacactgcgctggcaggacggcccactgagaaagggcacgtttcctgttcgttagttttcacattgacacataatttacaatacagtaaaatgtacttttctatcaactgtagtcagtaacagcccccctcccccaaccacatcaagatatagaggagtgctgtcacttcaaacagttccctcttcctctgccacatcctgcccctccccaggtctaaccaccaatccgtgctctgtccctctgttcagcccattgcagaaggccatagaaatagaatctataggctaggtgtggtggctcatgcctgtaatcccagtattttgagaggctgaagtgggaggatgacttgaggctgggagttcaagactagcctgggctgcctagcaagaccccatctccagaaaaaaaaaatttaaaaattacaatcacgtccctgtagttcagctgcttgggaggctgaggcaggaggatcacttgagctcaggagttagaggttacagtgagctatgatcgtgccactgtgctccagcctaggtgacacagcaagacgttgtctctggggaaaaaagaaagaaacggaaccacgcggtgtgcagccttctgagtctggcccctttcggtgagcagtgtctaaagttctgtcgcgtgttgcccacgcgtcggtggctcgctccttgcaactgctgagcattgtatggctaggctgtagtttgttttcacttcaccagttgggaaacagagaaaaggcactttttaaaaagtttaaatctgtagaattttggtttttaccagttctcttctaaatcctgagggattacaggaaaagttgttgtatttcagaatattcttagcttgatgtgacctctgtccccgttaaggccctttgccgcaatgggaaggacgtcgctcggtcagaccctgaaggtcagaggggcagtttgggagtgtgtcaacattttaactgtatggactagagccaagagtctcaaggtttataattcccacgtattcaaaaagaaaaaaacaataaagtgagaagtcagtgtagagtgaaataacctgtgttagtggggaagaagtgtttttaaacaggatttccataacgtataacatcaacatgtttagagtggtgatgtttcattgggaaacgaacagtaaaacatgaaagcagggaggttttcattctggcagttggcaactttcacggcagatggagaatttcaaaagcaattgctcaattatcaaacatagccagtgtgagttctgaaataaaggtgctgattgaatgtgcagctttatggtggattttgctattcaggcaagcattttaattttctgcctgttaaattctgttttctttagtttttcatatgtggtttattgtagcttaggaatagataactgagagtatatattacacatacaacattctgatatggcaatatttaaaacaacttgtctgttttagaactagaattaaacataatcatcttcagtattttgcaaataagctcactgccatccagaaacattgtcaatgcatctgttgctccttctagaagacacagtctgtccagcacaaagttacttagtccccagatgtctggagaagaggaggattctgacttggcagccaaacttggaatgtgcaatagagaaatagtacgaagaggggctctcattctcttctgtgattatgtcgtaagtttgaaatgcctgtaaacggggttgagggaggtggggaccaggagaacatcctgtgtagatgacacttgcatggaccctctggaacccagaccgcccggtgtcctgccaagctccatcgaaactaaatctagaatgaatgtttacttctgctgtgacatataattggagaccaggcctggccttccagtcactggattctaagttggactgtgagagtttttgcagctgactcatttatcaaatgcccggctattggctcacgcctacatgatgctgggtatgtttgttaatttgagggaagcaatggaataataataactaatgatttaaaaaacaaagtaagtgcattgactgtagtggggttctgattttaaatttttttaaaaattaataccaggagcagtggcttatgcctaaattccagcaactcgagaggctgaggtaggaagatcacttgagcccaggagtttgagacaagcctgggctatggtgtgagacacccatctctaaaaaaataaaaaataaaaaattatccaagtgtggtggctcgtgcctgtaatcacagctctttgagaagctgagggcggaggatggcttgagcctgggagttcgagaccagcctggcaacacagagaaaccctgcctctaccaaaaaaagaaagagaggaagaaagaaaaattagcctggcgtggtggtgcatgcctgtggtcccagccacctgagagactgagaagggaggattgcttgagcccagaagtttgaggctgcagtgagctgtgactgtgtcactgcactccggcctgggtgacaaggcgagacccctgctctaaaataatttttttaagttaatttgtagaaaaggtgttagatgttctttgtcacattttatgatggattcctgtttaaatgccgttctctttaaagaaaaaaaaataacttgtgggagtttttaaccataaaactagcatcacatatttaccatggagaatttacaaaaaaacaaataaacggaggaaaataaaacctcctgtaatcatactactcagagataacttgctgttagattttggtctagatttaatactttttctatatttatattaaaaatatttaaaacatatgcatttctttgtcacaaacatggtatcttatagatactactgtcacatagcaaaacagtgttaaatattctgaatcagaaaaggaagccgactctccaactgaaagaggtgttatcctagagactttttctggtgatgacaatttattaatagtcactttttgctttactttctctattgaagtagtttttctattttgttctacttttaaggataatataatttataatgctgtttttcacagaaatataagaaaaaagatactaattttataagttaataaagtttgatcatcccaaatccaaaaatctgaaatccaaaatgctccaaattctgaagctttttgagtgctgacattatgttcaaaggaaatgttcattggaaggtttcagattttcggatttagggagctcaacaaataagtataatgcacatatttcaaaacctgaaaaaaatcctaaattcagaatacttctgatcccaaacatttcagataagggttattcaacctgtactgtcagatgatcccaaatgaaaaatattaatcgttaaccaaatatcaaggaattgatcacattttacagtttctgcctaggattatgaatcaagatgaaaaggctctgcatgtttaaaaatatatatttttattttcttataaatcttaaatatctacacttaagatttatttgatatgtgggatccattcatattttggattcaacagttctgtcaaaactgtggcagtgataggggattctttttttcccactgaactatcacaaaattggaaaaagagtaattggagaaccccactggcttagccggcccgaagcccgggagagggcaggcagtgctgtggatggggtcatcccagcgcaacgctgcccctgctacctgcggatctcgctgaggcctgcctttgtcctttgacccttggccatttgttagtgtctctgagagctggactgctgtaccctacttccccagggggcctaacttcacacagcctctgccgcagtgcgtggttggaggtgacggccttggtaaatcgagtttcctacctcctcaattatttgtgctcatacactgtatatttttagtgaggtttatatttgggatgtgttttctccttcttaccctttctggcctttctatggcattaatacctggtctcttcttgtgtacttgaaaatgaatctctcatcatatttttccttagtgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactgtacgtcttcatcctgccgactattgccagttgcagttttccctgccttaaaaatggagtattgaaatttttaactttaatttctgatttgcaaaatagtcatcttttgttcttttccttcttgctgttagccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacaggtattgggaagagaaaccctgatattgatttatattgaaaatttagcaggccaagcaaaacaggtggctggctttttcctccgtaagtatggtcttgacatggtcaccgatagaaacatggaaacatctgcaaacttgccgttactcgtgtgtccgatctgactgtttcttgtatttttttctagtctgcccttactaggatgaactgtacacatcagttcatcctttttaaatgagcatgaggttattttgggttgttaggtgttacaaacacactaatgtgtttttgtctattagagcagcatggcccagttgccaatggaagaactcaacagaatccaggaataccttcagagcagcgggctcgctcagaggtaatgctggaaacacaggtcgtccttgtgttaggacaacccaggatataaaggatatagatttgtacgggaataaattcacaggacaagaaatcgatgtgccttataggtgggtttactgcagaagtgccataatagaaccttcctacttttaaaacaaccagatctcactttctaaagagtaaaggatgaccggcaggatcacgtctgtgacgtgagtggaggcagtttgcactcctggtggctgtttgagaggtagcatttagaatgcctgtattcactgtcctgtgatgagtgggaaaataggttatcaggtttatcttagcaaaatcaaagcatgtcatctaattgctaaacaagagttggcaaatctgagagacattactcaatccttggcatgcaggacttacatctgcatcctgttgccattttatgtcttcaaagcatttaatcatttagttgtgtttgcaaagtctttgagaagcctttgtcagaaatccctacatctcctatgtgagtgtatttccatgactgcagaataagttaaacttttacctttttccttcccttgcggggcggggtggggggcagggattgtgtgtgtgagagggagagagagacagcagagaaggagaatataattatcatgctgtgtactttgagctgaaactgcaaaaaaggaaaaacacacaaaaattattatgcttttcagtctttagagtaccttgtctattatgcttttcagtctttagagtaccttgttgatggtgtttttaaatgggattgggcacaattaggtggacagtttgggatgatttttcagtctgtagggccaagctcttttgtaatttgcattatgaagttgtcactctcatagcagatggcgggagataaactattattactttttgaccctagacttagtcttcagtccagatgagggagattaaaagattataaatatcttgtgccagatgaggtgattttattttgaaatgaccatgaattcctatcagttgtcttactgggatatttgatagtggaatttgtgcatttgagtcttagatgatctgttttacatttattaagaaagcctttattagcttttatactgtgtattgcctgttgcagtgtttgagtataaatgaaatttctggaaaatattaatggagtacaaactgtgatacttaaaagtaaactagggcctgcatttgtatcatgacctgtttgagtattgatgagaagatagctgtgaagaaaaaggtttaaacaagtgtattttcctttaagaagccactaatagtgcatctccttagagtgtatatttctagaatcctagtgtgcagagtttagactaagactaaaaaaaaaaaaaaacaaattatactgtaatttcatttttatttgtattttagacaccaaaggctctattccctgctggacaggtttcgtctctccaccatgcaagactcacttagtccctctcctccagtctcttcccacccgctggacggggatgggcacgtgtcactggaaacagtgagtccggacaaagtaagtgtccagcgtgtctgcatgggaggcacagggcgctgagtgcctctgtcacctgtggcagatacagagagtgcagaggaggtgccgtggacccaaggagttctggcgctcggctcggctcagtgaagctgtggttagagacgtggggggccatcaaggtctgagggagccaagcagtgctgatgtgggacccttttggtaggagtgtggggtgagtagttagtgggtgaatcaaggaatagtcggccgtggcctgcaggcccctgactgcacaggccttcaagcacatgtcaatgccgttagcctccctccatctcctcataccttctggccacctgtgagttgcactgccactgccagccattctggtatgttgtcagcacctccactgctcatacctcatggttagggaccacctggagccttggtagagccttggtagagccttggtactctactttcctggacaaagttcagcttatgaatatgaatttagatttcaaaaaccagcagcccaagtataagaaagcgaaggttcagtcctgccttcttaggctctattcgctaagcacctgccctgccctggttgctggggagagatgagtaaagcagacaacccaggagaggatggcaaaggggccgctaacccttagtggtttagctatatttggaaggcctattggaagttcaccaggtgaagggggaggctgtgagggtgcccaggcaggtaacagaagtccaaaggggaaaacctgtggtgtggtgagccgtatagccacagcctgccggccggcagccctctcagcctagtgcggtgttcccaagcactggcctaggcctgtagctccagggatgtgaagtccccttgaacgccgcccatcatgttccccttatccatttttttcttcccaggactggtacgttcatcttgtcaaatcccagtgttggaccaggtcagattctgcactgctggaaggtgcagagctggtgaatcggattcctgctgaagatatgaatgccttcatgatgaactcggtacggggggagcagtggaggcaaggaatcctcagcttttcttgtgacttccaagtgggatttgtctcatcatcatgtgacccacttgttgacaacacatgttggggactccagtctgggcagggacgggatgtcggagagactccactctgaatggggccgggaagtggggaggactccatttcagatggggtcgggacatgggggttatgctgatcgagacagaaaagcacattgtttcagccacattagaatccacggaggtgttgttttgaaatccagctggccccaaggctgggtgtatggtttgggatgagaactatctggcctccactggaggaacaaacacaggatgttatcatctaagctccatggccaagacagaatggaagtcaaggttgcgtatttgccgtagacttcaacacagtgtcgtaatgcgtgacgtcaataacttgtttctagtgtcttggaagttgatctttagtcgtaaaagagacccttggatgcagcgagatttcctctactcacacctctgttagatgtagtgaggttcttcaccccccaaccccagatgtcagagggcaccctgcgcagagctaggaggccatgcaaagccttggtgtccctgtccctcacccgtgggcaggtcctgtgagcagtgggggggccacctcttgggtatggtgcagccatggcccaagcagggcttcttctcagacctactaggacgggagaaacctcctggtgctttagccctgcgttgatatgcagcaaatgggagggaagtgggcacctgggaggacaaatgcctgtagaggccgggagtgacggcaggtgttcatgaaaagagaccttgtggggagggcaacacaacagtgtgttctgatgtactgaagagctcaactgaaaacaacaggagaattagcccaaaatccatttactaaaattgtttatctttttttttttttttgagacaaagtctcgctgttgtcccccaggctggagtgcaatggcgctatcttggctcactgcaacctccgcctcctgggttcatacgattctcctgcctcagcctcccaaatagctggtattaacaggcatgcaccaccacgcccggctaatttttgtatttttagtagagacgggatttcaccatgttggccaggctggtctcaaactcctgacctcaggtgatccgcccacctcggcctcccaaagtgctgggattataggcctgagccaccacgcccggcctaaaattgtttatcttaagattcatgcagtgaaagctaacttactgagtgataaatttgcttagtgatctgtttattaggttttccaaatttgctaattgggctttgaacagctgtaaaagttctgactgtaaaagaaagcttcaacttttggcattcatgatgcttttctgagtattaaactaagatagatgttttacctgaaggatcggccaccaatctttaaatggctaaacaaaagggttgctaaaacataatccaaattgacataagaaataccatttttccaaccaaaattttggcattcatatggctacttttacgtatttcagctgcatttgaacatctttttcaaactttagggtggttggtgtatcactgaggtcttggatgacactttagctttgattttgtttttatgaattaaaattgtcataccaaaatttttatttcaagcaaatccaagagcataaaaaattaaaatattacttaaaatactaagagagaacagatatatattttactaagcatatgttgaatgaaattgttcaaatatttataacaggcatagagtagaattttcttaaaaatatttttgatggtataccaatttgtattttctcagaaacatttgccttattcttttttctgttgtgtttttcttacctgattgaaagctcataatctgttgttattgtttgttaacctttaatgctctgatttcaggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggtaattaaaattaaaatttatcttatttttaaaaagcattccagggccagtatagtactttgcaccaagtaaatgtacaataaaggcagtggatctaatacattgaaagcgtttacagaggtagctaaagagcagcacgggtgtcctcggctcagaatttcttcctgtgtgtttgccactttgccattcattgacatggtcatggacatagggctctaagcccttgaggaaggctgggccagacctcaggggagatgcagccccaaaccacgtgcagtcctgtggacggatgtgtagatgtgccactgaggaacaatgtcttgagctttcatcagattctcagagaattgcttgactgcctttcgaagttgatgcatctgtgctcacgtttgcacccacccacgaggtccttctgtttcaggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggtaagaggcagctcgggagctcagtgttgctgtggggagggggcatggggctgacactgaagagggtaaagcagttttatttgaaaagcaagatctctgaccagtccagtcacttttccatctcagcctggcagtaagtcttgtcaccgtcaagttattgtagccatccttcaccctcacctcgccactcctcatggtggcctgtgaggtcagccaggtccccttctcatctgcacctaccatgttaggtggatcctaattttagagacatgaaaaataatcatctggaagtactttatgtcttaagttggcctggacatgtcagccaaggaatacttacttggtttgtgttagtgcttgtaattcgcccccagaatgtgtacacgttctggatgcattaaagtctggcctgtatccttaaagggccatcgctgtgctgcctgccctcagcaaggacacactttgcagacccacagaggctccgcctccacctcacaccaaagaaagggaggagtccaaagggcatcagtgccattactcacaaaatgataaatacacccttattctgaaccacgtggagtcatatggtttgtgatccctgtccttcaggtttcagcttagtggggaagtgggaaagtcagcgtgtgatcacagcacagggtgattgctgctgattatattatgtgcctgctgtatgcaggatgaaatactttatatgcgtcatcttatttgactctcacaaccccctgtgagataggctctgttactcccatttgacaggtgaggaaagcaaggcttagagaatttcagtgacttgcccaggtcctctgagctaggaagtagccattctggcatttgaacccaaggcctgctatccctagaacccacgctctcaaattcaacctatgacagaggcaagccctggtgctgtgggagccccaaggaagagcctctggcctggtggccacgtagcccaggagagatttctacaggagcccacagcgctgaaggagagagaggcagcagagtaagggggctttgtggcagagaggggactggcactttggggaataggtgggtcaggactgaatgtaatggagccatgtcagagctgtccttctggaagggcaagggcacctggacgcgctgcccctcagtgctttggacggttccacaactgtgattcacacggcttccccaaacgaaggtacacgagtgggcattctgtgactcggtacttccctttaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccggtgagtccccgtccatgaacggtgggttcctatcatagttcctgtctgcttcaccatgtttttattttgtgctgcctgtttgccaggtactaagctaggaattggggatggagaggtagataaaatatgcatcaggaagggctgggccccatctcttactctccaatatattggagtctacactggaatttaactggaatttgcttttttagtcattttatttagattttgaagtttcagctttcatcaaaaatacctctaaactttatgtctctgtgatctttggtcttagctgttttatgtatttagtcttatatgatcataagattaataacattacattcagaagattatttgttttctgtcagagttaaaatgtttgtttttatactgcattgtaatattaacgtactgtaaaataaaagtggcttgttcttttcaaggaacagtatcctcaacaagggtcattagccacaatttttaaaaaattggacgtcatagtttacatgttagagggcgttttgaagctttgtatttttaaattaaatgttatagagtgatgttttcatgtttcataattgttttcatctgtgcatttgtagccaacttgaaaacaaagatccagggattactacttaaaagccagacttcttggaggttatagtgatgattttgatagtatcttgagccgtctcataataacctcagggtgagagatggccaacaggagacagtcgagggacttagaaatctgaatgaaatctgaagttcaaatcttcagacatataccactaaccaagagattggtacctcagtctagtattgtctgtttgtctaaaattggttctaaggaatctaggctagtctgtctatccctttcaacttttgtgaggctgcacaaatgtaaaatgttgaataaaaagcactgatggaagtgtgtagaaattcttctctttgttctgttgtaattttagttgcagtgcagcctggagagcagcttcttagtccagaaagaaggacaaataccccaaaagccatcagcgaggaggaggaggaagtagatccaaacacacagagtaagtctcaggacccatttttttcttacatgttgttcctccaggacttaaaaatcattcacagagacgtgcaccgcggtgagtgtggactcctggaagcgcaccgtagctccgctgtgtcctgctgctcctccctagctgtcagggaggctgtagtccattgctttgccagctcttttgtttccgagtgaacaccttatccgtacacatgcggctgtctctgaccctacagaccagctgggatgccactgggggagcgctcccttccccccgcacttcccacactctgcagttattctgagatccttgagggcagggaacaggtttgtcttctttgtgttctcagaaattaatgctcggcctctggtcagcaagcaacaaccttttgttgagtgataatgaataaataaatgtttcccacatgagtattcagtaacctcagtgtcaggttcagccatctgttttggtggatatttaaaagaaaattccgcttttcctacagaaaaaaaaaaaaatccaaatcccagtgatttaagccagttatagacttagacatatactacggcttttcatgcactttcctcccaattctagagtaggtattttactaggaaaatggtggcagtgcctgttgggaggaagattctttggccaagtgtcttttgttcttgccagggcccctaggctgctggggtgcttcagcttctttagcccagtgtctggtggggaatggcccctgttgcctgtcccacagaggtgggggtgcctcacctggagcctgtccacacattttacacagcacgcttacctggagcatcaggcatcttttccatgctctgtggctcaggaaacacgccttttcaatcatgagtgcaccagtgcttttgggctttttctccccgcttttgtgcaatcctggttgtggatggagttttcctgtctttagtcttctgcatagtacttttctcttctggttcccggttcaaggttttgtaattagagaatgacccagaagcaatggcattttaatgcacagccaaggacttctctgaatttgtatctcaaacctctgtgggtccttcaggcttcagtttgtgatttcatgatttcttgttgctacctaaggaatatgaaaacacccacctccctactctgcatcttccagccgagtggcacctcaggctgtggatcctgtgcttctgtggtgaggataagaatagtgccaaccgtgtggattgaaatcaatcagttaatccctccatgtaaagcacctggaacggatgacagtcttgttatgaatactcaacaaatgctatcatgatttttagttagatttccattgctttaaaacagttgagacatcttggcggtttgagttagagcaacgggccctgaagtgggttctgtttgggtgaagatgattatgcttattccccatggccctctttaggcaagagtgggaagctttctttgtttttttaatcacctcgataggacgttacttcttaaaggtcatccaataaatattaataggccgggcgcggtggctcacgcctgtaatcccagcactttgggaggccgaggcgggcggatcacgaggtcaggagatcgagaccatcccagctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcaagactccgtctcaaaaaaaaaaaaaaatattaataaagccaactcgttagcgtggggcttaattgcttaagtccaatgagaagtccttctctatcctaggaagttgcccaaactgtagaatctcgtggcctgtgggtaatagccacgtaatacacactcactgcctcaacaaatcatattttagtaggtatgatattctagactcaagacaccattctgtggatcttcccaagggtgtgaagtgtccacagcgtctgccttgggagtttccatgcccaccagaaccatgccccaagcccctcaagcactctgacctaggaaagccagtgaagcaaggatgacaacatggccctttgatactagctgagggacagacacaggtcctgggagaccagagaaagacgaggggcagaggaggtgtcctaaaggaagtctgaggctgaggagccacaggatggcttccagctgtcacaggctgctgctggccttatcacagagagtgggccagagggctgggaaccaaggccagagctcaggttcaggaccattccagcaatcccagcagaaaatggggagaattgtatggtataggcggatatgaaggtagaatctgcaggccttcagtggccaactcagagtctaagtggattccacagttacagcttgagcagctggttgtaggtcatgctttctacactgggcatataggatgtgttttttaaaaagtcctctcttaaccgttgcttgtttagatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgagtctgctcgttccttgcagaagaccaagtacggtgaaaggcaccggtaggccctgggctgggcacacgtgagagggcgggacagaatccccgcagcccagaggctgcctgctgtggttctggtgcccactgtggttctggtgccaggctgctttcctcaggcaccacgtgtggaggtcgctagtagaaatactgggttttctaaaatgaactgaggccctacatccctaagagattagtgttagacctgattctagagcaactagaccactttgcttaatagcagaccagaaaccacaccccctcgagtgagtgagattttcctttggagataattcatgtttttctacacagttttgcagttgtcttcagaattggtttaaagtaggtgttattgccaggcgcagtagctcatgcctgtaatcccagcactttgggaagccaaggtgggcggatcacttgaggtcaggatttcgagaccagcctggccaacatggtgaaaccccatctctactaaaaatataaaaattagccaggtgtggtggtgtacgcctgtaatcccagctactcaggagactgagacaggagaatcgcttgaacccaggaggcgaaggttgcagtaagccgagatcgcgccactgcactctagcctgggcaacagagcaagactccgtctcaaaaaaaaaaaaggtaggtgttattgatcagaacccttgtttcagataacatgaggagcttagcttgaggagagtgagggttgatggagggggactgacttctgcccagtgaaatggcatcatctcccaccagcccgctgaaataagatgatggggcctgttccttagggcctgcagcatcctcaggcaggaaagaaaggccgacctggcagggtgtgagccagcaggtgtaggtcagggagaatggagccaggtcccagggaagaggcttgtggctgcctgagaagggtgcgtgcctgcctgtgtgtgtgtgtgcacgtgtgtgtatgtatgctggagagtctagggaggcttgctccaaggacgcagtattgtttgatcctgagagataaggattctgccgcagggaatgaaggtattccagatggcgggcttattccgaagaagaggccagtgcctggcggtgctggaagcagttgcagaacagggagttgtaggctttcctgggaagagagcagcaggggtgctggagaagcaggccacacttgctgcatggggttgctctcggccccactcttggtgcacagcgagtcactgtgggttcattagcatctggttatgagacagtaactgctcctttggaggggctcgtggagaccatgcaggagggcacggtcttgaggtcatgccgtccagagcacacctgaggataggccaggacgggctgcacgctgtaggtaaaattcctccagcaagctcttcactggcattgaggagttccctgagtgcggtcatctggaaggcagctgtaacaggcactgcagtctctccctgggtgggtaccagagaggagcataggggagcataaccgatttaaagagagggctttcctgtggtgaggtaagagattagctggtcattatcatagagccccctctgcctttgtgcagatgggctgtgggaatcctggggttccgttgggtcctttgtcacctcactgaaggcatgtaagctgagctggccagaccgtgagctgatcctgccacttgaacagcatcaagcctgcctctggattcttctgtgcatggcacttgtctgagcacctcacgcacagagaactggacttcagagtttacagaaataagctgtatggttcattttcatgcctgcttgccaataaacatatctgagctgaacctcattgaacgcctgcctttattctagcacagcacctgctgtttgtgggcgaggggtgctgtctctaactcctgcctgcttctcccagcactccctgagtggggtgtgccagcagcctcaggatgaggacaggaagtgggagggcagagcagatttgggagggccacttgatggggaaggaagtcccaggaagcagttggagctgttttctgggggagaaggtgccagctctgggacagtgttggggtagtgaggagggagcccagtggagagaagtcgggcttcctgcttcctcacagtatgtctgtcctgactcaactcggatgatgtcacttccttttcatcttctcaggtgtggaagcttggatggtcacccaaaccgggaggggattttggcacagcattccctgagatccccgtggagttcctccaggaaaaggaagtctttaaggagttcatctaccgcatcaacacactaggtactcttggggcctctccttcaggtcaccattgtcggacatctaccgggaggaaatccagagcccccagtactgggatcttctcatttgactccagaaaagatttaagcatgataataatacaaacctatgtgaatacattttgcagtgttggcaaaactccttttatactgagaaaatagatcccagttcctgtgttttgtggcttgaatcccagctttgtgtattccgggcttgtttgaagtcaggaaaggttcatgtgtagtggacaacgtgagaccaaattctgccttagattttgcatttaggctaaacagtggcagcacttgtctcagaatgttttcttgtgttcaccagtctgatcctgttgtgtctcagtggtccattttctcatatgggaacaagcagacgggagcagatggagtcaggtttcttggcactcgccttccccagagcctagaggcagcatggggagaaagcaggcttggggctcagacagtcctggtctgcttccagccctcctacctgagcagcgcagggcaagtccgtctaacctctagagaccctcagttttgtcatatgtaaaatgggggtcgtgtctatttcatagaattgttgcagatttagaaattacatttctaaacaaatgttaccccttatttctaaataagtgtctaaatgaataagtcaccacttttgcccctatttgatggcaagaggtgtgatcttgtggtgggactgtaatcagtcagttctcagtgactgtgccctgctgtggtgtttcctggaatgttcctgtcttgtcctagaaagtctggcaggggcaccctgactccactgtccagtcctctccccagtccctcgggcttctgcagatttgaggcttgtttggatcccagaaggttgtggcaggagacaccttgcctctactttcccctttataattcaatgtccaaagagagccctgagcaggtacctcacgccagctgcctcacggagctcctcctcttcctggctgtgaggatcggtatcagtggcctcctgctctctcccccttgcctaacacgagcacctttgcttacttgggtgcccttgctcttgaactgcccatcggacgtgcgtgacccaagactgtgccgcagtccttgccttgtctgtgctcattttctttgttcatttttttccctgtaacgtaaattgttatatttgtctgtatctgtgtctgaatcagtcctgcacgctctccttctctctgtctcttgttctttctttaccccgtttatcacggggaccccgatgtccattgctctagttctcctgtcctaagcaccccatcccgtctctctggccttaccacaagtggcgtggctgcctcagacatcatgatggggacatgaagcacagctgtcagaaacaactgttcgttagatacactcgaatgcagctcatcaatagggatggagggtctgtcggatgtattttcactgaatccccgttcctaccttgatacactctttttaatctattcttctagacaggtcagaggaaccattactttgacttttaaatttttagcagctttattgaggtagaattcacatactacagatttcacccactctaagcggacagcttggtggccattagttttatccacagagttgtgcagccagctgcacagtctcagggctggactccagggaagattttagcccatttagtgagtggggcagaagtggccctggccctgcacgaggttgcctgcatgggcgtccctgccctgtccctgtgtctgctccactgggggttgaccaggctgccagggccgacttgggcctgtgccacctgcctctcatgtgtctcggacagtgcagccgatgtctatacttcggtttcctcaatgatgaaatggaggggatagtgttccccgcatcatagaactgtgtgaggtttaagggactcactgcccttggcgtggagccttctccaggggccgtgctgtgtcggcgtagctgtcagctctccgttacaggcttgagaagggttgacactctctcatgtaacatttatatttctaggctggaccagtcgtactcagtttgaagaaacttgggccaccctccttggtgtcctggtgacgcagcccctcgtgatggagcaggaggagagcccaccagaagtaaggccacaccctgtgctggttggcacatgggcagttatggccgcttgcaggcctttggtggggaataaaataaggcagcaagctggtgttctttttttctcttaccttatttttgaaagagtagctgaatggtgtcttgactgatattccagagcagggacaaagcctgctgaggtctgggggctgcgattaccaatggctggaatgcattttattacggtgcattccatgttaaggatcaatacgattgtgccctttctggaaaatatcttttagtttatcaatattcagaggagtgtaggttgaattaaaatgaaaaggcactttataaaggccatgagtagtacctggtttcatttttctaatgtcttgcagagattttatcaggcttcttgaagtgttcacgtacattacgctaacacgatattaataataactgtgctctggtacagcggagccagcagaatgggaagttgtggaatgcaggcccttgattctgatagaaggtgtggtttgaactcacagaaatgacagtttggagggtagacatatgtcacaagtcatcaagattgtctttaaattcatgcatagaagctaacagggtgtcataagcaaggcctgtaaaatgtatgagggaattcaaagataatttattaaaaagtaattcatgtttggagttttgtgcccaaaggagtccttgatttgaaaaatgggcttttgcccatcagattgtttcagggcccgtgtgtgcggaggccctgccttgtgccccgtgagctcagcctgacagaaatcctttggtagcacttaaggctcctcttcctcccattgaggcagggaagactctgggttctgcaggcagaggtggttgtgggtgtcttgctgctcttgttgacatgtgggctctccttccaggaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgcttgagttcccacgtgtctctgggacatagcaggtgctggggacagtgggttccccgctgaagcgtccagcagcttcaaccaggccgttttccttcattgctagaattgaaaacaccgtccgtgtggcctgtgcaggagatgcagacccaaaggtggcctcctggtcagtgagaagctggaaacgtgacaggaactgacgtggggttattgagcatttaggggaagacgttagcagagcaggaatgagcaggcaactagtagaacacccacttaagggctcacggacaggtgctcacttaggaagtgagtttcatttggtattacaccaggttcctttaggcaaagcggagggaaagttctggtgtttttcacttgtaagattttgaaggaaacaaaacactctttaccttttttctaaaatgtaggtttgggaggaagctgagcattatcagagggattgtggagcaagagattcaagcaatggtttcaaagagagagaatattgccacccatcatttatatcaggcatgggatcctgtcccttctctgtctccggctactacaggtacctgagggaaagggtgcgggggagcggttgtacttgggctagaatgagagaagactggcatgctcaccacaccagtgatgcgggaagacctgagtgtggtctgagttggaggctgtggtgctaaatacgctgcccctttcataagcaggagtcttagtcaggcccagggaggaagtaaaatctggaaatgaatgagaagcattctctcctgccagtcaagaaatgagaagcgaaagaattctcacgggctgtaagaccagcaggatttaaaagttgaattagttgcttatgttaagaactcaaccaagttcatctacacaagctgaatctccagcttttcctaagaaaccatgtgtggcagtggctgcagggcagggcacagctgggcctgagcaccccgctccctgcacctctcccctccctgggccctgcctgtcactgcccactctcccaccaagccttccggttgtgtgcctgccctatcacaggcatcggagcttgtcacctggtttaaaagaagagagttgtgtggggatttgggatgcacgtttttcactcaaaagtattttagcgtagagctctgtgattccgtagctatttaggagtttaagcaccttgaaggctttaattgcagaaagttctatgtggacgtgcaatgtgttatacgcagtgtctatgagactcaaatgtttattagggcgttgaagtaaactgagcacttggagggccatggatccagccttcaaggagctcataagtcaggaggacccaggagcaatgacctgtcatagaaggcagaaaagaggggcacagaggtgggtgggaggcatacacaggcagctcctggagctccaaggggagcaagtgcttccagggaagggggcgtggaggcccctttggaggaggcaagttgatctggggtctggcagagggttagctggggacatttagcgggaggctggtgcccgggaattggggggatgcccagcagaaagacatgaggaggctggcctggggcgtgggggggtgtgaaaggttaagtgggggcattatcctgctcccgctcctgccggctgtatctggtcagcctgggcaccgaggtggggttctggaaggcactgttcaccaaaatgcttatctgggtcccccagagagcttgcctgcctggactgtcggctcgcctgcaactgctgactcctaagcttttgcagctcagcccacaaccagttcctattcacagaggtgggagctgaggggtgacaagtgactgctgcagtcttatttgtcatagagaaaaagtgacagagtccagcttgcccactggccctgccagcttaactggttataaagtgacaaatccccaagacccacagggctctgcacaacctgggccctcctgccagtggcggcgagggcaggtggctcacggctgggtgcctgtctgggcaggagctgggctggtatggggtgggcctgcggccctgcccccctgtgcagatcaagactcagggtgctggtgttcacaggtgccctcatcagccacgagaagctgctgctacagatcaaccccgagcgggagctggggagcatgagctacaaactcggccaggtcagtctcgcgcccccgccgcctggcctctgtccgtttctgtcctcagactttggcgcttgacacacccaggagaaaagctcagtgcactttttaaatgaaaggaagttttccttttttttaaaaaaaaatttaatgttcattgtttttatctgttttattcctaggtcccgcaagcagaggaagcattagttttgtttttatttatgttctgtattccagaaagtagttaagagacctcacatgtagcgatagagatgtgtgtaagagacagtgagagggcgtgacttggacttaagcaaggaccgtgagacacaaaaaggggggtgaggacagagtggagtcagctgaaatgctcaggaggaagtagacgccatgaagggccatggtatggggggccgcaggcgtggccgtgagtgtccctggggccagctcttggggggctccctgagtgtccctgtccctgtggccagttctgggtgggagccccgtgtgcaggcagacagctcggccacttcctagcaggtcacattggtctgtgcttctgtttcctcctcagataagtgaagggattcaagggtctgggtgtggtggctaacacctgtaatctataacattttaggaggctgaggcaggaggcttacctgagctcaggaggttgaggctgcagtgagccatgattgcaccactgcactccagcctgggcaacagaccagtactctgtcccttaaaaaaaaatgtaaacagaaacgtagggccatttgcatatgatggcacatggcgtggagccctacaggtgtatgctgggcggggcccggctgtgctggccgacttgcacctttccctccaccccggtgctgtgtctttcgctcaccgggttcctgatttagtgaaagcagttgtgcaggacagttctctttgtagcttttgtttctgtggaaatgggtcagaatatggtgtttagaaacacttatgagctctgagagtttcctcttctgagttcctggcctgcagccttcacagcagaaaccctgtgatgtcacaagcctgtttctgttccctgctctctgcctgtactgtcctgttttgtgcctgccggtttcagtgacaggaagcagggagctactggaccagcctgtatttttctagacatagttggaaaaagaagtcccactcttctgtcctttcacctttgacagatgtttccaccccaagataagtgaaaatgaccaataggatgcactgtatttttcatgaaagtgtttctgaagggcaggctgagagtgagaggcctggggctcactgggtgcctctggccttgtcctgggcccagggacactggtctgtgcccgaggtattccctatccccccaaccccgctgcatttggccacatccttcaatgtttgcgttgtgtccagcgtccgcaaaccaactgtcatgggatcatactggggctgaagtacggtcccacccctgccctgtctggggctgaagtacagtgccacccctgccctgtctggggctgaaggacagtgccacccctgccctgtctggggctgaagtacagtgccacccctgccctgtctggggctgaaggacagtgccaccccttccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctgggatgtttagcccctagatgccactggactgagccgctacttgcttttgggaaagaggggtgggggttaggggtctgggcgaggggagtgcaggggctcctccttggcctgagagctgttcatacagactcctcgcccactccctgcagggtgctgggtcccaggggggaaatggcccttggtgccaagaacgtgagttggggctagtgccagtgatgatggagaacagctttttatgggcacacagcccacagcactgtgccaagtgctcgaggcttcccgagaaccaggcagaaaggaggacagtcgaggtgtgctgactgcgtggtggctgcgtgatctagagcgcgggtcacaaaggcgcgagggagctctggccttgggtttaccgcaatgactgccagtgcgggagactggaaaaggaatctcacgtattggttccgtgttttggggactccattcagatgtcacttaggagtgaaagcatcccttcgtagagcctctttctgtgtcaccctcctcagctgctcctggggttgactggcccctgattcatgcctttagcatgtgctggagcttcccagcagctgtccagcccctgccccaccctctctgtgggctcccttgcccgtaacctggggtgtctgaacgacccttgctaaggggcagactgttagacggtaggcatgtgctgagtcccagtggccacacccacccaccaggagcctggcactgtggccgcagcactgagcagtgccccgtttctgtggcaggtgtccatacactccgtgtggctggggaacagcatcacacccctgagggaggaggaatgggacgaggaagaggaggaggaggccgacgcccctgcaccttcgtcaccacccacgtctccagtcaactccaggttttccaatggcctttttctttttaacagaaatttgaaatttcttatcagtcatttgatttgtttgaggtgcttcttgaaatgagcctctcatctcatgtacttggaaaatacccatctcgcatattccacaggaaacaccgggctggagttgacatccactcctgttcgcagtttttgcttgagttgtacagccgctggatcctgccgtccagctcagccaggaggaccccggccatcctgatcagtgaggtggtcagatccgtaagtgagccttcccattcccctcacacctgcacgtgccacacgcaccacacacgccacacaccccacacacacacaccgcccacacacatgccacttgcacacacacccctcatgcatgcaacacacacacaggccacacgcaccatagacaccacacacacatgccacatgcacacacatacacggcatgcaccatacacacaacacacacagcacacatgccacacacacacgccacaccacatgcaccacacacatgccacatgcacacacactccacatgcatgcaccacacacacacacacacaccacacacaccacatgcaccacaccacacaggttacatgcacacaacacacacatgccacgtgcacacaccccacacaccacatgtatgtgccacacacagcacacaaccacacacatgcaccacacacatgccacatgtgcatgcaccagacacatggcacacactacacacacgccacgtgcacacaccccacacacatgtacgcaccacacacatgccacacacacatgcaccacacacatgccacatgtacacacatgtatatacacaccccacaccacacacacaccacttgcacaccacgcacacacaccacatgcgcacacacacaccacatacgccacatgtacacaccatacacacaccatacatgcaccacgtgtaccacgcacccacacagacacagcacacgcatacaccacacacacacgcacacatgcgtcccgcacagtaatgtctcttgggtgtaagaacacgacttgccagtagtagcgttctggatgcgttgcctggattctaacagcgcgattctccccttgccctcctggttttccacatctccagcttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggtaagtgacaggtggcacagaggtttctgtgctgaagccacgggggcccatctgccttgggacctggtgttggccagaggtgccgggtgcggctgcctccttccaagagttgacccgaaccggactccacggcccacgtgagctgcagtgcttctcagatggagggggttcagcgacggtcagtgccattcacaggtcactgtgatgtgggttgtggcggccaagccatggtttggggtcccgtatccctgggcttatgacatcattgtagtagcccatccccacagaaccacggtgtgtggtggcgctgaggcatcgtagatggtggaaatgctactggcttccccatgctctgccctgaggcctgactgcctcactccccttctcagttatgttccaggccccccgagcttcctggctggacagcttctctcctgggggccgttttgtcacagtgaccctgtgtttctagtcccaaatctgggtgctatagtctctttttagcgtggtggttgtcttagtcttttttggctgctaccacaagttaccttagactgggtaatttataaacagtggaaatttacttctcaccgttctgggggctggaagttttcatggtcaaggtgccagcagatttggtgtgtgatgagggctgctctctgcttcatagatggcatcttctggctgggtcctcacggtggaaggagtgaacaagctccctcaggccttttagaagggccccaatccacaagggctctcccatcatgacctcatcacctcccaaggccccaccttcttgtactgtggcactgcaaattaggtgtcagtgtaggagtttcaggagggatagaaacattcagaccatcccagcggtcaagtgttcatcctcttgagttcctccttattctgcttctggtttatcaggattcagccagtgcagcatggtacctgtattctgtggcacatcaccacatggtatttgccaagtatccatcacctgcacacgtgaaatcattgcccgtgggtcccgacatctggcgaagcatattcaaggatggcagaactgtcagagctggcacctctggttccttgtcatgtggcattacctagtaatccattttatgatagcaatggaaactcatttcttcaacaaacacctgagtggctgccgtgtgccagccgtctggggcccttggtgagaatggcatggtggtgcccatcagggcctgcctagcccgtgctctggacgggctcctgtgtgtcaggaacgacaatgctgtcatgacggtgaatgatttttttttttgccatcactccagccgctaacatttgcggagctcttcctcccgcacccccacctgacaaggccaagggtgaccttggccccaccctaggcggccaaggtcagaggttagctggcttgtctgggtcacacaaaatgcagcagaggttgaggtgagcacatgtccgtgacctggagcctgactccctctctgcgagtcttgactgctcttgcctagactctgtcctccccgagcccaaacgccagtcatcttcccttgtgggtgtccttcagcctggtgccatgctggtgactcagcagccgtccagggagtggaaacaattgagtgtgtgggttccctgtgtgggcatctctcttcacggcgaacaccctctgggtgttgcccacacgatgtcaaagcggctcttggaaggggtccttctcctttgtgggaagtttcagctgctgggctaacttgaattgtaactgtggttttgtgctcaggcccagatccccctaggcaagtgttgtgccatcagtaatcaaatgagaaataatcattttgaaaagcagatcctaaggcaggatggtcatggacactcactcccagctctttgtgcactcatgctttctggaagatggccatcctctgtgaaggttttcagcgcgtcatgcttggtacccacgtatccagagcatgtcgttttgaggtatttgcccaccgttgtgaaatccgtgccacccgagagcaggtcctgatgtggggctttcagaagtgggacctggggccgtacgcagtccttagggaggggccgtgtggcgttgtgcgtgtgaggggatagcacagggtgaggtgggggcccaagaaggaagtgacccacaaagaacagcctcctcttttggtccttgttcctgggatggctgggagtggcttctgtgtcgtccggccatttcccctgcggagaggctcctaccactgccgagaacctcatcattccacaaaaacaagaggccgcctggccatccagcgctccatgggaattctgtgtccccatagtcttgggctgaaggagggtgacattccttgctgacttctgcaggggtctcctcactgttaaagagcagattgaaagtgaagaacgtgggctaagtgtttaggtcgatatttaaccctgctaggttttggatactaagtgaaattgaggccattttggttgaagttgacagaaaccactatcagggatccccaagactaccccaggcttttctagaaagactctcagctaagatgtgttatggtaaaagcacacaaaacaaaatcagcaaagaaaattagcaagggcagaggcccatggggcgatgtcccgaggacaccaggcttgagcttccagaatcctctcccagcggggtcgtgcaggacgcacttaactccccgcacagtgagccgtgacagcgcgtgtgcagtgtcgtcgccaggaaagcacactagagactcggtgccagggtttttactgggggctgggcacatgggcaccctctgcctgcctcgtgcccagactctggactcccggagggaaggcaagttctcagcaccaaccctggtgcccacacaagcagctgagcacagggagcccctcctcagtgaggatggtgggcaccgtcccaacaccagccaggggccagccttgcacacaggcctctcaggatggtctccggcctgctgtgtagtctcttctgcacacaagcgtgagggcagcgcccccgcctcggctgtggggaggagccactgggacgtgagctctggtggcatgcagcagcttttgtctgtgtgtgcctaggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccagtgagtgggagcctggctggggctggggcgggggtctcagaatgagctgtgaaggaagcagcatcaccctctccaagtgcccaggctcctggccagatggcaggccaggtatcagtgggaacccaggtgggtgccatggctgaggtcagtgagacgcaagagcacaggtgcgtcctagaggcttcctcgggcacctccagcgagctggagctctcgcctctgctgctgtctcatgtggcgcttagcacactctcccacgtgcccattcctgactctgctctcgaggccatcggctctcattctctgctcccagaaccctgttattacccaggctagcctcctctctgcaccttccccgccctggcccagtacctccctcttgtttccactgtgattccgacctcaccttatcttaaagctgctggacggcaggttctgtacacacgtgtccttgacaaagcacggctggtgccgcaacccctcagcgagcaagtcaagctcttcacagcgatgtcttacaagcgcagagggctctgtgacaccctggtctcaccgccactcttccaaagtcgcagaggctttagcagagatgggcccagcctctctgagtcataggcttctgcacacgggagctgtctttagagggagggtggaatttcatcagccacccacatgggggagttgagggcaagaattaggagcaaagatgggaaggggtctgggaggaatggccagtgatcccctttgacaagtgggcaggaaacgggggctaggtcaaagttgagtggaagacctggagggagacgggaaggtctctgtaggcacagttcagacaggagggaggtgtgagccagggcacatgccggtggccgtctggcaggatttgggacatgctggagcagggacagcggctcatcaggggccattgccctcatccaggccagagtgtcacaagcccgtggggaggcccttctcgcctgtcatccttgctgggcagtgggtgctgtgctagcaggacaggcggacggctggcaactgtctctgcatccctggagcctggcatagggccaagtcacacggggcacaggcctgcaaatcaggcacatatgttggtgcagtgacgtgattttggggggcagccccagaacaggccccagacacaggccaaagccctgcctgtgctggtgtgttgggctgttctatggctcttgctgtgggcatggaggactcagggaaggagagttgaggtggtccaggagttgcgtttgggatgcagagagcttgtggcatccaggtagaaatggtgcgtggggctgacctcagcaccatgggcagaggggccgtgtcacgtgcctccgaggtggaggtgggaccacgtggtgacagatatacgcatcactgggcacgtttttgtgggtgttggggggcatcgtattggctcctctgttcacagtggccactcattcagtccctggctaccaggtcctcactgtgccatggggaaggccggcgctgtcgggggatcacagaaggcagcacgtcatgatggcatgtgccatgaaggaaaagcacagggcactcaggaagtagaggggactggcctggggtgtgggaatctagggcctcgttgagggacagagagaggaagtgtgtggtggccagcatggaggtggccacaggggaggctgagttaggccgagagggcagggcgttggggaggtagacgggctcagccactcagggagtggtcaagcagaggctgaagggtcaggccaggttgcaggggcctgggggagccactcagggtaggcgctcccgggagcccgcctggcccatagctctacactcccgcgtggggccggacatgctgtgaagccctctccacgttggatgggggtggctgagcctggatgctgtctcccgttttcagctgcgtgaacattcacagccagcagcacgtactggtcatgtgtgccactgcgttttacctcattgagaactatcctctggacgtagggccggaattttcagcatcaataatacaggtgagtgggccctggctgtcttcctctgcacacggggagtgggcttcccttctcttttccttgcaggatcataccagtgggccagttttgacttggtcgggaggaggcatgaacacctgagactgtgcagcgattctttgacacagaggcctttctccctgtgcagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggtgagcatgtacacggtgcccataaggccagcccaagtcctgttcaagggaggcaggagcatgctcactcaagggacctcgactaggtgccctctgatttcacacttctggtgttgccccaagccggccccatcaccttgcaagaaaggctctggagcccccagggctggagtacctggtcagggttgaccgtccctgtggtcactcatcccatgtggctgagctgggctgggtcctgggcaagcaaggggctgatatcacctgctttcagatctccagggactcactggacccctgtgtacaaagcactgtctacagagcctattgggttgtatagaggtaaccttcgtactgaacacttttgttacaggaaaggagaaagtcagtccgggtagaacttcagaccctaatcctgcagcccccgacagcgagtcagtgattgttgctatggagcgggtatctgttctttttgataggtaagaagcgaagccccatccctcagccgttagcttccctagaactttggcctgaagctgtgcttttgtgtgtgtctgctgatcccctggcgctgttgctggagtcctgccagtgattccccaccacagcctgaccatgggctgccttggctcagggttccactggcgagctggtggtccttggaccccagcactcaggtgtagcgttgaccagttccaaggttgtcccagtgcctgcccatctctcctgagggctcagggacagtacctggcagttgggggtgtggcagggggcaggaatgaccagcctctgggagggtggggcagaagcctgtacagtgaggaggagctggctcagcctggctgcctatcgtgagaggggagcccacggggctgtgggaggggggccgtggtgcctgtgagcagggtgaggagcagcggcaggaggatgaaggtggaacccacacatgcatctttgagacccgtgtggtcagtggcttctgccccccaccaccccccactgctgtgcgtgcatagaattggcttccctcacctgctctggaagtgggttaggagcttggtagggctttttctcaaggacaagggcccctgatttgctctcaggcctcagtcctggcgacatggtggatctggagccttgttgcactgccttgcctgtgctctccaatcagggtggccagtggggagccatttggcttttctcaagagcatactcaggtggaccttgctccactgtttgaccagatgaggcattctgaacagccaagcctgtgctggtctgttttcatgttgatttttttttttcttttctttttgagatggagtttttcccttgtcacccaggctggagtgcaatggtgtgatctcggctcactgcaacctccgcctcccgggttcaagtgattctcctgcctcagcctccctagtagctgggattacaggcacacaccaccatgcccagctaatttttgtgtttttagtagagacggggtttcaccgtgttggctgggctggtctcgaactcctgaactcaagtgatccaccctccttggcctcccaaagtgctgggattgcaggcgtgagccactgcgcccggcccccatgtcgatttttaaatgcacctctgcatcgttcttcagtccccatatgctcactgagcaccactgcgactggcagacgggcacagggaggcgccacgaccagtcctggccttcaaggggcttgtggtctagtgggcccaatgctaggtggcgagtgctccaaagagtgtggtgcacgccttccgcttgaccgctctccagacgccacagggaggcacctcgcagctgaccacagatttctctctgtggagcagtgtcttcagagcggctgccatgccactgctgggcgagggtctgcgggcgggtagagccaggagcacctgtgaggaagtgcactgccattttcgtagctgcttcccgtgtgtctcagttacacacggctggcatgtgtgcactgatgagacgggaacgtgatggttgcttttcagcactgaaagggatactgctcagggggcgtgtttcaggatctggttagggaagaagcagcgagagcacagatggggccctgtgtggtaacaagaaaaaagtcctggttgacaacagtgccacgaagcgttagaacacatagggatgtttgtggagcatttgcatgtggaaagcagcaaaaacataatgggaacgggttcttttgttatgatttttaaaaatctcttttgtaacatccttcccgctgcgccgtttctgcatattcctttatgtagctttcaaactcctcttaggagttctggtccctacagggcgtgggagcccaggctttacgtagctttcaaactcctcttaggagttctggtccctacagggtgtgggagcccagggcctgtgccgagcagcctgcctccacgagctagacagaggaagggctggggttttgcctttttagtctcaaaattcgtactccagttgcttaggctctgactttccccacttggaaagtccctcacggccgagggtccctcccagccctgatttcacatcggcattttccccagtattagagccaaggccctccgcgggcaggtggggcagctgtgggagctggtgccagtctctgacctgcgtccctcctcccaggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgaggttgcatgtgggatggggatggagtgggaaagcctggaggtggagttgcctccgacttcccagcagattcgccagcagagcccagctcctccgctttaaagcagcaatgcctctggcccccaccccacccccgccacccaggcgcagcaggtgcttcccgtccccccagccctgacactcaggcacctgcttgctccttgcaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatgtatcctctctgggtccctggtgctggccccgtttcccttgtcaacaccgaggctcatgtttcatgataaggttttgaaacctaacctttgcaaaaaccccacagatgccagggtgacaggccctcagccccagggaagtaaaatgctgacaggggtacagaaaggagcacgtccagacatttgctgaccagggcctctcagaggggccggtgtatggcaggagggtcgcagctgaggggcctttctgtggagggcctgggtgaggggagcgagggtgggcggtggtctctgcagacgtcccgcccactcgcgggctctgtgtggctgggcttctcctgacactgcttctcattagctttggtcattgtgcctcgatcgccctctcggggaaaggcttaagtaaagatccagttcccacccccagatgctggctgccaggagtttccctttccacagcccttccccaagacagaccacaagagcctccaagcagcacagttgtcctggtgctgacagcacagccttgcccggcgtgcctggcacggctctgccctcactgcattggagcagggctagtggaggccagcggaagcaccggccaccagcgctgcacaggagccaggccaggtgagtgctgccgagtgggtgccctgcctgcagggcatccagccagccaagggttgcaggaatggaggtggaggcgctgatgcagctggaggcatccaggtggcccttccggggctctgctcgctctccaggctccctggacccctttgtagactgtttcaggagaggaactcccaggtgaggacagggaggcagcattcccctcatttgccggcctttttccttaactcctgcaccagcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatcagttgttgctgtgtgaggcttgctttgcttcatgagaacctagaccttgctgagctggagtcttaggaagcagtctcctaagtgcttctccagcaggggcagaaactgtcccaccagctaacatctggcattatggagggtcccccaggcagctgccagcagggacaggccccgtgttttctgtagccagggatgaggaagtggccccagggcatgggcctggctgggtgcttctgcaagggccttcccaaaccacagtacaggtggtcttcctgccctgcagatgggagctgtgggagctgctggagctgctggagccttcatggtcaagtgacatcataagcttatatgacatacacaagcctcaggacttggcccatggcactgaagcaggtcatcaggcccagcacagagactagagctgtgttctcacagggcccaccacccttccacctccttggccattgacacctgcgtccctggcccagctgctcccaggtaacccccaaagcagctggcacatcccacctctggtgtggccggggctgctgtgtgtccgcagggcctgccccgtctattctagcttgtttgtcctgtctgaaccagcgcctactccaagaagcctctgctcagcccagcggggatgcttctaagctccggacgagcctctcggaagccttggtgattggtggtgtagtcatcttgggatgcagatgtcttaccaacctgcaagaacaaaaaccctgtggcttcctctggtgcagggtatttagtcaatgtttgctgaggtcccgtctggttctggctaattggcaggggtcgtccacccattctttccctgctctgctgtctgtgccaggagagacgggggccagtcggccaaggggccagctcctgctgcctgctcctcttgggcacgtgcgggggccccctttctctgagcagggatagggatcagtctgccggagggatgtggtggacaggcctaaagcatttggggcggggcatgccacttgagctccctaaatctgtctcctcataggtgacaccgctccagggccccccagtggcctctcctttcagagctacctaaattctggtcacttcagagaaatggagcacccccttctccctggtccaggtgtggacagcctggcacactgagcacacctggcatggctggtaatttcagaaagaagaggggccggggtccagtgggaagcagcggtgaacccctcgtgagtgggctttgcagtccctccccatgccacggcagagctgccctcaacacagccttcctcttcctcatcggagagcacaccctgtccccttgccgagctgtgccctgtgccttcggtggtatttgattttggctgctactggctttgttgggatctggaagtcgcttcccctgcgtggtgcgtggagcactgtaagtcagatgagggaagtagccagggtgaggtgagtaccgggtggagccgccactgaagggactgggtaggggggccttgcctctacatgatgtgacacagccaaccgaggacagaggaagccccgttcctgggggtgtggggtgcacccctcagggaagcctgcagtggggcctgaggaaaggcatcctccgcgagcccacgagtctggtccatgagcaccgtgacagtgtctgtgggtagaggtggacccggccttgtgtcatcaccaggacctcttttgggaaaccatgtggacatcgcttgcgggtcccccaggctctgcagccccagcagcctggctgccttttgggcaagtggcttgagccacagaggacccagtcctgttgcagccacatcctctgggggggcccgccagtgtggccggctttctccaccctacaccaggcctccaggtgtcctggtcgggggtgtctgggccctgggtgggccctgtggacctgtgaggtcagggtcagggcatcactggaggcagagggctgaagttgtgggtctgggttccccttgtgtgcacaggcccctgccctccatgcttggtcaggcagctacccccaaaactgctaggacaggctggtcctgaggtggatcctggcccctgtaccctctggacagcccacccgcccaaccttctaccctgccccagcggcggcagtgttggccacatccttcccctcctggccccaattgctctggggaagtccaggctccggagcctgcccaggggccccccgtgatttgggcccaggactccacgtggttctctgccttcacccaagccctgaactcctcagctgccaaatccccacccatctgcacaggctgtgctcaccactgctgctcctggaaggtgcccctcagtgggacgcccacctcctctctgggcttctgtgtttgggagccctgctgcccccacccttggtcagtccccatgtcctgctggcctgtcaggcagggcagaaaatccacccagaaatgctgagcaggatgagagtctagttgggcccagcctcattatttagaagggatggaggcctagggagcatgcttctagcctgagcccagcagggccccgcccatgtcccaggtctgcaccagggacagctcctgccgaggcctgacctgccccttctccctcaggtgctgctggttgaccagcctctggccctaggagaccccgtagcgactgagggtcccagcaggccatgcagctttgccaaggtacgagcccctccccagcaggggacagatgtggggaccctcccaggcaggagcagctgggtgcctggtgctgccatctgctgcctgcctggttcttgtcctcacattggaggtcagtgtgagggctctgcctcgggaaaggccatggagcttgccctgtccagggcctcccatgtgcactgagcctgggaagagagggttggagttgagccttttaccctgggaatgctgcctggaggatggtgcgggtgtggggtggcaccctgccaggcagggccctgcctccctgcgcccactggaactcgggcaggcaggggtgtaggtgcctcctctagagccgtccggtgggggcccccggcagtggtggtggtgtccactggccagcagctgccccttcagccaggacagtaggcctgacgctgtccccagcagctccaaggtggatttgtggaagggggtagagggcacgtagaggccccatgacctccccagggttctgggagggctgtgcccccttagccagcaccatgctgggtgatatagtcagatcctgttacccctgttgtggaggtgaggaaacaggttagtggggaggacatgactaaggtccatgctgagtcgctagagctgcacccagaaccactgctgggaccccatgcctttctgcttaccccttgtgccgggagatgccaagagatgctgggagccagccccacctctgcccttggagtcatggctacggaaagggcattcggaccggtccctgacctcaccggggagggccgaaccctgttcctgaggagccagggcttcctagaggaggtaggccttctagtcactccttcatctgcaggcactccacagagctctctgtgccagcccccagcacggagggctgaccttagtcgagtggagatgccccagtgccaggcagtagggatgatgtctcctgaggcccagatggaagggactggactagtctcatggggctgatggtggggccaggccttgaccagggacccagtgtagggggtgcagagacccctctgagttcctcacacatccctggggccctccccatacacttcctatcctgactgcgggcaagagggagccccagttcgccttccctatgctgggcacccacagtggggctgggcacccccgccatgcccctgccctgtccttcccctgagagcctcggtcccacctccaaggtgcctcagaggacagcaggggcagcgggcagaggccgagatgcctcctcattccaggctcagctgcccttcttggggcagcccacacctgagagtctcctgcagttggtcaggcctgaggagggcaggggggtgcctgctgtccctctgctgaccacagtggcatttagcctgggcaccgcgcccagcacagtccatgctgcacaggtgccgtgggctccacagagccctgcctgacatgcatgtgttacgtttcgggtgccgatgcccttgggcggcacttctccgggcagaacccccaggccaccgctccggttccggttccgctgcatctggggctctcggcaggctgtggtcctccggccagcctgggggcatctcagtccctcagccccacaggggcctgccccgcagcctgggcctcgagccccgtctccgcacgctgtgccgaatctggctgcccatcagctccctgcgtacccagactgtgccctgccatgcccgtggctcttcccaggagtgccctgtggcctccccctggcttgctgggctgattccctcctgtgtctcaaacagagctcacctttgccatcactgctgtcctcaccggccggtgccagaggcccgtgtctgtgtaccctgtgtctgcacctctgggcagggcctggctctgaccaacccgggcttccagtgtccacagacctaaggcccagggcgcctgggggctggagcaagagaagcaaaaggagccaagggtgggggtttggggttcttgtgagggcccagccccaggaccccaggaccaggacacccaggagccccagggcccagccccagttcagaaggcaggggccttctgagggagcttaagggtcccacagcccaggacccccaccagggccagtggccagcgttgggggactcagcctcctcgtcgctcgtcctctctgtttctcccaccttttgccccctttctccttgcctgttcccacccgaggccccctcttggcctgcgtgagccggggcggcactgaactgggggccgatccgcctgggcggcggtgagaggcagggccgggagccgggccgctgggtttgggcctggcccgctcgccgcaatattgatggcccgtcagtgcagccctgattcctgtgctttcagttaaaaggtttctgttgttgtagcttatgcagttgctctgttgctatggaaacgtgacatcaaaatgacgtttcccgtttaaaagcttttaactaaattcctgcctgtcagatgtaggccccattttgagcgtggagctgccttcgagcgagcgtgagcggcgcctcccgcccatggtgcgtggggccgggccggggccctcgctgagcgcgctctctcaccccacaggcgcctccggcatggcggcggccgaggggcccggctacctcgtgtctccccaggcggagaagcaccggcgggcccgcaactggacggacgccgagatgcgcggcctcatgctggtctgggaggagttcttcgacgagctcaagcagaccaagcgcaacgccaaggtgtacgagaagatggccagcaagctcttcgagatgaccggcgagcgcaggctgggcgaggagatcaagatcaagatcaccaacatgaccttccagtacaggtgggcgagcgggcagtgtgggccccaccaggacgggcgggcccgggcgtggcgggccgctcctgactttcttggagctctgagtcgggacgatgtgtgggtcgtggcctgcctgtcggtctcctctggccgggtatgggcagaaccccacggggtgagacggggcccacggaaaccgtgtgtgcagccttccattggggaagtggggaaactgaggcccagcaagggcaggaaaccagtctaagagctgaggggtagcaggggtggggctggtgctgggcagaggccaggatggctcccaggacgtatgggcggtctgggcactgtccctcggaggcagcaacactcatggtggtgcccactgacctcacaccctgctcccccatagggaggcggcggctgccagtgccctccccaccaccaagctcccaagctcagcaggggtttcaggggcctactgcgtcattggggaaattgagactgcaagtgagaaggaggctcagtgctctgcgacttggagcatccactgagcctctgccatgagccggtgagccccactggggctggccctagggtcacggtggggtatttccagaaatcaccaggtgaggtgcaggaccagccagcgcatgggtggggcttacggtgcgaagaagaaagaggtggaggcctgccctggcccaggactcccagcgtgggggctcccggcctggccccacctctgctcctgctacatggcaggtgggcccttcctgccctggcaacctgcagggaaggccggaggggaccacccagccagggagatgttggcgtctaggaggggacaggtgtggtcccacacacccagcatcttaaagtgcgtgggtccccagcccattaggacagggtcccgggtgggcaggggtcatggtggggtgaaggtctcaggcacaggcaaggtcacaggtgcggtgagggtcttgcagggtgtgaaggtcataggtgtgcggtgaaggtcacaggtgtggggtgatggttttgggtgtggggagggtcttgcacggagcgagggtggcagcaagagctggaagctgcagggggagaatggcagcagagagcacccggccctgtgggcggcctggacagggctgggcctggggctgccggagagcctgtcagcttccaggatgggagtggcctcactcagctgctccacctccgggtcaggcaggtgagcctggggcagagaggctgagagcacctgagccacttgtgggagaggccacccccactgcccccctcaggcgaggagccggcctccagcacagcagaagggaacccccagtccccagccctagtgggagtggggaagaggcccagcaaggccccggacagaccgccagcctgtgaggtctccgctttcagttgcgttgatttgattttttctgagccttgaaggaggggtccggggcctggccctgcccaaaggcccctaggcaggccccaaagccgggacctagggtgctgagcatgacggatgttgggtttgagcggctggcttgcgacgtgagggctgaggtgtgagcctgggtatcttcagaggttcggtggacacaggcagctgcccgcggccccactgttcccgtggcctcctagtcctgctcaggcacctggtgaggaagggacgcagagggcagtgggaggtggccacgactgttccagcaggctcccctctgactcaggaattcacgggcaccacctccctggctggctctggttggtgtctggccaggttattcattatttatgctgaaagcctcttcagagtcccaggggagggtttctgtctccattcctggaggctgagagatgagggtgcagcagagtgggggcctccactccagaccctgcagtctgggctggccaagggctgcaccggtgcactgcacgtcatggctgatgaagcacttccacaccgcagcccctcagagctgccacagtcagccttagttcaccgagggggaagctgaggcccagagcatgagagggacttgcccagggccacatagtccttagcagaggaagctgtggctgggtgactcgatctttgtcctttttctttatacccgcagtctccccatagcagaggcttttcttttttttttctttttcttttttttttttttacaagaactctttatatattaaggctgttgggctgaagaagcctgagagggtggctggttctgtggagcatggtttgttgaagtacagtttgggggcctcctacactgagaataggccttttctcgtttctccaaagagtgggctggctcaagtagggcagagagagaagcctggggcagaggttagggatgggcacccagcgcctgccctcacacgctctgtgctggtgtcttcacagccacgtgccaccctgggcagcatcccctgctcaccatctggctgtgcctgtttgctgggggcacctcattcagaatccagcttattgtttccaacggccaatggccacaccctggcaggtagcaagagtaggagagaggagacacccactccgagcacaggttgggtttggagcccggccttggggcactctgtcactcaaaggcagagtggggagtgggcactgggccttaggaggtactgggtccagtgaggcagagatgcccctgccccacccccaccttgtggcttcttccctggcctggccagagctgtctggccgccatggggccctgtgtctcctgccttgacctcccagagggcagccgaggcccaggggaggcctggggacttagcctctcagggcaggacctgtctgcaggagtaggtgggtgctgggggtcccagtggtaatgaggcatcaggcagtgtgggaaggggcccatccggcccaccccagggcctctgggcaggttgcaggttgtagcgctggatctaggctcctgcccagactgtaggttcaaccaagaatggcatgggagcccagcctgctgtttgctttattaaatctgccctgtagctgggggaggggcttactttgatcatcactatgtcattgatataaaaatagaggctcagagaggtgaatgaacctgcccaaagtcacacagcaaagtgtggagatgagatactgactcagggctgtggacactgaagcctgtgctctaacgccagtggctgtcgctccctgaggcattctctcccgaacaacacagttattatattacaaaatattatcactatatttatatatcttataataccttattattacaataaaaccttattactctacctttcaaaatgaattatttaaaaagcagtatttgctcattgcagagagtctagaaactatagaaaagcaagggaaaagcaataggaccagccccaaggtcccagcatgcacagataaccttagtaatactgggacgtgtgcttcctttttaacatctgagcccgtgtaggtcctgaagcccagcttctttctaagtccattgtcatcttgaccctggagcctggccgattttgctggggaggcccttgccagccgagagcggctcctgcctgtgccggcgtggcgcgcccctctgctgaggctgggcaggacaggggctgggccagctctgtttctcacccttggctcttgtgtctctcgtttcaggaaattaaaatgcatgacagatagcgagtccgccccgcccgactggccctattacctagccattgatgggattctggccaaggtccccgagtcctgtgatggcaaactgccggacagccagccgccggggccctccacgtcccagaccgaggcgtccctgtcgccgcccgctaagtccacccctctgtacttcccgtataaccagtgctcctacgaaggccgcttcgaggatgatcgctccgacagctcctccagcttactgtcccttaagttcaggtagtgtgtctgcttgtccttcccctgccctggggtatctcagcccccaccatttagagaaagggactgggagtggcaaggccggcggcggcggccacagtggttgcagaggccgtggctgcgggcagcgcctccagggacaggcggcctcagaccagggagggctttagtgtccacaggcagaccgagtttgtctcccagctccatcacttttgagctgcacggaaagttccttgacttctctggcctcagtctccctcctataaaatgggggtaaatcagtacctttctcagagggtggctgggagcatcacaggagagaagacgcagcatggggcccggcacacggagggagaccaagccccagaccccagaatgcgccccctggcctcccttagcccacacagaccccaccctcacaggctagctgccctctcagcactggggagggtgtcgggctgcacctcatcacgtgttgccgtgggcatgacccgtcccctctgccatccatcccacacctcagacccgtcccgtgctggccacgtgactgtgcctgcaagatgctcacagggcagccgggagccaggcagcatgcaggacagacacctgcggggtgggcctggggagcccagagaaggtgcttttgaggaggggacatttggggtgggctttcaaggtaaaatagaagttggccatttggaggcaagaacaggaagattgtggatttgagtcacagcttctcccctgccctggtcttcaagtctttctgacaggaggtgtcagaaaagtatctttagtagagaaggcgtctccgaggagggtccctctcatgccgggggccgctgcttgactcaggatttctcattgaagacctgagacaaaaacgcttttgctggcagctagaaggaaccagcaggaggcctgagatttgtggctgttgttcccgtggactgagcccagttctcagactcagctgcctggggccttgcacaggactggggcgtgggggctgccctccctgatcaggcccaaagcgcggatctcacgcccctgaggttggctgtaccctctcagctcagagcagagtgtgggccagggatgagcaggcactggagcagggccctggggtctgtgggttttggcagctccctgcccttcagggaggtctgctgagaccacgggtggcccctaccccagcagcagagctctcaggaggcgcccacagggctggactgcctttactcaccacctctaccagagctctgaggtcctggggagagagcccaggcctcttgtgggccccacaccctctaggtgcctgtccttctgcctctctaccaaggtgtgccggccccatttctaggccgccgggagataagggggctcacatctcaggcccttccttctgggacctcagtttccccatctgcctaaggccgggtggggctggtggtcttggcttccctacaggggtcctgagtactctgcactacccagcaccccccacccctgccttcatctctccctgggggtggtctctccacccctggcccccaactggggctgagcccccacctgcccagtttggtgggtgaagggtgctccctggcaggatatgcccctctgcagcccagaacatcccaccctttccagaccgaaggggtgtggattgtcctgggaccctggtcattggggtcatccgctagtcgcaaaggacggcaatgcctgtggcctctctttctttctttttctttttttttttttttgagacggagtctcgctcttgtgcagagagcagtggcgcgatcttggctcactgcaacctccgcctcgtgggttcaagcgattctcctgcctcagcctcccgagtagctgggattacaggcacccgccacaacgcctggctaatttttgtatttttagtagagatggggtttcaccatgttggccaggctggtcttgaactcctgacctcaggtgatccacctgcctctgcctcccaaagtgctgggattacaggcataagcctccacacccggccacccctgttactttctgtcaaaggcggtgggttctggcccctcctttgcacatggaatatgagaccctgagtaagtgacctgactccctggggcctcagtttccccatttgcccagtaggattgtcgggagggtccggtgaggcccctggtgtgcccaggctctgtggccagcacgtccacagccggcactgtccttccaggtcggaggagcggccggtgaagaagcgcaaggtgcagagctgccacctgcagaagaagcagctgcggctgctggaggccatggtggaggagcagcgccggctgagccgcgccgtggaggagacctgccgcgaggtgcgccgcgtgctggaccagcagcacatcctgcaggtgcagagcctgcagctgcaggagcgcatgatgagtctgctggagaggatcatcaccaagtccagcgtctaggccagcaggcggcggcggcggcggggccgggcggctggtggtactgctcaggccacccagggcaggccactcaggccaggcgggcaagggggccgccccgcgagcggagaccgccttccacctggcctctggcaggatgtcccttctgaggggtattttgaggaacccccaggccctggggaccgtgaggctccagtctccagcatgaatgcccttcctcggacacaggccagggcctctggggttcactccgagtaagaacgtcctagagccactctccagtgtcgttactatcaatgatacttgacgtggctttgatattaaacgtatactttttcattcttgcctggaacgcacagtttgctgttgctggcttggtgaggatgccctgattgatggatcccgaaaatgaaagcagatggaaacgggttggggcaggctggagctgggggagctctctcctgaagggaaccctgtgtcctccctcaccaggacctctgcgtctctccttaaatggcctctgacgcctgatgaaaaccccagcgaccttccaggaggcttttattcagctctgtttggagcatcaggtgtttccactgcctccttagcaatgacactaataaaagtcgtaacacctgttcacatgcacagccctgttgagtgttctgggtgctggagatatcatggtggatgacacaaaggccctggcctcttggagcttatgctcccatgcggggaagacacatgggtcagtagagaaatggttgcaggttgtgataagtgctggaagggaggggttggcctgaggacacggaggcagacatacgtggagctgggaacagtggccacacagggaacggccagtgcgaaggcccagaggcagaggacactggagcaagcccaggagcagctaggaggctggtggccagcagccaggccacggaagcccgtgcagcccgtggggaggagtgttcatgcttttcaagcttagtgggagtcttttggccagtgcagctctgggtctgacatcggtgggggacagaggggtggtggagcggccacagctgcaagctcacctcactgccggcccttccaccagtttcaaactctttctagaagctccagctttcccaaagctgaattctctatgagcctccttggccgggactcgggcgtctggttgccctggctgcaaaggaggctggggccaggtgtgtttgagtcacctcctggaattaggcaagttgctgcccaaatagaaggttgttggcaggtgggtcagcaggtgaacagcatggtttgactcagggttcagaaaaatctccctctggctgccaagcgagcaggccgtggagacaggtgcagaggcaggtgtggcagcaggcatcctgccaggcagtgctgcagtcatcctgcgacaagcagcagcagctcatcctaccctctagggggtcttgaggtcagccaggcaagagagcagcttggactccactgggtgtgggaccagcctgtggaccatggtggtgtggagggtgccctcggcctgcctgtgtgaaggagaggccggcgtgttctgtggagcccaaaggggagctgggcaagcaggattcacttcactctgagggtcctggagctcccaccctcctcagccatctccccagagcctgtgtgccgaggactcggcccatgttgctgtgggatgagaggcagagtgtcgtgagggtgtaaggagcggcggcagtggtgggaggagggagcagcagccagcgctacggtgccagtttccagctgccagatgacgccgctgaccctgtggttgagaagagatgcacagagccagctcttgcaagccagtgtggctgccatagcacctgccgagaagcagaaggaagggtggccccaggaggacagaggatgcgggcacatctgatgcgggcctgagttttgggagcttttgctctagccagtttccagctccgggacccacccgcctcgtaggcaagacaccacccaagaaatcatttgcttaacaaacacactgggctccaactggacacctgtgccaccctagatgctgggaacccagccatgacacaggcacctgcccccagctgctgaccactgaggctggctagcagctcccatggggccagtgtggggttccccagcctcctaacagggagccagtcacaagccctcgagagggaagggtgcccgcggccctggcaggaaggttaggctggacgctcccacaagacataacagatggaggttctaaatgatgtagcaacttcttcaccctgaaactgctgtagagtcagccatgacgcaccggtacttcagtaactgccaggcatccgggacagcacaccgcgagtcgctgctgtgcttgggttagaagtggtttggtctgttttcttctcgccctctctaatcagagtcagtgattcatgcccttccatcaccttagagaaggggcaggcgctgcccgaccttctccaggctggagcagcatcgcctcatgtcagcagaactcagctgtagaatatcgtggggttggtgcctttcatcagcagcatgtccttaacaactttctgatttcttccttagttgttggtccattaaggagaaaaaaaatgatctcagccattgctaaaatatttgataagattcagcaaagcagcatgttaacattgaaaactagaatcaggagccaggcagatgtgcttgcttttcacctgtagtatttcatgttgttttgacgtttttagctaatgcattaagataaataaacaaaagccgggcacggtggttcacgcctgtaatcccagcactttgggaggctgaggcgggaggatcctctgaggtcaggagttcaagaccagcctgaccaacatggagaaacctcgtcattactaaaaatacaaaattagctgggcgtggtggtgcatgcctgtaatcccagctacttgggaggctgaggcaggagaatcgcttgaacccgggaggcggaggttgcagtgagctgagattgcaccactgcactccagcctgggtgacagtgaaactcggtctcaaaaaaaaaaaaaaattaaaaaaagataaataaaataagcaggataagaaatgaagaaagtagagttacctttgttttcagatttcatttttgtatacccagaaagccaaatgtacaaaagactgggagctctttaaaccagcttaaacttgttgaaaatgaggatgaagaaatatcccattcagagttggaatgaatttaacccagaaggaacaggacctctactgaagagaactatgcagtcttactgaaaaatctaaataatacctgagcgctggagaaacttcgcacactcctgaaagctccaaagtcaatgtcatcattttattaatgtcattccaaacatagtctcaataatatcacttcttggttttgacatggacgcgatgatgtttaaattcatatgaaaaaagaacggggccaaaagtccaaggccagtcagcgtgagaagaccgctcggcctccctcggagtcggggagttggaaccgcagactgagatcatgtggctgctggaggccaggacgaacgtcgggaaatggagactcctgcgttgctggtgggatgtggtgcagccgcttccaggagcaatttggtgtcccgtcctaaagctgaagaaacgcatttcctctggtcagtgccactcctagacaggccaccctgcggcagccgtcctcaaactggtctgaggacccctcaacgctcttaaaaatcattaaaagtgggccaggtgcggtggctcacacctgtaatcccagcactttgggaggccaagacaggcggatcacgaggtcaggacattgagatcatcctggctaacacggtgaaaccccgtctctactaaaaatacaaaaaattagccgggcgtggtggcgggcgcctgtagtcccagctacttgggaggctgagccaggagaatggcgtgaacccaggaggtggagcttgcagtgagctgagatcactccactgcactccagcctgggcagcagagcgagactctgtctcaaaaaaaaataataaataaataaataaaaataaaataaaataaaattcattaaaagtgccaaagaacttttgcttatgtgagttctaatgaccaatattaatacacattagaatatcttattagaaattaaacctgagacctttagaaaacatgtattcatttcaaaatagcaataaacccatgacatattaacataaataacaattgtatgaaaaatatattttccaaaacaaaaagttttcgggagaagtgtggcatagttttacatggtcgtaaatctctggcttaagagaagcccactggcctctcagcaggctctgggtccgtccactttgggggtgttttggttgtgaagtataggagtgaatggagaagctcattcttacccagatgtgtatttgaaaagaaaaggaacattttaataacctttgcaaataatcggtatattcttccgtgatcctattccaacactggacaggtggtggtttgttttttttttttggagacggagtcccgctctgtcactcaggctggagtgcagtggcgcgatttcagctcactgcaagctccgcctcc

SEQ ID NO: 2 - RNA, 13481 nt, Homo sapiens
  source 1..13481: mol_type = mRNA
gctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatca

SEQ ID NO: 3 - DNA, 20 bp, synthetic construct
  source 1..20: mol_type = other DNA
gggaccctgatcaacaccat

SEQ ID NO: 4 - DNA, 24 bp, synthetic construct
  source 1..24: mol_type = other DNA
ccagttcttgattttgtcgaaaca

SEQ ID NO: 5 - DNA, 18 bp, synthetic construct
  source 1..18: mol_type = other DNA
tgggtggtctccgcggcc

SEQ ID NO: 6-8 - skipped (3 entries)

SEQ ID NO: 9 - DNA, 20 bp, synthetic construct
  source 1..20: mol_type = other DNA
ccttccctgaaggttcctcc

SEQ ID NO: 10 - DNA, 15 bp, synthetic construct
  source 1..15: mol_type = other DNA
taaattgtcatcacc

SEQ ID NO: 11 - DNA, 15 bp, synthetic construct
  source 1..15: mol_type = other DNA
  modified_base 8: mod_base = OTHER; note = uracil
taaattgtcatcacc

SEQ ID NO: 12 - DNA, 15 bp, synthetic construct
  source 1..15: mol_type = other DNA
taaattgncatcacc

SEQ ID NO: 13 - DNA, 15 bp, synthetic construct
  source 1..15: mol_type = other DNA
  modified_base 5: mod_base = OTHER; note = uracil
taaattgtcatcacc

SEQ ID NO: 14 - DNA, 16 bp, synthetic construct
  source 1..16: mol_type = other DNA
taaattgtcatcacca

SEQ ID NO: 15 - DNA, 18 bp, synthetic construct
  source 1..18: mol_type = other DNA
taaattgtcatcacctta

SEQ ID NO: 16 - DNA, 20 bp, synthetic construct
  source 1..20: mol_type = other DNA
taaattgtcatcaccattta

SEQ ID NO: 17 - DNA, 17 bp, synthetic construct
  source 1..17: mol_type = other DNA
taaattgtcatcaccta

SEQ ID NO: 18 - DNA, 19 bp, synthetic construct
  source 1..19: mol_type = other DNA
taaattgtcatcaccttta

SEQ ID NO: 19 - DNA, 16 bp, synthetic construct
  source 1..16: mol_type = other DNA
gtaaattgtcatcacc

SEQ ID NO: 20 - DNA, 17 bp, synthetic construct
  source 1..17: mol_type = other DNA
ggtaaattgtcatcacc

SEQ ID NO: 21 - DNA, 18 bp, synthetic construct
  source 1..18: mol_type = other DNA
ggttaaattgtcatcacc

SEQ ID NO: 22 - DNA, 19 bp, synthetic construct
  source 1..19: mol_type = other DNA
ggtgtaaattgtcatcacc

SEQ ID NO: 23 - DNA, 20 bp, synthetic construct
  source 1..20: mol_type = other DNA
ggtgataaattgtcatcacc

SEQ ID NO: 24 - DNA, 21 bp, synthetic construct
  source 1..21: mol_type = other DNA
ggctaaattgtcatcaccgcc

SEQ ID NO: 25 - DNA, 19 bp, synthetic construct
  source 1..19: mol_type = other DNA
gctaaattgtcatcaccgc

SEQ ID NO: 26 - DNA, 21 bp, synthetic construct
  source 1..21: mol_type = other DNA
taataaattgtcatcacctta

SEQ ID NO: 27 - DNA, 19 bp, synthetic construct
  source 1..19: mol_type = other DNA
aataaattgtcatcacctt

SEQ ID NO: 28 - DNA, 21 bp, synthetic construct
  source 1..21: mol_type = other DNA
tcttaaattgtcatcaccaga

SEQ ID NO: 29 - DNA, 19 bp, synthetic construct
  source 1..19: mol_type = other DNA
cttaaattgtcatcaccag

SEQ ID NO: 30 - DNA, 18 bp, synthetic construct
  source 1..18: mol_type = other DNA
taataaattgtcatcacc

SEQ ID NO: 31 - DNA, 17 bp, synthetic construct
  source 1..17: mol_type = other DNA
aataaattgtcatcacc

SEQ ID NO: 32 - DNA, 17 bp, synthetic construct
  source 1..17: mol_type = other DNA
ataaattgtcatcacca

SEQ ID NO: 33 - DNA, 17 bp, synthetic construct
  source 1..17: mol_type = other DNA
ataaattctcatcacca

SEQ ID NO: 34 - DNA, 17 bp, synthetic construct
  source 1..17: mol_type = other DNA
ataaatagtcatcacca

SEQ ID NO: 35 - DNA, 17 bp, synthetic construct
  source 1..17: mol_type = other DNA
ataaattgtgatcacca

SEQ ID NO: 36 - DNA, 17 bp, synthetic construct
  source 1..17: mol_type = other DNA
ataaattgtcttcacca

SEQ ID NO: 37 - DNA, 17 bp, synthetic construct
  source 1..17: mol_type = other DNA
ataaattgtcaacacca

SEQ ID NO: 38 - DNA, 15 bp, synthetic construct
  source 1..15: mol_type = other DNA
taaattctcatcacc

SEQ ID NO: 39 - DNA, 15 bp, synthetic construct
  source 1..15: mol_type = other DNA
taaaatgtcatcacc

SEQ ID NO: 40 - DNA, 15 bp, synthetic construct
  source 1..15: mol_type = other DNA
taaattgtgatcacc

SEQ ID NO: 41 - DNA, 15 bp, synthetic construct
  source 1..15: mol_type = other DNA
taaattgtcttcacc

SEQ ID NO: 42 - DNA, 15 bp, synthetic construct
  source 1..15: mol_type = other DNA
taaattgtcaacacc

SEQ ID NO: 43 - DNA, 17 bp, synthetic construct
  source 1..17: mol_type = other DNA
ttaaattgtcatcacca

SEQ ID NO: 44 - DNA, 17 bp, synthetic construct
  source 1..17: mol_type = other DNA
aaaaattgtcatcacca

SEQ ID NO: 45 - DNA, 17 bp, synthetic construct
  source 1..17: mol_type = other DNA
attaattgtcatcacca

SEQ ID NO: 46 - DNA, 17 bp, synthetic construct
  source 1..17: mol_type = other DNA
atatattgtcatcacca

SEQ ID NO: 47 - DNA, 17 bp, synthetic construct
  source 1..17: mol_type = other DNA
ataatttgtcatcacca

SEQ ID NO: 48 - DNA, 17 bp, synthetic construct
  source 1..17: mol_type = other DNA
ataaattgtcatgacca

SEQ ID NO: 49 - DNA, 17 bp, synthetic construct
  source 1..17: mol_type = other DNA
ataaattgtcatctcca

SEQ ID NO: 50 - DNA, 17 bp, synthetic construct
  source 1..17: mol_type = other DNA
ataaattgtcatcagca

SEQ ID NO: 51 - DNA, 17 bp, synthetic construct
  source 1..17: mol_type = other DNA
ataaattgtcatcacga

SEQ ID NO: 52 - DNA, 17 bp, synthetic construct
  source 1..17: mol_type = other DNA
ataaattgtcatcacct

SEQ ID NO: 53 - DNA, 15 bp, synthetic construct
  source 1..15: mol_type = other DNA
taagttgtcatcacc

SEQ ID NO: 54 - DNA, 15 bp, synthetic construct
  source 1..15: mol_type = other DNA
taaagtgtcatcacc

SEQ ID NO: 55 - DNA, 15 bp, synthetic construct
  source 1..15: mol_type = other DNA
taaatggtcatcacc

SEQ ID NO: 56 - DNA, 20 bp, synthetic construct
  source 1..20: mol_type = other DNA
tctctattgcacattccaag

SEQ ID NO: 57 - DNA, 20 bp, synthetic construct
  source 1..20: mol_type = other DNA
ccttccctgaaggttcctcc

SEQ ID NO: 58 - DNA, 17 bp, synthetic construct
  source 1..17: mol_type = other DNA
aattgtcatcaccagaa

SEQ ID NO: 59 - DNA, 17 bp, synthetic construct
  source 1..17: mol_type = other DNA
aattgtcatcaccagaa

SEQ ID NO: 60 - DNA, 17 bp, synthetic construct
  source 1..17: mol_type = other DNA
aaattgtcatcaccaga

SEQ ID NO: 61 - DNA, 17 bp, synthetic construct
  source 1..17: mol_type = other DNA
taaattgtcatcaccag

SEQ ID NO: 62 - DNA, 17 bp, synthetic construct
  source 1..17: mol_type = other DNA
aataaattgtcatcacc

SEQ ID NO: 63 - DNA, 17 bp, synthetic construct
  source 1..17: mol_type = other DNA
taataaattgtcatcac

SEQ ID NO: 64 - DNA, 17 bp, synthetic construct
  source 1..17: mol_type = other DNA
ttaataaattgtcatca

SEQ ID NO: 65 - DNA, 15 bp, synthetic construct
  source 1..15: mol_type = other DNA
attgtcatcaccaga

SEQ ID NO: 66 - DNA, 15 bp, synthetic construct
  source 1..15: mol_type = other DNA
ttaataaattgtcat

SEQ ID NO: 67 - DNA, 15 bp, synthetic construct
  source 1..15: mol_type = other DNA
ttgtcatcaccagaa

SEQ ID NO: 68 - DNA, 15 bp, synthetic construct
  source 1..15: mol_type = other DNA
aattgtcatcaccag

SEQ ID NO: 69 - DNA, 15 bp, synthetic construct
  source 1..15: mol_type = other DNA
aaattgtcatcacca

SEQ ID NO: 70 - DNA, 15 bp, synthetic construct
  source 1..15: mol_type = other DNA
aataaattgtcatca

SEQ ID NO: 71 - DNA, 15 bp, synthetic construct
  source 1..15: mol_type = other DNA
attaataaattgtca

SEQ ID NO: 72 - DNA, 15 bp, synthetic construct
  source 1..15: mol_type = other DNA
tattaataaattgtc

SEQ ID NO: 73 - DNA, 15 bp, synthetic construct
  source 1..15: mol_type = other DNA
gtcatcaccagaaaa

SEQ ID NO: 74 - DNA, 15 bp, synthetic construct
  source 1..15: mol_type = other DNA
tgtcatcaccagaaa

SEQ ID NO: 75 - DNA, 15 bp, synthetic construct
  source 1..15: mol_type = other DNA
ataaattgtcatcac

SEQ ID NO: 76 - DNA, 15 bp, synthetic construct
  source 1..15: mol_type = other DNA
taataaattgtcatc

SEQ ID NO: 77 - DNA, 17 bp, synthetic construct
  source 1..17: mol_type = other DNA
ataaaatgtcatcacca

SEQ ID NO: 78 - DNA, 15 bp, synthetic construct
  source 1..15: mol_type = other DNA
taaatagtcatcacc

SEQ ID NO: 79 - DNA, 20 bp, synthetic construct
  source 1..20: mol_type = other DNA
ctcgactaaagcaggatttc

SEQ ID NO: 80 - DNA, 19 bp, synthetic construct
  source 1..19: mol_type = other DNA
aataaattgtcatcaccag

SEQ ID NO: 81 - DNA, 19 bp, synthetic construct
  source 1..19: mol_type = other DNA
cacagtgctacccaacctt

SEQ ID NO: 82 - DNA, 19 bp, synthetic construct
  source 1..19: mol_type = other DNA
tcacagctatcttctcatc

SEQ ID NO: 83 - DNA, 20 bp, synthetic construct
  source 1..20: mol_type = other DNA
tcccatttcaggagacctgg

SEQ ID NO: 84 - DNA, 20 bp, synthetic construct
  source 1..20: mol_type = other DNA
gctgattagagagaggtccc

SEQ ID NO: 85 - DNA, 16 bp, synthetic construct
  source 1..16: mol_type = other DNA
atcatggctgcagctt

SEQ ID NO: 86 - DNA, 16 bp, synthetic construct
  source 1..16: mol_type = other DNA
ttcagtcatgacttcc

SEQ ID NO: 87 - DNA, 16 bp, synthetic construct
  source 1..16: mol_type = other DNA
tggctgcagcttccga

SEQ ID NO: 88 - DNA, 16 bp, synthetic construct
  source 1..16: mol_type = other DNA
atggctgcagcttccg

SEQ ID NO: 89 - DNA, 16 bp, synthetic construct
  source 1..16: mol_type = other DNA
catggctgcagcttcc

SEQ ID NO: 90 - DNA, 16 bp, synthetic construct
  source 1..16: mol_type = other DNA
tcatggctgcagcttc

SEQ ID NO: 91 - DNA, 16 bp, synthetic construct
  source 1..16: mol_type = other DNA
catcatggctgcagct

SEQ ID NO: 92 - DNA, 16 bp, synthetic construct
  source 1..16: mol_type = other DNA
ccatcatggctgcagc

SEQ ID NO: 93 - DNA, 16 bp, synthetic construct
  source 1..16: mol_type = other DNA
tccatcatggctgcag

SEQ ID NO: 94 - DNA, 16 bp, synthetic construct
  source 1..16: mol_type = other DNA
ttccatcatggctgca

(19) United States
(12) Patent Application Publication
(10) Pub. No.: US 2024/0141343 A1
(43) Pub. Date: May 2, 2024

(54) Selective Antisense Compounds and Uses Thereof

(71) Applicant: Ionis Pharmaceuticals, Inc., Carlsbad, CA (US)

(72) Inventors: Punit P. Seth, Carlsbad, CA (US); Michael Oestergaard, Carlsbad, CA (US); Eric E. Swayze, Encinitas, CA (US)

(73) Assignee: Ionis Pharmaceuticals, Inc., Carlsbad, CA (US)

(21) Appl. No.: 18/333,007

(22) Filed: Jun. 12, 2023

Related U.S. Application Data
(63) Continuation of application No. 17/011,395, filed on Sep. 3, 2020, now Pat. No. 11,732,261.
Application No. 17/011,395 is a continuation of application No. 14/238,439, filed on Jul. 7, 2014, now abandoned.
(60) Provisional application No. 61/522,659, filed on Aug. 11, 2011.
(60) Provisional application No. 61/596,723, filed on Feb. 8, 2012.
(60) Provisional application No. 61/603,196, filed on Feb. 24, 2012.

(51) Int. Cl. C12N 15/113; C12N 15/11

(52) U.S. Cl. CPC C12N 15/113; C12N 15/111; C12N 2310/11; C12N 2310/312; C12N 2310/3125; C12N 2310/315; C12N 2310/316; C12N 2310/321; C12N 2310/3231; C12N 2310/3341; C12N 2310/341; C12N 2310/351; C12N 2320/34

(57) ABSTRACT

The present invention provides oligomeric compounds. Certain such oligomeric compounds are useful for hybridizing to a complementary nucleic acid, including but not limited, to nucleic acids in a cell. In certain embodiments, hybridization results in modulation of the amount activity or expression of the target nucleic acid in a cell.

### FIELD OF THE INVENTION

The present invention pertains generally to chemically-modified oligonucleotides for use in research, diagnostics, and/or therapeutics.

### SEQUENCE LISTING

The present application is being filed along with a Sequence Listing in electronic format. The Sequence Listing is provided as a file entitled CORE0099USC1_SEQ_ST25.txt, created Apr. 12, 2021 which is 326 Kb in size. The information in the electronic format of the sequence listing is incorporated herein by reference in its entirety.

### BACKGROUND OF THE INVENTION

Antisense compounds have been used to modulate target nucleic acids. Antisense compounds comprising a variety of chemical modifications and motifs have been reported. In certain instances, such compounds are useful as research tools, diagnostic reagents, and as therapeutic agents. In certain instances antisense compounds have been shown to modulate protein expression by binding to a target messenger RNA (mRNA) encoding the protein. In certain instances, such binding of an antisense compound to its target mRNA results in cleavage of the mRNA. Antisense compounds that modulate processing of a pre-mRNA have also been reported. Such antisense compounds alter splicing, interfere with polyadenylation or prevent formation of the 5′-cap of a pre-mRNA.

### SUMMARY OF THE INVENTION

In certain embodiments, the present invention provides oligomeric compounds comprising oligonucleotides. In certain embodiments, such oligonucleotides comprise a region having a gapmer motif. In certain embodiments, such oligonucleotides consist of a region having a gapmer motif.

The present disclosure provides the following non-limiting numbered embodiments:
Embodiment 1: A oligomeric compound comprising a modified oligonucleotide consisting of 10 to 30 linked nucleosides, wherein the modified oligonucleotide has a modification motif comprising: a 5′-region consisting of 2-8 linked 5′-region nucleosides, each independently selected from among a modified nucleoside and an unmodified deoxynucleoside, provided that at least one 5′-region nucleoside is a modified nucleoside and wherein the 3′-most 5′-region nucleoside is a modified nucleoside;
   a 3′-region consisting of 2-8 linked 3′-region nucleosides, each independently selected from among a modified nucleoside and an unmodified deoxynucleoside, provided that at least one 3′-region nucleoside is a modified nucleoside and wherein the 5′-most 3′-region nucleoside is a modified nucleoside; and
   a central region between the 5′-region and the 3′-region consisting of 6-12 linked central region nucleosides, each independently selected from among: a modified nucleoside and an unmodified deoxynucleoside, wherein the 5′-most central region nucleoside is an unmodified deoxynucleoside and the 3′-most central region nucleoside is an unmodified deoxynucleoside;
   wherein the modified oligonucleotide has a nucleobase sequence complementary to the nucleobase sequence of a target region of a target nucleic acid.
Embodiment 2: The oligomeric compound of embodiment 1, wherein the nucleobase sequence of the target region of the target nucleic acid differs from the nucleobase sequence of at least one non-target nucleic acid by 1-3 differentiating nucleobases.
Embodiment 3: The oligomeric compound of embodiment 1, the nucleobase sequence of the target region of the target nucleic acid differs from the nucleobase sequence of at least one non-target nucleic acid by a single differentiating nucleobase.
Embodiment 4: The oligomeric compound of embodiment 2 or 3, wherein the target nucleic acid and the non-target nucleic acid are alleles of the same gene.
Embodiment 5: The oligomeric compound of embodiment 4, wherein the single differentiating nucleobase is a single-nucleotide polymorphism.
Embodiment 6: The oligomeric compound of embodiment 5, wherein the single-nucleotide polymorphism is associated with a disease.
Embodiment 7: The oligomeric compound of embodiment 6, wherein the disease is selected from among: Alzheimer's disease, Creutzfeldt-Jakob disease, fatal familial insomnia, Alexander disease, Parkinson's disease, amyotrophic lateral sclerosis, dentato-rubral and pallido-luysian atrophy DRPA, spino-cerebellar ataxia, Torsion dystonia, cardiomyopathy, chronic obstructive pulmonary disease (COPD), liver disease, hepatocellular carcinoma, systemic lupus erythematosus, hypercholesterolemia, breast cancer, asthma, Type 1 diabetes, Rheumatoid arthritis, Graves disease, SLE, spinal and bulbar muscular atrophy, Kennedy's disease, progressive childhood posterior subcapsular cataracts, cholesterol gallstone disease, arthrosclerosis, cardiovascular disease, primary hypercalciuria, alpha-thallasemia, obsessive compulsive disorder, Anxiety, comorbid depression, congenital visual defects, hypertension, metabolic syndrome, prostate cancer, congential myasthenic syndrome, peripheral arterial disease, atrial fibrillation, sporadic pheochromocytoma, congenital malformations, Machado-Joseph disease, Huntington's disease, and Autosomal Dominant Retinitis Pigmentosa disease.
Embodiment 8: The oligomeric compound of embodiment 6, wherein the single-nucleotide polymorphism is selected from among: rs6446723, rs3856973, rs2285086, rs363092, rs916171, rs6844859, rs7691627, rs4690073, rs2024115, rs11731237, rs362296, rs10015979, rs7659144, rs363096, rs362273, rs16843804, rs362271, rs362275, rs3121419, rs362272, rs3775061, rs34315806, rs363099, rs2298967, rs363088, rs363064, rs363102, rs2798235, rs363080, rs363072, rs363125, rs362303, rs362310, rs10488840, rs362325, rs35892913, rs363102, rs363096, rs11731237, rs10015979, rs363080, rs2798235, rs1936032, rs2276881, rs363070, rs35892913, rs12502045, rs6446723, rs7685686, rs3733217, rs6844859, and rs362331.
Embodiment 9: The oligomeric compound of embodiment 8, wherein the single-nucleotide polymorphism is selected from among: rs7685686, rs362303 rs4690072 and rs363088 Embodiment 10: The oligomeric compound of embodiment 2 or 3, wherein the target nucleic acid and the non-target nucleic acid are transcripts from different genes.
Embodiment 11: The oligomeric compound of any of embodiments 1-10, wherein the 3′-most 5′-region nucleoside comprises a bicyclic sugar moiety.
Embodiment 12: The oligomeric compound of embodiment 11, wherein the 3′-most 5′-region nucleoside comprises a cEt sugar moiety.
Embodiment 13: The oligomeric compound of embodiment 11, wherein the 3′-most 5′-region nucleoside comprises an LNA sugar moiety.
Embodiment 14: The oligomeric compound of any of embodiments 1-13, wherein the central region consists of 6-10 linked nucleosides.
Embodiment 15: The oligomeric compound of any of embodiments 1-14, wherein the central region consists of 6-9 linked nucleosides.
Embodiment 16: The oligomeric compound of embodiment 15, wherein the central region consists of 6 linked nucleosides.
Embodiment 17: The oligomeric compound of embodiment 15, wherein the central region consists of 7 linked nucleosides.
Embodiment 18: The oligomeric compound of embodiment 15, wherein the central region consists of 8 linked nucleosides.
Embodiment 19: The oligomeric compound of embodiment 15, wherein the central region consists of 9 linked nucleosides.
Embodiment 20: The oligomeric compound of any of embodiments 1-19, wherein each central region nucleoside is an unmodified deoxynucleoside.
Embodiment 21: The oligomeric compound of any of embodiments 1-19, wherein at least one central region nucleoside is a modified nucleoside.
Embodiment 22: The oligomeric compound of embodiment 21, wherein one central region nucleoside is a modified nucleoside and each of the other central region nucleosides is an unmodified deoxynucleoside.
Embodiment 23: The oligomeric compound of embodiment 21, wherein two central region nucleosides are modified nucleosides and each of the other central region nucleosides is an unmodified deoxynucleoside.
Embodiment 24: The oligomeric compound of any of embodiments 21-23 wherein at least one modified central region nucleoside is an RNA-like nucleoside.
Embodiment 25: The oligomeric compound of any of embodiments 21-23 comprising at least one modified central region nucleoside comprising a modified sugar moiety.
Embodiment 26: The oligomeric compound of any of embodiments 21-25 comprising at least one modified central region nucleoside comprising a 5′-methyl-2′-deoxy sugar moiety.
Embodiment 27: The oligomeric compound of any of embodiments 21-26 comprising at least one modified central region nucleoside comprising a bicyclic sugar moiety.
Embodiment 28: The oligomeric compound of any of embodiments 21-27 comprising at least one modified central region nucleoside comprising a cEt sugar moiety.
Embodiment 29: The oligomeric compound of any of embodiments 21-28 comprising at least one modified central region nucleoside comprising an LNA sugar moiety.
Embodiment 30: The oligomeric compound of any of embodiments 21-29 comprising at least one modified central region nucleoside comprising an α-LNA sugar moiety.
Embodiment 31: The oligomeric compound of any of embodiments 21-29 comprising at least one modified central region nucleoside comprising a 2′-substituted sugar moiety.
Embodiment 32: The oligomeric compound of embodiment 31 wherein at least one modified central region nucleoside comprises a 2′-substituted sugar moiety comprising a 2′ substituent selected from among: halogen, optionally substituted allyl, optionally substituted amino, azido, optionally substituted SH, CN, OCN, CF3, OCF3, O, S, or N(Rm)-alkyl; O, S, or N(Rm)-alkenyl; O, S or N(Rm)-alkynyl; optionally substituted O-alkylenyl-O-alkyl, optionally substituted alkynyl, optionally substituted alkaryl, optionally substituted aralkyl, optionally substituted O-alkaryl, optionally substituted O-aralkyl, O(CH2)2SCH3, O—(CH2)2-O—N(Rm)(Rn) or O—CH2-C(═O)—N(Rm)(Rn), where each Rm and Rn is, independently, H, an amino protecting group or substituted or unsubstituted C₁-C₁₀ alkyl;
   wherein each optionally substituted group is optionally substituted with a substituent group independently selected from among: hydroxyl, amino, alkoxy, carboxy, benzyl, phenyl, nitro (NO₂), thiol, thioalkoxy (S-alkyl), halogen, alkyl, aryl, alkenyl and alkynyl.
Embodiment 33: The oligomeric compound of embodiment 32 wherein at least one modified central region nucleoside comprises a 2′-substituted sugar moiety comprising a 2′ substituent selected from among: a halogen, OCH₃, OCH₂F, OCHF₂, OCF₃, OCH₂CH₃, O(CH₂)₂F, OCH₂CHF₂, OCH₂CF₃, OCH₂—CH═CH₂, O(CH₂)₂—OCH₃, O(CH₂)₂—SCH₃, O(CH₂)₂—OCF₃, O(CH₂)₃—N(R₁)(R₂), O(CH₂)₂—ON(R₁)(R₂), O(CH₂)₂—O(CH₂)₂—N(R₁)(R₂), OCH₂C(═O)—N(R₁)(R₂), OCH₂C(═O)—N(R₃)—(CH₂)₂—N(R₁)(R₂), and O(CH₂)₂—N(R₃)—C(═NR₄)[N(R₁)(R₂)]; wherein R₁, R₂, R₃ and R₄ are each, independently, H or C₁-C₆ alkyl.
Embodiment 34: The oligomeric compound of embodiment 33 wherein the 2′ substituent is selected from among: a halogen, OCH₃, OCF₃, OCH₂CH₃, OCH₂CF₃, OCH₂—CH═CH₂, O(CH₂)₂—OCH₃ (MOE), O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂.
Embodiment 35: The oligomeric compound of any of embodiments 21-34 comprising at least one modified central region nucleoside comprising a 2′-MOE sugar moiety.
Embodiment 36: The oligomeric compound of any of embodiments 21-35 comprising at least one modified central region nucleoside comprising a 2′-OMe sugar moiety.
Embodiment 37: The oligomeric compound of any of embodiments 21-36 comprising at least one modified central region nucleoside comprising a 2′-F sugar moiety.
Embodiment 38: The oligomeric compound of any of embodiments 21-37 comprising at least one modified central region nucleoside comprising a 2′-(ara)-F sugar moiety.
Embodiment 39: The oligomeric compound of any of embodiments 21-38 comprising at least one modified central region nucleoside comprising a sugar surrogate.
Embodiment 40: The oligomeric compound of embodiment 39 comprising at least one modified central region nucleoside comprising an F-HNA sugar moiety.
Embodiment 41: The oligomeric compound of embodiment 39 or 40 comprising at least one modified central region nucleoside comprising an HNA sugar moiety.
Embodiment 42: The oligomeric compound of any of embodiments 21-41 comprising at least one modified central region nucleoside comprising a modified nucleobase.
Embodiment 43: The oligomeric compound of embodiment 42 comprising at least one modified central region nucleoside comprising a modified nucleobase selected from a 2-thio pyrimidine and a 5-propyne pyrimidine.
Embodiment 44: The oligomeric compound of any of embodiments 21-43, wherein the 2^{nd} nucleoside from the 5′-end of the central region is a modified nucleoside.
Embodiment 45: The oligomeric compound of any of embodiments 21-44, wherein the 3^{rd} nucleoside from the 5′-end of the central region is a modified nucleoside.
Embodiment 46: The oligomeric compound of any of embodiments 21-45, wherein the 4^{th} nucleoside from the 5′-end of the central region is a modified nucleoside.
Embodiment 47: The oligomeric compound of any of embodiments 21-46, wherein the 5^{th} nucleoside from the 5′-end of the central region is a modified nucleoside.
Embodiment 48: The oligomeric compound of any of embodiments 21-47, wherein the 6^{th} nucleoside from the 5′-end of the central region is a modified nucleoside.
Embodiment 49: The oligomeric compound of any of embodiments 21-48, wherein the 8^{th} nucleoside from the 3′-end of the central region is a modified nucleoside.
Embodiment 50: The oligomeric compound of any of embodiments 21-49, wherein the 7^{th} nucleoside from the 3′-end of the central region is a modified nucleoside.
Embodiment 51: The oligomeric compound of any of embodiments 21-50, wherein the 6^{th} nucleoside from the 3′-end of the central region is a modified nucleoside.
Embodiment 52: The oligomeric compound of any of embodiments 21-51, wherein the 5^{th} nucleoside from the 3′-end of the central region is a modified nucleoside.
Embodiment 53: The oligomeric compound of any of embodiments 21-52, wherein the 4^{th} nucleoside from the 3′-end of the central region is a modified nucleoside.
Embodiment 54: The oligomeric compound of any of embodiments 21-53, wherein the 3^{rd} nucleoside from the 3′-end of the central region is a modified nucleoside.
Embodiment 55: The oligomeric compound of any of embodiments 21-54, wherein the 2^{nd} nucleoside from the 3′-end of the central region is a modified nucleoside.
Embodiment 56: The oligomeric compound of any of embodiments 21-55, wherein the modified nucleoside is a 5′-methyl-2′-deoxy sugar moiety.
Embodiment 57: The oligomeric compound of any of embodiments 21-55, wherein the modified nucleoside is a 2-thio pyrimidine.
Embodiment 58: The oligomeric compound of any of embodiments 21-55, wherein the central region comprises no region having more than 4 contiguous unmodified deoxynucleosides.
Embodiment 59: The oligomeric compound of any of embodiments 21-55, wherein the central region comprises no region having more than 5 contiguous unmodified deoxynucleosides.
Embodiment 60: The oligomeric compound of any of embodiments 21-55, wherein the central region comprises no region having more than 6 contiguous unmodified deoxynucleosides.
Embodiment 61: The oligomeric compound of any of embodiments 21-55, wherein the central region comprises no region having more than 7 contiguous unmodified deoxynucleosides.
Embodiment 62: The oligomeric compound of any of embodiments 1-14 or 21-59, wherein the central region has a nucleoside motif selected from among: DDDDDDDDDD, DDDDXDDDDD; DDDDDXDDDDD; DDDXDDDDD; DDDDXDDDDDD; DDDDXDDDD; DDXDDDDDD; DDDXDDDDDD; DXDDDDDD; DDXDDDDDDD; DDXDDDDD; DDXDDDXDDD; DDDXDDDXDDD; DXDDDXDDD; DDXDDDXDD; DDXDDDDXDDD; DDXDDDDXDD; DXDDDDXDDD; DDDDXDDD; DDDXDDD; DXDDDDDDD; DDDDXXDDD; and DXXDXXDXX; wherein
   each D is an unmodified deoxynucleoside; and each X is a modified nucleoside.
Embodiment 63: The oligomeric compound of any of embodiments 1-14 or 21-59, wherein the central region has a nucleoside motif selected from among: DDDDDDDDD; DXDDDDDDD; DDXDDDDDD; DDDXDDDDD; DDDDXDDDD; DDDDDXDDD; DDDDDDXDD; DDDDDDDXD; DXXDDDDDD; DDDDDDXXD; DDXXDDDDD; DDDXXDDDD; DDDDXXDDD; DDDDDXXDD; DXDDDDDXD; DXDDDDXDD; DXDDDXDDD; DXDDXDDDD; DXDXDDDDD; DDXDDDDXD; DDXDDDXDD; DDXDDXDDD; DDXDXDDDD; DDDXDDDXD; DDDXDDXDD; DDDXDXDDD; DDDDXDDXD; DDDDXDXDD; and DDDDDXDXD wherein each D is an unmodified deoxynucleoside; and each X is a modified nucleoside.
Embodiment 64: The oligomeric compound of any of embodiments 1-14 or 21-59, wherein the central region has a nucleoside motif selected from among: DDDDXDDDD, DXDDDDDDD, DXXDDDDDD, DDXDDDDDD, DDDXDDDDD, DDDDXDDDD, DDDDDXDDD, DDDDDDXDD, and DDDDDDDXD.
Embodiment 65: The oligomeric compound of any of embodiments 1-14 or 21-59, wherein the central region has a nucleoside motif selected from among: DDDDDDDD, DXDDDDDD, DDXDDDDD, DDDXDDDD, DDDDXDDD, DDDDDXDD, DDDDDDXD, DXDDDDXD, DXDDDXDD, DXDDXDDD, DXDXDDDD, DXXDDDDD, DDXXDDDD, DDXDXDDD, DDXDDXDD, DXDDDDXD, DDDXXDDD, DDDXDXDD, DDDXDDXD, DDDDXXDD, DDDDXDXD, and DDDDDXXD.
Embodiment 66: The oligomeric compound of any of embodiments 1-14 or 21-59, wherein the central region has a nucleoside motif selected from among: DDDDDDD, DXDDDDD, DDXDDDD, DDDXDDD, DDDDXDD, DDDDDXD, DXDDDXD, DXDDXDD, DXDXDDD, DXXDDDD, DDXXDDD, DDXDXDD, DDXDDXD, DDDXXDD, DDDXDXD, and DDDDXXD.
Embodiment 67: The oligomeric compound of any of embodiments 1-14 or 21-59, wherein the central region has a nucleoside motif selected from among: DDDDDD, DXDDDD, DDXDDD, DDDXDD, DDDDXD, DXXDDD, DXDXDD, DXDDXD, DDXXDD, DDXDXD, and DDDXXD.
Embodiment 68: The oligomeric compound of any of embodiments 1-14 or 21-59, wherein the central region has a nucleoside motif selected from among: DDDDDD, DDDDDDD, DDDDDDDD, DDDDDDDDD, DXDDDD, DDXDDD, DDDXDD, DDDDXD, DXDDDDD, DDXDDDD, DDDXDDD, DDDDXDD, DDDDDXD, DXDDDDDD, DDXDDDDD, DDDXDDDD, DDDDXDDD, DDDDDXDD, DDDDDDXD, DXDDDDDDD; DDXDDDDDD, DDDXDDDDD, DDDDXDDDD, DDDDDXDDD, DDDDDDXDD, DDDDDDDXD, DXDDDDDDDD, DDXDDDDDDD, DDDXDDDDDD, DDDDXDDDDD, DDDDDXDDDD, DDDDDDXDDD, DDDDDDDXDD, and DDDDDDDDXD.
Embodiment 69: The oligomeric compound of embodiments 62-68, wherein each X comprises a modified nucleobase.
Embodiment 70: The oligomeric compound of embodiments 62-68, wherein each X comprises a modified sugar moiety.
Embodiment 71: The oligomeric compound of embodiments 62-68, wherein each X comprises 2-thio-thymidine.
Embodiment 72: The oligomeric compound of embodiments 62-68, wherein each X nucleoside comprises an F-HNA sugar moiety.
Embodiment 73: The oligomeric compound of embodiments 62-68, wherein the nucleobase sequence of the target region of the target nucleic acid differs from the nucleobase sequence of at least one non-target nucleic acid by a single differentiating nucleobase, and wherein the location of the single differentiating nucleobase is represented by X.
Embodiment 74: The oligomeric compound of embodiment 73, wherein the target nucleic acid and the non-target nucleic acid are alleles of the same gene.
Embodiment 75: The oligomeric compound of embodiment 73, wherein the single differentiating nucleobase is a single-nucleotide polymorphism.
Embodiment 76: The oligomeric compound of any of embodiments 1-75, wherein the 5′ region consists of 2 linked 5′-region nucleosides.
Embodiment 77: The oligomeric compound of any of embodiments 1-75, wherein the 5′ region consists of 3 linked 5′-region nucleosides.
Embodiment 78: The oligomeric compound of any of embodiments 1-75, wherein the 5′ region consists of 4 linked 5′-region nucleosides.
Embodiment 79: The oligomeric compound of any of embodiments 1-75, wherein the 5′ region consists of 5 linked 5′-region nucleosides.
Embodiment 80: The oligomeric compound of any of embodiments 1-75, wherein the 5′ region consists of 6 linked 5′-region nucleosides.
Embodiment 81: The oligomeric compound of any of embodiments 1-80, wherein at least one 5′-region nucleoside is an unmodified deoxynucleoside.
Embodiment 82: The oligomeric compound of any of embodiments 1-80, wherein each 5′-region nucleoside is a modified nucleoside.
Embodiment 83: The oligomeric compound of any of embodiments 1-80 wherein at least one 5′-region nucleoside is an RNA-like nucleoside.
Embodiment 84: The oligomeric compound of any of embodiments 1-80 wherein each 5′-region nucleoside is an RNA-like nucleoside.
Embodiment 85: The oligomeric compound of any of embodiments 1-80 comprising at least one modified 5′-region nucleoside comprising a modified sugar.
Embodiment 86: The oligomeric compound of embodiment 80 comprising at least one modified 5′-region nucleoside comprising a bicyclic sugar moiety.
Embodiment 87: The oligomeric compound of embodiment 86 comprising at least one modified 5′-region nucleoside comprising a cEt sugar moiety.
Embodiment 88: The oligomeric compound of embodiment 85 or 86 comprising at least one modified 5′-region nucleoside comprising an LNA sugar moiety.
Embodiment 89: The oligomeric compound of any of embodiments 76-80 comprising of at least one modified 5′-region nucleoside comprising a 2′-substituted sugar moiety.
Embodiment 90: The oligomeric compound of embodiment 89 wherein at least one modified central region nucleoside comprises a 2′-substituted sugar moiety comprising a 2′ substituent selected from among: halogen, optionally substituted allyl, optionally substituted amino, azido, optionally substituted SH, CN, OCN, CF₃, OCF₃, O, S, or N(Rₘ)-alkyl; O, S, or N(Rₘ)-alkenyl; O, S or N(Rₘ)-alkynyl; optionally substituted O-alkylenyl-O-alkyl, optionally substituted alkynyl, optionally substituted alkaryl, optionally substituted aralkyl, optionally substituted O-alkaryl, optionally substituted O-aralkyl, O(CH₂)₂SCH₃, O—(CH₂)₂—O—N(Rₘ)(Rn) or O—CH₂—C(═O)—N(Rₘ)(Rₙ), where each Rₘ and Rₙ is, independently, H, an amino protecting group or substituted or unsubstituted C₁-C₁₀ alkyl;
wherein each optionally substituted group is optionally substituted with a substituent group independently selected from among: hydroxyl, amino, alkoxy, carboxy, benzyl, phenyl, nitro (NO₂), thiol, thioalkoxy (S-alkyl), halogen, alkyl, aryl, alkenyl and alkynyl.
Embodiment 91: The oligomeric compound of embodiment 90 wherein at least one modified 5′-region nucleoside comprises a 2′-substituted sugar moiety comprising a 2′-substituent selected from among: a halogen, OCH₃, OCH₂F, OCHF₂, OCF₃, OCH₂CH₃, O(CH₂)₂F, OCH₂CHF₂, OCH₂CF₃, OCH₂—CH═CH₂, O(CH₂)₂—OCH₃ (MOE), O(CH₂)₂—SCH₃, O(CH₂)₂—OCF₃, O(CH₂)₃—N(R₁)(R₂), O(CH₂)₂—ON(R₁)(R₂), O(CH₂)₂—O(CH₂)₂—N(R₁)(R₂), OCH₂C(═O)—N(R₁)(R₂), OCH₂C(═O)—N(R₃)—(CH₂)₂—N(R₁)(R₂), and O(CH₂)₂—N(R₃)—C(═NR₄)[N(R₁)(R₂)]; wherein R₁, R₂, R₃ and R₄ are each, independently, H or C₁-C₆ alkyl.
Embodiment 92: The oligomeric compound of embodiment 91, wherein the 2′-substituent is selected from among: a halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂.
Embodiment 93: The oligomeric compound of any of embodiments 89-92 comprising at least one modified 5′-region nucleoside comprising a 2′-MOE sugar moiety.
Embodiment 94: The oligomeric compound of any of embodiments 89-92 comprising at least one modified 5′-region nucleoside comprising a 2′-OMe sugar moiety.
Embodiment 95: The oligomeric compound of any of embodiments 89-92 comprising at least one modified 5′-region nucleoside comprising a 2′-F sugar moiety.
Embodiment 96: The oligomeric compound of any of embodiments 89-92 comprising at least one modified 5′-region nucleoside comprising a 2′-(ara)-F sugar moiety.
Embodiment 97: The oligomeric compound of any of embodiments 82-96 comprising of at least one modified 5′-region nucleoside comprising a sugar surrogate.
Embodiment 98: The oligomeric compound of embodiment 97 comprising at least one modified 5′-region nucleoside comprising an F-HNA sugar moiety.
Embodiment 99: The oligomeric compound of embodiment 97 or 98 comprising at least one modified 5′-region nucleoside comprising an HNA sugar moiety.
Embodiment 100: The oligomeric compound of any of embodiments 1-99 comprising at least one modified 5′-region nucleoside comprising a modified nucleobase.
Embodiment 101: The oligomeric compound of embodiment 100, wherein the modified nucleoside comprises 2-thio-thymidine.
Embodiment 102: The oligomeric compound of any of embodiments 1-101, wherein the 5′-region has a motif selected from among:
   ADDA; ABDAA; ABBA; ABB; ABAA; AABAA; AAABAA; AAAABAA; AAAAABAA; AAABAA; AABAA; ABAB; ABADB; ABADDB; AAABB; AAAAA; ABBDC; ABDDC; ABBDCC; ABBDDC; ABBDCC; ABBC; AA; AAA; AAAA; AAAAB; AAAAAAA; AAAAAAAA; ABBB; AB; ABAB; AAAAB; AABBB; AAAAB; and AABBB,
   wherein each A is a modified nucleoside of a first type, each B is a modified nucleoside of a second type, each C is a modified nucleoside of a third type, and each D is an unmodified deoxynucleoside.
Embodiment 103: The oligomeric compound of any of embodiments 1-101, wherein the 5′-region has a motif selected from among:
   AB, ABB, AAA, BBB, BBBAA, AAB, BAA, BBAA, AABB, AAAB, ABBW, ABBWW, ABBB, ABBBB, ABAB, ABABAB, ABABBB, ABABAA, AAABB, AAAABB, AABB, AAAAB, AABBB, ABBBB, BBBBB, AAABW, AAAAA, BBBBAA, and AAABW wherein each A is a modified nucleoside of a first type, each B is a modified nucleoside of a second type, and each W is a modified nucleoside of a third type.
Embodiment 104: The oligomeric compound of any of embodiments 1-101, wherein the 5′-region has a motif selected from among: ABB; ABAA; AABAA; AAABAA; ABAB; ABADB; AAABB; AAAAA; AA; AAA; AAAA; AAAAB; ABBB; AB; and ABAB, wherein each A is a modified nucleoside of a first type, each B is a modified nucleoside of a second type, and each W is a modified nucleoside of a third type.
Embodiment 105: The oligomeric compound of embodiments 102-104, wherein each A nucleoside comprises a 2′-substituted sugar moiety.
Embodiment 106: The oligomeric compound of embodiment 105 wherein at least one central region nucleoside comprises a 2′-substituted sugar moiety comprising a 2′ substituent selected from among: halogen, optionally substituted allyl, optionally substituted amino, azido, optionally substituted SH, CN, OCN, CF₃, OCF₃, O, S, or N(Rₘ)-alkyl; O, S, or N(Rₘ)-alkenyl; O, S or N(Rₘ)-alkynyl; optionally substituted O-alkylenyl-O-alkyl, optionally substituted alkynyl, optionally substituted alkaryl, optionally substituted aralkyl, optionally substituted O-alkaryl, optionally substituted O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl; wherein each optionally substituted group is optionally substituted with a substituent group independently selected from among: hydroxyl, amino, alkoxy, carboxy, benzyl, phenyl, nitro (NO₂), thiol, thioalkoxy (S-alkyl), halogen, alkyl, aryl, alkenyl and alkynyl.
Embodiment 107: The oligomeric compound of embodiment 102-106, wherein each A nucleoside comprises a 2′-substituted sugar moiety comprising a 2′-substituent selected from among: a halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂.
Embodiment 108: The oligomeric compound of embodiment 107, wherein each A nucleoside comprises a 2′-substituted sugar moiety comprising a 2′-substituent selected from among: F, OCH₃, O(CH₂)₂—OCH₃.
Embodiment 109: The oligomeric compound of embodiments 102-106, wherein each A nucleoside comprises a bicyclic sugar moiety.
Embodiment 110: The oligomeric compound of embodiment 109, wherein each A nucleoside comprises a bicyclic sugar moiety selected from among: cEt, cMOE, LNA, α-LNA, ENA and 2′-thio LNA.
Embodiment 111: The oligomeric compound of any of embodiments 102-110, wherein each A comprises a modified nucleobase.
Embodiment 112: The oligomeric compound of embodiment 111, wherein each A comprises a modified nucleobase selected from among a 2-thio pyrimidine and a 5-propyne pyrimidine.
Embodiment 113: The oligomeric compound of embodiment 112, wherein each A comprises 2-thio-thymidine.
Embodiment 114: The oligomeric compound of embodiment 102-106, wherein each A nucleoside comprises an unmodified 2′-deoxyfuranose sugar moiety.
Embodiment 115: The oligomeric compound of embodiment 102-106, wherein each A nucleoside comprises an F-HNA sugar moiety.
Embodiment 116: The oligomeric compound of any of embodiments 102-115, wherein each B nucleoside comprises a 2′-substituted sugar moiety.
Embodiment 117: The oligomeric compound of embodiment 116, wherein at least one central region nucleoside comprises a 2′-substituted sugar moiety comprising a 2′ substituent selected from among: halogen, optionally substituted allyl, optionally substituted amino, azido, optionally substituted SH, CN, OCN, CF₃, OCF₃, O, S, or N(Rₘ)-alkyl; O, S, or N(Rₘ)-alkenyl; O, S or N(Rₘ)-alkynyl; optionally substituted O-alkylenyl-O-alkyl, optionally substituted alkynyl, optionally substituted alkaryl, optionally substituted aralkyl, optionally substituted O-alkaryl, optionally substituted O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl; wherein each optionally substituted group is optionally substituted with a substituent group independently selected from among: hydroxyl, amino, alkoxy, carboxy, benzyl, phenyl, nitro (NO₂), thiol, thioalkoxy (S-alkyl), halogen, alkyl, aryl, alkenyl and alkynyl.
Embodiment 118: The oligomeric compound of embodiment 117, wherein each B nucleoside comprises a 2′-substituted sugar moiety comprising a 2′-substituent selected from among: a halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂.
Embodiment 119: The oligomeric compound of embodiment 118, wherein each B nucleoside comprises a 2′-substituted sugar moiety comprising a 2′-substituent selected from among: F, OCH₃, O(CH₂)₂—OCH₃.
Embodiment 120: The oligomeric compound of any of embodiments 102-115, wherein each B nucleoside comprises a bicyclic sugar moiety.
Embodiment 121: The oligomeric compound of embodiment 120, wherein each B nucleoside comprises a bicyclic sugar moiety selected from among: cEt, cMOE, LNA, α-LNA, ENA and 2′-thio LNA.
Embodiment 122: The oligomeric compound of any of embodiments 102-115, wherein each B comprises a modified nucleobase.
Embodiment 123: The oligomeric compound of embodiment 122, wherein each B comprises a modified nucleobase selected from among a 2-thio pyrimidine and a 5-propyne pyrimidine.
Embodiment 124: The oligomeric compound of embodiment 123, wherein each B comprises 2-thio-thymidine.
Embodiment 125: The oligomeric compound of embodiment 102-106, wherein each B nucleoside comprises an unmodified 2′-deoxyfuranose sugar moiety.
Embodiment 126: The oligomeric compound of embodiment 102-115, wherein each B nucleoside comprises an F-HNA sugar moiety.
Embodiment 127: The oligomeric compound of any of embodiments 102-126, wherein each C nucleoside comprises a 2′-substituted sugar moiety.
Embodiment 128: The oligomeric compound of embodiment 127, wherein at least one central region nucleoside comprises a 2′-substituted sugar moiety comprising a 2′ substituent selected from among: halogen, optionally substituted allyl, optionally substituted amino, azido, optionally substituted SH, CN, OCN, CF₃, OCF₃, O, S, or N(Rₘ)-alkyl; O, S, or N(Rₘ)-alkenyl; O, S or N(Rₘ)-alkynyl; optionally substituted O-alkylenyl-O-alkyl, optionally substituted alkynyl, optionally substituted alkaryl, optionally substituted aralkyl, optionally substituted O-alkaryl, optionally substituted O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl; wherein each optionally substituted group is optionally substituted with a substituent group independently selected from among: hydroxyl, amino, alkoxy, carboxy, benzyl, phenyl, nitro (NO₂), thiol, thioalkoxy (S-alkyl), halogen, alkyl, aryl, alkenyl and alkynyl.
Embodiment 129: The oligomeric compound of embodiment 128, wherein each C nucleoside comprises a 2′-substituted sugar moiety comprising a 2′-substituent selected from among: a halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂.
Embodiment 130: The oligomeric compound of embodiment 129, wherein each C nucleoside comprises a 2′-substituted sugar moiety comprising a 2′-substituent selected from among: F, OCH₃, O(CH₂)₂—OCH₃.
Embodiment 131: The oligomeric compound of any of embodiments 102-126, wherein each C nucleoside comprises a bicyclic sugar moiety.
Embodiment 132: The oligomeric compound of embodiment 131, wherein each C nucleoside comprises a bicyclic sugar moiety selected from among: cEt, cMOE, LNA, α-LNA, ENA and 2′-thio LNA.
Embodiment 133: The oligomeric compound of any of embodiments 102-126, wherein each C comprises a modified nucleobase.
Embodiment 134: The oligomeric compound of embodiment 133, wherein each C comprises a modified nucleobase selected from among a 2-thio pyrimidine and a 5-propyne pyrimidine.
Embodiment 135: The oligomeric compound of embodiment 134, wherein each C comprises 2-thio-thymidine.
Embodiment 136: The oligomeric compound of embodiment 102-126, wherein each C comprises an F-HNA sugar moiety.
Embodiment 137: The oligomeric compound of embodiment 102-126, wherein each C nucleoside comprises an unmodified 2′-deoxyfuranose sugar moiety.
Embodiment 138: The oligomeric compound of any of embodiments 102-138, wherein each W nucleoside comprises a 2′-substituted sugar moiety.
Embodiment 139: The oligomeric compound of embodiment 138, wherein at least one central region nucleoside comprises a 2′-substituted sugar moiety comprising a 2′ substituent selected from among: halogen, optionally substituted allyl, optionally substituted amino, azido, optionally substituted SH, CN, OCN, CF₃, OCF₃, O, S, or N(Rₘ)-alkyl; O, S, or N(Rₘ)-alkenyl; O, S or N(Rₘ)-alkynyl; optionally substituted O-alkylenyl-O-alkyl, optionally substituted alkynyl, optionally substituted alkaryl, optionally substituted aralkyl, optionally substituted O-alkaryl, optionally substituted O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl; wherein each optionally substituted group is optionally substituted with a substituent group independently selected from among: hydroxyl, amino, alkoxy, carboxy, benzyl, phenyl, nitro (NO₂), thiol, thioalkoxy (S-alkyl), halogen, alkyl, aryl, alkenyl and alkynyl.
Embodiment 140: The oligomeric compound of embodiment 139, wherein each W nucleoside comprises a 2′-substituted sugar moiety comprising a 2′-substituent selected from among: a halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂.
Embodiment 141: The oligomeric compound of embodiment 139, wherein each W nucleoside comprises a 2′-substituted sugar moiety comprising a 2′-substituent selected from among: F, OCH₃, O(CH₂)₂—OCH₃.
Embodiment 142: The oligomeric compound of any of embodiments 102-137, wherein each W nucleoside comprises a bicyclic sugar moiety.
Embodiment 143: The oligomeric compound of embodiment 142, wherein each W nucleoside comprises a bicyclic sugar moiety selected from among: cEt, cMOE, LNA, α-LNA, ENA and 2′-thio LNA.
Embodiment 144: The oligomeric compound of any of embodiments 102-137, wherein each W comprises a modified nucleobase.
Embodiment 145: The oligomeric compound of embodiment 144, wherein each W comprises a modified nucleobase selected from among a 2-thio pyrimidine and a 5-propyne pyrimidine.
Embodiment 146: The oligomeric compound of embodiment 145, wherein each W comprises 2-thio-thymidine.
Embodiment 147: The oligomeric compound of embodiment 102-137, wherein each W comprises an F-HNA sugar moiety.
Embodiment 148: The oligomeric compound of embodiment 102-137, wherein each W nucleoside comprises an unmodified 2′-deoxyfuranose sugar moiety.
Embodiment 149: The oligomeric compound of any of embodiments 1-148, wherein the 3′ region consists of 2 linked 3′-region nucleosides.
Embodiment 150: The oligomeric compound of any of embodiments 1-148, wherein the 3′ region consists of 3 linked 3′-region nucleosides.
Embodiment 151: The oligomeric compound of any of embodiments 1-148, wherein the 3′ region consists of 4 linked 3′-region nucleosides.
Embodiment 152: The oligomeric compound of any of embodiments 1-148, wherein the 3′ region consists of 5 linked 3′-region nucleosides.
Embodiment 153: The oligomeric compound of any of embodiments 1-148, wherein the 3′ region consists of 6 linked 3′-region nucleosides.
Embodiment 154: The oligomeric compound of any of embodiments 1-153, wherein at least one 3′-region nucleoside is an unmodified deoxynucleoside.
Embodiment 155: The oligomeric compound of any of embodiments 1-154, wherein each 3′-region nucleoside is a modified nucleoside.
Embodiment 156: The oligomeric compound of any of embodiments 1-153, wherein at least one 3′-region nucleoside is an RNA-like nucleoside.
Embodiment 157: The oligomeric compound of any of embodiments 1-154, wherein each 3′-region nucleoside is an RNA-like nucleoside.
Embodiment 158: The oligomeric compound of any of embodiments 1-153, comprising at least one modified 3′-region nucleoside comprising a modified sugar.
Embodiment 159: The oligomeric compound of embodiment 158, comprising at least one modified 3′-region nucleoside comprising a bicyclic sugar moiety.
Embodiment 160: The oligomeric compound of embodiment 159, comprising at least one modified 3′-region nucleoside comprising a cEt sugar moiety.
Embodiment 161: The oligomeric compound of embodiment 159, comprising at least one modified 3′-region nucleoside comprising an LNA sugar moiety.
Embodiment 162: The oligomeric compound of any of embodiments 1-162 comprising of at least one modified 3′-region nucleoside comprising a 2′-substituted sugar moiety.
Embodiment 163: The oligomeric compound of embodiment 162, wherein at least one central region nucleoside comprises a 2′-substituted sugar moiety comprising a 2′ substituent selected from among: halogen, optionally substituted allyl, optionally substituted amino, azido, optionally substituted SH, CN, OCN, CF₃, OCF₃, O, S, or N(Rₘ)-alkyl; O, S, or N(Rₘ)-alkenyl; O, S or N(Rₘ)-alkynyl; optionally substituted O-alkylenyl-O-alkyl, optionally substituted alkynyl, optionally substituted alkaryl, optionally substituted aralkyl, optionally substituted O-alkaryl, optionally substituted O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl; wherein each optionally substituted group is optionally substituted with a substituent group independently selected from among: hydroxyl, amino, alkoxy, carboxy, benzyl, phenyl, nitro (NO₂), thiol, thioalkoxy (S-alkyl), halogen, alkyl, aryl, alkenyl and alkynyl.
Embodiment 164: The oligomeric compound of embodiment 163 wherein at least one modified 3′-region nucleoside comprises a 2′-substituted sugar moiety comprising a 2′-substituent selected from among: a halogen, OCH₃, OCH₂F, OCHF₂, OCF₃, OCH₂CH₃, O(CH₂)₂F, OCH₂CHF₂, OCH₂CF₃, OCH₂—CH═CH₂, O(CH₂)₂—OCH₃ (MOE), O(CH₂)₂—SCH₃, O(CH₂)₂—OCF₃, O(CH₂)₃—N(R₁)(R₂), O(CH₂)₂—ON(R₁)(R₂), O(CH₂)₂—O(CH₂)₂—N(R₁)(R₂), OCH₂C(═O)—N(R₁)(R₂), OCH₂C(═O)—N(R₃)—(CH₂)₂—N(R₁)(R₂), and O(CH₂)₂—N(R₃)—C(═NR₄)[N(R₁)(R₂)]; wherein R₁, R₂, R₃ and R₄ are each, independently, H or C₁-C₆ alkyl.
Embodiment 165: The oligomeric compound of embodiment 164, wherein the 2′-substituent is selected from among: a halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂.
Embodiment 166: The oligomeric compound of any of embodiments 162-165 comprising at least one modified 3′-region nucleoside comprising a 2′-MOE sugar moiety.
Embodiment 167: The oligomeric compound of any of embodiments 162-166 comprising at least one modified 3′-region nucleoside comprising a 2′-OMe sugar moiety.
Embodiment 168: The oligomeric compound of any of embodiments 162-167 comprising at least one modified 3′-region nucleoside comprising a 2′-F sugar moiety.
Embodiment 169: The oligomeric compound of any of embodiments 162-168 comprising at least one modified 3′-region nucleoside comprising a 2′-(ara)-F sugar moiety.
Embodiment 170: The oligomeric compound of any of embodiments 162-169 comprising of at least one modified 3′-region nucleoside comprising a sugar surrogate.
Embodiment 171: The oligomeric compound of embodiment 170 comprising at least one modified 3′-region nucleoside comprising an F-HNA sugar moiety.
Embodiment 172: The oligomeric compound of embodiment 170 comprising at least one modified 3′-region nucleoside comprising an HNA sugar moiety.
Embodiment 173: The oligomeric compound of any of embodiments 1-172 comprising at least one modified 3′-region nucleoside comprising a modified nucleobase.
Embodiment 174: The oligomeric compound of any of embodiments 1-173, wherein each A comprises a 2′-substituted sugar moiety comprising a 2′-substituent selected from among: F, OCH₃, O(CH₂)₂—OCH₃, and each B comprises a bicyclic sugar moiety selected from among: LNA and cEt.
Embodiment 175: The oligomeric compound of embodiment 174, wherein each A comprises O(CH₂)₂—OCH₃ and each B comprises cEt.
Embodiment 176: The oligomeric compound of any of embodiments 1-175, wherein the 3′-region has a motif selected from among: ABB, ABAA, AAABAA, AAAAABAA, AABAA, AAAABAA, AAABAA, ABAB, AAAAA, AAABB, AAAAAAAA, AAAAAAA, AAAAAA, AAAAB, AAAA, AAA, AA, AB, ABBB, ABAB, AABBB, wherein each A is a modified nucleoside of a first type, each B is a modified nucleoside of a second type.
Embodiment 177: The oligomeric compound of embodiments 1-175, wherein the 3′-region has a motif selected from among: ABB; AAABAA; AABAA; AAAABAA; AAAAA; AAABB; AAAAAAAA;
AAAAAAA; AAAAAA; AAAAB; AB; ABBB; and ABAB, wherein each A is a modified nucleoside of a first type, each B is a modified nucleoside of a second type.
Embodiment 178: The oligomeric compound of embodiments 1-175, wherein the 3′-region has a motif selected from among: BBA, AAB, AAA, BBB, BBAA, AABB, WBBA, WAAB, BBBA, BBBBA, BBBB, BBBBBA, ABBBBB, BBAAA, AABBB, BBBAA, BBBBA, BBBBB, BABA, AAAAA, BBAAAA, AABBBB, BAAAA, and ABBBB, wherein each A is a modified nucleoside of a first type, each B is a modified nucleoside of a second type, and each W is a modified nucleoside of a first type, a second type, or a third type.
Embodiment 179: The oligomeric compound of embodiments 176-178, wherein each A nucleoside comprises a 2′-substituted sugar moiety.
Embodiment 180: The oligomeric compound of embodiments 176-178, wherein at least one central region nucleoside comprises a 2′-substituted sugar moiety comprising a 2′ substituent selected from among: halogen, optionally substituted allyl, optionally substituted amino, azido, optionally substituted SH, CN, OCN, CF₃, OCF₃, O, S, or N(Rₘ)-alkyl; O, S, or N(Rₘ)-alkenyl; O, S or N(Rₘ)-alkynyl; optionally substituted O-alkylenyl-O-alkyl, optionally substituted alkynyl, optionally substituted alkaryl, optionally substituted aralkyl, optionally substituted O-alkaryl, optionally substituted O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl;
wherein each optionally substituted group is optionally substituted with a substituent group independently selected from among: hydroxyl, amino, alkoxy, carboxy, benzyl, phenyl, nitro (NO₂), thiol, thioalkoxy (S-alkyl), halogen, alkyl, aryl, alkenyl and alkynyl.
Embodiment 181: The oligomeric compound of embodiment 180, wherein each A nucleoside comprises a 2′-substituted sugar moiety comprising a 2′-substituent selected from among: a halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂.
Embodiment 182: The oligomeric compound of embodiment 181, wherein each A nucleoside comprises a 2′-substituted sugar moiety comprising a 2′-substituent selected from among: F, OCH₃, O(CH₂)₂—OCH₃.
Embodiment 183: The oligomeric compound of embodiments 176-178, wherein each A nucleoside comprises a bicyclic sugar moiety.
Embodiment 184: The oligomeric compound of embodiment 183, wherein each A nucleoside comprises a bicyclic sugar moiety selected from among: cEt, cMOE, LNA, α-LNA, ENA and 2′-thio LNA.
Embodiment 185: The oligomeric compound of any of embodiments 176-178, wherein each B nucleoside comprises a 2′-substituted sugar moiety.
Embodiment 186: The oligomeric compound of embodiment 185, wherein at least one modified central region nucleoside comprises a 2′-substituted sugar moiety comprising a 2′ substituent selected from among: halogen, optionally substituted allyl, optionally substituted amino, azido, optionally substituted SH, CN, OCN, CF₃, OCF₃, O, S, or N(Rₘ)-alkyl; O, S, or N(Rₘ)-alkenyl; O, S or N(Rₘ)-alkynyl; optionally substituted O-alkylenyl-O-alkyl, optionally substituted alkynyl, optionally substituted alkaryl, optionally substituted aralkyl, optionally substituted O-alkaryl, optionally substituted O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl;
wherein each optionally substituted group is optionally substituted with a substituent group independently selected from among: hydroxyl, amino, alkoxy, carboxy, benzyl, phenyl, nitro (NO₂), thiol, thioalkoxy (S-alkyl), halogen, alkyl, aryl, alkenyl and alkynyl.
Embodiment 187: The oligomeric compound of embodiment 185, wherein each B nucleoside comprises a 2′-substituted sugar moiety comprising a 2′-substituent selected from among: a halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂.
Embodiment 188: The oligomeric compound of embodiment 187, wherein each B nucleoside comprises a 2′-substituted sugar moiety comprising a 2′-substituent selected from among: F, OCH₃, O(CH₂)₂—OCH₃.
Embodiment 189: The oligomeric compound of any of embodiments 176-178, wherein each B nucleoside comprises a bicyclic sugar moiety.
Embodiment 190: The oligomeric compound of embodiment 189, wherein each B nucleoside comprises a bicyclic sugar moiety selected from among: cEt, cMOE, LNA, α-LNA, ENA and 2′-thio LNA.
Embodiment 191: The oligomeric compound of any of embodiments 176-190, wherein each A comprises a 2′-substituted sugar moiety comprising a 2′-substituent selected from among: F, OCH₃, O(CH₂)₂—OCH₃, and each B comprises a bicyclic sugar moiety selected from among: LNA and cEt.
Embodiment 192: The oligomeric compound of embodiment 191, wherein each A comprises O(CH₂)₂—OCH₃ and each B comprises cEt.
Embodiment 193: The oligomeric compound of any of embodiments 176-192, wherein each W nucleoside comprises a 2′-substituted sugar moiety.
Embodiment 194: The oligomeric compound of embodiment 193, wherein at least one central region nucleoside comprises a 2′-substituted sugar moiety comprising a 2′ substituent selected from among: halogen, optionally substituted allyl, optionally substituted amino, azido, optionally substituted SH, CN, OCN, CF₃, OCF₃, O, S, or N(Rₘ)-alkyl; O, S, or N(Rₘ)-alkenyl; O, S or N(Rₘ)-alkynyl; optionally substituted O-alkylenyl-O-alkyl, optionally substituted alkynyl, optionally substituted alkaryl, optionally substituted aralkyl, optionally substituted O-alkaryl, optionally substituted O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl; wherein each optionally substituted group is optionally substituted with a substituent group independently selected from among: hydroxyl, amino, alkoxy, carboxy, benzyl, phenyl, nitro (NO₂), thiol, thioalkoxy (S-alkyl), halogen, alkyl, aryl, alkenyl and alkynyl.
Embodiment 195: The oligomeric compound of embodiment 193, wherein each W nucleoside comprises a 2′-substituted sugar moiety comprising a 2′-substituent selected from among: a halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂.
Embodiment 196: The oligomeric compound of embodiment 195, wherein each W nucleoside comprises a 2′-substituted sugar moiety comprising a 2′-substituent selected from among: F, OCH₃, O(CH₂)₂—OCH₃.
Embodiment 197: The oligomeric compound of any of embodiments 176-192, wherein each W nucleoside comprises a bicyclic sugar moiety.
Embodiment 198: The oligomeric compound of embodiment 197, wherein each W nucleoside comprises a bicyclic sugar moiety selected from among: cEt, cMOE, LNA, α-LNA, ENA and 2′-thio LNA.
Embodiment 199: The oligomeric compound of any of embodiments 176-192, wherein each W comprises a modified nucleobase.
Embodiment 200: The oligomeric compound of embodiment 199, wherein each W comprises a modified nucleobase selected from among a 2-thio pyrimidine and a 5-propyne pyrimidine.
Embodiment 201: The oligomeric compound of embodiment 200, wherein each W comprises 2-thio-thymidine.
Embodiment 202: The oligomeric compound of embodiment 176-192, wherein each W comprises an F-HNA sugar moiety.
Embodiment 203: The oligomeric compound of embodiment 202, wherein each W nucleoside comprises an unmodified 2′-deoxyfuranose sugar moiety.
Embodiment 204: The oligomeric compound of embodiments 1-203, wherein the 5′-region has a motif selected from among: AB, ABB, AAA, BBB, BBBAA, AAB, BAA, BBAA, AABB, AAAB, ABBW, ABBWW, ABBB, ABBBB, ABAB, ABABAB, ABABBB, ABABAA, AAABB, AAAABB, AABB, AAAAB, AABBB, ABBBB, BBBBB, AAABW, AAAAA, and BBBBAA;
wherein the 3′-region has a motif selected from among: BBA, AAB, AAA, BBB, BBAA, AABB, WBBA, WAAB, BBBA, BBBBA, BBBB, BBBBBA, ABBBBB, BBAAA, AABBB, BBBAA, BBBBA, BBBBB, BABA, AAAAA, BBAAAA, AABBBB, BAAAA, and ABBBB;
wherein the central region has a nucleoside motif selected from among: DDDDDD, DDDDDDD, DDDDDDDD, DDDDDDDDD, DDDDDDDDDD, DXDDDDDDD, DDXDDDDDD, DDDXDDDDD, DDDDXDDDD, DDDDDXDDD, DDDDDDXDD, DDDDDDDXD, DXXDDDDDD, DDDDDDXXD, DDXXDDDDD, DDDXXDDDD, DDDDXXDDD, DDDDDXXDD, DXDDDDDXD, DXDDDDXDD, DXDDDXDDD, DXDDXDDDD, DXDXDDDDD, DDXDDDDXD, DDXDDDXDD, DDXDDXDDD, DDXDXDDDD, DDDXDDDXD, DDDXDDXDD, DDDXDXDDD, DDDDXDDXD, DDDDXDXDD, and DDDDDXDXD, DDDDDDDD, DXDDDDDD, DDXDDDDD, DDDXDDDD, DDDDXDDD, DDDDDXDD, DDDDDDXD, DXDDDDXD, DXDDDXDD, DXDDXDDD, DXDXDDDD, DXXDDDDD, DDXXDDDD, DDXDXDDD, DDXDDXDD, DXDDDDXD, DDDXXDDD, DDDXDXDD, DDDXDDXD, DDDDXXDD, DDDDXDXD, and DDDDDXXD, DXDDDDD, DDXDDDD, DDDXDDD, DDDDXDD, DDDDDXD, DXDDDXD, DXDDXDD, DXDXDDD, DXXDDDD, DDXXDDD, DDXDXDD, DDXDDXD, DDDXXDD, DDDXDXD, and DDDDXXD, DXDDDD, DDXDDD, DDDXDD, DDDDXD, DXXDDD, DXDXDD, DXDDXD, DDXXDD, DDXDXD, and DDDXXD; and wherein each A is a modified nucleoside of a first type, each B is a modified nucleoside of a second type, each W is a modified nucleoside of a first type, a second type, or a third type, each D is an unmodified deoxynucleoside, and each X is a modified nucleoside or a modified nucleobase.
Embodiment 205: The oligomeric compound of embodiment 204, wherein the 5′-region has a motif selected from among: AB, ABB, AAA, BBB, BBBAA, AAB, BAA, BBAA, AABB, ABBW, ABBWW, ABBB, ABBBB, ABAB, ABABAB, ABABBB, ABABAA, AAABB, AAAABB, AABB, AAAAB, AABBB, ABBBB, BBBBB, AAABW, and BBBBAA; and wherein the 3′-region has a BBA motif.
Embodiment 206: The oligomeric compound of embodiment 204 or 205, wherein one of A or B comprises a bicyclic sugar moiety, another of A or B comprises a 2′-MOE sugar moiety, and W comprises a 2-thio-thymidine nucleobase.
Embodiment 207: The oligomeric compound of embodiment 204 or 205, wherein one of A or B comprises a bicyclic sugar moiety, another of A or B comprises a 2′-MOE sugar moiety, and W comprises FHNA.
Embodiment 208: The oligomeric compound of embodiment 204 or 205, wherein one of A or B comprises cEt, another of A or B comprises a 2′-modified sugar moiety, and W comprises a 2-thio-thymidine nucleobase.
Embodiment 209: The oligomeric compound of embodiment 204 or 205, wherein one of A or B comprises cEt, another of A or B comprises a 2′-modified sugar moiety, and W comprises FHNA.
Embodiment 210: The oligomeric compound of embodiment 204 or 205, wherein each A comprises MOE, each B comprises cEt, and each W is selected from among cEt or FHNA.
Embodiment 211: The oligomeric compound of embodiment 204 or 205, wherein each W comprises cEt.
Embodiment 212: The oligomeric compound of embodiment 204 or 205, wherein each W comprises 2-thio-thymidine.
Embodiment 213: The oligomeric compound of embodiment 204 or 205, wherein each W comprises FHNA.
Embodiment 214: The oligomeric compound of any of embodiments 1-213 comprising at least one modified internucleoside linkage.
Embodiment 215: The oligomeric compound of embodiment 214, wherein each internucleoside linkage is a modified internucleoside linkage.
Embodiment 216: The oligomeric compound of embodiment 214 or 215 comprising at least one phosphorothioate internucleoside linkage.
Embodiment 217: The oligomeric compound of any of embodiments 214 or 215 comprising at least one methylphosphonate internucleoside linkage.
Embodiment 218: The oligomeric compound of any of embodiments 214 or 215 comprising one methylphosphonate internucleoside linkage.
Embodiment 219: The oligomeric compound of any of embodiments 214 or 215 comprising two methylphosphonate internucleoside linkages.
Embodiment 220: The oligomeric compound of embodiment 217, wherein at least one of the 3^{rd}, 4^{th}, 5^{th}, 6^{th} and/or 7^{th} internucleoside from the 5′-end is a methylphosphonate internucleoside linkage.
Embodiment 221: The oligomeric compound of embodiment 217, wherein at least one of the 3^{rd}, 4^{th}, 5^{th}, 6^{th} and/or 7^{th} internucleoside from the 3′-end is a methylphosphonate internucleoside linkage.
Embodiment 222: The oligomeric compound of embodiment 217, wherein at least one of the 3^{rd}, 4^{th}, 5^{th}, 6^{th}, 7^{th}, 8^{th}, 9^{th}, 10^{th}, 11^{th}, and/or 12^{th} internucleoside from the 5′-end is a methylphosphonate internucleoside linkage, and wherein at least one of the 3^{rd}, 4^{th}, 5^{th}, 6^{th}, 7^{th}, 8^{th}, 9^{th}, 10^{th}, 11^{th}, and/or 12^{th} internucleoside from the 5′-end is a modified nucleoside.
Embodiment 223: The oligomeric compound of embodiment 217, wherein at least one of the 3^{rd}, 4^{th}, 5^{th}, 6^{th}, 7^{th}, 8^{th}, 9^{th}, 10^{th}, 11^{th}, and/or 12^{th} internucleoside from the 3′-end is a methylphosphonate internucleoside linkage, and wherein at least one of the 3^{rd}, 4^{th}, 5^{th}, 6^{th}, 7^{th}, 8^{th}, 9^{th}, 10^{th}, 11^{th}, and/or 12^{th} internucleoside from the 3′-end is a modified nucleoside.
Embodiment 224: The oligomeric compound of any of embodiments 1-223 comprising at least one conjugate group.
Embodiment 225: The oligomeric compound of embodiment 1-223, wherein the conjugate group consists of a conjugate.
Embodiment 226: The oligomeric compound of embodiment 225, wherein the conjugate group consists of a conjugate and a conjugate linker.
Embodiment 227: The oligomeric compound of any of embodiments 1-226, wherein the nucleobase sequence of the modified oligonucleotide is 100% complementary to the nucleobase sequence of the target region of the target nucleic acid.
Embodiment 228: The oligomeric compound of any of embodiments 1-226, wherein the nucleobase sequence of the modified oligonucleotide contains one mismatch relative to the nucleobase sequence of the target region of the target nucleic acid.
Embodiment 229: The oligomeric compound of any of embodiments 1-226, wherein the nucleobase sequence of the modified oligonucleotide contains two mismatches relative to the nucleobase sequence of the target region of the target nucleic acid.
Embodiment 230: The oligomeric compound of any of embodiments 1-226, wherein the nucleobase sequence of the modified oligonucleotide comprises a hybridizing region and at least one non-targeting region, wherein the nucleobase sequence of the hybridizing region is complementary to the nucleobase sequence of the target region of the target nucleic acid.
Embodiment 231: The oligomeric compound of embodiment 230, wherein the nucleobase sequence of the hybridizing region is 100% complementary to the nucleobase sequence of the target region of the target nucleic acid.
Embodiment 232: The oligomeric compound of embodiment 230, wherein the nucleobase sequence of the hybridizing region contains one mismatche relative to the nucleobase sequence of the target region of the target nucleic acid.
Embodiment 233: The oligomeric compound of any of embodiments 230-232, wherein the nucleobase sequence of at least one non-targeting region is complementary to a portion of the hybridizing region of the modified oligonucleotide.
Embodiment 234: The oligomeric compound of embodiment 233, wherein the nucleobase sequence of at least one non-targeting region is 100% complementary to a portion of the hybridizing region of the modified oligonucleotide.
Embodiment 235: The oligomeric compound of embodiment 1-234 wherein the nucleobase sequence of the modified oligonucleotide comprises two non-targeting regions flanking a central hybridizing region.
Embodiment 236: The oligomeric compound of embodiment 235, wherein the two non-targeting regions are complementary to one another.
Embodiment 237: The oligomeric compound of embodiment 236, wherein the two non-targeting regions are 100% complementary to one another.
Embodiment 238: The oligomeric compound of any of embodiments 2-237, wherein the nucleobase sequence of the modified oligonucleotide aligns with the nucleobase of the target region of the target nucleic acid such that a distinguishing nucleobase of the target region of the target nucleic acid aligns with a target-selective nucleoside within the central region of the modified oligonucleotide.
Embodiment 239: The oligomeric compound of any of embodiments 3-237, wherein the nucleobase sequence of the modified oligonucleotide aligns with the nucleobase of the target region of the target nucleic acid such that the single distinguishing nucleobase of the target region of the target nucleic acid aligns with a target-selective nucleoside within the central region of the modified oligonucleotide.
Embodiment 240: The oligomeric compound of embodiment 238 or 239, wherein the target-selective nucleoside is the 5′-most nucleoside of the central region.
Embodiment 241: The oligomeric compound of embodiment 238 or 239, wherein the target-selective nucleoside is the 2^{nd} nucleoside from the 5′-end of the central region.
Embodiment 242: The oligomeric compound of embodiment 238 or 239, wherein the target-selective nucleoside is at the 3^{rd} nucleoside from the 5′-end of the central region.
Embodiment 243: The oligomeric compound of embodiment 238 or 239, wherein the target-selective nucleoside is at the 5^{th} nucleoside from the 5′-end of the central region.
Embodiment 244: The oligomeric compound of embodiment 238 or 239, wherein the target-selective nucleoside is at the 7^{th} nucleoside from the 5′-end of the central region.
Embodiment 245: The oligomeric compound of embodiment 238 or 239, wherein the target-selective nucleoside is at the 9^{th} nucleoside from the 5′-end of the central region.
Embodiment 246: The oligomeric compound of any of embodiments 238 or 239, or 241-245, wherein the target-selective nucleoside is at the 2^{nd} nucleoside from the 3′-end of the central region.
Embodiment 247: The oligomeric compound of any of embodiments 238 or 239, or 241-245, wherein the target-selective nucleoside is at the 5^{th} nucleoside from the 3′-end of the central region.
Embodiment 248: The oligomeric compound of any of embodiments 1-247, wherein target-selective nucleoside is an unmodified deoxynucleoside.
Embodiment 249: The oligomeric compound of any of embodiments 1-247, wherein target-selective nucleoside is a modified nucleoside.
Embodiment 250: The oligomeric compound of embodiment 249, wherein the target-selective nucleoside is a sugar modified nucleoside.
Embodiment 251: The oligomeric compound of embodiment 250, wherein the target-selective nucleoside comprises a sugar modification selected from among: 2′-MOE, 2′-F, 2′-(ara)-F, HNA, FHNA, cEt, and α-L-LNA.
Embodiment 252: The oligomeric compound of any of embodiments 1-251, wherein the target-selective nucleoside comprises a nucleobase modification.
Embodiment 253: The oligomeric compound of embodiment 252, wherein the modified nucleobase is selected from among: a 2-thio pyrimidine and a 5-propyne pyrimidine.
Embodiment 254: The oligomeric compound of any of embodiments 1-253, wherein the oligomeric compound is an antisense compound.
Embodiment 255: The oligomeric compound of embodiment 254, wherein the oligomeric compound selectively reduces expression of the target relative to the non-target.
Embodiment 256: The oligomeric compound of embodiment 255, wherein the oligomeric compound reduces expression of target at least two-fold more than it reduces expression of the non-target.
Embodiment 257: The oligomeric compound of embodiment 256, having an EC₅₀ for reduction of expression of target that is at least two-fold lower than its EC₅₀ for reduction of expression of the non-target, when measured in cells.
Embodiment 258: The oligomeric compound of embodiment 256, having an ED₅₀ for reduction of expression of target that is at least two-fold lower than its ED₅₀ for reduction of expression of the non-target, when measured in an animal.
Embodiment 259: The oligomeric compound of embodiments 1-10, having an E-E-E-K-K-(D)₇-E-E-K motif, wherein each E is a 2′-MOE nucleoside and each K is a cEt nucleoside.
Embodiment 260: A method comprising contacting a cell with an oligomeric compound of any of embodiments 1-259.
Embodiment 261: The method of embodiment 260, wherein the cell is in vitro.
Embodiment 262: The method of embodiment 260, wherein the cell is in an animal.
Embodiment 263: The method of embodiment 262, wherein the animal is a human.
Embodiment 264: The method of embodiment 263, wherein the animal is a mouse.
Embodiment 265: A pharmaceutical composition comprising an oligomeric compound of any of embodiments 1-259 and a pharmaceutically acceptable carrier or diluent.
Embodiment 266: A method of administering a pharmaceutical composition of embodiment 265 to an animal.
Embodiment 267: The method of embodiment 266, wherein the animal is a human.
Embodiment 268: The method of embodiment 266, wherein the animal is a mouse.
Embodiment 269: Use of an oligomeric compound of any of embodiments 1-259 for the preparation of a medicament for the treatment or amelioration of Alzheimer's disease, Creutzfeldt-Jakob disease, fatal familial insomnia, Alexander disease, Parkinson's disease, amyotrophic lateral sclerosis, dentato-rubral and pallido-luysian atrophy DRPA, spino-cerebellar ataxia, Torsion dystonia, cardiomyopathy, chronic obstructive pulmonary disease (COPD), liver disease, hepatocellular carcinoma, systemic lupus erythematosus, hypercholesterolemia, breast cancer, asthma, Type 1 diabetes, Rheumatoid arthritis, Graves disease, SLE, spinal and bulbar muscular atrophy, Kennedy's disease, progressive childhood posterior subcapsular cataracts, cholesterol gallstone disease, arthrosclerosis, cardiovascular disease, primary hypercalciuria, alpha-thallasemia, obsessive compulsive disorder, Anxiety, comorbid depression, congenital visual defects, hypertension, metabolic syndrome, prostate cancer, congential myasthenic syndrome, peripheral arterial disease, atrial fibrillation, sporadic pheochromocytoma, congenital malformations, Machado-Joseph disease, Huntington's disease, and Autosomal Dominant Retinitis Pigmentosa disease.
Embodiment 270: A method of ameliorating a symptom of Alzheimer's disease, Creutzfeldt-Jakob disease, fatal familial insomnia, Alexander disease, Parkinson's disease, amyotrophic lateral sclerosis, dentato-rubral and pallido-luysian atrophy DRPA, spino-cerebellar ataxia, Torsion dystonia, cardiomyopathy, chronic obstructive pulmonary disease (COPD), liver disease, hepatocellular carcinoma, systemic lupus erythematosus, hypercholesterolemia, breast cancer, asthma, Type 1 diabetes, Rheumatoid arthritis, Graves disease, SLE, spinal and bulbar muscular atrophy, Kennedy's disease, progressive childhood posterior subcapsular cataracts, cholesterol gallstone disease, arthrosclerosis, cardiovascular disease, primary hypercalciuria, alpha-thallasemia, obsessive compulsive disorder, Anxiety, comorbid depression, congenital visual defects, hypertension, metabolic syndrome, prostate cancer, congential myasthenic syndrome, peripheral arterial disease, atrial fibrillation, sporadic pheochromocytoma, congenital malformations, Machado-Joseph disease, Huntington's disease, and Autosomal Dominant Retinitis Pigmentosa disease, comprising administering an oligomeric compound of any of embodiments 1-259 to an animal in need thereof.
Embodiment 271: The method of embodiment 270, wherein the animal is a human.
Embodiment 272: The method of embodiment 270, wherein the animal is a mouse.

In certain embodiments, including but not limited to any of the above numbered embodiments, oligomeric compounds including oligonucleotides described herein are capable of modulating expression of a target RNA. In certain embodiments, the target RNA is associated with a disease or disorder, or encodes a protein that is associated with a disease or disorder. In certain embodiments, the oligomeric compounds or oligonucleotides provided herein modulate the expression of function of such RNA to alleviate one or more symptom of the disease or disorder.

In certain embodiments, oligomeric compounds including oligonucleotides describe herein are useful in vitro. In certain embodiments such oligomeric compounds are used in diagnostics and/or for target validation experiments.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### A. Definitions

Unless specific definitions are provided, the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21^{st} edition, 2005; and “Antisense Drug Technology, Principles, Strategies, and Applications” Edited by Stanley T. Crooke, CRC Press, Boca Raton, Florida; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety.

As used herein, “chemical modification” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

As used herein, “furanosyl” means a structure comprising a 5-membered ring comprising four carbon atoms and one oxygen atom.

As used herein, “naturally occurring sugar moiety” means a ribofuranosyl as found in naturally occurring RNA or a deoxyribofuranosyl as found in naturally occurring DNA.

As used herein, “sugar moiety” means a naturally occurring sugar moiety or a modified sugar moiety of a nucleoside.

As used herein, “modified sugar moiety” means a substituted sugar moiety or a sugar surrogate.

As used herein, “substituted sugar moiety” means a furanosyl that is not a naturally occurring sugar moiety. Substituted sugar moieties include, but are not limited to furanosyls comprising substituents at the 2′-position, the 3′-position, the 5′-position and/or the 4′-position. Certain substituted sugar moieties are bicyclic sugar moieties.

As used herein, “2′-substituted sugar moiety” means a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring.

As used herein, “MOE” means —OCH₂CH₂OCH₃.

As used herein, “2′-F nucleoside” refers to a nucleoside comprising a sugar comprising fluorine at the 2′ position. Unless otherwise indicated, the fluorine in a 2′-F nucleoside is in the ribo position (replacing the OH of a natural ribose).

As used herein, “2′-(ara)-F” refers to a 2′-F substituted nucleoside, wherein the fluoro group is in the arabino position.

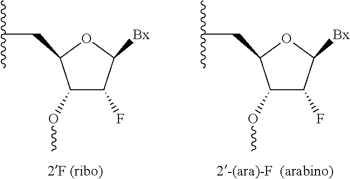

As used herein the term “sugar surrogate” means a structure that does not comprise a furanosyl and that is capable of replacing the naturally occurring sugar moiety of a nucleoside, such that the resulting nucleoside sub-units are capable of linking together and/or linking to other nucleosides to form an oligomeric compound which is capable of hybridizing to a complementary oligomeric compound. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

As used herein, “bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

As used herein, “nucleotide” means a nucleoside further comprising a phosphate linking group. As used herein, “linked nucleosides” may or may not be linked by phosphate linkages and thus includes, but is not limited to “linked nucleotides.” As used herein, “linked nucleosides” are nucleosides that are connected in a continuous sequence (i.e. no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “modified nucleoside” means a nucleoside comprising at least one chemical modification compared to naturally occurring RNA or DNA nucleosides. Modified nucleosides comprise a modified sugar moiety and/or a modified nucleobase.

As used herein, “bicyclic nucleoside” or “BNA” means a nucleoside comprising a bicyclic sugar moiety.

As used herein, “constrained ethyl nucleoside” or “cEt” means a nucleoside comprising a bicyclic sugar moiety comprising a 4′-CH(CH₃)—O-2′bridge.

As used herein, “locked nucleic acid nucleoside” or “LNA” means a nucleoside comprising a bicyclic sugar moiety comprising a 4′-CH₂—O-2′bridge.

As used herein, “2′-substituted nucleoside” means a nucleoside comprising a substituent at the 2′-position other than H or OH. Unless otherwise indicated, a 2′-substituted nucleoside is not a bicyclic nucleoside.

As used herein, “2′-deoxynucleoside” means a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

As used herein, “RNA-like nucleoside” means a modified nucleoside that adopts a northern configuration and functions like RNA when incorporated into an oligonucleotide. RNA-like nucleosides include, but are not limited to 3′-endo furanosyl nucleosides and RNA surrogates.

As used herein, “3′-endo-furanosyl nucleoside” means an RNA-like nucleoside that comprises a substituted sugar moiety that has a 3′-endo conformation. 3′-endo-furanosyl nucleosides include, but are not limited to: 2′-MOE, 2′-F, 2′-OMe, LNA, ENA, and cEt nucleosides.

As used herein, “RNA-surrogate nucleoside” means an RNA-like nucleoside that does not comprise a furanosyl. RNA-surrogate nucleosides include, but are not limited to hexitols and cyclopentanes.

As used herein, “oligonucleotide” means a compound comprising a plurality of linked nucleosides.

In certain embodiments, an oligonucleotide comprises one or more unmodified ribonucleosides (RNA) and/or unmodified deoxyribonucleosides (DNA) and/or one or more modified nucleosides.

As used herein “oligonucleoside” means an oligonucleotide in which none of the internucleoside linkages contains a phosphorus atom. As used herein, oligonucleotides include oligonucleosides.

As used herein, “modified oligonucleotide” means an oligonucleotide comprising at least one modified nucleoside and/or at least one modified internucleoside linkage.

As used herein “internucleoside linkage” means a covalent linkage between adjacent nucleosides in an oligonucleotide.

As used herein “naturally occurring internucleoside linkage” means a 3′ to 5′ phosphodiester linkage.

As used herein, “modified internucleoside linkage” means any internucleoside linkage other than a naturally occurring internucleoside linkage.

As used herein, “oligomeric compound” means a polymeric structure comprising two or more sub-structures. In certain embodiments, an oligomeric compound comprises an oligonucleotide. In certain embodiments, an oligomeric compound comprises one or more conjugate groups and/or terminal groups. In certain embodiments, an oligomeric compound consists of an oligonucleotide.

As used herein, “terminal group” means one or more atom attached to either, or both, the 3′ end or the 5′ end of an oligonucleotide. In certain embodiments a terminal group is a conjugate group. In certain embodiments, a terminal group comprises one or more terminal group nucleosides.

As used herein, “conjugate” means an atom or group of atoms bound to an oligonucleotide or oligomeric compound. In general, conjugate groups modify one or more properties of the compound to which they are attached, including, but not limited to pharmacodynamic, pharmacokinetic, binding, absorption, cellular distribution, cellular uptake, charge and/or clearance properties.

As used herein, “conjugate linking group” means any atom or group of atoms used to attach a conjugate to an oligonucleotide or oligomeric compound.

As used herein, “antisense compound” means a compound comprising or consisting of an oligonucleotide at least a portion of which is complementary to a target nucleic acid to which it is capable of hybridizing, resulting in at least one antisense activity.

As used herein, “antisense activity” means any detectable and/or measurable change attributable to the hybridization of an antisense compound to its target nucleic acid.

As used herein, “detecting” or “measuring” means that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

As used herein, “detectable and/or measureable activity” means a measurable activity that is not zero.

As used herein, “essentially unchanged” means little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

As used herein, “expression” means the process by which a gene ultimately results in a protein. Expression includes, but is not limited to, transcription, post-transcriptional modification (e.g., splicing, polyadenylation, addition of 5′-cap), and translation.

As used herein, “target nucleic acid” means a nucleic acid molecule to which an antisense compound is intended to hybridize.

As used herein, “non-target nucleic acid” means a nucleic acid molecule to which hybridization of an antisense compound is not intended or desired. In certain embodiments, antisense compounds do hybridize to a non-target, due to homology between the target (intended) and non-target (un-intended).

As used herein, “mRNA” means an RNA molecule that encodes a protein.

As used herein, “pre-mRNA” means an RNA transcript that has not been fully processed into mRNA. Pre-RNA includes one or more intron.

As used herein, “object RNA” means an RNA molecule other than a target RNA, the amount, activity, splicing, and/or function of which is modulated, either directly or indirectly, by a target nucleic acid.

In certain embodiments, a target nucleic acid modulates splicing of an object RNA. In certain such embodiments, an antisense compound modulates the amount or activity of the target nucleic acid, resulting in a change in the splicing of an object RNA and ultimately resulting in a change in the activity or function of the object RNA.

As used herein, “microRNA” means a naturally occurring, small, non-coding RNA that represses gene expression of at least one mRNA. In certain embodiments, a microRNA represses gene expression by binding to a target site within a 3′ untranslated region of an mRNA. In certain embodiments, a microRNA has a nucleobase sequence as set forth in miRBase, a database of published microRNA sequences found at http://microrna.sanger.ac.uk/sequences/. In certain embodiments, a microRNA has a nucleobase sequence as set forth in miRBase version 12.0 released September 2008, which is herein incorporated by reference in its entirety.

As used herein, “microRNA mimic” means an oligomeric compound having a sequence that is at least partially identical to that of a microRNA. In certain embodiments, a microRNA mimic comprises the microRNA seed region of a microRNA. In certain embodiments, a microRNA mimic modulates translation of more than one target nucleic acids. In certain embodiments, a microRNA mimic is double-stranded.

As used herein, “differentiating nucleobase” means a nucleobase that differs between two nucleic acids. In certain instances, a target region of a target nucleic acid differs by 1-4 nucleobases from a non-target nucleic acid. Each of those differences is referred to as a differentiating nucleobase. In certain instances, a differentiating nucleobase is a single-nucleotide polymorphism.

As used herein, “target-selective nucleoside” means a nucleoside of an antisense compound that corresponds to a differentiating nucleobase of a target nucleic acid.

As used herein, “allele” means one of a pair of copies of a gene existing at a particular locus or marker on a specific chromosome, or one member of a pair of nucleobases existing at a particular locus or marker on a specific chromosome, or one member of a pair of nucleobase sequences existing at a particular locus or marker on a specific chromosome. For a diploid organism or cell or for autosomal chromosomes, each allelic pair will normally occupy corresponding positions (loci) on a pair of homologous chromosomes, one inherited from the mother and one inherited from the father. If these alleles are identical, the organism or cell is said to be “homozygous” for that allele; if they differ, the organism or cell is said to be “heterozygous” for that allele. “Wild-type allele” refers to the genotype typically not associated with disease or dysfunction of the gene product. “Mutant allele” refers to the genotype associated with disease or dysfunction of the gene product.

As used herein, “allelic variant” means a particular identity of an allele, where more than one identity occurs. For example, an allelic variant may refer to either the mutant allele or the wild-type allele.

As used herein, “single nucleotide polymorphism” or “SNP” means a single nucleotide variation between the genomes of individuals of the same species. In some cases, a SNP may be a single nucleotide deletion or insertion. In general, SNPs occur relatively frequently in genomes and thus contribute to genetic diversity. The location of a SNP is generally flanked by highly conserved sequences. An individual may be homozygous or heterozygous for an allele at each SNP site.

As used herein, “single nucleotide polymorphism site” or “SNP site” refers to the nucleotides surrounding a SNP contained in a target nucleic acid to which an antisense compound is targeted.

As used herein, “targeting” or “targeted to” means the association of an antisense compound to a particular target nucleic acid molecule or a particular region of a target nucleic acid molecule. An antisense compound targets a target nucleic acid if it is sufficiently complementary to the target nucleic acid to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In certain embodiments, complementary nucleobase means a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides, or nucleic acids) means the capacity of such oligomeric compounds or regions thereof to hybridize to another oligomeric compound or region thereof through nucleobase complementarity under stringent conditions. Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80% complementary. In certain embodiments, complementary oligomeric compounds or regions are 90% complementary. In certain embodiments, complementary oligomeric compounds or regions are 95% complementary. In certain embodiments, complementary oligomeric compounds or regions are 100% complementary.

As used herein, “mismatch” means a nucleobase of a first oligomeric compound that is not capable of pairing with a nucleobase at a corresponding position of a second oligomeric compound, when the first and second oligomeric compound are aligned. Either or both of the first and second oligomeric compounds may be oligonucleotides.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site. In certain embodiments, an antisense oligonucleotide specifically hybridizes to more than one target site.

As used herein, “fully complementary” in reference to an oligonucleotide or portion thereof means that each nucleobase of the oligonucleotide or portion thereof is capable of pairing with a nucleobase of a complementary nucleic acid or contiguous portion thereof. Thus, a fully complementary region comprises no mismatches or unhybridized nucleobases in either strand.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression. As a further example, modulation of expression can include a change in splice site selection of pre-mRNA processing, resulting in a change in the absolute or relative amount of a particular splice-variant compared to the amount in the absence of modulation.

As used herein, “modification motif” means a pattern of chemical modifications in an oligomeric compound or a region thereof. Motifs may be defined by modifications at certain nucleosides and/or at certain linking groups of an oligomeric compound.

As used herein, “nucleoside motif” means a pattern of nucleoside modifications in an oligomeric compound or a region thereof. The linkages of such an oligomeric compound may be modified or unmodified. Unless otherwise indicated, motifs herein describing only nucleosides are intended to be nucleoside motifs. Thus, in such instances, the linkages are not limited.

As used herein, “sugar motif” means a pattern of sugar modifications in an oligomeric compound or a region thereof.

As used herein, “linkage motif” means a pattern of linkage modifications in an oligomeric compound or region thereof. The nucleosides of such an oligomeric compound may be modified or unmodified. Unless otherwise indicated, motifs herein describing only linkages are intended to be linkage motifs. Thus, in such instances, the nucleosides are not limited.

As used herein, “nucleobase modification motif” means a pattern of modifications to nucleobases along an oligonucleotide. Unless otherwise indicated, a nucleobase modification motif is independent of the nucleobase sequence.

As used herein, “sequence motif” means a pattern of nucleobases arranged along an oligonucleotide or portion thereof. Unless otherwise indicated, a sequence motif is independent of chemical modifications and thus may have any combination of chemical modifications, including no chemical modifications.

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, a MOE nucleoside and an unmodified DNA nucleoside are “differently modified,” even though the DNA nucleoside is unmodified.

Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

As used herein, “the same type of modifications” refers to modifications that are the same as one another, including absence of modifications. Thus, for example, two unmodified DNA nucleoside have “the same type of modification,” even though the DNA nucleoside is unmodified. Such nucleosides having the same type modification may comprise different nucleobases.

As used herein, “pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

As used herein, “substituent” and “substituent group,” means an atom or group that replaces the atom or group of a named parent compound. For example a substituent of a modified nucleoside is any atom or group that differs from the atom or group found in a naturally occurring nucleoside (e.g., a modified 2′-substituent is any atom or group at the 2′-position of a nucleoside other than H or OH). Substituent groups can be protected or unprotected. In certain embodiments, compounds of the present invention have substituents at one or at more than one position of the parent compound. Substituents may also be further substituted with other substituent groups and may be attached directly or via a linking group such as an alkyl or hydrocarbyl group to a parent compound.

Likewise, as used herein, “substituent” in reference to a chemical functional group means an atom or group of atoms differs from the atom or a group of atoms normally present in the named functional group. In certain embodiments, a substituent replaces a hydrogen atom of the functional group (e.g., in certain embodiments, the substituent of a substituted methyl group is an atom or group other than hydrogen which replaces one of the hydrogen atoms of an unsubstituted methyl group). Unless otherwise indicated, groups amenable for use as substituents include without limitation, halogen, hydroxyl, alkyl, alkenyl, alkynyl, acyl (—C(O)Rₐₐ), carboxyl (—C(O)O—Rₐₐ), aliphatic groups, alicyclic groups, alkoxy, substituted oxy (—O—Rₐₐ), aryl, aralkyl, heterocyclic radical, heteroaryl, heteroarylalkyl, amino (—N(R_{bb})(R_{cc})), imino (═NR_{bb}), amido (—C(O)N(R_{bb})(R_{cc}) or —N(R_{bb})C(O)Rₐₐ), azido (—N₃), nitro (—NO₂), cyano (—CN), carbamido (—OC(O)N(R_{bb})(R_{cc}) or —N(R_{bb})C(O)ORₐₐ), ureido (—N(R_{bb})C(O)N(R_{bb})(R_{cc})), thioureido (—N(R_{bb})C(S)N(R_{bb})—(R_{c})), guanidinyl (—N(R_{bb})C(═NR_{bb})N(R_{bb})(R_{cc})), amidinyl (—C(═NR_{bb})N(R_{bb})(R_{cc}) or —N(R_{bb})C(═NR_{bb})(Rₐₐ)), thiol (—SR_{bb}), sulfinyl (—S(O)R_{bb}), sulfonyl (—S(O)₂R_{bb}) and sulfonamidyl (—S(O)₂N(R_{bb})(R_{cc}) or —N(R_{bb})S—(O)₂R_{bb}). Wherein each Rₐₐ, R_{bb} and R_{cc} is, independently, H, an optionally linked chemical functional group or a further substituent group with a preferred list including without limitation, alkyl, alkenyl, alkynyl, aliphatic, alkoxy, acyl, aryl, aralkyl, heteroaryl, alicyclic, heterocyclic and heteroarylalkyl. Selected substituents within the compounds described herein are present to a recursive degree.

As used herein, “alkyl,” as used herein, means a saturated straight or branched hydrocarbon radical containing up to twenty four carbon atoms. Examples of alkyl groups include without limitation, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C₁-C₁₂ alkyl) with from 1 to about 6 carbon atoms being more preferred.

As used herein, “alkenyl,” means a straight or branched hydrocarbon chain radical containing up to twenty four carbon atoms and having at least one carbon-carbon double bond. Examples of alkenyl groups include without limitation, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

As used herein, “alkynyl,” means a straight or branched hydrocarbon radical containing up to twenty four carbon atoms and having at least one carbon-carbon triple bond. Examples of alkynyl groups include, without limitation, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substituent groups.

As used herein, “acyl,” means a radical formed by removal of a hydroxyl group from an organic acid and has the general Formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substituent groups.

As used herein, “alicyclic” means a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substituent groups.

As used herein, “aliphatic” means a straight or branched hydrocarbon radical containing up to twenty four carbon atoms wherein the saturation between any two carbon atoms is a single, double or triple bond.

An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation, polyalkoxys, such as polyalkylene glycols, polyamines, and polyimines. Aliphatic groups as used herein may optionally include further substituent groups.

As used herein, “alkoxy” means a radical formed between an alkyl group and an oxygen atom wherein the oxygen atom is used to attach the alkoxy group to a parent molecule. Examples of alkoxy groups include without limitation, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

As used herein, “aminoalkyl” means an amino substituted C₁-C₁₂alkyl radical. The alkyl portion of the radical forms a covalent bond with a parent molecule. The amino group can be located at any position and the aminoalkyl group can be substituted with a further substituent group at the alkyl and/or amino portions.

As used herein, “aralkyl” and “arylalkyl” mean an aromatic group that is covalently linked to a C₁-C₁₂ alkyl radical. The alkyl radical portion of the resulting aralkyl (or arylalkyl) group forms a covalent bond with a parent molecule. Examples include without limitation, benzyl, phenethyl and the like. Aralkyl groups as used herein may optionally include further substituent groups attached to the alkyl, the aryl or both groups that form the radical group.

As used herein, “aryl” and “aromatic” mean a mono- or polycyclic carbocyclic ring system radicals having one or more aromatic rings. Examples of aryl groups include without limitation, phenyl, naphthyl, tetrahydronaphthyl, indanyl, idenyl and the like. Preferred aryl ring systems have from about 5 to about 20 carbon atoms in one or more rings. Aryl groups as used herein may optionally include further substituent groups.

As used herein, “halo” and “halogen,” mean an atom selected from fluorine, chlorine, bromine and iodine.

As used herein, “heteroaryl,” and “heteroaromatic,” mean a radical comprising a mono- or polycyclic aromatic ring, ring system or fused ring system wherein at least one of the rings is aromatic and includes one or more heteroatoms. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl and the like. Heteroaryl radicals can be attached to a parent molecule directly or through a linking moiety such as an aliphatic group or hetero atom. Heteroaryl groups as used herein may optionally include further substituent groups.

### B. Oligomeric Compounds

In certain embodiments, the present invention provides oligomeric compounds. In certain embodiments, such oligomeric compounds comprise oligonucleotides optionally comprising one or more conjugate and/or terminal groups. In certain embodiments, an oligomeric compound consists of an oligonucleotide. In certain embodiments, oligonucleotides comprise one or more chemical modifications. Such chemical modifications include modifications of one or more nucleoside (including modifications to the sugar moiety and/or the nucleobase) and/or modifications to one or more internucleoside linkage.

a. Certain Modified Nucleosides

In certain embodiments, provided herein are oligomeric compounds comprising or consisting of oligonucleotides comprising at least one modified nucleoside. Such modified nucleosides comprise a modified sugar moeity, a modified nucleobase, or both a modified sugar moiety and a modified nucleobase.

i. Certain Modified Sugar Moieties

In certain embodiments, compounds of the invention comprise one or more modified nucleosides comprising a modified sugar moiety. Such compounds comprising one or more sugar-modified nucleosides may have desirable properties, such as enhanced nuclease stability or increased binding affinity with a target nucleic acid relative to an oligonucleotide comprising only nucleosides comprising naturally occurring sugar moieties. In certain embodiments, modified sugar moieties are substituted sugar moieties. In certain embodiments, modified sugar moieties are sugar surrogates. Such sugar surrogates may comprise one or more substitutions corresponding to those of substituted sugar moieties.

In certain embodiments, modified sugar moieties are substituted sugar moieties comprising one or more non-bridging sugar substituent, including but not limited to substituents at the 2′ and/or 5′ positions.

Examples of sugar substituents suitable for the 2′-position, include, but are not limited to: 2′-F, 2′-OCH₃ (“OMe” or “O-methyl”), and 2′-O(CH₂)₂OCH₃ (“MOE”). In certain embodiments, sugar substituents at the 2′ position is selected from allyl, amino, azido, thio, O-allyl, O—C₁-C₁₀ alkyl, O—C₁-C₁₀ substituted alkyl; OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In certain embodiments, substituted sugars comprise more than one non-bridging sugar substituent, for example, 2′-F-5′-methyl sugar moieties (see, e.g., PCT International Application WO 2008/101157, for additional 5′, 2′-bis substituted sugar moieties and nucleosides).

Nucleosides comprising 2′-substituted sugar moieties are referred to as 2′-substituted nucleosides. In certain embodiments, a 2′-substituted nucleoside comprises a 2′-substituent group selected from halo, allyl, amino, azido, SH, CN, OCN, CF₃, OCF₃, O, S, or N(Rₘ)-alkyl; O, S, or N(Rₘ)-alkenyl; 0, S or N(Rₘ)-alkynyl; O-alkylenyl-O-alkyl, alkynyl, alkaryl, aralkyl, O-alkaryl, O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In certain embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In certain embodiments, a 2′-substituted nucleoside comprises a sugar moiety comprising a 2′-substituent group selected from F, OCF₃, O—CH₃, OCH₂CH₂OCH₃, O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

In certain embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ—, —[C(Rₐ)(R_{b})]ₙ—O—, —C(RₐR_{b})—N(R)—O— or, —C(RₐR_{b})—O—N(R)—; 4′- CH₂-2′, 4′-(CH₂)₂-2′, 4′-(CH₂)₃-2′, 4′-(CH₂)—O-2′ (LNA); 4′-(CH₂)—S-2′; 4′-(CH₂)₂—O-2′ (ENA); 4′-CH(CH₃)—O-2′ (cEt) and 4′-CH(CH₂OCH₃)—O-2′, and analogs thereof (see, e.g., U.S. Pat. No. 7,399,845, issued on Jul. 15, 2008); 4′-C(CH₃)(CH₃)—O-2′ and analogs thereof, (see, e.g., WO2009/006478, published Jan. 8, 2009); 4′-CH₂—N(OCH₃)-2′ and analogs thereof (see, e.g., WO2008/150729, published Dec. 11, 2008); 4′-CH₂—O—N(CH₃)-2′ (see, e.g., US2004/0171570, published Sep. 2, 2004); 4′-CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group (see, U.S. Pat. No. 7,427,672, issued on Sep. 23, 2008); 4′-CH₂—C(H)(CH₃)-2′ (see, e.g., Chattopadhyaya, et al., J. Org. Chem., 2009, 74, 118-134); and 4′-CH₂—C(═CH₂)-2′ and analogs thereof (see, published PCT International Application WO 2008/154401, published on Dec. 8, 2008).

In certain embodiments, such 4′ to 2′ bridges independently comprise from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂—O-2′) BNA, (B) R-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃-2′) BNA, and (K) Ethylene(methoxy) (4′-(CH(CH₂OMe)-O-2′) BNA (also referred to as constrained MOE or cMOE) as depicted below.

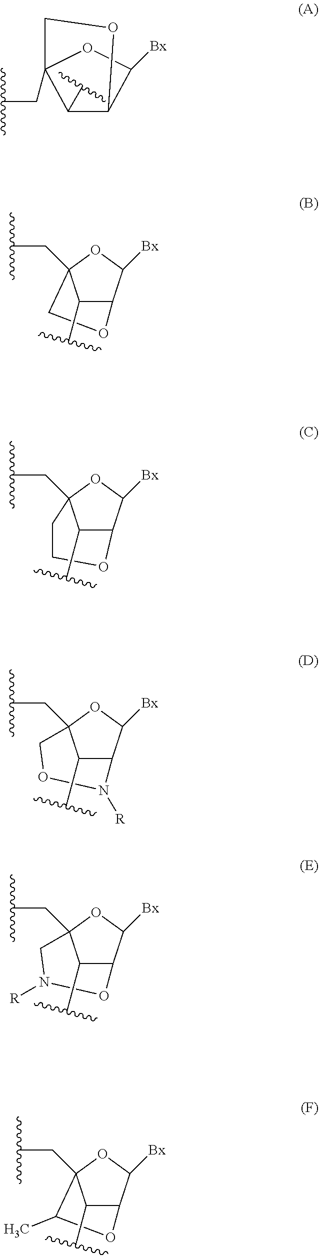

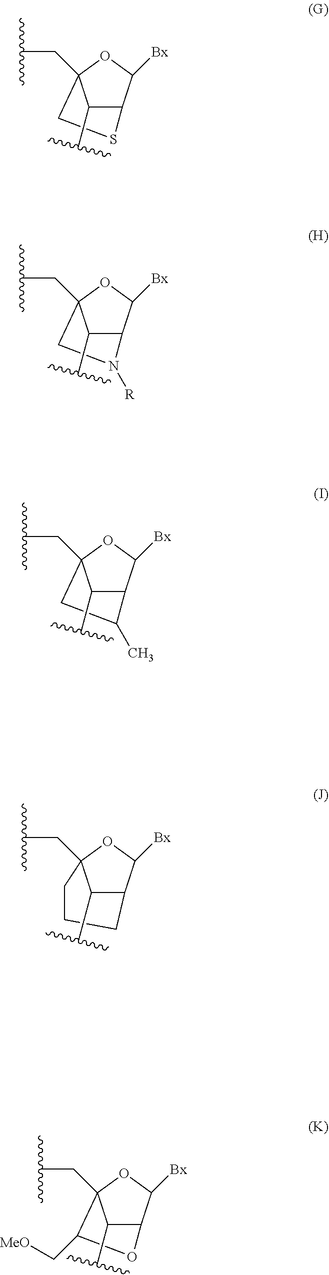

wherein Bx is a nucleobase moiety and R is, independently, H, a protecting group, or C₁-C₁₂ alkyl.

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US2004/0171570, US2007/0287831, and US2008/0039618; U.S. patent Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In certain embodiments, bicyclic sugar moieties and nucleosides incorporating such bicyclic sugar moieties are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) bicyclic nucleosides have been incorporated into antisense oligonucleotides that showed antisense activity (Frieden et al., Nucleic Acids Research, 2003, 21, 6365-6372).

In certain embodiments, substituted sugar moieties comprise one or more non-bridging sugar substituent and one or more bridging sugar substituent (e.g., 5′-substituted and 4′-2′ bridged sugars). (see, PCT International Application WO 2007/134181, published on Nov. 22, 2007, wherein LNA is substituted with, for example, a 5′-methyl or a 5′-vinyl group).

In certain embodiments, modified sugar moieties are sugar surrogates. In certain such embodiments, the oxygen atom of the naturally occurring sugar is substituted, e.g., with a sulfur, carbon or nitrogen atom. In certain such embodiments, such modified sugar moiety also comprises bridging and/or non-bridging substituents as described above. For example, certain sugar surrogates comprise a 4′-sulfur atom and a substitution at the 2′-position (see, e.g., published U.S. Patent Application US2005/0130923, published on Jun. 16, 2005) and/or the 5′ position. By way of additional example, carbocyclic bicyclic nucleosides having a 4-2′ bridge have been described (see, e.g., Freier et al., Nucleic Acids Research, 1997, 25(22), 4429-4443 and Albaek et al., J. Org. Chem., 2006, 71, 7731-7740).

In certain embodiments, sugar surrogates comprise rings having other than 5-atoms. For example, in certain embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA) (see Leumann, CJ. Bioorg. & Med. Chem. (2002) 10:841-854), fluoro HNA (F-HNA), and those compounds having Formula VII:

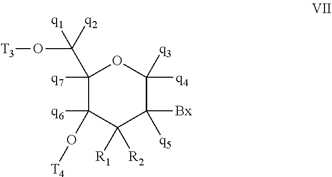

wherein independently for each of said at least one tetrahydropyran nucleoside analog of Formula VII:
Bx is a nucleobase moiety;
T₃ and T₄ are each, independently, an internucleoside linking group linking the tetrahydropyran nucleoside analog to the antisense compound or one of T₃ and T₄ is an internucleoside linking group linking the tetrahydropyran nucleoside analog to the antisense compound and the other of T₃ and T₄ is H, a hydroxyl protecting group, a linked conjugate group, or a 5′ or 3′-terminal group;
   q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is F. In certain embodiments, R₁ is fluoro and R₂ is H, R₁ is methoxy and R₂ is H, and R₁ is methoxyethoxy and R₂ is H.

Many other bicyclo and tricyclo sugar surrogate ring systems are also known in the art that can be used to modify nucleosides for incorporation into antisense compounds (see, e.g., review article: Leumann, J. C, Bioorganic & Medicinal Chemistry, 2002, 10, 841-854).

Combinations of modifications are also provided without limitation, such as 2′-F-5′-methyl substituted nucleosides (see PCT International Application WO 2008/101157 Published on Aug. 21, 2008 for other disclosed 5′, 2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

In certain embodiments, the present invention provides oligonucleotides comprising modified nucleosides. Those modified nucleotides may include modified sugars, modified nucleobases, and/or modified linkages. The specific modifications are selected such that the resulting oligonucleotides possess desirable characteristics. In certain embodiments, oligonucleotides comprise one or more RNA-like nucleosides. In certain embodiments, oligonucleotides comprise one or more DNA-like nucleotides.

ii. Certain Modified Nucleobases

In certain embodiments, nucleosides of the present invention comprise one or more unmodified nucleobases. In certain embodiments, nucleosides of the present invention comprise one or more modified nucleobases.

In certain embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., Angewandte Chemie, International Edition, 1991, 30, 613; and those disclosed by Sanghvi, Y. S., Chapter 15, Antisense Research and Applications, Crooke, S. T. and Lebleu, B., Eds., CRC Press, 1993, 273-288.

Representative United States patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, certain of which are commonly owned with the instant application, and each of which is herein incorporated by reference in its entirety.

b. Certain Internucleoside Linkages

In certain embodiments, nucleosides may be linked together using any internucleoside linkage to form oligonucleotides. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. In certain embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantiomers, diastereomers, and other stereoisomeric configurations that may be defined, in terms of absolute stereochemistry, as (R) or (S), a or p such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

i. 3′-Endo Modifications

In one aspect of the present disclosure, oligomeric compounds include nucleosides synthetically modified to induce a 3′-endo sugar conformation. A nucleoside can incorporate synthetic modifications of the heterocyclic base moiety, the sugar moiety or both to induce a desired 3′-endo sugar conformation. These modified nucleosides are used to mimic RNA like nucleosides so that particular properties of an oligomeric compound can be enhanced while maintaining the desirable 3′-endo conformational geometry. There is an apparent preference for an RNA type duplex (A form helix, predominantly 3′-endo) as a requirement of RNA interference which is supported in part by the fact that duplexes composed of 2′-deoxy-2′-F-nucleosides appear efficient in triggering RNAi response in the C. elegans system. Properties that are enhanced by using more stable 3′-endo nucleosides include but aren't limited to modulation of pharmacokinetic properties through modification of protein binding, protein off-rate, absorption and clearance; modulation of nuclease stability as well as chemical stability; modulation of the binding affinity and specificity of the oligomer (affinity and specificity for enzymes as well as for complementary sequences); and increasing efficacy of RNA cleavage. The present invention provides oligomeric compounds having one or more nucleosides modified in such a way as to favor a C3′-endo type conformation.

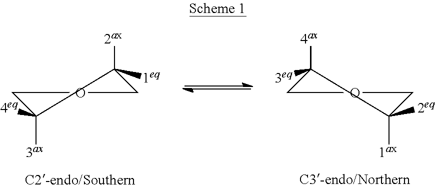

Nucleoside conformation is influenced by various factors including substitution at the 2′, 3′ or 4′-positions of the pentofuranosyl sugar. Electronegative substituents generally prefer the axial positions, while sterically demanding substituents generally prefer the equatorial positions (Principles of Nucleic Acid Structure, Wolfgang Sanger, 1984, Springer-Verlag.) Modification of the 2′ position to favor the 3′-endo conformation can be achieved while maintaining the 2′-OH as a recognition element, as exemplified in Example 35, below (Gallo et al., Tetrahedron (2001), 57, 5707-5713. Harry-O'kuru et al., J. Org. Chem., (1997), 62(6), 1754-1759 and Tang et al., J. Org. Chem. (1999), 64, 747-754.) Alternatively, preference for the 3′-endo conformation can be achieved by deletion of the 2′-OH as exemplified by 2′deoxy-2′F-nucleosides (Kawasaki et al., J. Med. Chem. (1993), 36, 831-841), which adopts the 3′-endo conformation positioning the electronegative fluorine atom in the axial position. Other modifications of the ribose ring, for example substitution at the 4′-position to give 4′-F modified nucleosides (Guillerm et al., Bioorganic and Medicinal Chemistry Letters (1995), 5, 1455-1460 and Owen et al., J. Org. Chem. (1976), 41, 3010-3017), or for example modification to yield methanocarba nucleoside analogs (Jacobson et al., J. Med. Chem. Lett. (2000), 43, 2196-2203 and Lee et al., Bioorganic and Medicinal Chemistry Letters (2001), 11, 1333-1337) also induce preference for the 3′-endo conformation. Some modifications actually lock the conformational geometry by formation of a bicyclic sugar moiety e.g. locked nucleic acid (LNA, Singh et al, Chem. Commun. (1998), 4, 455-456), and ethylene bridged nucleic acids (ENA, Morita et al, Bioorganic & Medicinal Chemistry Letters (2002), 12, 73-76.)

c. Certain Motifs

In certain embodiments, oligomeric compounds comprise or consist of oligonucleotides. In certain embodiments, such oligonucleotides comprise one or more chemical modification. In certain embodiments, chemically modified oligonucleotides comprise one or more modified sugars. In certain embodiments, chemically modified oligonucleotides comprise one or more modified nucleobases. In certain embodiments, chemically modified oligonucleotides comprise one or more modified internucleoside linkages. In certain embodiments, the chemical modifications (sugar modifications, nucleobase modifications, and/or linkage modifications) define a pattern or motif. In certain embodiments, the patterns of chemical modifications of sugar moieties, internucleoside linkages, and nucleobases are each independent of one another. Thus, an oligonucleotide may be described by its sugar modification motif, internucleoside linkage motif and/or nucleobase modification motif (as used herein, nucleobase modification motif describes the chemical modifications to the nucleobases independent of the sequence of nucleobases).

i. Certain Sugar Motifs

In certain embodiments, oligonucleotides comprise one or more type of modified sugar moieties and/or naturally occurring sugar moieties arranged along an oligonucleotide or region thereof in a defined pattern or sugar motif Such sugar motifs include but are not limited to any of the sugar modifications discussed herein.

In certain embodiments, the oligonucleotides comprise or consist of a region having a gapmer sugar motif, which comprises two external regions or “wings” and a central or internal region or “gap.” The three regions of a gapmer sugar motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap. In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric sugar gapmer). In certain embodiments, the sugar motifs of the 5′-wing differs from the sugar motif of the 3′-wing (asymmetric sugar gapmer).

ii. Certain Nucleobase Modification Motifs

In certain embodiments, oligonucleotides comprise chemical modifications to nucleobases arranged along the oligonucleotide or region thereof in a defined pattern or nucleobases modification motif. In certain embodiments, each nucleobase is modified. In certain embodiments, none of the nucleobases is chemically modified.

In certain embodiments, oligonucleotides comprise a block of modified nucleobases. In certain such embodiments, the block is at the 3′-end of the oligonucleotide. In certain embodiments the block is within 3 nucleotides of the 3′-end of the oligonucleotide. In certain such embodiments, the block is at the 5′-end of the oligonucleotide. In certain embodiments the block is within 3 nucleotides of the 5′-end of the oligonucleotide.

In certain embodiments, nucleobase modifications are a function of the natural base at a particular position of an oligonucleotide. For example, in certain embodiments each purine or each pyrimidine in an oligonucleotide is modified. In certain embodiments, each adenine is modified. In certain embodiments, each guanine is modified. In certain embodiments, each thymine is modified. In certain embodiments, each cytosine is modified. In certain embodiments, each uracil is modified.

In certain embodiments, oligonucleotides comprise one or more nucleosides comprising a modified nucleobase. In certain embodiments, oligonucleotides having a gapmer sugar motif comprise a nucleoside comprising a modified nucleobase. In certain such embodiments, one nucleoside comprising a modified nucleobases is in the central gap of an oligonucleotide having a gapmer sugar motif. In certain embodiments, the sugar is an unmodified 2′deoxynucleoside. In certain embodiments, the modified nucleobase is selected from: a 2-thio pyrimidine and a 5-propyne pyrimidine

In certain embodiments, some, all, or none of the cytosine moieties in an oligonucleotide are 5-methyl cytosine moieties. Herein, 5-methyl cytosine is not a “modified nucleobase.” Accordingly, unless otherwise indicated, unmodified nucleobases include both cytosine residues having a 5-methyl and those lacking a 5 methyl. In certain embodiments, the methylation state of all or some cytosine nucleobases is specified.

iii. Certain Nucleoside Motifs

In certain embodiments, oligonucleotides comprise nucleosides comprising modified sugar moieties and/or nucleosides comprising modified nucleobases. Such motifs can be described by their sugar motif and their nucleobase motif separately or by their nucleoside motif, which provides positions or patterns of modified nucleosides (whether modified sugar, nucleobase, or both sugar and nucleobase) in an oligonucleotide.

In certain embodiments, the oligonucleotides comprise or consist of a region having a gapmer nucleoside motif, which comprises two external regions or “wings” and a central or internal region or “gap.” The three regions of a gapmer nucleoside motif (the 5′-wing, the gap, and the 3′-wing) form a contiguous sequence of nucleosides wherein at least some of the sugar moieties and/or nucleobases of the nucleosides of each of the wings differ from at least some of the sugar moieties and/or nucleobase of the nucleosides of the gap. Specifically, at least the nucleosides of each wing that are closest to the gap (the 3′-most nucleoside of the 5′-wing and the 5′-most nucleoside of the 3′-wing) differ from the neighboring gap nucleosides, thus defining the boundary between the wings and the gap. In certain embodiments, the nucleosides within the gap are the same as one another. In certain embodiments, the gap includes one or more nucleoside that differs from one or more other nucleosides of the gap. In certain embodiments, the nucleoside motifs of the two wings are the same as one another (symmetric gapmer). In certain embodiments, the nucleoside motifs of the 5′-wing differs from the nucleoside motif of the 3′-wing (asymmetric gapmer).

iv. Certain 5′-Wings

In certain embodiments, the 5′-wing of a gapmer consists of 1 to 6 linked nucleosides. In certain embodiments, the 5′-wing of a gapmer consists of 1 to 5 linked nucleosides. In certain embodiments, the 5′-wing of a gapmer consists of 2 to 5 linked nucleosides. In certain embodiments, the 5′-wing of a gapmer consists of 3 to 5 linked nucleosides. In certain embodiments, the 5′-wing of a gapmer consists of 4 or 5 linked nucleosides. In certain embodiments, the 5′-wing of a gapmer consists of 1 to 4 linked nucleosides. In certain embodiments, the 5′-wing of a gapmer consists of 1 to 3 linked nucleosides. In certain embodiments, the 5′-wing of a gapmer consists of 1 or 2 linked nucleosides. In certain embodiments, the 5′-wing of a gapmer consists of 2 to 4 linked nucleosides. In certain embodiments, the 5′-wing of a gapmer consists of 2 or 3 linked nucleosides. In certain embodiments, the 5′-wing of a gapmer consists of 3 or 4 linked nucleosides. In certain embodiments, the 5′-wing of a gapmer consists of 1 nucleoside. In certain embodiments, the 5′-wing of a gapmer consists of 2 linked nucleosides. In certain embodiments, the 5′-wing of a gapmer consists of 3 linked nucleosides. In certain embodiments, the 5′-wing of a gapmer consists of 4 linked nucleosides. In certain embodiments, the 5′-wing of a gapmer consists of 5 linked nucleosides. In certain embodiments, the 5′-wing of a gapmer consists of 6 linked nucleosides.

In certain embodiments, the 5′-wing of a gapmer comprises at least one bicyclic nucleoside. In certain embodiments, the 5′-wing of a gapmer comprises at least two bicyclic nucleosides. In certain embodiments, the 5′-wing of a gapmer comprises at least three bicyclic nucleosides. In certain embodiments, the 5′-wing of a gapmer comprises at least four bicyclic nucleosides. In certain embodiments, the 5′-wing of a gapmer comprises at least one constrained ethyl nucleoside. In certain embodiments, the 5′-wing of a gapmer comprises at least one LNA nucleoside. In certain embodiments, each nucleoside of the 5′-wing of a gapmer is a bicyclic nucleoside. In certain embodiments, each nucleoside of the 5′-wing of a gapmer is a constrained ethyl nucleoside. In certain embodiments, each nucleoside of the 5′-wing of a gapmer is a LNA nucleoside.

In certain embodiments, the 5′-wing of a gapmer comprises at least one non-bicyclic modified nucleoside. In certain embodiments, the 5′-wing of a gapmer comprises at least one 2′-substituted nucleoside. In certain embodiments, the 5′-wing of a gapmer comprises at least one 2′-MOE nucleoside. In certain embodiments, the 5′-wing of a gapmer comprises at least one 2′-OMe nucleoside. In certain embodiments, each nucleoside of the 5′-wing of a gapmer is a non-bicyclic modified nucleoside. In certain embodiments, each nucleoside of the 5′-wing of a gapmer is a 2′-substituted nucleoside. In certain embodiments, each nucleoside of the 5′-wing of a gapmer is a 2′-MOE nucleoside. In certain embodiments, each nucleoside of the 5′-wing of a gapmer is a 2′-OMe nucleoside.

In certain embodiments, the 5′-wing of a gapmer comprises at least one 2′-deoxynucleoside. In certain embodiments, each nucleoside of the 5′-wing of a gapmer is a 2′-deoxynucleoside. In a certain embodiments, the 5′-wing of a gapmer comprises at least one ribonucleoside. In certain embodiments, each nucleoside of the 5′-wing of a gapmer is a ribonucleoside. In certain embodiments, one, more than one, or each of the nucleosides of the 5′-wing is an RNA-like nucleoside.

In certain embodiments, the 5′-wing of a gapmer comprises at least one bicyclic nucleoside and at least one non-bicyclic modified nucleoside. In certain embodiments, the 5′-wing of a gapmer comprises at least one bicyclic nucleoside and at least one 2′-substituted nucleoside. In certain embodiments, the 5′-wing of a gapmer comprises at least one bicyclic nucleoside and at least one 2′-MOE nucleoside. In certain embodiments, the 5′-wing of a gapmer comprises at least one bicyclic nucleoside and at least one 2′-OMe nucleoside. In certain embodiments, the 5′-wing of a gapmer comprises at least one bicyclic nucleoside and at least one 2′-deoxynucleoside.

In certain embodiments, the 5′-wing of a gapmer comprises at least one constrained ethyl nucleoside and at least one non-bicyclic modified nucleoside. In certain embodiments, the 5′-wing of a gapmer comprises at least one constrained ethyl nucleoside and at least one 2′-substituted nucleoside. In certain embodiments, the 5′-wing of a gapmer comprises at least one constrained ethyl nucleoside and at least one 2′-MOE nucleoside. In certain embodiments, the 5′-wing of a gapmer comprises at least one constrained ethyl nucleoside and at least one 2′-OMe nucleoside. In certain embodiments, the 5′-wing of a gapmer comprises at least one constrained ethyl nucleoside and at least one 2′-deoxynucleoside.

In certain embodiments, the 5′-wing of a gapmer has a nucleoside motif selected from among the following: ADDA; ABDAA; ABBA; ABB; ABAA; AABAA; AAABAA; AAAABAA; AAAAABAA; AAABAA; AABAA; ABAB; ABADB; ABADDB; AAABB; AAAAA; ABBDC; ABDDC; ABBDCC; ABBDDC; ABBDCC; ABBC; AA; AAA; AAAA; AAAAB; AAAAAAA; AAAAAAAA; ABBB; AB; ABAB; AAAAB; AABBB; AAAAB; and AABBB, wherein each A is a modified nucleoside of a first type, each B is a modified nucleoside of a second type, each C is a modified nucleoside of a third type, and each D is an unmodified deoxynucleoside.

In certain embodiments, the 5′-wing of a gapmer has a nucleoside motif selected from among the following: AB, ABB, AAA, BBB, BBBAA, AAB, BAA, BBAA, AABB, AAAB, ABBW, ABBWW, ABBB, ABBBB, ABAB, ABABAB, ABABBB, ABABAA, AAABB, AAAABB, AABB, AAAAB, AABBB, ABBBB, BBBBB, AAABW, AAAAA, BBBBAA, and AAABW; wherein each A is a modified nucleoside of a first type, each B is a modified nucleoside of a second type, and each W is a modified nucleoside of either the first type, the second type or a third type.

In certain embodiments, the 5′-wing of a gapmer has a nucleoside motif selected from among the following: ABB; ABAA; AABAA; AAABAA; ABAB; ABADB; AAABB; AAAAA; AA; AAA; AAAA; AAAAB; ABBB; AB; and ABAB; wherein each A is a modified nucleoside of a first type, each B is a modified nucleoside of a second type, and each W is a modified nucleoside of either the first type, the second type or a third type.

In certain embodiments, an oligonucleotide comprises any 5′-wing motif provided herein. In certain such embodiments, the oligonucleotide is a 5′-hemimer (does not comprise a 3′-wing). In certain embodiments, such an oligonucleotide is a gapmer. In certain such embodiments, the 3′-wing of the gapmer may comprise any nucleoside motif.

In certain embodiments, the 5′-wing of a gapmer has a sugar motif selected from among those listed in the following non-limiting tables:

TABLE 1
Certain 5′-Wing Sugar Motifs
Certain 5′-Wing Sugar Motifs

| | | | | |
|---|---|---|---|---|
| AAAAA | ABCBB | BABCC | BCBBA | CBACC |
| AAAAB | ABCBC | BACAA | BCBBB | CBBAA |
| AAAAC | ABCCA | BACAB | BCBBC | CBBAB |
| AAABA | ABCCB | BACAC | BCBCA | CBBAC |
| AAABB | ABCCC | BACBA | BCBCB | CBBBA |
| AAABC | ACAAA | BACBB | BCBCC | CBBBB |
| AAACA | ACAAB | BACBC | BCCAA | CBBBC |
| AAACB | ACAAC | BACCA | BCCAB | CBBCA |
| AAACC | ACABA | BACCB | BCCAC | CBBCB |
| AABAA | ACABB | BACCC | BCCBA | CBBCC |
| AABAB | ACABC | BBAAA | BCCBB | CBCAA |
| AABAC | ACACA | BBAAB | BCCBC | CBCAB |
| AABBA | ACACB | BBAAC | BCCCA | CBCAC |
| AABBB | ACACC | BBABA | BCCCB | CBCBA |
| AABBC | ACBAA | BBABB | BCCCC | CBCBB |
| AABCA | ACBAB | BBABC | CAAAA | CBCBC |
| AABCB | ACBAC | BBACA | CAAAB | CBCCA |
| AABCC | ACBBA | BBACB | CAAAC | CBCCB |
| AACAA | ACBBB | BBACC | CAABA | CBCCC |
| AACAB | ACBBC | BBBAA | CAABB | CCAAA |
| AACAC | ACBCA | BBBAB | CAABC | CCAAB |
| AACBA | ACBCB | BBBAC | CAACA | CCAAC |
| AACBB | ACBCC | BBBBA | CAACB | CCABA |
| AACBC | ACCAA | BBBBB | CAACC | CCABB |
| AACCA | ACCAB | BBBBC | CABAA | CCABC |
| AACCB | ACCAC | BBBCA | CABAB | CCACA |
| AACCC | ACCBA | BBBCB | CABAC | CCACB |
| ABAAA | ACCBB | BBBCC | CABBA | CCACC |
| ABAAB | ACCBC | BBCAA | CABBB | CCBAA |
| ABAAC | ACCCA | BBCAB | CABBC | CCBAB |
| ABABA | ACCCB | BBCAC | CABCA | CCBAC |
| ABABB | ACCCC | BBCBA | CABCB | CCBBA |
| ABABC | BAAAA | BBCBB | CABCC | CCBBB |
| ABACA | BAAAB | BBCBC | CACAA | CCBBC |
| ABACB | BAAAC | BBCCA | CACAB | CCBCA |
| ABACC | BAABA | BBCCB | CACAC | CCBCB |
| ABBAA | BAABB | BBCCC | CACBA | CCBCC |
| ABBAB | BAABC | BCAAA | CACBB | CCCAA |
| ABBAC | BAACA | BCAAB | CACBC | CCCAB |
| ABBBA | BAACB | BCAAC | CACCA | CCCAC |
| ABBBB | BAACC | BCABA | CACCB | CCCBA |
| ABBBC | BABAA | BCABB | CACCC | CCCBB |
| ABBCA | BABAB | BCABC | CBAAA | CCCBC |
| ABBCB | BABAC | BCACA | CBAAB | CCCCA |
| ABBCC | BABBA | BCACB | CBAAC | CCCCB |
| ABCAA | BABBB | BCACC | CBABA | CCCCC |
| ABCAB | BABBC | BCBAA | CBABB | |
| ABCAC | BABCA | BCBAB | CBABC | |
| ABCBA | BABCB | BCBAC | CBACA | |

TABLE 2
Certain 5′-Wing Sugar Motifs
Certain 5′-Wing Sugar Motifs

| | | | | |
|---|---|---|---|---|
| AAAAA | BABC | CBAB | ABBB | BAA |
| AAAAB | BACA | CBAC | BAAA | BAB |
| AAABA | BACB | CBBA | BAAB | BBA |
| AAABB | BACC | CBBB | BABA | BBB |
| AABAA | BBAA | CBBC | BABB | AA |
| AABAB | BBAB | CBCA | BBAA | AB |
| AABBA | BBAC | CBCB | BBAB | AC |
| AABBB | BBBA | CBCC | BBBA | BA |
| ABAAA | BBBB | CCAA | BBBB | BB |
| ABAAB | BBBC | CCAB | AAA | BC |
| ABABA | BBCA | CCAC | AAB | CA |
| ABABB | BBCB | CCBA | AAC | CB |
| ABBAA | BBCC | CCBB | ABA | CC |
| ABBAB | BCAA | CCBC | ABB | AA |
| ABBBA | BCAB | CCCA | ABC | AB |
| ABBBB | BCAC | CCCB | ACA | BA |
| BAAAA | ABCB | BCBA | ACB | |
| BAAAB | ABCC | BCBB | ACC | |
| BAABA | ACAA | BCBC | BAA | |
| BAABB | ACAB | BCCA | BAB | |
| BABAA | ACAC | BCCB | BAC | |
| BABAB | ACBA | BCCC | BBA | |
| BABBA | ACBB | CAAA | BBB | |
| BABBB | ACBC | CAAB | BBC | |
| BBAAA | ACCA | CAAC | BCA | |
| BBAAB | ACCB | CABA | BCB | |
| BBABA | ACCC | CABB | BCC | |
| BBABB | BAAA | CABC | CAA | |
| BBBAA | BAAB | CACA | CAB | |
| BBBAB | BAAC | CACB | CAC | |
| BBBBA | BABA | CACC | CBA | |
| BBBBB | BABB | CBAA | CBB | |
| AAAA | AACC | CCCC | CBC | |
| AAAB | ABAA | AAAA | CCA | |
| AAAC | ABAB | AAAB | CCB | |
| AABA | ABAC | AABA | CCC | |
| AABB | ABBA | AABB | AAA | |
| AABC | ABBB | ABAA | AAB | |
| AACA | ABBC | ABAB | ABA | |
| AACB | ABCA | ABBA | ABB | |

In certain embodiments, each A, each B, and each C located at the 3′-most 5′-wing nucleoside is a modified nucleoside. For example, in certain embodiments the 5′-wing motif is selected from among ABB, BBB, and CBB, wherein the underlined nucleoside represents the 3′-most 5′-wing nucleoside and wherein the underlined nucleoside is a modified nucleoside. In certain embodiments, the 3′-most 5′-wing nucleoside comprises a bicyclic sugar moiety selected from among cEt, cMOE, LNA, α-L-LNA, ENA and 2′-thio LNA. In certain embodiments, the 3′-most 5′-wing nucleoside comprises a bicyclic sugar moiety selected from among cEt and LNA. In certain embodiments, the 3′-most 5′-wing nucleoside comprises cEt. In certain embodiments, the 3′-most 5′-wing nucleoside comprises LNA.

In certain embodiments, each A comprises an unmodified 2′-deoxyfuranose sugar moiety. In certain embodiments, each A comprises a modified sugar moiety. In certain embodiments, each A comprises a 2′-substituted sugar moiety. In certain embodiments, each A comprises a 2′-substituted sugar moiety selected from among F, ara-F, OCH₃ and O(CH₂)₂—OCH₃. In certain embodiments, each A comprises a bicyclic sugar moiety. In certain embodiments, each A comprises a bicyclic sugar moiety selected from among cEt, cMOE, LNA, α-L-LNA, ENA and 2′-thio LNA. In certain embodiments, each A comprises a modified nucleobase. In certain embodiments, each A comprises a modified nucleobase selected from among 2-thio-thymidine nucleoside and 5-propyne uridine nucleoside. In certain embodiments, each A comprises an HNA. In certain embodiments, each A comprises a F-HNA. In certain embodiments, each A comprises a 5′-substituted sugar moiety selected from among 5′-Me DNA, and 5′-(R)-Me DNA.

In certain embodiments, each B comprises an unmodified 2′-deoxyfuranose sugar moiety. In certain embodiments, each B comprises a modified sugar moiety. In certain embodiments, each B comprises a 2′-substituted sugar moiety. In certain embodiments, each B comprises a 2′-substituted sugar moiety selected from among F, (ara)-F, OCH₃ and O(CH₂)₂—OCH₃. In certain embodiments, each B comprises a bicyclic sugar moiety. In certain embodiments, each B comprises a bicyclic sugar moiety selected from among cEt, cMOE, LNA, α-L-LNA, ENA and 2′-thio LNA. In certain embodiments, each B comprises a modified nucleobase. In certain embodiments, each B comprises a modified nucleobase selected from among 2-thio-thymidine nucleoside and 5-propyne uridine nucleoside. In certain embodiments, each B comprises an HNA. In certain embodiments, each B comprises a F-HNA. In certain embodiments, each B comprises a 5′-substituted sugar moiety selected from among 5′-Me DNA, and 5′-(R)-Me DNA.

In certain embodiments, each A comprises a 2′-substituted sugar moiety selected from among F, ara-F, OCH₃ and O(CH₂)₂—OCH₃ and each B comprises a bicyclic sugar moiety selected from among cEt, cMOE, LNA, α-L-LNA, ENA and 2′-thio LNA. In certain embodiments, each A comprises O(CH₂)₂—OCH₃ and each B comprises cEt.

In certain embodiments, each C comprises an unmodified 2′-deoxyfuranose sugar moiety. In certain embodiments, each C comprises a modified sugar moiety. In certain embodiments, each C comprises a 2′-substituted sugar moiety. In certain embodiments, each C comprises a 2′-substituted sugar moiety selected from among F, (ara)-F, OCH₃ and O(CH₂)₂—OCH₃. In certain embodiments, each C comprises a 5′-substituted sugar moiety. In certain embodiments, each C comprises a 5′-substituted sugar moiety selected from among 5′-Me DNA, and 5′-(R)-Me DNA. In certain embodiments, each C comprises a bicyclic sugar moiety. In certain embodiments, each C comprises a bicyclic sugar moiety selected from among cEt, cMOE, LNA, α-L-LNA, ENA and 2′-thio LNA. In certain embodiments, each C comprises a modified nucleobase. In certain embodiments, each C comprises a modified nucleobase selected from among 2-thio-thymidine and 5-propyne uridine. In certain embodiments, each C comprises a 2-thio-thymidine nucleoside. In certain embodiments, each C comprises an HNA. In certain embodiments, each C comprises an F-HNA.

v. Certain 3′-Wings

In certain embodiments, the 3′-wing of a gapmer consists of 1 to 6 linked nucleosides. In certain embodiments, the 3′-wing of a gapmer consists of 1 to 5 linked nucleosides. In certain embodiments, the 3′-wing of a gapmer consists of 2 to 5 linked nucleosides. In certain embodiments, the 3′-wing of a gapmer consists of 3 to 5 linked nucleosides. In certain embodiments, the 3′-wing of a gapmer consists of 4 or 5 linked nucleosides. In certain embodiments, the 3′-wing of a gapmer consists of 1 to 4 linked nucleosides. In certain embodiments, the 3′-wing of a gapmer consists of 1 to 3 linked nucleosides. In certain embodiments, the 3′-wing of a gapmer consists of 1 or 2 linked nucleosides. In certain embodiments, the 3′-wing of a gapmer consists of 2 to 4 linked nucleosides. In certain embodiments, the 3′-wing of a gapmer consists of 2 or 3 linked nucleosides. In certain embodiments, the 3′-wing of a gapmer consists of 3 or 4 linked nucleosides. In certain embodiments, the 3′-wing of a gapmer consists of 1 nucleoside. In certain embodiments, the 3′-wing of a gapmer consists of 2 linked nucleosides. In certain embodiments, the 3′-wing of a gapmer consists of 3 linked nucleosides. In certain embodiments, the 3′-wing of a gapmer consists of 4 linked nucleosides. In certain embodiments, the 3′-wing of a gapmer consists of 5 linked nucleosides. In certain embodiments, the 3′-wing of a gapmer consists of 6 linked nucleosides.

In certain embodiments, the 3′-wing of a gapmer comprises at least one bicyclic nucleoside. In certain embodiments, the 3′-wing of a gapmer comprises at least one constrained ethyl nucleoside. In certain embodiments, the 3′-wing of a gapmer comprises at least one LNA nucleoside. In certain embodiments, each nucleoside of the 3′-wing of a gapmer is a bicyclic nucleoside. In certain embodiments, each nucleoside of the 3′-wing of a gapmer is a constrained ethyl nucleoside. In certain embodiments, each nucleoside of the 3′-wing of a gapmer is a LNA nucleoside.

In certain embodiments, the 3′-wing of a gapmer comprises at least one non-bicyclic modified nucleoside. In certain embodiments, the 3′-wing of a gapmer comprises at least two non-bicyclic modified nucleosides. In certain embodiments, the 3′-wing of a gapmer comprises at least three non-bicyclic modified nucleosides. In certain embodiments, the 3′-wing of a gapmer comprises at least four non-bicyclic modified nucleosides. In certain embodiments, the 3′-wing of a gapmer comprises at least one 2′-substituted nucleoside. In certain embodiments, the 3′-wing of a gapmer comprises at least one 2′-MOE nucleoside. In certain embodiments, the 3′-wing of a gapmer comprises at least one 2′-OMe nucleoside. In certain embodiments, each nucleoside of the 3′-wing of a gapmer is a non-bicyclic modified nucleoside. In certain embodiments, each nucleoside of the 3′-wing of a gapmer is a 2′-substituted nucleoside. In certain embodiments, each nucleoside of the 3′-wing of a gapmer is a 2′-MOE nucleoside. In certain embodiments, each nucleoside of the 3′-wing of a gapmer is a 2′-OMe nucleoside.

In certain embodiments, the 3′-wing of a gapmer comprises at least one 2′-deoxynucleoside. In certain embodiments, each nucleoside of the 3′-wing of a gapmer is a 2′-deoxynucleoside. In a certain embodiments, the 3′-wing of a gapmer comprises at least one ribonucleoside. In certain embodiments, each nucleoside of the 3′-wing of a gapmer is a ribonucleoside. In certain embodiments, one, more than one, or each of the nucleosides of the 5′-wing is an RNA-like nucleoside.

In certain embodiments, the 3′-wing of a gapmer comprises at least one bicyclic nucleoside and at least one non-bicyclic modified nucleoside. In certain embodiments, the 3′-wing of a gapmer comprises at least one bicyclic nucleoside and at least one 2′-substituted nucleoside. In certain embodiments, the 3′-wing of a gapmer comprises at least one bicyclic nucleoside and at least one 2′-MOE nucleoside. In certain embodiments, the 3′-wing of a gapmer comprises at least one bicyclic nucleoside and at least one 2′-OMe nucleoside. In certain embodiments, the 3′-wing of a gapmer comprises at least one bicyclic nucleoside and at least one 2′-deoxynucleoside.

In certain embodiments, the 3′-wing of a gapmer comprises at least one constrained ethyl nucleoside and at least one non-bicyclic modified nucleoside. In certain embodiments, the 3′-wing of a gapmer comprises at least one constrained ethyl nucleoside and at least one 2′-substituted nucleoside. In certain embodiments, the 3′-wing of a gapmer comprises at least one constrained ethyl nucleoside and at least one 2′-MOE nucleoside. In certain embodiments, the 3′-wing of a gapmer comprises at least one constrained ethyl nucleoside and at least one 2′-OMe nucleoside. In certain embodiments, the 3′-wing of a gapmer comprises at least one constrained ethyl nucleoside and at least one 2′-deoxynucleoside.

In certain embodiments, the 3′-wing of a gapmer comprises at least one LNA nucleoside and at least one non-bicyclic modified nucleoside. In certain embodiments, the 3′-wing of a gapmer comprises at least one LNA nucleoside and at least one 2′-substituted nucleoside. In certain embodiments, the 3′-wing of a gapmer comprises at least one LNA nucleoside and at least one 2′-MOE nucleoside. In certain embodiments, the 3′-wing of a gapmer comprises at least one LNA nucleoside and at least one 2′-OMe nucleoside. In certain embodiments, the 3′-wing of a gapmer comprises at least one LNA nucleoside and at least one 2′-deoxynucleoside.

In certain embodiments, the 3′-wing of a gapmer comprises at least one bicyclic nucleoside, at least one non-bicyclic modified nucleoside, and at least one 2′-deoxynucleoside. In certain embodiments, the 3′-wing of a gapmer comprises at least one constrained ethyl nucleoside, at least one non-bicyclic modified nucleoside, and at least one 2′-deoxynucleoside. In certain embodiments, the 3′-wing of a gapmer comprises at least one LNA nucleoside, at least one non-bicyclic modified nucleoside, and at least one 2′-deoxynucleoside.

In certain embodiments, the 3′-wing of a gapmer comprises at least one bicyclic nucleoside, at least one 2′-substituted nucleoside, and at least one 2′-deoxynucleoside. In certain embodiments, the 3′-wing of a gapmer comprises at least one constrained ethyl nucleoside, at least one 2′-substituted nucleoside, and at least one 2′-deoxynucleoside. In certain embodiments, the 3′-wing of a gapmer comprises at least one LNA nucleoside, at least one 2′-substituted nucleoside, and at least one 2′-deoxynucleoside.

In certain embodiments, the 3′-wing of a gapmer comprises at least one bicyclic nucleoside, at least one 2′-MOE nucleoside, and at least one 2′-deoxynucleoside. In certain embodiments, the 3′-wing of a gapmer comprises at least one constrained ethyl nucleoside, at least one 2′-MOE nucleoside, and at least one 2′-deoxynucleoside. In certain embodiments, the 3′-wing of a gapmer comprises at least one LNA nucleoside, at least one 2′-MOE nucleoside, and at least one 2′-deoxynucleoside.

In certain embodiments, the 3′-wing of a gapmer comprises at least one bicyclic nucleoside, at least one 2′-OMe nucleoside, and at least one 2′-deoxynucleoside. In certain embodiments, the 3′-wing of a gapmer comprises at least one constrained ethyl nucleoside, at least one 2′-OMe nucleoside, and at least one 2′-deoxynucleoside. In certain embodiments, the 3′-wing of a gapmer comprises at least one LNA nucleoside, at least one 2′-OMe nucleoside, and at least one 2′-deoxynucleoside.

In certain embodiments, the 3′-wing of a gapmer has a nucleoside motif selected from among the following: ABB, ABAA, AAABAA, AAAAABAA, AABAA, AAAABAA, AAABAA, ABAB, AAAAA, AAABB, AAAAAAAA, AAAAAAA, AAAAAA, AAAAB, AAAA, AAA, AA, AB, ABBB, ABAB, AABBB; wherein each A is a modified nucleoside of a first type, each B is a modified nucleoside of a second type. In certain embodiments, an oligonucleotide comprises any 3′-wing motif provided herein. In certain such embodiments, the oligonucleotide is a 3′-hemimer (does not comprise a 5′-wing). In certain embodiments, such an oligonucleotide is a gapmer. In certain such embodiments, the 5′-wing of the gapmer may comprise any nucleoside motif.

In certain embodiments, the 3′-wing of a gapmer has a nucleoside motif selected from among the following: BBA, AAB, AAA, BBB, BBAA, AABB, WBBA, WAAB, BBBA, BBBBA, BBBB, BBBBBA, ABBBBB, BBAAA, AABBB, BBBAA, BBBBA, BBBBB, BABA, AAAAA, BBAAAA, AABBBB, BAAAA, and ABBBB, wherein each A is a modified nucleoside of a first type, each B is a modified nucleoside of a second type, and each W is a modified nucleoside of either the first type, the second type or a third type.

In certain embodiments, the 3′-wing of a gapmer has a nucleoside motif selected from among the following: ABB; AAABAA; AABAA; AAAABAA; AAAAA; AAABB; AAAAAAAA; AAAAAAA; AAAAAA; AAAAB; AB; ABBB; and ABAB, wherein each A is a modified nucleoside of a first type, each B is a modified nucleoside of a second type, and each W is a modified nucleoside of either the first type, the second type or a third type.

In certain embodiments, the 3′-wing of a gapmer has a sugar motif selected from among those listed in the following non-limiting tables:

TABLE 3
Certain 3′-Wing Sugar Motifs
Certain 3′-Wing Sugar Motifs

| | | | | |
|---|---|---|---|---|
| AAAAA | ABCBB | BABCC | BCBBA | CBACC |
| AAAAB | ABCBC | BACAA | BCBBB | CBBAA |
| AAAAC | ABCCA | BACAB | BCBBC | CBBAB |
| AAABA | ABCCB | BACAC | BCBCA | CBBAC |
| AAABB | ABCCC | BACBA | BCBCB | CBBBA |
| AAABC | ACAAA | BACBB | BCBCC | CBBBB |
| AAACA | ACAAB | BACBC | BCCAA | CBBBC |
| AAACB | ACAAC | BACCA | BCCAB | CBBCA |
| AAACC | ACABA | BACCB | BCCAC | CBBCB |
| AABAA | ACABB | BACCC | BCCBA | CBBCC |
| AABAB | ACABC | BBAAA | BCCBB | CBCAA |
| AABAC | ACACA | BBAAB | BCCBC | CBCAB |
| AABBA | ACACB | BBAAC | BCCCA | CBCAC |
| AABBB | ACACC | BBABA | BCCCB | CBCBA |
| AABBC | ACBAA | BBABB | BCCCC | CBCBB |
| AABCA | ACBAB | BBABC | CAAAA | CBCBC |
| AABCB | ACBAC | BBACA | CAAAB | CBCCA |
| AABCC | ACBBA | BBACB | CAAAC | CBCCB |
| AACAA | ACBBB | BBACC | CAABA | CBCCC |
| AACAB | ACBBC | BBBAA | CAABB | CCAAA |
| AACAC | ACBCA | BBBAB | CAABC | CCAAB |
| AACBA | ACBCB | BBBAC | CAACA | CCAAC |
| AACBB | ACBCC | BBBBA | CAACB | CCABA |
| AACBC | ACCAA | BBBBB | CAACC | CCABB |
| AACCA | ACCAB | BBBBC | CABAA | CCABC |
| AACCB | ACCAC | BBBCA | CABAB | CCACA |
| AACCC | ACCBA | BBBCB | CABAC | CCACB |
| ABAAA | ACCBB | BBBCC | CABBA | CCACC |
| ABAAB | ACCBC | BBCAA | CABBB | CCBAA |
| ABAAC | ACCCA | BBCAB | CABBC | CCBAB |
| ABABA | ACCCB | BBCAC | CABCA | CCBAC |
| ABABB | ACCCC | BBCBA | CABCB | CCBBA |
| ABABC | BAAAA | BBCBB | CABCC | CCBBB |
| ABACA | BAAAB | BBCBC | CACAA | CCBBC |
| ABACB | BAAAC | BBCCA | CACAB | CCBCA |
| ABACC | BAABA | BBCCB | CACAC | CCBCB |
| ABBAA | BAABB | BBCCC | CACBA | CCBCC |
| ABBAB | BAABC | BCAAA | CACBB | CCCAA |
| ABBAC | BAACA | BCAAB | CACBC | CCCAB |
| ABBBA | BAACB | BCAAC | CACCA | CCCAC |
| ABBBB | BAACC | BCABA | CACCB | CCCBA |
| ABBBC | BABAA | BCABB | CACCC | CCCBB |
| ABBCA | BABAB | BCABC | CBAAA | CCCBC |
| ABBCB | BABAC | BCACA | CBAAB | CCCCA |
| ABBCC | BABBA | BCACB | CBAAC | CCCCB |
| ABCAA | BABBB | BCACC | CBABA | CCCCC |
| ABCAB | BABBC | BCBAA | CBABB | |
| ABCAC | BABCA | BCBAB | CBABC | |
| ABCBA | BABCB | BCBAC | CBACA | |

TABLE 4
Certain 3′-Wing Sugar Motifs
Certain 3′-Wing Sugar Motifs

| | | | | |
|---|---|---|---|---|
| AAAAA | BABC | CBAB | ABBB | BAA |
| AAAAB | BACA | CBAC | BAAA | BAB |
| AAABA | BACB | CBBA | BAAB | BBA |
| AAABB | BACC | CBBB | BABA | BBB |
| AABAA | BBAA | CBBC | BABB | AA |
| AABAB | BBAB | CBCA | BBAA | AB |
| AABBA | BBAC | CBCB | BBAB | AC |
| AABBB | BBBA | CBCC | BBBA | BA |
| ABAAA | BBBB | CCAA | BBBB | BB |
| ABAAB | BBBC | CCAB | AAA | BC |
| ABABA | BBCA | CCAC | AAB | CA |
| ABABB | BBCB | CCBA | AAC | CB |
| ABBAA | BBCC | CCBB | ABA | CC |
| ABBAB | BCAA | CCBC | ABB | AA |
| ABBBA | BCAB | CCCA | ABC | AB |
| ABBBB | BCAC | CCCB | ACA | BA |
| BAAAA | ABCB | BCBA | ACB | |
| BAAAB | ABCC | BCBB | ACC | |
| BAABA | ACAA | BCBC | BAA | |
| BAABB | ACAB | BCCA | BAB | |
| BABAA | ACAC | BCCB | BAC | |
| BABAB | ACBA | BCCC | BBA | |
| BABBA | ACBB | CAAA | BBB | |
| BABBB | ACBC | CAAB | BBC | |
| BBAAA | ACCA | CAAC | BCA | |
| BBAAB | ACCB | CABA | BCB | |
| BBABA | ACCC | CABB | BCC | |
| BBABB | BAAA | CABC | CAA | |
| BBBAA | BAAB | CACA | CAB | |
| BBBAB | BAAC | CACB | CAC | |
| BBBBA | BABA | CACC | CBA | |
| BBBBB | BABB | CBAA | CBB | |
| AAAA | AACC | CCCC | CBC | |
| AAAB | ABAA | AAAA | CCA | |
| AAAC | ABAB | AAAB | CCB | |
| AABA | ABAC | AABA | CCC | |
| AABB | ABBA | AABB | AAA | |
| AABC | ABBB | ABAA | AAB | |
| AACA | ABBC | ABAB | ABA | |
| AACB | ABCA | ABBA | ABB | |

In certain embodiments, each A, each B, and each C located at the 5′-most 3′-wing region nucleoside is a modified nucleoside. For example, in certain embodiments the 3′-wing motif is selected from among ABB, BBB, and CBB, wherein the underlined nucleoside represents the 5′-most 3′-wing region nucleoside and wherein the underlined nucleoside is a modified nucleoside.

In certain embodiments, each A comprises an unmodified 2′-deoxyfuranose sugar moiety. In certain embodiments, each A comprises a modified sugar moiety. In certain embodiments, each A comprises a 2′-substituted sugar moiety. In certain embodiments, each A comprises a 2′-substituted sugar moiety selected from among F, ara-F, OCH₃ and O(CH₂)₂—OCH₃. In certain embodiments, each A comprises a bicyclic sugar moiety. In certain embodiments, each A comprises a bicyclic sugar moiety selected from among cEt, cMOE, LNA, α-L-LNA, ENA and 2′-thio LNA. In certain embodiments, each A comprises a modified nucleobase. In certain embodiments, each A comprises a modified nucleobase selected from among 2-thio-thymidine nucleoside and 5-propyne uridine nucleoside. In certain embodiments, each A comprises a 5′-substituted sugar moiety selected from among 5′-Me DNA, and 5′-(R)-Me DNA.

In certain embodiments, each B comprises an unmodified 2′-deoxyfuranose sugar moiety. In certain embodiments, each B comprises a modified sugar moiety. In certain embodiments, each B comprises a 2′-substituted sugar moiety. In certain embodiments, each B comprises a 2′-substituted sugar moiety selected from among F, (ara)-F, OCH₃ and O(CH₂)₂—OCH₃. In certain embodiments, each B comprises a bicyclic sugar moiety. In certain embodiments, each B comprises a bicyclic sugar moiety selected from among cEt, cMOE, LNA, α-L-LNA, ENA and 2′-thio LNA. In certain embodiments, each B comprises a modified nucleobase. In certain embodiments, each B comprises a modified nucleobase selected from among 2-thio-thymidine nucleoside and 5-propyne uridine nucleoside. In certain embodiments, each B comprises an HNA. In certain embodiments, each B comprises an F-HNA. In certain embodiments, each B comprises a 5′-substituted sugar moiety selected from among 5′-Me DNA, and 5′-(R)-Me DNA.

In certain embodiments, each A comprises a 2′-substituted sugar moiety selected from among F, ara-F, OCH₃ and O(CH₂)₂—OCH₃ and each B comprises a bicyclic sugar moiety selected from among cEt, cMOE, LNA, α-L-LNA, ENA and 2′-thio LNA. In certain embodiments, each A comprises O(CH₂)₂—OCH₃ and each B comprises cEt.

In certain embodiments, each C comprises an unmodified 2′-deoxyfuranose sugar moiety. In certain embodiments, each C comprises a modified sugar moiety. In certain embodiments, each C comprises a 2′-substituted sugar moiety. In certain embodiments, each C comprises a 2′-substituted sugar moiety selected from among F, (ara)-F, OCH₃ and O(CH₂)₂—OCH₃. In certain embodiments, each C comprises a 5′-substituted sugar moiety. In certain embodiments, each C comprises a 5′-substituted sugar moiety selected from among 5′-Me, and 5′-(R)-Me. In certain embodiments, each C comprises a bicyclic sugar moiety. In certain embodiments, each C comprises a bicyclic sugar moiety selected from among cEt, cMOE, LNA, α-L-LNA, ENA and 2′-thio LNA. In certain embodiments, each C comprises a modified nucleobase. In certain embodiments, each C comprises a modified nucleobase selected from among 2-thio-thymidine and 5-propyne uridine. In certain embodiments, each C comprises a 2-thio-thymidine nucleoside. In certain embodiments, each C comprises an HNA. In certain embodiments, each C comprises an F-HNA.

vi. Certain Central Regions (Gaps)

In certain embodiments, the gap of a gapmer consists of 6 to 20 linked nucleosides. In certain embodiments, the gap of a gapmer consists of 6 to 15 linked nucleosides. In certain embodiments, the gap of a gapmer consists of 6 to 12 linked nucleosides. In certain embodiments, the gap of a gapmer consists of 6 to 10 linked nucleosides. In certain embodiments, the gap of a gapmer consists of 6 to 9 linked nucleosides. In certain embodiments, the gap of a gapmer consists of 6 to 8 linked nucleosides. In certain embodiments, the gap of a gapmer consists of 6 or 7 linked nucleosides. In certain embodiments, the gap of a gapmer consists of 7 to 10 linked nucleosides. In certain embodiments, the gap of a gapmer consists of 7 to 9 linked nucleosides. In certain embodiments, the gap of a gapmer consists of 7 or 8 linked nucleosides. In certain embodiments, the gap of a gapmer consists of 8 to 10 linked nucleosides. In certain embodiments, the gap of a gapmer consists of 8 or 9 linked nucleosides. In certain embodiments, the gap of a gapmer consists of 6 linked nucleosides. In certain embodiments, the gap of a gapmer consists of 7 linked nucleosides. In certain embodiments, the gap of a gapmer consists of 8 linked nucleosides. In certain embodiments, the gap of a gapmer consists of 9 linked nucleosides. In certain embodiments, the gap of a gapmer consists of 10 linked nucleosides. In certain embodiments, the gap of a gapmer consists of 11 linked nucleosides. In certain embodiments, the gap of a gapmer consists of 12 linked nucleosides.

In certain embodiments, each nucleoside of the gap of a gapmer is a 2′-deoxynucleoside. In certain embodiments, the gap comprises one or more modified nucleosides. In certain embodiments, each nucleoside of the gap of a gapmer is a 2′-deoxynucleoside or is a modified nucleoside that is “DNA-like.” In such embodiments, “DNA-like” means that the nucleoside has similar characteristics to DNA, such that a duplex comprising the gapmer and an RNA molecule is capable of activating RNase H. For example, under certain conditions, 2′-(ara)-F have been shown to support RNase H activation, and thus is DNA-like. In certain embodiments, one or more nucleosides of the gap of a gapmer is not a 2′-deoxynucleoside and is not DNA-like. In certain such embodiments, the gapmer nonetheless supports RNase H activation (e.g., by virtue of the number or placement of the non-DNA nucleosides).

In certain embodiments, gaps comprise a stretch of unmodified 2′-deoxynucleoside interrupted by one or more modified nucleosides, thus resulting in three sub-regions (two stretches of one or more 2′-deoxynucleosides and a stretch of one or more interrupting modified nucleosides). In certain embodiments, no stretch of unmodified 2′-deoxynucleosides is longer than 5, 6, or 7 nucleosides. In certain embodiments, such short stretches is achieved by using short gap regions. In certain embodiments, short stretches are achieved by interrupting a longer gap region.

In certain embodiments, the gap comprises one or more modified nucleosides. In certain embodiments, the gap comprises one or more modified nucleosides selected from among cEt, FHNA, LNA, and 2-thio-thymidine. In certain embodiments, the gap comprises one modified nucleoside. In certain embodiments, the gap comprises a 5′-substituted sugar moiety selected from among 5′-Me, and 5′-(R)-Me. In certain embodiments, the gap comprises two modified nucleosides. In certain embodiments, the gap comprises three modified nucleosides. In certain embodiments, the gap comprises four modified nucleosides. In certain embodiments, the gap comprises two or more modified nucleosides and each modified nucleoside is the same. In certain embodiments, the gap comprises two or more modified nucleosides and each modified nucleoside is different.

In certain embodiments, the gap comprises one or more modified linkages. In certain embodiments, the gap comprises one or more methyl phosphonate linkages. In certain embodiments the gap comprises two or more modified linkages. In certain embodiments, the gap comprises one or more modified linkages and one or more modified nucleosides. In certain embodiments, the gap comprises one modified linkage and one modified nucleoside. In certain embodiments, the gap comprises two modified linkages and two or more modified nucleosides.

In certain embodiments, the gap comprises a nucleoside motif selected from among the following: DDDDXDDDDD; DDDDDXDDDDD; DDDXDDDDD; DDDDXDDDDDD; DDDDXDDDD; DDXDDDDDD; DDDXDDDDDD; DXDDDDDD; DDXDDDDDDD; DDXDDDDD; DDXDDDXDDD; DDDXDDDXDDD; DXDDDXDDD; DDXDDDXDD; DDXDDDDXDDD; DDXDDDDXDD; DXDDDDXDDD; DDDDXDDD; DDDXDDD; DXDDDDDDD; DDDDXXDDD; and DXXDXXDXX; wherein each D is an unmodified deoxynucleoside; and each X is a modified nucleoside or a substituted sugar moiety.

In certain embodiments, the gap comprises a nucleoside motif selected from among the following: DDDDDDDDD; DXDDDDDDD; DDXDDDDDD; DDDXDDDDD; DDDDXDDDD; DDDDDXDDD; DDDDDDXDD; DDDDDDDXD; DXXDDDDDD; DDDDDDXXD; DDXXDDDDD; DDDXXDDDD; DDDDXXDDD; DDDDDXXDD; DXDDDDDXD; DXDDDDXDD; DXDDDXDDD; DXDDXDDDD; DXDXDDDDD; DDXDDDDXD; DDXDDDXDD; DDXDDXDDD; DDXDXDDDD; DDDXDDDXD; DDDXDDXDD; DDDXDXDDD; DDDDXDDXD; DDDDXDXDD; and DDDDDXDXD, wherein each D is an unmodified deoxynucleoside; and each X is a modified nucleoside or a substituted sugar moiety.

In certain embodiments, the gap comprises a nucleoside motif selected from among the following: DDDDXDDDD, DXDDDDDDD, DXXDDDDDD, DDXDDDDDD, DDDXDDDDD, DDDDXDDDD, DDDDDXDDD, DDDDDDXDD, and DDDDDDDXD, wherein each D is an unmodified deoxynucleoside; and each X is a modified nucleoside or a substituted sugar moiety.

In certain embodiments, the gap comprises a nucleoside motif selected from among the following: DDDDDDDD, DXDDDDDD, DDXDDDDD, DDDXDDDD, DDDDXDDD, DDDDDXDD, DDDDDDXD, DXDDDDXD, DXDDDXDD, DXDDXDDD, DXDXDDDD, DXXDDDDD, DDXXDDDD, DDXDXDDD, DDXDDXDD, DXDDDDXD, DDDXXDDD, DDDXDXDD, DDDXDDXD, DDDDXXDD, DDDDXDXD, and DDDDDXXD, wherein each D is an unmodified deoxynucleoside; and each X is a modified nucleoside or a substituted sugar moiety.

In certain embodiments, the gap comprises a nucleoside motif selected from among the following: DXDDDDD, DDXDDDD, DDDXDDD, DDDDXDD, DDDDDXD, DXDDDXD, DXDDXDD, DXDXDDD, DXXDDDD, DDXXDDD, DDXDXDD, DDXDDXD, DDDXXDD, DDDXDXD, and DDDDXXD, wherein each D is an unmodified deoxynucleoside; and each X is a modified nucleoside or a substituted sugar moiety.

In certain embodiments, the gap comprises a nucleoside motif selected from among the following: DXDDDD, DDXDDD, DDDXDD, DDDDXD, DXXDDD, DXDXDD, DXDDXD, DDXXDD, DDXDXD, and DDDXXD, wherein each D is an unmodified deoxynucleoside; and each X is a modified nucleoside or a substituted sugar moiety.

In certain embodiments, the gap comprises a nucleoside motif selected from among the following: DXDDDD, DDXDDD, DDDXDD, DDDDXD, DXDDDDD, DDXDDDD, DDDXDDD, DDDDXDD, DDDDDXD, DXDDDDDD, DDXDDDDD, DDDXDDDD, DDDDXDDD, DDDDDXDD, DDDDDDXD, DXDDDDDDD; DDXDDDDDD, DDDXDDDDD, DDDDXDDDD, DDDDDXDDD, DDDDDDXDD, DDDDDDDXD, DXDDDDDDDD, DDXDDDDDDD, DDDXDDDDDD, DDDDXDDDDD, DDDDDXDDDD, DDDDDDXDDD, DDDDDDDXDD, and DDDDDDDDXD, wherein each D is an unmodified deoxynucleoside; and each X is a modified nucleoside or a substituted sugar moiety.

In certain embodiments, each X comprises an unmodified 2′-deoxyfuranose sugar moiety. In certain embodiments, each X comprises a modified sugar moiety. In certain embodiments, each X comprises a 2′-substituted sugar moiety. In certain embodiments, each X comprises a 2′-substituted sugar moiety selected from among F, (ara)-F, OCH₃ and O(CH₂)₂—OCH₃. In certain embodiments, each X comprises a 5′-substituted sugar moiety. In certain embodiments, each X comprises a 5′-substituted sugar moiety selected from among 5′-Me, and 5′-(R)-Me. In certain embodiments, each X comprises a bicyclic sugar moiety. In certain embodiments, each X comprises a bicyclic sugar moiety selected from among cEt, cMOE, LNA, α-L-LNA, ENA and 2′-thio LNA. In certain embodiments, each X comprises a modified nucleobase. In certain embodiments, each X comprises a modified nucleobase selected from among 2-thio-thymidine and 5-propyne uridine. In certain embodiments, each X comprises a 2-thio-thymidine nucleoside. In certain embodiments, each X comprises an HNA. In certain embodiments, each C comprises an F-HNA. In certain embodiments, X represents the location of a single differentiating nucleobase.

vii. Certain Gapmer Motifs

In certain embodiments, a gapmer comprises a 5′-wing, a gap, and a 3′ wing, wherein the 5′-wing, gap, and 3′ wing are independently selected from among those discussed above. For example, in certain embodiments, a gapmer has a 5′-wing, a gap, and a 3′-wing having features selected from among any of those listed in the tables above and any 5′-wing may be paired with any gap and any 3′-wing. For example, in certain embodiments, a 5′-wing may comprise AAABB, a 3′-wing may comprise BBA, and the gap may comprise DDDDDDD. For example, in certain embodiments, a gapmer has a 5′-wing, a gap, and a 3′-wing having features selected from among those listed in the following non-limiting table, wherein each motif is represented as (5′-wing)-(gap)-(3′-wing), wherein each number represents the number of linked nucleosides in each portion of the motif, for example, a 5-10-5 motif would have a 5′-wing comprising 5 nucleosides, a gap comprising 10 nucleosides, and a 3′-wing comprising 5 nucleosides:

TABLE 5
Certain Gapmer Sugar Motifs
Certain Gapmer Sugar Motifs

| | | | |
|---|---|---|---|
| 2-10-2 | 3-10-2 | 4-10-2 | 5-10-2 |
| 2-10-3 | 3-10-3 | 4-10-3 | 5-10-3 |
| 2-10-4 | 3-10-4 | 4-10-4 | 5-10-4 |
| 2-10-5 | 3-10-5 | 4-10-5 | 5-10-5 |
| 2-9-2 | 3-9-2 | 4-9-2 | 5-9-2 |
| 2-9-3 | 3-9-3 | 4-9-3 | 5-9-3 |
| 2-9-4 | 3-9-4 | 4-9-4 | 5-9-4 |
| 2-9-5 | 3-9-5 | 4-9-5 | 5-9-5 |
| 2-11-2 | 3-11-2 | 4-11-2 | 5-11-2 |
| 2-11-3 | 3-11-3 | 4-11-3 | 5-11-3 |
| 2-11-4 | 3-11-4 | 4-11-4 | 5-11-4 |
| 2-11-5 | 3-11-5 | 4-11-5 | 5-11-5 |
| 2-8-2 | 3-8-2 | 4-8-2 | 5-8-2 |
| 2-8-3 | 3-8-3 | 4-8-3 | 5-8-3 |
| 2-8-4 | 3-8-4 | 4-8-4 | 5-8-4 |
| 2-8-5 | 3-8-5 | 4-8-5 | 5-8-5 |

In certain embodiments, a gapmer comprises a 5′-wing, a gap, and a 3′ wing, wherein the 5′-wing, gap, and 3′ wing are independently selected from among those discussed above. For example, in certain embodiments, a gapmer has a 5′-wing, a gap, and a 3′-wing having features selected from among those listed in the following non-limiting tables:

TABLE 6
Certain Gapmer Nucleoside Motifs

| 5′-wing | Central gap | 3′-wing |
|---|---|---|
| region | region | region |
| ADDA | DDDDDD | ABB |
| ABBA | DDDADDDD | ABAA |
| AAAAAAA | DDDDDDDDDDD | AAA |
| AAAAABB | DDDDDDDD | BBAAAAA |
| ABB | DDDDADDDD | ABB |
| ABB | DDDDBDDDD | BBA |
| ABB | DDDDDDDDD | BBA |
| AABAA | DDDDDDDDD | AABAA |
| ABB | DDDDDD | AABAA |
| AAABAA | DDDDDDDDD | AAABAA |
| AAABAA | DDDDDDDDD | AAB |
| ABAB | DDDDDDDDD | ABAB |
| AAABB | DDDDDDD | BBA |
| ABADB | DDDDDDD | BBA |
| ABA | DBDDDDDDD | BBA |
| ABA | DADDDDDDD | BBA |
| ABAB | DDDDDDDD | BBA |
| AA | DDDDDDDD | BBBBBBBB |
| ABB | DDDDDD | ABADB |
| AAAAB | DDDDDDD | BAAAA |
| ABBB | DDDDDDDDD | AB |
| AB | DDDDDDDDD | BBBA |
| ABBB | DDDDDDDDD | BBBA |
| AB | DDDDDDDD | ABA |
| ABB | DDDDWDDDD | BBA |
| AAABB | DDDWDDD | BBAAA |
| ABB | DDDDWWDDD | BBA |
| ABADB | DDDDDDD | BBA |
| ABBDC | DDDDDDD | BBA |
| ABBDDC | DDDDDD | BBA |
| ABBDCC | DDDDDD | BBA |
| ABB | DWWDWWDWW | BBA |
| ABB | DWDDDDDDD | BBA |
| ABB | DDWDDDDDD | BBA |
| ABB | DWWDDDDDD | BBA |
| AAABB | DDWDDDDDD | AA |
| BB | DDWDWDDDD | BBABBBB |
| ABB | DDDD(^{N}D)DDDD | BBA |
| AAABB | DDD(^{N}D)DDD | BBAAA |
| ABB | DDDD(^{N}D)(^{N}D)DDD | BBA |
| ABB | D(^{N}D)(^{N}D)D(^{N}D)(^{N}D)D(^{N}D)(^{N}D) | BBA |
| ABB | D(^{N}D)DDDDDDD | BBA |
| ABB | DD(^{N}D)DDDDDD | BBA |
| ABB | D(^{N}D)(^{N}D)DDDDDD | BBA |
| AAABB | DD(^{N}D)DDDDDD | AA |
| BB | DD(^{N}D)D(^{N}D)DDDD | BBABBBB |
| ABAB | DDDDDDDDD | BABA |

TABLE 7
Certain Gapmer Nucleoside Motifs

| 5′-wing | Central gap | 3′-wing |
|---|---|---|
| region | region | region |
| ABBW | DDDDDDDD | BBA |
| ABB | DWDDDDDDD | BBA |
| ABB | DDWDDDDDD | BBA |
| ABB | DDDWDDDDD | BBA |
| ABB | DDDDWDDDD | BBA |
| ABB | DDDDDWDDD | BBA |
| ABB | DDDDDDWDD | BBA |
| ABB | DDDDDDDWD | BBA |
| ABB | DDDDDDDD | WBBA |
| ABBWW | DDDDDDD | BBA |
| ABB | DWWDDDDDD | BBA |
| ABB | DDWWDDDDD | BBA |
| ABB | DDDWWDDDD | BBA |
| ABB | DDDDWWDDD | BBA |
| ABB | DDDDDWWDD | BBA |
| ABB | DDDDDDWWD | BBA |
| ABB | DDDDDDD | WWBBA |
| ABBW | DDDDDDD | WBBA |
| ABBW | DDDDDDWD | BBA |
| ABBW | DDDDDWDD | BBA |
| ABBW | DDDDWDDD | BBA |
| ABBW | DDDWDDDD | BBA |
| ABBW | DDWDDDDD | BBA |
| ABBW | DWDDDDDD | BBA |
| ABB | DWDDDDDD | WBBA |
| ABB | DWDDDDDWD | BBA |
| ABB | DWDDDDWDD | BBA |
| ABB | DWDDDWDDD | BBA |
| ABB | DWDDWDDDD | BBA |
| ABB | DWDWDDDDD | BBA |
| ABB | DDWDDDDD | WBBA |
| ABB | DDWDDDDWD | BBA |
| ABB | DDWDDDWDD | BBA |
| ABB | DDWDDWDDD | BBA |
| ABB | DDWDWDDDD | BBA |
| ABB | DDWWDDDDD | BBA |
| ABB | DDDWDDDD | WBBA |
| ABB | DDDWDDDWD | BBA |
| ABB | DDDWDDWDD | BBA |
| ABB | DDDWDWDDD | BBA |
| ABB | DDDWWDDDD | BBA |
| ABB | DDDDWDDD | WBBA |
| ABB | DDDDWDDWD | BBA |
| ABB | DDDDWDWDD | BBA |
| ABB | DDDDWWDDD | BBA |
| ABB | DDDDDWDD | WBBA |
| ABB | DDDDDWDWD | BBA |
| ABB | DDDDDWWDD | BBA |
| ABB | DDDDDDWD | WBBA |

TABLE 8
Certain Gapmer Nucleoside Motifs

| 5′-wing | Central gap | 3′-wing |
|---|---|---|
| region | region | region |
| ABBB | DDDDDDDD | BBA |
| ABB | DBDDDDDDD | BBA |
| ABB | DDBDDDDDD | BBA |
| ABB | DDDBDDDDD | BBA |
| ABB | DDDDBDDDD | BBA |
| ABB | DDDDDBDDD | BBA |
| ABB | DDDDDDBDD | BBA |
| ABB | DDDDDDDBD | BBA |
| ABB | DDDDDDDD | BBBA |
| ABBBB | DDDDDDD | BBA |
| ABB | DBBDDDDDD | BBA |
| ABB | DDBBDDDDD | BBA |
| ABB | DDDBBDDDD | BBA |
| ABB | DDDDBBDDD | BBA |
| ABB | DDDDDBBDD | BBA |
| ABB | DDDDDDBBD | BBA |
| ABB | DDDDDDD | BBBBA |
| ABBB | DDDDDDD | BBBA |
| ABB | DDDDDDBD | BBA |
| ABBB | DDDDDBDD | BBA |
| ABBB | DDDDBDDD | BBA |
| ABBB | DDDBDDDD | BBA |
| ABBB | DDBDDDDD | BBA |
| ABBB | DBDDDDDD | BBA |
| ABB | DBDDDDDD | BBBA |
| ABB | DBDDDDDBD | BBA |
| ABB | DBDDDDBDD | BBA |
| ABB | DBDDDBDDD | BBA |
| ABB | DBDDBDDDD | BBA |
| ABB | DBDBDDDDD | BBA |
| ABB | DDBDDDDD | BBBA |
| ABB | DDBDDDDBD | BBA |
| ABB | DDBDDDBDD | BBA |
| ABB | DDBDDBDDD | BBA |
| ABB | DDBDBDDDD | BBA |
| ABB | DDBBDDDDD | BBA |
| ABB | DDDBDDDD | BBBA |
| ABB | DDDBDDDBD | BBA |
| ABB | DDDBDDBDD | BBA |
| ABB | DDDBDBDDD | BBA |
| ABB | DDDBBDDDD | BBA |
| ABB | DDDDBDDD | BBBA |
| ABB | DDDDBDDBD | BBA |
| ABB | DDDDBDBDD | BBA |
| ABB | DDDDBBDDD | BBA |
| ABB | DDDDDBDD | BBBA |
| ABB | DDDDDBDBD | BBA |
| ABB | DDDDDBBDD | BBA |
| ABB | DDDDDDBD | BBBA |

TABLE 9
Certain Gapmer Nucleoside Motifs

| 5′-wing | Central gap | 3′-wing |
|---|---|---|
| region | region | region |
| ABB | DDDDDDDDD | BBA |
| AB | DBDDDDDDDD | BBA |
| AB | DDBDDDDDDD | BBA |
| AB | DDDBDDDDDD | BBA |
| AB | DDDDBDDDDD | BBA |
| AB | DDDDDBDDDD | BBA |
| AB | DDDDDDBDDD | BBA |
| AB | DDDDDDDBDD | BBA |
| AB | DDDDDDDDBD | BBA |
| AB | DDDDDDDDD | BBBA |
| ABBB | DDDDDDDD | BBA |
| AB | DBBDDDDDDD | BBA |
| AB | DDBBDDDDDD | BBA |
| AB | DDDBBDDDDD | BBA |
| AB | DDDDBBDDDD | BBA |
| AB | DDDDDBBDDD | BBA |
| AB | DDDDDDBBDD | BBA |
| AB | DDDDDDDBBD | BBA |
| AB | DDDDDDDD | BBBBA |
| ABBBB | DDDDDDD | BBA |
| AB | DBBBDDDDDD | BBA |
| AB | DDBBBDDDDD | BBA |
| AB | DDDBBBDDDD | BBA |
| AB | DDDDBBBDDD | BBA |
| AB | DDDDDBBBDD | BBA |
| AB | DDDDDDBBBD | BBA |
| AB | DDDDDDD | BBBBBA |
| AB | DDDDDDDDD | BBBA |
| AB | DDDDDDDBD | BBBA |
| AB | DDDDDBDD | BBBA |
| AB | DDDDBDDD | BBBA |
| AB | DDDBDDDD | BBBA |
| AB | DDBDDDDD | BBBA |
| AB | DBDDDDDD | BBBA |
| AB | DDDDDBD | BBBBA |
| AB | DDDDBDD | BBBBA |
| AB | DDDBDDD | BBBBA |
| AB | DDBDDDD | BBBBA |
| AB | DBDDDDD | BBBBA |
| AB | DDDDBD | BBBBBA |
| AB | DDDBDD | BBBBBA |
| AB | DDBDDD | BBBBBA |
| AB | DBDDDD | BBBBBA |

TABLE 10
Certain Gapmer Nucleoside Motifs

| 5′-wing | Central gap | 3′-wing |
|---|---|---|
| region | region | region |
| AAAAAA | DDDDDDD | BABA |
| AAAAAB | DDDDDDD | BABA |
| AAAABA | DDDDDDD | BABA |
| AAABAA | DDDDDDD | BABA |
| AABAAA | DDDDDDD | BABA |
| ABAAAA | DDDDDDD | BABA |
| BAAAAA | DDDDDDD | BABA |
| ABAAAB | DDDDDDD | BABA |
| ABAABA | DDDDDDD | BABA |
| ABABAA | DDDDDDD | BABA |
| ABBAAA | DDDDDDD | BABA |
| AABAAB | DDDDDDD | BABA |
| AABABA | DDDDDDD | BABA |
| AABBAA | DDDDDDD | BABA |
| AAABAB | DDDDDDD | BABA |
| AAABBA | DDDDDDD | BABA |
| AAAABB | DDDDDDD | BABA |
| BAAAAB | DDDDDDD | BABA |
| BAAABA | DDDDDDD | BABA |
| BAABAA | DDDDDDD | BABA |
| BABAAA | DDDDDDD | BABA |
| BBAAAA | DDDDDDD | BABA |
| BBBAAA | DDDDDDD | BABA |
| BBABAA | DDDDDDD | BABA |
| BBAABA | DDDDDDD | BABA |
| BBAAAB | DDDDDDD | BABA |
| ABABAB | DDDDDDD | BABA |
| BBBBAA | DDDDDDD | BABA |
| BBBABA | DDDDDDD | BABA |
| BBBAAB | DDDDDDD | BABA |
| BBBBBA | DDDDDDD | BABA |
| BBBBAB | DDDDDDD | BABA |
| AAABBB | DDDDDDD | BABA |
| AABABB | DDDDDDD | BABA |
| ABAABB | DDDDDDD | BABA |
| BAAABB | DDDDDDD | BABA |
| AABBBB | DDDDDDD | BABA |
| ABABBB | DDDDDDD | BABA |
| BAABBB | DDDDDDD | BABA |
| ABBBBB | DDDDDDD | BABA |
| BABBBB | DDDDDDD | BABA |
| BBBBBB | DDDDDDD | BABA |

TABLE 11
Certain Gapmer Nucleoside Motifs

| 5′-wing | Central gap | 3′-wing |
|---|---|---|
| region | region | region |
| AAAAA | DDDDDDD | AAAAA |
| AAAAB | DDDDDDD | AAAAA |
| AAABA | DDDDDDD | AAAAA |
| AAABB | DDDDDDD | AAAAA |
| AABAA | DDDDDDD | AAAAA |
| AABAB | DDDDDDD | AAAAA |
| AABBA | DDDDDDD | AAAAA |
| AABBB | DDDDDDD | AAAAA |
| ABAAA | DDDDDDD | AAAAA |
| ABAAB | DDDDDDD | AAAAA |
| ABABA | DDDDDDD | AAAAA |
| ABABB | DDDDDDD | AAAAA |
| ABBAA | DDDDDDD | AAAAA |
| ABBAB | DDDDDDD | AAAAA |
| ABBBA | DDDDDDD | AAAAA |
| ABBBB | DDDDDDD | AAAAA |
| BAAAA | DDDDDDD | AAAAA |
| BAAAB | DDDDDDD | AAAAA |
| BAABA | DDDDDDD | AAAAA |
| BAABB | DDDDDDD | AAAAA |
| BABAA | DDDDDDD | AAAAA |
| BABAB | DDDDDDD | AAAAA |
| BABBA | DDDDDDD | AAAAA |
| BABBB | DDDDDDD | AAAAA |
| BBAAA | DDDDDDD | AAAAA |
| BBAAB | DDDDDDD | AAAAA |
| BBABA | DDDDDDD | AAAAA |
| BBABB | DDDDDDD | AAAAA |
| BBBAA | DDDDDDD | AAAAA |
| BBBAB | DDDDDDD | AAAAA |
| BBBBA | DDDDDDD | AAAAA |
| BBBBB | DDDDDDD | AAAAA |
| AAAAA | DDDDDDD | BAAAA |
| AAAAB | DDDDDDD | BAAAA |
| AAABA | DDDDDDD | BAAAA |
| AAABB | DDDDDDD | BAAAA |
| AABAA | DDDDDDD | BAAAA |
| AABAB | DDDDDDD | BAAAA |
| AABBA | DDDDDDD | BAAAA |
| AABBB | DDDDDDD | BAAAA |
| ABAAA | DDDDDDD | BAAAA |
| ABAAB | DDDDDDD | BAAAA |
| ABABA | DDDDDDD | BAAAA |
| ABABB | DDDDDDD | BAAAA |
| ABBAA | DDDDDDD | BAAAA |
| ABBAB | DDDDDDD | BAAAA |
| ABBBA | DDDDDDD | BAAAA |
| ABBBB | DDDDDDD | BAAAA |
| BAAAA | DDDDDDD | BAAAA |
| BAAAB | DDDDDDD | BAAAA |
| BAABA | DDDDDDD | BAAAA |
| BAABB | DDDDDDD | BAAAA |
| BABAA | DDDDDDD | BAAAA |
| BABAB | DDDDDDD | BAAAA |
| BABBA | DDDDDDD | BAAAA |
| BABBB | DDDDDDD | BAAAA |
| BBAAA | DDDDDDD | BAAAA |
| BBAAB | DDDDDDD | BAAAA |
| BBABA | DDDDDDD | BAAAA |
| BBABB | DDDDDDD | BAAAA |
| BBBAA | DDDDDDD | BAAAA |
| BBBAB | DDDDDDD | BAAAA |
| BBBBA | DDDDDDD | BAAAA |
| BBBBB | DDDDDDD | BAAAA |
| AAAAA | DDDDDDD | BBAAA |
| AAAAB | DDDDDDD | BBAAA |
| AAABA | DDDDDDD | BBAAA |
| AAABB | DDDDDDD | BBAAA |
| AABAA | DDDDDDD | BBAAA |
| AABAB | DDDDDDD | BBAAA |
| AABBA | DDDDDDD | BBAAA |
| AABBB | DDDDDDD | BBAAA |
| ABAAA | DDDDDDD | BBAAA |
| ABAAB | DDDDDDD | BBAAA |
| ABABA | DDDDDDD | BBAAA |
| ABABB | DDDDDDD | BBAAA |
| ABBAA | DDDDDDD | BBAAA |
| ABBAB | DDDDDDD | BBAAA |
| ABBBA | DDDDDDD | BBAAA |
| ABBBB | DDDDDDD | BBAAA |
| BAAAA | DDDDDDD | BBAAA |
| BAAAB | DDDDDDD | BBAAA |
| BAABA | DDDDDDD | BBAAA |
| BAABB | DDDDDDD | BBAAA |
| BABAA | DDDDDDD | BBAAA |
| BABAB | DDDDDDD | BBAAA |
| BABBA | DDDDDDD | BBAAA |
| BABBB | DDDDDDD | BBAAA |
| BBAAA | DDDDDDD | BBAAA |
| BBAAB | DDDDDDD | BBAAA |
| BBABA | DDDDDDD | BBAAA |
| BBABB | DDDDDDD | BBAAA |
| BBBAA | DDDDDDD | BBAAA |
| BBBAB | DDDDDDD | BBAAA |
| BBBBA | DDDDDDD | BBAAA |
| BBBBB | DDDDDDD | BBAAA |
| AAAAA | DDDDDDD | BBBAA |
| AAAAB | DDDDDDD | BBBAA |
| AAABA | DDDDDDD | BBBAA |
| AAABB | DDDDDDD | BBBAA |
| AABAA | DDDDDDD | BBBAA |
| AABAB | DDDDDDD | BBBAA |
| AABBA | DDDDDDD | BBBAA |
| AABBB | DDDDDDD | BBBAA |
| ABAAA | DDDDDDD | BBBAA |
| ABAAB | DDDDDDD | BBBAA |
| ABABA | DDDDDDD | BBBAA |
| ABABB | DDDDDDD | BBBAA |
| ABBAA | DDDDDDD | BBBAA |
| ABBAB | DDDDDDD | BBBAA |
| ABBBA | DDDDDDD | BBBAA |
| ABBBB | DDDDDDD | BBBAA |
| BAAAA | DDDDDDD | BBBAA |
| BAAAB | DDDDDDD | BBBAA |
| BAABA | DDDDDDD | BBBAA |
| BAABB | DDDDDDD | BBBAA |
| BABAA | DDDDDDD | BBBAA |
| BABAB | DDDDDDD | BBBAA |
| BABBA | DDDDDDD | BBBAA |
| BABBB | DDDDDDD | BBBAA |
| BBAAA | DDDDDDD | BBBAA |
| BBAAB | DDDDDDD | BBBAA |
| BBABA | DDDDDDD | BBBAA |
| BBABB | DDDDDDD | BBBAA |
| BBBAA | DDDDDDD | BBBAA |
| BBBAB | DDDDDDD | BBBAA |
| BBBBA | DDDDDDD | BBBAA |
| BBBBB | DDDDDDD | BBBAA |
| AAAAA | DDDDDDD | BBBBA |
| AAAAB | DDDDDDD | BBBBA |
| AAABA | DDDDDDD | BBBBA |
| AAABB | DDDDDDD | BBBBA |
| AABAA | DDDDDDD | BBBBA |
| AABAB | DDDDDDD | BBBBA |
| AABBA | DDDDDDD | BBBBA |
| AABBB | DDDDDDD | BBBBA |
| ABAAA | DDDDDDD | BBBBA |
| ABAAB | DDDDDDD | BBBBA |
| ABABA | DDDDDDD | BBBBA |
| ABABB | DDDDDDD | BBBBA |
| ABBAA | DDDDDDD | BBBBA |
| ABBAB | DDDDDDD | BBBBA |
| ABBBA | DDDDDDD | BBBBA |
| ABBBB | DDDDDDD | BBBBA |
| BAAAA | DDDDDDD | BBBBA |
| BAAAB | DDDDDDD | BBBBA |
| BAABA | DDDDDDD | BBBBA |
| BAABB | DDDDDDD | BBBBA |
| BABAA | DDDDDDD | BBBBA |
| BABAB | DDDDDDD | BBBBA |
| BABBA | DDDDDDD | BBBBA |
| BABBB | DDDDDDD | BBBBA |
| BBAAA | DDDDDDD | BBBBA |
| BBAAB | DDDDDDD | BBBBA |
| BBABA | DDDDDDD | BBBBA |
| BBABB | DDDDDDD | BBBBA |
| BBBAA | DDDDDDD | BBBBA |
| BBBAB | DDDDDDD | BBBBA |
| BBBBA | DDDDDDD | BBBBA |
| BBBBB | DDDDDDD | BBBBA |
| AAAAA | DDDDDDD | BBBBB |
| AAAAB | DDDDDDD | BBBBB |
| AAABA | DDDDDDD | BBBBB |
| AAABB | DDDDDDD | BBBBB |
| AABAA | DDDDDDD | BBBBB |
| AABAB | DDDDDDD | BBBBB |
| AABBA | DDDDDDD | BBBBB |
| AABBB | DDDDDDD | BBBBB |
| ABAAA | DDDDDDD | BBBBB |
| ABAAB | DDDDDDD | BBBBB |
| ABABA | DDDDDDD | BBBBB |
| ABABB | DDDDDDD | BBBBB |
| ABBAA | DDDDDDD | BBBBB |
| ABBAB | DDDDDDD | BBBBB |
| ABBBA | DDDDDDD | BBBBB |
| ABBBB | DDDDDDD | BBBBB |
| BAAAA | DDDDDDD | BBBBB |
| BAAAB | DDDDDDD | BBBBB |
| BAABA | DDDDDDD | BBBBB |
| BAABB | DDDDDDD | BBBBB |
| BABAA | DDDDDDD | BBBBB |
| BABAB | DDDDDDD | BBBBB |
| BABBA | DDDDDDD | BBBBB |
| BABBB | DDDDDDD | BBBBB |
| BBAAA | DDDDDDD | BBBBB |
| BBAAB | DDDDDDD | BBBBB |
| BBABA | DDDDDDD | BBBBB |
| BBABB | DDDDDDD | BBBBB |
| BBBAA | DDDDDDD | BBBBB |
| BBBAB | DDDDDDD | BBBBB |
| BBBBA | DDDDDDD | BBBBB |
| BBBBB | DDDDDDD | BBBBB |

TABLE 12
Certain Gapmer Nucleoside Motifs

| 5′-wing | Central gap | 3′-wing |
|---|---|---|
| region | region | region |
| AAAW | DDDDDDDD | BBA |
| AABW | DDDDDDDD | BBA |
| ABAW | DDDDDDDD | BBA |
| ABBW | DDDDDDDD | BBA |
| BAAW | DDDDDDDD | BBA |
| BABW | DDDDDDDD | BBA |
| BBAW | DDDDDDDD | BBA |
| BBBW | DDDDDDDD | BBA |
| ABB | DDDDDDDD | WAAA |
| ABB | DDDDDDDD | WAAB |
| ABB | DDDDDDDD | WABA |
| ABB | DDDDDDDD | WABB |
| ABB | DDDDDDDD | WBAA |
| ABB | DDDDDDDD | WBAB |
| ABB | DDDDDDDD | WBBA |
| ABB | DDDDDDDD | WBBB |
| AAAWW | DDDDDDD | BBA |
| AABWW | DDDDDDD | BBA |
| ABAWW | DDDDDDD | BBA |
| ABBWW | DDDDDDD | BBA |
| BAAWW | DDDDDDD | BBA |
| BABWW | DDDDDDD | BBA |
| BBAWW | DDDDDDD | BBA |
| BBBWW | DDDDDDD | BBA |
| ABB | DDDDDDD | WWAAA |
| ABB | DDDDDDD | WWAAB |
| ABB | DDDDDDD | WWABA |
| ABB | DDDDDDD | WWABB |
| ABB | DDDDDDD | WWBAA |
| ABB | DDDDDDD | WWBAB |
| ABB | DDDDDDD | WWBBA |
| ABB | DDDDDDD | WWBBB |
| AAAAW | DDDDDDD | BBA |
| AAABW | DDDDDDD | BBA |
| AABAW | DDDDDDD | BBA |
| AABBW | DDDDDDD | BBA |
| ABAAW | DDDDDDD | BBA |
| ABABW | DDDDDDD | BBA |
| ABBAW | DDDDDDD | BBA |
| ABBBW | DDDDDDD | BBA |
| BAAAW | DDDDDDD | BBA |
| BAABW | DDDDDDD | BBA |
| BABAW | DDDDDDD | BBA |
| BABBW | DDDDDDD | BBA |
| BBAAW | DDDDDDD | BBA |
| BBABW | DDDDDDD | BBA |
| BBBAW | DDDDDDD | BBA |
| BBBBW | DDDDDDD | WAAAA |
| ABB | DDDDDDD | WAAAB |
| ABB | DDDDDDD | WAABA |
| ABB | DDDDDDD | WAABB |
| ABB | DDDDDDD | WABAA |
| ABB | DDDDDDD | WABAB |
| ABB | DDDDDDD | WABBA |
| ABB | DDDDDDD | WABBB |
| ABB | DDDDDDD | WBAAA |
| ABB | DDDDDDD | WBAAB |
| ABB | DDDDDDD | WBABA |
| ABB | DDDDDDD | WBABB |
| ABB | DDDDDDD | WBBAA |
| ABB | DDDDDDD | WBBAB |
| ABB | DDDDDDD | WBBBA |
| ABB | DDDDDDD | WBBBB |

wherein each A is a modified nucleoside of a first type, each B is a modified nucleoside of a second type and each W is a modified nucleoside or nucleobase of either the first type, the second type or a third type, each D is a nucleoside comprising an unmodified 2′deoxy sugar moiety and unmodified nucleobase, and ^{N}D is modified nucleoside comprising a modified nucleobase and an unmodified 2′deoxy sugar moiety.

In certain embodiments, each A comprises a modified sugar moiety. In certain embodiments, each A comprises a 2′-substituted sugar moiety. In certain embodiments, each A comprises a 2′-substituted sugar moiety selected from among F, ara-F, OCH₃ and O(CH₂)₂—OCH₃. In certain embodiments, each A comprises a bicyclic sugar moiety. In certain embodiments, each A comprises a bicyclic sugar moiety selected from among cEt, cMOE, LNA, α-L-LNA, ENA and 2′-thio LNA. In certain embodiments, each A comprises a modified nucleobase. In certain embodiments, each A comprises a modified nucleobase selected from among 2-thio-thymidine nucleoside and 5-propyne uridine nucleoside. In certain embodiments, each A comprises an HNA. In certain embodiments, each A comprises an F-HNA. In certain embodiments, each A comprises a 5′-substituted sugar moiety selected from among 5′-Me, and 5′-(R)-Me.

In certain embodiments, each B comprises a modified sugar moiety. In certain embodiments, each B comprises a 2′-substituted sugar moiety. In certain embodiments, each B comprises a 2′-substituted sugar moiety selected from among F, (ara)-F, OCH₃ and O(CH₂)₂—OCH₃. In certain embodiments, each B comprises a bicyclic sugar moiety. In certain embodiments, each B comprises a bicyclic sugar moiety selected from among cEt, cMOE, LNA, α-L-LNA, ENA and 2′-thio LNA. In certain embodiments, each B comprises a modified nucleobase. In certain embodiments, each B comprises a modified nucleobase selected from among 2-thio-thymidine nucleoside and 5-propyne uridine nucleoside. In certain embodiments, each B comprises an HNA. In certain embodiments, each B comprises an F-HNA. In certain embodiments, each B comprises a 5′-substituted sugar moiety selected from among 5′-Me, and 5′-(R)-Me.

In certain embodiments, each C comprises a modified sugar moiety. In certain embodiments, each C comprises a 2′-substituted sugar moiety. In certain embodiments, each C comprises a 2′-substituted sugar moiety selected from among F, (ara)-F, OCH₃ and O(CH₂)₂—OCH₃. In certain embodiments, each C comprises a 5′-substituted sugar moiety. In certain embodiments, each C comprises a 5′-substituted sugar moiety selected from among 5′-Me, and 5′-(R)-Me. In certain embodiments, each C comprises a bicyclic sugar moiety. In certain embodiments, each C comprises a bicyclic sugar moiety selected from among cEt, cMOE, LNA, α-L-LNA, ENA and 2′-thio LNA. In certain embodiments, each C comprises a modified nucleobase. In certain embodiments, each C comprises a modified nucleobase selected from among 2-thio-thymidine and 5-propyne uridine. In certain embodiments, each C comprises a 2-thio-thymidine nucleoside. In certain embodiments, each C comprises an HNA. In certain embodiments, each C comprises an F-HNA.

In certain embodiments, each W comprises a modified sugar moiety. In certain embodiments, each W comprises a 2′-substituted sugar moiety. In certain embodiments, each W comprises a 2′-substituted sugar moiety selected from among F, (ara)-F, OCH₃ and O(CH₂)₂—OCH₃. In certain embodiments, each W comprises a 5′-substituted sugar moiety. In certain embodiments, each W comprises a 5′-substituted sugar moiety selected from among 5′-Me, and 5′-(R)-Me. In certain embodiments, each W comprises a bicyclic sugar moiety. In certain embodiments, each W comprises a bicyclic sugar moiety selected from among cEt, cMOE, LNA, α-L-LNA, ENA and 2′-thio LNA. In certain embodiments, each W comprises a sugar surrogate. In certain embodiments, each W comprises a sugar surrogate selected from among HNA and F-HNA. In certain embodiments, each W comprises a 2-thio-thymidine nucleoside.

In certain embodiments, at least one of A or B comprises a bicyclic sugar moiety, and the other comprises a 2′-substituted sugar moiety. In certain embodiments, one of A or B is an LNA nucleoside and the other of A or B comprises a 2′-substituted sugar moiety. In certain embodiments, one of A or B is a cEt nucleoside and the other of A or B comprises a 2′-substituted sugar moiety. In certain embodiments, one of A or B is an α-L-LNA nucleoside and the other of A or B comprises a 2′-substituted sugar moiety. In certain embodiments, one of A or B is an LNA nucleoside and the other of A or B comprises a 2′-MOE sugar moiety. In certain embodiments, one of A or B is a cEt nucleoside and the other of A or B comprises a 2′-MOE sugar moiety. In certain embodiments, one of A or B is an α-L-LNA nucleoside and the other of A or B comprises a 2′-MOE sugar moiety. In certain embodiments, one of A or B is an LNA nucleoside and the other of A or B comprises a 2′-F sugar moiety. In certain embodiments, one of A or B is a cEt nucleoside and the other of A or B comprises a 2′-F sugar moiety. In certain embodiments, one of A or B is an α-L-LNA nucleoside and the other of A or B comprises a 2′-F sugar moiety. In certain embodiments, one of A or B is an LNA nucleoside and the other of A or B comprises a 2′-(ara)-F sugar moiety. In certain embodiments, one of A or B is a cEt nucleoside and the other of A or B comprises a 2′-(ara)-F sugar moiety. In certain embodiments, one of A or B is an α-L-LNA nucleoside and the other of A or B comprises a 2′-(ara)-F sugar moiety.

In certain embodiments, A comprises a bicyclic sugar moiety, and B comprises a 2′-substituted sugar moiety. In certain embodiments, A is an LNA nucleoside and B comprises a 2′-substituted sugar moiety. In certain embodiments, A is a cEt nucleoside and B comprises a 2′-substituted sugar moiety. In certain embodiments, A is an α-L-LNA nucleoside and B comprises a 2′-substituted sugar moiety.

In certain embodiments, A comprises a bicyclic sugar moiety, and B comprises a 2′-MOE sugar moiety. In certain embodiments, A is an LNA nucleoside and B comprises a 2′-MOE sugar moiety. In certain embodiments, A is a cEt nucleoside and B comprises a 2′-MOE sugar moiety. In certain embodiments, A is an α-L-LNA nucleoside and B comprises a 2′-MOE sugar moiety.

In certain embodiments, A comprises a bicyclic sugar moiety, and B comprises a 2′-F sugar moiety. In certain embodiments, A is an LNA nucleoside and B comprises a 2′-F sugar moiety. In certain embodiments, A is a cEt nucleoside and B comprises a 2′-F sugar moiety. In certain embodiments, A is an α-L-LNA nucleoside and B comprises a 2′-F sugar moiety.

In certain embodiments, A comprises a bicyclic sugar moiety, and B comprises a 2′-(ara)-F sugar moiety. In certain embodiments, A is an LNA nucleoside and B comprises a 2′-(ara)-F sugar moiety. In certain embodiments, A is a cEt nucleoside and B comprises a 2′-(ara)-F sugar moiety. In certain embodiments, A is an α-L-LNA nucleoside and B comprises a 2′-(ara)-F sugar moiety.

In certain embodiments, B comprises a bicyclic sugar moiety, and A comprises a 2′-MOE sugar moiety. In certain embodiments, B is an LNA nucleoside and A comprises a 2′-MOE sugar moiety. In certain embodiments, B is a cEt nucleoside and A comprises a 2′-MOE sugar moiety. In certain embodiments, B is an α-L-LNA nucleoside and A comprises a 2′-MOE sugar moiety.

In certain embodiments, B comprises a bicyclic sugar moiety, and A comprises a 2′-F sugar moiety. In certain embodiments, B is an LNA nucleoside and A comprises a 2′-F sugar moiety. In certain embodiments, B is a cEt nucleoside and A comprises a 2′-F sugar moiety. In certain embodiments, B is an α-L-LNA nucleoside and A comprises a 2′-F sugar moiety.

In certain embodiments, B comprises a bicyclic sugar moiety, and A comprises a 2′-(ara)-F sugar moiety. In certain embodiments, B is an LNA nucleoside and A comprises a 2′-(ara)-F sugar moiety. In certain embodiments, B is a cEt nucleoside and A comprises a 2′-(ara)-F sugar moiety. In certain embodiments, B is an α-L-LNA nucleoside and A comprises a 2′-(ara)-F sugar moiety.

In certain embodiments, at least one of A or B comprises a bicyclic sugar moiety, another of A or B comprises a 2′-substituted sugar moiety and W comprises a modified nucleobase. In certain embodiments, one of A or B is an LNA nucleoside, another of A or B comprises a 2′-substituted sugar moiety, and W comprises a modified nucleobase. In certain embodiments, one of A or B is a cEt nucleoside, another of A or B comprises a 2′-substituted sugar moiety, and C comprises a modified nucleobase. In certain embodiments, one of A or B is an α-L-LNA nucleoside, another of A or B comprises a 2′-substituted sugar moiety, and W comprises a modified nucleobase.

In certain embodiments, one of A or B comprises a bicyclic sugar moiety, another of A or B comprises a 2′-MOE sugar moiety, and W comprises a modified nucleobase. In certain embodiments, one of A or B is an LNA nucleoside, another of A or B comprises a 2′-MOE sugar moiety, and W comprises a modified nucleobase. In certain embodiments, one of A or B is a cEt nucleoside, another of A or B comprises a 2′-MOE sugar moiety, and W comprises a modified nucleobase. In certain embodiments, one of A or B is an α-L-LNA nucleoside, another of A or B comprises a 2′-MOE sugar moiety, and W comprises a modified nucleobase.

In certain embodiments, one of A or B comprises a bicyclic sugar moiety, another of A or B comprises a 2′-F sugar moiety, and W comprises a modified nucleobase. In certain embodiments, one of A or B is an LNA nucleoside, another of A or B comprises a 2′-F sugar moiety, and W comprises a modified nucleobase. In certain embodiments, one of A or B is a cEt nucleoside, another of A or B comprises a 2′-F sugar moiety, and W comprises a modified nucleobase. In certain embodiments, one of A or B is an α-L-LNA nucleoside, another of A or B comprises a 2′-F sugar moiety, and W comprises a modified nucleobase.

In certain embodiments, one of A or B comprises a bicyclic sugar moiety, another of A or B comprises a 2′-(ara)-F sugar moiety, and W comprises a modified nucleobase. In certain embodiments, one of A or B is an LNA nucleoside, another of A or B comprises a 2′-(ara)-F sugar moiety, and W comprises a modified nucleobase. In certain embodiments, one of A or B is a cEt nucleoside, another of A or B comprises a 2′-(ara)-F sugar moiety, and W comprises a modified nucleobase. In certain embodiments, one of A or B is an α-L-LNA nucleoside, another of A or B comprises a 2′-(ara)-F sugar moiety, and W comprises a modified nucleobase.

In certain embodiments, one of A or B comprises a bicyclic sugar moiety, another of A or B comprises a 2′-substituted sugar moiety, and W comprises a 2-thio-thymidine nucleobase. In certain embodiments, one of A or B is an LNA nucleoside, another of A or B comprises a 2′-substituted sugar moiety, and W comprises a 2-thio-thymidine nucleobase. In certain embodiments, one of A or B is a cEt nucleoside, another of A or B comprises a 2′-substituted sugar moiety, and W comprises a 2-thio-thymidine nucleobase. In certain embodiments, one of A or B is an α-L-LNA nucleoside, another of A or B comprises a 2′-substituted sugar moiety, and W comprises a 2-thio-thymidine nucleobase.

In certain embodiments, one of A or B comprises a bicyclic sugar moiety, another of A or B comprises a 2′-MOE sugar moiety, and W comprises a 2-thio-thymidine nucleobase. In certain embodiments, one of A or B is an LNA nucleoside, another of A or B comprises a 2′-MOE sugar moiety, and W comprises a 2-thio-thymidine nucleobase. In certain embodiments, one of A or B is a cEt nucleoside, another of A or B comprises a 2′-MOE sugar moiety, and W comprises a 2-thio-thymidine nucleobase. In certain embodiments, one of A or B is an α-L-LNA nucleoside, another of A or B comprises a 2′-MOE sugar moiety, and W comprises a 2-thio-thymidine nucleobase.

In certain embodiments, one of A or B comprises a bicyclic sugar moiety, another of A or B comprises a 2′-F sugar moiety, and W comprises a 2-thio-thymidine nucleobase. In certain embodiments, one of A or B is an LNA nucleoside, another of A or B comprises a 2′-F sugar moiety, and W comprises a 2-thio-thymidine nucleobase. In certain embodiments, one of A or B is a cEt nucleoside, another of A or B comprises a 2′-F sugar moiety, and W comprises a 2-thio-thymidine nucleobase. In certain embodiments, one of A or B is an α-L-LNA nucleoside, another of A or B comprises a 2′-F sugar moiety, and W comprises a 2-thio-thymidine nucleobase.

In certain embodiments, one of A or B comprises a bicyclic sugar moiety, another of A or B comprises a 2′-(ara)-F sugar moiety, and W comprises a 2-thio-thymidine nucleobase. In certain embodiments, one of A or B is an LNA nucleoside, another of A or B comprises a 2′-(ara)-F sugar moiety, and W comprises a 2-thio-thymidine nucleobase. In certain embodiments, one of A or B is a cEt nucleoside, another of A or B comprises a 2′-(ara)-F sugar moiety, and W comprises a 2-thio-thymidine nucleobase. In certain embodiments, one of A or B is an α-L-LNA nucleoside, another of A or B comprises a 2′-(ara)-F sugar moiety, and W comprises 2-thio-thymidine nucleobase.

In certain embodiments, one of A or B comprises a bicyclic sugar moiety, another of A or B comprises a 2′-MOE sugar moiety, and W comprises a 5-propyne uridine nucleobase. In certain embodiments, one of A or B is an LNA nucleoside, another of A or B comprises a 2′-MOE sugar moiety, and C comprises a 5-propyne uridine nucleobase. In certain embodiments, one of A or B is a cEt nucleoside, another of A or B comprises a 2′-MOE sugar moiety, and W comprises a 5-propyne uridine nucleobase. In certain embodiments, one of A or B is an α-L-LNA nucleoside, another of A or B comprises a 2′-MOE sugar moiety, and C comprises a 5-propyne uridine nucleobase.

In certain embodiments, one of A or B comprises a bicyclic sugar moiety, another of A or B comprises a 2′-F sugar moiety, and W comprises a 5-propyne uridine nucleobase. In certain embodiments, one of A or B is an LNA nucleoside, another of A or B comprises a 2′-F sugar moiety, and C comprises a 5-propyne uridine nucleobase. In certain embodiments, one of A or B is a cEt nucleoside, another of A or B comprises a 2′-F sugar moiety, and W comprises a 5-propyne uridine nucleobase. In certain embodiments, one of A or B is an α-L-LNA nucleoside, another of A or B comprises a 2′-F sugar moiety, and W comprises a 5-propyne uridine nucleobase.

In certain embodiments, one of A or B comprises a bicyclic sugar moiety, another of A or B comprises a 2′-(ara)-F sugar moiety, and W comprises a 5-propyne uridine nucleobase. In certain embodiments, one of A or B is an LNA nucleoside, another of A or B comprises a 2′-(ara)-F sugar moiety, and W comprises a 5-propyne uridine nucleobase. In certain embodiments, one of A or B is a cEt nucleoside, another of A or B comprises a 2′-(ara)-F sugar moiety, and W comprises a 5-propyne uridine nucleobase. In certain embodiments, one of A or B is an α-L-LNA nucleoside, another of A or B comprises a 2′-(ara)-F sugar moiety, and W comprises a 5-propyne uridine nucleobase.

In certain embodiments, one of A or B comprises a bicyclic sugar moiety, another of A or B comprises a 2′-MOE sugar moiety, and W comprises a sugar surrogate. In certain embodiments, one of A or B is an LNA nucleoside, another of A or B comprises a 2′-MOE sugar moiety, and W comprises a sugar surrogate. In certain embodiments, one of A or B is a cEt nucleoside, another of A or B comprises a 2′-MOE sugar moiety, and W comprises a sugar surrogate. In certain embodiments, one of A or B is an α-L-LNA nucleoside, another of A or B comprises a 2′-MOE sugar moiety, and W comprises a sugar surrogate.

In certain embodiments, one of A or B comprises a bicyclic sugar moiety, another of A or B comprises a 2′-F sugar moiety, and W comprises a sugar surrogate. In certain embodiments, one of A or B is an LNA nucleoside, another of A or B comprises a 2′-F sugar moiety, and W comprises a sugar surrogate. In certain embodiments, one of A or B is a cEt nucleoside, another of A or B comprises a 2′-F sugar moiety, and W comprises a sugar surrogate. In certain embodiments, one of A or B is an α-L-LNA nucleoside, another of A or B comprises a 2′-F sugar moiety, and W comprises a sugar surrogate.

In certain embodiments, one of A or B comprises a bicyclic sugar moiety, another of A or B comprises a 2′-(ara)-F sugar moiety, and W comprises a sugar surrogate. In certain embodiments, one of A or B is an LNA nucleoside, another of A or B comprises a 2′-(ara)-F sugar moiety, and W comprises a sugar surrogate. In certain embodiments, one of A or B is a cEt nucleoside, another of A or B comprises a 2′-(ara)-F sugar moiety, and W comprises a sugar surrogate. In certain embodiments, one of A or B is an α-L-LNA nucleoside, another of A or B comprises a 2′-(ara)-F sugar moiety, and W comprises sugar surrogate.

In certain embodiments, one of A or B comprises a bicyclic sugar moiety, another of A or B comprises a 2′-MOE sugar moiety, and W comprises a HNA sugar surrogate. In certain embodiments, one of A or B is an LNA nucleoside, another of A or B comprises a 2′-MOE sugar moiety, and W comprises a HNA sugar surrogate. In certain embodiments, one of A or B is a cEt nucleoside, another of A or B comprises a 2′-MOE sugar moiety, and W comprises a HNA sugar surrogate. In certain embodiments, one of A or B is an α-L-LNA nucleoside, another of A or B comprises a 2′-MOE sugar moiety, and W comprises a HNA sugar surrogate.

In certain embodiments, one of A or B comprises a bicyclic sugar moiety, another of A or B comprises a 2′-F sugar moiety, and W comprises a HNA sugar surrogate. In certain embodiments, one of A or B is an LNA nucleoside, another of A or B comprises a 2′-F sugar moiety, and W comprises a HNA sugar surrogate. In certain embodiments, one of A or B is a cEt nucleoside, another of A or B comprises a 2′-F sugar moiety, and W comprises a HNA sugar surrogate. In certain embodiments, one of A or B is an α-L-LNA nucleoside, another of A or B comprises a 2′-F sugar moiety, and W comprises a sugar HNA surrogate.

In certain embodiments, one of A or B comprises a bicyclic sugar moiety, another of A or B comprises a 2′-(ara)-F sugar moiety, and W comprises a HNA sugar surrogate. In certain embodiments, one of A or B is an LNA nucleoside, another of A or B comprises a 2′-(ara)-F sugar moiety, and W comprises a HNA sugar surrogate. In certain embodiments, one of A or B is a cEt nucleoside, another of A or B comprises a 2′-(ara)-F sugar moiety, and W comprises a HNA sugar surrogate. In certain embodiments, one of A or B is an α-L-LNA nucleoside, another of A or B comprises a 2′-(ara)-F sugar moiety, and W comprises a HNA sugar surrogate.

In certain embodiments, one of A or B comprises a bicyclic sugar moiety, another of A or B comprises a 2′-MOE sugar moiety, and W comprises a F-HNA sugar surrogate. In certain embodiments, one of A or B is an LNA nucleoside, another of A or B comprises a 2′-MOE sugar moiety, and W comprises a F-HNA sugar surrogate. In certain embodiments, one of A or B is a cEt nucleoside, another of A or B comprises a 2′-MOE sugar moiety, and W comprises a F-HNA sugar surrogate. In certain embodiments, one of A or B is an α-L-LNA nucleoside, another of A or B comprises a 2′-MOE sugar moiety, and W comprises a F-HNA sugar surrogate.

In certain embodiments, one of A or B comprises a bicyclic sugar moiety, another of A or B comprises a 2′-F sugar moiety, and W comprises a F-HNA sugar surrogate. In certain embodiments, one of A or B is an LNA nucleoside, another of A or B comprises a 2′-F sugar moiety, and W comprises a F-HNA sugar surrogate. In certain embodiments, one of A or B is a cEt nucleoside, another of A or B comprises a 2′-F sugar moiety, and W comprises a F-HNA sugar surrogate. In certain embodiments, one of A or B is an α-L-LNA nucleoside, another of A or B comprises a 2′-F sugar moiety, and W comprises a F-HNA sugar surrogate.

In certain embodiments, one of A or B comprises a bicyclic sugar moiety, another of A or B comprises a 2′-(ara)-F sugar moiety, and W comprises a F-HNA sugar surrogate. In certain embodiments, one of A or B is an LNA nucleoside, another of A or B comprises a 2′-(ara)-F sugar moiety, and W comprises a F-HNA sugar surrogate. In certain embodiments, one of A or B is a cEt nucleoside, another of A or B comprises a 2′-(ara)-F sugar moiety, and W comprises a F-HNA sugar surrogate. In certain embodiments, one of A or B is an α-L-LNA nucleoside, another of A or B comprises a 2′-(ara)-F sugar moiety, and W comprises a F-HNA sugar surrogate.

In certain embodiments, one of A or B comprises a bicyclic sugar moiety, another of A or B comprises a 2′-MOE sugar moiety, and W comprises a 5′-Me DNA sugar moiety. In certain embodiments, one of A or B is an LNA nucleoside, another of A or B comprises a 2′-MOE sugar moiety, and W comprises a 5′-Me DNA sugar moiety. In certain embodiments, one of A or B is a cEt nucleoside, another of A or B comprises a 2′-MOE sugar moiety, and W comprises a 5′-Me DNA sugar moiety. In certain embodiments, one of A or B is an α-L-LNA nucleoside, another of A or B comprises a 2′-MOE sugar moiety, and W comprises a 5′-Me DNA sugar moiety.

In certain embodiments, one of A or B comprises a bicyclic sugar moiety, another of A or B comprises a 2′-F sugar moiety, and W comprises a 5′-Me DNA sugar moiety. In certain embodiments, one of A or B is an LNA nucleoside, another of A or B comprises a 2′-F sugar moiety, and W comprises a 5′-Me DNA sugar moiety. In certain embodiments, one of A or B is a cEt nucleoside, another of A or B comprises a 2′-F sugar moiety, and W comprises a 5′-Me DNA sugar moiety. In certain embodiments, one of A or B is an α-L-LNA nucleoside, another of A or B comprises a 2′-F sugar moiety, and W comprises a 5′-Me DNA sugar moiety.

In certain embodiments, one of A or B comprises a bicyclic sugar moiety, another of A or B comprises a 2′-(ara)-F sugar moiety, and W comprises a 5′-Me DNA sugar moiety. In certain embodiments, one of A or B is an LNA nucleoside, another of A or B comprises a 2′-(ara)-F sugar moiety, and W comprises a 5′-Me DNA sugar moiety. In certain embodiments, one of A or B is a cEt nucleoside, another of A or B comprises a 2′-(ara)-F sugar moiety, and W comprises a 5′-Me DNA sugar moiety. In certain embodiments, one of A or B is an α-L-LNA nucleoside, another of A or B comprises a 2′-(ara)-F sugar moiety, and W comprises a 5′-Me DNA sugar moiety.

In certain embodiments, one of A or B comprises a bicyclic sugar moiety, another of A or B comprises a 2′-MOE sugar moiety, and W comprises a 5′-(R)-Me DNA sugar moiety. In certain embodiments, one of A or B is an LNA nucleoside, another of A or B comprises a 2′-MOE sugar moiety, and W comprises a 5′-(R)-Me DNA sugar moiety. In certain embodiments, one of A or B is a cEt nucleoside, another of A or B comprises a 2′-MOE sugar moiety, and W comprises a 5′-(R)-Me DNA sugar moiety. In certain embodiments, one of A or B is an α-L-LNA nucleoside, another of A or B comprises a 2′-MOE sugar moiety, and W comprises a 5′-(R)-Me DNA sugar moiety.

In certain embodiments, one of A or B comprises a bicyclic sugar moiety, another of A or B comprises a 2′-F sugar moiety, and W comprises a 5′-(R)-Me DNA sugar moiety. In certain embodiments, one of A or B is an LNA nucleoside, another of A or B comprises a 2′-F sugar moiety, and W comprises a 5′-(R)-Me DNA sugar moiety. In certain embodiments, one of A or B is a cEt nucleoside, another of A or B comprises a 2′-F sugar moiety, and W comprises a 5′-(R)-Me DNA sugar moiety. In certain embodiments, one of A or B is an α-L-LNA nucleoside, another of A or B comprises a 2′-F sugar moiety, and W comprises a 5′-(R)-Me DNA sugar moiety.

In certain embodiments, one of A or B comprises a bicyclic sugar moiety, another of A or B comprises a 2′-(ara)-F sugar moiety, and W comprises a 5′-(R)-Me DNA sugar moiety. In certain embodiments, one of A or B is an LNA nucleoside, another of A or B comprises a 2′-(ara)-F sugar moiety, and W comprises a 5′-(R)-Me DNA sugar moiety. In certain embodiments, one of A or B is a cEt nucleoside, another of A or B comprises a 2′-(ara)-F sugar moiety, and W comprises a 5′-(R)-Me DNA sugar moiety. In certain embodiments, one of A or B is an α-L-LNA nucleoside, another of A or B comprises a 2′-(ara)-F sugar moiety, and W comprises a 5′-(R)-Me DNA sugar moiety.

In certain embodiments, at least two of A, B or W comprises a 2′-substituted sugar moiety, and the other comprises a bicyclic sugar moiety. In certain embodiments, at least two of A, B or W comprises a bicyclic sugar moiety, and the other comprises a 2′-substituted sugar moiety. In certain embodiments, a gapmer has a sugar motif other than: E-K-K-(D)₉-K-K-E; E-E-E-E-K-(D)₉-E-E-E-E-E; E-K-K-K-(D)₉-K-K-K-E; K-E-E-K-(D)₉-K-E-E-K; K-D-D-K-(D)₉-K-D-D-K; K-E-K-E-K-(D)₉-K-E-K-E-K; K-D-K-D-K-(D)₉-K-D-K-D-K; E-K-E-K-(D)₉-K-E-K-E; E-E-E-E-E-K-(D)₈-E-E-E-E-E; or E-K-E-K-E-(D)₉-E-K-E-K-E, E-E-E-K-K-(D)₇-E-E-K, E-K-E-K-K-K-(D)₇-K-E-K-E, E-K-E-K-E-K-(D)₇-K-E-K-E, wherein K is a nucleoside comprising a cEt sugar moiety and E is a nucleoside comprising a 2′-MOE sugar moiety.

In certain embodiments a gapmer comprises a A-(D)₄-A-(D)₄-A-(D)₄-AA motif. In certain embodiments a gapmer comprises a B-(D)₄-A-(D)₄-A-(D)₄-AA motif. In certain embodiments a gapmer comprises a A-(D)₄-B-(D)₄-A-(D)₄-AA motif. In certain embodiments a gapmer comprises a A-(D)₄-A-(D)₄-B-(D)₄-AA motif. In certain embodiments a gapmer comprises a A-(D)₄-A-(D)₄-A-(D)₄-BA motif. In certain embodiments a gapmer comprises a A-(D)₄-A-(D)₄-A-(D)₄-BB motif. In certain embodiments a gapmer comprises a K-(D)₄-K-(D)₄-K-(D)₄-K-E motif

viii. Certain Internucleoside Linkage Motifs

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif, as described above for nucleoside motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The nucleoside motif is independently selected, so such oligonucleotides having a gapped internucleoside linkage motif may or may not have a gapped nucleoside motif and if it does have a gapped nucleoside motif, the wing and gap lengths may or may not be the same.

In certain embodiments, oligonucleotides comprise a region having an alternating internucleoside linkage motif. In certain embodiments, oligonucleotides of the present invention comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosphonate linkages. In certain embodiments, oligonucleotides having a gapmer nucleoside motif comprise a linkage motif comprising all phosphorothioate linkages except for one or two methylphosphonate linkages. In certain embodiments, one methylphosphonate linkage is in the central gap of an oligonucleotide having a gapmer nucleoside motif

ix. Certain Modification Motifs

Modification motifs define oligonucleotides by nucleoside motif (sugar motif and nucleobase motif) and linkage motif. For example, certain oligonucleotides have the following modification motif:
AₛAₛAₛDₛDₛDₛDₛ(^{N}D)ₛDₛDₛDₛDₛBₛBₛB;
   wherein each A is a modified nucleoside comprising a 2′-substituted sugar moiety; each D is an unmodified 2′-deoxynucleoside; each B is a modified nucleoside comprising a bicyclic sugar moiety; ^{N}D is a modified nucleoside comprising a modified nucleobase; and s is a phosphorothioate internucleoside linkage. Thus, the sugar motif is a gapmer motif. The nucleobase modification motif is a single modified nucleobase at 8^{th} nucleoside from the 5′-end. Combining the sugar motif and the nucleobase modification motif, the nucleoside motif is an interrupted gapmer where the gap of the sugar modified gapmer is interrupted by a nucleoside comprising a modified nucleobase. The linkage motif is uniform phosphorothioate. The following non-limiting Table further illustrates certain modification motifs:

TABLE 13
Certain Modification Motifs

| 5′-wing | Central gap | 3′-wing |
|---|---|---|
| region | region | region |
| BₛBₛ | ₛDₛDₛDₛDₛDₛDₛDₛDₛDₛ | AₛAₛAₛAₛAₛAₛAₛA |
| AsBsBs | DsDsDsDsDsDsDsDsDs | BsBsA |
| AsBsBs | DsDsDsDs(^{N}D)sDsDsDsDs | BsBsA |
| AsBsBs | DsDsDsDsAsDsDsDsDs | BsBsA |
| AsBsBs | DsDsDsDsBsDsDsDsDs | BsBsA |
| AsBsBs | DsDsDsDsWsDsDsDsDs | BsBsA |
| AsBsBsBs | DsDsDsDsDsDsDsDsDs | BsBsAsBsB |
| AsBsBs | DsDsDsDsDsDsDsDsDs | BsBsAsBsB |
| BsBsAsBsBs | DsDsDsDsDsDsDsDsDs | BsBsA |
| AsBsBs | DsDsDsDsDsDsDsDsDs | BsBsAsBsBsBsB |
| AsAsBsAsAs | DsDsDsDsDsDsDsDsDs | BsBsA |
| AsAsAsBsAsAs | DsDsDsDsDsDsDsDsDs | BsBsA |
| AsAsBsAsAs | DsDsDsDsDsDsDsDsDs | AsAsBsAsA |
| AsAsAsBsAsAs | DsDsDsDsDsDsDsDsDs | AsAsBsAsAsA |
| AsAsAsAsBsAsAs | DsDsDsDsDsDsDsDsDs | BsBsA |
| AsBsAsBs | DsDsDsDsDsDsDsDsDs | BsAsBsA |
| AsBsAsBs | DsDsDsDsDsDsDsDsDs | AsAsBsAsAs |
| AsBsBs | DsDsDsDsDsDsDsDsDs | BsAsBsA |
| BsBsAsBsBsBsB | DsDsDsDsDsDsDsDsDs | BsAsBsA |
| AsAsAsAsAs | DsDsDsDsDsDsDsDsDs | AsAsAsAsA |
| AsAsAsAsAs | DsDsDsDsDsDsDs | AsAsAsAsA |
| AsAsAsAsAs | DsDsDsDsDsDsDsDsDs | BsBsAsBsBsBsB |
| AsAsAsBsBs | DsDsDsDsDsDsDs | BsBsA |
| AsBsAsBs | DsDsDsDsDsDsDsDs | BsBsA |
| AsBsAsBs | DsDsDsDsDsDsDs | AsAsAsBsBs |
| AsAsAsAsBs | DsDsDsDsDsDsDs | BsAsAsAsA |
| BsBs | DsDsDsDsDsDsDsDs | AsA |
| AsAs | DsDsDsDsDsDsDs | AsAsAsAsAsAsAsA |
| AsAsAs | DsDsDsDsDsDsDs | AsAsAsAsAsAsA |
| AsAsAs | DsDsDsDsDsDsDs | AsAsAsAsAsA |
| AsBs | DsDsDsDsDsDsDs | BsBsBsA |
| AsBsBsBs | DsDsDsDsDsDsDsDsDs | BsA |
| AsBs | DsDsDsDsDsDsDsDsDs | BsBsBsA |
| AsAsAsBsBs | DsDsDs(^{N}D)sDsDsDs | BsBsAsAsA |
| AsAsAsBsBs | DsDsDsAsDsDsDs | BsBsAsAsA |
| AsAsAsBsBs | DsDsDsBsDsDsDs | BsBsAsAsA |
| AsAsAsAsBs | DsDsDsDsDsDsDs | BsAsAsAsA |
| AsAsBsBsBs | DsDsDsDsDsDsDs | BsBsBsAsA |
| AsAsAsAsBs | DsDsDsDsDsDsDs | AsAsAsAsAs |
| AsAsAsBsBs | DsDsDsDsDsDsDs | AsAsAsAsAs |
| AsAsBsBsBs | DsDsDsDsDsDsDs | AsAsAsAsAs |
| AsAsAsAsAs | DsDsDsDsDsDsDs | BsAsAsAsAs |
| AsAsAsAsAs | DsDsDsDsDsDsDs | BsBsAsAsAs |
| AsAsAsAsAs | DsDsDsDsDsDsDs | BsBsBsAsAs |
| AsBsBs | DsDsDsDs(^{N}D)s(^{N}D)sDsDsDs | BsBsA |
| AsBsBs | Ds(^{N}D)s(^{N}D)sDs(^{N}D)s(^{N}D)sDs(^{N}D)s(^{N}D)s | BsBsA |
| AsBsBs | Ds(^{N}D)sDsDsDsDsDsDsDs | BsBsA |
| AsBsBs | DsDs(^{N}D)sDsDsDsDsDsDs | BsBsA |
| AsBsBs | Ds(^{N}D)s(^{N}D)sDsDsDsDsDsDs | BsBsA |
| AsBsBs | DsDs(D)zDsDsDsDsDsDs | BsBsA |
| AsBsBs | Ds(D)zDsDsDsDsDsDsDs | BsBsA |
| AsBsBs | (D)zDsDsDsDsDsDsDsDs | BsBsA |
| AsBsBs | DsDsAsDsDsDsDsDsDs | BsBsA |
| AsBsBs | DsDsBsDsDsDsDsDsDs | BsBsA |
| AsBsBs | AsDsDsDsDsDsDsDsDs | BsBsA |
| AsBsBs | BsDsDsDsDsDsDsDsDs | BsBsA |
| AsBsAsBs | DsDs(D)zDsDsDsDsDsDs | BsBsBsAsAs |
| AsAsAsBsBs | DsDs(^{N}D)sDsDsDsDsDsDs | AsA |
| AsBsBsBs | Ds(D)zDsDsDsDsDsDsDs | AsAsAsBsBs |
| AsBsBs | DsDsDsDsDsDsDsDs(D)z | BsBsA |
| AsAsBsBsBs | DsDsDsAsDsDsDs | BsBsBsAsA |
| AsAsBsBsBs | DsDsDsBsDsDsDs | BsBsBsAsA |
| AsBsAsBs | DsDsDsAsDsDsDs | BsBsAsBsBsBsB |
| AsBsBsBs | DsDsDsDs(D)zDsDsDsDs | BsA |
| AsAsBsBsBs | DsDsAsAsDsDsDs | BsBsA |
| AsBsBs | DsDsDsDs(D)zDsDsDsDs | BsBsBsA |
| BsBs | DsDs(^{N}D)sDs(^{N}D)sDsDsDsDs | BsBsAsBsBsBsB |

wherein each A and B are nucleosides comprising differently modified sugar moieties, each D is a nucleoside comprising an unmodified 2′deoxy sugar moiety, each W is a modified nucleoside of either the first type, the second type or a third type, each ^{N}D is a modified nucleoside comprising a modified nucleobase, s is a phosphorothioate internucleoside linkage, and z is a non-phosphorothioate internucleoside linkage.

In certain embodiments, each A comprises a modified sugar moiety. In certain embodiments, each A comprises a 2′-substituted sugar moiety. In certain embodiments, each A comprises a 2′-substituted sugar moiety selected from among F, (ara)-F, OCH₃ and O(CH₂)₂—OCH₃. In certain embodiments, each A comprises a bicyclic sugar moiety. In certain embodiments, each A comprises a bicyclic sugar moiety selected from among cEt, cMOE, LNA, α-L-LNA, ENA and 2′-thio LNA. In certain embodiments, each A comprises a modified nucleobase. In certain embodiments, each A comprises a modified nucleobase selected from among 2-thio-thymidine nucleoside and 5-propyne uridine nucleoside. In certain embodiments, each B comprises a modified sugar moiety. In certain embodiments, each B comprises a 2′-substituted sugar moiety. In certain embodiments, each B comprises a 2′-substituted sugar moiety selected from among F, (ara)-F, OCH₃ and O(CH₂)₂—OCH₃. In certain embodiments, each B comprises a bicyclic sugar moiety. In certain embodiments, each B comprises a bicyclic sugar moiety selected from among cEt, cMOE, LNA, α-L-LNA, ENA and 2′-thio LNA. In certain embodiments, each B comprises a modified nucleobase. In certain embodiments, each B comprises a modified nucleobase selected from among 2-thio-thymidine nucleoside and 5-propyne uridine nucleoside. In certain embodiments, each A comprises an HNA. In certain embodiments, each A comprises an F-HNA.

In certain embodiments, each W comprises a modified sugar moiety. In certain embodiments, each W comprises a 2′-substituted sugar moiety. In certain embodiments, each W comprises a 2′-substituted sugar moiety selected from among F, (ara)-F, OCH₃ and O(CH₂)₂—OCH₃. In certain embodiments, each W comprises a 5′-substituted sugar moiety. In certain embodiments, each W comprises a 5′-substituted sugar moiety selected from among 5′-Me, and 5′-(R)-Me. In certain embodiments, each W comprises a bicyclic sugar moiety. In certain embodiments, each W comprises a bicyclic sugar moiety selected from among cEt, cMOE, LNA, α-L-LNA, ENA and 2′-thio LNA. In certain embodiments, each W comprises a sugar surrogate. In certain embodiments, each W comprises a sugar surrogate selected from among HNA and F-HNA.

In certain embodiments, at least one of A or B comprises a bicyclic sugar moiety, and the other comprises a 2′-substituted sugar moiety. In certain embodiments, one of A or B is an LNA nucleoside and the other of A or B comprises a 2′-substituted sugar moiety. In certain embodiments, one of A or B is a cEt nucleoside and the other of A or B comprises a 2′-substituted sugar moiety. In certain embodiments, one of A or B is an α-L-LNA nucleoside and the other of A or B comprises a 2′-substituted sugar moiety. In certain embodiments, one of A or B is an LNA nucleoside and the other of A or B comprises a 2′-MOE sugar moiety. In certain embodiments, one of A or B is a cEt nucleoside and the other of A or B comprises a 2′-MOE sugar moiety. In certain embodiments, one of A or B is an α-L-LNA nucleoside and the other of A or B comprises a 2′-MOE sugar moiety. In certain embodiments, one of A or B is an LNA nucleoside and the other of A or B comprises a 2′-F sugar moiety. In certain embodiments, one of A or B is a cEt nucleoside and the other of A or B comprises a 2′-F sugar moiety. In certain embodiments, one of A or B is an α-L-LNA nucleoside and the other of A or B comprises a 2′-F sugar moiety. In certain embodiments, one of A or B is an LNA nucleoside and the other of A or B comprises a 2′-(ara)-F sugar moiety. In certain embodiments, one of A or B is a cEt nucleoside and the other of A or B comprises a 2′-(ara)-F sugar moiety. In certain embodiments, one of A or B is an α-L-LNA nucleoside and the other of A or B comprises a 2′-(ara)-F sugar moiety.

In certain embodiments, A comprises a bicyclic sugar moiety, and B comprises a 2′-substituted sugar moiety. In certain embodiments, A is an LNA nucleoside and B comprises a 2′-substituted sugar moiety. In certain embodiments, A is a cEt nucleoside and B comprises a 2′-substituted sugar moiety. In certain embodiments, A is an α-L-LNA nucleoside and B comprises a 2′-substituted sugar moiety.

In certain embodiments, A comprises a bicyclic sugar moiety, and B comprises a 2′-MOE sugar moiety. In certain embodiments, A is an LNA nucleoside and B comprises a 2′-MOE sugar moiety. In certain embodiments, A is a cEt nucleoside and B comprises a 2′-MOE sugar moiety. In certain embodiments, A is an α-L-LNA nucleoside and B comprises a 2′-MOE sugar moiety.

In certain embodiments, A comprises a bicyclic sugar moiety, and B comprises a 2′-F sugar moiety. In certain embodiments, A is an LNA nucleoside and B comprises a 2′-F sugar moiety. In certain embodiments, A is a cEt nucleoside and B comprises a 2′-F sugar moiety. In certain embodiments, A is an α-L-LNA nucleoside and B comprises a 2′-F sugar moiety.

In certain embodiments, A comprises a bicyclic sugar moiety, and B comprises a 2′-(ara)-F sugar moiety. In certain embodiments, A is an LNA nucleoside and B comprises a 2′-(ara)-F sugar moiety. In certain embodiments, A is a cEt nucleoside and B comprises a 2′-(ara)-F sugar moiety. In certain embodiments, A is an α-L-LNA nucleoside and B comprises a 2′-(ara)-F sugar moiety.

In certain embodiments, B comprises a bicyclic sugar moiety, and A comprises a 2′-MOE sugar moiety. In certain embodiments, B is an LNA nucleoside and A comprises a 2′-MOE sugar moiety. In certain embodiments, B is a cEt nucleoside and A comprises a 2′-MOE sugar moiety. In certain embodiments, B is an α-L-LNA nucleoside and A comprises a 2′-MOE sugar moiety.

In certain embodiments, B comprises a bicyclic sugar moiety, and A comprises a 2′-F sugar moiety. In certain embodiments, B is an LNA nucleoside and A comprises a 2′-F sugar moiety. In certain embodiments, B is a cEt nucleoside and A comprises a 2′-F sugar moiety. In certain embodiments, B is an α-L-LNA nucleoside and A comprises a 2′-F sugar moiety.

In certain embodiments, B comprises a bicyclic sugar moiety, and A comprises a 2′-(ara)-F sugar moiety. In certain embodiments, B is an LNA nucleoside and A comprises a 2′-(ara)-F sugar moiety. In certain embodiments, B is a cEt nucleoside and A comprises a 2′-(ara)-F sugar moiety. In certain embodiments, B is an α-L-LNA nucleoside and A comprises a 2′-(ara)-F sugar moiety.

In certain embodiments, at least one of A or B comprises a bicyclic sugar moiety, another of A or B comprises a 2′-substituted sugar moiety and W comprises a modified nucleobase. In certain embodiments, one of A or B is an LNA nucleoside, another of A or B comprises a 2′-substituted sugar moiety, and W comprises a modified nucleobase. In certain embodiments, one of A or B is a cEt nucleoside, another of A or B comprises a 2′-substituted sugar moiety, and C comprises a modified nucleobase. In certain embodiments, one of A or B is an α-L-LNA nucleoside, another of A or B comprises a 2′-substituted sugar moiety, and W comprises a modified nucleobase.

In certain embodiments, one of A or B comprises a bicyclic sugar moiety, another of A or B comprises a 2′-MOE sugar moiety, and W comprises a modified nucleobase. In certain embodiments, one of A or B is an LNA nucleoside, another of A or B comprises a 2′-MOE sugar moiety, and W comprises a modified nucleobase. In certain embodiments, one of A or B is a cEt nucleoside, another of A or B comprises a 2′-MOE sugar moiety, and W comprises a modified nucleobase. In certain embodiments, one of A or B is an α-L-LNA nucleoside, another of A or B comprises a 2′-MOE sugar moiety, and W comprises a modified nucleobase.

In certain embodiments, one of A or B comprises a bicyclic sugar moiety, another of A or B comprises a 2′-F sugar moiety, and W comprises a modified nucleobase. In certain embodiments, one of A or B is an LNA nucleoside, another of A or B comprises a 2′-F sugar moiety, and W comprises a modified nucleobase. In certain embodiments, one of A or B is a cEt nucleoside, another of A or B comprises a 2′-F sugar moiety, and W comprises a modified nucleobase. In certain embodiments, one of A or B is an α-L-LNA nucleoside, another of A or B comprises a 2′-F sugar moiety, and W comprises a modified nucleobase.

In certain embodiments, one of A or B comprises a bicyclic sugar moiety, another of A or B comprises a 2′-(ara)-F sugar moiety, and W comprises a modified nucleobase. In certain embodiments, one of A or B is an LNA nucleoside, another of A or B comprises a 2′-(ara)-F sugar moiety, and W comprises a modified nucleobase. In certain embodiments, one of A or B is a cEt nucleoside, another of A or B comprises a 2′-(ara)-F sugar moiety, and W comprises a modified nucleobase. In certain embodiments, one of A or B is an α-L-LNA nucleoside, another of A or B comprises a 2′-(ara)-F sugar moiety, and W comprises a modified nucleobase.

In certain embodiments, one of A or B comprises a bicyclic sugar moiety, another of A or B comprises a 2′-substituted sugar moiety, and W comprises a 2-thio-thymidine nucleobase. In certain embodiments, one of A or B is an LNA nucleoside, another of A or B comprises a 2′-substituted sugar moiety, and W comprises a 2-thio-thymidine nucleobase. In certain embodiments, one of A or B is a cEt nucleoside, another of A or B comprises a 2′-substituted sugar moiety, and W comprises a 2-thio-thymidine nucleobase. In certain embodiments, one of A or B is an α-L-LNA nucleoside, another of A or B comprises a 2′-substituted sugar moiety, and W comprises a 2-thio-thymidine nucleobase.

In certain embodiments, one of A or B comprises a bicyclic sugar moiety, another of A or B comprises a 2′-MOE sugar moiety, and W comprises a 2-thio-thymidine nucleobase. In certain embodiments, one of A or B is an LNA nucleoside, another of A or B comprises a 2′-MOE sugar moiety, and W comprises a 2-thio-thymidine nucleobase. In certain embodiments, one of A or B is a cEt nucleoside, another of A or B comprises a 2′-MOE sugar moiety, and W comprises a 2-thio-thymidine nucleobase. In certain embodiments, one of A or B is an α-L-LNA nucleoside, another of A or B comprises a 2′-MOE sugar moiety, and W comprises a 2-thio-thymidine nucleobase.

In certain embodiments, one of A or B comprises a bicyclic sugar moiety, another of A or B comprises a 2′-F sugar moiety, and W comprises a 2-thio-thymidine nucleobase. In certain embodiments, one of A or B is an LNA nucleoside, another of A or B comprises a 2′-F sugar moiety, and W comprises a 2-thio-thymidine nucleobase. In certain embodiments, one of A or B is a cEt nucleoside, another of A or B comprises a 2′-F sugar moiety, and W comprises a 2-thio-thymidine nucleobase. In certain embodiments, one of A or B is an α-L-LNA nucleoside, another of A or B comprises a 2′-F sugar moiety, and W comprises a 2-thio-thymidine nucleobase.

In certain embodiments, one of A or B comprises a bicyclic sugar moiety, another of A or B comprises a 2′-(ara)-F sugar moiety, and W comprises a 2-thio-thymidine nucleobase. In certain embodiments, one of A or B is an LNA nucleoside, another of A or B comprises a 2′-(ara)-F sugar moiety, and W comprises a 2-thio-thymidine nucleobase. In certain embodiments, one of A or B is a cEt nucleoside, another of A or B comprises a 2′-(ara)-F sugar moiety, and W comprises a 2-thio-thymidine nucleobase. In certain embodiments, one of A or B is an α-L-LNA nucleoside, another of A or B comprises a 2′-(ara)-F sugar moiety, and W comprises 2-thio-thymidine nucleobase.

In certain embodiments, one of A or B comprises a bicyclic sugar moiety, another of A or B comprises a 2′-MOE sugar moiety, and W comprises a 5-propyne uridine nucleobase. In certain embodiments, one of A or B is an LNA nucleoside, another of A or B comprises a 2′-MOE sugar moiety, and C comprises a 5-propyne uridine nucleobase. In certain embodiments, one of A or B is a cEt nucleoside, another of A or B comprises a 2′-MOE sugar moiety, and W comprises a 5-propyne uridine nucleobase. In certain embodiments, one of A or B is an α-L-LNA nucleoside, another of A or B comprises a 2′-MOE sugar moiety, and C comprises a 5-propyne uridine nucleobase.

In certain embodiments, one of A or B comprises a bicyclic sugar moiety, another of A or B comprises a 2′-F sugar moiety, and W comprises a 5-propyne uridine nucleobase. In certain embodiments, one of A or B is an LNA nucleoside, another of A or B comprises a 2′-F sugar moiety, and C comprises a 5-propyne uridine nucleobase. In certain embodiments, one of A or B is a cEt nucleoside, another of A or B comprises a 2′-F sugar moiety, and W comprises a 5-propyne uridine nucleobase. In certain embodiments, one of A or B is an α-L-LNA nucleoside, another of A or B comprises a 2′-F sugar moiety, and W comprises a 5-propyne uridine nucleobase.

In certain embodiments, one of A or B comprises a bicyclic sugar moiety, another of A or B comprises a 2′-(ara)-F sugar moiety, and W comprises a 5-propyne uridine nucleobase. In certain embodiments, one of A or B is an LNA nucleoside, another of A or B comprises a 2′-(ara)-F sugar moiety, and W comprises a 5-propyne uridine nucleobase. In certain embodiments, one of A or B is a cEt nucleoside, another of A or B comprises a 2′-(ara)-F sugar moiety, and W comprises a 5-propyne uridine nucleobase. In certain embodiments, one of A or B is an α-L-LNA nucleoside, another of A or B comprises a 2′-(ara)-F sugar moiety, and W comprises a 5-propyne uridine nucleobase.

In certain embodiments, one of A or B comprises a bicyclic sugar moiety, another of A or B comprises a 2′-MOE sugar moiety, and W comprises a sugar surrogate. In certain embodiments, one of A or B is an LNA nucleoside, another of A or B comprises a 2′-MOE sugar moiety, and W comprises a sugar surrogate. In certain embodiments, one of A or B is a cEt nucleoside, another of A or B comprises a 2′-MOE sugar moiety, and W comprises a sugar surrogate. In certain embodiments, one of A or B is an α-L-LNA nucleoside, another of A or B comprises a 2′-MOE sugar moiety, and W comprises a sugar surrogate.

In certain embodiments, one of A or B comprises a bicyclic sugar moiety, another of A or B comprises a 2′-F sugar moiety, and W comprises a sugar surrogate. In certain embodiments, one of A or B is an LNA nucleoside, another of A or B comprises a 2′-F sugar moiety, and W comprises a sugar surrogate. In certain embodiments, one of A or B is a cEt nucleoside, another of A or B comprises a 2′-F sugar moiety, and W comprises a sugar surrogate. In certain embodiments, one of A or B is an α-L-LNA nucleoside, another of A or B comprises a 2′-F sugar moiety, and W comprises a sugar surrogate.

In certain embodiments, one of A or B comprises a bicyclic sugar moiety, another of A or B comprises a 2′-(ara)-F sugar moiety, and W comprises a sugar surrogate. In certain embodiments, one of A or B is an LNA nucleoside, another of A or B comprises a 2′-(ara)-F sugar moiety, and W comprises a sugar surrogate. In certain embodiments, one of A or B is a cEt nucleoside, another of A or B comprises a 2′-(ara)-F sugar moiety, and W comprises a sugar surrogate. In certain embodiments, one of A or B is an α-L-LNA nucleoside, another of A or B comprises a 2′-(ara)-F sugar moiety, and W comprises sugar surrogate.

In certain embodiments, one of A or B comprises a bicyclic sugar moiety, another of A or B comprises a 2′-MOE sugar moiety, and W comprises a HNA sugar surrogate. In certain embodiments, one of A or B is an LNA nucleoside, another of A or B comprises a 2′-MOE sugar moiety, and W comprises a HNA sugar surrogate. In certain embodiments, one of A or B is a cEt nucleoside, another of A or B comprises a 2′-MOE sugar moiety, and W comprises a HNA sugar surrogate. In certain embodiments, one of A or B is an α-L-LNA nucleoside, another of A or B comprises a 2′-MOE sugar moiety, and W comprises a HNA sugar surrogate.

In certain embodiments, one of A or B comprises a bicyclic sugar moiety, another of A or B comprises a 2′-F sugar moiety, and W comprises a HNA sugar surrogate. In certain embodiments, one of A or B is an LNA nucleoside, another of A or B comprises a 2′-F sugar moiety, and W comprises a HNA sugar surrogate. In certain embodiments, one of A or B is a cEt nucleoside, another of A or B comprises a 2′-F sugar moiety, and W comprises a HNA sugar surrogate. In certain embodiments, one of A or B is an α-L-LNA nucleoside, another of A or B comprises a 2′-F sugar moiety, and W comprises a sugar HNA surrogate.

In certain embodiments, one of A or B comprises a bicyclic sugar moiety, another of A or B comprises a 2′-(ara)-F sugar moiety, and W comprises a HNA sugar surrogate. In certain embodiments, one of A or B is an LNA nucleoside, another of A or B comprises a 2′-(ara)-F sugar moiety, and W comprises a HNA sugar surrogate. In certain embodiments, one of A or B is a cEt nucleoside, another of A or B comprises a 2′-(ara)-F sugar moiety, and W comprises a HNA sugar surrogate. In certain embodiments, one of A or B is an α-L-LNA nucleoside, another of A or B comprises a 2′-(ara)-F sugar moiety, and W comprises a HNA sugar surrogate.

In certain embodiments, one of A or B comprises a bicyclic sugar moiety, another of A or B comprises a 2′-MOE sugar moiety, and W comprises a F-HNA sugar surrogate. In certain embodiments, one of A or B is an LNA nucleoside, another of A or B comprises a 2′-MOE sugar moiety, and W comprises a F-HNA sugar surrogate. In certain embodiments, one of A or B is a cEt nucleoside, another of A or B comprises a 2′-MOE sugar moiety, and W comprises a F-HNA sugar surrogate. In certain embodiments, one of A or B is an α-L-LNA nucleoside, another of A or B comprises a 2′-MOE sugar moiety, and W comprises a F-HNA sugar surrogate.

In certain embodiments, one of A or B comprises a bicyclic sugar moiety, another of A or B comprises a 2′-F sugar moiety, and W comprises a F-HNA sugar surrogate. In certain embodiments, one of A or B is an LNA nucleoside, another of A or B comprises a 2′-F sugar moiety, and W comprises a F-HNA sugar surrogate. In certain embodiments, one of A or B is a cEt nucleoside, another of A or B comprises a 2′-F sugar moiety, and W comprises a F-HNA sugar surrogate. In certain embodiments, one of A or B is an α-L-LNA nucleoside, another of A or B comprises a 2′-F sugar moiety, and W comprises a F-HNA sugar surrogate.

In certain embodiments, one of A or B comprises a bicyclic sugar moiety, another of A or B comprises a 2′-(ara)-F sugar moiety, and W comprises a F-HNA sugar surrogate. In certain embodiments, one of A or B is an LNA nucleoside, another of A or B comprises a 2′-(ara)-F sugar moiety, and W comprises a F-HNA sugar surrogate. In certain embodiments, one of A or B is a cEt nucleoside, another of A or B comprises a 2′-(ara)-F sugar moiety, and W comprises a F-HNA sugar surrogate. In certain embodiments, one of A or B is an α-L-LNA nucleoside, another of A or B comprises a 2′-(ara)-F sugar moiety, and W comprises a F-HNA sugar surrogate.

In certain embodiments, one of A or B comprises a bicyclic sugar moiety, another of A or B comprises a 2′-MOE sugar moiety, and W comprises a 5′-Me DNA sugar moiety. In certain embodiments, one of A or B is an LNA nucleoside, another of A or B comprises a 2′-MOE sugar moiety, and W comprises a 5′-Me DNA sugar moiety. In certain embodiments, one of A or B is a cEt nucleoside, another of A or B comprises a 2′-MOE sugar moiety, and W comprises a 5′-Me DNA sugar moiety. In certain embodiments, one of A or B is an α-L-LNA nucleoside, another of A or B comprises a 2′-MOE sugar moiety, and W comprises a 5′-Me DNA sugar moiety.

In certain embodiments, one of A or B comprises a bicyclic sugar moiety, another of A or B comprises a 2′-F sugar moiety, and W comprises a 5′-Me DNA sugar moiety. In certain embodiments, one of A or B is an LNA nucleoside, another of A or B comprises a 2′-F sugar moiety, and W comprises a 5′-Me DNA sugar moiety. In certain embodiments, one of A or B is a cEt nucleoside, another of A or B comprises a 2′-F sugar moiety, and W comprises a 5′-Me DNA sugar moiety. In certain embodiments, one of A or B is an α-L-LNA nucleoside, another of A or B comprises a 2′-F sugar moiety, and W comprises a 5′-Me DNA sugar moiety.

In certain embodiments, one of A or B comprises a bicyclic sugar moiety, another of A or B comprises a 2′-(ara)-F sugar moiety, and W comprises a 5′-Me DNA sugar moiety. In certain embodiments, one of A or B is an LNA nucleoside, another of A or B comprises a 2′-(ara)-F sugar moiety, and W comprises a 5′-Me DNA sugar moiety. In certain embodiments, one of A or B is a cEt nucleoside, another of A or B comprises a 2′-(ara)-F sugar moiety, and W comprises a 5′-Me DNA sugar moiety. In certain embodiments, one of A or B is an α-L-LNA nucleoside, another of A or B comprises a 2′-(ara)-F sugar moiety, and W comprises a 5′-Me DNA sugar moiety.

In certain embodiments, one of A or B comprises a bicyclic sugar moiety, another of A or B comprises a 2′-MOE sugar moiety, and W comprises a 5′-(R)-Me DNA sugar moiety. In certain embodiments, one of A or B is an LNA nucleoside, another of A or B comprises a 2′-MOE sugar moiety, and W comprises a 5′-(R)-Me DNA sugar moiety. In certain embodiments, one of A or B is a cEt nucleoside, another of A or B comprises a 2′-MOE sugar moiety, and W comprises a 5′-(R)-Me DNA sugar moiety. In certain embodiments, one of A or B is an α-L-LNA nucleoside, another of A or B comprises a 2′-MOE sugar moiety, and W comprises a 5′-(R)-Me DNA sugar moiety.

In certain embodiments, one of A or B comprises a bicyclic sugar moiety, another of A or B comprises a 2′-F sugar moiety, and W comprises a 5′-(R)-Me DNA sugar moiety. In certain embodiments, one of A or B is an LNA nucleoside, another of A or B comprises a 2′-F sugar moiety, and W comprises a 5′-(R)-Me DNA sugar moiety. In certain embodiments, one of A or B is a cEt nucleoside, another of A or B comprises a 2′-F sugar moiety, and W comprises a 5′-(R)-Me DNA sugar moiety. In certain embodiments, one of A or B is an α-L-LNA nucleoside, another of A or B comprises a 2′-F sugar moiety, and W comprises a 5′-(R)-Me DNA sugar moiety.

In certain embodiments, one of A or B comprises a bicyclic sugar moiety, another of A or B comprises a 2′-(ara)-F sugar moiety, and W comprises a 5′-(R)-Me DNA sugar moiety. In certain embodiments, one of A or B is an LNA nucleoside, another of A or B comprises a 2′-(ara)-F sugar moiety, and W comprises a 5′-(R)-Me DNA sugar moiety. In certain embodiments, one of A or B is a cEt nucleoside, another of A or B comprises a 2′-(ara)-F sugar moiety, and W comprises a 5′-(R)-Me DNA sugar moiety. In certain embodiments, one of A or B is an α-L-LNA nucleoside, another of A or B comprises a 2′-(ara)-F sugar moiety, and W comprises a 5′-(R)-Me DNA sugar moiety.

In certain embodiments, at least two of A, B or W comprises a 2′-substituted sugar moiety, and the other comprises a bicyclic sugar moiety. In certain embodiments, at least two of A, B or W comprises a bicyclic sugar moiety, and the other comprises a 2′-substituted sugar moiety.

d. Certain Overall Lengths

In certain embodiments, the present invention provides oligomeric compounds including oligonucleotides of any of a variety of ranges of lengths. In certain embodiments, the invention provides oligomeric compounds or oligonucleotides consisting of X to Y linked nucleosides, where X represents the fewest number of nucleosides in the range and Y represents the largest number of nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, the invention provides oligomeric compounds which comprise oligonucleotides consisting of 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 9 to 26, 9 to 27, 9 to 28, 9 to 29, 9 to 30, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 11 to 26, 11 to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides. In embodiments where the number of nucleosides of an oligomeric compound or oligonucleotide is limited, whether to a range or to a specific number, the oligomeric compound or oligonucleotide may, nonetheless further comprise additional other substituents. For example, an oligonucleotide comprising 8-30 nucleosides excludes oligonucleotides having 31 nucleosides, but, unless otherwise indicated, such an oligonucleotide may further comprise, for example one or more conjugates, terminal groups, or other substituents. In certain embodiments, a gapmer oligonucleotide has any of the above lengths.

Further, where an oligonucleotide is described by an overall length range and by regions having specified lengths, and where the sum of specified lengths of the regions is less than the upper limit of the overall length range, the oligonucleotide may have additional nucleosides, beyond those of the specified regions, provided that the total number of nucleosides does not exceed the upper limit of the overall length range.

e. Certain Oligonucleotides

In certain embodiments, oligonucleotides of the present invention are characterized by their modification motif and overall length. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar-gapmer may be the same or different from one another and may be the same or different from the internucleoside linkages of the gap region. Likewise, such sugar-gapmer oligonucleotides may comprise one or more modified nucleobase independent of the gapmer pattern of the sugar modifications. One of skill in the art will appreciate that such motifs may be combined to create a variety of oligonucleotides. Herein if a description of an oligonucleotide or oligomeric compound is silent with respect to one or more parameter, such parameter is not limited. Thus, an oligomeric compound described only as having a gapmer sugar motif without further description may have any length, internucleoside linkage motif, and nucleobase modification motif Unless otherwise indicated, all chemical modifications are independent of nucleobase sequence.

f. Certain Conjugate Groups

In certain embodiments, oligomeric compounds are modified by attachment of one or more conjugate groups. In general, conjugate groups modify one or more properties of the attached oligomeric compound including but not limited to pharmacodynamics, pharmacokinetics, stability, binding, absorption, cellular distribution, cellular uptake, charge and clearance. Conjugate groups are routinely used in the chemical arts and are linked directly or via an optional conjugate linking moiety or conjugate linking group to a parent compound such as an oligomeric compound, such as an oligonucleotide. Conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

In certain embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

In certain embodiments, conjugate groups are directly attached to oligonucleotides in oligomeric compounds. In certain embodiments, conjugate groups are attached to oligonucleotides by a conjugate linking group. In certain such embodiments, conjugate linking groups, including, but not limited to, bifunctional linking moieties such as those known in the art are amenable to the compounds provided herein. Conjugate linking groups are useful for attachment of conjugate groups, such as chemical stabilizing groups, functional groups, reporter groups and other groups to selective sites in a parent compound such as for example an oligomeric compound. In general a bifunctional linking moiety comprises a hydrocarbyl moiety having two functional groups. One of the functional groups is selected to bind to a parent molecule or compound of interest and the other is selected to bind essentially any selected group such as chemical functional group or a conjugate group. In some embodiments, the conjugate linker comprises a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Some nonlimiting examples of conjugate linking moieties include pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other linking groups include, but are not limited to, substituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

Conjugate groups may be attached to either or both ends of an oligonucleotide (terminal conjugate groups) and/or at any internal position.

In certain embodiments, conjugate groups are at the 3′-end of an oligonucleotide of an oligomeric compound. In certain embodiments, conjugate groups are near the 3′-end. In certain embodiments, conjugates are attached at the 3′end of an oligomeric compound, but before one or more terminal group nucleosides. In certain embodiments, conjugate groups are placed within a terminal group.

In certain embodiments, the present invention provides oligomeric compounds. In certain embodiments, oligomeric compounds comprise an oligonucleotide. In certain embodiments, an oligomeric compound comprises an oligonucleotide and one or more conjugate and/or terminal groups. Such conjugate and/or terminal groups may be added to oligonucleotides having any of the motifs discussed above. Thus, for example, an oligomeric compound comprising an oligonucleotide having region of alternating nucleosides may comprise a terminal group.

### C. Antisense Compounds

In certain embodiments, oligomeric compounds provided herein are antisense compounds. Such antisense compounds are capable of hybridizing to a target nucleic acid, resulting in at least one antisense activity. In certain embodiments, antisense compounds specifically hybridize to one or more target nucleic acid. In certain embodiments, a specifically hybridizing antisense compound has a nucleobase sequence comprising a region having sufficient complementarity to a target nucleic acid to allow hybridization and result in antisense activity and insufficient complementarity to any non-target so as to avoid non-specific hybridization to any non-target nucleic acid sequences under conditions in which specific hybridization is desired (e.g., under physiological conditions for in vivo or therapeutic uses, and under conditions in which assays are performed in the case of in vitro assays).

In certain embodiments, the present invention provides antisense compounds comprising oligonucleotides that are fully complementary to the target nucleic acid over the entire length of the oligonucleotide. In certain embodiments, oligonucleotides are 99% complementary to the target nucleic acid. In certain embodiments, oligonucleotides are 95% complementary to the target nucleic acid. In certain embodiments, such oligonucleotides are 90% complementary to the target nucleic acid.

In certain embodiments, such oligonucleotides are 85% complementary to the target nucleic acid. In certain embodiments, such oligonucleotides are 80% complementary to the target nucleic acid. In certain embodiments, an antisense compound comprises a region that is fully complementary to a target nucleic acid and is at least 80% complementary to the target nucleic acid over the entire length of the oligonucleotide. In certain such embodiments, the region of full complementarity is from 6 to 14 nucleobases in length.

a. Certain Antisense Activities and Mechanisms

In certain antisense activities, hybridization of an antisense compound results in recruitment of a protein that cleaves of the target nucleic acid. For example, certain antisense compounds result in RNase H mediated cleavage of target nucleic acid. RNase H is a cellular endonuclease that cleaves the RNA strand of an RNA:DNA duplex. The “DNA” in such an RNA:DNA duplex, need not be unmodified DNA. In certain embodiments, the invention provides antisense compounds that are sufficiently “DNA-like” to elicit RNase H activity. Such DNA-like antisense compounds include, but are not limited to gapmers having unmodified deoxyfuronose sugar moieties in the nucleosides of the gap and modified sugar moieties in the nucleosides of the wings.

Antisense activities may be observed directly or indirectly. In certain embodiments, observation or detection of an antisense activity involves observation or detection of a change in an amount of a target nucleic acid or protein encoded by such target nucleic acid; a change in the ratio of splice variants of a nucleic acid or protein; and/or a phenotypic change in a cell or animal.

In certain embodiments, compounds comprising oligonucleotides having a gapmer nucleoside motif described herein have desirable properties compared to non-gapmer oligonucleotides or to gapmers having other motifs. In certain circumstances, it is desirable to identify motifs resulting in a favorable combination of potent antisense activity and relatively low toxicity. In certain embodiments, compounds of the present invention have a favorable therapeutic index (measure of activity divided by measure of toxicity).

b. Certain Selective Antisense Compounds

In certain embodiments, antisense compounds provided are selective for a target relative to a non-target nucleic acid. In certain embodiments, the nucleobase sequences of the target and non-target nucleic acids differ by no more than 4 differentiating nucleobases in the targeted region. In certain embodiments, the nucleobase sequences of the target and non-target nucleic acids differ by no more than 3 differentiating nucleobases in the targeted region. In certain embodiments, the nucleobase sequences of the target and non-target nucleic acids differ by no more than 2 differentiating nucleobases in the targeted region. In certain embodiments, the nucleobase sequences of the target and non-target nucleic acids differ by a single differentiating nucleobase in the targeted region. In certain embodiments, the target and non-target nucleic acids are transcripts from different genes. In certain embodiments, the target and non-target nucleic acids are different alleles for the same gene. In certain embodiments, the introduction of a mismatch between an antisense compound and a non-target nucleic acid may alter the RNase H cleavage site of a target nucleic acid compared to a non-target nucleic acid. In certain embodiments, the target and non-target nucleic acids are not functionally related to one another (e.g., are transcripts from different genes). In certain embodiments, the target and not-target nucleic acids are allelic variants of one another. In certain embodiments, the allelic variant contains a single nucleotide polymorphism (SNP). In certain embodiments, a SNP is associated with a mutant allele. In certain embodiments, a mutant SNP is associated with a disease. In certain embodiments a mutant SNP is associated with a disease, but is not causative of the disease. In certain embodiments, mRNA and protein expression of a mutant allele is associated with disease.

Selectivity of antisense compounds is achieved, principally, by nucleobase complementarity. For example, if an antisense compound has no mismatches for a target nucleic acid and one or more mismatches for a non-target nucleic acid, some amount of selectivity for the target nucleic acid will result. In certain embodiments, provided herein are antisense compounds with enhanced selectivity (i.e. the ratio of activity for the target to the activity for non-target is greater). For example, in certain embodiments, a selective nucleoside comprises a particular feature or combination of features (e.g., chemical modification, motif, placement of selective nucleoside, and/or self-complementary region) that increases selectivity of an antisense compound compared to an antisense compound not having that feature or combination of features. In certain embodiments, such feature or combination of features increases antisense activity for the target. In certain embodiments, such feature or combination of features decreases activity for the target, but decreases activity for the non-target by a greater amount, thus resulting in an increase in selectivity.

Without being limited by mechanism, enhanced selectivity may result from a larger difference in the affinity of an antisense compound for its target compared to its affinity for the non-target and/or a larger difference in RNase H activity for the resulting duplexes. For example, in certain embodiments, a selective antisense compound comprises a modified nucleoside at that same position as a differentiating nucleobase (i.e., the selective nucleoside is modified). That modification may increase the difference in binding affinity of the antisense compound for the target relative to the non-target. In addition or in the alternative, the chemical modification may increase the difference in RNAse H activity for the duplex formed by the antisense compound and its target compared to the RNase activity for the duplex formed by the antisense compound and the non-target. For example, the modification may exaggerate a structure that is less compatible for RNase H to bind, cleave and/or release the non-target.

In certain embodiments, an antisense compound binds its intended target to form a target duplex. In certain embodiments, RNase H cleaves the target nucleic acid of the target duplex. In certain such embodiments, there is a primary cleavage site between two particular nucleosides of the target nucleic acid (the primary target cleavage site), which accounts for the largest amount of cleavage of the target nucleic acid. In certain embodiments, there are one or more secondary target cleavage sites. In certain embodiments, the same antisence compound hybridizes to a non-target to form a non-target duplex. In certain such embodiments, the non-target differs from the target by a single nucleobase within the target region, and so the antisense compound hybridizes with a single mismatch. Because of the mismatch, in certain embodiments, RNase H cleavage of the non-target may be reduced compared to cleavage of the target, but still occurs. In certain embodiments, though, the primary site of that cleavage of the non-target nucleic acid (primary non-target cleavage site) is different from that of the target. That is; the primary site is shifted due to the mismatch. In such a circumstance, one may use a modification placed in the antisense compound to disrupt RNase H cleavage at the primary non-target cleavage site. Such modification will result in reduced cleavage of the non-target, but will result little or no decrease in cleavage of the target. In certain embodiments, the modification is a modified sugar, nucleobase and/or linkage.

In certain embodiments, the primary non-target cleavage site is towards the 5′-end of the antisense compound, and the 5′-end of an antisense compound may be modified to prevent RNaseH cleavage. In this manner, it is thought that one having skill in the art may modify the 5′-end of an antisense compound, or modify the nucleosides in the gap region of the 5′-end of the antisense compound, or modify the 3′-most 5′-region nucleosides of the antisense compound to selectively inhibit RNaseH cleavage of the non-target nucleic acid duplex while retaining RNase H cleavage of the target nucleic acid duplex. In certain embodiments, 1-3 of the 3′-most 5′-region nucleosides of the antisense compound comprises a bicyclic sugar moiety.

For example, in certain embodiments the target nucleic acid may have an allelic variant, e.g. a non-target nucleic acid, containing a single nucleotide polymorphism. An antisense compound may be designed having a single nucleobase mismatch from the non-target nucleic acid, but which has full complementarity to the target nucleic acid. The mismatch between the antisense compound and the non-target nucleic acid may destabilize the antisense compound non-target nucleic acid duplex, and consequently the cleavage site of RNaseH may shift upstream towards the 5′-end of the antisense compound. Modification of the 5′-end of the antisense compound or the gap region near the 5′-end of the antisense compound, or one or more of the 3′-most nucleosides of the 5′-wing region, will then prevent RNaseH cleavage of the non-target nucleic acid. Since the target nucleic acid is fully complementary to the antisense compound, the antisense compound and the target nucleic acid will form a more stabilized antisense compound-target nucleic acid duplex and the cleavage site of RnaseH will be more downstream, towards the 3′ end of the antisense compound. Accordingly, modifications at the 5′-end of the antisense compound will prevent RNaseH cleavage of the non-target nucleic acid, but will not substantially effect RNaseH cleavage of the target nucleic acid, and selectivity between a target nucleic acid and its allelic variant may be achieved. In certain embodiments, one or more of the 3′-most nucleosides of the 5′-wing region comprises a bicyclic sugar moiety. In certain embodiments, one or more of the 3′-most nucleosides of the 5′-wing region comprises a bicyclic sugar moiety selected from cEt and LNA. In certain embodiments, one or more of the 3′-most nucleosides of the 5′-wing region comprises cEt. In certain embodiments, one or more of the 3′-most nucleosides of the 5′-wing region comprises LNA.

In certain embodiments, the introduction of a mismatch between an antisense compound and a target nucleic acid may alter the RNase H cleavage site of a target nucleic acid compared to a non-target nucleic acid by shifting the RNaseH cleavage site downstream from the mismatch site and towards the 3′-end of the antisense compound. In certain embodiments where the cleavage site of a target nucleic acid compared to a non-target nucleic acid has shifted downstream towards the 3′-end of the antisense compound, the 3′-end of an antisense compound may be modified to prevent RNaseH cleavage. In this manner, it is thought that one having skill in the art may modify the 3′-end of an antisense compound, or modify the nucleosides in the gap region near the 3′-end of antisense compound, to selectively inhibit RNaseH cleavage of the non-target nucleic acid while retaining RNase H cleavage of the target nucleic acid.

For example, in certain embodiments the target nucleic acid may have an allelic variant, e.g. a non-target nucleic acid, containing a single nucleotide polymorphism. An antisense compound may be designed having a single nucleobase mismatch from the non-target nucleic acid, but which has full complementarity to target nucleic acid. The mismatch between the antisense compound and the non-target nucleic acid may destabilize the antisense compound-non-target nucleic acid duplex, and consequently the cleavage site of RNaseH may shift downstream towards the 3′-end of the antisense compound. Modification of the 3′-end of the antisense compound, or one or more of the 5′-most nucleosides of the 3′-wing region, or the gap region of the antisense compound near the 3′-end will then prevent RNaseH cleavage of the non-target nucleic acid. Since the target nucleic acid is fully complementary to the antisense compound, the antisense compound and the target nucleic acid will form a more stabilized antisense compound-target nucleic acid duplex and the cleavage site of RnaseH will be more upstream, towards the 5′ end of the antisense compound. Accordingly, modifications at the 3′-end of the antisense compound will prevent RNaseH cleavage of the non-target nucleic acid, but will not substantially effect RNaseH cleavage of the target nucleic acid, and selectivity between a target nucleic acid and its allelic variant may be achieved. In certain embodiments, one or more of the 5′-most nucleosides of the 3′-wing region comprises a bicyclic sugar moiety. In certain embodiments, one or more of the 5′-most nucleosides of the 3′-wing region comprises a bicyclic sugar moiety selected from cEt and LNA. In certain embodiments, one or more of the 5′-most nucleosides of the 3′-wing region comprises cEt. In certain embodiments, one or more of the 5′-most nucleosides of the 3′-wing region comprises LNA.

In certain embodiments, the selectivity of antisense compounds having certain gaps, e.g. gaps of 7 nucleosides or longer, may be improved by the addition of one or more bicyclic nucleosides at the 3′-most 5′-wing nucleoside. In certain embodiments, the selectivity of antisense compounds having certain gaps, e.g. gaps of 7 nucleosides or longer, may be improved by the addition of two or more bicyclic nucleosides at the 3′-most 5′-wing nucleoside. In certain embodiments, the selectivity of antisense compounds having certain gaps, e.g. gaps of 7 nucleosides or longer, may be improved by the addition of one bicyclic nucleoside at the 3′-most 5′-wing nucleoside. In certain embodiments, the selectivity of antisense compounds having certain gaps, e.g. gaps of 7 nucleosides or longer, may be improved by the addition of two bicyclic nucleosides at the 3′-most 5′-wing nucleoside. In certain embodiments, the selectivity of antisense compounds having certain gaps, e.g. gaps of 7 nucleosides or longer, may be improved by the addition of three bicyclic nucleosides at the 3′-most 5′-wing nucleoside. In certain embodiments, the selectivity of antisense compounds having certain gaps, e.g. gaps of 7 nucleosides or longer, may be improved by the addition of four bicyclic nucleosides at the 3′-most 5′-wing nucleoside. In certain embodiments, the selectivity of antisense compounds having certain gaps, e.g. gaps of 7 nucleosides or longer, may be improved by the addition of five bicyclic nucleosides at the 3′-most 5′-wing nucleoside. In certain embodiments discussed above, the bicyclic nucleosides at the 3′-most 5′-wing nucleoside are selected from among cEt, cMOE, LNA, α-LNA, ENA and 2′-thio LNA. In certain embodiments discussed above, the bicyclic nucleosides at the 3′-most 5′-wing nucleoside comprise cEt. In certain embodiments discussed above, the bicyclic nucleosides at the 3′-most 5′-wing nucleoside comprise LNA.

In certain embodiments, the selectivity of antisense compounds having certain gaps, e.g. gaps of 7 nucleosides or longer, may be improved by the addition of one or more bicyclic nucleosides at the 3′-most 5′-wing nucleoside and the addition of one or more bicyclic nucleosides at the 5′-most 3′-wing nucleoside. In certain embodiments, the selectivity of antisense compounds having certain gaps, e.g. gaps of 7 nucleosides or longer, may be improved by the addition of two or more bicyclic nucleosides at the 3′-most 5′-wing nucleoside and the addition of one or more bicyclic nucleosides at the 5′-most 3′-wing nucleoside. In certain embodiments, the selectivity of antisense compounds having certain gaps, e.g. gaps of 7 nucleosides or longer, may be improved by the addition of one bicyclic nucleoside at the 3′-most 5′-wing nucleoside and the addition of one or more bicyclic nucleosides at the 5′-most 3′-wing nucleoside. In certain embodiments, the selectivity of antisense compounds having certain gaps, e.g. gaps of 7 nucleosides or longer, may be improved by the addition of two bicyclic nucleosides at the 3′-most 5′-wing nucleoside and the addition of one or more bicyclic nucleosides at the 5′-most 3′-wing nucleoside. In certain embodiments, the selectivity of antisense compounds having certain gaps, e.g. gaps of 7 nucleosides or longer, may be improved by the addition of three bicyclic nucleosides at the 3′-most 5′-wing nucleoside and the addition of one or more bicyclic nucleosides at the 5′-most 3′-wing nucleoside. In certain embodiments, the selectivity of antisense compounds having certain gaps, e.g. gaps of 7 nucleosides or longer, may be improved by the addition of four bicyclic nucleosides at the 3′-most 5′-wing nucleoside and the addition of one or more bicyclic nucleosides at the 5′-most 3′-wing nucleoside. In certain embodiments, the selectivity of antisense compounds having certain gaps, e.g. gaps of 7 nucleosides or longer, may be improved by the addition of four bicyclic nucleosides at the 3′-most 5′-wing nucleoside and the addition of one or more bicyclic nucleosides at the 5′-most 3′-wing nucleoside.

In certain embodiments, the selectivity of antisense compounds having certain gaps, e.g. gaps of 7 nucleosides or shorter, may be improved by the addition of one or more bicyclic nucleosides at the 3′-most 5′-wing nucleoside. In certain embodiments, the selectivity of antisense compounds having certain gaps, e.g. gaps of 7 nucleosides or shorter, may be improved by the addition of two or more bicyclic nucleosides at the 3′-most 5′-wing nucleoside. In certain embodiments, the selectivity of antisense compounds having certain gaps, e.g. gaps of 7 nucleosides or shorter, may be improved by the addition of one bicyclic nucleoside at the 3′-most 5′-wing nucleoside. In certain embodiments, the selectivity of antisense compounds having certain gaps, e.g. gaps of 7 nucleosides or shorter, may be improved by the addition of two bicyclic nucleosides at the 3′-most 5′-wing nucleoside. In certain embodiments, the selectivity of antisense compounds having certain gaps, e.g. gaps of 7 nucleosides or shorter, may be improved by the addition of three bicyclic nucleosides at the 3′-most 5′-wing nucleoside. In certain embodiments, the selectivity of antisense compounds having certain gaps, e.g. gaps of 7 nucleosides or shorter, may be improved by the addition of four bicyclic nucleosides at the 3′-most 5′-wing nucleoside. In certain embodiments, the selectivity of antisense compounds having certain gaps, e.g. gaps of 7 nucleosides or shorter, may be improved by the addition of five bicyclic nucleosides at the 3′-most 5′-wing nucleoside. In certain embodiments discussed above, the bicyclic nucleosides at the 3′-most 5′-wing nucleoside are selected from among cEt, cMOE, LNA, α-LNA, ENA and 2′-thio LNA. In certain embodiments discussed above, the bicyclic nucleosides at the 3′-most 5′-wing nucleoside comprise cEt. In certain embodiments discussed above, the bicyclic nucleosides at the 3′-most 5′-wing nucleoside comprise LNA.

Antisense compounds having certain specified motifs have enhanced selectivity, including, but not limited to motifs described above. In certain embodiments, enhanced selectivity is achieved by oligonucleotides comprising any one or more of:
a modification motif comprising a long 5′-wing (longer than 5, 6, or 7 nucleosides);
a modification motif comprising a long 3′-wing (longer than 5, 6, or 7 nucleosides);
a modification motif comprising a short gap region (shorter than 8, 7, or 6 nucleosides); and
a modification motif comprising an interrupted gap region (having no uninterrupted stretch of unmodified 2′-deoxynucleosides longer than 7, 6 or 5).

i. Certain Selective Nucleobase Sequence Elements

In certain embodiments, selective antisense compounds comprise nucleobase sequence elements. Such nucleobase sequence elements are independent of modification motifs. Accordingly, oligonucleotides having any of the motifs (modification motifs, nucleoside motifs, sugar motifs, nucleobase modification motifs, and/or linkage motifs) may also comprise one or more of the following nucleobase sequence elements.

ii. Alignment of Differentiating Nucleobase/Target-Selective Nucleoside

In certain embodiments, a target region and a region of a non-target nucleic acid differ by 1-4 differentiating nucleobase. In such embodiments, selective antisense compounds have a nucleobase sequence that aligns with the non-target nucleic acid with 1-4 mismatches. A nucleoside of the antisense compound that corresponds to a differentiating nucleobase of the target nucleic acid is referred to herein as a target-selective nucleoside. In certain embodiments, selective antisense compounds having a gapmer motif align with a non-target nucleic acid, such that a target-selective nucleoside is positioned in the gap. In certain embodiments, a target-selective nucleoside is the 1^{st} nucleoside of the gap from the 5′ end. In certain embodiments, a target-selective nucleoside is the 2^{nd} nucleoside of the gap from the 5′ end. In certain embodiments, a target-selective nucleoside is the 3^{rd} nucleoside of the gap from the 5′-end. In certain embodiments, a target-selective nucleoside is the 4^{th} nucleoside of the gap from the 5′-end. In certain embodiments, a target-selective nucleoside is the 5^{th} nucleoside of the gap from the 5′-end. In certain embodiments, a target-selective nucleoside is the 6^{rd} nucleoside of the gap from the 5′-end. In certain embodiments, a target-selective nucleoside is the 8^{th} nucleoside of the gap from the 3′-end. In certain embodiments, a target-selective nucleoside is the 7^{th} nucleoside of the gap from the 3′-end. In certain embodiments, a target-selective nucleoside is the 6^{th} nucleoside of the gap from the 3′-end. In certain embodiments, a target-selective nucleoside is the 5^{th} nucleoside of the gap from the 3′-end. In certain embodiments, a target-selective nucleoside is the 4^{th} nucleoside of the gap from the 3′-end. In certain embodiments, a target-selective nucleoside is the 3^{rd} nucleoside of the gap from the 3′-end. In certain embodiments, a target-selective nucleoside is the 2^{nd} nucleoside of the gap from the 3′-end.

In certain embodiments, a target-selective nucleoside comprises a modified nucleoside. In certain embodiments, a target-selective nucleoside comprises a modified sugar. In certain embodiments, a target-selective nucleoside comprises a sugar surrogate. In certain embodiments, a target-selective nucleoside comprises a sugar surrogate selected from among HNA and F-HNA. In certain embodiments, a target-selective nucleoside comprises a 2′-substituted sugar moiety. In certain embodiments, a target-selective nucleoside comprises a 2′-substituted sugar moiety selected from among MOE, F and (ara)-F. In certain embodiments, a target-selective nucleoside comprises a 5′-substituted sugar moiety. In certain embodiments, a target-selective nucleoside comprises a 5′-substituted sugar moiety selected from 5′-(R)-Me DNA. In certain embodiments, a target-selective nucleoside comprises a bicyclic sugar moiety. In certain embodiments, a target-selective nucleoside comprises a bicyclic sugar moiety selected from among cEt, and α-L-LNA. In certain embodiments, a target-selective nucleoside comprises a modified nucleobase. In certain embodiments, a target-selective nucleoside comprises a modified nucleobase selected from among 2-thio-thymidine and 5-propyne uridine.

iii. Mismatches to the Target Nucleic Acid

In certain embodiments, selective antisense compounds comprise one or more mismatched nucleobases relative to the target nucleic acid. In certain such embodiments, antisense activity against the target is reduced by such mismatch, but activity against the non-target is reduced by a greater amount. Thus, in certain embodiments selectivity is improved. Any nucleobase other than the differentiating nucleobase is suitable for a mismatch. In certain embodiments, however, the mismatch is specifically positioned within the gap of an oligonucleotide having a gapmer motif. In certain embodiments, a mismatch relative to the target nucleic acid is at positions 1, 2, 3, 4, 5, 6, 7, or 8 from the 5′-end of the gap region. In certain embodiments, a mismatch relative to the target nucleic acid is at positions 9, 8, 7, 6, 5, 4, 3, 2, 1 of the antisense compounds from the 3′-end of the gap region. In certain embodiments, a mismatch relative to the target nucleic acid is at positions 1, 2, 3, or 4 of the antisense compounds from the 5′-end of the wing region. In certain embodiments, a mismatch relative to the target nucleic acid is at positions 4, 3, 2, or 1 of the antisense compounds from the 3′-end of the wing region.

iv. Self Complementary Regions

In certain embodiments, selective antisense compounds comprise a region that is not complementary to the target. In certain embodiments, such region is complementary to another region of the antisense compound. Such regions are referred to herein as self-complementary regions. For example, in certain embodiments, an antisense compound has a first region at one end that is complementary to a second region at the other end. In certain embodiments, one of the first and second regions is complementary to the target nucleic acid. Unless the target nucleic acid also includes a self-complementary region, the other of the first and second region of the antisense compound will not be complementary to the target nucleic acid. For illustrative purposes, certain antisense compounds have the following nucleobase motif:

ABCXXXXXXXXXC′B′A′;
ABCXXXXXXX(X/C′)(X/B′)(X/A′);
(X/A)(X/B)(X/C)XXXXXXXXXC′B′A′

where each of A, B, and C are any nucleobase; A′, B′, and C′ are the complementary bases to A, B, and C, respectively; each X is a nucleobase complementary to the target nucleic acid; and two letters in parentheses (e.g., (X/C′)) indicates that the nucleobase is complementary to the target nucleic acid and to the designated nucleoside within the antisense oligonucleotide.

Without being bound to any mechanism, in certain embodiments, such antisense compounds are expected to form self-structure, which is disrupted upon contact with a target nucleic acid. Contact with a non-target nucleic acid is expected to disrupt the self-structure to a lesser degree, thus increasing selectivity compared to the same antisense compound lacking the self-complementary regions.

v. Combinations of Features

Though it is clear to one of skill in the art, the above motifs and other elements for increasing selectivity may be used alone or in combination. For example, a single antisense compound may include any one, two, three, or more of self-complementary regions, a mismatch relative to the target nucleic acid, a short nucleoside gap, an interrupted gap, and specific placement of the selective nucleoside.

### D. Certain Short Gap Antisense Compounds

In certain embodiments, an antisense compound of interest may modulate the expression of a target nucleic acid but possess undesirable properties. In certain embodiments, for example, an antisense compound of interest may have an undesirably high affinity for one or more non-target nucleic acids. In certain embodiments, whether as a result of such affinity for one or more non-target nucleic acid or by some other mechanism, an antisense compound of interest may produce undesirable increases in ALT and/or AST levels when administered to an animal. In certain embodiments, such an antisense compound of interest may produce undesirable increases in organ weight.

In certain such embodiments wherein an antisense compound of interest effectively modulates the expression of a target nucleic acid, but possess one or more undesirable properties, a person having skill in the art may selectively incorporate one or more modifications into the antisense compound of interest that retain some or all of the desired property of effective modulation of expression of a target nucleic acid while reducing one or more of the antisense compound's undesirable properties. In certain embodiments, the present invention provides methods of altering such an antisense compound of interest to form an improved antisense compound. In certain embodiments, altering the number of nucleosides in the 5′-region, the 3′-region, and/or the central region of such an antisense compound of interest results in improved properties. For example, in certain embodiments, one may alter the modification state of one or more nucleosides at or near the 5′-end of the central region. Having been altered, those nucleosides may then be characterized as being part of the 5′-region. Thus, in such embodiments, the overall number of nucleosides of the 5′-region is increased and the number of nucleosides in the central region is decreased. For example, an antisense compound having a modification motif of 3-10-3 could be altered to result in an improved antisense compound having a modification motif of 4-9-3 or 5-8-3. In certain embodiments, the modification state of one or more of nucleosides at or near the 3′-end of the central region may likewise be altered. In certain embodiments, the modification of one or more of the nucleosides at or near the 5′-end and the 3′-end of the central region may be altered. In such embodiments in which one or more nucleosides at or near the 5′-end and the 3′-end of the central region is altered the central region becomes shorter relative to the central region of the original antisense compound of interest. In such embodiments, the modifications to the one or more nucleosides that had been part of the central region are the same as one or more modification that had been present in the 5′-region and/or the 3′-region of the original antisense compound of interest. In certain embodiments, the improved antisense compound having a shortened central region may retain its ability to effectively modulate the expression of a target nucleic acid, but not possess some or all of the undesirable properties possessed by antisense compound of interest having a longer central region. In certain embodiments, reducing the length of the central region reduces affinity for off-target nucleic acids. In certain embodiments, reducing the length of the central region results in reduced cleavage of non-target nucleic acids by RNase H. In certain embodiments, reducing the length of the central region does not produce undesirable increases in ALT levels. In certain embodiments, reducing the length of the central region does not produce undesirable increases in AST levels. In certain embodiments, reducing the length of the central region does not produce undesirable increases organ weights.

In certain embodiments it is possible to retain the same nucleobase sequence and overall length of an antisense compound while decreasing the length of the central region. In certain embodiments retaining the same nucleobase sequence and overall length of an antisense compound while decreasing the length of the central region ameliorates one or more undesirable properties of an antisense compound. In certain embodiments retaining the same nucleobase sequence and overall length of an antisense compound while decreasing the length of the central region ameliorates one or more undesirable properties of an antisense compound but does not substantially affect the ability of the antisense compound to modulate expression of a target nucleic acid. In certain such embodiments, two or more antisense compounds would have the same overall length and nucleobase sequence, but would have a different central region length, and different properties. In certain embodiments, the length of the central region is 9 nucleobases. In certain embodiments, the length of the central region is 8 nucleobases. In certain embodiments, the length of the central region is 7 nucleobases. In certain embodiments, the central region consists of unmodified deoxynucleosides. In certain embodiments, the length of the central region can be decreased by increasing the length of the 5′-region, the 3′-region, or both the 5′-region and the 3′-region.

In certain embodiments, the length of the central region can be decreased by increasing the length of the 5′-region with modified nucleosides. In certain embodiments, the length of the central region can be decreased by increasing the length of the 5′-region with modified nucleosides. In certain embodiments, the length of the central region can be decreased by increasing the length of the 5′-region with modified nucleosides comprising a bicyclic sugar moiety selected from among: cEt, cMOE, LNA, α-LNA, ENA and 2′-thio LNA. In certain embodiments, the length of the central region can be decreased by increasing the length of the 5′-region with a cEt substituted sugar moiety.

In certain embodiments, the length of the central region can be decreased by increasing the length of the 5′-region with modified nucleosides. In certain embodiments, the length of the central region can be decreased by increasing the length of the 5′-region with modified nucleosides. In certain embodiments, the length of the central region can be decreased by increasing the length of the 5′-region with modified nucleosides comprising a bicyclic sugar moiety comprising a 2′ substituent selected from among: a halogen, OCH₃, OCF₃, OCH₂CH₃, OCH₂CF₃, OCH₂—CH═CH₂, O(CH₂)₂—OCH₃ (MOE), O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂. In certain embodiments, the length of the central region can be decreased by increasing the length of the 5′-region with 2′-O(CH₂)₂—OCH₃ (MOE) substituted sugar moiety.

In certain embodiments, the length of the central region can be decreased by increasing the length of the 3′-region with modified nucleosides. In certain embodiments, the length of the central region can be decreased by increasing the length of the 3′-region with modified nucleosides. In certain embodiments, the length of the central region can be decreased by increasing the length of the 3′-region with modified nucleosides comprising a bicyclic sugar moiety selected from among: cEt, cMOE, LNA, α-LNA, ENA and 2′-thio LNA. In certain embodiments, the length of the central region can be decreased by increasing the length of the 3′-region with a cEt substituted sugar moiety.

In certain embodiments, the length of the central region can be decreased by increasing the length of the 3′-region with modified nucleosides. In certain embodiments, the length of the central region can be decreased by increasing the length of the 3′-region with modified nucleosides. In certain embodiments, the length of the central region can be decreased by increasing the length of the 3′-region with modified nucleosides comprising a bicyclic sugar moiety comprising a 2′ substituent selected from among: a halogen, OCH₃, OCF₃, OCH₂CH₃, OCH₂CF₃, OCH₂—CH═CH₂, O(CH₂)₂—OCH₃ (MOE), O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂. In certain embodiments, the length of the central region can be decreased by increasing the length of the 3′-region with 2′-O(CH₂)₂—OCH₃ (MOE) substituted sugar moiety.

In certain embodiments, the length of the central region can be decreased by increasing the length of the 5′-region with modified nucleosides and increasing the length of the 3′-region with modified nucleosides.

### E. Certain Target Nucleic Acids

In certain embodiments, antisense compounds comprise or consist of an oligonucleotide comprising a region that is complementary to a target nucleic acid. In certain embodiments, the target nucleic acid is an endogenous RNA molecule. In certain embodiments, the target nucleic acid is a non-coding RNA. In certain such embodiments, the target non-coding RNA is selected from: a long-non-coding RNA, a short non-coding RNA, an intronic RNA molecule, a snoRNA, a scaRNA, a microRNA (including pre-microRNA and mature microRNA), a ribosomal RNA, and promoter directed RNA. In certain embodiments, the target nucleic acid encodes a protein. In certain such embodiments, the target nucleic acid is selected from: an mRNA and a pre-mRNA, including intronic, exonic and untranslated regions. In certain embodiments, oligomeric compounds are at least partially complementary to more than one target nucleic acid. For example, antisense compounds of the present invention may mimic microRNAs, which typically bind to multiple targets.

In certain embodiments, the target nucleic acid is a nucleic acid other than a mature mRNA. In certain embodiments, the target nucleic acid is a nucleic acid other than a mature mRNA or a microRNA. In certain embodiments, the target nucleic acid is a non-coding RNA other than a microRNA. In certain embodiments, the target nucleic acid is a non-coding RNA other than a microRNA or an intronic region of a pre-mRNA. In certain embodiments, the target nucleic acid is a long non-coding RNA. In certain embodiments, the target RNA is an mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron. In certain embodiments, the target nucleic acid is selected from among non-coding RNA, including exonic regions of pre-mRNA. In certain embodiments, the target nucleic acid is a ribosomal RNA (rRNA). In certain embodiments, the target nucleic acid is a non-coding RNA associated with splicing of other pre-mRNAs. In certain embodiments, the target nucleic acid is a nuclear-retained non-coding RNA.

In certain embodiments, antisense compounds described herein are complementary to a target nucleic acid comprising a single-nucleotide polymorphism. In certain such embodiments, the antisense compound is capable of modulating expression of one allele of the single-nucleotide polymorphism-containing-target nucleic acid to a greater or lesser extent than it modulates another allele. In certain embodiments an antisense compound hybridizes to a single-nucleotide polymorphism-containing-target nucleic acid at the single-nucleotide polymorphism site. In certain embodiments, the target nucleic acid is a Huntingtin gene transcript. In certain embodiments, the target nucleic acid is a single-nucleotide polymorphism-containing-target nucleic acid of a Huntingtin gene transcript. In certain embodiments, the target nucleic acid is not a Huntingtin gene transcript. In certain embodiments, the target nucleic acid is a single-nucleotide polymorphism-containing-target nucleic acid of a gene transcript other than Huntingtin. In certain embodiments, the target nucleic acid is any nucleic acid other than a Huntingtin gene transcript.

a. Single-Nucleotide Polymorphism

In certain embodiments, the invention provides selective antisense compounds that have greater activity for a target nucleic acid than for a homologous or partially homologous non-target nucleic acid. In certain such embodiments, the target and non-target nucleic acids are not functionally related to one another (e.g., are transcripts from different genes). In certain embodiments, the target and not-target nucleic acids are allelic variants of one another. Certain embodiments of the present invention provide methods, compounds, and compositions for selectively inhibiting mRNA and protein expression of an allelic variant of a particular gene or DNA sequence. In certain embodiments, the allelic variant contains a single nucleotide polymorphism (SNP). In certain embodiments, a SNP is associated with a mutant allele. In certain embodiments, a mutant SNP is associated with a disease. In certain embodiments a mutant SNP is associated with a disease, but is not causative of the disease. In certain embodiments, mRNA and protein expression of a mutant allele is associated with disease.

In certain embodiments, the expressed gene product of a mutant allele results in aggregation of the mutant proteins causing disease. In certain embodiments, the expressed gene product of a mutant allele results in gain of function causing disease. In certain embodiments, genes with an autosomal dominant mutation resulting in a toxic gain of function of the protein are the APP gene encoding amyloid precursor protein involved in Alzheimer's disease (Gene, 371: 68, 2006); the PrP gene encoding prion protein involved in Creutzfeldt-Jakob disease and in fatal familial insomnia (Nat. Med. 1997, 3: 1009); GFAP gene encoding glial fibrillary acidic protein involved in Alexander disease (J. Neurosci. 2006, 26:111623); alpha-synuclein gene encoding alpha-synuclein protein involved in Parkinson's disease (J. Clin. Invest. 2003, 111: 145); SOD-1 gene encoding the SOD-1 protein involved in amyotrophic lateral sclerosis (Science 1998, 281: 1851); atrophin-1 gene encoding atrophin-1 protein involved in dentato-rubral and pallido-luysian atrophy (DRPA) (Trends Mol. Med. 2001, 7: 479); SCA1 gene encoding ataxin-1 protein involved in spino-cerebellar ataxia-1 (SCA1) (Protein Sci. 2003, 12: 953); PLP gene encoding proteolipid protein involved in Pelizaeus-Merzbacher disease (NeuroMol Med. 2007, 4: 73); DYT1 gene encoding torsinA protein involved in Torsion dystonia (Brain Res. 2000, 877: 379); and alpha-B crystalline gene encoding alpha-B crystalline protein involved in protein aggregation diseases, including cardiomyopathy (Cell 2007, 130: 427); alpha1-antitrypsin gene encoding alpha1-antitrypsin protein involved in chronic obstructive pulmonary disease (COPD), liver disease and hepatocellular carcinoma (New Engl J Med. 2002, 346: 45); Ltk gene encoding leukocyte tyrosine kinase protein involved in systemic lupus erythematosus (Hum. Mol. Gen. 2004, 13: 171); PCSK9 gene encoding PCSK9 protein involved in hypercholesterolemia (Hum Mutat. 2009, 30: 520); prolactin receptor gene encoding prolactin receptor protein involved in breast tumors (Proc. Natl. Assoc. Sci. 2008, 105: 4533); CCL5 gene encoding the chemokine CCL5 involved in COPD and asthma (Eur. Respir. J. 2008, 32: 327); PTPN22 gene encoding PTPN22 protein involved in Type 1 diabetes, Rheumatoid arthritis, Graves disease, and SLE (Proc. Natl. Assoc. Sci. 2007, 104: 19767); androgen receptor gene encoding the androgen receptor protein involved in spinal and bulbar muscular atrophy or Kennedy's disease (J Steroid Biochem. Mol. Biol. 2008, 108: 245); CHMP4B gene encoding chromatin modifying protein-4B involved in progressive childhood posterior subcapsular cataracts (Am. J. Hum. Genet 2007, 81: 596); FXR/NR1H4 gene encoding Farnesoid X receptor protein involved in cholesterol gallstone disease, arthrosclerosis and diabetes (Mol. Endocrinol. 2007, 21: 1769); ABCA1 gene encoding ABCA1 protein involved in cardiovascular disease (Transl. Res. 2007, 149: 205); CaSR gene encoding the calcium sensing receptor protein involved in primary hypercalciuria (Kidney Int. 2007, 71: 1155); alpha-globin gene encoding alpha-globin protein involved in alpha-thallasemia (Science 2006, 312: 1215); httlpr gene encoding HTTLPR protein involved in obsessive compulsive disorder (Am. J. Hum. Genet. 2006, 78: 815); AVP gene encoding arginine vasopressin protein in stress-related disorders such as anxiety disorders and comorbid depression (CNS Neurol. Disord. Drug Targets 2006, 5: 167); GNAS gene encoding G proteins involved in congenital visual defects, hypertension, metabolic syndrome (Trends Pharmacol. Sci. 2006, 27: 260); APAF1 gene encoding APAF1 protein involved in a predisposition to major depression (Mol. Psychiatry 2006, 11: 76); TGF-beta1 gene encoding TGF-beta1 protein involved in breast cancer and prostate cancer (Cancer Epidemiol. Biomarkers Prev. 2004, 13: 759); AChR gene encoding acetylcholine receptor involved in congential myasthenic syndrome (Neurology 2004, 62: 1090); P2Y12 gene encoding adenosine diphosphate (ADP) receptor protein involved in risk of peripheral arterial disease (Circulation 2003, 108: 2971); LQT1 gene encoding LQT1 protein involved in atrial fibrillation (Cardiology 2003, 100: 109); RET protooncogene encoding RET protein involved in sporadic pheochromocytoma (J. Clin. Endocrinol. Metab. 2003, 88: 4911); filamin A gene encoding filamin A protein involved in various congenital malformations (Nat. Genet. 2003, 33: 487); TARDBP gene encoding TDP-43 protein involved in amyotrophic lateral sclerosis (Hum. Mol. Gene.t 2010, 19: 671); SCA3 gene encoding ataxin-3 protein involved in Machado-Joseph disease (PLoS One 2008, 3: e3341); SCA7 gene encoding ataxin-7 protein involved in spino-cerebellar ataxia-7 (PLoS One 2009, 4: e7232); and HTT gene encoding huntingtin protein involved in Huntington's disease (Neurobiol Dis. 1996, 3:183); and the CA4 gene encoding carbonic anhydrase 4 protein, CRX gene encoding cone-rod homeobox transcription factor protein, FSCN2 gene encoding retinal fascin homolog 2 protein, IMPDH1 gene encoding inosine monophosphate dehydrogenase 1 protein, NR2E3 gene encoding nuclear receptor subfamily 2 group E3 protein, NRL gene encoding neural retina leucine zipper protein, PRPF3 (RP18) gene encoding pre-mRNA splicing factor 3 protein, PRPF8 (RP13) gene encoding pre-mRNA splicing factor 8 protein, PRPF31 (RP11) gene encoding pre-mRNA splicing factor 31 protein, RDS gene encoding peripherin 2 protein, ROM1 gene encoding rod outer membrane protein 1 protein, RHO gene encoding rhodopsin protein, RP1 gene encoding RP1 protein, RPGR gene encoding retinitis pigmentosa GTPase regulator protein, all of which are involved in Autosomal Dominant Retinitis Pigmentosa disease (Adv Exp Med Biol. 2008, 613:203)

In certain embodiments, the mutant allele is associated with any disease from the group consisting of Alzheimer's disease, Creutzfeldt-Jakob disease, fatal familial insomnia, Alexander disease, Parkinson's disease, amyotrophic lateral sclerosis, dentato-rubral and pallido-luysian atrophy DRPA, spino-cerebellar ataxia, Torsion dystonia, cardiomyopathy, chronic obstructive pulmonary disease (COPD), liver disease, hepatocellular carcinoma, systemic lupus erythematosus, hypercholesterolemia, breast cancer, asthma, Type 1 diabetes, Rheumatoid arthritis, Graves disease, SLE, spinal and bulbar muscular atrophy, Kennedy's disease, progressive childhood posterior subcapsular cataracts, cholesterol gallstone disease, arthrosclerosis, cardiovascular disease, primary hypercalciuria, alpha-thallasemia, obsessive compulsive disorder, Anxiety, comorbid depression, congenital visual defects, hypertension, metabolic syndrome, prostate cancer, congential myasthenic syndrome, peripheral arterial disease, atrial fibrillation, sporadic pheochromocytoma, congenital malformations, Machado-Joseph disease, Huntington's disease, and Autosomal Dominant Retinitis Pigmentosa disease.

i. Certain Huntingtin Targets

In certain embodiments, an allelic variant of huntingtin is selectively reduced. Nucleotide sequences that encode huntingtin include, without limitation, the following: GENBANK Accession No. NT_006081.18, truncated from nucleotides 1566000 to 1768000 (replaced by GENBANK Accession No. NT_006051), incorporated herein as SEQ ID NO: 1, and NM_002111.6, incorporated herein as SEQ ID NO: 2.

Table 14 provides SNPs found in the GM04022, GM04281, GM02171, and GM02173B cell lines. Also provided are the allelic variants found at each SNP position, the genotype for each of the cell lines, and the percentage of HD patients having a particular allelic variant. For example, the two allelic variants for SNP rs6446723 are T and C. The GM04022 cell line is heterozygous TC, the GM02171 cell line is homozygous CC, the GM02173 cell line is heterozygous TC, and the GM04281 cell line is homozygous TT. Fifty percent of HD patients have a T at SNP position rs6446723.

TABLE 14
Allelic Variations for SNPs Associated with HD

| SNP | Variation | GM04022 | GM02171 | GM02173 | GM04281 | TargetPOP | allele |
|---|---|---|---|---|---|---|---|
| rs6446723 | T/C | TC | CC | TC | TT | 0.50 | T |
| rs3856973 | A/G | AG | AA | AG | GG | 0.50 | G |
| rs2285086 | A/G | AG | GG | AG | AA | 0.50 | A |
| rs363092 | A/C | AC | AA | AC | CC | 0.49 | C |
| rs916171 | C/G | GC | GG | GC | CC | 0.49 | C |
| rs6844859 | T/C | TC | CC | TC | TT | 0.49 | T |
| rs7691627 | A/G | AG | AA | AG | GG | 0.49 | G |
| rs4690073 | A/G | AG | AA | AG | GG | 0.49 | G |
| rs2024115 | A/G | AG | GG | AG | AA | 0.48 | A |
| rs11731237 | T/C | CC | CC | TC | TT | 0.43 | T |
| rs362296 | A/C | CC | AC | AC | AC | 0.42 | C |
| rs10015979 | A/G | AA | AA | AG | GG | 0.42 | G |
| rs7659144 | C/G | CG | CG | CG | CC | 0.41 | C |
| rs363096 | T/C | CC | CC | TC | TT | 0.40 | T |
| rs362273 | A/G | AA | AG | AG | AA | 0.39 | A |
| rs16843804 | T/C | CC | TC | TC | CC | 0.38 | C |
| rs362271 | A/G | GG | AG | AG | GG | 0.38 | G |
| rs362275 | T/C | CC | TC | TC | CC | 0.38 | C |
| rs3121419 | T/C | CC | TC | TC | CC | 0.38 | C |
| rs362272 | A/G | GG | — | AG | GG | 0.38 | G |
| rs3775061 | A/G | AA | AG | AG | AA | 0.38 | A |
| rs34315806 | T/C | CC | TC | TC | CC | 0.38 | C |
| rs363099 | T/C | CC | TC | TC | CC | 0.38 | C |
| rs2298967 | T/C | TT | TC | TC | TT | 0.38 | T |
| rs363088 | A/T | AA | TA | TA | AA | 0.38 | A |
| rs363064 | T/C | CC | TC | TC | CC | 0.35 | C |
| rs363102 | A/G | AG | AA | AA | AA | 0.23 | G |
| rs2798235 | A/G | AG | GG | GG | GG | 0.21 | A |
| rs363080 | T/C | TC | CC | CC | CC | 0.21 | T |
| rs363072 | A/T | TA | TA | AA | AA | 0.13 | A |
| rs363125 | A/C | AC | AC | CC | CC | 0.12 | C |
| rs362303 | T/C | TC | TC | CC | CC | 0.12 | C |
| rs362310 | T/C | TC | TC | CC | CC | 0.12 | C |
| rs10488840 | A/G | AG | AG | GG | GG | 0.12 | G |
| rs362325 | T/C | TC | TC | TT | TT | 0.11 | T |
| rs35892913 | A/G | GG | GG | GG | GG | 0.10 | A |
| rs363102 | A/G | AG | AA | AA | AA | 0.09 | A |
| rs363096 | T/C | CC | CC | TC | TT | 0.09 | C |
| rs11731237 | T/C | CC | CC | TC | TT | 0.09 | C |
| rs10015979 | A/G | AA | AA | AG | GG | 0.08 | A |
| rs363080 | T/C | TC | CC | CC | CC | 0.07 | C |
| rs2798235 | A/G | AG | GG | GG | GG | 0.07 | G |
| rs1936032 | C/G | GC | CC | CC | CC | 0.06 | C |
| rs2276881 | A/G | GG | GG | GG | GG | 0.06 | G |
| rs363070 | A/G | AA | AA | AA | AA | 0.06 | A |
| rs35892913 | A/G | GG | GG | GG | GG | 0.04 | G |
| rs12502045 | T/C | CC | CC | CC | CC | 0.04 | C |
| rs6446723 | T/C | TC | CC | TC | TT | 0.04 | C |
| rs7685686 | A/G | AG | GG | AG | AA | 0.04 | G |
| rs3733217 | T/C | CC | CC | CC | CC | 0.03 | C |
| rs6844859 | T/C | TC | CC | TC | TT | 0.03 | C |
| rs362331 | T/C | TC | CC | TC | TT | 0.03 | C |

### F. Certain Indications

In certain embodiments, provided herein are methods of treating an animal or individual comprising administering one or more pharmaceutical compositions as described herein. In certain embodiments, the individual or animal has Huntington's disease.

In certain embodiments, compounds targeted to huntingtin as described herein may be administered to reduce the severity of physiological symptoms of Huntington's disease. In certain embodiments, compounds targeted to huntingtin as described herein may be administered to reduce the rate of degeneration in an individual or an animal having Huntington's disease. In certain embodiments, compounds targeted to huntingtin as described herein may be administered regeneration function in an individual or an animal having Huntington's disease. In certain embodiments, symptoms of Huntingtin's disease may be reversed by treatment with a compound as described herein.

In certain embodiments, compounds targeted to huntingtin as described herein may be administered to ameliorate one or more symptoms of Huntington's disease. In certain embodiments administration of compounds targeted to huntingtin as described herein may improve the symptoms of Huntington's disease as measured by any metric known to those having skill in the art. In certain embodiments, administration of compounds targeted to huntingtin as described herein may improve a rodent's rotarod assay performance. In certain embodiments, administration of compounds targeted to huntingtin as described herein may improve a rodent's plus maze assay. In certain embodiments, administration of compounds targeted to huntingtin as described herein may improve a rodent's open field assay performance.

Accordingly, provided herein are methods for ameliorating a symptom associated with Huntington's disease in a subject in need thereof. In certain embodiments, provided is a method for reducing the rate of onset of a symptom associated with Huntington's disease. In certain embodiments, provided is a method for reducing the severity of a symptom associated with Huntington's disease. In certain embodiments, provided is a method for regenerating neurological function as shown by an improvement of a symptom associated with Huntington's disease. In such embodiments, the methods comprise administering to an individual or animal in need thereof a therapeutically effective amount of a compound targeted to a huntingtin nucleic acid.

Huntington's disease is characterized by numerous physical, neurological, psychiatric, and/or peripheral symptoms. Any symptom known to one of skill in the art to be associated with Huntington's disease can be ameliorated or otherwise modulated as set forth above in the methods described above. In certain embodiments, the symptom is a physical symptom selected from the group consisting of restlessness, lack of coordination, unintentionally initiated motions, unintentionally uncompleted motions, unsteady gait, chorea, rigidity, writhing motions, abnormal posturing, instability, abnormal facial expressions, difficulty chewing, difficulty swallowing, difficulty speaking, seizure, and sleep disturbances. In certain embodiments, the symptom is a cognitive symptom selected from the group consisting of impaired planning, impaired flexibility, impaired abstract thinking, impaired rule acquisition, impaired initiation of appropriate actions, impaired inhibition of inappropriate actions, impaired short-term memory, impaired long-term memory, paranoia, disorientation, confusion, hallucination and dementia. In certain embodiments, the symptom is a psychiatric symptom selected from the group consisting of anxiety, depression, blunted affect, egocentrisms, aggression, compulsive behavior, irritability and suicidal ideation. In certain embodiments, the symptom is a peripheral symptom selected from the group consisting of reduced brain mass, muscle atrophy, cardiac failure, impaired glucose tolerance, weight loss, osteoporosis, and testicular atrophy.

In certain embodiments, the symptom is restlessness. In certain embodiments, the symptom is lack of coordination. In certain embodiments, the symptom is unintentionally initiated motions. In certain embodiments, the symptom is unintentionally uncompleted motions. In certain embodiments, the symptom is unsteady gait. In certain embodiments, the symptom is chorea. In certain embodiments, the symptom is rigidity. In certain embodiments, the symptom is writhing motions. In certain embodiments, the symptom is abnormal posturing. In certain embodiments, the symptom is instability. In certain embodiments, the symptom is abnormal facial expressions. In certain embodiments, the symptom is difficulty chewing. In certain embodiments, the symptom is difficulty swallowing. In certain embodiments, the symptom is difficulty speaking. In certain embodiments, the symptom is seizures. In certain embodiments, the symptom is sleep disturbances.

In certain embodiments, the symptom is impaired planning. In certain embodiments, the symptom is impaired flexibility. In certain embodiments, the symptom is impaired abstract thinking. In certain embodiments, the symptom is impaired rule acquisition. In certain embodiments, the symptom is impaired initiation of appropriate actions. In certain embodiments, the symptom is impaired inhibition of inappropriate actions. In certain embodiments, the symptom is impaired short-term memory. In certain embodiments, the symptom is impaired long-term memory. In certain embodiments, the symptom is paranoia. In certain embodiments, the symptom is disorientation. In certain embodiments, the symptom is confusion. In certain embodiments, the symptom is hallucination. In certain embodiments, the symptom is dementia.

In certain embodiments, the symptom is anxiety. In certain embodiments, the symptom is depression. In certain embodiments, the symptom is blunted affect. In certain embodiments, the symptom is egocentrism. In certain embodiments, the symptom is aggression. In certain embodiments, the symptom is compulsive behavior. In certain embodiments, the symptom is irritability. In certain embodiments, the symptom is suicidal ideation.

In certain embodiments, the symptom is reduced brain mass. In certain embodiments, the symptom is muscle atrophy. In certain embodiments, the symptom is cardiac failure. In certain embodiments, the symptom is impaired glucose tolerance. In certain embodiments, the symptom is weight loss. In certain embodiments, the symptom is osteoporosis. In certain embodiments, the symptom is testicular atrophy.

In certain embodiments, symptoms of Huntington's disease may be quantifiable. For example, osteoporosis may be measured and quantified by, for example, bone density scans. For such symptoms, in certain embodiments, the symptom may be reduced by about 15, 20, 25, 30, 35, 40, 45, 50, 55, 60, 65, 70, 75, 80, 85, 90, 95 or 99%, or a range defined by any two of these values.

In certain embodiments, provided are methods of treating an individual comprising administering one or more pharmaceutical compositions as described herein. In certain embodiments, the individual has Huntington's disease.

In certain embodiments, administration of an antisense compound targeted to a huntingtin nucleic acid results in reduction of huntingtin expression by at least about 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense compound targeted to huntingtin are used for the preparation of a medicament for treating a patient suffering or susceptible to Huntington's disease.

### G. Certain Pharmaceutical Compositions

In certain embodiments, the present invention provides pharmaceutical compositions comprising one or more antisense compound. In certain embodiments, such pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one or more antisense compound and sterile water. In certain embodiments, the sterile saline is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile phosphate-buffered saline (PBS). In certain embodiments, the sterile saline is pharmaceutical grade PBS.

In certain embodiments, antisense compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

Pharmaceutical compositions comprising antisense compounds encompass any pharmaceutically acceptable salts, esters, or salts of such esters. In certain embodiments, pharmaceutical compositions comprising antisense compounds comprise one or more oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an oligomeric compound which are cleaved by endogenous nucleases within the body, to form the active antisense oligomeric compound.

Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions provided herein comprise one or more modified oligonucleotides and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, a pharmaceutical composition provided herein comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition provided herein comprises one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, a pharmaceutical composition provided herein comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition provided herein is prepared for oral administration. In certain embodiments, pharmaceutical compositions are prepared for buccal administration.

In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

### H. Administration

In certain embodiments, the compounds and compositions as described herein are administered parenterally.

In certain embodiments, parenteral administration is by infusion. Infusion can be chronic or continuous or short or intermittent. In certain embodiments, infused pharmaceutical agents are delivered with a pump. In certain embodiments, parenteral administration is by injection.

In certain embodiments, compounds and compositions are delivered to the CNS. In certain embodiments, compounds and compositions are delivered to the cerebrospinal fluid. In certain embodiments, compounds and compositions are administered to the brain parenchyma. In certain embodiments, compounds and compositions are delivered to an animal by intrathecal administration, or intracerebroventricular administration. Broad distribution of compounds and compositions, described herein, within the central nervous system may be achieved with intraparenchymal administration, intrathecal administration, or intracerebroventricular administration.

In certain embodiments, parenteral administration is by injection. The injection may be delivered with a syringe or a pump. In certain embodiments, the injection is a bolus injection. In certain embodiments, the injection is administered directly to a tissue, such as striatum, caudate, cortex, hippocampus and cerebellum.

Therefore, in certain embodiments, delivery of a compound or composition described herein can affect the pharmacokinetic profile of the compound or composition. In certain embodiments, injection of a compound or composition described herein, to a targeted tissue improves the pharmacokinetic profile of the compound or composition as compared to infusion of the compound or composition. In a certain embodiment, the injection of a compound or composition improves potency compared to broad diffusion, requiring less of the compound or composition to achieve similar pharmacology. In certain embodiments, similar pharmacology refers to the amount of time that a target mRNA and/or target protein is down-regulated (e.g. duration of action). In certain embodiments, methods of specifically localizing a pharmaceutical agent, such as by bolus injection, decreases median effective concentration (EC50) by a factor of about 50 (e.g. 50 fold less concentration in tissue is required to achieve the same or similar pharmacodynamic effect). In certain embodiments, methods of specifically localizing a pharmaceutical agent, such as by bolus injection, decreases median effective concentration (EC50) by a factor of 20, 25, 30, 35, 40, 45 or 50. In certain embodiments the pharmaceutical agent in an antisense compound as further described herein. In certain embodiments, the targeted tissue is brain tissue. In certain embodiments the targeted tissue is striatal tissue. In certain embodiments, decreasing EC50 is desirable because it reduces the dose required to achieve a pharmacological result in a patient in need thereof.

In certain embodiments, an antisense oligonucleotide is delivered by injection or infusion once every month, every two months, every 90 days, every 3 months, every 6 months, twice a year or once a year.

### I. Certain Combination Therapies

In certain embodiments, one or more pharmaceutical compositions are co-administered with one or more other pharmaceutical agents. In certain embodiments, such one or more other pharmaceutical agents are designed to treat the same disease, disorder, or condition as the one or more pharmaceutical compositions described herein. In certain embodiments, such one or more other pharmaceutical agents are designed to treat a different disease, disorder, or condition as the one or more pharmaceutical compositions described herein. In certain embodiments, such one or more other pharmaceutical agents are designed to treat an undesired side effect of one or more pharmaceutical compositions as described herein. In certain embodiments, one or more pharmaceutical compositions are co-administered with another pharmaceutical agent to treat an undesired effect of that other pharmaceutical agent. In certain embodiments, one or more pharmaceutical compositions are co-administered with another pharmaceutical agent to produce a combinational effect. In certain embodiments, one or more pharmaceutical compositions are co-administered with another pharmaceutical agent to produce a synergistic effect.

In certain embodiments, one or more pharmaceutical compositions and one or more other pharmaceutical agents are administered at the same time. In certain embodiments, one or more pharmaceutical compositions and one or more other pharmaceutical agents are administered at different times. In certain embodiments, one or more pharmaceutical compositions and one or more other pharmaceutical agents are prepared together in a single formulation. In certain embodiments, one or more pharmaceutical compositions and one or more other pharmaceutical agents are prepared separately.

In certain embodiments, pharmaceutical agents that may be co-administered with a pharmaceutical composition of include antipsychotic agents, such as, e.g., haloperidol, chlorpromazine, clozapine, quetapine, and olanzapine; antidepressant agents, such as, e.g., fluoxetine, sertraline hydrochloride, venlafaxine and nortriptyline; tranquilizing agents such as, e.g., benzodiazepines, clonazepam, paroxetine, venlafaxin, and beta-blockers; mood-stabilizing agents such as, e.g., lithium, valproate, lamotrigine, and carbamazepine; paralytic agents such as, e.g., Botulinum toxin; and/or other experimental agents including, but not limited to, tetrabenazine (Xenazine), creatine, conezyme Q10, trehalose, docosahexanoic acids, ACR16, ethyl-EPA, atomoxetine, citalopram, dimebon, memantine, sodium phenylbutyrate, ramelteon, ursodiol, zyprexa, xenasine, tiapride, riluzole, amantadine, [123I]MNI-420, atomoxetine, tetrabenazine, digoxin, detromethorphan, warfarin, alprozam, ketoconazole, omeprazole, and minocycline.

### Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified or naturally occurring bases, such as “AT^{me}CGAUCG,” wherein ^{me}C indicates a cytosine base comprising a methyl group at the 5-position.

### EXAMPLES

The following examples illustrate certain embodiments of the present invention and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

To allow assessment of the relative effects of nucleobase sequence and chemical modification, throughout the examples, oligomeric compounds are assigned a “Sequence Code.” Oligomeric compounds having the same Sequence Code have the same nucleobase sequence. Oligomeric compounds having different Sequence Codes have different nucleobase sequences.

### Example 1: Modified Antisense Oligonucleotides Targeting Human Target-X

Antisense oligonucleotides were designed targeting a Target-X nucleic acid and were tested for their effects on Target-X mRNA in vitro. ISIS 407939, which was described in an earlier publication (WO 2009/061851) was also tested.

The newly designed chimeric antisense oligonucleotides and their motifs are described in Table 15. The internucleoside linkages throughout each gapmer are phosphorothioate linkages (P═S). Nucleosides followed by “d” indicate 2′-deoxyribonucleosides. Nucleosides followed by “k” indicate 6′-(S)—CH₃ bicyclic nucleoside (e.g cEt) nucleosides. Nucleosides followed by “e” indicate 2′-O-methoxyethyl (2′-MOE) nucleosides. “N” indicates modified or naturally occurring nucleobases (A, T, C, G, U, or 5-methyl C).

Each gapmer listed in Table 15 is targeted to the human Target-X genomic sequence.

Activity of the newly designed gapmers was compared to a 5-10-5 2′-MOE gapmer, ISIS 407939 targeting human Target-X and is further described in USPN XXX, incorporated herein by reference. Cultured Hep3B cells at a density of 20,000 cells per well were transfected using electroporation with 2,000 nM antisense oligonucleotide. After a treatment period of approximately 24 hours, RNA was isolated from the cells and Target-X mRNA levels were measured by quantitative real-time PCR. Human primer probe set RTS2927 was used to measure mRNA levels. Target-X mRNA levels were adjusted according to total RNA content, as measured by RIBOGREEN®. Results are presented as percent inhibition of Target-X, relative to untreated control cells, and indicate that several of the newly designed antisense oligonucleotides are more potent than ISIS 407939. A total of 771 oligonucleotides were tested. Only those oligonucleotides which were selected for further studies are shown in Table 15. Each of the newly designed antisense oligonucleotides provided in Table 1 achieved greater than 80% inhibition and, therefore, are more active than ISIS 407939.

TABLE 15
Inhibition of human Target-X mRNA levels by chimeric antisense
oligonucleotides targeted to Target-X

| | | | | | Wing | | | SEQ |
|---|---|---|---|---|---|---|---|---|
| | ISIS | % | | Gap | Chemistry | | SEQ | ID |
| Sequence (5′ to 3′) | NO | inhibition | Motif | Chemistry | 5′ | 3′ | CODE | NO |
| NkNkNkNdNdNdNdNkNd | 473359 | 92 | 3-10-3 | Deoxy/ | kkk | eee | 21 | 6 |
| NdNdNdNdNeNeNe | | | | cEt | | | | |
| NkNkNkNdNdNdNdNkNd | 473360 | 96 | 3-10-3 | Deoxy/ | kkk | eee | 22 | 6 |
| NdNdNdNdNeNeNe | | | | cEt | | | | |
| NkNkNkNdNdNdNdNdNd | 473168 | 94 | 3-10-3 | Full | kkk | kkk | 23 | 6 |
| NdNdNdNdNkNkNk | | | | deoxy | | | | |
| NkNkNkNdNdNdNdNdNd | 473317 | 95 | 3-10-3 | Full | kkk | eee | 23 | 6 |
| NdNdNdNdNeNeNe | | | | deoxy | | | | |
| NkNkNkNdNdNdNdNkNd | 473471 | 90 | 3-10-3 | Deoxy/ | kkk | eee | 23 | 6 |
| NdNdNdNdNeNeNe | | | | cEt | | | | |
| NkNdNkNdNkNdNdNdNd | 473620 | 94 | 5-9-2 | Full | kdkdk | ee | 23 | 6 |
| NdNdNdNdNdNeNe | | | | deoxy | | | | |
| NkNkNdNdNdNdNdNdNd | 473019 | 88 | 2-10-2 | Full | kk | kk | 24 | 7 |
| NdNdNdNkNk | | | | deoxy | | | | |
| NkNkNdNdNdNdNdNdNd | 473020 | 93 | 2-10-2 | Full | kk | kk | 25 | 7 |
| NdNdNdNkNk | | | | deoxy | | | | |
| NkNkNkNdNdNdNdNdNd | 473321 | 93 | 3-10-3 | Full | kkk | eee | 26 | 6 |
| NdNdNdNdNeNeNe | | | | deoxy | | | | |
| NkNkNkNdNdNdNdNdNd | 473322 | 94 | 3-10-3 | Full | kkk | eee | 27 | 6 |
| NdNdNdNdNeNeNe | | | | deoxy | | | | |
| NkNkNkNdNdNdNdNdNd | 473323 | 96 | 3-10-3 | Full | kkk | eee | 28 | 6 |
| NdNdNdNdNeNeNe | | | | deoxy | | | | |
| NkNkNkNdNdNdNdNdNd | 473326 | 94 | 3-10-3 | Full | kkk | eee | 29 | 6 |
| NdNdNdNdNeNeNe | | | | deoxy | | | | |
| NkNkNkNdNdNdNdNkNd | 473480 | 92 | 3-10-3 | Deoxy/ | kkk | eee | 29 | 6 |
| NdNdNdNdNeNeNe | | | | cEt | | | | |
| NkNkNkNdNdNdNdNdNd | 473178 | 96 | 3-10-3 | Full | kkk | kkk | 30 | 6 |
| NdNdNdNdNkNkNk | | | | deoxy | | | | |
| NkNkNkNdNdNdNdNdNd | 473327 | 96 | 3-10-3 | Full | kkk | eee | 30 | 6 |
| NdNdNdNdNeNeNe | | | | deoxy | | | | |
| NkNkNkNdNdNdNdNkNd | 473481 | 93 | 3-10-3 | Deoxy/ | kkk | eee | 30 | 6 |
| NdNdNdNdNeNeNe | | | | cEt | | | | |
| NkNdNkNdNkNdNdNdNd | 473630 | 89 | 5-9-2 | Full | kdkdk | ee | 30 | 6 |
| NdNdNdNdNdNeNe | | | | deoxy | | | | |
| NkNkNdNdNdNdNdNdNd | 473029 | 96 | 2-10-2 | Full | kk | kk | 31 | 7 |
| NdNdNdNkNk | | | | deoxy | | | | |
| NkNkNdNdNdNdNdNdNd | 472925 | 93 | 2-10-2 | Full | kk | kk | 32 | 7 |
| NdNdNdNkNk | | | | deoxy | | | | |
| NkNkNdNdNdNdNdNdNd | 472926 | 85 | 2-10-2 | Full | kk | kk | 33 | 7 |
| NdNdNdNkNk | | | | deoxy | | | | |
| NkNkNkNdNdNdNdNdNd | 473195 | 97 | 3-10-3 | Full | kkk | kkk | 34 | 6 |
| NdNdNdNdNkNkNk | | | | deoxy | | | | |
| NkNkNdNdNdNdNdNdNd | 473046 | 90 | 2-10-2 | Full | kk | kk | 35 | 7 |
| NdNdNdNkNk | | | | deoxy | | | | |
| NkNkNdNdNdNdNdNdNd | 472935 | 92 | 2-10-2 | Full | kk | kk | 36 | 7 |
| NdNdNdNkNk | | | | deoxy | | | | |
| NkNkNkNdNdNdNdNdNd | 473089 | 95 | 3-10-3 | Full | kkk | kkk | 37 | 6 |
| NdNdNdNdNkNkNk | | | | deoxy | | | | |
| NkNkNkNdNdNdNdNdNd | 473350 | 93 | 3-10-3 | Full | kkk | eee | 38 | 6 |
| NdNdNdNdNeNeNe | | | | deoxy | | | | |
| NkNkNkNdNdNdNdNdNd | 473353 | 93 | 3-10-3 | Full | kkk | eee | 39 | 6 |
| NdNdNdNdNeNeNe | | | | deoxy | | | | |
| NkNkNdNdNdNdNdNdNd | 473055 | 91 | 2-10-2 | Full | kk | kk | 40 | 7 |
| NdNdNdNkNk | | | | deoxy | | | | |
| NkNkNkNdNdNdNdNkNd | 473392 | 95 | 3-10-3 | Deoxy/ | kkk | eee | 41 | 6 |
| NdNdNdNdNeNeNe | | | | cEt | | | | |
| NkNkNkNdNdNdNdNdNd | 473095 | 100 | 3-10-3 | Full | kkk | kkk | 42 | 6 |
| NdNdNdNdNkNkNk | | | | deoxy | | | | |
| NkNkNkNdNdNdNdNdNd | 473244 | 99 | 3-10-3 | Full | kkk | eee | 42 | 6 |
| NdNdNdNdNeNeNe | | | | deoxy | | | | |
| NkNkNkNdNdNdNdNkNd | 473393 | 99 | 3-10-3 | Deoxy/ | kkk | eee | 42 | 6 |
| NdNdNdNdNeNeNe | | | | cEt | | | | |
| NkNdNkNdNkNdNdNdNd | 473547 | 98 | 5-9-2 | Full | kdkdk | ee | 42 | 6 |
| NdNdNdNdNdNeNe | | | | deoxy | | | | |
| NkNkNdNdNdNdNdNdNd | 472942 | 87 | 2-10-2 | Full | kk | kk | 43 | 7 |
| NdNdNdNkNk | | | | deoxy | | | | |
| NkNkNkNdNdNdNdNdNd | 473098 | 97 | 3-10-3 | Full | kkk | kkk | 44 | 6 |
| NdNdNdNdNkNkNk | | | | deoxy | | | | |
| NkNkNkNdNdNdNdNkNd | 473408 | 92 | 3-10-3 | Deoxy/ | kkk | eee | 45 | 6 |
| NdNdNdNdNeNeNe | | | | cEt | | | | |
| NkNkNdNdNdNdNdNdNd | 472958 | 89 | 2-10-2 | Full | kk | kk | 46 | 7 |
| NdNdNdNkNk | | | | deoxy | | | | |
| NkNkNdNdNdNdNdNdNd | 472959 | 90 | 2-10-2 | Full | kk | kk | 47 | 7 |
| NdNdNdNkNk | | | | deoxy | | | | |
| NkNdNkNdNkNdNdNdNd | 473566 | 94 | 5-9-2 | Full | kdkdk | ee | 48 | 6 |
| NdNdNdNdNdNeNe | | | | deoxy | | | | |
| NkNdNkNdNkNdNdNdNd | 473567 | 95 | 5-9-2 | Full | kdkdk | ee | 49 | 6 |
| NdNdNdNdNdNeNe | | | | deoxy | | | | |
| NkNdNkNdNkNdNdNdNd | 473569 | 92 | 5-9-2 | Full | kdkdk | ee | 50 | 6 |
| NdNdNdNdNdNeNe | | | | deoxy | | | | |
| NkNkNdNdNdNdNdNdNd | 457851 | 90 | 2-10-2 | Full | kk | kk | 51 | 7 |
| NdNdNdNkNk | | | | deoxy | | | | |
| NkNkNdNdNdNdNdNdNd | 472970 | 91 | 2-10-2 | Full | kk | kk | 32 | 7 |
| NdNdNdNkNk | | | | deoxy | | | | |
| NkNkNkNdNdNdNdNdNd | 473125 | 90 | 3-10-3 | Full | kkk | kkk | 53 | 6 |
| NdNdNdNdNkNkNk | | | | deoxy | | | | |
| NkNkNkNdNdNdNdNdNd | 473274 | 98 | 3-10-3 | Full | kkk | eee | 53 | 6 |
| NdNdNdNdNeNeNe | | | | deoxy | | | | |
| NkNkNkNdNdNdNdNkNd | 473428 | 90 | 3-10-3 | Deoxy/ | kkk | eee | 53 | 6 |
| NdNdNdNdNeNeNe | | | | cEt | | | | |
| NkNdNkNdNkNdNdNdNd | 473577 | 93 | 5-9-2 | Full | kdkdk | ee | 53 | 6 |
| NdNdNdNdNdNeNe | | | | deoxy | | | | |
| NkNkNdNdNdNdNdNdNd | 472976 | 97 | 2-10-2 | Full | kk | kk | 54 | 7 |
| NdNdNdNkNk | | | | deoxy | | | | |
| NkNkNdNdNdNdNdNd | 472983 | 94 | 2-10-2 | Full | kk | kk | 55 | 7 |
| NdNdNdNdNkNk | | | | deoxy | | | | |
| NkNkNdNdNdNdNdNd | 472984 | 90 | 2-10-2 | Full | kk | kk | 56 | 7 |
| NdNdNdNdNkNk | | | | deoxy | | | | |
| NkNkNkNdNdNdNdNd | 473135 | 97 | 3-10-3 | Full | kkk | kkk | 57 | 6 |
| NdNdNdNdNdNkNkNk | | | | deoxy | | | | |
| NkNkNdNdNdNdNdNd | 472986 | 95 | 2-10-2 | Full | kk | kk | 58 | 7 |
| NdNdNdNdNkNk | | | | deoxy | | | | |
| NkNkNkNdNdNdNdNd | 473137 | 95 | 3-10-3 | Full | kkk | kkk | 59 | 6 |
| NdNdNdNdNdNkNkNk | | | | deoxy | | | | |
| NkNkNkNdNdNdNdNd | 473286 | 95 | 3-10-3 | Full | kkk | eee | 59 | 6 |
| NdNdNdNdNdNeNeNe | | | | deoxy | | | | |
| NkNkNkNdNdNdNdNkNd | 473440 | 88 | 3-10-3 | Deoxy/ | kkk | eee | 59 | 6 |
| NdNdNdNdNeNeNe | | | | cEt | | | | |
| NkNdNkNdNkNdNdNd | 473589 | 97 | 5-9-2 | Full | kdkdk | ee | 59 | 6 |
| NdNdNdNdNdNdNeNe | | | | deoxy | | | | |
| NkNkNdNdNdNdNdNd | 472988 | 85 | 2-10-2 | Full | kk | kk | 60 | 7 |
| NdNdNdNdNkNk | | | | deoxy | | | | |
| NkNkNkNdNdNdNdNd | 473140 | 96 | 3-10-3 | Full | kkk | kkk | 61 | 6 |
| NdNdNdNdNdNkNkNk | | | | deoxy | | | | |
| NkNkNdNdNdNdNdNd | 472991 | 90 | 2-10-2 | Full | kk | kk | 62 | 7 |
| NdNdNdNdNkNk | | | | deoxy | | | | |
| NkNkNkNdNdNdNdNkNd | 473444 | 94 | 3-10-3 | Deoxy/ | kkk | eee | 63 | 6 |
| NdNdNdNdNeNeNe | | | | cEt | | | | |
| NkNkNkNdNdNdNdNd | 473142 | 96 | 3-10-3 | Full | kkk | kkk | 64 | 6 |
| NdNdNdNdNdNkNkNk | | | | deoxy | | | | |
| NkNkNkNdNdNdNdNd | 473291 | 95 | 3-10-3 | Full | kkk | eee | 64 | 6 |
| NdNdNdNdNdNeNeNe | | | | deoxy | | | | |
| NkNdNkNdNkNdNdNd | 473594 | 95 | 5-9-2 | Full | kdkdk | ee | 64 | 6 |
| NdNdNdNdNdNdNeNe | | | | deoxy | | | | |
| NkNkNkNdNdNdNdNdNd | 473143 | 97 | 3-10-3 | Full | kkk | kkk | 65 | 6 |
| NdNdNdNdNkNkNk | | | | deoxy | | | | |
| NkNkNkNdNdNdNdNd | 473292 | 96 | 3-10-3 | Full | kkk | eee | 65 | 6 |
| NdNdNdNdNdNeNeNe | | | | deoxy | | | | |
| NkNkNkNdNdNdNdNkNd | 473446 | 96 | 3-10-3 | Deoxy/ | kkk | eee | 65 | 6 |
| NdNdNdNdNeNeNe | | | | cEt | | | | |
| NkNdNkNdNkNdNdNdNd | 473595 | 84 | 5-9-2 | Full | kdkdk | ee | 65 | 6 |
| NdNdNdNdNdNeNe | | | | deoxy | | | | |
| NkNkNdNdNdNdNdNdNd | 472994 | 96 | 2-10-2 | Full | kk | kk | 66 | 7 |
| NdNdNdNkNk | | | | deoxy | | | | |
| NkNkNkNdNdNdNdNdNd | 473144 | 98 | 3-10-3 | Full | kkk | kkk | 67 | 6 |
| NdNdNdNdNkNkNk | | | | deoxy | | | | |
| NkNkNkNdNdNdNdNdNd | 473293 | 96 | 3-10-3 | Full | kkk | eee | 67 | 6 |
| NdNdNdNdNeNeNe | | | | deoxy | | | | |
| NkNkNdNdNdNdNdNdNd | 472995 | 96 | 2-10-2 | Full | kk | kk | 68 | 7 |
| NdNdNdNkNk | | | | deoxy | | | | |
| NkNkNkNdNdNdNdNd | 473294 | 91 | 3-10-3 | Full | kkk | eee | 69 | 6 |
| NdNdNdNdNdNeNeNe | | | | deoxy | | | | |
| NkNdNkNdNkNdNdNdNd | 473597 | 94 | 5-9-2 | Full | kdkdk | ee | 69 | 6 |
| NdNdNdNdNdNeNe | | | | deoxy | | | | |
| NkNkNdNdNdNdNdNdNd | 472996 | 94 | 2-10-2 | Full | kk | kk | 70 | 7 |
| NdNdNdNkNk | | | | deoxy | | | | |
| NkNkNkNdNdNdNdNd | 473295 | 92 | 3-10-3 | Full | kkk | eee | 71 | 6 |
| NdNdNdNdNdNeNeNe | | | | deoxy | | | | |
| NeNeNeNeNeNdNdNdNdNd | 407939 | 80 | 5-10-5 | Full | eeeee | eeee | 72 | 8 |
| NdNdNdNdNdNeNeNeNeNe | | | | deoxy | | e | | |
| NkNkNkNdNdNdNdNdNd | 473296 | 98 | 3-10-3 | Full | kkk | eee | 73 | 6 |
| NdNdNdNdNeNeNe | | | | deoxy | | | | |
| NkNkNkNdNdNdNdNkNd | 473450 | 95 | 3-10-3 | Deoxy/ | kkk | eee | 73 | 6 |
| NdNdNdNdNeNeNe | | | | cEt | | | | |
| NkNkNdNdNdNdNdNdNd | 472998 | 97 | 2-10-2 | Full | kk | kk | 74 | 7 |
| NdNdNdNkNk | | | | deoxy | | | | |

e = 2′MOE, k = cEt, d = 2′-deoxyribonucleoside

### Example 2: Modified Antisense Oligonucleotides Comprising 6′-(S)—CH₃ Bicyclic Nucleoside (cEt) and F-HNA Modifications Targeting Human Target-X

Additional antisense oligonucleotides were designed targeting a Target-X nucleic acid and were tested for their effects on Target-X mRNA in vitro. ISIS 407939 was also tested.

The chimeric antisense oligonucleotides and their motifs are described in Table 16. The internucleoside linkages throughout each gapmer are phosphorothioate linkages (P═S). Nucleosides followed by “d” indicate 2′-deoxyribonucleosides. Nucleosides followed by “k” indicate 6′-(S)—CH₃ bicyclic nucleosides (e.g cEt). Nucleosides followed by “e” indicate 2′-O-methoxyethyl (2′-MOE) modified nucleosides. Nucleosides followed by ‘g’ indicate F-HNA modified nucleosides. “N” indicates modified or naturally occurring nucleobases (A, T, C, G, U, or 5-methyl C).

Each gapmer listed in Table 16 is targeted to the human Target-X genomic sequence.

Activity of the newly designed gapmers was compared to a 5-10-5 2′-MOE gapmer, ISIS 407939 targeting human Target-X. Cultured Hep3B cells at a density of 20,000 cells per well were transfected using electroporation with 2,000 nM antisense oligonucleotide. After a treatment period of approximately 24 hours, RNA was isolated from the cells and Target-X mRNA levels were measured by quantitative real-time PCR. Human primer probe set RTS2927 was used to measure mRNA levels. Target-X mRNA levels were adjusted according to total RNA content, as measured by RIBOGREEN®. Results are presented as percent inhibition of Target-X, relative to untreated control cells, and demonstrate that several of the newly designed gapmers are more potent than ISIS 407939. A total of 765 oligonucleotides were tested. Only those oligonucleotides which were selected for further studies are shown in Table 16. All but one of the newly designed antisense oligonucleotides provided in Table 16 achieved greater than 3000 inhibition and, therefore, are more active than ISIS 407939.

TABLE 16
Inhibition of human Target-X mRNA levels by chimeric antisense
oligonucleotides targeted to Target-X

| | | | | | Wing | | | SEQ |
|---|---|---|---|---|---|---|---|---|
| | ISIS | % | | Gap | Chemistry | | SEQ | ID |
| Sequence (5′ to 3′) | No | inhibition | Motif | Chemistry | 5′ | 3′ | CODE | NO |
| NgNgNdNdNdNdNdNdNd | 482838 | 81 | 2-10-2 | Full | gg | gg | 25 | 7 |
| NdNdNdNgNg | | | | deoxy | | | | |
| NgNgNgNdNdNdNdNdNd | 482992 | 93 | 3-10-3 | Full | ggg | ggg | 28 | 6 |
| NdNdNdNdNgNgNg | | | | deoxy | | | | |
| NgNgNgNdNdNdNdNdNd | 482996 | 97 | 3-10-3 | Full | ggg | ggg | 30 | 6 |
| NdNdNdNdNgNgNg | | | | deoxy | | | | |
| NgNdNgNdNgNdNdNdNd | 483284 | 82 | 5-9-2 | Full | gdgdg | ee | 23 | 6 |
| NdNdNdNdNdNeNe | | | | deoxy | | | | |
| NgNdNgNdNgNdNdNdNd | 483289 | 70 | 5-9-2 | Full | gdgdg | ee | 27 | 6 |
| NdNdNdNdNdNeNe | | | | deoxy | | | | |
| NgNdNgNdNgNdNdNdNd | 483290 | 80 | 5-9-2 | Full | gdgdg | ee | 28 | 6 |
| NdNdNdNdNdNeNe | | | | deoxy | | | | |
| NgNdNgNdNgNdNdNdNd | 483294 | 69 | 5-9-2 | Full | gdgdg | ee | 30 | 6 |
| NdNdNdNdNdNeNe | | | | deoxy | | | | |
| NgNgNdNdNdNdNdNdNd | 483438 | 81 | 2-10-4 | Full | gg | eeee | 23 | 6 |
| NdNdNdNeNeNeNe | | | | deoxy | | | | |
| NgNgNdNdNdNdNdNdNd | 483444 | 84 | 2-10-4 | Full | gg | eeee | 28 | 6 |
| NdNdNdNeNeNeNe | | | | deoxy | | | | |
| NgNgNdNdNdNdNdNdNd | 483448 | 77 | 2-10-4 | Full | gg | eeee | 30 | 6 |
| NdNdNdNeNeNeNe | | | | deoxy | | | | |
| NgNgNdNdNdNdNdNdNd | 482847 | 79 | 2-10-2 | Full | gg | gg | 31 | 7 |
| NdNdNdNgNg | | | | deoxy | | | | |
| NgNgNdNdNdNdNdNdNd | 482747 | 85 | 2-10-2 | Full | gg | gg | 32 | 7 |
| NdNdNdNgNg | | | | deoxy | | | | |
| NgNgNdNdNdNdNdNdNd | 482873 | 81 | 2-10-2 | Full | gg | gg | 40 | 7 |
| NdNdNdNgNg | | | | deoxy | | | | |
| NgNgNdNdNdNdNdNdNdNd | 482874 | 82 | 2-10-2 | Full | gg | gg | 75 | 7 |
| NdNdNgNg | | | | deoxy | | | | |
| NgNgNdNdNdNdNdNd | 482875 | 82 | 2-10-2 | Full | gg | gg | 76 | 7 |
| NdNdNdNdNgNg | | | | deoxy | | | | |
| NgNgNgNdNdNdNdNd | 482896 | 95 | 3-10-3 | Full | ggg | ggg | 77 | 6 |
| NdNdNdNdNdNgNgNg | | | | deoxy | | | | |
| NgNgNgNdNdNdNdNdNd | 483019 | 89 | 3-10-3 | Full | ggg | ggg | 38 | 6 |
| NdNdNdNdNgNgNg | | | | deoxy | | | | |
| NgNdNgNdNdNdNdNdNd | 483045 | 92 | 3-10-3 | Full | gdg | gdg | 77 | 6 |
| NdNdNdNdNgNdNg | | | | deoxy | | | | |
| NgNdNgNdNgNdNdNdNd | 483194 | 64 | 3-10-3 | Full | gdg | gdg | 77 | 6 |
| NdNdNdNdNdNeNe | | | | deoxy | | | | |
| NgNdNgNdNgNdNdNdNd | 483317 | 79 | 5-9-2 | Full | gdgdg | ee | 38 | 6 |
| NdNdNdNdNdNeNe | | | | deoxy | | | | |
| NgNgNdNdNdNdNdNdNd | 483343 | 75 | 2-10-4 | Full | gg | eeee | 57 | 6 |
| NdNdNdNeNeNeNe | | | | deoxy | | | | |
| NgNgNdNdNdNdNdNdNdNdN | 483471 | 76 | 2-10-4 | Full | gg | eeee | 38 | 6 |
| dNdNeNeNeNe | | | | deoxy | | | | |
| NgNgNdNdNdNdNdNdNd | 483478 | 20 | 2-10-4 | Full | gg | eeee | 78 | 6 |
| NdNdNdNeNeNeNe | | | | deoxy | | | | |
| NeNeNeNeNeNdNdNdNdNd | 407939 | 30 | 5-10-5 | Full | eeeee | eeeee | 72 | 8 |
| NdNdNdNdNdNeNeNeNeNe | | | | deoxy | | | | |
| NgNgNdNdNdNdNdNd | 482784 | 83 | 2-10-2 | Full | gg | gg | 79 | 7 |
| NdNdNdNdNgNg | | | | deoxy | | | | |
| NgNgNdNdNdNdNdNd | 482794 | 91 | 2-10-2 | Full | gg | gg | 54 | 7 |
| NdNdNdNdNgNg | | | | deoxy | | | | |
| NgNgNdNdNdNdNdNd | 482804 | 80 | 2-10-2 | Full | gg | gg | 58 | 7 |
| NdNdNdNdNgNg | | | | deoxy | | | | |
| NgNgNdNdNdNdNdNd | 482812 | 81 | 2-10-2 | Full | gg | gg | 66 | 7 |
| NdNdNdNdNgNg | | | | deoxy | | | | |
| NgNgNdNdNdNdNdNd | 482813 | 92 | 2-10-2 | Full | gg | gg | 68 | 7 |
| NdNdNdNdNgNg | | | | deoxy | | | | |
| NgNgNdNdNdNdNdNd | 482814 | 94 | 2-10-2 | Full | gg | gg | 70 | 7 |
| NdNdNdNdNgNg | | | | deoxy | | | | |
| NgNgNdNdNdNdNdNd | 482815 | 81 | 2-10-2 | Full | gg | gg | 80 | 7 |
| NdNdNdNdNgNg | | | | deoxy | | | | |
| NgNgNdNdNdNdNdNd | 482816 | 71 | 2-10-2 | Full | gg | gg | 74 | 7 |
| NdNdNdNdNgNg | | | | deoxy | | | | |
| NgNgNgNdNdNdNdNd | 482916 | 90 | 3-10-3 | Full | ggg | ggg | 44 | 6 |
| NdNdNdNdNdNgNgNg | | | | deoxy | | | | |
| NgNgNgNdNdNdNdNd | 482932 | 89 | 3-10-3 | Full | ggg | ggg | 48 | 6 |
| NdNdNdNdNdNgNgNg | | | | deoxy | | | | |
| NgNgNgNdNdNdNdNd | 482953 | 93 | 3-10-3 | Full | ggg | ggg | 57 | 6 |
| NdNdNdNdNdNgNgNg | | | | deoxy | | | | |
| NgNgNgNdNdNdNdNd | 482962 | 97 | 3-10-3 | Full | ggg | ggg | 67 | 6 |
| NdNdNdNdNdNgNgNg | | | | deoxy | | | | |
| NgNgNgNdNdNdNdNd | 482963 | 96 | 3-10-3 | Full | ggg | ggg | 69 | 6 |
| NdNdNdNdNdNgNgNg | | | | deoxy | | | | |
| NgNgNgNdNdNdNdNd | 482965 | 89 | 3-10-3 | Full | ggg | ggg | 73 | 6 |
| NdNdNdNdNdNgNgNg | | | | deoxy | | | | |
| NgNdNgNdNdNdNdNd | 483065 | 69 | 3-10-3 | Full | ggg | ggg | 44 | 6 |
| NdNdNdNdNdNgNdNg | | | | deoxy | | | | |
| NgNdNgNdNdNdNdNd | 483092 | 89 | 3-10-3 | Full | gdg | gdg | 53 | 6 |
| NdNdNdNdNdNgNdNg | | | | deoxy | | | | |
| NgNdNgNdNgNdNdNd | 483241 | 79 | 5-9-2 | Full | gdgdg | ee | 53 | 6 |
| NdNdNdNdNdNdNeNe | | | | deoxy | | | | |
| NgNdNgNdNgNdNdNd | 483253 | 76 | 5-9-2 | Full | gdgdg | ee | 59 | 6 |
| NdNdNdNdNdNdNeNe | | | | deoxy | | | | |
| NgNdNgNdNgNdNdNd | 483258 | 70 | 5-9-2 | Full | gdgdg | ee | 64 | 6 |
| NdNdNdNdNdNdNeNe | | | | deoxy | | | | |
| NgNdNgNdNgNdNdNd | 483260 | 62 | 5-9-2 | Full | gdgdg | ee | 67 | 6 |
| NdNdNdNdNdNdNeNe | | | | deoxy | | | | |
| NgNdNgNdNgNdNdNd | 483261 | 76 | 5-9-2 | Full | gdgdg | ee | 69 | 6 |
| NdNdNdNdNdNdNeNe | | | | deoxy | | | | |
| NgNdNgNdNgNdNdNd | 483262 | 75 | 5-9-2 | Full | gdgdg | ee | 71 | 6 |
| NdNdNdNdNdNdNeNe | | | | deoxy | | | | |
| NgNdNgNdNgNdNdNd | 483263 | 73 | 5-9-2 | Full | gdgdg | ee | 73 | 6 |
| NdNdNdNdNdNdNeNe | | | | deoxy | | | | |
| NgNgNdNdNdNdNdNd | 483364 | 78 | 2-10-4 | Full | gg | eeee | 81 | 6 |
| NdNdNdNdNeNeNeNe | | | | deoxy | | | | |
| NgNgNdNdNdNdNdNd | 483395 | 86 | 2-10-4 | Full | gg | eeee | 53 | 6 |
| NdNdNdNdNeNeNeNe | | | | deoxy | | | | |
| NgNgNdNdNdNdNdNd | 483413 | 83 | 2-10-4 | Full | gg | eeee | 65 | 6 |
| NdNdNdNdNeNeNeNe | | | | deoxy | | | | |
| NgNgNdNdNdNdNdNd | 483414 | 76 | 2-10-4 | Full | gg | eeee | 67 | 6 |
| NdNdNdNdNeNeNeNe | | | | deoxy | | | | |
| NgNgNdNdNdNdNdNd | 483415 | 85 | 2-10-4 | Full | gg | eeee | 69 | 6 |
| NdNdNdNdNeNeNeNe | | | | deoxy | | | | |
| NgNgNdNdNdNdNdNd | 483416 | 77 | 2-10-4 | Full | gg | eeee | 71 | 6 |
| NdNdNdNdNeNeNeNe | | | | deoxy | | | | |
| NgNgNdNdNdNdNdNd | 483417 | 83 | 2-10-4 | Full | gg | eeee | 73 | 6 |
| NdNdNdNdNeNeNeNe | | | | deoxy | | | | |

e = 2′-MOE, d = 2′-deoxyribonucleoside, g = F-HNA

### Example 3: Modified Antisense Oligonucleotides Comprising 2′-MOE and 6′-(S)—CH₃ Bicyclic Nucleoside (e.g cEt) Modifications Targeting Human Target-X

Additional antisense oligonucleotides were designed targeting a Target-X nucleic acid and were tested for their effects on Target-X mRNA in vitro. ISIS 403052, ISIS 407594, ISIS 407606, ISIS 407939, and ISIS 416438, which were described in an earlier publication (WO 2009/061851) were also tested.

The newly designed chimeric antisense oligonucleotides are 16 nucleotides in length and their motifs are described in Table 17. The chemistry column of Table 17 presents the sugar motif of each oligonucleotide, wherein “e” indicates a 2′-O-methoxyethyl (2′-MOE) nucleoside, “k” indicates a 6′-(S)—CH₃ bicyclic nucleoside (e.g cEt) and “d” indicates a 2′-deoxyribonucleoside. The internucleoside linkages throughout each gapmer are phosphorothioate (P═S) linkages. All cytosine residues throughout each oligonucleotide are 5-methylcytosines.

Each gapmer listed in Table 17 is targeted to the human Target-X genomic sequence.

Activity of the newly designed gapmers was compared to ISIS 403052, ISIS 407594, ISIS 407606, ISIS 407939, and ISIS 416438. Cultured Hep3B cells at a density of 20,000 cells per well were transfected using electroporation with 2,000 nM antisense oligonucleotide. After a treatment period of approximately 24 hours, RNA was isolated from the cells and Target-X mRNA levels were measured by quantitative real-time PCR. Human primer probe set RTS2927 (described hereinabove in Example 1) was used to measure mRNA levels. Target-X mRNA levels were adjusted according to total RNA content, as measured by RIBOGREEN. Results are presented as percent inhibition of Target-X, relative to untreated control cells. A total of 380 oligonucleotides were tested. Only those oligonucleotides which were selected for further studies are shown in Table 17. Each of the newly designed antisense oligonucleotides provided in Table 17 achieved greater than 64% inhibition and, therefore, are more potent than each of ISIS 403052, ISIS 407594, ISIS 407606, ISIS 407939, and ISIS 416438.

TABLE 17
Inhibition of human Target-X mRNA levels by chimeric
antisense oligonucleotides targeted to Target-X

| ISIS No | Chemistry | Motif | % inhibition | SEQ CODE |
|---|---|---|---|---|
| 403052 | eeeee-(d10)-eeeee | 5-10-5 | 64 | 82 |
| 407594 | eeeee-(d10)-eeeee | 5-10-5 | 40 | 83 |
| 407606 | eeeee-(d10)-eeeee | 5-10-5 | 39 | 84 |
| 407939 | eeeee-(d10)-eeeee | 5-10-5 | 57 | 72 |
| 416438 | eeeee-(d10)-eeeee | 5-10-5 | 62 | 85 |
| 484487 | kdk-(d10)-dkdk | 3-10-3 | 91 | 77 |
| 484539 | kdk-d(10)-kdk | 3-10-3 | 92 | 53 |
| 484546 | kdk-d(10)-kdk | 3-10-3 | 92 | 86 |
| 484547 | kdk-d(10)-kdk | 3-10-3 | 89 | 87 |
| 484549 | kdk-d(10)-kdk | 3-10-3 | 91 | 57 |
| 484557 | kdk-d(10)-kdk | 3-10-3 | 92 | 65 |
| 484558 | kdk-d(10)-kdk | 3-10-3 | 94 | 67 |
| 484559 | kdk-d(10)-kdk | 3-10-3 | 90 | 69 |
| 484582 | kdk-d(10)-kdk | 3-10-3 | 88 | 23 |
| 484632 | kk-d(10)-eeee | 2-10-4 | 90 | 88 |
| 484641 | kk-d(10)-eeee | 2-10-4 | 91 | 77 |
| 484679 | kk-d(10)-eeee | 2-10-4 | 90 | 49 |
| 484693 | kk-d(10)-eeee | 2-10-4 | 93 | 53 |
| 484711 | kk-d(10)-eeee | 2-10-4 | 92 | 65 |
| 484712 | kk-d(10)-eeee | 2-10-4 | 92 | 67 |
| 484713 | kk-d(10)-eeee | 2-10-4 | 85 | 69 |
| 484714 | kk-d(10)-eeee | 2-10-4 | 83 | 71 |
| 484715 | kk-d(10)-eeee | 2-10-4 | 93 | 73 |
| 484736 | kk-d(10)-eeee | 2-10-4 | 89 | 23 |
| 484742 | kk-d(10)-eeee | 2-10-4 | 93 | 28 |
| 484746 | kk-d(10)-eeee | 2-10-4 | 88 | 30 |
| 484771 | kk-d(10)-eeee | 2-10-4 | 89 | 89 |

e = 2′-MOE, k = cEt, d = 2′-deoxyribonucleoside

### Example 4: Antisense Inhibition of Human Target-X with 5-10-5 2′-MOE Gapmers

Additional antisense oligonucleotides were designed targeting a Target-X nucleic acid and were tested for their effects on Target-X mRNA in vitro. Also tested were ISIS 403094, ISIS 407641, ISIS 407643, ISIS 407662, ISIS 407900, ISIS 407910, ISIS 407935, ISIS 407936, ISIS 407939, ISIS 416446, ISIS 416449, ISIS 416455, ISIS 416472, ISIS 416477, ISIS 416507, ISIS 416508, ISIS 422086, ISIS 422087, ISIS 422140, and ISIS 422142, 5-10-5 2′-MOE gapmers targeting human Target-X, which were described in an earlier publication (WO 2009/061851), incorporated herein by reference.

The newly designed modified antisense oligonucleotides are 20 nucleotides in length and their motifs are described in Tables 18 and 19. The chemistry column of Tables 18 and 19 present the sugar motif of each oligonucleotide, wherein “e” indicates a 2′-O-methoxyethyl (2′-MOE) nucleoside and “d” indicates a 2′-deoxyribonucleoside. The internucleoside linkages throughout each gapmer are phosphorothioate (P═S) linkages. All cytosine residues throughout each oligonucleotide are 5-methylcytosines.

Each gapmer listed in Table 18 is targeted to the human Target-X genomic sequence.

Activity of the newly designed gapmers was compared to ISIS 403094, ISIS 407641, ISIS 407643, ISIS 407662, ISIS 407900, ISIS 407910, ISIS 407935, ISIS 407936, ISIS 407939, ISIS 416446, ISIS 416449, ISIS 416455, ISIS 416472, ISIS 416477, ISIS 416507, ISIS 416508, ISIS 422086, ISIS 422087, ISIS 422140, and ISIS 422142. Cultured Hep3B cells at a density of 20,000 cells per well were transfected using electroporation with 2,000 nM antisense oligonucleotide. After a treatment period of approximately 24 hours, RNA was isolated from the cells and Target-X mRNA levels were measured by quantitative real-time PCR. Human primer probe set RTS2927 (described hereinabove in Example 1) was used to measure mRNA levels. Target-X mRNA levels were adjusted according to total RNA content, as measured by RIBOGREEN. Results are presented as percent inhibition of Target-X, relative to untreated control cells. A total of 916 oligonucleotides were tested. Only those oligonucleotides which were selected for further studies are shown in Tables 18 and 19.

TABLE 18
Inhibition of human Target-X mRNA levels by chimeric
antisense oligonucleotides targeted to Target-X

| ISIS No | Chemistry | % inhibition | SEQ CODE |
|---|---|---|---|
| 490275 | e5-d(10)-e5 | 35 | 90 |
| 490277 | e5-d(10)-e5 | 73 | 91 |
| 490278 | e5-d(10)-e5 | 78 | 92 |
| 490279 | e5-d(10)-e5 | 66 | 93 |
| 490323 | e5-d(10)-e5 | 65 | 94 |
| 490368 | e5-d(10)-e5 | 78 | 95 |
| 490396 | e5-d(10)-e5 | 76 | 96 |
| 416507 | e5-d(10)-e5 | 73 | 97 |
| 422140 | e5-d(10)-e5 | 59 | 98 |
| 422142 | e5-d(10)-e5 | 73 | 99 |
| 416508 | e5-d(10)-e5 | 75 | 100 |
| 490424 | e5-d(10)-e5 | 57 | 101 |
| 490803 | e5-d(10)-e5 | 70 | 102 |
| 416446 | e5-d(10)-e5 | 73 | 103 |
| 416449 | e5-d(10)-e5 | 33 | 104 |
| 407900 | e5-d(10)-e5 | 66 | 105 |
| 490103 | e5-d(10)-e5 | 87 | 106 |
| 416455 | e5-d(10)-e5 | 42 | 107 |
| 407910 | e5-d(10)-e5 | 25 | 108 |
| 490149 | e5-d(10)-e5 | 82 | 109 |
| 403094 | e5-d(10)-e5 | 60 | 110 |
| 416472 | e5-d(10)-e5 | 78 | 111 |
| 407641 | e5-d(10)-e5 | 64 | 112 |
| 416477 | e5-d(10)-e5 | 25 | 113 |
| 407643 | e5-d(10)-e5 | 78 | 114 |
| 490196 | e5-d(10)-e5 | 81 | 115 |
| 490197 | e5-d(10)-e5 | 85 | 116 |
| 490208 | e5-d(10)-e5 | 89 | 117 |
| 490209 | e5-d(10)-e5 | 81 | 118 |
| 422086 | e5-d(10)-e5 | 90 | 119 |
| 407935 | e5-d(10)-e5 | 91 | 120 |
| 422087 | e5-d(10)-e5 | 89 | 121 |
| 407936 | e5-d(10)-e5 | 80 | 122 |
| 407939 | e5-d(10)-e5 | 67 | 72 |

e = 2′-MOE, d = 2′-deoxynucleoside

TABLE 19
Inhibition of human Target-X mRNA levels
by chimeric antisense oligonucleotides

| ISIS No | Motif | % inhibition | SEQ CODE |
|---|---|---|---|
| 407662 | e5-d(10)-e5 | 76 | 123 |
| 416446 | e5-d(10)-e5 | 73 | 103 |

e = 2′-MOE, d = 2′-deoxynucleoside

### Example 5: Modified Chimeric Antisense Oligonucleotides Comprising 6′-(S)—CH₃ Bicyclic Nucleoside (e.g cEt) Modifications at 5′ and 3′ Wing Regions Targeting Human Target-X

Additional antisense oligonucleotides were designed targeting a Target-X nucleic acid and were tested for their effects on Target-X mRNA in vitro. ISIS 407939, which was described in an earlier publication (WO 2009/061851) were also tested. ISIS 457851, ISIS 472925, ISIS 472926, ISIS 472935, ISIS 472942, ISIS 472958, ISIS 472959, ISIS 472970, ISIS 472976, ISIS 472983, ISIS 472984, ISIS 472988, ISIS 472991, ISIS 472994, ISIS 472995, ISIS 472996, ISIS 472998, and ISIS 473020, described in the Examples above were also included in the screen.

The newly designed chimeric antisense oligonucleotides in Table 20 were designed as 2-10-2 cEt gapmers. The newly designed gapmers are 14 nucleosides in length, wherein the central gap segment comprises of ten 2′-deoxyribonucleosides and is flanked by wing segments on the 5′ direction and the 3′ direction comprising five nucleosides each. Each nucleoside in the 5′ wing segment and each nucleoside in the 3′ wing segment comprises 6′-(S)—CH₃ bicyclic nucleoside (e.g cEt) modification. The internucleoside linkages throughout each gapmer are phosphorothioate (P═S) linkages. All cytosine residues throughout each gapmer are 5-methylcytosines.

Each gapmer listed in Table 20 is targeted to the human Target-X genomic sequence.

Activity of the newly designed oligonucleotides was compared to ISIS 407939. Cultured Hep3B cells at a density of 20,000 cells per well were transfected using electroporation with 2,000 nM antisense oligonucleotide. After a treatment period of approximately 24 hours, RNA was isolated from the cells and Target-X mRNA levels were measured by quantitative real-time PCR. Human primer probe set RTS2927 (described hereinabove in Example 1) was used to measure mRNA levels. Target-X mRNA levels were adjusted according to total RNA content, as measured by RIBOGREEN. Results are presented as percent inhibition of Target-X, relative to untreated control cells. A total of 614 oligonucleotides were tested. Only those oligonucleotides which were selected for further studies are shown in Table 20. Many of the newly designed antisense oligonucleotides provided in Table 20 achieved greater than 72% inhibition and, therefore, are more potent than ISIS 407939.

TABLE 20
Inhibition of human Target-X mRNA levels by chimeric
antisense oligonucleotides targeted to Target-X

| ISIS No | % inhibition | Motif | Wing Chemistry | SEQ CODE |
|---|---|---|---|---|
| 407939 | 72 | 5-10-5 | cEt | 72 |
| 473020 | 90 | 2-10-2 | cEt | 25 |
| 492465 | 83 | 2-10-2 | cEt | 124 |
| 492467 | 74 | 2-10-2 | cEt | 125 |
| 492492 | 84 | 2-10-2 | cEt | 126 |
| 492494 | 91 | 2-10-2 | cEt | 127 |
| 492503 | 89 | 2-10-2 | cEt | 128 |
| 492530 | 91 | 2-10-2 | cEt | 129 |
| 492534 | 91 | 2-10-2 | cEt | 130 |
| 492536 | 90 | 2-10-2 | cEt | 131 |
| 492541 | 84 | 2-10-2 | cEt | 132 |
| 492545 | 89 | 2-10-2 | cEt | 133 |
| 492566 | 90 | 2-10-2 | cEt | 134 |
| 492571 | 82 | 2-10-2 | cEt | 135 |
| 492572 | 89 | 2-10-2 | cEt | 136 |
| 492573 | 90 | 2-10-2 | cEt | 137 |
| 492574 | 92 | 2-10-2 | cEt | 138 |
| 492575 | 88 | 2-10-2 | cEt | 139 |
| 492593 | 83 | 2-10-2 | cEt | 140 |
| 492617 | 91 | 2-10-2 | cEt | 141 |
| 492618 | 92 | 2-10-2 | cEt | 142 |
| 492619 | 90 | 2-10-2 | cEt | 143 |
| 492621 | 75 | 2-10-2 | cEt | 144 |
| 492104 | 89 | 2-10-2 | cEt | 145 |
| 492105 | 86 | 2-10-2 | cEt | 146 |
| 492189 | 88 | 2-10-2 | cEt | 147 |
| 492194 | 92 | 2-10-2 | cEt | 148 |
| 492195 | 90 | 2-10-2 | cEt | 149 |
| 472925 | 87 | 2-10-2 | cEt | 32 |
| 492196 | 91 | 2-10-2 | cEt | 150 |
| 472926 | 88 | 2-10-2 | cEt | 33 |
| 492205 | 92 | 2-10-2 | cEt | 151 |
| 492215 | 77 | 2-10-2 | cEt | 152 |
| 492221 | 79 | 2-10-2 | cEt | 153 |
| 472935 | 82 | 2-10-2 | cEt | 36 |
| 492234 | 86 | 2-10-2 | cEt | 154 |
| 472942 | 85 | 2-10-2 | cEt | 43 |
| 492276 | 75 | 2-10-2 | cEt | 155 |
| 492277 | 75 | 2-10-2 | cEt | 156 |
| 492306 | 85 | 2-10-2 | cEt | 157 |
| 492317 | 93 | 2-10-2 | cEt | 158 |
| 472958 | 92 | 2-10-2 | cEt | 46 |
| 472959 | 88 | 2-10-2 | cEt | 47 |
| 492329 | 88 | 2-10-2 | cEt | 159 |
| 492331 | 95 | 2-10-2 | cEt | 160 |
| 492333 | 85 | 2-10-2 | cEt | 161 |
| 492334 | 88 | 2-10-2 | cEt | 162 |
| 457851 | 89 | 2-10-2 | cEt | 51 |
| 472970 | 92 | 2-10-2 | cEt | 52 |
| 492365 | 69 | 2-10-2 | cEt | 163 |
| 472976 | 94 | 2-10-2 | cEt | 54 |
| 472983 | 76 | 2-10-2 | cEt | 55 |
| 472984 | 72 | 2-10-2 | cEt | 56 |
| 492377 | 70 | 2-10-2 | cEt | 164 |
| 492380 | 80 | 2-10-2 | cEt | 165 |
| 492384 | 61 | 2-10-2 | cEt | 166 |
| 472988 | 59 | 2-10-2 | cEt | 60 |
| 492388 | 70 | 2-10-2 | cEt | 167 |
| 492389 | 70 | 2-10-2 | cEt | 168 |
| 492390 | 89 | 2-10-2 | cEt | 169 |
| 492391 | 80 | 2-10-2 | cEt | 170 |
| 472991 | 84 | 2-10-2 | cEt | 62 |
| 492398 | 88 | 2-10-2 | cEt | 171 |
| 492399 | 94 | 2-10-2 | cEt | 172 |
| 492401 | 91 | 2-10-2 | cEt | 173 |
| 492403 | 78 | 2-10-2 | cEt | 174 |
| 472994 | 95 | 2-10-2 | cEt | 66 |
| 472995 | 91 | 2-10-2 | cEt | 68 |
| 492404 | 84 | 2-10-2 | cEt | 175 |
| 492405 | 87 | 2-10-2 | cEt | 176 |
| 472996 | 85 | 2-10-2 | cEt | 70 |
| 492406 | 43 | 2-10-2 | cEt | 177 |
| 472998 | 92 | 2-10-2 | cEt | 74 |
| 492440 | 89 | 2-10-2 | cEt | 178 |

### Example 6: Modified Chimeric Antisense Oligonucleotides Comprising 6′-(S)—CH₃ Bicyclic Nucleoside (e.g cEt) Modifications at 5′ and 3′ Wing Regions Targeting Human Target-X

Additional antisense oligonucleotides were designed targeting a Target-X nucleic acid and were tested for their effects on Target-X mRNA in vitro. Also tested was ISIS 407939, a 5-10-5 MOE gapmer targeting human Target-X, which was described in an earlier publication (WO 2009/061851). ISIS 472998 and ISIS 473046, described in the Examples above were also included in the screen.

The newly designed chimeric antisense oligonucleotides in Table 21 were designed as 2-10-2 cEt gapmers. The newly designed gapmers are 14 nucleosides in length, wherein the central gap segment comprises of ten 2′-deoxyribonucleosides and is flanked by wing segments on the 5′ direction and the 3′ direction comprising five nucleosides each. Each nucleoside in the 5′ wing segment and each nucleoside in the 3′ wing segment comprise 6′-(S)—CH₃ bicyclic nucleoside (e.g cEt) modification. The internucleoside linkages throughout each gapmer are phosphorothioate (P═S) linkages. All cytosine residues throughout each gapmer are 5-methylcytosines.

Each gapmer listed in Table 21 is targeted to the human Target-X genomic sequence.

Activity of the newly designed gapmers was compared to ISIS 407939. Cultured Hep3B cells at a density of 20,000 cells per well were transfected using electroporation with 2,000 nM antisense oligonucleotide. After a treatment period of approximately 24 hours, RNA was isolated from the cells and Target-X mRNA levels were measured by quantitative real-time PCR. Human primer probe set RTS2927 (described hereinabove in Example 1) was used to measure mRNA levels. Target-X mRNA levels were adjusted according to total RNA content, as measured by RIBOGREEN. Results are presented as percent inhibition of Target-X, relative to untreated control cells. A total of 757 oligonucleotides were tested. Only those oligonucleotides which were selected for further studies are shown in Table 21. Each of the newly designed antisense oligonucleotides provided in Table 21 achieved greater than 67% inhibition and, therefore, are more potent than 407939.

TABLE 21
Inhibition of human Target-X mRNA levels by chimeric
antisense oligonucleotides targeted to Target-X

| ISIS No | % inhibition | Motif | Wing chemistry | SEQ CODE |
|---|---|---|---|---|
| 407939 | 67 | 5-10-5 | cEt | 72 |
| 492651 | 77 | 2-10-2 | cEt | 179 |
| 492652 | 84 | 2-10-2 | cEt | 180 |
| 492658 | 87 | 2-10-2 | cEt | 181 |
| 492725 | 74 | 2-10-2 | cEt | 182 |
| 492730 | 78 | 2-10-2 | cEt | 183 |
| 492731 | 72 | 2-10-2 | cEt | 184 |
| 492784 | 72 | 2-10-2 | cEt | 185 |
| 492816 | 70 | 2-10-2 | cEt | 186 |
| 492818 | 73 | 2-10-2 | cEt | 187 |
| 492877 | 83 | 2-10-2 | cEt | 188 |
| 492878 | 79 | 2-10-2 | cEt | 189 |
| 492913 | 73 | 2-10-2 | cEt | 190 |
| 492914 | 82 | 2-10-2 | cEt | 191 |
| 492928 | 76 | 5-10-5 | cEt | 192 |
| 492938 | 80 | 2-10-2 | cEt | 193 |
| 492991 | 91 | 2-10-2 | cEt | 194 |
| 492992 | 73 | 2-10-2 | cEt | 195 |
| 493087 | 81 | 2-10-2 | cEt | 196 |
| 493114 | 80 | 2-10-2 | cEt | 197 |
| 493178 | 86 | 2-10-2 | cEt | 198 |
| 493179 | 69 | 2-10-2 | cEt | 199 |
| 493182 | 79 | 2-10-2 | cEt | 200 |
| 493195 | 71 | 2-10-2 | cEt | 201 |
| 473046 | 79 | 2-10-2 | cEt | 35 |
| 493201 | 86 | 2-10-2 | cEt | 202 |
| 493202 | 76 | 2-10-2 | cEt | 203 |
| 493255 | 80 | 2-10-2 | cEt | 204 |
| 493291 | 84 | 2-10-2 | cEt | 205 |
| 493292 | 90 | 2-10-2 | cEt | 206 |
| 493296 | 82 | 2-10-2 | cEt | 207 |
| 493298 | 77 | 2-10-2 | cEt | 208 |
| 493299 | 76 | 5-10-5 | cEt | 209 |
| 493304 | 77 | 2-10-2 | cEt | 210 |
| 493312 | 75 | 2-10-2 | cEt | 211 |
| 493333 | 76 | 2-10-2 | cEt | 212 |
| 472998 | 85 | 2-10-2 | cEt | 74 |

### Example 7: Dose-Dependent Antisense Inhibition of Human Target-X in Hep3B Cells

Antisense oligonucleotides from the studies above, exhibiting in vitro inhibition of Target-X mRNA, were selected and tested at various doses in Hep3B cells. Cells were plated at a density of 20,000 cells per well and transfected using electroporation with 0.67 μM, 2.00 μM, 1.11 μM, and 6.00 μM concentrations of antisense oligonucleotide, as specified in Table 22. After a treatment period of approximately 16 hours, RNA was isolated from the cells and Target-X mRNA levels were measured by quantitative real-time PCR. Human Target-X primer probe set RTS2927 was used to measure mRNA levels. Target-X mRNA levels were adjusted according to total RNA content, as measured by RIBOGREEN®. Results are presented as percent inhibition of Target-X, relative to untreated control cells.

The half maximal inhibitory concentration (IC₅₀) of each oligonucleotide is also presented in Table 22. As illustrated in Table 22, Target-X mRNA levels were reduced in a dose-dependent manner in antisense oligonucleotide treated cells. The data also confirms that several of the newly designed gapmers are more potent than ISIS 407939 of the previous publication.

TABLE 22
Dose-dependent antisense inhibition of human
Target-X in Hep3B cells using electroporation

| | 666.6667 | 2000.0 | 6000.0 | IC₅₀ |
|---|---|---|---|---|
| ISIS No | nM | nM | nM | (μM) |
| 407939 | 47 | 68 | 85 | 0.7 |
| 457851 | 60 | 80 | 93 | <0.6 |
| 472916 | 53 | 80 | 87 | <0.6 |
| 472925 | 62 | 86 | 95 | <0.6 |
| 472926 | 66 | 77 | 85 | <0.6 |
| 472935 | 54 | 84 | 94 | <0.6 |
| 472958 | 66 | 82 | 88 | <0.6 |
| 472959 | 64 | 81 | 93 | <0.6 |
| 472970 | 72 | 87 | 86 | <0.6 |
| 472976 | 78 | 92 | 97 | <0.6 |
| 472994 | 79 | 92 | 96 | <0.6 |
| 472995 | 61 | 82 | 93 | <0.6 |
| 472996 | 73 | 91 | 95 | <0.6 |
| 472998 | 63 | 90 | 95 | <0.6 |
| 473019 | 55 | 80 | 86 | <0.6 |
| 473020 | 61 | 76 | 85 | <0.6 |
| 473046 | 61 | 80 | 94 | <0.6 |
| 473055 | 55 | 84 | 94 | <0.6 |
| 492104 | 53 | 76 | 88 | <0.6 |
| 492105 | 62 | 80 | 90 | <0.6 |
| 492189 | 57 | 80 | 92 | <0.6 |
| 492194 | 57 | 83 | 91 | <0.6 |
| 492195 | 58 | 81 | 95 | <0.6 |
| 492196 | 62 | 86 | 95 | <0.6 |
| 492205 | 62 | 87 | 95 | <0.6 |
| 492215 | 60 | 78 | 89 | <0.6 |
| 492221 | 63 | 76 | 92 | <0.6 |
| 492234 | 51 | 74 | 91 | 0.5 |
| 492276 | 50 | 56 | 95 | 0.8 |
| 492277 | 58 | 73 | 81 | <0.6 |
| 492306 | 61 | 75 | 84 | <0.6 |
| 492317 | 59 | 80 | 93 | <0.6 |
| 492329 | 59 | 70 | 89 | <0.6 |
| 492331 | 69 | 87 | 95 | <0.6 |
| 492333 | 47 | 70 | 85 | 0.7 |
| 492334 | 57 | 77 | 90 | <0.6 |
| 492390 | 72 | 88 | 95 | <0.6 |
| 492399 | 68 | 91 | 96 | <0.6 |
| 492401 | 68 | 89 | 95 | <0.6 |
| 492404 | 65 | 87 | 94 | <0.6 |
| 492405 | 44 | 81 | 90 | 0.7 |
| 492406 | 65 | 82 | 92 | <0.6 |
| 492440 | 50 | 70 | 89 | 0.6 |
| 492465 | 16 | 80 | 79 | 1.4 |
| 492467 | 58 | 77 | 92 | <0.6 |
| 492492 | 45 | 80 | 94 | 0.7 |
| 492494 | 63 | 82 | 93 | <0.6 |
| 492503 | 55 | 81 | 93 | <0.6 |
| 492530 | 70 | 86 | 90 | <0.6 |
| 492534 | 67 | 85 | 91 | <0.6 |
| 492536 | 54 | 81 | 89 | <0.6 |
| 492541 | 54 | 71 | 85 | <0.6 |
| 492545 | 59 | 78 | 89 | <0.6 |
| 492566 | 59 | 84 | 85 | <0.6 |
| 492571 | 52 | 81 | 89 | <0.6 |
| 492572 | 67 | 83 | 90 | <0.6 |
| 492573 | 69 | 83 | 92 | <0.6 |
| 492574 | 65 | 82 | 91 | <0.6 |
| 492575 | 72 | 83 | 91 | <0.6 |
| 492593 | 61 | 78 | 90 | <0.6 |
| 492617 | 62 | 80 | 93 | <0.6 |
| 492618 | 47 | 79 | 94 | 0.6 |
| 492619 | 54 | 82 | 95 | <0.6 |
| 492621 | 44 | 85 | 92 | 0.6 |
| 492651 | 53 | 66 | 91 | 0.6 |
| 492652 | 61 | 78 | 88 | <0.6 |
| 492658 | 59 | 79 | 88 | <0.6 |
| 492725 | 43 | 84 | 89 | 0.6 |
| 492730 | 51 | 87 | 93 | 0.4 |
| 492731 | 46 | 82 | 90 | 0.6 |
| 492784 | 56 | 88 | 96 | <0.6 |
| 492816 | 68 | 89 | 97 | <0.6 |
| 492818 | 64 | 84 | 96 | <0.6 |
| 492877 | 67 | 91 | 93 | <0.6 |
| 492878 | 80 | 89 | 93 | <0.6 |
| 492913 | 53 | 87 | 92 | <0.6 |
| 492914 | 75 | 89 | 96 | <0.6 |
| 492928 | 60 | 83 | 94 | <0.6 |
| 492938 | 70 | 90 | 92 | <0.6 |
| 492991 | 67 | 93 | 99 | <0.6 |
| 492992 | 0 | 82 | 95 | 2.1 |
| 493087 | 54 | 81 | 90 | <0.6 |
| 493114 | 50 | 73 | 90 | 0.6 |
| 493178 | 71 | 88 | 96 | <0.6 |
| 493179 | 47 | 82 | 95 | 0.6 |
| 493182 | 79 | 87 | 91 | <0.6 |
| 493195 | 55 | 78 | 90 | <0.6 |
| 493201 | 87 | 93 | 96 | <0.6 |
| 493202 | 68 | 89 | 94 | <0.6 |
| 493255 | 57 | 79 | 93 | <0.6 |
| 493291 | 57 | 87 | 93 | <0.6 |
| 493292 | 70 | 89 | 93 | <0.6 |
| 493296 | 35 | 84 | 91 | 0.9 |
| 493298 | 57 | 84 | 92 | <0.6 |
| 493299 | 65 | 84 | 93 | <0.6 |
| 493304 | 68 | 86 | 94 | <0.6 |
| 493312 | 53 | 82 | 91 | <0.6 |
| 493333 | 66 | 84 | 87 | <0.6 |

### Example 8: Dose-Dependent Antisense Inhibition of Human Target-X in Hep3B Cells

Additional antisense oligonucleotides from the studies described above, exhibiting in vitro inhibition of Target-X mRNA, were selected and tested at various doses in Hep3B cells. Cells were plated at a density of 20,000 cells per well and transfected using electroporation with 0.67 μM, 2.00 μM, 1.11 μM, and 6.00 μM concentrations of antisense oligonucleotide, as specified in Table 23. After a treatment period of approximately 16 hours, RNA was isolated from the cells and Target-X mRNA levels were measured by quantitative real-time PCR. Human Target-X primer probe set RTS2927 was used to measure mRNA levels. Target-X mRNA levels were adjusted according to total RNA content, as measured by RIBOGREEN®. Results are presented as percent inhibition of Target-X, relative to untreated control cells. As illustrated in Table 23, Target-X mRNA levels were reduced in a dose-dependent manner in antisense oligonucleotide treated cells. The data also confirms that several of the newly designed gapmers are more potent than ISIS 407939.

TABLE 23
Dose-dependent antisense inhibition of human
Target-X in Hep3B cells using electroporation

| | 0.67 | 2.00 | 6.00 | IC₅₀ |
|---|---|---|---|---|
| ISIS No | μM | μM | μM | (μM) |
| 407939 | 52 | 71 | 86 | 0.6 |
| 472983 | 49 | 83 | 97 | 0.5 |
| 472984 | 51 | 82 | 95 | 0.5 |
| 472991 | 49 | 82 | 95 | 0.5 |
| 472998 | 59 | 88 | 96 | <0.6 |
| 492365 | 74 | 91 | 96 | <0.6 |
| 492377 | 56 | 76 | 91 | <0.6 |
| 492380 | 63 | 79 | 95 | <0.6 |
| 492384 | 67 | 84 | 94 | <0.6 |
| 492388 | 69 | 87 | 97 | <0.6 |
| 492389 | 62 | 90 | 96 | <0.6 |
| 492391 | 56 | 84 | 94 | <0.6 |
| 492398 | 63 | 80 | 95 | <0.6 |
| 492403 | 58 | 81 | 91 | <0.6 |

### Example 9: Modified Chimeric Antisense Oligonucleotides Comprising 2′-Methoxyethyl (2′-MOE) Modifications at 5′ and 3′ Wing Regions Targeting Human Target-X

Additional antisense oligonucleotides were designed targeting a Target-X nucleic acid and were tested for their effects on Target-X mRNA in vitro. Also tested were ISIS 403052, ISIS 407939, ISIS 416446, ISIS 416472, ISIS 416507, ISIS 416508, ISIS 422087, ISIS 422096, ISIS 422130, and ISIS 422142 which were described in an earlier publication (WO 2009/061851), incorporated herein by reference. ISIS 490149, ISIS 490197, ISIS 490209, ISIS 490275, ISIS 490277, and ISIS 490424, described in the Examples above, were also included in the screen.

The newly designed chimeric antisense oligonucleotides in Table 24 were designed as 3-10-4 2′-MOE gapmers. These gapmers are 17 nucleosides in length, wherein the central gap segment comprises of ten 2′-deoxyribonucleosides and is flanked by wing segments on the 5′ direction with three nucleosides and the 3′ direction with four nucleosides. Each nucleoside in the 5′ wing segment and each nucleoside in the 3′ wing segment has 2′-MOE modifications. The internucleoside linkages throughout each gapmer are phosphorothioate (P═S) linkages. All cytosine residues throughout each gapmer are 5-methylcytosines.

Each gapmer listed in Table 24 is targeted to the human Target-X genomic sequence.

Activity of the newly designed oligonucleotides was compared to ISIS 403052, ISIS 407939, ISIS 416446, ISIS 416472, ISIS 416507, ISIS 416508, ISIS 422087, ISIS 422096, ISIS 422130, and ISIS 422142. Cultured Hep3B cells at a density of 20,000 cells per well were transfected using electroporation with 2,000 nM antisense oligonucleotide. After a treatment period of approximately 24 hours, RNA was isolated from the cells and Target-X mRNA levels were measured by quantitative real-time PCR. Human primer probe set RTS2927 (described hereinabove in Example 1) was used to measure mRNA levels. Target-X mRNA levels were adjusted according to total RNA content, as measured by RIBOGREEN®. Results are presented as percent inhibition of Target-X, relative to untreated control cells. A total of 272 oligonucleotides were tested. Only those oligonucleotides which were selected for further studies are shown in Table 24. Several of the newly designed antisense oligonucleotides provided in Table 24 are more potent than antisense oligonucleotides from the previous publication.

TABLE 24
Inhibition of human Target-X mRNA levels by chimeric
antisense oligonucleotides targeted to Target-X

| ISIS No | % inhibition | Motif | Wing Chemistry | SEQ CODE |
|---|---|---|---|---|
| 403052 | 51 | 5-10-5 | 2′-MOE | 82 |
| 407939 | 78 | 5-10-5 | 2′-MOE | 72 |
| 416446 | 70 | 5-10-5 | 2′-MOE | 103 |
| 416472 | 79 | 5-10-5 | 2′-MOE | 111 |
| 416507 | 84 | 5-10-5 | 2′-MOE | 97 |
| 416508 | 80 | 5-10-5 | 2′-MOE | 100 |
| 422087 | 89 | 5-10-5 | 2′-MOE | 121 |
| 422096 | 78 | 5-10-5 | 2′-MOE | 219 |
| 422130 | 81 | 5-10-5 | 2′-MOE | 225 |
| 422142 | 84 | 5-10-5 | 2′-MOE | 99 |
| 490275 | 77 | 5-10-5 | 2′-MOE | 90 |
| 513462 | 79 | 3-10-4 | 2′-MOE | 213 |
| 513463 | 81 | 3-10-4 | 2′-MOE | 214 |
| 490277 | 74 | 5-10-5 | 2′-MOE | 91 |
| 513487 | 83 | 3-10-4 | 2′-MOE | 215 |
| 513504 | 81 | 3-10-4 | 2′-MOE | 216 |
| 513507 | 86 | 3-10-4 | 2′-MOE | 217 |
| 513508 | 85 | 3-10-4 | 2′-MOE | 218 |
| 490424 | 69 | 5-10-5 | 2′-MOE | 101 |
| 491122 | 87 | 5-10-5 | 2′-MOE | 220 |
| 513642 | 79 | 3-10-4 | 2′-MOE | 221 |
| 490149 | 71 | 5-10-5 | 2′-MOE | 109 |
| 513419 | 90 | 3-10-4 | 2′-MOE | 222 |
| 513420 | 89 | 3-10-4 | 2′-MOE | 223 |
| 513421 | 88 | 3-10-4 | 2′-MOE | 224 |
| 490197 | 77 | 5-10-5 | 2′-MOE | 116 |
| 513446 | 89 | 3-10-4 | 2′-MOE | 226 |
| 513447 | 83 | 3-10-4 | 2′-MOE | 227 |
| 490209 | 79 | 5-10-5 | 2′-MOE | 118 |
| 513454 | 84 | 3-10-4 | 2′-MOE | 228 |
| 513455 | 92 | 3-10-4 | 2′-MOE | 229 |
| 513456 | 89 | 3-10-4 | 2′-MOE | 230 |
| 513457 | 83 | 3-10-4 | 2′-MOE | 231 |

### Example 10: Dose-Dependent Antisense Inhibition of Human Target-X in Hep3B Cells

Antisense oligonucleotides from the studies above, exhibiting in vitro inhibition of Target-X mRNA, were selected and tested at various doses in Hep3B cells. ISIS 403052, ISIS 407643, ISIS 407935, ISIS 407936, ISIS 407939, ISIS 416446, ISIS 416459, ISIS 416472, ISIS 416507, ISIS 416508, ISIS 416549, ISIS 422086, ISIS 422087, ISIS 422130, ISIS and 422142, 5-10-5 MOE gapmers targeting human Target-X, which were described in an earlier publication (WO 2009/061851).

Cells were plated at a density of 20,000 cells per well and transfected using electroporation with 0.625 μM, 1.25 μM, 2.50 μM, 5.00 μM and 10.00 μM concentrations of antisense oligonucleotide, as specified in Table 25. After a treatment period of approximately 16 hours, RNA was isolated from the cells and Target-X mRNA levels were measured by quantitative real-time PCR. Human Target-X primer probe set RTS2927 was used to measure mRNA levels. Target-X mRNA levels were adjusted according to total RNA content, as measured by RIBOGREEN®. Results are presented as percent inhibition of Target-X, relative to untreated control cells.

The half maximal inhibitory concentration (IC₅₀) of each oligonucleotide is also presented in Table 25. As illustrated in Table 25, Target-X mRNA levels were reduced in a dose-dependent manner in antisense oligonucleotide treated cells. The data also confirms that the newly designed gapmers are potent than gapmers from the previous publication.

TABLE 25
Dose-dependent antisense inhibition of human
Target-X in Hep3B cells using electroporation

| | 0.625 | 1.25 | 2.50 | 5.00 | 10.00 | IC₅₀ |
|---|---|---|---|---|---|---|
| ISIS No | μM | μM | μM | μM | μM | (μM) |
| 403052 | 21 | 35 | 63 | 82 | 89 | 1.9 |
| 407643 | 29 | 46 | 67 | 83 | 90 | 1.4 |
| 407935 | 52 | 68 | 80 | 89 | 91 | <0.6 |
| 407936 | 31 | 51 | 62 | 78 | 84 | 1.4 |
| 407939 | 30 | 61 | 74 | 83 | 88 | 1.0 |
| 416446 | 37 | 53 | 64 | 76 | 83 | 1.2 |
| 416459 | 51 | 76 | 83 | 90 | 92 | <0.6 |
| 416472 | 37 | 52 | 66 | 78 | 85 | 1.2 |
| 416507 | 45 | 68 | 82 | 87 | 90 | 0.7 |
| 416508 | 33 | 56 | 74 | 84 | 89 | 1.1 |
| 416549 | 57 | 71 | 78 | 82 | 85 | <0.6 |
| 422086 | 46 | 67 | 77 | 89 | 92 | 0.7 |
| 422087 | 50 | 69 | 74 | 86 | 91 | 0.6 |
| 422130 | 32 | 65 | 78 | 92 | 93 | 0.9 |
| 422142 | 59 | 73 | 84 | 86 | 88 | <0.6 |
| 490103 | 52 | 57 | 66 | 83 | 88 | 0.9 |
| 490149 | 34 | 58 | 71 | 85 | 91 | 1.0 |
| 490196 | 26 | 59 | 66 | 79 | 84 | 1.3 |
| 490197 | 39 | 63 | 74 | 81 | 90 | 0.8 |
| 490208 | 44 | 70 | 76 | 83 | 88 | 0.6 |
| 490275 | 36 | 58 | 76 | 85 | 89 | 1.0 |
| 490277 | 37 | 63 | 73 | 87 | 87 | 0.8 |
| 490279 | 40 | 54 | 72 | 83 | 89 | 1.0 |
| 490323 | 49 | 68 | 79 | 86 | 90 | <0.6 |
| 490368 | 39 | 62 | 76 | 86 | 91 | 0.8 |
| 490396 | 36 | 53 | 69 | 80 | 87 | 1.1 |
| 490424 | 45 | 65 | 69 | 76 | 82 | 0.6 |
| 490803 | 57 | 74 | 85 | 89 | 92 | <0.6 |
| 513419 | 60 | 71 | 85 | 95 | 96 | <0.6 |
| 513420 | 37 | 69 | 79 | 94 | 96 | 0.7 |
| 513421 | 46 | 64 | 84 | 95 | 97 | 0.6 |
| 513446 | 47 | 81 | 88 | 95 | 96 | <0.6 |
| 513447 | 56 | 74 | 81 | 92 | 96 | <0.6 |
| 513454 | 50 | 77 | 82 | 93 | 95 | <0.6 |
| 513455 | 74 | 82 | 91 | 96 | 96 | <0.6 |
| 513456 | 66 | 80 | 88 | 94 | 95 | <0.6 |
| 513457 | 54 | 67 | 80 | 87 | 89 | <0.6 |
| 513462 | 49 | 72 | 84 | 87 | 89 | <0.6 |
| 513463 | 36 | 62 | 76 | 85 | 89 | 0.9 |
| 513487 | 42 | 56 | 73 | 87 | 93 | 0.9 |
| 513504 | 47 | 65 | 81 | 90 | 91 | 0.6 |
| 513505 | 39 | 50 | 78 | 85 | 92 | 1.0 |
| 513507 | 52 | 73 | 83 | 89 | 93 | <0.6 |
| 513508 | 56 | 78 | 85 | 91 | 94 | <0.6 |

### Example 11: Dose-Dependent Antisense Inhibition of Human Target-X in Hep3B Cells

Additional antisense oligonucleotides from the studies above, exhibiting in vitro inhibition of Target-X mRNA, were tested at various doses in Hep3B cells. ISIS 407935, ISIS 407939, ISIS 416446, ISIS 416472, ISIS 416507, ISIS 416549, ISIS 422086, ISIS 422087, ISIS 422096, and ISIS 422142 5-10-5 MOE gapmers targeting human Target-X, which were described in an earlier publication (WO 2009/061851).

Cells were plated at a density of 20,000 cells per well and transfected using electroporation with 0.3125 μM, 0.625 μM, 1.25 μM, 2.50 μM, 5.00 μM and 10.00 μM concentrations of antisense oligonucleotide, as specified in Table 26. After a treatment period of approximately 16 hours, RNA was isolated from the cells and Target-X mRNA levels were measured by quantitative real-time PCR. Human Target-X primer probe set RTS2927 was used to measure mRNA levels. Target-X mRNA levels were adjusted according to total RNA content, as measured by RIBOGREEN®. Results are presented as percent inhibition of Target-X, relative to untreated control cells. As illustrated in Table 26, Target-X mRNA levels were reduced in a dose-dependent manner in antisense oligonucleotide treated cells. The data also confirms that the newly designed gapmers are more potent than gapmers from the previous publication.

TABLE 26
Dose-dependent antisense inhibition of human
Target-X in Hep3B cells using electroporation

| | 0.3125 | 0.625 | 1.250 | 2.500 | 5.000 | 10.000 | IC₅₀ |
|---|---|---|---|---|---|---|---|
| ISIS No | μM | μM | μM | μM | μM | μM | (μM) |
| 407935 | 30 | 49 | 75 | 86 | 91 | 94 | 0.6 |
| 407939 | 30 | 48 | 61 | 78 | 85 | 90 | 0.8 |
| 416446 | 27 | 52 | 63 | 75 | 85 | 90 | 0.7 |
| 416472 | 38 | 51 | 72 | 83 | 88 | 94 | 0.5 |
| 416507 | 58 | 81 | 76 | 84 | 89 | 92 | <0.3 |
| 416549 | 52 | 67 | 75 | 81 | 88 | 89 | 0.3 |
| 422086 | 48 | 49 | 68 | 78 | 86 | 91 | 0.5 |
| 422087 | 30 | 56 | 66 | 83 | 72 | 92 | 0.6 |
| 422096 | 47 | 63 | 70 | 77 | 83 | 85 | <0.3 |
| 422142 | 69 | 85 | 87 | 85 | 89 | 91 | <0.3 |
| 490103 | 52 | 57 | 68 | 78 | 87 | 93 | 0.4 |
| 490149 | 33 | 64 | 62 | 77 | 86 | 93 | 0.5 |
| 490197 | 38 | 46 | 60 | 75 | 87 | 93 | 0.7 |
| 490208 | 46 | 62 | 73 | 83 | 88 | 91 | 0.4 |
| 490209 | 40 | 54 | 72 | 79 | 85 | 94 | 0.5 |
| 490275 | 52 | 61 | 67 | 78 | 85 | 91 | 0.3 |
| 490277 | 33 | 59 | 77 | 79 | 91 | 94 | 0.5 |
| 490323 | 43 | 61 | 72 | 69 | 84 | 87 | 0.4 |
| 490368 | 50 | 64 | 78 | 83 | 90 | 92 | <0.3 |
| 490396 | 46 | 64 | 68 | 84 | 84 | 90 | 0.3 |
| 490424 | 24 | 47 | 58 | 72 | 76 | 82 | 1.0 |
| 490803 | 45 | 60 | 70 | 84 | 88 | 89 | 0.3 |
| 513419 | 32 | 53 | 76 | 88 | 93 | 95 | 0.5 |
| 513420 | 35 | 59 | 72 | 82 | 94 | 97 | 0.5 |
| 513421 | 46 | 67 | 78 | 86 | 94 | 96 | <0.3 |
| 513446 | 26 | 61 | 77 | 89 | 91 | 97 | 0.5 |
| 513447 | 22 | 48 | 60 | 82 | 91 | 95 | 0.8 |
| 513454 | 25 | 59 | 76 | 86 | 94 | 96 | 0.5 |
| 513455 | 60 | 73 | 85 | 89 | 95 | 96 | <0.3 |
| 513456 | 49 | 60 | 81 | 88 | 94 | 95 | <0.3 |
| 513457 | 43 | 50 | 72 | 77 | 87 | 92 | 0.5 |
| 513462 | 25 | 48 | 58 | 76 | 83 | 88 | 0.8 |
| 513463 | 22 | 45 | 66 | 73 | 85 | 88 | 0.9 |
| 513487 | 41 | 56 | 65 | 79 | 86 | 90 | 0.4 |
| 513504 | 19 | 48 | 63 | 76 | 87 | 92 | 0.9 |
| 513505 | 11 | 21 | 54 | 73 | 85 | 90 | 1.4 |
| 513507 | 47 | 55 | 72 | 82 | 90 | 91 | 0.3 |
| 513508 | 31 | 59 | 74 | 85 | 92 | 93 | 0.5 |
| 513642 | 43 | 55 | 67 | 80 | 88 | 92 | 0.4 |

### Example 12: Tolerability of 2′-MOE Gapmers Targeting Human Target-X in BALB/c Mice

BALB/c mice are a multipurpose mice model, frequently utilized for safety and efficacy testing. The mice were treated with ISIS antisense oligonucleotides selected from studies described above and evaluated for changes in the levels of various plasma chemistry markers.

### Treatment

Groups of male BALB/c mice were injected subcutaneously twice a week for 3 weeks with 50 mg/kg of ISIS 407935, ISIS 416472, ISIS 416549, ISIS 422086, ISIS 422087, ISIS 422096, ISIS 422142, ISIS 490103, ISIS 490149, ISIS 490196, ISIS 490208, ISIS 490209, ISIS 513419, ISIS 513420, ISIS 513421, ISIS 513454, ISIS 513455, ISIS 513456, ISIS 513457, ISIS 513462, ISIS 513463, ISIS 513487, ISIS 513504, ISIS 513508, and ISIS 513642. One group of male BALB/c mice was injected subcutaneously twice a week for 3 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, bilirubin, albumin, and BUN were measured using an automated clinical chemistry analyzer (Hitachi Olympus AU400e, Melville, NY).

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 407935, ISIS 416472, ISIS 416549, ISIS 422087, ISIS 422096, ISIS 490103, ISIS 490196, ISIS 490208, ISIS 513454, ISIS 513455, ISIS 513456, ISIS 513457, ISIS 513487, ISIS 513504, and ISIS 513508 were considered very tolerable in terms of liver function. Based on these criteria, ISIS 422086, ISIS 490209, ISIS 513419, ISIS 513420, and ISIS 513463 were considered tolerable in terms of liver function.

### Example 13: Dose-Dependent Antisense Inhibition of Human Target-X in Hep3B Cells

Additional antisense oligonucleotides from the studies above, exhibiting in vitro inhibition of Target-X mRNA were selected and tested at various doses in Hep3B cells. Also tested was ISIS 407939, a 5-10-5 MOE gapmer, which was described in an earlier publication (WO 2009/061851).

Cells were plated at a density of 20,000 cells per well and transfected using electroporation with 0.074 μM, 0.222 μM, 0.667 μM, 2.000 μM, and 6.000 μM concentrations of antisense oligonucleotide, as specified in Table 27. After a treatment period of approximately 16 hours, RNA was isolated from the cells and Target-X mRNA levels were measured by quantitative real-time PCR. Human Target-X primer probe set RTS2927 (described hereinabove in Example 1) was used to measure mRNA levels. Target-X mRNA levels were adjusted according to total RNA content, as measured by RIBOGREEN®. Results are presented as percent inhibition of Target-X, relative to untreated control cells.

The half maximal inhibitory concentration (IC₅₀) of each oligonucleotide is also presented in Table 27. As illustrated in Table 27, Target-X mRNA levels were reduced in a dose-dependent manner in antisense oligonucleotide treated cells. Many of the newly designed antisense oligonucleotides provided in Table 27 achieved an IC₅₀ of less than 0.9 μM and, therefore, are more potent than ISIS 407939.

TABLE 27
Dose-dependent antisense inhibition of human
Target-X in Hep3B cells using electroporation

| | 0.074 | 0.222 | 0.667 | 2.000 | 6.000 | IC₅₀ |
|---|---|---|---|---|---|---|
| ISIS No | μM | μM | μM | μM | μM | (μM) |
| 407939 | 2 | 17 | 53 | 70 | 87 | 0.9 |
| 472970 | 17 | 47 | 75 | 92 | 95 | 0.3 |
| 472988 | 0 | 8 | 21 | 54 | 92 | 1.4 |
| 472996 | 18 | 59 | 74 | 93 | 95 | 0.2 |
| 473244 | 91 | 95 | 97 | 99 | 99 | <0.07 |
| 473286 | 6 | 53 | 85 | 92 | 98 | 0.3 |
| 473359 | 2 | 3 | 20 | 47 | 67 | 2.6 |
| 473392 | 71 | 85 | 88 | 92 | 96 | <0.07 |
| 473393 | 91 | 96 | 97 | 98 | 99 | <0.07 |
| 473547 | 85 | 88 | 93 | 97 | 98 | <0.07 |
| 473567 | 0 | 25 | 66 | 88 | 95 | 0.7 |
| 473589 | 8 | 47 | 79 | 94 | 99 | 0.3 |
| 482814 | 23 | 68 | 86 | 93 | 96 | 0.1 |
| 482815 | 6 | 48 | 65 | 90 | 96 | 0.4 |
| 482963 | 3 | 68 | 85 | 94 | 96 | 0.2 |
| 483241 | 14 | 33 | 44 | 76 | 93 | 0.6 |
| 483261 | 14 | 21 | 41 | 72 | 88 | 0.7 |
| 483290 | 0 | 1 | 41 | 69 | 92 | 1.0 |
| 483414 | 8 | 1 | 36 | 76 | 91 | 0.9 |
| 483415 | 0 | 40 | 52 | 84 | 94 | 0.6 |
| 484559 | 26 | 51 | 78 | 87 | 97 | 0.2 |
| 484713 | 6 | 5 | 53 | 64 | 88 | 0.9 |

### Example 14: Modified Antisense Oligonucleotides Comprising 2′-O-Methoxyethyl (2′-MOE) and 6′-(S)—CH₃ Bicyclic Nucleoside (e.g cEt) Modifications Targeting Human Target-X

Additional antisense oligonucleotides were designed targeting a Target-X nucleic acid and were tested for their effects on Target-X mRNA in vitro. Also tested was ISIS 407939, a 5-10-5 MOE gapmer targeting human Target-X, which was described in an earlier publication (WO 2009/061851). ISIS 472998, ISIS 492878, and ISIS 493201 and 493182, 2-10-2 cEt gapmers, described in the Examples above were also included in the screen.

The newly designed modified antisense oligonucleotides are 16 nucleotides in length and their motifs are described in Table 28. The chemistry column of Table 28 presents the sugar motif of each oligonucleotide, wherein “e” indicates a 2′-O-methoxyethyl (2′-MOE) nucleoside, “k” indicates a 6′-(S)—CH₃ bicyclic nucleoside (e.g cEt) and “d” indicates a 2′-deoxyribonucleoside. The internucleoside linkages throughout each gapmer are phosphorothioate (P═S) linkages. All cytosine residues throughout each oligonucleotide are 5-methylcytosines.

Each gapmer listed in Table 28 is targeted to the human Target-X genomic sequence.

Activity of newly designed gapmers was compared to ISIS 407939. Cultured Hep3B cells at a density of 20,000 cells per well were transfected using electroporation with 2,000 nM antisense oligonucleotide. After a treatment period of approximately 24 hours, RNA was isolated from the cells and Target-X mRNA levels were measured by quantitative real-time PCR. Human primer probe set RTS2927 was used to measure mRNA levels. Target-X mRNA levels were adjusted according to total RNA content, as measured by RIBOGREEN®. Results are presented as percent inhibition of Target-X, relative to untreated control cells and demonstrate that several of the newly designed gapmers are more potent than ISIS 407939. A total of 685 oligonucleotides were tested. Only those oligonucleotides which were selected for further studies are shown in Table 28.

TABLE 28
Inhibition of human Target-X mRNA levels by chimeric
antisense oligonucleotides targeted to Target-X

| ISIS No | % inhibition | Chemistry | SEQ CODE |
|---|---|---|---|
| 407939 | 68 | eeeee-d(10)-eeeee | 72 |
| 492878 | 73 | kk-d(10)-kk | |
| 493182 | 80 | kk-d(10)-kk | |
| 493201 | 84 | kk-d(10)-kk | |
| 472998 | 91 | kk-d(10)-kk | |
| 515640 | 75 | eee-d(10)-kkk | 23 |
| 515637 | 77 | eee-d(10)-kkk | 232 |
| 515554 | 72 | eee-d(10)-kkk | 233 |
| 515406 | 80 | kkk-d(10)-eee | 234 |
| 515558 | 81 | eee-d(10)-kkk | 234 |
| 515407 | 88 | kkk-d(10)-eee | 235 |
| 515408 | 85 | kkk-d(10)-eee | 236 |
| 515422 | 86 | kkk-d(10)-eee | 237 |
| 515423 | 90 | kkk-d(10)-eee | 238 |
| 515575 | 84 | eee-d(10)-kkk | 238 |
| 515424 | 87 | kkk-d(10)-eee | 239 |
| 515432 | 78 | kkk-d(10)-eee | 240 |
| 515433 | 71 | kkk-d(10)-eee | 241 |
| 515434 | 76 | kkk-d(10)-eee | 242 |
| 515334 | 85 | kkk-d(10)-eee | 243 |
| 515649 | 61 | eee-d(10)-kkk | 88 |
| 515338 | 86 | kkk-d(10)-eee | 244 |
| 515438 | 76 | kkk-d(10)-eee | 245 |
| 515439 | 75 | kkk-d(10)-eee | 246 |
| 516003 | 87 | eee-d(10)-kkk | 247 |
| 515647 | 60 | eee-d(10)-kkk | 77 |
| 515639 | 78 | eee-d(10)-kkk | 34 |
| 493201 | 84 | eee-d(10)-kkk | 202 |
| 515648 | 36 | kkk-d(10)-eee | 248 |
| 515641 | 69 | kk-d(10)-eeee | 39 |
| 515650 | 76 | kkk-d(10)-eee | 44 |
| 515354 | 87 | eee-d(10)-kkk | 249 |
| 515926 | 87 | eee-d(10)-kkk | 250 |
| 515366 | 87 | kk-d(10)-eeee | 251 |
| 515642 | 58 | kkk-d(10)-eee | 252 |
| 515643 | 81 | eee-d(10)-kkk | 53 |
| 515944 | 84 | kk-d(10)-eeee | 253 |
| 515380 | 90 | kkk-d(10)-eee | 254 |
| 515532 | 83 | kkk-d(10)-eee | 254 |
| 515945 | 85 | kk-d(10)-eeee | 254 |
| 515381 | 82 | kk-d(10)-eeee | 255 |
| 515382 | 95 | kkk-d(10)-eee | 256 |
| 515948 | 94 | eee-d(10)-kkk | 256 |
| 515949 | 87 | eee-d(10)-kkk | 257 |
| 515384 | 89 | kkk-d(10)-eee | 258 |
| 515635 | 82 | kk-d(10)-eeee | 65 |
| 515638 | 90 | kkk-d(10)-eee | 67 |
| 515386 | 92 | kk-d(10)-eeee | 259 |
| 515951 | 84 | eee-d(10)-kkk | 259 |
| 515387 | 78 | kkk-d(10)-eee | 260 |
| 515952 | 89 | kkk-d(10)-eee | 260 |
| 515636 | 90 | kkk-d(10)-eee | 69 |
| 515388 | 84 | eee-d(10)-kkk | 261 |

e = 2′-MOE, k = cEt, d = 2′-deoxyribonucleoside

### Example 15: Tolerability of Modified Oligonucleotides Comprising 2′-O-Methoxyethyl (2′-MOE) and 6′-(S)—CH₃ Bicyclic Nucleoside (e.g cEt) Modifications Targeting Human Target-X in BALB/c Mice

BALB/c mice were treated with ISIS antisense oligonucleotides selected from studies described above and evaluated for changes in the levels of various plasma chemistry markers.

Additionally, the newly designed modified antisense oligonucleotides were also added to this screen. The newly designed chimeric antisense oligonucleotides are 16 nucleotides in length and their motifs are described in Table 29. The chemistry column of Table 29 presents the sugar motif of each oligonucleotide, wherein “e” indicates a 2′-O-methoxyethyl (2′-MOE) nucleoside, “k” indicates a 6′-(S)—CH₃ bicyclic nucleoside (e.g cEt) and “d” indicates a 2′-deoxyribonucleoside. The internucleoside linkages throughout each gapmer are phosphorothioate (P═S) linkages. All cytosine residues throughout each oligonucleotide are 5-methylcytosines.

Each gapmer listed in Table 29 is targeted to the human Target-X genomic sequence.

TABLE 29
Modified chimeric antisense oligonucleotides targeted to Target-X

| ISIS No | Chemistry | SEQ CODE |
|---|---|---|
| 516044 | eee-d(10)-kkk | 21 |
| 516045 | eee-d(10)-kkk | 22 |
| 516058 | eee-d(10)-kkk | 26 |
| 516059 | eee-d(10)-kkk | 27 |
| 516060 | eee-d(10)-kkk | 28 |
| 516061 | eee-d(10)-kkk | 29 |
| 516062 | eee-d(10)-kkk | 30 |
| 516046 | eee-d(10)-kkk | 37 |
| 516063 | eee-d(10)-kkk | 38 |
| 516064 | eee-d(10)-kkk | 89 |
| 516065 | eee-d(10)-kkk | 262 |
| 516066 | eee-d(10)-kkk | 263 |
| 516047 | eee-d(10)-kkk | 41 |
| 516048 | eee-d(10)-kkk | 42 |
| 516049 | eee-d(10)-kkk | 81 |
| 516050 | eee-d(10)-kkk | 45 |
| 516051 | eee-d(10)-kkk | 48 |
| 516052 | eee-d(10)-kkk | 49 |
| 515652 | eee-d(10)-kkk | 50 |
| 508039 | eee-d(10)-kkk | 264 |
| 516053 | eee-d(10)-kkk | 265 |
| 515654 | eee-d(10)-kkk | 76 |
| 515656 | eee-d(10)-kkk | 77 |
| 516054 | eee-d(10)-kkk | 57 |
| 516055 | eee-d(10)-kkk | 59 |
| 515655 | eee-d(10)-kkk | 61 |
| 516056 | eee-d(10)-kkk | 63 |
| 516057 | eee-d(10)-kkk | 64 |
| 515653 | eee-d(10)-kkk | 71 |
| 515657 | eee-d(10)-kkk | 73 |

e = 2′-MOE, k = cEt, d = 2′-deoxyribonucleoside

### Treatment

Groups of 4-6-week old male BALB/c mice were injected subcutaneously twice a week for 3 weeks with 50 mg/kg/week of ISIS 457851, ISIS 515635, ISIS 515636, ISIS 515637, ISIS 515638, ISIS 515639, ISIS 515640, ISIS 515641, ISIS 515642, ISIS 515643, ISIS 515647, ISIS 515648, ISIS 515649, ISSI 515650, ISIS 515652, ISIS 515653, ISIS 515654, ISIS 515655, ISIS 515656, ISIS 515657, ISIS 516044, ISIS 516045, ISIS 516046, ISIS 516047, ISIS 516048, ISIS 516049, ISIS 516050, ISIS 516051, ISIS 516052, ISIS 516053, ISIS 516054, ISIS 516055, ISIS 516056, ISIS 516057, ISIS 516058, ISIS 516059, ISIS 516060, ISIS 516061, ISIS 516062, ISIS 516063, ISIS 516064, ISIS 516065, and ISIS 516066. One group of 4-6-week old male BALB/c mice was injected subcutaneously twice a week for 3 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, bilirubin, albumin, and BUN were measured using an automated clinical chemistry analyzer (Hitachi Olympus AU400e, Melville, NY).

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 515636, ISIS 515639, ISIS 515641, ISIS 515642, ISIS 515648, ISIS 515650, ISIS 515652, ISIS 515653, ISIS 515655, ISIS 515657, ISIS 516044, ISIS 516045, ISIS 516047, ISIS 516048, ISIS 516051, ISIS 516052, ISIS 516053, ISIS 516055, ISIS 516056, ISIS 516058, ISIS 516059, ISIS 516060, ISIS 516061, ISIS 516062, ISIS 516063, ISIS 516064, ISIS 516065, and ISIS 516066 were considered very tolerable in terms of liver function. Based on these criteria, ISIS 457851, ISIS 515635, ISIS 515637, ISIS 515638, ISIS 515643, ISIS 515647, ISIS 515649, ISIS 515650, ISIS 515652, ISIS 515654, ISIS 515656, ISIS 516056, and ISIS 516057 were considered tolerable in terms of liver function.

### Example 16: Efficacy of Modified Oligonucleotides Comprising 2′-O-Methoxyethyl (2′-MOE) and 6′-(S)—CH₃ Bicyclic Nucleoside (e.g cEt) Modifications Targeting Human Target-X in Transgenic Mice

Transgenic mice were developed at Taconic farms harboring a Target-X genomic DNA fragment. The mice were treated with ISIS antisense oligonucleotides selected from studies described above and evaluated for efficacy.

### Treatment

Groups of 3-4 male and female transgenic mice were injected subcutaneously twice a week for 3 weeks with 20 mg/kg/week of ISIS 457851, ISIS 515636, ISIS 515639, ISIS 515653, ISIS 516053, ISIS 516065, and ISIS 516066. One group of mice was injected subcutaneously twice a week for 3 weeks with control oligonucleotide, ISIS 141923 (CCTTCCCTGAAGGTTCCTCC, 5-10-5 MOE gapmer with no known murine target, SEQ ID NO: 9). One group of mice was injected subcutaneously twice a week for 3 weeks with PBS. Mice were euthanized 48 hours after the last dose, and organs and plasma were harvested for further analysis.

### RNA Analysis

RNA was extracted from plasma for real-time PCR analysis of Target-X, using primer probe set RTS2927. The mRNA levels were normalized using RIBOGREEN®. Results are presented as percent inhibition of Target-X, relative to control. As shown in Table 30, each of the antisense oligonucleotides achieved reduction of human Target-X mRNA expression over the PBS control. Treatment with the control oligonucleotide did not achieve reduction in Target-X levels, as expected.

TABLE 30
Percent inhibition of Target-X mRNA in transgenic mice

| ISIS No | % inhibition |
|---|---|
| 141923 | 0 |
| 457851 | 76 |
| 515636 | 66 |
| 515639 | 49 |
| 515653 | 78 |
| 516053 | 72 |
| 516065 | 59 |
| 516066 | 39 |

### Protein Analysis

Plasma protein levels of Target-X were estimated using a Target-X ELISA kit (purchased from Hyphen Bio-Med). Results are presented as percent inhibition of Target-X, relative to control. As shown in Table 31, several antisense oligonucleotides achieved reduction of human Target-X protein expression over the PBS control.

TABLE 31
Percent inhibition of Target-X protein levels in transgenic mice

| ISIS No | % inhibition |
|---|---|
| 141923 | 0 |
| 457851 | 64 |
| 515636 | 68 |
| 515639 | 46 |
| 515653 | 0 |
| 516053 | 19 |
| 516065 | 0 |
| 516066 | 7 |

### Example 17: Efficacy of Modified Oligonucleotides Comprising 2′-O-Methoxyethyl (2′-MOE) and 6′-(S)—CH₃ Bicyclic Nucleoside (e.g cEt) Modifications Targeting Human Target-X in Transgenic Mice

Transgenic mice were treated with ISIS antisense oligonucleotides selected from studies described above and evaluated for efficacy.

### Treatment

Groups of 2-4 male and female transgenic mice were injected subcutaneously twice a week for 3 weeks with 10 mg/kg/week of ISIS 407935, ISIS 416472, ISIS 416549, ISIS 422087, ISIS 422096, ISIS 473137, ISIS 473244, ISIS 473326, ISIS 473327, ISIS 473359, ISIS 473392, ISIS 473393, ISIS 473547, ISIS 473567, ISIS 473589, ISIS 473630, ISIS 484559, ISIS 484713, ISIS 490103, ISIS 490196, ISIS 490208, ISIS 513419, ISIS 513454, ISIS 513455, ISIS 513456, ISIS 513457, ISIS 513487, ISIS 513508, ISIS 515640, ISIS 515641, ISIS 515642, ISIS 515648, ISIS 515655, ISIS 515657, ISIS 516045, ISIS 516046, ISIS 516047, ISIS 516048, ISIS 516051, ISIS 516052, ISIS 516055, ISIS 516056, ISIS 516059, ISIS 516061, ISIS 516062, and ISIS 516063. One group of mice was injected subcutaneously twice a week for 3 weeks with PBS. Mice were euthanized 48 hours after the last dose, and organs and plasma were harvested for further analysis.

### Protein Analysis

Plasma protein levels of Target-X were estimated using a Target-X ELISA kit (purchased from Hyphen Bio-Med). Results are presented as percent inhibition of Target-X, relative to control. As shown in Table 32, several antisense oligonucleotides achieved reduction of human Target-X over the PBS control.

TABLE 32
Percent inhibition of Target-X plasma
protein levels in transgenic mice

| ISIS No | % inhibition |
|---|---|
| 407935 | 80 |
| 416472 | 49 |
| 416549 | 29 |
| 422087 | 12 |
| 422096 | 21 |
| 473137 | 57 |
| 473244 | 67 |
| 473326 | 42 |
| 473327 | 100 |
| 473359 | 0 |
| 473392 | 22 |
| 473393 | 32 |
| 473547 | 73 |
| 473567 | 77 |
| 473589 | 96 |
| 473630 | 75 |
| 484559 | 75 |
| 484713 | 56 |
| 490103 | 0 |
| 490196 | 74 |
| 490208 | 90 |
| 513419 | 90 |
| 513454 | 83 |
| 513455 | 91 |
| 513456 | 81 |
| 513457 | 12 |
| 513487 | 74 |
| 513508 | 77 |
| 515640 | 83 |
| 515641 | 87 |
| 515642 | 23 |
| 515648 | 32 |
| 515655 | 79 |
| 515657 | 81 |
| 516045 | 52 |
| 516046 | 79 |
| 516047 | 65 |
| 516048 | 79 |
| 516051 | 84 |
| 516052 | 72 |
| 516055 | 70 |
| 516056 | 0 |
| 516059 | 39 |
| 516061 | 64 |
| 516062 | 96 |
| 516063 | 24 |

### Example 18: Dose-Dependent Antisense Inhibition of Human Target-X in Hep3B Cells

Antisense oligonucleotides exhibiting in vitro inhibition of Target-X mRNA were selected and tested at various doses in Hep3B cells. Also tested was ISIS 407939, a 5-10-5 MOE gapmer targeting human Target-X, which was described in an earlier publication (WO 2009/061851).

Cells were plated at a density of 20,000 cells per well and transfected using electroporation with 0.074 μM, 0.222 μM, 0.667 μM, 2.000 μM, and 6.000 μM concentrations of antisense oligonucleotide, as specified in Table 33. After a treatment period of approximately 16 hours, RNA was isolated from the cells and Target-X mRNA levels were measured by quantitative real-time PCR. Human Target-X primer probe set RTS2927 (described hereinabove in Example 1) was used to measure mRNA levels. Target-X mRNA levels were adjusted according to total RNA content, as measured by RIBOGREEN®. Results are presented as percent inhibition of Target-X, relative to untreated control cells.

The half maximal inhibitory concentration (IC₅₀) of each oligonucleotide is also presented in Table 33. As illustrated in Table 33, Target-X mRNA levels were reduced in a dose-dependent manner in antisense oligonucleotide treated cells. Many of the newly designed antisense oligonucleotides provided in Table 33 achieved an IC₅₀ of less than 2.0 μM and, therefore, are more potent than ISIS 407939.

TABLE 33
Dose-dependent antisense inhibition of human
Target-X in Hep3B cells using electroporation

| | 0.074 | 0.222 | 0.667 | 2.000 | 6.000 | IC₅₀ |
|---|---|---|---|---|---|---|
| ISIS No | μM | μM | μM | μM | μM | (μM) |
| 407939 | 0 | 9 | 21 | 58 | 76 | 2.0 |
| 515636 | 14 | 32 | 50 | 62 | 81 | 0.7 |
| 515639 | 10 | 24 | 41 | 61 | 67 | 1.3 |
| 515640 | 4 | 16 | 35 | 52 | 63 | 2.0 |
| 515641 | 0 | 21 | 27 | 55 | 66 | 1.9 |
| 515642 | 3 | 13 | 36 | 44 | 66 | 2.2 |
| 515648 | 8 | 10 | 10 | 5 | 16 | >6.0 |
| 515653 | 9 | 35 | 26 | 55 | 71 | 1.5 |
| 515655 | 0 | 0 | 6 | 13 | 42 | >6.0 |
| 515657 | 0 | 13 | 17 | 38 | 51 | 6.0 |
| 516045 | 0 | 6 | 15 | 19 | 40 | >6.0 |
| 516046 | 0 | 7 | 32 | 48 | 69 | 2.1 |
| 516047 | 12 | 27 | 41 | 50 | 63 | 1.8 |
| 516051 | 9 | 8 | 34 | 52 | 66 | 2.0 |
| 516052 | 17 | 42 | 27 | 53 | 75 | 1.2 |
| 516053 | 9 | 7 | 28 | 63 | 77 | 1.3 |
| 516055 | 0 | 3 | 27 | 54 | 75 | 2.0 |
| 516056 | 0 | 4 | 14 | 52 | 66 | 2.6 |
| 516057 | 0 | 34 | 33 | 51 | 70 | 1.6 |
| 516058 | 13 | 12 | 25 | 47 | 74 | 2.0 |
| 516059 | 4 | 15 | 36 | 47 | 68 | 1.9 |
| 516060 | 0 | 1 | 39 | 29 | 63 | 3.2 |
| 516061 | 0 | 0 | 24 | 0 | 3 | <6.0 |
| 516062 | 0 | 20 | 43 | 65 | 78 | 1.0 |
| 516063 | 0 | 8 | 10 | 37 | 61 | 3.8 |
| 516064 | 0 | 3 | 13 | 45 | 69 | 2.7 |
| 516065 | 0 | 14 | 38 | 63 | 76 | 1.3 |
| 516066 | 0 | 3 | 30 | 55 | 75 | 1.7 |

### Example 19: Modified Oligonucleotides Comprising 2′-O-Methoxyethyl (2′-MOE) and 6′-(S)—CH₃ Bicyclic Nucleoside (e.g cEt) Modifications Targeting Human Target-X

Additional antisense oligonucleotides were designed targeting a Target-X nucleic acid and were tested for their effects on Target-X mRNA in vitro. ISIS 472998, ISIS 515652, ISIS 515653, ISIS 515654, ISIS 515655, ISIS 515656, and ISIS 515657, described in the Examples above were also included in the screen.

The newly designed chimeric antisense oligonucleotides are 16 or 17 nucleotides in length and their motifs are described in Table 34. The chemistry column of Table 34 presents the sugar motif of each oligonucleotide, wherein “e” indicates a 2′-O-methoxyethyl (2′-MOE) nucleoside, “k” indicates a 6′-(S)—CH₃ bicyclic nucleoside (e.g cEt) and “d” indicates a 2′-deoxyribonucleoside. The internucleoside linkages throughout each gapmer are phosphorothioate (P═S) linkages. All cytosine residues throughout each oligonucleotide are 5-methylcytosines.

Each gapmer listed in Table 34 is targeted to the human Target-X genomic sequence.

Activity of newly designed gapmers was compared to ISIS 407939. Cultured Hep3B cells at a density of 20,000 cells per well were transfected using electroporation with 2,000 nM antisense oligonucleotide. After a treatment period of approximately 24 hours, RNA was isolated from the cells and Target-X mRNA levels were measured by quantitative real-time PCR. Human primer probe set RTS2927 (described hereinabove in Example 1) was used to measure mRNA levels. Target-X mRNA levels were adjusted according to total RNA content, as measured by RIBOGREEN®. Results are presented as percent inhibition of Target-X, relative to untreated control cells.

TABLE 34
Inhibition of human Target-X mRNA levels by chimeric
antisense oligonucleotides targeted to Target-X

| ISIS No | % inhibition | Chemistry | SEQ CODE |
|---|---|---|---|
| 472998 | 85 | kk-d(10)-kk | 74 |
| 515652 | 63 | eee-d(10)-kkk | 50 |
| 515653 | 67 | eee-d(10)-kkk | 71 |
| 515654 | 78 | eee-d(10)-kkk | 86 |
| 515655 | 41 | eee-d(10)-kkk | 61 |
| 515656 | 74 | eee-d(10)-kkk | 87 |
| 515657 | 49 | eee-d(10)-kkk | 73 |
| 529265 | 52 | eek-d(10)-keke | 267 |
| 529332 | 82 | eek-d(10)-keke | 268 |
| 529334 | 78 | eek-d(10)-keke | 269 |
| 529186 | 85 | eek-d(10)-keke | 213 |
| 529223 | 81 | eek-d(10)-kkke | 213 |
| 529129 | 75 | eee-d(10)-kkk | 270 |
| 529149 | 82 | kkk-d(10)-eee | 270 |
| 529177 | 77 | eek-d(10)-keke | 214 |
| 529214 | 78 | eek-d(10)-kkke | 214 |
| 529178 | 79 | eek-d(10)-keke | 271 |
| 529215 | 82 | eek-d(10)-kkke | 271 |
| 529179 | 71 | eek-d(10)-keke | 272 |
| 529216 | 77 | eek-d(10)-kkke | 272 |
| 529193 | 69 | eek-d(10)-keke | 273 |
| 529230 | 70 | eek-d(10)-kkke | 273 |
| 529136 | 48 | eee-d(10)-kkk | 274 |
| 529156 | 68 | kkk-d(10)-eee | 274 |
| 529194 | 44 | eek-d(10)-keke | 275 |
| 529231 | 56 | eek-d(10)-kkke | 275 |
| 529137 | 34 | eee-d(10)-kkk | 276 |
| 529157 | 79 | kkk-d(10)-eee | 276 |
| 529336 | 57 | eek-d(10)-keke | 277 |
| 529338 | 73 | eek-d(10)-keke | 278 |
| 529195 | 55 | eek-d(10)-keke | 279 |
| 529232 | 68 | eek-d(10)-kkke | 279 |
| 529340 | 65 | eek-d(10)-keke | 280 |
| 529342 | 69 | eek-d(10)-keke | 281 |
| 529812 | 69 | k-d(10)-kekee | 282 |
| 529831 | 62 | k-d(10)-kdkee | 282 |
| 529733 | 64 | ke-d(10)-keke | 283 |
| 529753 | 52 | ek-d(10)-keke | 283 |
| 529773 | 57 | ke-d(10)-kdke | 283 |
| 529793 | 36 | ek-d(10)-kdke | 283 |
| 529862 | 48 | kde-d(10)-kdke | 284 |
| 529882 | 35 | edk-d(10)-kdke | 284 |
| 529902 | 44 | k-(d4)-k-(d4)-k-(d4)-ke | 284 |
| 529559 | 71 | eek-d(10)-kke | 26 |
| 529584 | 57 | kee-d(10)-kke | 26 |
| 529609 | 58 | edk-d(10)-kke | 26 |
| 529634 | 49 | kde-d(10)-kke | 26 |
| 529659 | 52 | kddk-d(9)-kke | 26 |
| 529684 | 48 | kdde-d(9)-kke | 26 |
| 529709 | 61 | eddk-d(9)-kke | 26 |
| 529922 | 52 | eeee-d(9)-kke | 26 |
| 529344 | 50 | eek-d(10)-keke | 285 |
| 529138 | 32 | eee-d(10)-kkk | 286 |
| 529158 | 75 | kkk-d(10)-eee | 286 |
| 529184 | 75 | eek-d(10)-keke | 215 |
| 529221 | 78 | eek-d(10)-kkke | 215 |
| 529127 | 67 | eee-d(10)-kkk | 287 |
| 529147 | 79 | kkk-d(10)-eee | 287 |
| 529346 | 58 | eek-d(10)-keke | 288 |
| 529348 | 65 | eek-d(10)-keke | 289 |
| 529350 | 77 | eek-d(10)-keke | 290 |
| 529813 | 20 | k-d(10)-kekee | 291 |
| 529832 | 47 | k-d(10)-kdkee | 291 |
| 529734 | 63 | ke-d(10)-keke | 292 |
| 529754 | 58 | ek-d(10)-keke | 292 |
| 529774 | 49 | ke-d(10)-kdke | 292 |
| 529794 | 51 | ek-d(10)-kdke | 292 |
| 529863 | 64 | kde-d(10)-kdke | 293 |
| 529883 | 78 | edk-d(10)-kdke | 293 |
| 529903 | 36 | k-d(4)-k-d(4)-k-d(4)-ke | 293 |
| 529560 | 71 | eek-d(10)-kke | 27 |
| 529585 | 70 | kee-d(10)-kke | 27 |
| 529610 | 66 | edk-d(10)-kke | 27 |
| 529635 | 45 | kde-d(10)-kke | 27 |
| 529660 | 53 | kddk-d(9)-kke | 27 |
| 529685 | 42 | kdde-d(9)-kke | 27 |
| 529710 | 60 | eddk-d(9)-kke | 27 |
| 529923 | 63 | eeee-d(9)-kke | 27 |
| 529196 | 74 | eek-d(10)-keke | 294 |
| 529233 | 80 | eek-d(10)-kkke | 294 |
| 529139 | 75 | eee-d(10)-kkk | 295 |
| 529159 | 62 | kkk-d(10)-eee | 295 |
| 529352 | 74 | eek-d(10)-keke | 296 |
| 529354 | 67 | eek-d(10)-keke | 297 |
| 529197 | 43 | eek-d(10)-keke | 298 |
| 529234 | 58 | eek-d(10)-kkke | 298 |
| 529140 | 29 | eee-d(10)-kkk | 299 |
| 529160 | 59 | kkk-d(10)-eee | 299 |
| 529180 | 80 | eek-d(10)-keke | 216 |
| 529217 | 79 | eek-d(10)-kkke | 216 |
| 529814 | 51 | k-d(10)-kekee | 300 |
| 529833 | 52 | k-d(10)-kdkee | 300 |
| 529735 | 43 | ke-d(10)-keke | 301 |
| 529755 | 60 | ek-d(10)-keke | 301 |
| 529775 | 38 | ke-d(10)-kdke | 301 |
| 529795 | 58 | ek-d(10)-kdke | 301 |
| 529864 | 41 | kde-d(10)-kdke | 302 |
| 529884 | 48 | edk-d(10)-kdke | 302 |
| 529904 | 44 | k-d(4)-k-(d4)-k-d(4)-ke | 302 |
| 529934 | 61 | eek-d(10)-keke | 302 |
| 529356 | 71 | eek-d(10)-keke | 303 |
| 529561 | 75 | eek-d(10)-kke | 28 |
| 529586 | 65 | kee-d(10)-kke | 28 |
| 529611 | 54 | edk-d(10)-kke | 28 |
| 529636 | 39 | kde-d(10)-kke | 28 |
| 529661 | 67 | kddk-d(9)-kke | 28 |
| 529686 | 66 | kdde-d(9)-kke | 28 |
| 529711 | 60 | eddk-d(9)-kke | 28 |
| 529924 | 62 | eeee-d(9)-kke | 28 |
| 529358 | 82 | eek-d(10)-keke | 304 |
| 529181 | 79 | eek-d(10)-keke | 217 |
| 529218 | 73 | eek-d(10)-kkke | 217 |
| 529182 | 85 | eek-d(10)-keke | 218 |
| 529219 | 84 | eek-d(10)-kkke | 218 |
| 529360 | 84 | eek-d(10)-keke | 305 |
| 529362 | 87 | eek-d(10)-keke | 306 |
| 529364 | 81 | eek-d(10)-keke | 307 |
| 529366 | 77 | eek-d(10)-keke | 308 |
| 529198 | 28 | eek-d(10)-keke | 309 |
| 529235 | 8 | eek-d(10)-kkke | 309 |
| 529141 | 34 | eee-d(10)-kkk | 310 |
| 529161 | 66 | kkk-d(10)-eee | 310 |
| 529368 | 27 | eek-d(10)-keke | 311 |
| 529370 | 44 | eek-d(10)-keke | 312 |
| 529372 | 61 | eek-d(10)-keke | 313 |
| 529374 | 71 | eek-d(10)-keke | 314 |
| 529376 | 63 | eek-d(10)-keke | 315 |
| 529378 | 68 | eek-d(10)-keke | 316 |
| 529380 | 79 | eek-d(10)-keke | 317 |
| 529382 | 77 | eek-d(10)-keke | 318 |
| 529384 | 75 | eek-d(10)-keke | 319 |
| 529386 | 40 | eek-d(10)-keke | 320 |
| 529240 | 73 | eek-d(10)-keke | 321 |
| 529241 | 67 | eek-d(10)-keke | 322 |
| 529242 | 42 | eek-d(10)-keke | 323 |
| 529243 | 60 | eek-d(10)-keke | 324 |
| 529388 | 65 | eek-d(10)-keke | 325 |
| 529815 | 37 | k-d(10)-kekee | 326 |
| 529834 | 44 | k-d(10)-kdkee | 326 |
| 529736 | 47 | ke-d(10)-keke | 327 |
| 529756 | 78 | ek-d(10)-keke | 327 |
| 529776 | 37 | ke-d(10)-kdke | 327 |
| 529796 | 71 | ek-d(10)-kdke | 327 |
| 529865 | 70 | kde-d(10)-kdke | 328 |
| 529885 | 59 | edk-d(10)-kdke | 328 |
| 529905 | 54 | k-(d4)-k-(d4)-k-(d4)-ke | 328 |
| 529935 | 70 | eek-d(10)-keke | 328 |
| 529562 | 87 | eek-d(10)-kke | 29 |
| 529587 | 68 | kee-d(10)-kke | 29 |
| 529612 | 67 | edk-d(10)-kke | 29 |
| 529637 | 64 | kde-d(10)-kke | 29 |
| 529662 | 62 | kddk-d(9)-kke | 29 |
| 529687 | 63 | kdde-d(9)-kke | 29 |
| 529712 | 61 | eddk-d(9)-kke | 29 |
| 529925 | 61 | eeee-d(9)-kke | 29 |
| 529816 | 77 | k-d(10)-kekee | 329 |
| 529835 | 80 | k-d(10)-kdkee | 329 |
| 529737 | 82 | ke-d(10)-keke | 330 |
| 529757 | 83 | ek-d(10)-keke | 330 |
| 529777 | 68 | ke-d(10)-kdke | 330 |
| 529797 | 77 | ek-d(10)-kdke | 330 |
| 529866 | 15 | kde-d(10)-kdke | 331 |
| 529886 | 71 | edk-d(10)-kdke | 331 |
| 529906 | 63 | k-(d4)-k-(d4)-k-(d4)-ke | 331 |
| 529936 | 78 | eek-d(10)-keke | 331 |
| 529563 | 89 | eek-d(10)-kke | 30 |
| 529588 | 84 | kee-d(10)-kke | 30 |
| 529613 | 80 | edk-d(10)-kke | 30 |
| 529638 | 48 | kde-d(10)-kke | 30 |
| 529663 | 85 | kddk-d(9)-kke | 30 |
| 529688 | 42 | kdde-d(9)-kke | 30 |
| 529713 | 81 | eddk-d(9)-kke | 30 |
| 529926 | 67 | eeee-d(9)-kke | 30 |
| 529390 | 53 | eek-d(10)-keke | 332 |
| 529392 | 63 | eek-d(10)-keke | 333 |
| 529394 | 58 | eek-d(10)-keke | 334 |
| 529396 | 56 | eek-d(10)-keke | 335 |
| 529398 | 62 | eek-d(10)-keke | 336 |
| 529400 | 44 | eek-d(10)-keke | 337 |
| 529402 | 39 | eek-d(10)-keke | 338 |
| 529404 | 46 | eek-d(10)-keke | 339 |
| 529406 | 63 | eek-d(10)-keke | 340 |
| 529244 | 58 | eek-d(10)-keke | 341 |
| 529245 | 68 | eek-d(10)-keke | 342 |
| 529246 | 60 | eek-d(10)-keke | 343 |
| 529247 | 36 | eek-d(10)-keke | 344 |
| 529248 | 43 | eek-d(10)-keke | 345 |
| 529249 | 23 | eek-d(10)-keke | 346 |
| 529250 | 69 | eek-d(10)-keke | 347 |
| 529251 | 15 | eek-d(10)-keke | 348 |
| 529252 | 44 | eek-d(10)-keke | 349 |
| 529253 | 42 | eek-d(10)-keke | 350 |
| 529408 | 67 | eek-d(10)-keke | 351 |
| 529410 | 19 | eek-d(10)-keke | 352 |
| 529412 | 57 | eek-d(10)-keke | 353 |
| 529414 | 80 | eek-d(10)-keke | 354 |
| 529416 | 85 | eek-d(10)-keke | 355 |
| 529418 | 70 | eek-d(10)-keke | 356 |
| 529420 | 78 | eek-d(10)-keke | 357 |
| 529422 | 19 | eek-d(10)-keke | 358 |
| 529424 | 48 | eek-d(10)-keke | 359 |
| 529426 | 66 | eek-d(10)-keke | 360 |
| 529428 | 59 | eek-d(10)-keke | 361 |
| 529430 | 83 | eek-d(10)-keke | 362 |
| 529432 | 84 | eek-d(10)-keke | 363 |
| 529199 | 71 | eek-d(10)-keke | 364 |
| 529236 | 76 | eek-d(10)-kkke | 364 |
| 529142 | 64 | eee-d(10)-kkk | 365 |
| 529162 | 60 | kkk-d(10)-eee | 365 |
| 529254 | 46 | eek-d(10)-keke | 366 |
| 529255 | 52 | eek-d(10)-keke | 367 |
| 529256 | 57 | eek-d(10)-keke | 368 |
| 529257 | 55 | eek-d(10)-keke | 369 |
| 529258 | 3 | eek-d(10)-keke | 370 |
| 529259 | 71 | eek-d(10)-keke | 371 |
| 529260 | 72 | eek-d(10)-keke | 372 |
| 529261 | 56 | eek-d(10)-keke | 373 |
| 529262 | 56 | eek-d(10)-keke | 374 |
| 529263 | 59 | eek-d(10)-keke | 375 |
| 529264 | 49 | eek-d(10)-keke | 376 |
| 529434 | 83 | eek-d(10)-keke | 377 |
| 529436 | 80 | eek-d(10)-keke | 378 |
| 529438 | 79 | eek-d(10)-keke | 379 |
| 529440 | 87 | eek-d(10)-keke | 380 |
| 529442 | 68 | eek-d(10)-keke | 381 |
| 529443 | 72 | eek-d(10)-keke | 382 |
| 529444 | 68 | eek-d(10)-keke | 383 |
| 529445 | 85 | eek-d(10)-keke | 384 |
| 529446 | 72 | eek-d(10)-keke | 385 |
| 529447 | 60 | eek-d(10)-keke | 386 |
| 529448 | 77 | eek-d(10)-keke | 387 |
| 529807 | 78 | k-d(10)-kekee | 388 |
| 529826 | 61 | k-d(10)-kdkee | 388 |
| 529449 | 81 | eek-d(10)-keke | 389 |
| 529728 | 75 | ke-d(10)-keke | 390 |
| 529748 | 80 | ek-d(10)-keke | 390 |
| 529768 | 68 | ke-d(10)-kdke | 390 |
| 529788 | 74 | ek-d(10)-kdke | 390 |
| 529857 | 67 | kde-d(10)-kdke | 389 |
| 529877 | 77 | edk-d(10)-kdke | 389 |
| 529897 | 26 | k-(d4)-k-(d4)-k-(d4)-ke | 389 |
| 529200 | 78 | eek-d(10)-keke | 391 |
| 529237 | 84 | eek-d(10)-kkke | 391 |
| 529564 | 90 | eek-d(10)-kke | 34 |
| 529589 | 86 | kee-d(10)-kke | 34 |
| 529614 | 82 | edk-d(10)-kke | 34 |
| 529639 | 80 | kde-d(10)-kke | 34 |
| 529664 | 69 | kddk-d(9)-kke | 34 |
| 529689 | 71 | kdde-d(9)-kke | 34 |
| 529714 | 73 | eddk-d(9)-kke | 34 |
| 529917 | 73 | eeee-d(9)-kke | 34 |
| 529143 | 68 | eee-d(10)-kkk | 392 |
| 529163 | 50 | kkk-d(10)-eee | 392 |
| 529201 | 76 | eek-d(10)-keke | 393 |
| 529238 | 72 | eek-d(10)-kkke | 393 |
| 529144 | 57 | eee-d(10)-kkk | 394 |
| 529164 | 71 | kkk-d(10)-eee | 394 |
| 529450 | 91 | eek-d(10)-keke | 395 |
| 529451 | 85 | eek-d(10)-keke | 396 |
| 529266 | 63 | eek-d(10)-keke | 397 |
| 529806 | 52 | k-d(10)-kekee | 398 |
| 529825 | 44 | k-d(10)-kdkee | 398 |
| 529267 | 56 | eek-d(10)-keke | 399 |
| 529727 | 67 | ke-d(10)-keke | 400 |
| 529747 | 63 | ek-d(10)-keke | 400 |
| 529767 | 67 | ke-d(10)-kdke | 400 |
| 529787 | 68 | ek-d(10)-kdke | 400 |
| 529856 | 42 | kde-d(10)-kdke | 399 |
| 529876 | 36 | edk-d(10)-kdke | 399 |
| 529896 | 56 | k-(d4)-k-(d4)-k-(d4)-ke | 399 |
| 529546 | 65 | eek-d(10)-kke | 248 |
| 529571 | 80 | kee-d(10)-kke | 248 |
| 529596 | 43 | edk-d(10)-kke | 248 |
| 529621 | 38 | kde-d(10)-kke | 248 |
| 529646 | 68 | kddk-d(9)-kke | 248 |
| 529671 | 50 | kdde-d(9)-kke | 248 |
| 529696 | 53 | eddk-d(9)-kke | 248 |
| 529916 | 22 | eeee-d(9)-kke | 248 |
| 529547 | 86 | eek-d(10)-kke | 37 |
| 529572 | 75 | kee-d(10)-kke | 37 |
| 529597 | 58 | edk-d(10)-kke | 37 |
| 529622 | 58 | kde-d(10)-kke | 37 |
| 529647 | 18 | kddk-d(9)-kke | 37 |
| 529672 | 23 | kdde-d(9)-kke | 37 |
| 529697 | 28 | eddk-d(9)-kke | 37 |
| 529928 | 36 | eeee-d(9)-kke | 37 |
| 529452 | 63 | eek-d(10)-keke | 401 |
| 529453 | 73 | eek-d(10)-keke | 402 |
| 529454 | 82 | eek-d(10)-keke | 403 |
| 529455 | 84 | eek-d(10)-keke | 404 |
| 529202 | 61 | eek-d(10)-keke | 405 |
| 529239 | 59 | eek-d(10)-kkke | 405 |
| 529145 | 54 | eee-d(10)-kkk | 406 |
| 529165 | 77 | kkk-d(10)-eee | 406 |
| 529456 | 69 | eek-d(10)-keke | 407 |
| 529457 | 81 | eek-d(10)-keke | 408 |
| 529458 | 72 | eek-d(10)-keke | 409 |
| 529459 | 86 | eek-d(10)-keke | 410 |
| 529460 | 88 | eek-d(10)-keke | 411 |
| 529817 | 46 | k-d(10)-kekee | 412 |
| 529836 | 49 | k-d(10)-kdkee | 412 |
| 529738 | 51 | ke-d(10)-keke | 413 |
| 529758 | 53 | ek-d(10)-keke | 413 |
| 529778 | 39 | ke-d(10)-kdke | 413 |
| 529798 | 52 | ek-d(10)-kdke | 413 |
| 529867 | 56 | kde-d(10)-kdke | 414 |
| 529887 | 68 | edk-d(10)-kdke | 414 |
| 529907 | 28 | k-(d4)-k-(d4)-k-(d4)-ke | 414 |
| 529938 | 64 | eek-d(10)-keke | 414 |
| 529565 | 81 | eek-d(10)-kke | 38 |
| 529590 | 49 | kee-d(10)-kke | 38 |
| 529615 | 65 | edk-d(10)-kke | 38 |
| 529640 | 54 | kde-d(10)-kke | 38 |
| 529665 | 77 | kddk-d(9)-kke | 38 |
| 529690 | 77 | kdde-d(9)-kke | 38 |
| 529715 | 63 | eddk-d(9)-kke | 38 |
| 529927 | 62 | eeee-d(9)-kke | 38 |
| 529185 | 66 | eek-d(10)-keke | 221 |
| 529222 | 62 | eek-d(10)-kkke | 221 |
| 529808 | 75 | k-d(10)-kekee | 89 |
| 529827 | 67 | k-d(10)-kdkee | 89 |
| 529128 | 64 | eee-d(10)-kkk | 415 |
| 529148 | 78 | kkk-d(10)-eee | 415 |
| 529461 | 87 | eek-d(10)-keke | 416 |
| 529729 | 71 | ke-d(10)-keke | 415 |
| 529749 | 83 | ek-d(10)-keke | 415 |
| 529769 | 63 | ke-d(10)-kdke | 415 |
| 529789 | 10 | ek-d(10)-kdke | 415 |
| 529800 | 69 | k-d(10)-kekee | 415 |
| 529819 | 78 | k-d(10)-kdkee | 415 |
| 529858 | 60 | kde-d(10)-kdke | 416 |
| 529878 | 75 | edk-d(10)-kdke | 416 |
| 529898 | 34 | k-(d4)-k-(d4)-k-(d4)-ke | 416 |
| 529566 | 61 | eek-d(10)-kke | 39 |
| 529591 | 71 | kee-d(10)-kke | 39 |
| 529616 | 71 | edk-d(10)-kke | 39 |
| 529641 | 65 | kde-d(10)-kke | 39 |
| 529666 | 70 | kddk-d(9)-kke | 39 |
| 529691 | 67 | kdde-d(9)-kke | 39 |
| 529716 | 75 | eddk-d(9)-kke | 39 |
| 529721 | 71 | ke-d(10)-keke | 39 |
| 529741 | 81 | ek-d(10)-keke | 39 |
| 529761 | 66 | ke-d(10)-kdke | 39 |
| 529781 | 65 | ek-d(10)-kdke | 39 |
| 529801 | 71 | k-d(10)-kekee | 39 |
| 529820 | 74 | k-d(10)-kdkee | 39 |
| 529850 | 63 | kde-d(10)-kdke | 417 |
| 529870 | 72 | edk-d(10)-kdke | 417 |
| 529890 | 23 | k-(d4)-k-(d4)-k-(d4)-ke | 417 |
| 529918 | 54 | eeee-d(9)-kke | 39 |
| 529567 | 75 | eek-d(10)-kke | 262 |
| 529592 | 80 | kee-d(10)-kke | 262 |
| 529617 | 65 | edk-d(10)-kke | 262 |
| 529642 | 62 | kde-d(10)-kke | 262 |
| 529667 | 75 | kddk-d(9)-kke | 262 |
| 529692 | 53 | kdde-d(9)-kke | 262 |
| 529717 | 69 | eddk-d(9)-kke | 262 |
| 529722 | 74 | ke-d(10)-keke | 262 |
| 529742 | 81 | ek-d(10)-keke | 262 |
| 529762 | 66 | ke-d(10)-kdke | 262 |
| 529782 | 68 | ek-d(10)-kdke | 262 |
| 529851 | 68 | kde-d(10)-kdke | 418 |
| 529871 | 77 | edk-d(10)-kdke | 418 |
| 529891 | 36 | k-(d4)-k-(d4)-k-(d4)-ke | 418 |
| 529910 | 60 | eeee-d(9)-kke | 262 |
| 529568 | 79 | eek-d(10)-kke | 263 |
| 529593 | 70 | kee-d(10)-kke | 263 |
| 529618 | 77 | edk-d(10)-kke | 263 |
| 529643 | 72 | kde-d(10)-kke | 263 |
| 529668 | 73 | kddk-d(9)-kke | 263 |
| 529693 | 62 | kdde-d(9)-kke | 263 |
| 529718 | 69 | eddk-d(9)-kke | 263 |
| 529911 | 66 | eeee-d(9)-kke | 263 |
| 529462 | 76 | eek-d(10)-keke | 419 |
| 529268 | 18 | eek-d(10)-keke | 420 |
| 529187 | 46 | eek-d(10)-keke | 421 |
| 529224 | 48 | eek-d(10)-kkke | 421 |
| 529130 | 34 | eee-d(10)-kkk | 422 |
| 529150 | 51 | kkk-d(10)-eee | 422 |
| 529549 | 85 | eek-d(10)-kke | 42 |
| 529574 | 81 | kee-d(10)-kke | 42 |
| 529599 | 64 | edk-d(10)-kke | 42 |
| 529624 | 68 | kde-d(10)-kke | 42 |
| 529649 | 77 | kddk-d(9)-kke | 42 |
| 529674 | 65 | kdde-d(9)-kke | 42 |
| 529699 | 63 | eddk-d(9)-kke | 42 |
| 529931 | 59 | eeee-d(9)-kke | 42 |
| 529810 | 80 | k-d(10)-kekee | 423 |
| 529829 | 67 | k-d(10)-kdkee | 423 |
| 529269 | 65 | eek-d(10)-keke | 424 |
| 529731 | 66 | ke-d(10)-keke | 425 |
| 529751 | 76 | ek-d(10)-keke | 425 |
| 529771 | 73 | ke-d(10)-kdke | 425 |
| 529791 | 65 | ek-d(10)-kdke | 425 |
| 529860 | 73 | kde-d(10)-kdke | 424 |
| 529880 | 74 | edk-d(10)-kdke | 424 |
| 529900 | 62 | k-(d4)-k-(d4)-k-(d4)-ke | 424 |
| 529270 | 69 | eek-d(10)-keke | 480 |
| 529550 | 81 | eek-d(10)-kke | 44 |
| 529575 | 88 | kee-d(10)-kke | 44 |
| 529600 | 78 | edk-d(10)-kke | 44 |
| 529625 | 74 | kde-d(10)-kke | 44 |
| 529650 | 81 | kddk-d(9)-kke | 44 |
| 529675 | 76 | kdde-d(9)-kke | 44 |
| 529700 | 73 | eddk-d(9)-kke | 44 |
| 529920 | 67 | eeee-d(9)-kke | 44 |
| 529271 | 43 | eek-d(10)-keke | 427 |
| 529272 | 0 | eek-d(10)-keke | 428 |
| 529273 | 62 | eek-d(10)-keke | 429 |
| 529274 | 78 | eek-d(10)-keke | 430 |
| 529275 | 70 | eek-d(10)-keke | 431 |
| 529276 | 73 | eek-d(10)-keke | 432 |
| 529277 | 71 | eek-d(10)-keke | 433 |
| 529278 | 72 | eek-d(10)-keke | 434 |
| 529279 | 10 | eek-d(10)-keke | 435 |
| 529280 | 11 | eek-d(10)-keke | 436 |
| 529281 | 82 | eek-d(10)-keke | 437 |
| 529282 | 87 | eek-d(10)-keke | 438 |
| 529803 | 71 | k-d(10)-kekee | 250 |
| 529822 | 72 | k-d(10)-kdkee | 250 |
| 529724 | 76 | ke-d(10)-keke | 439 |
| 529744 | 81 | ek-d(10)-keke | 439 |
| 529764 | 65 | ke-d(10)-kdke | 439 |
| 529784 | 68 | ek-d(10)-kdke | 439 |
| 529853 | 64 | kde-d(10)-kdke | 440 |
| 529873 | 69 | edk-d(10)-kdke | 440 |
| 529893 | 45 | k-(d4)-k-(d4)-k-(d4)-ke | 440 |
| 529937 | 81 | eek-d(10)-keke | 440 |
| 529551 | 88 | eek-d(10)-kke | 48 |
| 529576 | 71 | kee-d(10)-kke | 48 |
| 529601 | 74 | edk-d(10)-kke | 48 |
| 529626 | 72 | kde-d(10)-kke | 48 |
| 529651 | 85 | kddk-d(9)-kke | 48 |
| 529676 | 67 | kdde-d(9)-kke | 48 |
| 529701 | 82 | eddk-d(9)-kke | 48 |
| 529913 | 76 | eeee-d(9)-kke | 48 |
| 529811 | 56 | k-d(10)-kekee | 441 |
| 529830 | 46 | k-d(10)-kdkee | 441 |
| 529732 | 63 | ke-d(10)-keke | 442 |
| 529752 | 72 | ek-d(10)-keke | 442 |
| 529772 | 61 | ke-d(10)-kdke | 442 |
| 529792 | 68 | ek-d(10)-kdke | 442 |
| 529861 | 54 | kde-d(10)-kdke | 443 |
| 529881 | 78 | edk-d(10)-kdke | 443 |
| 529901 | 29 | k-(d4)-k-(d4)-k-(d4)-ke | 443 |
| 529939 | 67 | eek-d(10)-keke | 443 |
| 529283 | 70 | eek-d(10)-keke | 444 |
| 529552 | 72 | eek-d(10)-kke | 49 |
| 529577 | 80 | kee-d(10)-kke | 49 |
| 529602 | 64 | edk-d(10)-kke | 49 |
| 529627 | 56 | kde-d(10)-kke | 49 |
| 529652 | 57 | kddk-d(9)-kke | 49 |
| 529677 | 43 | kdde-d(9)-kke | 49 |
| 529702 | 54 | eddk-d(9)-kke | 49 |
| 529921 | 42 | eeee-d(9)-kke | 49 |
| 529284 | 76 | eek-d(10)-keke | 445 |
| 529285 | 77 | eek-d(10)-keke | 446 |
| 529286 | 68 | eek-d(10)-keke | 447 |
| 529287 | 65 | eek-d(10)-keke | 448 |
| 529719 | 73 | ke-d(10)-keke | 264 |
| 529739 | 83 | ek-d(10)-keke | 264 |
| 529759 | 63 | ke-d(10)-kdke | 264 |
| 529779 | 70 | ek-d(10)-kdke | 244 |
| 529848 | 60 | kde-d(10)-kdke | 449 |
| 529868 | 63 | edk-d(10)-kdke | 449 |
| 529888 | 53 | k-(d4)-k-(d4)-k-(d4)-ke | 449 |
| 529553 | 81 | eek-d(10)-kke | 265 |
| 529578 | 65 | kee-d(10)-kke | 265 |
| 529603 | 60 | edk-d(10)-kke | 265 |
| 529628 | 59 | kde-d(10)-kke | 265 |
| 529653 | 76 | kddk-d(9)-kke | 265 |
| 529678 | 56 | kdde-d(9)-kke | 265 |
| 529703 | 68 | eddk-d(9)-kke | 265 |
| 529908 | 69 | eeee-d(9)-kke | 265 |
| 529168 | 64 | eek-d(10)-keke | 450 |
| 529205 | 62 | eek-d(10)-kkke | 450 |
| 529290 | 53 | eek-d(10)-keke | 451 |
| 529802 | 57 | k-d(10)-kekee | 452 |
| 529821 | 61 | k-d(10)-kdkee | 452 |
| 529292 | 74 | eek-d(10)-keke | 453 |
| 529723 | 68 | ke-d(10)-keke | 454 |
| 529743 | 84 | ek-d(10)-keke | 454 |
| 529763 | 64 | ke-d(10)-kdke | 454 |
| 529783 | 72 | ek-d(10)-kdke | 454 |
| 529852 | 66 | kde-d(10)-kdke | 453 |
| 529872 | 62 | edk-d(10)-kdke | 453 |
| 529892 | 43 | k-(d4)-k-(d4)-k-(d4)-ke | 453 |
| 529554 | 80 | eek-d(10)-kke | 252 |
| 529579 | 83 | kee-d(10)-kke | 252 |
| 529604 | 73 | edk-d(10)-kke | 252 |
| 529629 | 64 | kde-d(10)-kke | 252 |
| 529654 | 69 | kddk-d(9)-kke | 252 |
| 529679 | 52 | kdde-d(9)-kke | 252 |
| 529704 | 63 | eddk-d(9)-kke | 252 |
| 529912 | 64 | eeee-d(9)-kke | 252 |
| 529294 | 74 | eek-d(10)-keke | 455 |
| 529296 | 52 | eek-d(10)-keke | 456 |
| 529298 | 60 | eek-d(10)-keke | 457 |
| 529300 | 71 | eek-d(10)-keke | 458 |
| 529188 | 79 | eek-d(10)-keke | 459 |
| 529225 | 78 | eek-d(10)-kkke | 459 |
| 529131 | 58 | eee-d(10)-kkk | 460 |
| 529151 | 71 | kkk-d(10)-eee | 460 |
| 529302 | 74 | eek-d(10)-keke | 461 |
| 529189 | 64 | eek-d(10)-keke | 222 |
| 529226 | 50 | eek-d(10)-kkke | 222 |
| 529132 | 78 | eee-d(10)-kkk | 462 |
| 529152 | 62 | kkk-d(10)-eee | 462 |
| 529190 | 76 | eek-d(10)-keke | 223 |
| 529227 | 88 | eek-d(10)-kkke | 250 |
| 529133 | 81 | eee-d(10)-kkk | 463 |
| 529153 | 68 | kkk-d(10)-eee | 463 |
| 529191 | 78 | eek-d(10)-keke | 224 |
| 529228 | 85 | eek-d(10)-kkke | 224 |
| 529134 | 75 | eee-d(10)-kkk | 464 |
| 529154 | 61 | kkk-d(10)-eee | 464 |
| 529304 | 89 | eek-d(10)-keke | 465 |
| 529306 | 84 | eek-d(10)-keke | 466 |
| 529308 | 68 | eek-d(10)-keke | 467 |
| 529310 | 59 | eek-d(10)-keke | 468 |
| 529169 | 79 | eek-d(10)-keke | 469 |
| 529206 | 82 | eek-d(10)-kkke | 469 |
| 529312 | 68 | eek-d(10)-keke | 470 |
| 529314 | 61 | eek-d(10)-keke | 471 |
| 529316 | 62 | eek-d(10)-keke | 472 |
| 529555 | 78 | eek-d(10)-kke | 59 |
| 529580 | 73 | kee-d(10)-kke | 59 |
| 529605 | 71 | edk-d(10)-kke | 59 |
| 529630 | 64 | kde-d(10)-kke | 59 |
| 529655 | 63 | kddk-d(9)-kke | 59 |
| 529680 | 43 | kdde-d(9)-kke | 59 |
| 529705 | 63 | eddk-d(9)-kke | 59 |
| 529932 | 60 | eeee-d(9)-kke | 59 |
| 529318 | 82 | eek-d(10)-keke | 473 |
| 529170 | 85 | eek-d(10)-keke | 474 |
| 529207 | 88 | eek-d(10)-kkke | 474 |
| 529171 | 81 | eek-d(10)-keke | 475 |
| 529208 | 84 | eek-d(10)-kkke | 475 |
| 529805 | 40 | k-d(10)-kekee | 476 |
| 529824 | 32 | k-d(10)-kdkee | 476 |
| 529320 | 74 | eek-d(10)-keke | 477 |
| 529726 | 80 | ke-d(10)-keke | 478 |
| 529746 | 82 | ek-d(10)-keke | 478 |
| 529766 | 63 | ke-d(10)-kdke | 478 |
| 529786 | 69 | ek-d(10)-kdke | 478 |
| 529855 | 39 | kde-d(10)-kdke | 477 |
| 529875 | 40 | edk-d(10)-kdke | 477 |
| 529895 | 27 | k-(d4)-k-(d4)-k-(d4)-ke | 477 |
| 529556 | 72 | eek-d(10)-kke | 61 |
| 529581 | 68 | kee-d(10)-kke | 61 |
| 529606 | 54 | edk-d(10)-kke | 61 |
| 529631 | 29 | kde-d(10)-kke | 61 |
| 529656 | 74 | kddk-d(9)-kke | 61 |
| 529681 | 32 | kdde-d(9)-kke | 61 |
| 529706 | 41 | eddk-d(9)-kke | 61 |
| 529915 | 51 | eeee-d(9)-kke | 61 |
| 529172 | 88 | eek-d(10)-keke | 226 |
| 529209 | 87 | eek-d(10)-kkke | 226 |
| 529173 | 92 | eek-d(10)-keke | 227 |
| 529210 | 89 | eek-d(10)-kkke | 227 |
| 529183 | 85 | eek-d(10)-keke | 479 |
| 529220 | 92 | eek-d(10)-kkke | 479 |
| 529126 | 83 | eee-d(10)-kkk | 257 |
| 529146 | 84 | kkk-d(10)-eee | 257 |
| 529174 | 85 | eek-d(10)-keke | 480 |
| 529211 | 86 | eek-d(10)-kkke | 480 |
| 529322 | 71 | eek-d(10)-keke | 481 |
| 529324 | 79 | eek-d(10)-keke | 482 |
| 529326 | 85 | eek-d(10)-keke | 483 |
| 529175 | 92 | eek-d(10)-keke | 228 |
| 529212 | 92 | eek-d(10)-kkke | 228 |
| 529176 | 89 | eek-d(10)-keke | 229 |
| 529213 | 90 | eek-d(10)-kkke | 229 |
| 529804 | 89 | k-d(10)-kekee | 259 |
| 529823 | 89 | k-d(10)-kdkee | 259 |
| 529166 | 83 | eek-d(10)-keke | 230 |
| 529203 | 86 | eek-d(10)-kkke | 230 |
| 529725 | 92 | ke-d(10)-keke | 260 |
| 529745 | 91 | ek-d(10)-keke | 260 |
| 529765 | 88 | ke-d(10)-kdke | 260 |
| 529785 | 91 | ek-d(10)-kdke | 260 |
| 529799 | 89 | k-d(10)-kekee | 260 |
| 529818 | 88 | k-d(10)-kdkee | 260 |
| 529854 | 90 | kde-d(10)-kdke | 230 |
| 529874 | 81 | edk-d(10)-kdke | 230 |
| 529894 | 60 | k-(d4)-k-(d4)-k-(d4)-ke | 230 |
| 529167 | 71 | eek-d(10)-keke | 231 |
| 529204 | 70 | eek-d(10)-kkke | 231 |
| 529557 | 86 | eek-d(10)-kke | 69 |
| 529582 | 86 | kee-d(10)-kke | 69 |
| 529607 | 84 | edk-d(10)-kke | 69 |
| 529632 | 81 | kde-d(10)-kke | 69 |
| 529657 | 85 | kddk-d(9)-kke | 69 |
| 529682 | 78 | kdde-d(9)-kke | 69 |
| 529707 | 79 | eddk-d(9)-kke | 69 |
| 529720 | 75 | ke-d(10)-keke | 69 |
| 529740 | 70 | ek-d(10)-keke | 69 |
| 529760 | 78 | ke-d(10)-kdke | 69 |
| 529780 | 83 | ek-d(10)-kdke | 69 |
| 529849 | 80 | kde-d(10)-kdke | 231 |
| 529869 | 72 | edk-d(10)-kdke | 231 |
| 529889 | 49 | k-(d4)-k-(d4)-k-(d4)-ke | 231 |
| 529914 | 69 | eeee-d(9)-kke | 69 |
| 529328 | 68 | eek-d(10)-keke | 484 |
| 529558 | 71 | eek-d(10)-kke | 71 |
| 529583 | 81 | kee-d(10)-kke | 71 |
| 529608 | 68 | edk-d(10)-kke | 71 |
| 529633 | 73 | kde-d(10)-kke | 71 |
| 529658 | 63 | kddk-d(9)-kke | 71 |
| 529683 | 74 | kdde-d(9)-kke | 71 |
| 529708 | 70 | eddk-d(9)-kke | 71 |
| 529909 | 59 | eeee-d(9)-kke | 71 |
| 529192 | 51 | eek-d(10)-keke | 485 |
| 529229 | 69 | eek-d(10)-kkke | 485 |
| 529135 | 54 | eee-d(10)-kkk | 486 |
| 529155 | 56 | kkk-d(10)-eee | 486 |
| 529330 | 37 | eek-d(10)-keke | 487 |

e = 2′ -MOE, k = cEt, d = 2′-deoxyribonucleoside

### Example 20: Design of Modified Oligonucleotides Comprising 2′-O-Methoxyethyl (2′-MOE) or 6′-(S)—CH₃ Bicyclic Nucleoside (e.g cEt) Modifications

Based on the activity of the antisense oligonucleotides listed above, additional antisense oligonucleotides were designed targeting a Target-X nucleic acid targeting start positions 1147, 1154 or 12842 of Target-X.

The newly designed chimeric antisense oligonucleotides are 16 or 17 nucleotides in length and their motifs are described in Table 35. The chemistry column of Table 35 presents the sugar motif of each oligonucleotide, wherein “e” indicates a 2′-O-methoxyethyl (2′-MOE) nucleoside, “k” indicates a 6′-(S)—CH₃ bicyclic nucleoside (e.g cEt) and “d” indicates a 2′-deoxyribonucleoside. The internucleoside linkages throughout each gapmer are phosphorothioate (P═S) linkages. All cytosine residues throughout each oligonucleotide are 5-methylcytosine.

Each gapmer listed in Table 35 is targeted to the human Target-X genomic sequence.

TABLE 35
Chimeric antisense oligonucleotides targeted to Target-X

| ISIS No | Chemistry | SEQ CODE |
|---|---|---|
| 529544 | eek-d(10)-kke | 21 |
| 529569 | kee-d(10)-kke | 21 |
| 529594 | edk-d(10)-kke | 21 |
| 529619 | kde-d(10)-kke | 21 |
| 529644 | kddk-d(9)-kke | 21 |
| 529669 | kdde-d(9)-kke | 21 |
| 529694 | eddk-d(9)-kke | 21 |
| 529929 | eeee-d(9)-kke | 21 |
| 529809 | k-d(10)-kekee | 488 |
| 529828 | k-d(10)-kdkee | 488 |
| 529730 | ke-d(10)-keke | 489 |
| 529750 | ek-d(10)-keke | 489 |
| 529770 | ke-d(10)-kdke | 489 |
| 529790 | ek-d(10)-kdke | 489 |
| 529859 | kde-d(10)-kdke | 490 |
| 529879 | edk-d(10)-kdke | 490 |
| 529899 | k-d(4)-k-d(4)-k-d(4)-ke | 490 |
| 529545 | eek-d(10)-kke | 22 |
| 529570 | kee-d(10)-kke | 22 |
| 529595 | edk-d(10)-kke | 22 |
| 529620 | kde-d(10)-kke | 22 |
| 529645 | kddk-d(9)-kke | 22 |
| 529670 | kdde-d(9)-kke | 22 |
| 529695 | eddk-d(9)-kke | 22 |
| 529919 | eeee-d(9)-kke | 22 |
| 529548 | eek-d(10)-kke | 41 |
| 529573 | kee-d(10)-kke | 41 |
| 529598 | edk-d(10)-kke | 41 |
| 529623 | kde-d(10)-kke | 41 |
| 529648 | kddk-d(9)-kke | 41 |
| 529673 | kdde-d(9)-kke | 41 |
| 529698 | eddk-d(9)-kke | 41 |
| 529930 | eeee-d(9)-kke | 41 |

e = 2′-MOE, k = cEt, d = 2′-deoxyribonucleoside

### Example 21: Modified Oligonucleotides Comprising 2′-O-Methoxyethyl (2′-MOE) and 6′-(S)—CH₃ Bicyclic Nucleoside (e.g cEt) Modifications Targeting Human Target-X

Additional antisense oligonucleotides were designed targeting a Target-X nucleic acid and were tested for their effects on Target-X mRNA in vitro. ISIS 472998 and ISIS 515554, described in the Examples above were also included in the screen.

The newly designed chimeric antisense oligonucleotides are 16 nucleotides in length and their motifs are described in Table 36. The chemistry column of Table 36 presents the sugar motif of each oligonucleotide, wherein “e” indicates a 2′-O-methoxyethyl (2′-MOE) nucleoside, “k” indicates a 6′-(S)—CH₃ bicyclic nucleoside (e.g cEt) and “d” indicates a 2′-deoxyribonucleoside. The internucleoside linkages throughout each gapmer are phosphorothioate (P═S) linkages. All cytosine residues throughout each oligonucleotide are 5-methylcytosine.

Each gapmer listed in Table 36 is targeted to the human Target-X genomic sequence.

Cultured Hep3B cells at a density of 20,000 cells per well were transfected using electroporation with 2,000 nM antisense oligonucleotide. After a treatment period of approximately 24 hours, RNA was isolated from the cells and Target-X mRNA levels were measured by quantitative real-time PCR. Human primer probe set RTS2927 was used to measure mRNA levels. Target-X mRNA levels were adjusted according to total RNA content, as measured by RIBOGREEN®. Results are presented as percent inhibition of Target-X, relative to untreated control cells.

TABLE 36
Inhibition of human Target-X mRNA levels by chimeric
antisense oligonucleotides targeted to Target-X

| ISIS No | % inhibition | Chemistry | SEQ CODE |
|---|---|---|---|
| 472998 | 88 | kk-d(10)-kk | 74 |
| 515554 | 75 | eee-d(10)-kkk | 493 |
| 534530 | 92 | keke-d(9)-kek | 491 |
| 534563 | 92 | kek-d(9)-ekek | 491 |
| 534596 | 88 | ekee-d(9)-kke | 491 |
| 534629 | 89 | eke-d(9)-ekke | 491 |
| 534662 | 87 | eekk-d(9)-eke | 491 |
| 534695 | 92 | eek-d(9)-keke | 491 |
| 534732 | 90 | ekek-d(8)-keke | 491 |
| 534767 | 92 | keek-d(8)-keek | 491 |
| 534802 | 93 | ekk-d(10)-kke | 491 |
| 534832 | 83 | edk-d(10)-kke | 491 |
| 534862 | 72 | kde-d(10)-kke | 491 |
| 534892 | 82 | eek-d(10)-kke | 491 |
| 534922 | 80 | kddk-d(9)-kke | 491 |
| 534952 | 72 | kdde-d(9)-kke | 491 |
| 534982 | 77 | eddk-d(9)-kke | 491 |
| 535012 | 70 | eeee-d(9)-kke | 491 |
| 535045 | 84 | eeee-d(9)-kkk | 491 |
| 535078 | 87 | eeek-d(9)-kke | 491 |
| 535111 | 63 | eeeee-d(8)-kke | 491 |
| 535144 | 69 | ededk-d(8)-kke | 491 |
| 535177 | 68 | edkde-d(8)-kke | 491 |
| 534531 | 61 | keke-d(9)-kek | 492 |
| 534564 | 30 | kek-d(9)-ekek | 492 |
| 534597 | 67 | ekee-d(9)-kke | 492 |
| 534630 | 54 | eke-d(9)-ekke | 492 |
| 534663 | 94 | eekk-d(9)-eke | 492 |
| 534696 | 68 | eek-d(9)-keke | 492 |
| 534733 | 44 | ekek-d(8)-keke | 492 |
| 534768 | 55 | keek-d(8)-keek | 492 |
| 534803 | 73 | ekk-d(10)-kke | 492 |
| 534833 | 65 | edk-d(10)-kke | 492 |
| 534863 | 53 | kde-d(10)-kke | 492 |
| 534893 | 61 | eek-d(10)-kke | 492 |
| 534923 | 70 | kddk-d(9)-kke | 492 |
| 534953 | 54 | kdde-d(9)-kke | 492 |
| 534983 | 58 | eddk-d(9)-kke | 492 |
| 535013 | 52 | eeee-d(9)-kke | 492 |
| 535046 | 67 | eeee-d(9)-kkk | 492 |
| 535079 | 57 | eeek-d(9)-kke | 492 |
| 535112 | 42 | eeeee-d(8)-kke | 492 |
| 535145 | 41 | ededk-d(8)-kke | 492 |
| 535178 | 35 | edkde-d(8)-kke | 492 |
| 534565 | 87 | kek-d(9)-ekek | 493 |
| 534598 | 72 | ekee-d(9)-kke | 493 |
| 534631 | 70 | eke-d(9)-ekke | 493 |
| 534664 | 94 | eekk-d(9)-eke | 493 |
| 534697 | 90 | eek-d(9)-keke | 493 |
| 534734 | 74 | ekek-d(8)-keke | 493 |
| 534769 | 80 | keek-d(8)-keek | 493 |
| 534804 | 87 | ekk-d(10)-kke | 493 |
| 534834 | 76 | edk-d(10)-kke | 493 |
| 534864 | 56 | kde-d(10)-kke | 493 |
| 534894 | 67 | eek-d(10)-kke | 493 |
| 534924 | 71 | kddk-d(9)-kke | 493 |
| 534954 | 54 | kdde-d(9)-kke | 493 |
| 534984 | 48 | eddk-d(9)-kke | 493 |
| 535014 | 43 | eeee-d(9)-kke | 493 |
| 535047 | 60 | eeee-d(9)-kkk | 493 |
| 535080 | 64 | eeek-d(9)-kke | 493 |
| 535113 | 32 | eeeee-d(8)-kke | 493 |
| 535146 | 31 | ededk-d(8)-kke | 493 |
| 535179 | 28 | edkde-d(8)-kke | 493 |
| 534533 | 82 | keke-d(9)-kek | 494 |
| 534566 | 88 | kek-d(9)-ekek | 494 |
| 534599 | 65 | ekee-d(9)-kke | 494 |
| 534632 | 69 | eke-d(9)-ekke | 494 |
| 534665 | 87 | eekk-d(9)-eke | 494 |
| 534698 | 64 | eek-d(9)-keke | 494 |
| 534735 | 63 | ekek-d(8)-keke | 494 |
| 534770 | 66 | keek-d(8)-keek | 494 |
| 534805 | 87 | ekk-d(10)-kke | 494 |
| 534835 | 68 | edk-d(10)-kke | 494 |
| 534865 | 66 | kde-d(10)-kke | 494 |
| 534895 | 57 | eek-d(10)-kke | 494 |
| 534925 | 82 | kddk-d(9)-kke | 494 |
| 534955 | 76 | kdde-d(9)-kke | 494 |
| 534985 | 71 | eddk-d(9)-kke | 494 |
| 535015 | 59 | eeee-d(9)-kke | 494 |
| 535048 | 69 | eeee-d(9)-kkk | 494 |
| 535081 | 67 | eeek-d(9)-kke | 494 |
| 535114 | 37 | eeeee-d(8)-kke | 494 |
| 535147 | 32 | ededk-d(8)-kke | 494 |
| 535180 | 31 | edkde-d(8)-kke | 494 |
| 534534 | 94 | keke-d(9)-kek | 234 |
| 534567 | 92 | kek-d(9)-ekek | 234 |
| 534600 | 92 | ekee-d(9)-kke | 234 |
| 534633 | 91 | eke-d(9)-ekke | 234 |
| 534666 | 89 | eekk-d(9)-eke | 234 |
| 534699 | 91 | eek-d(9)-keke | 234 |
| 534736 | 83 | ekek-d(8)-keke | 234 |
| 534771 | 80 | keek-d(8)-keek | 234 |
| 534806 | 96 | ekk-d(10)-kke | 234 |
| 534836 | 86 | edk-d(10)-kke | 234 |
| 534866 | 82 | kde-d(10)-kke | 234 |
| 534896 | 82 | eek-d(10)-kke | 234 |
| 534926 | 89 | kddk-d(9)-kke | 234 |
| 534956 | 91 | kdde-d(9)-kke | 234 |
| 534986 | 87 | eddk-d(9)-kke | 234 |
| 535016 | 83 | eeee-d(9)-kke | 234 |
| 535049 | 87 | eeee-d(9)-kkk | 234 |
| 535082 | 87 | eeek-d(9)-kke | 234 |
| 535115 | 77 | eeeee-d(8)-kke | 234 |
| 535148 | 73 | ededk-d(8)-kke | 234 |
| 535181 | 68 | edkde-d(8)-kke | 234 |
| 534535 | 66 | keke-d(9)-kek | 236 |
| 534568 | 85 | kek-d(9)-ekek | 236 |
| 534601 | 51 | ekee-d(9)-kke | 236 |
| 534634 | 80 | eke-d(9)-ekke | 236 |
| 534667 | 90 | eekk-d(9)-eke | 236 |
| 534700 | 88 | eek-d(9)-keke | 236 |
| 534737 | 65 | ekek-d(8)-keke | 236 |
| 534772 | 77 | keek-d(8)-keek | 236 |
| 534807 | 84 | ekk-d(10)-kke | 236 |
| 534837 | 78 | edk-d(10)-kke | 236 |
| 534867 | 44 | kde-d(10)-kke | 236 |
| 534897 | 82 | eek-d(10)-kke | 236 |
| 534927 | 61 | kddk-d(9)-kke | 236 |
| 534957 | 58 | kdde-d(9)-kke | 236 |
| 534987 | 49 | eddk-d(9)-kke | 236 |
| 535017 | 38 | eeee-d(9)-kke | 236 |
| 535050 | 32 | eeee-d(9)-kkk | 236 |
| 535083 | 43 | eeek-d(9)-kke | 236 |
| 535116 | 9 | eeeee-d(8)-kke | 236 |
| 535149 | 23 | ededk-d(8)-kke | 236 |
| 535182 | 18 | edkde-d(8)-kke | 236 |
| 534536 | 89 | keke-d(9)-kek | 238 |
| 534569 | 90 | kek-d(9)-ekek | 238 |
| 534602 | 85 | ekee-d(9)-kke | 238 |
| 534635 | 87 | eke-d(9)-ekke | 238 |
| 534668 | 90 | eekk-d(9)-eke | 238 |
| 534701 | 92 | eek-d(9)-keke | 238 |
| 534738 | 81 | ekek-d(8)-keke | 238 |
| 534773 | 79 | keek-d(8)-keek | 238 |
| 534808 | 90 | ekk-d(10)-kke | 238 |
| 534838 | 88 | edk-d(10)-kke | 238 |
| 534868 | 67 | kde-d(10)-kke | 238 |
| 534898 | 89 | eek-d(10)-kke | 238 |
| 534928 | 81 | kddk-d(9)-kke | 238 |
| 534958 | 78 | kdde-d(9)-kke | 238 |
| 534988 | 66 | eddk-d(9)-kke | 238 |
| 535018 | 78 | eeee-d(9)-kke | 238 |
| 535051 | 76 | eeee-d(9)-kkk | 238 |
| 535084 | 80 | eeek-d(9)-kke | 238 |
| 535117 | 58 | eeeee-d(8)-kke | 238 |
| 535150 | 51 | ededk-d(8)-kke | 238 |
| 535183 | 53 | edkde-d(8)-kke | 238 |
| 534537 | 91 | keke-d(9)-kek | 239 |
| 534570 | 85 | kek-d(9)-ekek | 239 |
| 534603 | 79 | ekee-d(9)-kke | 239 |
| 534636 | 72 | eke-d(9)-ekke | 239 |
| 534669 | 85 | eekk-d(9)-eke | 239 |
| 534702 | 85 | eek-d(9)-keke | 239 |
| 534739 | 73 | ekek-d(8)-keke | 239 |
| 534774 | 77 | keek-d(8)-keek | 239 |
| 534809 | 91 | ekk-d(10)-kke | 239 |
| 534839 | 86 | edk-d(10)-kke | 239 |
| 534869 | 71 | kde-d(10)-kke | 239 |
| 534899 | 82 | eek-d(10)-kke | 239 |
| 534929 | 83 | kddk-d(9)-kke | 239 |
| 534959 | 80 | kdde-d(9)-kke | 239 |
| 534989 | 79 | eddk-d(9)-kke | 239 |
| 535019 | 76 | eeee-d(9)-kke | 239 |
| 535052 | 79 | eeee-d(9)-kkk | 239 |
| 535085 | 81 | eeek-d(9)-kke | 239 |
| 535118 | 58 | eeeee-d(8)-kke | 239 |
| 535151 | 65 | ededk-d(8)-kke | 239 |
| 535184 | 60 | edkde-d(8)-kke | 239 |
| 534516 | 77 | keke-d(9)-kek | 495 |
| 534549 | 80 | kek-d(9)-ekek | 495 |
| 534582 | 73 | ekee-d(9)-kke | 495 |
| 534615 | 79 | eke-d(9)-ekke | 495 |
| 534648 | 67 | eekk-d(9)-eke | 495 |
| 534681 | 87 | eek-d(9)-keke | 495 |
| 534718 | 46 | ekek-d(8)-keke | 495 |
| 534753 | 68 | keek-d(8)-keek | 495 |
| 534788 | 84 | ekk-d(10)-kke | 495 |
| 534818 | 82 | edk-d(10)-kke | 495 |
| 534848 | 75 | kde-d(10)-kke | 495 |
| 534878 | 72 | eek-d(10)-kke | 495 |
| 534908 | 81 | kddk-d(9)-kke | 495 |
| 534938 | 69 | kdde-d(9)-kke | 495 |
| 534968 | 77 | eddk-d(9)-kke | 495 |
| 534998 | 76 | eeee-d(9)-kke | 495 |
| 535031 | 76 | eeee-d(9)-kkk | 495 |
| 535064 | 70 | eeek-d(9)-kke | 495 |
| 535097 | 57 | eeeee-d(8)-kke | 495 |
| 535130 | 69 | ededk-d(8)-kke | 495 |
| 535163 | 58 | edkde-d(8)-kke | 495 |
| 534538 | 71 | keke-d(9)-kek | 241 |
| 534571 | 64 | kek-d(9)-ekek | 241 |
| 534604 | 66 | ekee-d(9)-kke | 241 |
| 534637 | 74 | eke-d(9)-ekke | 241 |
| 534670 | 87 | eekk-d(9)-eke | 241 |
| 534703 | 72 | eek-d(9)-keke | 241 |
| 534740 | 56 | ekek-d(8)-keke | 241 |
| 534775 | 53 | keek-d(8)-keek | 241 |
| 534810 | 78 | ekk-d(10)-kke | 241 |
| 534840 | 73 | edk-d(10)-kke | 241 |
| 534870 | 65 | kde-d(10)-kke | 241 |
| 534900 | 69 | eek-d(10)-kke | 241 |
| 534930 | 67 | kddk-d(9)-kke | 241 |
| 534960 | 62 | kdde-d(9)-kke | 241 |
| 534990 | 66 | eddk-d(9)-kke | 241 |
| 535020 | 61 | eeee-d(9)-kke | 241 |
| 535053 | 47 | eeee-d(9)-kkk | 241 |
| 535086 | 61 | eeek-d(9)-kke | 241 |
| 535119 | 49 | eeeee-d(8)-kke | 241 |
| 535152 | 48 | ededk-d(8)-kke | 241 |
| 535185 | 57 | edkde-d(8)-kke | 241 |
| 534539 | 70 | keke-d(9)-kek | 496 |
| 534572 | 82 | kek-d(9)-ekek | 496 |
| 534605 | 59 | ekee-d(9)-kke | 496 |
| 534638 | 69 | eke-d(9)-ekke | 496 |
| 534671 | 89 | eekk-d(9)-eke | 496 |
| 534704 | 83 | eek-d(9)-keke | 496 |
| 534741 | 47 | ekek-d(8)-keke | 496 |
| 534776 | 46 | keek-d(8)-keek | 496 |
| 534811 | 71 | ekk-d(10)-kke | 496 |
| 534841 | 61 | edk-d(10)-kke | 496 |
| 534871 | 53 | kde-d(10)-kke | 496 |
| 534901 | 55 | eek-d(10)-kke | 496 |
| 534931 | 73 | kddk-d(9)-kke | 496 |
| 534961 | 53 | kdde-d(9)-kke | 496 |
| 534991 | 56 | eddk-d(9)-kke | 496 |
| 535021 | 58 | eeee-d(9)-kke | 496 |
| 535054 | 59 | eeee-d(9)-kkk | 496 |
| 535087 | 0 | eeek-d(9)-kke | 496 |
| 535120 | 41 | eeeee-d(8)-kke | 496 |
| 535153 | 44 | ededk-d(8)-kke | 496 |
| 535186 | 35 | edkde-d(8)-kke | 496 |
| 534573 | 76 | kek-d(9)-ekek | 497 |
| 534606 | 55 | ekee-d(9)-kke | 497 |
| 534639 | 72 | eke-d(9)-ekke | 497 |
| 534672 | 89 | eekk-d(9)-eke | 497 |
| 534705 | 87 | eek-d(9)-keke | 497 |
| 534742 | 84 | ekek-d(8)-keke | 497 |
| 534777 | 79 | keek-d(8)-keek | 497 |
| 534812 | 76 | ekk-d(10)-kke | 497 |
| 534842 | 74 | edk-d(10)-kke | 497 |
| 534872 | 53 | kde-d(10)-kke | 497 |
| 534902 | 70 | eek-d(10)-kke | 497 |
| 534932 | 73 | kddk-d(9)-kke | 497 |
| 534962 | 60 | kdde-d(9)-kke | 497 |
| 534992 | 61 | eddk-d(9)-kke | 497 |
| 535022 | 38 | eeee-d(9)-kke | 497 |
| 535055 | 42 | eeee-d(9)-kkk | 497 |
| 535088 | 56 | eeek-d(9)-kke | 497 |
| 535121 | 5 | eeeee-d(8)-kke | 497 |
| 535154 | 22 | ededk-d(8)-kke | 497 |
| 535187 | 16 | edkde-d(8)-kke | 497 |
| 534541 | 86 | keke-d(9)-kek | 498 |
| 534574 | 89 | kek-d(9)-ekek | 498 |
| 534607 | 59 | ekee-d(9)-kke | 498 |
| 534640 | 76 | eke-d(9)-ekke | 498 |
| 534673 | 89 | eekk-d(9)-eke | 498 |
| 534706 | 86 | eek-d(9)-keke | 498 |
| 534743 | 79 | ekek-d(8)-keke | 498 |
| 534778 | 80 | keek-d(8)-keek | 498 |
| 534813 | 83 | ekk-d(10)-kke | 498 |
| 534843 | 82 | edk-d(10)-kke | 498 |
| 534873 | 83 | kde-d(10)-kke | 498 |
| 534903 | 78 | eek-d(10)-kke | 498 |
| 534933 | 83 | kddk-d(9)-kke | 498 |
| 534963 | 70 | kdde-d(9)-kke | 498 |
| 534993 | 78 | eddk-d(9)-kke | 498 |
| 535023 | 56 | eeee-d(9)-kke | 498 |
| 535056 | 59 | eeee-d(9)-kkk | 498 |
| 535089 | 73 | eeek-d(9)-kke | 498 |
| 535122 | 39 | eeeee-d(8)-kke | 498 |
| 535155 | 60 | ededk-d(8)-kke | 498 |
| 535188 | 41 | edkde-d(8)-kke | 498 |
| 534542 | 75 | keke-d(9)-kek | 499 |
| 534575 | 82 | kek-d(9)-ekek | 499 |
| 534608 | 72 | ekee-d(9)-kke | 499 |
| 534641 | 69 | eke-d(9)-ekke | 499 |
| 534674 | 84 | eekk-d(9)-eke | 499 |
| 534707 | 78 | eek-d(9)-keke | 499 |
| 534744 | 72 | ekek-d(8)-keke | 499 |
| 534779 | 75 | keek-d(8)-keek | 499 |
| 534814 | 81 | ekk-d(10)-kke | 499 |
| 534844 | 75 | edk-d(10)-kke | 499 |
| 534874 | 70 | kde-d(10)-kke | 499 |
| 534904 | 71 | eek-d(10)-kke | 499 |
| 534934 | 73 | kddk-d(9)-kke | 499 |
| 534964 | 72 | kdde-d(9)-kke | 499 |
| 534994 | 69 | eddk-d(9)-kke | 499 |
| 535024 | 56 | eeee-d(9)-kke | 499 |
| 535057 | 63 | eeee-d(9)-kkk | 499 |
| 535090 | 64 | eeek-d(9)-kke | 499 |
| 535123 | 40 | eeeee-d(8)-kke | 499 |
| 535156 | 47 | ededk-d(8)-kke | 499 |
| 535189 | 48 | edkde-d(8)-kke | 499 |
| 534515 | 52 | keke-d(9)-kek | 34 |
| 534548 | 85 | kek-d(9)-ekek | 34 |
| 534581 | 75 | ekee-d(9)-kke | 34 |
| 534614 | 83 | eke-d(9)-ekke | 34 |
| 534647 | 65 | eekk-d(9)-eke | 34 |
| 534680 | 88 | eek-d(9)-keke | 34 |
| 534717 | 76 | ekek-d(8)-keke | 34 |
| 534752 | 79 | keek-d(8)-keek | 34 |
| 534787 | 90 | ekk-d(10)-kke | 34 |
| 535030 | 77 | eeee-d(9)-kkk | 34 |
| 535063 | 75 | eeek-d(9)-kke | 34 |
| 535096 | 54 | eeeee-d(8)-kke | 34 |
| 535129 | 66 | ededk-d(8)-kke | 34 |
| 535162 | 49 | edkde-d(8)-kke | 34 |
| 534543 | 66 | keke-d(9)-kek | 500 |
| 534576 | 69 | kek-d(9)-ekek | 500 |
| 534609 | 77 | ekee-d(9)-kke | 500 |
| 534642 | 62 | eke-d(9)-ekke | 500 |
| 534675 | 80 | eekk-d(9)-eke | 500 |
| 534708 | 81 | eek-d(9)-keke | 500 |
| 534745 | 68 | ekek-d(8)-keke | 500 |
| 534780 | 69 | keek-d(8)-keek | 500 |
| 534815 | 85 | ekk-d(10)-kke | 500 |
| 534845 | 72 | edk-d(10)-kke | 500 |
| 534875 | 56 | kde-d(10)-kke | 500 |
| 534905 | 65 | eek-d(10)-kke | 500 |
| 534935 | 78 | kddk-d(9)-kke | 500 |
| 534965 | 48 | kdde-d(9)-kke | 500 |
| 534995 | 62 | eddk-d(9)-kke | 500 |
| 535025 | 58 | eeee-d(9)-kke | 500 |
| 535058 | 60 | eeee-d(9)-kkk | 500 |
| 535091 | 61 | eeek-d(9)-kke | 500 |
| 535124 | 51 | eeeee-d(8)-kke | 500 |
| 535157 | 55 | ededk-d(8)-kke | 500 |
| 535190 | 47 | edkde-d(8)-kke | 500 |
| 534517 | 71 | keke-d(9)-kek | 501 |
| 534550 | 80 | kek-d(9)-ekek | 501 |
| 534583 | 70 | ekee-d(9)-kke | 501 |
| 534616 | 84 | eke-d(9)-ekke | 501 |
| 534649 | 68 | eekk-d(9)-eke | 501 |
| 534682 | 87 | eek-d(9)-keke | 501 |
| 534719 | 90 | ekek-d(8)-keke | 501 |
| 534754 | 83 | keek-d(8)-keek | 501 |
| 534789 | 86 | ekk-d(10)-kke | 501 |
| 534819 | 69 | edk-d(10)-kke | 501 |
| 534849 | 62 | kde-d(10)-kke | 501 |
| 534879 | 69 | eek-d(10)-kke | 501 |
| 534909 | 73 | kddk-d(9)-kke | 501 |
| 534939 | 49 | kdde-d(9)-kke | 501 |
| 534969 | 47 | eddk-d(9)-kke | 501 |
| 534999 | 51 | eeee-d(9)-kke | 501 |
| 535032 | 51 | eeee-d(9)-kkk | 501 |
| 535065 | 64 | eeek-d(9)-kke | 501 |
| 535098 | 31 | eeeee-d(8)-kke | 501 |
| 535131 | 31 | ededk-d(8)-kke | 501 |
| 535164 | 40 | edkde-d(8)-kke | 501 |
| 534518 | 81 | keke-d(9)-kek | 502 |
| 534551 | 88 | kek-d(9)-ekek | 502 |
| 534584 | 78 | ekee-d(9)-kke | 502 |
| 534617 | 80 | eke-d(9)-ekke | 502 |
| 534650 | 83 | eekk-d(9)-eke | 502 |
| 534683 | 93 | eek-d(9)-keke | 502 |
| 534720 | 87 | ekek-d(8)-keke | 502 |
| 534755 | 82 | keek-d(8)-keek | 502 |
| 534790 | 89 | ekk-d(10)-kke | 502 |
| 534820 | 64 | edk-d(10)-kke | 502 |
| 534850 | 38 | kde-d(10)-kke | 502 |
| 534880 | 68 | eek-d(10)-kke | 502 |
| 534910 | 60 | kddk-d(9)-kke | 502 |
| 534940 | 37 | kdde-d(9)-kke | 502 |
| 534970 | 59 | eddk-d(9)-kke | 502 |
| 535000 | 30 | eeee-d(9)-kke | 502 |
| 535033 | 44 | eeee-d(9)-kkk | 502 |
| 535066 | 64 | eeek-d(9)-kke | 502 |
| 535099 | 22 | eeeee-d(8)-kke | 502 |
| 535132 | 54 | ededk-d(8)-kke | 502 |
| 535165 | 45 | edkde-d(8)-kke | 502 |
| 534544 | 80 | keke-d(9)-kek | 503 |
| 534577 | 83 | kek-d(9)-ekek | 503 |
| 534610 | 62 | ekee-d(9)-kke | 503 |
| 534643 | 66 | eke-d(9)-ekke | 503 |
| 534676 | 95 | eekk-d(9)-eke | 503 |
| 534709 | 86 | eek-d(9)-keke | 503 |
| 534746 | 73 | ekek-d(8)-keke | 503 |
| 534781 | 71 | keek-d(8)-keek | 503 |
| 534816 | 83 | ekk-d(10)-kke | 503 |
| 534846 | 73 | edk-d(10)-kke | 503 |
| 534876 | 39 | kde-d(10)-kke | 503 |
| 534906 | 67 | eek-d(10)-kke | 503 |
| 534936 | 66 | kddk-d(9)-kke | 503 |
| 534966 | 48 | kdde-d(9)-kke | 503 |
| 534996 | 56 | eddk-d(9)-kke | 503 |
| 535026 | 39 | eeee-d(9)-kke | 503 |
| 535059 | 45 | eeee-d(9)-kkk | 503 |
| 535092 | 48 | eeek-d(9)-kke | 503 |
| 535125 | 26 | eeeee-d(8)-kke | 503 |
| 535158 | 44 | ededk-d(8)-kke | 503 |
| 535191 | 34 | edkde-d(8)-kke | 503 |
| 534545 | 83 | keke-d(9)-kek | 504 |
| 534578 | 81 | kek-d(9)-ekek | 504 |
| 534611 | 78 | ekee-d(9)-kke | 504 |
| 534644 | 72 | eke-d(9)-ekke | 504 |
| 534677 | 92 | eekk-d(9)-eke | 504 |
| 534710 | 78 | eek-d(9)-keke | 504 |
| 534747 | 85 | ekek-d(8)-keke | 504 |
| 534782 | 85 | keek-d(8)-keek | 504 |
| 534817 | 88 | ekk-d(10)-kke | 504 |
| 534847 | 73 | edk-d(10)-kke | 504 |
| 534877 | 66 | kde-d(10)-kke | 504 |
| 534907 | 73 | eek-d(10)-kke | 504 |
| 534937 | 85 | kddk-d(9)-kke | 504 |
| 534967 | 80 | kdde-d(9)-kke | 504 |
| 534997 | 74 | eddk-d(9)-kke | 504 |
| 535027 | 64 | eeee-d(9)-kke | 504 |
| 535060 | 68 | eeee-d(9)-kkk | 504 |
| 535093 | 73 | eeek-d(9)-kke | 504 |
| 535126 | 42 | eeeee-d(8)-kke | 504 |
| 535159 | 49 | ededk-d(8)-kke | 504 |
| 535192 | 51 | edkde-d(8)-kke | 504 |
| 534519 | 87 | keke-d(9)-kek | 505 |
| 534552 | 85 | kek-d(9)-ekek | 505 |
| 534585 | 76 | ekee-d(9)-kke | 505 |
| 534618 | 78 | eke-d(9)-ekke | 505 |
| 534651 | 79 | eekk-d(9)-eke | 505 |
| 534684 | 87 | eek-d(9)-keke | 505 |
| 534721 | 89 | ekek-d(8)-keke | 505 |
| 534756 | 90 | keek-d(8)-keek | 505 |
| 534791 | 84 | ekk-d(10)-kke | 505 |
| 534821 | 79 | edk-d(10)-kke | 505 |
| 534851 | 64 | kde-d(10)-kke | 505 |
| 534881 | 65 | eek-d(10)-kke | 505 |
| 534911 | 85 | kddk-d(9)-kke | 505 |
| 534941 | 66 | kdde-d(9)-kke | 505 |
| 534971 | 75 | eddk-d(9)-kke | 505 |
| 535001 | 62 | eeee-d(9)-kke | 505 |
| 535034 | 65 | eeee-d(9)-kkk | 505 |
| 535067 | 76 | eeek-d(9)-kke | 505 |
| 535100 | 5 | eeeee-d(8)-kke | 505 |
| 535133 | 30 | ededk-d(8)-kke | 505 |
| 535166 | 23 | edkde-d(8)-kke | 505 |
| 534520 | 87 | keke-d(9)-kek | 251 |
| 534553 | 79 | kek-d(9)-ekek | 251 |
| 534586 | 60 | ekee-d(9)-kke | 251 |
| 534619 | 62 | eke-d(9)-ekke | 251 |
| 534652 | 84 | eekk-d(9)-eke | 251 |
| 534685 | 84 | eek-d(9)-keke | 251 |
| 534722 | 75 | ekek-d(8)-keke | 251 |
| 534757 | 81 | keek-d(8)-keek | 251 |
| 534792 | 87 | ekk-d(10)-kke | 251 |
| 534822 | 80 | edk-d(10)-kke | 251 |
| 534852 | 38 | kde-d(10)-kke | 251 |
| 534882 | 75 | eek-d(10)-kke | 251 |
| 534912 | 74 | kddk-d(9)-kke | 251 |
| 534942 | 58 | kdde-d(9)-kke | 251 |
| 534972 | 59 | eddk-d(9)-kke | 251 |
| 535002 | 50 | eeee-d(9)-kke | 251 |
| 535035 | 57 | eeee-d(9)-kkk | 251 |
| 535068 | 67 | eeek-d(9)-kke | 251 |
| 535101 | 24 | eeeee-d(8)-kke | 251 |
| 535134 | 23 | ededk-d(8)-kke | 251 |
| 535167 | 26 | edkde-d(8)-kke | 251 |
| 534513 | 90 | keke-d(9)-kek | 252 |
| 534546 | 92 | kek-d(9)-ekek | 252 |
| 534579 | 78 | ekee-d(9)-kke | 252 |
| 534612 | 82 | eke-d(9)-ekke | 252 |
| 534645 | 73 | eekk-d(9)-eke | 252 |
| 534678 | 91 | eek-d(9)-keke | 252 |
| 534715 | 87 | ekek-d(8)-keke | 252 |
| 534750 | 88 | keek-d(8)-keek | 252 |
| 534785 | 89 | ekk-d(10)-kke | 252 |
| 535028 | 52 | eeee-d(9)-kkk | 252 |
| 535061 | 73 | eeek-d(9)-kke | 252 |
| 535094 | 61 | eeeee-d(8)-kke | 252 |
| 535127 | 59 | ededk-d(8)-kke | 252 |
| 535160 | 62 | edkde-d(8)-kke | 252 |
| 534521 | 86 | keke-d(9)-kek | 506 |
| 534554 | 87 | kek-d(9)-ekek | 506 |
| 534587 | 62 | ekee-d(9)-kke | 506 |
| 534620 | 68 | eke-d(9)-ekke | 506 |
| 534653 | 77 | eekk-d(9)-eke | 506 |
| 534686 | 90 | eek-d(9)-keke | 506 |
| 534723 | 88 | ekek-d(8)-keke | 506 |
| 534758 | 79 | keek-d(8)-keek | 506 |
| 534793 | 85 | ekk-d(10)-kke | 506 |
| 534823 | 81 | edk-d(10)-kke | 506 |
| 534853 | 59 | kde-d(10)-kke | 506 |
| 534883 | 69 | eek-d(10)-kke | 506 |
| 534913 | 76 | kddk-d(9)-kke | 506 |
| 534943 | 53 | kdde-d(9)-kke | 506 |
| 534973 | 61 | eddk-d(9)-kke | 506 |
| 535003 | 53 | eeee-d(9)-kke | 506 |
| 535036 | 35 | eeee-d(9)-kkk | 506 |
| 535069 | 62 | eeek-d(9)-kke | 506 |
| 535102 | 31 | eeeee-d(8)-kke | 506 |
| 535135 | 44 | ededk-d(8)-kke | 506 |
| 535168 | 34 | edkde-d(8)-kke | 506 |
| 534522 | 83 | keke-d(9)-kek | 507 |
| 534555 | 81 | kek-d(9)-ekek | 507 |
| 534588 | 72 | ekee-d(9)-kke | 507 |
| 534621 | 74 | eke-d(9)-ekke | 507 |
| 534654 | 78 | eekk-d(9)-eke | 507 |
| 534687 | 91 | eek-d(9)-keke | 507 |
| 534724 | 84 | ekek-d(8)-keke | 507 |
| 534759 | 86 | keek-d(8)-keek | 507 |
| 534794 | 78 | ekk-d(10)-kke | 507 |
| 534824 | 75 | edk-d(10)-kke | 507 |
| 534854 | 63 | kde-d(10)-kke | 507 |
| 534884 | 60 | eek-d(10)-kke | 507 |
| 534914 | 75 | kddk-d(9)-kke | 507 |
| 534944 | 69 | kdde-d(9)-kke | 507 |
| 534974 | 66 | eddk-d(9)-kke | 507 |
| 535004 | 56 | eeee-d(9)-kke | 507 |
| 535037 | 50 | eeee-d(9)-kkk | 507 |
| 535070 | 68 | eeek-d(9)-kke | 507 |
| 535103 | 55 | eeeee-d(8)-kke | 507 |
| 535136 | 51 | ededk-d(8)-kke | 507 |
| 535169 | 54 | edkde-d(8)-kke | 507 |
| 534523 | 89 | keke-d(9)-kek | 253 |
| 534556 | 91 | kek-d(9)-ekek | 253 |
| 534589 | 88 | ekee-d(9)-kke | 253 |
| 534622 | 93 | eke-d(9)-ekke | 253 |
| 534655 | 72 | eekk-d(9)-eke | 253 |
| 534688 | 92 | eek-d(9)-keke | 253 |
| 534725 | 87 | ekek-d(8)-keke | 253 |
| 534760 | 92 | keek-d(8)-keek | 253 |
| 534795 | 93 | ekk-d(10)-kke | 253 |
| 534825 | 82 | edk-d(10)-kke | 253 |
| 534855 | 73 | kde-d(10)-kke | 253 |
| 534885 | 82 | eek-d(10)-kke | 253 |
| 534915 | 88 | kddk-d(9)-kke | 253 |
| 534945 | 82 | kdde-d(9)-kke | 253 |
| 534975 | 68 | eddk-d(9)-kke | 253 |
| 535005 | 69 | eeee-d(9)-kke | 253 |
| 535038 | 72 | eeee-d(9)-kkk | 253 |
| 535071 | 74 | eeek-d(9)-kke | 253 |
| 535104 | 61 | eeeee-d(8)-kke | 253 |
| 535137 | 67 | ededk-d(8)-kke | 253 |
| 535170 | 51 | edkde-d(8)-kke | 253 |
| 534524 | 95 | keke-d(9)-kek | 254 |
| 534557 | 98 | kek-d(9)-ekek | 254 |
| 534590 | 91 | ekee-d(9)-kke | 254 |
| 534623 | 91 | eke-d(9)-ekke | 254 |
| 534656 | 90 | eekk-d(9)-eke | 254 |
| 534689 | 92 | eek-d(9)-keke | 254 |
| 534726 | 57 | ekek-d(8)-keke | 254 |
| 534761 | 89 | keek-d(8)-keek | 254 |
| 534796 | 93 | ekk-d(10)-kke | 254 |
| 534826 | 89 | edk-d(10)-kke | 254 |
| 534856 | 87 | kde-d(10)-kke | 254 |
| 534886 | 85 | eek-d(10)-kke | 254 |
| 534916 | 87 | kddk-d(9)-kke | 254 |
| 534946 | 86 | kdde-d(9)-kke | 254 |
| 534976 | 77 | eddk-d(9)-kke | 254 |
| 535006 | 83 | eeee-d(9)-kke | 254 |
| 535039 | 86 | eeee-d(9)-kkk | 254 |
| 535072 | 87 | eeek-d(9)-kke | 254 |
| 535105 | 68 | eeeee-d(8)-kke | 254 |
| 535138 | 70 | ededk-d(8)-kke | 254 |
| 535171 | 65 | edkde-d(8)-kke | 254 |
| 534558 | 92 | kek-d(9)-ekek | 255 |
| 534591 | 91 | ekee-d(9)-kke | 255 |
| 534624 | 86 | eke-d(9)-ekke | 255 |
| 534657 | 90 | eekk-d(9)-eke | 255 |
| 534690 | 76 | eek-d(9)-keke | 255 |
| 534727 | 92 | ekek-d(8)-keke | 255 |
| 534762 | 91 | keek-d(8)-keek | 255 |
| 534797 | 94 | ekk-d(10)-kke | 255 |
| 534827 | 90 | edk-d(10)-kke | 255 |
| 534857 | 80 | kde-d(10)-kke | 255 |
| 534887 | 76 | eek-d(10)-kke | 255 |
| 534917 | 91 | kddk-d(9)-kke | 255 |
| 534947 | 91 | kdde-d(9)-kke | 255 |
| 534977 | 86 | eddk-d(9)-kke | 255 |
| 535007 | 80 | eeee-d(9)-kke | 255 |
| 535040 | 86 | eeee-d(9)-kkk | 255 |
| 535073 | 87 | eeek-d(9)-kke | 255 |
| 535106 | 70 | eeeee-d(8)-kke | 255 |
| 535139 | 73 | ededk-d(8)-kke | 255 |
| 535172 | 69 | edkde-d(8)-kke | 255 |
| 534514 | 90 | keke-d(9)-kek | 61 |
| 534547 | 92 | kek-d(9)-ekek | 61 |
| 534580 | 78 | ekee-d(9)-kke | 61 |
| 534613 | 80 | eke-d(9)-ekke | 61 |
| 534646 | 79 | eekk-d(9)-eke | 61 |
| 534679 | 93 | eek-d(9)-keke | 61 |
| 534716 | 94 | ekek-d(8)-keke | 61 |
| 534751 | 86 | keek-d(8)-keek | 61 |
| 534786 | 83 | ekk-d(10)-kke | 61 |
| 535029 | 45 | eeee-d(9)-kkk | 61 |
| 535062 | 81 | eeek-d(9)-kke | 61 |
| 535095 | 57 | eeeee-d(8)-kke | 61 |
| 535128 | 58 | ededk-d(8)-kke | 61 |
| 535161 | 49 | edkde-d(8)-kke | 61 |
| 534526 | 94 | keke-d(9)-kek | 256 |
| 534559 | 95 | kek-d(9)-ekek | 256 |
| 534592 | 93 | ekee-d(9)-kke | 256 |
| 534625 | 93 | eke-d(9)-ekke | 256 |
| 534658 | 93 | eekk-d(9)-eke | 256 |
| 534691 | 96 | eek-d(9)-keke | 256 |
| 534728 | 93 | ekek-d(8)-keke | 256 |
| 534763 | 93 | keek-d(8)-keek | 256 |
| 534798 | 97 | ekk-d(10)-kke | 256 |
| 534828 | 94 | edk-d(10)-kke | 256 |
| 534858 | 92 | kde-d(10)-kke | 256 |
| 534888 | 93 | eek-d(10)-kke | 256 |
| 534918 | 95 | kddk-d(9)-kke | 256 |
| 534948 | 93 | kdde-d(9)-kke | 256 |
| 534978 | 91 | eddk-d(9)-kke | 256 |
| 535008 | 88 | eeee-d(9)-kke | 256 |
| 535041 | 87 | eeee-d(9)-kkk | 256 |
| 535074 | 90 | eeek-d(9)-kke | 256 |
| 535107 | 78 | eeeee-d(8)-kke | 256 |
| 535140 | 81 | ededk-d(8)-kke | 256 |
| 535173 | 81 | edkde-d(8)-kke | 256 |
| 534527 | 95 | keke-d(9)-kek | 258 |
| 534560 | 96 | kek-d(9)-ekek | 258 |
| 534593 | 87 | ekee-d(9)-kke | 258 |
| 534626 | 85 | eke-d(9)-ekke | 258 |
| 534659 | 90 | eekk-d(9)-eke | 258 |
| 534692 | 91 | eek-d(9)-keke | 258 |
| 534729 | 91 | ekek-d(8)-keke | 258 |
| 534764 | 91 | keek-d(8)-keek | 258 |
| 534799 | 96 | ekk-d(10)-kke | 258 |
| 534829 | 91 | edk-d(10)-kke | 258 |
| 534859 | 87 | kde-d(10)-kke | 258 |
| 534889 | 81 | eek-d(10)-kke | 258 |
| 534919 | 92 | kddk-d(9)-kke | 258 |
| 534949 | 91 | kdde-d(9)-kke | 258 |
| 534979 | 84 | eddk-d(9)-kke | 258 |
| 535009 | 78 | eeee-d(9)-kke | 258 |
| 535042 | 76 | eeee-d(9)-kkk | 258 |
| 535075 | 83 | eeek-d(9)-kke | 258 |
| 535108 | 64 | eeeee-d(8)-kke | 258 |
| 535141 | 69 | ededk-d(8)-kke | 258 |
| 535174 | 65 | edkde-d(8)-kke | 258 |
| 534528 | 94 | keke-d(9)-kek | 260 |
| 534561 | 0 | kek-d(9)-ekek | 260 |
| 534594 | 92 | ekee-d(9)-kke | 260 |
| 534627 | 90 | eke-d(9)-ekke | 260 |
| 534660 | 92 | eekk-d(9)-eke | 260 |
| 534693 | 95 | eek-d(9)-keke | 260 |
| 534730 | 93 | ekek-d(8)-keke | 260 |
| 534765 | 92 | keek-d(8)-keek | 260 |
| 534800 | 93 | ekk-d(10)-kke | 260 |
| 534830 | 93 | edk-d(10)-kke | 260 |
| 534860 | 85 | kde-d(10)-kke | 260 |
| 534890 | 91 | eek-d(10)-kke | 260 |
| 534920 | 93 | kddk-d(9)-kke | 260 |
| 534950 | 90 | kdde-d(9)-kke | 260 |
| 534980 | 88 | eddk-d(9)-kke | 260 |
| 535010 | 88 | eeee-d(9)-kke | 260 |
| 535043 | 89 | eeee-d(9)-kkk | 260 |
| 535076 | 88 | eeek-d(9)-kke | 260 |
| 535109 | 76 | eeeee-d(8)-kke | 260 |
| 535142 | 86 | ededk-d(8)-kke | 260 |
| 535175 | 71 | edkde-d(8)-kke | 260 |
| 534529 | 70 | keke-d(9)-kek | 261 |
| 534562 | 86 | kek-d(9)-ekek | 261 |
| 534595 | 56 | ekee-d(9)-kke | 261 |
| 534628 | 73 | eke-d(9)-ekke | 261 |
| 534661 | 64 | eekk-d(9)-eke | 261 |
| 534694 | 75 | eek-d(9)-keke | 261 |
| 534731 | 47 | ekek-d(8)-keke | 261 |
| 534766 | 30 | keek-d(8)-keek | 261 |
| 534801 | 83 | ekk-d(10)-kke | 261 |
| 534831 | 84 | edk-d(10)-kke | 261 |
| 534861 | 71 | kde-d(10)-kke | 261 |
| 534891 | 73 | eek-d(10)-kke | 261 |
| 534921 | 55 | kddk-d(9)-kke | 261 |
| 534951 | 61 | kdde-d(9)-kke | 261 |
| 534981 | 48 | eddk-d(9)-kke | 261 |
| 535011 | 54 | eeee-d(9)-kke | 261 |
| 535044 | 46 | eeee-d(9)-kkk | 261 |
| 535077 | 29 | eeek-d(9)-kke | 261 |
| 535110 | 19 | eeeee-d(8)-kke | 261 |
| 535143 | 15 | ededk-d(8)-kke | 261 |
| 535176 | 37 | edkde-d(8)-kke | 261 |

e = 2′-MOE, k = cEt, d = 2′-deoxynucleoside

### Example 22: Modified Antisense Oligonucleotides Comprising 2′-O-Methoxyethyl (2′-MOE) and 6′-(S)—CH₃ Bicyclic Nucleoside (e.g cEt) Modifications Targeting Human Target-X Targeting Intronic Repeats

Additional antisense oligonucleotides were designed targeting the intronic repeat regions of Target-X.

The newly designed chimeric antisense oligonucleotides and their motifs are described in Table 37. The internucleoside linkages throughout each gapmer are phosphorothioate linkages (P═S) and are designated as “s”. Nucleosides followed by “d” indicate 2′-deoxyribonucleosides. Nucleosides followed by “k” indicate 6′-(S)—CH₃ bicyclic nucleosides (e.g cEt). Nucleosides followed by “e” indicate 2′-O-methoxyethyl (2′-MOE) nucleosides. “N” indicates modified or naturally occurring nucleobases (A, T, C, G, U, or 5-methyl C).

Each gapmer listed in Table 37 is targeted to the intronic region of human Target-X genomic sequence, designated herein as Target-X.

Cultured Hep3B cells at a density of 20,000 cells per well were transfected using electroporation with 2,000 nM antisense oligonucleotide. After a treatment period of approximately 24 hours, RNA was isolated from the cells and Target-X mRNA levels were measured by quantitative real-time PCR. Human primer probe set was used to measure mRNA levels. Target-X mRNA levels were adjusted according to total RNA content, as measured by RIBOGREEN®. Results are presented as percent inhibition of Target-X, relative to untreated control cells.

TABLE 37
Inhibition of human Target-X mRNA levels by chimeric antisense
oligonucleotides targeted to Target-X

| | | | | SEQ |
|---|---|---|---|---|
| | ISIS | % | SEQ | ID |
| Sequence (5′ to 3′) | No | inhibition | CODE | NO |
| Nks Nks Nds Nds Nds Nds Nds Nds Nds Nds Nds | 472998 | 90 | 508 | 7 |
| Nds Nks Nk | | | | |
| Nks Nks Nks Nds Nds Nds Nds Nds Nds Nds Nds | 473327 | 88 | 30 | 6 |
| Nds Nds Nes Nes Ne | | | | |
| Nes Nes Nes Nds Nds Nds Nds Nds Nds Nds Nds | 537024 | 74 | 509 | 6 |
| Nds Nds Nks Nks Nk | | | | |
| Nes Nes Nes Nds Nds Nds Nds Nds Nds Nds Nds | 537025 | 79 | 510 | 6 |
| Nds Nds Nks Nks Nk | | | | |
| Nes Nes Nes Nds Nds Nds Nds Nds Nds Nds Nds | 537026 | 76 | 511 | 6 |
| Nds Nds Nks Nks Nk | | | | |
| Nes Nes Nes Nds Nds Nds Nds Nds Nds Nds Nds | 537028 | 37 | 512 | 6 |
| Nds Nds Nks Nks Nk | | | | |
| Nes Nes Nes Nds Nds Nds Nds Nds Nds Nds Nds | 537029 | 45 | 513 | 6 |
| Nds Nds Nks Nks Nk | | | | |
| Nes Nes Nes Nds Nds Nds Nds Nds Nds Nds Nds | 537030 | 67 | 514 | 6 |
| Nds Nds Nks Nks Nk | | | | |
| Nes Nes Nes Nds Nds Nds Nds Nds Nds Nds Nds | 537031 | 59 | 515 | 6 |
| Nds Nds Nks Nks Nk | | | | |
| Nes Nes Nes Nds Nds Nds Nds Nds Nds Nds Nds | 537032 | 9 | 516 | 6 |
| Nds Nds Nks Nks Nk | | | | |
| Nes Nes Nes Nds Nds Nds Nds Nds Nds Nds Nds | 537033 | 65 | 517 | 6 |
| Nds Nds Nks Nks Nk | | | | |
| Nes Nes Nes Nds Nds Nds Nds Nds Nds Nds Nds | 537034 | 71 | 518 | 6 |
| Nds Nds Nks Nks Nk | | | | |
| Nes Nes Nes Nds Nds Nds Nds Nds Nds Nds Nds | 537035 | 68 | 519 | 6 |
| Nds Nds Nks Nks Nk | | | | |
| Nes Nes Nes Nds Nds Nds Nds Nds Nds Nds Nds | 537036 | 74 | 520 | 6 |
| Nds Nds Nks Nks Nk | | | | |
| Nes Nes Nes Nds Nds Nds Nds Nds Nds Nds Nds | 537038 | 69 | 521 | 6 |
| Nds Nds Nks Nks Nk | | | | |
| Nes Nes Nes Nds Nds Nds Nds Nds Nds Nds Nds | 537039 | 67 | 522 | 6 |
| Nds Nds Nks Nks Nk | | | | |
| Nes Nes Nes Nds Nds Nds Nds Nds Nds Nds Nds | 537040 | 68 | 523 | 6 |
| Nds Nds Nks Nks Nk | | | | |
| Nes Nes Nes Nds Nds Nds Nds Nds Nds Nds Nds | 537041 | 76 | 524 | 6 |
| Nds Nds Nks Nks Nk | | | | |
| Nes Nes Nes Nds Nds Nds Nds Nds Nds Nds Nds | 537042 | 77 | 525 | 6 |
| Nds Nds Nks Nks Nk | | | | |
| Nes Nes Nes Nds Nds Nds Nds Nds Nds Nds Nds | 537043 | 70 | 526 | 6 |
| Nds Nds Nks Nks Nk | | | | |
| Nes Nes Nes Nds Nds Nds Nds Nds Nds Nds Nds | 537044 | 82 | 527 | 6 |
| Nds Nds Nks Nks Nk | | | | |
| Nes Nes Nes Nds Nds Nds Nds Nds Nds Nds Nds | 537045 | 69 | 528 | 6 |
| Nds Nds Nks Nks Nk | | | | |
| Nes Nes Nes Nds Nds Nds Nds Nds Nds Nds Nds | 537047 | 35 | 529 | 6 |
| Nds Nds Nks Nks Nk | | | | |
| Nes Nes Nes Nds Nds Nds Nds Nds Nds Nds Nds | 537049 | 62 | 530 | 6 |
| Nds Nds Nks Nks Nk | | | | |
| Nes Nes Nes Nds Nds Nds Nds Nds Nds Nds Nds | 537051 | 62 | 531 | 6 |
| Nds Nds Nks Nks Nk | | | | |
| Nes Nes Nes Nds Nds Nds Nds Nds Nds Nds Nds | 537055 | 16 | 532 | 6 |
| Nds Nds Nks Nks Nk | | | | |
| Nes Nes Nes Nds Nds Nds Nds Nds Nds Nds Nds | 537056 | 25 | 533 | 6 |
| Nds Nds Nks Nks Nk | | | | |
| Nes Nes Nes Nds Nds Nds Nds Nds Nds Nds Nds | 537057 | 49 | 534 | 6 |
| Nds Nds Nks Nks Nk | | | | |
| Nes Nes Nes Nds Nds Nds Nds Nds Nds Nds Nds | 537058 | 49 | 535 | 6 |
| Nds Nds Nks Nks Nk | | | | |
| Nes Nes Nes Nds Nds Nds Nds Nds Nds Nds Nds | 537059 | 53 | 536 | 6 |
| Nds Nds Nks Nks Nk | | | | |
| Nes Nes Nes Nds Nds Nds Nds Nds Nds Nds Nds | 537060 | 73 | 537 | 6 |
| Nds Nds Nks Nks Nk | | | | |
| Nes Nes Nes Nds Nds Nds Nds Nds Nds Nds Nds | 537061 | 70 | 538 | 6 |
| Nds Nds Nks Nks Nk | | | | |
| Nes Nes Nes Nds Nds Nds Nds Nds Nds Nds Nds | 537062 | 69 | 539 | 6 |
| Nds Nds Nks Nks Nk | | | | |
| Nes Nes Nes Nds Nds Nds Nds Nds Nds Nds Nds | 537063 | 68 | 540 | 6 |
| Nds Nds Nks Nks Nk | | | | |
| Nes Nes Nes Nds Nds Nds Nds Nds Nds Nds Nds | 537064 | 71 | 541 | 6 |
| Nds Nds Nks Nks Nk | | | | |
| Nes Nes Nes Nds Nds Nds Nds Nds Nds Nds Nds | 537065 | 67 | 542 | 6 |
| Nds Nds Nks Nks Nk | | | | |
| Nes Nes Nes Nds Nds Nds Nds Nds Nds Nds Nds | 537066 | 68 | 543 | 6 |
| Nds Nds Nks Nks Nk | | | | |
| Nes Nes Nes Nds Nds Nds Nds Nds Nds Nds Nds | 537067 | 71 | 544 | 6 |
| Nds Nds Nks Nks Nk | | | | |
| Nes Nes Nes Nds Nds Nds Nds Nds Nds Nds Nds | 537068 | 86 | 545 | 6 |
| Nds Nds Nks Nks Nk | | | | |
| Nes Nes Nes Nds Nds Nds Nds Nds Nds Nds Nds | 537069 | 82 | 546 | 6 |
| Nds Nds Nks Nks Nk | | | | |
| Nes Nes Nes Nds Nds Nds Nds Nds Nds Nds Nds | 537070 | 87 | 547 | 6 |
| Nds Nds Nks Nks Nk | | | | |
| Nes Nes Nes Nds Nds Nds Nds Nds Nds Nds Nds | 537792 | 36 | 548 | 6 |
| Nds Nds Nks Nks Nk | | | | |
| Nes Nes Nes Nds Nds Nds Nds Nds Nds Nds Nds | 537793 | 35 | 549 | 6 |
| Nds Nds Nks Nks Nk | | | | |
| Nes Nes Nes Nds Nds Nds Nds Nds Nds Nds Nds | 537794 | 35 | 550 | 6 |
| Nds Nds Nks Nks Nk | | | | |
| Nes Nes Nes Nds Nds Nds Nds Nds Nds Nds Nds | 537795 | 33 | 551 | 6 |
| Nds Nds Nks Nks Nk | | | | |
| Nes Nes Nes Nds Nds Nds Nds Nds Nds Nds Nds | 537796 | 49 | 552 | 6 |
| Nds Nds Nks Nks Nk | | | | |
| Nes Nes Nes Nds Nds Nds Nds Nds Nds Nds Nds | 537797 | 54 | 553 | 6 |
| Nds Nds Nks Nks Nk | | | | |
| Nes Nes Nes Nds Nds Nds Nds Nds Nds Nds Nds | 537798 | 68 | 554 | 6 |
| Nds Nds Nks Nks Nk | | | | |
| Nes Nes Nes Nds Nds Nds Nds Nds Nds Nds Nds | 537799 | 72 | 555 | 6 |
| Nds Nds Nks Nks Nk | | | | |
| Nes Nes Nes Nds Nds Nds Nds Nds Nds Nds Nds | 537800 | 69 | 556 | 6 |
| Nds Nds Nks Nks Nk | | | | |
| Nes Nes Nes Nds Nds Nds Nds Nds Nds Nds Nds | 537801 | 82 | 557 | 6 |
| Nds Nds Nks Nks Nk | | | | |
| Nes Nes Nes Nds Nds Nds Nds Nds Nds Nds Nds | 537802 | 72 | 558 | 6 |
| Nds Nds Nks Nks Nk | | | | |
| Nes Nes Nes Nds Nds Nds Nds Nds Nds Nds Nds | 537803 | 72 | 559 | 6 |
| Nds Nds Nks Nks Nk | | | | |
| Nes Nes Nes Nds Nds Nds Nds Nds Nds Nds Nds | 537804 | 67 | 560 | 6 |
| Nds Nds Nks Nks Nk | | | | |
| Nes Nes Nes Nds Nds Nds Nds Nds Nds Nds Nds | 537805 | 74 | 561 | 6 |
| Nds Nds Nks Nks Nk | | | | |
| Nes Nes Nes Nds Nds Nds Nds Nds Nds Nds Nds | 537806 | 70 | 562 | 6 |
| Nds Nds Nks Nks Nk | | | | |
| Nes Nes Nes Nds Nds Nds Nds Nds Nds Nds Nds | 537809 | 60 | 563 | 6 |
| Nds Nds Nks Nks Nk | | | | |
| Nes Nes Nes Nds Nds Nds Nds Nds Nds Nds Nds | 537810 | 71 | 564 | 6 |
| Nds Nds Nks Nks Nk | | | | |
| Nes Nes Nes Nds Nds Nds Nds Nds Nds Nds Nds | 537811 | 69 | 565 | 6 |
| Nds Nds Nks Nks Nk | | | | |
| Nes Nes Nes Nds Nds Nds Nds Nds Nds Nds Nds | 537812 | 80 | 566 | 6 |
| Nds Nds Nks Nks Nk | | | | |
| Nes Nes Nes Nds Nds Nds Nds Nds Nds Nds Nds | 537813 | 74 | 567 | 6 |
| Nds Nds Nks Nks Nk | | | | |
| Nes Nes Nes Nds Nds Nds Nds Nds Nds Nds Nds | 537814 | 54 | 568 | 6 |
| Nds Nds Nks Nks Nk | | | | |
| Nes Nes Nes Nds Nds Nds Nds Nds Nds Nds Nds | 537837 | 70 | 569 | 6 |
| Nds Nds Nks Nks Nk | | | | |
| Nes Nes Nes Nds Nds Nds Nds Nds Nds Nds Nds | 537838 | 76 | 570 | 6 |
| Nds Nds Nks Nks Nk | | | | |
| Nes Nes Nes Nds Nds Nds Nds Nds Nds Nds Nds | 537839 | 76 | 571 | 6 |
| Nds Nds Nks Nks Nk | | | | |
| Nes Nes Nes Nds Nds Nds Nds Nds Nds Nds Nds | 537840 | 80 | 572 | 6 |
| Nds Nds Nks Nks Nk | | | | |
| Nes Nes Nes Nds Nds Nds Nds Nds Nds Nds Nds | 537841 | 81 | 573 | 6 |
| Nds Nds Nks Nks Nk | | | | |
| Nes Nes Nes Nds Nds Nds Nds Nds Nds Nds Nds | 537842 | 75 | 574 | 6 |
| Nds Nds Nks Nks Nk | | | | |
| Nes Nes Nes Nds Nds Nds Nds Nds Nds Nds Nds | 537843 | 70 | 575 | 6 |
| Nds Nds Nks Nks Nk | | | | |
| Nes Nes Nes Nds Nds Nds Nds Nds Nds Nds Nds | 537844 | 73 | 576 | 6 |
| Nds Nds Nks Nks Nk | | | | |
| Nes Nes Nes Nds Nds Nds Nds Nds Nds Nds Nds | 537845 | 59 | 577 | 6 |
| Nds Nds Nks Nks Nk | | | | |
| Nes Nes Nes Nds Nds Nds Nds Nds Nds Nds Nds | 537846 | 51 | 578 | 6 |
| Nds Nds Nks Nks Nk | | | | |
| Nes Nes Nes Nds Nds Nds Nds Nds Nds Nds Nds | 537847 | 52 | 579 | 6 |
| Nds Nds Nks Nks Nk | | | | |
| Nes Nes Nes Nds Nds Nds Nds Nds Nds Nds Nds | 537848 | 41 | 580 | 6 |
| Nds Nds Nks Nks Nk | | | | |
| Nes Nes Nes Nds Nds Nds Nds Nds Nds Nds Nds | 537849 | 44 | 581 | 6 |
| Nds Nds Nks Nks Nk | | | | |
| Nes Nes Nes Nds Nds Nds Nds Nds Nds Nds Nds | 538160 | 69 | 582 | 6 |
| Nds Nds Nks Nks Nk | | | | |
| Nes Nes Nes Nds Nds Nds Nds Nds Nds Nds Nds | 538172 | 24 | 583 | 6 |
| Nds Nds Nks Nks Nk | | | | |
| Nes Nes Nes Nds Nds Nds Nds Nds Nds Nds Nds | 538173 | 23 | 584 | 6 |
| Nds Nds Nks Nks Nk | | | | |
| Nes Nes Nes Nds Nds Nds Nds Nds Nds Nds Nds | 538185 | 68 | 585 | 6 |
| Nds Nds Nks Nks Nk | | | | |
| Nes Nes Nes Nds Nds Nds Nds Nds Nds Nds Nds | 538187 | 69 | 585 | 6 |
| Nds Nds Nks Nks Nk | | | | |
| Nes Nes Nes Nds Nds Nds Nds Nds Nds Nds Nds | 538189 | 81 | 587 | 6 |
| Nds Nds Nks Nks Nk | | | | |
| Nes Nes Nes Nds Nds Nds Nds Nds Nds Nds Nds | 538191 | 66 | 588 | 6 |
| Nds Nds Nks Nks Nk | | | | |
| Nes Nes Nes Nds Nds Nds Nds Nds Nds Nds Nds | 538192 | 59 | 589 | 6 |
| Nds Nds Nks Nks Nk | | | | |
| Nes Nes Nes Nds Nds Nds Nds Nds Nds Nds Nds | 538193 | 16 | 590 | 6 |
| Nds Nds Nks Nks Nk | | | | |
| Nes Nes Nes Nds Nds Nds Nds Nds Nds Nds Nds | 538194 | 10 | 591 | 6 |
| Nds Nds Nks Nks Nk | | | | |
| Nes Nes Nes Nds Nds Nds Nds Nds Nds Nds Nds | 538195 | 15 | 592 | 6 |
| Nds Nds Nks Nks Nk | | | | |
| Nes Nes Nes Nds Nds Nds Nds Nds Nds Nds Nds | 538196 | 3 | 593 | 6 |
| Nds Nds Nks Nks Nk | | | | |
| Nes Nes Nes Nds Nds Nds Nds Nds Nds Nds Nds | 538197 | 36 | 594 | 6 |
| Nds Nds Nks Nks Nk | | | | |
| Nes Nes Nes Nds Nds Nds Nds Nds Nds Nds Nds | 538198 | 49 | 595 | 6 |
| Nds Nds Nks Nks Nk | | | | |
| Nes Nes Nes Nds Nds Nds Nds Nds Nds Nds Nds | 538199 | 47 | 596 | 6 |
| Nds Nds Nks Nks Nk | | | | |
| Nes Nes Nes Nds Nds Nds Nds Nds Nds Nds Nds | 538200 | 57 | 597 | 6 |
| Nds Nds Nks Nks Nk | | | | |
| Nes Nes Nes Nds Nds Nds Nds Nds Nds Nds Nds | 538201 | 71 | 598 | 6 |
| Nds Nds Nks Nks Nk | | | | |
| Nes Nes Nes Nds Nds Nds Nds Nds Nds Nds Nds | 538202 | 60 | 599 | 6 |
| Nds Nds Nks Nks Nk | | | | |
| Nes Nes Nes Nds Nds Nds Nds Nds Nds Nds Nds | 538203 | 55 | 600 | 6 |
| Nds Nds Nks Nks Nk | | | | |
| Nes Nes Nes Nds Nds Nds Nds Nds Nds Nds Nds | 538204 | 62 | 601 | 6 |
| Nds Nds Nks Nks Nk | | | | |
| Nes Nes Nes Nds Nds Nds Nds Nds Nds Nds Nds | 538205 | 68 | 602 | 6 |
| Nds Nds Nks Nks Nk | | | | |
| Nes Nes Nes Nds Nds Nds Nds Nds Nds Nds Nds | 538228 | 63 | 603 | 6 |
| Nds Nds Nks Nks Nk | | | | |
| Nes Nes Nes Nds Nds Nds Nds Nds Nds Nds Nds | 538229 | 26 | 604 | 6 |
| Nds Nds Nks Nks Nk | | | | |
| Nes Nes Nes Nds Nds Nds Nds Nds Nds Nds Nds | 538230 | 75 | 605 | 6 |
| Nds Nds Nks Nks Nk | | | | |
| Nes Nes Nes Nds Nds Nds Nds Nds Nds Nds Nds | 538231 | 75 | 606 | 6 |
| Nds Nds Nks Nks Nk | | | | |
| Nes Nes Nes Nds Nds Nds Nds Nds Nds Nds Nds | 538233 | 52 | 607 | 6 |
| Nds Nds Nks Nks Nk | | | | |
| Nes Nes Nes Nds Nds Nds Nds Nds Nds Nds Nds | 538235 | 26 | 608 | 6 |
| Nds Nds Nks Nks Nk | | | | |
| Nes Nes Nes Nds Nds Nds Nds Nds Nds Nds Nds | 538237 | 28 | 609 | 6 |
| Nds Nds Nks Nks Nk | | | | |
| Nes Nes Nes Nds Nds Nds Nds Nds Nds Nds Nds | 538239 | 54 | 610 | 6 |
| Nds Nds Nks Nks Nk | | | | |
| Nes Nes Nes Nds Nds Nds Nds Nds Nds Nds Nds | 538241 | 73 | 611 | 6 |
| Nds Nds Nks Nks Nk | | | | |
| Nes Nes Nes Nds Nds Nds Nds Nds Nds Nds Nds | 538242 | 68 | 612 | 6 |
| Nds Nds Nks Nks Nk | | | | |
| Nes Nes Nes Nds Nds Nds Nds Nds Nds Nds Nds | 538243 | 61 | 613 | 6 |
| Nds Nds Nks Nks Nk | | | | |
| Nes Nes Nes Nds Nds Nds Nds Nds Nds Nds Nds | 538245 | 75 | 614 | 6 |
| Nds Nds Nks Nks Nk | | | | |
| Nes Nes Nes Nds Nds Nds Nds Nds Nds Nds Nds | 538253 | 37 | 615 | 6 |
| Nds Nds Nks Nks Nk | | | | |
| Nes Nes Nes Nds Nds Nds Nds Nds Nds Nds Nds | 538254 | 45 | 616 | 6 |
| Nds Nds Nks Nks Nk | | | | |
| Nes Nes Nes Nds Nds Nds Nds Nds Nds Nds Nds | 538361 | 56 | 617 | 6 |
| Nds Nds Nks Nks Nk | | | | |
| Nes Nes Nes Nds Nds Nds Nds Nds Nds Nds Nds | 538378 | 70 | 618 | 6 |
| Nds Nds Nks Nks Nk | | | | |
| Nes Nes Nes Nds Nds Nds Nds Nds Nds Nds Nds | 538380 | 68 | 619 | 6 |
| Nds Nds Nks Nks Nk | | | | |
| Nes Nes Nes Nds Nds Nds Nds Nds Nds Nds Nds | 538381 | 57 | 620 | 6 |
| Nds Nds Nks Nks Nk | | | | |
| Nes Nes Nes Nds Nds Nds Nds Nds Nds Nds Nds | 540361 | 71 | 621 | 6 |
| Nds Nds Nks Nks Nk | | | | |
| Nes Nes Nes Nds Nds Nds Nds Nds Nds Nds Nds | 540362 | 73 | 622 | 6 |
| Nds Nds Nks Nks Nk | | | | |
| Nes Nes Nes Nds Nds Nds Nds Nds Nds Nds Nds | 540363 | 78 | 623 | 6 |
| Nds Nds Nks Nks Nk | | | | |
| Nes Nes Nes Nds Nds Nds Nds Nds Nds Nds Nds | 540364 | 89 | 624 | 6 |
| Nds Nds Nks Nks Nk | | | | |
| Nes Nes Nes Nds Nds Nds Nds Nds Nds Nds Nds | 540365 | 83 | 625 | 6 |
| Nds Nds Nks Nks Nk | | | | |
| Nes Nes Nes Nds Nds Nds Nds Nds Nds Nds Nds | 540366 | 84 | 626 | 6 |
| Nds Nds Nks Nks Nk | | | | |
| Nes Nes Nes Nds Nds Nds Nds Nds Nds Nds Nds | 540367 | 65 | 627 | 6 |
| Nds Nds Nks Nks Nk | | | | |
| Nes Nes Nes Nds Nds Nds Nds Nds Nds Nds Nds | 540368 | 55 | 628 | 6 |
| Nds Nds Nks Nks Nk | | | | |
| Nes Nes Nes Nds Nds Nds Nds Nds Nds Nds Nds | 540369 | 82 | 629 | 6 |
| Nds Nds Nks Nks Nk | | | | |
| Nes Nes Nes Nds Nds Nds Nds Nds Nds Nds Nds | 540370 | 86 | 630 | 6 |
| Nds Nds Nks Nks Nk | | | | |
| Nes Nes Nes Nds Nds Nds Nds Nds Nds Nds Nds | 540371 | 74 | 631 | 6 |
| Nds Nds Nks Nks Nk | | | | |
| Nes Nes Nes Nds Nds Nds Nds Nds Nds Nds Nds | 540372 | 82 | 632 | 6 |
| Nds Nds Nks Nks Nk | | | | |
| Nes Nes Nes Nds Nds Nds Nds Nds Nds Nds Nds | 540373 | 81 | 633 | 6 |
| Nds Nds Nks Nks Nk | | | | |
| Nes Nes Nes Nds Nds Nds Nds Nds Nds Nds Nds | 540374 | 87 | 634 | 6 |
| Nds Nds Nks Nks Nk | | | | |
| Nes Nes Nes Nds Nds Nds Nds Nds Nds Nds Nds | 540375 | 78 | 635 | 6 |
| Nds Nds Nks Nks Nk | | | | |
| Nes Nes Nes Nds Nds Nds Nds Nds Nds Nds Nds | 540376 | 69 | 636 | 6 |
| Nds Nds Nks Nks Nk | | | | |
| Nes Nes Nes Nds Nds Nds Nds Nds Nds Nds Nds | 540377 | 88 | 637 | 6 |
| Nds Nds Nks Nks Nk | | | | |
| Nes Nes Nes Nds Nds Nds Nds Nds Nds Nds Nds | 540378 | 85 | 638 | 6 |
| Nds Nds Nks Nks Nk | | | | |
| Nes Nes Nes Nds Nds Nds Nds Nds Nds Nds Nds | 540379 | 77 | 639 | 6 |
| Nds Nds Nks Nks Nk | | | | |
| Nes Nes Nes Nds Nds Nds Nds Nds Nds Nds Nds | 540380 | 84 | 640 | 6 |
| Nds Nds Nks Nks Nk | | | | |
| Nes Nes Nes Nds Nds Nds Nds Nds Nds Nds Nds | 540381 | 85 | 641 | 6 |
| Nds Nds Nks Nks Nk | | | | |
| Nes Nes Nes Nds Nds Nds Nds Nds Nds Nds Nds | 540382 | 69 | 642 | 6 |
| Nds Nds Nks Nks Nk | | | | |
| Nes Nes Nes Nds Nds Nds Nds Nds Nds Nds Nds | 540383 | 85 | 643 | 6 |
| Nds Nds Nks Nks Nk | | | | |
| Nes Nes Nes Nds Nds Nds Nds Nds Nds Nds Nds | 540384 | 88 | 644 | 6 |
| Nds Nds Nks Nks Nk | | | | |
| Nes Nes Nes Nds Nds Nds Nds Nds Nds Nds Nds | 540385 | 87 | 645 | 6 |
| Nds Nds Nks Nks Nk | | | | |
| Nes Nes Nes Nds Nds Nds Nds Nds Nds Nds Nds | 540386 | 86 | 646 | 6 |
| Nds Nds Nks Nks Nk | | | | |
| Nes Nes Nes Nds Nds Nds Nds Nds Nds Nds Nds | 540387 | 77 | 647 | 6 |
| Nds Nds Nks Nks Nk | | | | |
| Nes Nes Nes Nds Nds Nds Nds Nds Nds Nds Nds | 540388 | 86 | 648 | 6 |
| Nds Nds Nks Nks Nk | | | | |
| Nes Nes Nes Nds Nds Nds Nds Nds Nds Nds Nds | 540389 | 86 | 649 | 6 |
| Nds Nds Nks Nks Nk | | | | |
| Nes Nes Nes Nds Nds Nds Nds Nds Nds Nds Nds | 540390 | 85 | 650 | 6 |
| Nds Nds Nks Nks Nk | | | | |
| Nes Nes Nes Nds Nds Nds Nds Nds Nds Nds Nds | 540391 | 83 | 651 | 6 |
| Nds Nds Nks Nks Nk | | | | |
| Nes Nes Nes Nds Nds Nds Nds Nds Nds Nds Nds | 540392 | 43 | 652 | 6 |
| Nds Nds Nks Nks Nk | | | | |
| Nes Nes Nes Nds Nds Nds Nds Nds Nds Nds Nds | 540393 | 88 | 653 | 6 |
| Nds Nds Nks Nks Nk | | | | |
| Nes Nes Nes Nds Nds Nds Nds Nds Nds Nds Nds | 540394 | 68 | 654 | 6 |
| Nds Nds Nks Nks Nk | | | | |
| Nes Nes Nes Nds Nds Nds Nds Nds Nds Nds Nds | 540395 | 87 | 655 | 6 |
| Nds Nds Nks Nks Nk | | | | |
| Nes Nes Nes Nds Nds Nds Nds Nds Nds Nds Nds | 540396 | 87 | 656 | 6 |
| Nds Nds Nks Nks Nk | | | | |
| Nes Nes Nes Nds Nds Nds Nds Nds Nds Nds Nds | 540397 | 59 | 657 | 6 |
| Nds Nds Nks Nks Nk | | | | |
| Nes Nes Nes Nds Nds Nds Nds Nds Nds Nds Nds | 540398 | 36 | 658 | 6 |
| Nds Nds Nks Nks Nk | | | | |
| Nes Nes Nes Nds Nds Nds Nds Nds Nds Nds Nds | 540399 | 81 | 659 | 6 |
| Nds Nds Nks Nks Nk | | | | |

### Example 23: High Dose Tolerability of Modified Oligonucleotides Comprising 2′-O-Methoxyethyl (2′-MOE) and 6′-(S)—CH3 Bicyclic Nucleoside (e.g cEt) Modifications Targeting Human Target-X in BALB/c Mice

BALB/c mice were treated at a high dose with ISIS antisense oligonucleotides selected from studies described above and evaluated for changes in the levels of various plasma chemistry markers.

Additionally, the newly designed antisense oligonucleotides were created with the same sequences as the antisense oligonucleotides from the study described above and were also added to this screen targeting intronic repeat regions of Target-X.

The newly designed modified antisense oligonucleotides and their motifs are described in Table 38. The internucleoside linkages throughout each gapmer are phosphorothioate linkages (P═S). Nucleosides followed by “d” indicate 2′-deoxyribonucleosides. Nucleosides followed by “k” indicate 6′-(S)—CH3 bicyclic nucleoside (e.g cEt) nucleosides. Nucleosides followed by “e” indicate 2′-O-methoxyethyl (2′-MOE) nucleosides. “N” indicates modified or naturally occurring nucleobases (A, T, C, G, U, or 5-methyl C).

Each gapmer listed in Table 38 is targeted to the intronic region of human Target-X genomic sequence, designated herein as Target-X. “Start site” indicates the 5′-most nucleoside to which the gapmer is targeted in the human gene sequence. “Stop site” indicates the 3′-most nucleoside to which the gapmer is targeted human gene sequence.

TABLE 38
Modified antisense oligonucleotides targeted to Target-X

| | | | SEQ |
|---|---|---|---|
| | ISIS | SEQ | ID |
| Sequence (5′ to 3′) | No | CODE | NO |
| Nks Nks Nks Nds Nds Nds Nds Nds Nds Nds Nds Nds Nds Nes Nes Ne | 537721 | 509 | 6 |
| Nks Nks Nks Nds Nds Nds Nds Nds Nds Nds Nds Nds Nds Nes Nes Ne | 537738 | 524 | 6 |
| Nks Nks Nks Nds Nds Nds Nds Nds Nds Nds Nds Nds Nds Nes Nes Ne | 537759 | 539 | 6 |
| Nks Nks Nks Nds Nds Nds Nds Nds Nds Nds Nds Nds Nds Nes Nes Ne | 537761 | 541 | 6 |
| Nks Nks Nks Nds Nds Nds Nds Nds Nds Nds Nds Nds Nds Nes Nes Ne | 537763 | 543 | 6 |
| Nks Nks Nks Nds Nds Nds Nds Nds Nds Nds Nds Nds Nds Nes Nes Ne | 537850 | 548 | 6 |
| Nks Nks Nks Nds Nds Nds Nds Nds Nds Nds Nds Nds Nds Nes Nes Ne | 537858 | 556 | 6 |
| Nks Nks Nks Nds Nds Nds Nds Nds Nds Nds Nds Nds Nds Nes Nes Ne | 537864 | 562 | 6 |
| Nks Nks Nks Nds Nds Nds Nds Nds Nds Nds Nds Nds Nds Nes Nes Ne | 537869 | 565 | 6 |
| Nks Nks Nks Nds Nds Nds Nds Nds Nds Nds Nds Nds Nds Nes Nes Ne | 537872 | 568 | 6 |
| Nks Nks Nks Nds Nds Nds Nds Nds Nds Nds Nds Nds Nds Nes Nes Ne | 537897 | 571 | 6 |
| Nks Nks Nks Nds Nds Nds Nds Nds Nds Nds Nds Nds Nds Nes Nes Ne | 540118 | 582 | 6 |
| Nks Nks Nks Nds Nds Nds Nds Nds Nds Nds Nds Nds Nds Nes Nes Ne | 540138 | 602 | 6 |
| Nks Nks Nks Nds Nds Nds Nds Nds Nds Nds Nds Nds Nds Nes Nes Ne | 540139 | 603 | 6 |
| Nks Nks Nks Nds Nds Nds Nds Nds Nds Nds Nds Nds Nds Nes Nes Ne | 540148 | 612 | 6 |
| Nks Nks Nks Nds Nds Nds Nds Nds Nds Nds Nds Nds Nds Nes Nes Ne | 540153 | 617 | 6 |
| Nks Nks Nks Nds Nds Nds Nds Nds Nds Nds Nds Nds Nds Nes Nes Ne | 540155 | 619 | 6 |
| Nes Nes Nks Nds Nds Nds Nds Nds Nds Nds Nds Nds Nds Nks Nks Ne | 540162 | 624 | 6 |
| Nes Nes Nks Nds Nds Nds Nds Nds Nds Nds Nds Nds Nds Nks Nks Ne | 540164 | 626 | 6 |
| Nes Nes Nks Nds Nds Nds Nds Nds Nds Nds Nds Nds Nds Nks Nks Ne | 540168 | 630 | 6 |
| Nes Nes Nks Nds Nds Nds Nds Nds Nds Nds Nds Nds Nds Nks Nks Ne | 540172 | 634 | 6 |
| Nes Nes Nks Nds Nds Nds Nds Nds Nds Nds Nds Nds Nds Nks Nks Ne | 540175 | 637 | 6 |
| Nes Nes Nks Nds Nds Nds Nds Nds Nds Nds Nds Nds Nds Nks Nks Ne | 540176 | 638 | 6 |
| Nes Nes Nks Nds Nds Nds Nds Nds Nds Nds Nds Nds Nds Nks Nks Ne | 540178 | 640 | 6 |
| Nes Nes Nks Nds Nds Nds Nds Nds Nds Nds Nds Nds Nds Nks Nks Ne | 540179 | 641 | 6 |
| Nes Nes Nks Nds Nds Nds Nds Nds Nds Nds Nds Nds Nds Nks Nks Ne | 540181 | 643 | 6 |
| Nes Nes Nks Nds Nds Nds Nds Nds Nds Nds Nds Nds Nds Nks Nks Ne | 540182 | 644 | 6 |
| Nes Nes Nks Nds Nds Nds Nds Nds Nds Nds Nds Nds Nds Nks Nks Ne | 540183 | 645 | 6 |
| Nes Nes Nks Nds Nds Nds Nds Nds Nds Nds Nds Nds Nds Nks Nks Ne | 540184 | 646 | 6 |
| Nes Nes Nks Nds Nds Nds Nds Nds Nds Nds Nds Nds Nds Nks Nks Ne | 540186 | 648 | 6 |
| Nes Nes Nks Nds Nds Nds Nds Nds Nds Nds Nds Nds Nds Nks Nks Ne | 540187 | 649 | 6 |
| Nes Nes Nks Nds Nds Nds Nds Nds Nds Nds Nds Nds Nds Nks Nks Ne | 540188 | 650 | 6 |
| Nes Nes Nks Nds Nds Nds Nds Nds Nds Nds Nds Nds Nds Nks Nks Ne | 540191 | 653 | 6 |
| Nes Nes Nks Nds Nds Nds Nds Nds Nds Nds Nds Nds Nds Nks Nks Ne | 540193 | 655 | 6 |
| Nes Nes Nks Nds Nds Nds Nds Nds Nds Nds Nds Nds Nds Nks Nks Ne | 540194 | 656 | 6 |
| Nes Nes Nks Nds Nds Nds Nds Nds Nds Nds Nds Nds Nds Nks Nks Ne | 544811 | 547 | 6 |
| Nes Nes Nks Nds Nds Nds Nds Nds Nds Nds Nds Nds Nds Nks Nks Ne | 544812 | 545 | 6 |
| Nes Nes Nks Nds Nds Nds Nds Nds Nds Nds Nds Nds Nds Nks Nks Ne | 544813 | 527 | 6 |
| Nes Nes Nks Nds Nds Nds Nds Nds Nds Nds Nds Nds Nds Nks Nks Ne | 544814 | 557 | 6 |
| Nes Nes Nks Nds Nds Nds Nds Nds Nds Nds Nds Nds Nds Nks Nks Ne | 544815 | 546 | 6 |
| Nes Nes Nks Nds Nds Nds Nds Nds Nds Nds Nds Nds Nds Nks Nks Ne | 544816 | 573 | 6 |
| Nes Nes Nks Nds Nds Nds Nds Nds Nds Nds Nds Nds Nds Nks Nks Ne | 544817 | 572 | 6 |
| Nes Nes Nks Nds Nds Nds Nds Nds Nds Nds Nds Nds Nds Nks Nks Ne | 544818 | 566 | 6 |
| Nes Nes Nks Nds Nds Nds Nds Nds Nds Nds Nds Nds Nds Nks Nks Ne | 544819 | 510 | 6 |
| Nes Nes Nks Nds Nds Nds Nds Nds Nds Nds Nds Nds Nds Nks Nks Ne | 544820 | 525 | 6 |
| Nes Nes Nks Nds Nds Nds Nds Nds Nds Nds Nds Nds Nds Nks Nks Ne | 544821 | 567 | 6 |
| Nes Nes Nks Nds Nds Nds Nds Nds Nds Nds Nds Nds Nds Nks Nks Ne | 544826 | 537 | 6 |
| Nes Nes Nks Nds Nds Nds Nds Nds Nds Nds Nds Nds Nds Nks Nks Ne | 544827 | 538 | 6 |
| Nes Nes Nks Nds Nds Nds Nds Nds Nds Nds Nds Nds Nds Nks Nks Ne | 544828 | 539 | 6 |
| Nes Nes Nks Nds Nds Nds Nds Nds Nds Nds Nds Nds Nds Nks Nks Ne | 544829 | 540 | 6 |
| Nes Nes Nks Nds Nds Nds Nds Nds Nds Nds Nds Nds Nds Nks Nks Ne | 544830 | 541 | 6 |
| Nes Nes Nks Nds Nds Nds Nds Nds Nds Nds Nds Nds Nds Nks Nks Ne | 545471 | 542 | 6 |
| Nes Nes Nks Nds Nds Nds Nds Nds Nds Nds Nds Nds Nds Nks Nks Ne | 545472 | 543 | 6 |
| Nes Nes Nks Nds Nds Nds Nds Nds Nds Nds Nds Nds Nds Nks Nks Ne | 545473 | 544 | 6 |
| Nes Nes Nks Nds Nds Nds Nds Nds Nds Nds Nds Nds Nds Nks Nks Ne | 545474 | 558 | 6 |
| Nes Nes Nks Nds Nds Nds Nds Nds Nds Nds Nds Nds Nds Nks Nks Ne | 545475 | 559 | 6 |
| Nes Nes Nks Nds Nds Nds Nds Nds Nds Nds Nds Nds Nds Nks Nks Ne | 545476 | 560 | 6 |
| Nes Nes Nks Nds Nds Nds Nds Nds Nds Nds Nds Nds Nds Nks Nks Ne | 545477 | 561 | 6 |
| Nes Nes Nks Nds Nds Nds Nds Nds Nds Nds Nds Nds Nds Nks Nks Ne | 545478 | 562 | 6 |
| Nes Nes Nks Nds Nds Nds Nds Nds Nds Nds Nds Nds Nds Nks Nks Ne | 545479 | 556 | 6 |
| Nks Nks Nks Nds Nds Nds Nds Nds Nds Nds Nds Nds Nds Nes Nes Ne | 537727 | 514 | 6 |

### Treatment

Male BALB/c mice were injected subcutaneously with a single dose of 200 mg/kg of ISIS 422142, ISIS 457851, ISIS 473294, ISIS 473295, ISIS 473327, ISIS 484714, ISIS 515334, ISIS 515338, ISIS 515354, ISIS 515366, ISIS 515380, ISIS 515381, ISIS 515382, ISIS 515384, ISIS 515386, ISIS 515387, ISIS 515388, ISIS 515406, ISIS 515407, ISIS 515408, ISIS 515422, ISIS 515423, ISIS 515424, ISIS 515532, ISIS 515533, ISIS 515534, ISIS 515538, ISIS 515539, ISIS 515558, ISIS 515656, ISIS 515575, ISIS 515926, ISIS 515944, ISIS 515945, ISIS 515948, ISIS 515949, ISIS 515951, ISIS 515952, ISSI 516003, ISIS 516055, ISIS 516057, ISIS 516060, ISIS 516062, ISIS 529126, ISIS 529146, ISIS 529166, ISIS 529170, ISIS 529172, ISIS 529173, ISIS 529174, ISIS 529175, ISSI 529176, ISIS 529182, ISIS 529183, ISIS 529186, ISIS 529282, ISIS 529304, ISIS 529306, ISIS 529360, ISIS 529450, ISIS 529459, ISIS 529460, ISIS 529461, ISIS 529547, ISIS 529550, ISIS 529551, ISIS 529553, ISIS 529557, ISIS 529562, ISIS 529563, ISIS 529564, ISIS 529565, ISIS 529575, ISIS 529582, ISIS 529589, ISIS 529607, ISIS 529614, ISIS 529632, ISIS 529650, ISIS 529651, ISIS 529657, ISIS 529663, ISIS 529725, ISIS 529745, ISIS 529765, ISIS 529785, ISIS 529804, ISIS 529818, ISIS 529823, ISIS 529854, ISIS 534528, ISIS 534534, ISIS 534594, ISIS 534660, ISIS 534663, ISIS 534664, ISIS 534676, ISIS 534677, ISIS 537679, ISIS 537683, ISIS 534693, ISIS 534701, ISIS 534716, ISIS 534730, ISIS 534765, ISIS 534795, ISIS 534796, ISIS 534797, ISIS 534798, ISIS 534799, ISIS 534800, ISIS 534802, ISIS 534806, ISSI 534830, ISIS 534838, ISIS 534888, ISIS 534890, ISIS 534898, ISIS 534911, ISIS 534920, ISIS 534926, ISIS 534937, ISIS 534950, ISSI 534956, ISIS 534980, ISIS 534986, ISIS 535010, ISIS 535043, ISIS 535049, ISIS 535076, ISIS 535082, ISSI 535142, ISIS 537024, ISIS 537030, ISIS 537041, ISIS 537062, ISIS 537064, ISIS 537066, ISIS 537721, ISIS 537727, ISIS 537738, ISIS 537759, ISIS 537761, ISIS 537763, ISIS 537792, ISIS 537800, ISIS 537806, ISIS 537811, ISIS 537814, ISIS 537839, ISIS 537850, ISSI 537858, ISIS 537864, ISIS 537869, ISIS 537872, ISIS 537897, ISIS 538160, ISIS 538196, ISIS 538205, ISIS 538228, ISIS 538242, ISIS 538361, ISIS 538380, ISIS 540118, ISIS 540138, ISIS 540139, ISIS 540148, ISIS 540153, ISIS 540155, ISIS 540162, ISIS 540164, ISIS 540168, ISIS 540172, ISIS 540175, ISIS 540176, ISIS 540178, ISIS 540179, ISIS 540181, ISIS 540182, ISIS 540183, ISIS 540184, ISIS 540186, ISIS 540187, ISIS 540188, ISIS 540191, ISIS 540193, ISIS 540194, ISIS 544811, ISIS 544812, ISIS 544813, ISIS 544814, ISIS 544815, ISIS 544816, ISIS 544817, ISIS 544818, ISIS 544819, ISIS 544820, ISIS 544821, ISIS 544826, ISIS 544827, ISIS 544828, ISIS 544829, ISIS 544830, ISIS 545471, ISIS 545472, ISIS 545473, ISIS 545474, ISIS 545475, ISIS 545476, ISIS 545477, ISIS 545478, and ISIS 545479. One set of male BALB/c mice was injected with a single dose of PBS. Mice were euthanized 96 hours later, and organs and plasma were harvested for further analysis.

### Plasma Chemistry Markers

To evaluate the effect of ISIS oligonucleotides on liver and kidney function, plasma levels of transaminases, bilirubin, albumin, and BUN were measured using an automated clinical chemistry analyzer (Hitachi Olympus AU400e, Melville, NY).

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 529166, ISIS 529170, ISIS 529175, ISIS 529176, ISIS 529186, ISIS 529282, ISIS 529360, ISIS 529450, ISIS 529459, ISIS 529460, ISIS 529547, ISIS 529549, ISIS 529551, ISIS 529553, ISIS 529557, ISIS 529562, ISIS 529575, ISIS 529582, ISIS 529607, ISIS 529589, ISIS 529632, ISIS 529657, ISIS 529725, ISIS 529745, ISIS 529785, ISIS 529799, ISIS 529804, ISIS 529818, ISIS 529823, ISIS 534950, ISIS 534980, ISIS 535010, ISIS 537030, ISIS 537041, ISIS 537062, ISIS 537064, ISIS 537066, ISIS 537759, ISIS 537792, ISIS 537800, ISIS 537839, ISIS 538228, ISIS 473294, ISIS 473295, ISIS 484714, ISIS 515338, ISIS 515366, ISIS 515380, ISIS 515381, ISIS 515387, ISIS 515408, ISIS 515423, ISIS 515424, ISIS 515532, ISIS 515534, ISIS 515538, ISIS 515539, ISIS 515558, ISIS 515575, ISIS 515926, ISIS 515944, ISIS 515945, ISIS 515951, ISIS 515952, ISIS 529126, ISIS 529765, ISIS 534528, ISIS 534534, ISIS 534594, ISIS 534663, ISIS 534676, ISIS 534677, ISIS 534679, ISIS 534683, ISIS 534693, ISIS 534701, ISIS 534716, ISIS 534730, ISIS 534806, ISIS 534830, ISIS 534838, ISIS 534890, ISIS 534898, ISIS 534911, ISIS 534937, ISIS 534956, ISIS 534986, ISIS 535043, ISIS 535049, ISIS 535076, ISIS 535082, ISIS 535142, ISIS 538160, ISIS 538242, ISIS 538361, ISIS 538380, ISIS 534795, ISIS 534796, ISIS 534797, ISIS 540162, ISIS 540164, ISIS 540168, ISIS 540172, ISIS 540175, ISIS 540176, ISIS 540178, ISIS 540179, ISIS 540181, ISIS 540182, ISIS 540183, ISIS 540184, ISIS 540186, ISIS 540187, ISIS 540188, ISIS 540191, ISIS 540193, ISIS 540194, ISIS 544813, ISIS 544814, ISIS 544816, ISIS 544826, ISIS 544827, ISIS 544828, ISIS 544829, ISIS 545473, and ISIS 545474 were considered very tolerable in terms of liver function. Based on these criteria, ISIS 529173, ISIS 529854, ISIS 529614, ISIS 515386, ISIS 515388, ISIS 515949, ISIS 544817, and ISIS 545479 were considered tolerable in terms of liver function.

### Example 24: Tolerability of Antisense Oligonucleotides Targeting Human Target-X in Sprague-Dawley Rats

Sprague-Dawley rats are a multipurpose model used for safety and efficacy evaluations. The rats were treated with ISIS antisense oligonucleotides from the studies described in the Examples above and evaluated for changes in the levels of various plasma chemistry markers.

### Treatment

Six-eight week old male Sprague-Dawley rats were maintained on a 12-hour light/dark cycle and fed ad libitum with Teklad normal rat chow. Groups of four Sprague-Dawley rats each were injected subcutaneously twice a week for 6 weeks with 25 mg/kg of ISIS 473286, ISIS 473547, ISIS 473567, ISIS 473589, ISIS 473630, ISIS 484559, ISIS 515636, ISIS 515640, ISIS 515641, ISIS 515655, ISIS 515657, ISIS 516046, ISIS 516048, ISIS 516051, ISIS 516052, and ISIS 516062. A group of four Sprague-Dawley rats was injected subcutaneously twice a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured using an automated clinical chemistry analyzer (Hitachi Olympus AU400e, Melville, NY). Plasma levels of ALT (alanine transaminase) and AST (aspartate transaminase) were measured. Plasma levels of Bilirubin and BUN were also measured using the same clinical chemistry analyzer.

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 473286, ISIS 473547, ISSI 473589, ISIS 473630, ISIS 484559, ISIS 515636, ISIS 515640, ISIS 515655, ISIS 516046, and ISIS 516051 were considered very tolerable in terms of liver function. Based on these criteria, ISIS 473567, ISIS 515641, ISIS 515657, ISIS 516048, and ISIS 516051 were considered tolerable in terms of liver function.

### Example 25: Tolerability of Chimeric Antisense Oligonucleotides Comprising 2′-O-Methoxyethyl (2′-MOE) Modifications Targeting Human Target-X in Sprague-Dawley Rats

Sprague-Dawley rats were treated with ISIS antisense oligonucleotides from the studies described in the Examples above and evaluated for changes in the levels of various plasma chemistry markers.

### Treatment

Six-eight week old male Sprague-Dawley rats were maintained on a 12-hour light/dark cycle and fed ad libitum with Purina normal rat chow. Groups of four Sprague-Dawley rats each were injected subcutaneously twice a week for 6 weeks with 50 mg/kg of ISIS 407936, ISIS 416507, ISIS 416508, ISIS 490208, ISIS 490279, ISIS 490323, ISIS 490368, ISIS 490396, ISIS 490803, ISIS 491122, ISIS 513419, ISIS 513446, ISIS 513454, ISIS 513455, ISIS 513456, ISIS 513504, ISIS 513507, and ISIS 513508. A group of four Sprague-Dawley rats was injected subcutaneously twice a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured using an automated clinical chemistry analyzer (Hitachi Olympus AU400e, Melville, NY). Plasma levels of Bilirubin and BUN were also measured using the same clinical chemistry analyzer.

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 416507, ISIS 490208, ISIS 490368, ISIS 490396, ISIS 490803, ISIS 491122, ISIS 513446, ISIS 513454, ISIS 513455, ISIS 513456, ISIS 513504, and ISIS 513508 were considered very tolerable in terms of liver function. Based on these criteria, ISIS 407936, ISIS 416508, ISIS 490279, and ISIS 513507 were considered tolerable in terms of liver function.

### Example 26: Tolerability of Chimeric Antisense Oligonucleotides Comprising 2′-O-Methoxyethyl (2′-MOE) Modifications Targeting Human Target-X in CD-1 Mice

CD-1 mice are a multipurpose mice model, frequently utilized for safety and efficacy testing. The mice were treated with ISIS antisense oligonucleotides selected from studies described above and evaluated for changes in the levels of various plasma chemistry markers.

### Treatment

Groups of 3 male CD-1 mice each were injected subcutaneously twice a week for 6 weeks with 50 mg/kg of ISIS 473244, ISIS 473295, ISIS 484714, ISIS 515386, ISIS 515424, ISIS 515534, ISIS 515558, ISIS 515926, ISIS 515949, ISIS 515951, ISIS 515952, ISIS 529126, ISIS 529166, ISIS 529173, ISIS 529186, ISIS 529360, ISIS 529461, ISIS 529553, ISIS 529564, ISIS 529582, ISIS 529614, ISIS 529725, ISIS 529745, ISIS 529765, ISIS 529785, ISIS 529799, ISIS 529818, ISIS 529823, ISIS 534528, ISIS 534594, and ISIS 534664. One group of male CD-1 mice was injected subcutaneously twice a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, bilirubin, albumin, and BUN were measured using an automated clinical chemistry analyzer (Hitachi Olympus AU400e, Melville, NY).

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 473295, ISIS 473714, ISIS 515558, ISIS 515926, 515951, ISIS 515952, ISIS 529126, ISIS 529166, 529564, ISIS 529582, ISIS 529614, ISIS 529725, ISIS 529765, ISIS 529799, ISIS 529823, and ISIS 534594 were considered very tolerable in terms of liver function. Based on these criteria, ISIS 515424, ISIS 515534, ISIS 515926, ISIS 529785, and ISIS 534664 were considered tolerable in terms of liver function.

### Example 27: Tolerability of Chimeric Antisense Oligonucleotides Comprising 2′-O-Methoxyethyl (2′-MOE) Modifications Targeting Human Target-X in CD-1 Mice

CD-1 mice were treated with ISIS antisense oligonucleotides selected from studies described above and evaluated for changes in the levels of various plasma chemistry markers.

### Treatment

Groups of 3 male CD-1 mice each were injected subcutaneously twice a week for 6 weeks with 100 mg/kg of ISIS 490208, ISIS 490279, ISIS 490323, ISIS 490368, ISIS 490396, ISIS 490803, ISIS 491122, ISIS 513419, ISIS 513446, ISIS 513454, ISIS 513455, ISIS 513456, ISIS 513504, ISIS 513507, and ISIS 513508. Groups of 3 male CD-1 mice each were injected subcutaneously twice a week for 6 weeks with 100 mg/kg of ISIS 407936, ISIS 416507, and ISIS 416508, which are gapmers described in a previous publication. One group of male CD-1 mice was injected subcutaneously twice a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, bilirubin, and BUN were measured using an automated clinical chemistry analyzer (Hitachi Olympus AU400e, Melville, NY).

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 407936, ISIS 416507, ISIS 490279, ISIS 490368, ISIS 490396, ISIS 490803, ISIS 491122, ISIS 513446, ISIS 513454, ISIS 513456, and ISIS 513504 were considered very tolerable in terms of liver function. Based on these criteria, ISIS 490208, ISIS 513455, ISIS 513507, and ISIS 513508 were considered tolerable in terms of liver function.

### Example 28: Efficacy of Modified Oligonucleotides Comprising 2′-O-Methoxyethyl (2′-MOE) and 6′-(S)—CH₃ Bicyclic Nucleoside (e.g cEt) Modifications Targeting Human Target-X in Transgenic Mice

Transgenic mice were treated with ISIS antisense oligonucleotides selected from studies described above and evaluated for efficacy.

### Treatment

Groups of 2-3 male and female transgenic mice were injected subcutaneously twice a week for 3 weeks with 5 mg/kg/week of ISIS 473244, ISIS 473295, ISIS 484714, ISIS 515926, ISIS 515951, ISIS 515952, ISIS 516062, ISIS 529126, ISIS 529553, ISIS 529745, ISIS 529799, ISIS 534664, ISIS 534826, ISIS 540168, ISIS 540175, ISIS 544826, ISIS 544827, ISIS 544828, and ISIS 544829. One group of mice was injected subcutaneously twice a week for 3 weeks with PBS. Mice were euthanized 48 hours after the last dose, and organs and plasma were harvested for further analysis.

### Protein Analysis

Plasma protein levels of Target-X were estimated using a Target-X ELISA kit (purchased from Hyphen Bio-Med). Results are presented as percent inhibition of Target-X, relative to control. As shown in Table 39, several antisense oligonucleotides achieved reduction of human Target-X over the PBS control. ‘n.d.’ indicates that the value for that particular oligonucleotide was not measured.

TABLE 39
Percent inhibition of Target-X plasma
protein levels in transgenic mice

| ISIS No | % inhibition |
|---|---|
| 473244 | 2 |
| 473295 | 13 |
| 484714 | 19 |
| 515926 | 11 |
| 515951 | 13 |
| 515952 | 0 |
| 516062 | 62 |
| 529126 | 0 |
| 529553 | 0 |
| 529745 | 22 |
| 529799 | 26 |
| 534664 | 32 |
| 534826 | n.d. |
| 540168 | 94 |
| 540175 | 98 |
| 544813 | 0 |
| 544826 | 23 |
| 544827 | 60 |
| 544828 | 33 |
| 544829 | 53 |

### Example 29: Efficacy of Modified Oligonucleotides Comprising 2′-Methoxyethyl (2′-MOE) and 6′-(S)—CH₃ Bicyclic Nucleoside (e.g cEt) Modifications Targeting Human Target-X in Transgenic Mice

Transgenic mice were treated with ISIS antisense oligonucleotides selected from studies described above and evaluated for efficacy.

### Treatment

Groups of 2-3 male and female transgenic mice were injected subcutaneously twice a week for 3 weeks with 1 mg/kg/week of ISIS 407936, ISIS 490197, ISIS 490275, ISIS 490278, ISIS 490279, ISIS 490323, ISIS 490368, ISIS 490396, ISIS 490803, ISIS 491122, ISIS 513446, ISIS 513447, ISIS 513504, ISIS 516062, ISIS 529166, ISIS 529173, ISIS 529360, ISIS 529725, ISIS 534557, ISIS 534594, ISIS 534664, ISIS 534688, ISIS 534689, ISIS 534915, ISIS 534916, ISIS 534917, and ISIS 534980. One group of mice was injected subcutaneously twice a week for 3 weeks with PBS. Mice were euthanized 48 hours after the last dose, and organs and plasma were harvested for further analysis.

### Protein Analysis

Plasma protein levels of Target-X were estimated using a Target-X ELISA kit (purchased from Hyphen Bio-Med). Results are presented as percent inhibition of Target-X, relative to control. As shown in Table 40, several antisense oligonucleotides achieved reduction of human Target-X over the PBS control.

TABLE 40
Percent inhibition of Target-X plasm
protein levels in transgenic mice

| ISIS No | % inhibition |
|---|---|
| 407936 | 28 |
| 490197 | 50 |
| 490275 | 21 |
| 490278 | 20 |
| 490279 | 59 |
| 490323 | 54 |
| 490368 | 22 |
| 490396 | 31 |
| 490803 | 30 |
| 491122 | 51 |
| 513446 | 29 |
| 513447 | 44 |
| 513504 | 45 |
| 516062 | 75 |
| 529166 | 37 |
| 529173 | 64 |
| 529360 | 43 |
| 529725 | 53 |
| 534557 | 76 |
| 534594 | 40 |
| 534664 | 14 |
| 534687 | 12 |
| 534688 | 48 |
| 534689 | 25 |
| 534915 | 40 |
| 534916 | 45 |
| 534917 | 66 |
| 534980 | 62 |

### Example 30: Tolerability of Antisense Oligonucleotides Targeting Human Target-X in Sprague-Dawley Rats

Sprague-Dawley rats were treated with ISIS antisense oligonucleotides from the studies described in the Examples above and evaluated for changes in the levels of various plasma chemistry markers.

### Treatment

Six-eight week old male Sprague-Dawley rats were maintained on a 12-hour light/dark cycle and fed ad libitum with Teklad normal rat chow. Groups of four Sprague-Dawley rats each were injected subcutaneously twice a week for 4 weeks with ISIS 515380, ISIS 515381, ISIS 515387, ISIS 529175, ISIS 529176, ISIS 529575, ISIS 529804, and ISIS 537064. Doses 1, 5, 6, 7, and 8 were 25 mg/kg; dose 2 was 75 mg/kg; doses 3 and 4 were 50 mg/kg. One group of four Sprague-Dawley rats was injected subcutaneously twice a week for 4 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured using an automated clinical chemistry analyzer (Hitachi Olympus AU400e, Melville, NY). Plasma levels of ALT (alanine transaminase) and AST (aspartate transaminase) were measured. Plasma levels of Bilirubin and BUN were also measured using the same clinical chemistry analyzer.

ISIS oligonucleotides that did not cause any increase in the levels of transaminases, or which caused increase in the levels within three times the upper limit of normal levels of transaminases were deemed very tolerable. ISIS oligonucleotides that caused increase in the levels of transaminases between three times and seven times the upper limit of normal levels were deemed tolerable. Based on these criteria, ISIS 515380, ISIS 515387, ISIS 529175, ISIS 529176, ISIS 529804, and ISIS 537064 were considered very tolerable in terms of liver function. Based on these criteria, ISIS 515381 was considered tolerable in terms of liver function.

### Example 31: Efficacy of Antisense Oligonucleotides Targeting Human Target-X in Transgenic Mice

Transgenic mice were treated with ISIS antisense oligonucleotides selected from studies described above and evaluated for efficacy.

### Treatment

Two groups of 3 male and female transgenic mice were injected subcutaneously twice a week for 2 weeks with 0.5 mg/kg/week or 1.5 mg/kg/week of ISIS 407935 and ISIS 513455. Another group of mice was subcutaneously twice a week for 2 weeks with 0.6 mg/kg/week or 2.0 mg/kg/week of ISIS 473286. Another 16 groups of mice were subcutaneously twice a week for 2 weeks with 0.1 mg/kg/week or 0.3 mg/kg/week of ISIS 473589, ISIS 515380, ISIS 515423, ISIS 529804, ISIS 534676, ISIS 534796, ISIS 540162, ISIS 540164, ISIS 540175, ISIS 540179, ISIS 540181, ISIS 540182, ISIS 540186, ISIS 540191, ISIS 540193, ISIS 544827, or ISIS 545474. Another 3 groups of mice were injected subcutaneously twice a week for 2 weeks with 0.3 mg/kg/week of ISIS 516062, ISIS 534528 or ISIS 534693. One group of mice was injected subcutaneously twice a week for 2 weeks with PBS. Mice were euthanized 48 hours after the last dose, and organs and plasma were harvested for further analysis.

### Protein Analysis

Plasma protein levels of Target-X were estimated using a Target-X ELISA kit (purchased from Hyphen Bio-Med). Results are presented as percent inhibition of Target-X, relative to control. As shown in Table 41, several antisense oligonucleotides achieved reduction of human Target-X over the PBS control.

TABLE 41
Percent inhibition of Target-X plasma
protein levels in transgenic mice

| | Dose | % |
|---|---|---|
| ISIS No | (mg/kg/wk) | inhibition |
| 407935 | 1.5 | 65 |
| | 0.5 | 31 |
| 513455 | 1.5 | 64 |
| | 0.5 | 52 |
| 473286 | 2 | 67 |
| | 0.6 | 11 |
| 473589 | 0.3 | 42 |
| | 0.1 | 12 |
| 515380 | 0.3 | 64 |
| | 0.1 | 32 |
| 515423 | 0.3 | 72 |
| | 0.1 | 37 |
| 529804 | 0.3 | 36 |
| | 0.1 | 24 |
| 534676 | 0.3 | 31 |
| | 0.1 | 18 |
| 534796 | 0.3 | 54 |
| | 0.1 | 43 |
| 540162 | 0.3 | 84 |
| | 0.1 | 42 |
| 540164 | 0.3 | 25 |
| | 0.1 | 17 |
| 540175 | 0.3 | 90 |
| | 0.1 | 55 |
| 540179 | 0.3 | 29 |
| | 0.1 | 24 |
| 540181 | 0.3 | 53 |
| | 0.1 | 0 |
| 540182 | 0.3 | 78 |
| | 0.1 | 21 |
| 540186 | 0.3 | 72 |
| | 0.1 | 46 |
| 540191 | 0.3 | 62 |
| | 0.1 | 35 |
| 540193 | 0.3 | 74 |
| | 0.1 | 46 |
| 544827 | 0.3 | 28 |
| | 0.1 | 19 |
| 545474 | 0.3 | 59 |
| | 0.1 | 0 |
| 516062 | 0.3 | 33 |
| 534528 | 0.3 | 41 |
| 534693 | 0.3 | 34 |

### Example 32: Tolerability of Antisense Oligonucleotides Targeting Human Target-X in Sprague-Dawley Rats

Sprague-Dawley rats were treated with ISIS antisense oligonucleotides from the studies described in the Examples above and evaluated for changes in the levels of various plasma chemistry markers.

### Treatment

Five-six week old male Sprague-Dawley rats were maintained on a 12-hour light/dark cycle and fed ad libitum with Teklad normal rat chow. Groups of four Sprague-Dawley rats each were injected subcutaneously twice a week for 4 weeks with 50 mg/kg of ISIS 515423, ISIS 515424, ISIS 515640, ISIS 534676, ISIS 534796, ISIS 534797, ISIS 540162, ISIS 540164, ISIS 540172, ISIS 540175, ISIS 540179, ISIS 540181, ISIS 540182, ISIS 540183, ISIS 540186, ISIS 540191, and ISIS 545474. A group of four Sprague-Dawley rats was injected subcutaneously twice a week for 4 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured using an automated clinical chemistry analyzer (Hitachi Olympus AU400e, Melville, NY). Plasma levels of ALT (alanine transaminase) and AST (aspartate transaminase) were measured. Plasma levels of Bilirubin and BUN were also measured using the same clinical chemistry analyzer.

ISIS oligonucleotides that did not cause any increase in the levels of transaminases, or which caused an increase within three times the upper limit of normal (ULN) were deemed very tolerable. ISIS oligonucleotides that caused an increase in the levels of transaminases between three times and seven times the ULN were deemed tolerable. Based on these criteria, ISIS 540164, ISIS 540172, and ISIS 540175 were considered very tolerable in terms of liver function. Based on these criteria, ISIS 534676, ISIS 534796, ISIS 534797, ISIS 540162, and ISIS 540179 were considered tolerable in terms of liver function.

### Example 33: Dose-Dependent Antisense Inhibition of Human Target-X in Hep3B Cells

Antisense oligonucleotides selected from the studies described above were tested at various doses in Hep3B cells. Cells were plated at a density of 20,000 cells per well and transfected using electroporation with 0.05 μM, 0.15 μM, 0.44 μM, 1.33 μM, and 4.00 μM concentrations of antisense oligonucleotide, as specified in Table 42. After a treatment period of approximately 16 hours, RNA was isolated from the cells and Target-X mRNA levels were measured by quantitative real-time PCR. Human Target-X primer probe set RTS2927 was used to measure mRNA levels. Target-X mRNA levels were adjusted according to total RNA content, as measured by RIBOGREEN®. Results are presented as percent inhibition of Target-X, relative to untreated control cells.

The half maximal inhibitory concentration (IC₅₀) of each oligonucleotide is also presented in Table 42. As illustrated in Table 42, Target-X mRNA levels were reduced in a dose-dependent manner in several of the antisense oligonucleotide treated cells.

TABLE 42
Dose-dependent antisense inhibition of human
Target-X in Hep3B cells using electroporation

| | 0.05 | 0.15 | 0.44 | 1.33 | 4.00 | IC₅₀ |
|---|---|---|---|---|---|---|
| ISIS No | μM | μM | μM | μM | μM | (μM) |
| 473286 | 0 | 1 | 13 | 12 | 15 | >4.0 |
| 457851 | 23 | 32 | 57 | 80 | 93 | 0.3 |
| 473286 | 3 | 20 | 43 | 71 | 88 | 0.5 |
| 473286 | 15 | 26 | 24 | 28 | 36 | >4.0 |
| 473286 | 6 | 3 | 10 | 26 | 29 | >4.0 |
| 473327 | 14 | 28 | 35 | 67 | 90 | 0.5 |
| 473589 | 29 | 53 | 76 | 89 | 95 | 0.1 |
| 515380 | 44 | 72 | 85 | 93 | 95 | <0.05 |
| 515423 | 43 | 64 | 87 | 95 | 98 | <0.05 |
| 515424 | 38 | 55 | 85 | 92 | 97 | 0.1 |
| 515636 | 21 | 33 | 74 | 82 | 93 | 0.2 |
| 516046 | 29 | 23 | 29 | 48 | 78 | 0.9 |
| 516048 | 35 | 24 | 41 | 67 | 87 | 0.4 |
| 516052 | 18 | 6 | 48 | 63 | 80 | 0.6 |
| 516062 | 24 | 14 | 21 | 47 | 68 | 1.6 |
| 529166 | 16 | 47 | 75 | 87 | 94 | 0.2 |
| 529173 | 14 | 49 | 77 | 91 | 96 | 0.2 |
| 529175 | 30 | 69 | 88 | 93 | 96 | 0.1 |
| 529176 | 34 | 63 | 85 | 93 | 96 | 0.1 |
| 529360 | 35 | 53 | 74 | 91 | 93 | 0.1 |
| 529725 | 53 | 69 | 85 | 92 | 95 | <0.05 |
| 529804 | 37 | 41 | 71 | 90 | 94 | 0.1 |
| 534528 | 50 | 68 | 78 | 93 | 97 | <0.05 |
| 534557 | 48 | 78 | 90 | 94 | 95 | <0.05 |
| 534594 | 39 | 47 | 76 | 87 | 94 | 0.1 |
| 534676 | 29 | 20 | 40 | 64 | 87 | 0.5 |
| 534687 | 41 | 37 | 56 | 80 | 93 | 0.2 |
| 534688 | 16 | 56 | 88 | 94 | 96 | 0.1 |
| 534689 | 21 | 59 | 82 | 94 | 95 | 0.1 |
| 534693 | 18 | 58 | 81 | 93 | 95 | 0.1 |
| 534795 | 19 | 43 | 68 | 90 | 94 | 0.2 |
| 534796 | 25 | 59 | 80 | 93 | 96 | 0.1 |
| 534890 | 31 | 55 | 77 | 90 | 96 | 0.1 |
| 534898 | 22 | 61 | 80 | 94 | 97 | 0.1 |
| 534915 | 19 | 26 | 51 | 77 | 94 | 0.3 |
| 534916 | 20 | 36 | 66 | 86 | 93 | 0.2 |
| 534917 | 34 | 53 | 82 | 89 | 94 | 0.1 |
| 540162 | 40 | 64 | 84 | 90 | 92 | <0.05 |
| 540164 | 34 | 60 | 83 | 91 | 92 | 0.1 |
| 540168 | 51 | 79 | 90 | 92 | 94 | <0.05 |
| 540172 | 40 | 66 | 80 | 88 | 92 | <0.05 |
| 540175 | 30 | 61 | 80 | 88 | 91 | 0.1 |
| 540176 | 7 | 17 | 50 | 75 | 85 | 0.5 |
| 540179 | 11 | 22 | 25 | 16 | 19 | >4.0 |
| 540181 | 19 | 46 | 72 | 86 | 91 | 0.2 |
| 540182 | 16 | 66 | 83 | 86 | 92 | 0.1 |
| 540183 | 39 | 74 | 87 | 92 | 93 | <0.05 |
| 540186 | 31 | 69 | 85 | 91 | 94 | 0.1 |
| 540191 | 38 | 54 | 80 | 88 | 91 | 0.1 |
| 540193 | 57 | 67 | 84 | 94 | 97 | <0.05 |
| 540194 | 30 | 45 | 62 | 77 | 91 | 0.2 |
| 544827 | 37 | 42 | 67 | 82 | 96 | 0.1 |
| 544829 | 26 | 41 | 42 | 71 | 93 | 0.3 |
| 545473 | 28 | 27 | 49 | 80 | 97 | 0.3 |
| 545474 | 23 | 27 | 55 | 84 | 96 | 0.3 |

### Example 34: Tolerability of Antisense Oligonucleotides Targeting Human Target-X in CD-1 Mice

CD-1 mice were treated with ISIS antisense oligonucleotides selected from studies described above and evaluated for changes in the levels of various plasma chemistry markers.

### Treatment

Two groups of 4 male 6-8 week old CD-1 mice each were injected subcutaneously twice a week for 6 weeks with 50 mg/kg of ISIS 407935 and ISIS 490279. Another seven groups of 4 male 6-8 week old CD-1 mice each were injected subcutaneously twice a week for 6 weeks with 25 mg/kg of ISIS 473589, ISIS 529804, ISIS 534796, ISIS 540162, ISIS 540175, ISIS 540182, and ISIS 540191. One group of male CD-1 mice was injected subcutaneously twice a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, bilirubin, albumin, and BUN were measured using an automated clinical chemistry analyzer (Hitachi Olympus AU400e, Melville, NY). The results are presented in Table 43. Treatment with the newly designed antisense oligonucleotides were more tolerable compared to treatment with ISIS 407935 (disclosed in an earlier publication), which caused elevation of ALT levels greater than seven times the upper limit of normal (ULN).

TABLE 43
Effect of antisense oligonucleotide treatment on liver function in CD-1 mice

| | | Dose | | AST | BUN | Bilirubin |
|---|---|---|---|---|---|---|
| | Motif | (mg/kg/wk) | ALT(IU/L) | (IU/L) | (mg/dL) | (mg/dL) |
| PBS | — | — | 37 | 47 | 28 | 0.2 |
| 407935 | e5-d(10)-e5 | 100 | 373 | 217 | 24 | 0.2 |
| 490279 | kdkdk-d(9)-ee | 100 | 96 | 82 | 24 | 0.2 |
| 473589 | e5-d(10)-e5 | 50 | 93 | 116 | 22 | 0.2 |
| 529804 | k-d(10)-kekee | 50 | 54 | 74 | 27 | 0.2 |
| 534796 | ekk-d(10)-kke | 50 | 60 | 63 | 27 | 0.2 |
| 540162 | eek-d(10)-kke | 50 | 43 | 55 | 29 | 0.2 |
| 540175 | eek-d(10)-kke | 50 | 113 | 78 | 24 | 0.3 |
| 540182 | eek-d(10)-kke | 50 | 147 | 95 | 26 | 0.1 |
| 540191 | eek-d(10)-kke | 50 | 79 | 88 | 28 | 0.2 |

e = 2′-MOE, k = cEt, d = 2′-deoxynucleoside

### Body and Organ Weights

Body weights, as well as liver, heart, lungs, spleen and kidney weights were measured at the end of the study, and are presented in Table 44. Several of the ISIS oligonucleotides did not cause any changes in organ weights outside the expected range and were therefore deemed tolerable in terms of organ weights.

TABLE 44
Body and organ weights (grams) of CD-1 mice

| | | Dose | | | | |
|---|---|---|---|---|---|---|
| | | (mg/ | Body | Liv- | | Kid- |
| | Motif | kg/wk) | weight | er | Spleen | ney |
| PBS | — | — | 42 | 2.2 | 0.12 | 0.64 |
| 407935 | e5-d(10)-e5 | 100 | 40 | 2.6 | 0.20 | 0.62 |
| 490279 | kdkdk-d(9)-ee | 100 | 42 | 2.8 | 0.17 | 0.61 |
| 473589 | e5-d(10)-e5 | 50 | 41 | 2.5 | 0.16 | 0.67 |
| 529804 | k-d(10)-kekee | 50 | 40 | 2.3 | 0.14 | 0.62 |
| 534796 | ekk-d(10)-kke | 50 | 37 | 2.6 | 0.15 | 0.51 |
| 540162 | eek-d(10)-kke | 50 | 42 | 2.4 | 0.15 | 0.60 |
| 540175 | eek-d(10)-kke | 50 | 39 | 2.2 | 0.11 | 0.62 |
| 540182 | eek-d(10)-kke | 50 | 41 | 2.6 | 0.16 | 0.61 |
| 540191 | eek-d(10)-kke | 50 | 40 | 2.4 | 0.13 | 0.60 |

e = 2′-MOE, k = cEt, d = 2′-deoxynucleoside

### Example 35: Tolerability of Antisense Oligonucleotides Targeting Human Target-X in Sprague-Dawley Rats

Sprague-Dawley rats were treated with ISIS antisense oligonucleotides selected from studies described above and evaluated for changes in the levels of various plasma chemistry markers.

### Treatment

Two groups of 4 male 7-8 week old Sprague-Dawley rats each were injected subcutaneously twice a week for 6 weeks with 50 mg/kg of ISIS 407935 and ISIS 490279. Another seven groups of 4 male 6-8 week old Sprague-Dawley rats each were injected subcutaneously twice a week for 6 weeks with 25 mg/kg of ISIS 473589, ISIS 529804, ISIS 534796, ISIS 540162, ISIS 540175, ISIS 540182, and ISIS 540191. One group of male Sprague-Dawley rats was injected subcutaneously twice a week for 6 weeks with PBS. The rats were euthanized 48 hours after the last dose, and organs and plasma were harvested for further analysis.

### Plasma Chemistry Markers

To evaluate the effect of ISIS oligonucleotides on liver and kidney function, plasma levels of transaminases, bilirubin, albumin, and BUN were measured using an automated clinical chemistry analyzer (Hitachi Olympus AU400e, Melville, NY). The results are presented in Table 45. Treatment with the all antisense oligonucleotides was tolerable in terms of plasma chemistry markers in this model.

TABLE 45
Effect of antisense oligonucleotide treatment
on liver function in Sprague-Dawley rats

| | | Dose | | AST | BUN | Bilirubin |
|---|---|---|---|---|---|---|
| | Motif | (mg/kg/wk) | ALT(IU/L) | (IU/L) | (mg/dL) | (mg/dL) |
| PBS | — | — | 71 | 83 | 19 | 0.2 |
| 407935 | e5-d(10)-e5 | 100 | 74 | 96 | 22 | 0.2 |
| 490279 | kdkdk-d(9)-ee | 100 | 96 | 181 | 22 | 0.4 |
| 473589 | e5-d(10)-e5 | 50 | 57 | 73 | 21 | 0.2 |
| 529804 | k-d(10)-kekee | 50 | 54 | 78 | 21 | 0.2 |
| 534796 | ekk-d(10)-kke | 50 | 68 | 98 | 22 | 0.2 |
| 540162 | eek-d(10)-kke | 50 | 96 | 82 | 21 | 0.1 |
| 540175 | eek-d(10)-kke | 50 | 55 | 73 | 18 | 0.2 |
| 540182 | eek-d(10)-kke | 50 | 45 | 87 | 21 | 0.2 |
| 540191 | eek-d(10)-kke | 50 | 77 | 104 | 21 | 0.2 |

e = 2′-MOE, k = cEt, d = 2′-deoxynucleoside

### Body and Organ Weights

Body weights, as well as liver, heart, lungs, spleen and kidney weights were measured at the end of the study, and are presented in Table 46. Treatment with all the antisense oligonucleotides was tolerable in terms of body and organ weights in this model.

TABLE 46
Body and organ weights (grams) of Sprague-Dawley rats

| | | Dose | | | | |
|---|---|---|---|---|---|---|
| | | (mg/ | Body | Liv- | | Kid- |
| | Motif | kg/wk) | weight | er | Spleen | ney |
| PBS | — | — | 443 | 16 | 0.8 | 3.5 |
| ISIS 407935 | e5-d(10)-e5 | 100 | 337 | 14 | 1.8 | 3.2 |
| ISIS 490279 | kdkdk-d(9)-ee | 100 | 365 | 18 | 2.2 | 2.9 |
| ISIS 473589 | e5-d(10)-e5 | 50 | 432 | 18 | 1.3 | 3.3 |
| ISIS 529804 | k-d(10)-kekee | 50 | 429 | 18 | 2.2 | 3.4 |
| ISIS 534796 | ekk-d(10)-kke | 50 | 434 | 15 | 1.4 | 3.3 |
| ISIS 540162 | eek-d(10)-kke | 50 | 446 | 18 | 1.1 | 3.3 |
| ISIS 540175 | eek-d(10)-kke | 50 | 467 | 16 | 1.0 | 3.5 |
| ISIS 540182 | eek-d(10)-kke | 50 | 447 | 22 | 2.5 | 4.5 |
| ISIS 540191 | eek-d(10)-kke | 50 | 471 | 21 | 1.4 | 3.9 |

e = 2′-MOE, k = cEt, d = 2′-deoxynucleoside

### Example 36: Dose-Dependent Antisense Inhibition of Human Target-X in Cynomolgous Monkey Primary Hepatocytes

Antisense oligonucleotides selected from the studies described above were tested at various doses in cynomolgous monkey primary hepatocytes. Cells were plated at a density of 35,000 cells per well and transfected using electroporation with 0.009 μM, 0.03 μM, 0.08 μM, 0.25 μM, 0.74 μM, 2.22 μM, 6.67 μM, and 20.00 μM concentrations of antisense oligonucleotide, as specified in Table 47. After a treatment period of approximately 16 hours, RNA was isolated from the cells and Target-X mRNA levels were measured by quantitative real-time PCR. Target-X primer probe set RTS2927 was used to measure mRNA levels. Target-X mRNA levels were adjusted according to total RNA content, as measured by RIBOGREEN®. Results are presented as percent inhibition of Target-X, relative to untreated control cells. As illustrated in Table 47, Target-X mRNA levels were reduced in a dose-dependent manner with some of the antisense oligonucleotides that are cross-reactive with the rhesus monkey genomic sequence.

TABLE 47
Dose-dependent antisense inhibition of Target-X in cynomolgous
monkey primary hepatocytes using electroporation

| | 0.009 | 0.03 | 0.08 | 0.25 | 0.74 | 2.22 | 6.67 | 20.00 |
|---|---|---|---|---|---|---|---|---|
| ISIS No | μM | μM | μM | μM | μM | μM | μM | μM |
| 407935 | 10 | 18 | 15 | 29 | 56 | 73 | 82 | 88 |
| 490279 | 19 | 12 | 13 | 0 | 6 | 18 | 27 | 22 |
| 473589 | 5 | 10 | 19 | 42 | 64 | 76 | 88 | 92 |
| 529804 | 10 | 3 | 23 | 25 | 57 | 80 | 86 | 91 |
| 534796 | 0 | 28 | 23 | 49 | 71 | 81 | 87 | 90 |
| 540162 | 9 | 14 | 9 | 6 | 13 | 13 | 11 | 31 |
| 540175 | 0 | 4 | 12 | 9 | 10 | 16 | 12 | 22 |
| 540182 | 0 | 7 | 0 | 6 | 36 | 12 | 10 | 0 |
| 540191 | 6 | 7 | 0 | 0 | 0 | 0 | 21 | 42 |

### Example 37: Dose-Dependent Antisense Inhibition of Human Target-X in Hep3B Cells

Antisense oligonucleotides from the study described above were also tested at various doses in Hep3B cells. Cells were plated at a density of 20,000 cells per well and transfected using electroporation with 0.009 μM, 0.03 μM, 0.08 μM, 0.25 μM, 0.74 μM, 2.22 μM, 6.67 μM, and 20.00 μM concentrations of antisense oligonucleotide, as specified in Table 48. After a treatment period of approximately 16 hours, RNA was isolated from the cells and Target-X mRNA levels were measured by quantitative real-time PCR. Target-X primer probe set RTS2927 was used to measure mRNA levels. Target-X mRNA levels were adjusted according to total RNA content, as measured by RIBOGREEN®. Results are presented as percent inhibition of Target-X, relative to untreated control cells. As illustrated in Table 48, Target-X mRNA levels were reduced in a dose-dependent manner with several of the antisense oligonucleotides.

TABLE 48
Dose-dependent antisense inhibition of Target-X
in Hep3B cells using electroporation

| | 0.009 | 0.03 | 0.08 | 0.25 | 0.74 | 2.22 | 6.67 | 20.00 | IC₅₀ |
|---|---|---|---|---|---|---|---|---|---|
| ISIS No | μM | μM | μM | μM | μM | μM | μM | μM | (μM) |
| 407935 | 3 | 9 | 11 | 35 | 64 | 83 | 87 | 93 | 4.5 |
| 473244 | 20 | 33 | 50 | 69 | 77 | 89 | 7 | 14 | 0.9 |
| 473589 | 0 | 14 | 23 | 44 | 74 | 88 | 90 | 94 | 2.7 |
| 490279 | 0 | 5 | 7 | 15 | 25 | 61 | 76 | 78 | 11.6 |
| 515533 | 0 | 12 | 21 | 36 | 63 | 78 | 88 | 94 | 3.6 |
| 515952 | 0 | 12 | 27 | 57 | 76 | 89 | 93 | 94 | 2.2 |
| 516066 | 6 | 0 | 12 | 26 | 52 | 70 | 81 | 86 | 6.0 |
| 529459 | 0 | 4 | 24 | 40 | 61 | 78 | 88 | 94 | 3.5 |
| 529553 | 9 | 7 | 17 | 40 | 58 | 74 | 87 | 93 | 4.6 |
| 529804 | 0 | 3 | 34 | 64 | 83 | 89 | 93 | 95 | 2.0 |
| 534796 | 8 | 18 | 43 | 67 | 82 | 89 | 95 | 96 | 1.4 |
| 537806 | 6 | 11 | 5 | 20 | 37 | 69 | 79 | 86 | 7.1 |
| 540162 | 18 | 33 | 63 | 75 | 87 | 91 | 91 | 92 | 0.7 |
| 540175 | 10 | 25 | 55 | 76 | 86 | 89 | 89 | 93 | 1.0 |
| 540182 | 13 | 36 | 61 | 75 | 84 | 88 | 90 | 93 | 0.7 |
| 540191 | 3 | 12 | 28 | 61 | 79 | 80 | 88 | 94 | 2.2 |

### Example 38: Efficacy of Antisense Oligonucleotides Targeting Human Target-X in Transgenic Mice

Transgenic mice were treated with ISIS antisense oligonucleotides selected from studies described above and evaluated for efficacy.

### Treatment

Eight groups of 3 transgenic mice each were injected subcutaneously twice a week for 3 weeks with 20 mg/kg/week, 10 mg/kg/week, 5 mg/kg/week, or 2.5 mg/kg/week of ISIS 407935 or ISIS 490279. Another 24 groups of 3 transgenic mice each were subcutaneously twice a week for 3 weeks with 5 mg/kg/week, 2.5 mg/kg/week, 1.25 mg/kg/week, or 0.625 mg/kg/week of ISIS 473589, ISIS 529804, ISIS 534796, ISIS 540162, ISIS 540175, or ISIS 540191. One group of mice was injected subcutaneously twice a week for 3 weeks with PBS. Mice were euthanized 48 hours after the last dose, and organs and plasma were harvested for further analysis.

### RNA Analysis

RNA was extracted from plasma for real-time PCR analysis of Target-X, using primer probe set RTS2927. The mRNA levels were normalized using RIBOGREEN®. As shown in Table 49, several antisense oligonucleotides achieved reduction of human Target-X over the PBS control. Results are presented as percent inhibition of Target-X, relative to control. Treatment with newly designed 2′-MOE gapmer, ISIS 490279, caused greater reduction in human Target-X mRNA levels than treatment with ISIS 407935, the 2′-MOE gapmer from the earlier publication. Treatment with several of the newly designed oligonucleotides also caused greater reduction in human Target-X mRNA levels than treatment with ISIS 407935.

TABLE 49
Percent inhibition of Target-X mRNA in transgenic mice

| | | Dose | % |
|---|---|---|---|
| ISIS No | Motif | (mg/kg/wk) | inhibition |
| 407935 | e5-d(10)-e5 | 20.0 | 85 |
| | | 10.0 | 57 |
| | | 5.0 | 45 |
| | | 2.5 | 28 |
| 490279 | kdkdk-d(9)-ee | 20.0 | 88 |
| | | 10.0 | 70 |
| | | 5.0 | 51 |
| | | 2.5 | 33 |
| 473589 | e5-d(10)-e5 | 5.00 | 80 |
| | | 2.50 | 62 |
| | | 1.25 | 44 |
| | | 0.625 | 25 |
| 529804 | k-d(10)-kekee | 5.00 | 55 |
| | | 2.50 | 41 |
| | | 1.25 | 0 |
| | | 0.625 | 1 |
| 534796 | ekk-d(10)-kke | 5.00 | 56 |
| | | 2.50 | 41 |
| | | 1.25 | 5 |
| | | 0.625 | 0 |
| 540162 | eek-d(10)-kke | 5.00 | 97 |
| | | 2.50 | 92 |
| | | 1.25 | 69 |
| | | 0.625 | 78 |
| 540175 | eek-d(10)-kke | 5.00 | 95 |
| | | 2.50 | 85 |
| | | 1.25 | 65 |
| | | 0.625 | 55 |
| 540182 | eek-d(10)-kke | 5.00 | 97 |
| | | 2.50 | 83 |
| | | 1.25 | 54 |
| | | 0.625 | 10 |
| 540191 | eek-d(10)-kke | 5.00 | 91 |
| | | 2.50 | 74 |
| | | 1.25 | 58 |
| | | 0.625 | 34 |

e = 2′-MOE, k = cEt, d = 2′-deoxynucleoside

### Protein Analysis

Plasma protein levels of Target-X were estimated using a Target-X ELISA kit (purchased from Hyphen Bio-Med). As shown in Table 50, several antisense oligonucleotides achieved reduction of human Target-X over the PBS control. Results are presented as percent inhibition of Target-X, relative to control.

TABLE 50
Percent inhibition of Target-X plasm
protein levels in transgenic mice

| | | Dose | % |
|---|---|---|---|
| ISIS No | Motif | (mg/kg/wk) | inhibition |
| 407935 | e5-d(10)-e5 | 20 | 65 |
| | | 10 | 47 |
| | | 5 | 0 |
| | | 2.5 | 3 |
| 490279 | kdkdk-d(9)-ee | 20 | 91 |
| | | 10 | 75 |
| | | 5 | 31 |
| | | 2.5 | 23 |
| 473589 | e5-d(10)-e5 | 5 | 78 |
| | | 2.5 | 40 |
| | | 1.25 | 6 |
| | | 0.625 | 0 |
| 529804 | k-d(10)-kekee | 5 | 50 |
| | | 2.5 | 36 |
| | | 1.25 | 0 |
| | | 0.625 | 8 |
| 534796 | ekk-d(10)-kke | 5 | 45 |
| | | 2.5 | 26 |
| | | 1.25 | 0 |
| | | 0.625 | 8 |
| 540162 | eek-d(10)-kke | 5 | 98 |
| | | 2.5 | 96 |
| | | 1.25 | 78 |
| | | 0.625 | 74 |
| 540175 | eek-d(10)-kke | 5 | 93 |
| | | 2.5 | 83 |
| | | 1.25 | 49 |
| | | 0.625 | 24 |
| 540182 | eek-d(10)-kke | 5 | 97 |
| | | 2.5 | 71 |
| | | 1.25 | 50 |
| | | 0.625 | 0 |
| 540191 | eek-d(10)-kke | 5 | 97 |
| | | 2.5 | 74 |
| | | 1.25 | 46 |
| | | 0.625 | 25 |

e = 2′-MOE, k = cEt, d = 2′-deoxynucleoside

### Example 39: Effect of ISIS Antisense Oligonucleotides Targeting Human Target-X in Cynomolgus Monkeys

Cynomolgus monkeys were treated with ISIS antisense oligonucleotides selected from studies described above, including ISIS 407935, ISIS 490279, ISIS 473589, ISIS 529804, ISIS 534796, ISIS 540162, ISIS 540175, ISIS 540182, and ISIS 540191. Antisense oligonucleotide efficacy was evaluated. ISIS 407935, from the earlier publication, was included in the study for comparison.

### Treatment

Prior to the study, the monkeys were kept in quarantine for at least a 30-day period, during which the animals were observed daily for general health. Standard panels of serum chemistry and hematology, examination of fecal samples for ova and parasites, and a tuberculosis test were conducted immediately after the animals' arrival to the quarantine area. The monkeys were 2-4 years old at the start of treatment and weighed between 2 and 4 kg. Ten groups of four randomly assigned male cynomolgus monkeys each were injected subcutaneously with ISIS oligonucleotide or PBS using a stainless steel dosing needle and syringe of appropriate size into one of 4 sites on the back of the monkeys; each site used in clock-wise rotation per dose administered. Nine groups of monkeys were dosed four times a week for the first week (days 1, 3, 5, and 7) as loading doses, and subsequently once a week for weeks 2-12, with 35 mg/kg of ISIS 407935, ISIS 490279, ISIS 473589, ISIS 529804, ISIS 534796, ISIS 540162, ISIS 540175, ISIS 540182, or ISIS 540191. A control group of cynomolgus monkeys was injected with PBS subcutaneously thrice four times a week for the first week (days 1, 3, 5, and 7), and subsequently once a week for weeks 2-12. The protocols described in the Example were approved by the Institutional Animal Care and Use Committee (IACUC).

### Hepatic Target Reduction

### RNA Analysis

On day 86, RNA was extracted from liver tissue for real-time PCR analysis of Target-X using primer probe set RTS2927. Results are presented as percent inhibition of Target-X mRNA, relative to PBS control, normalized to RIBOGREEN® or to the house keeping gene, GAPDH. As shown in Table 52, treatment with ISIS antisense oligonucleotides resulted in reduction of Target-X mRNA in comparison to the PBS control.

TABLE 52
Percent Inhibition of cynomolgous monkey Target-X mRNA in
the cynomolgus monkey liver relative to the PBS control

| ISIS No | Motif | RTS2927/Ribogreen | RTS2927/GAPDH |
|---|---|---|---|
| 407935 | e5-d(10)-e5 | 90 | 90 |
| 490279 | kdkdk-d(9)-ee | 72 | 66 |
| 473589 | e5-d(10)-e5 | 96 | 96 |
| 529804 | k-d(10)-kekee | 90 | 87 |
| 534796 | ekk-d(10)-kke | 80 | 78 |
| 540162 | eek-d(10)-kke | 66 | 58 |
| 540175 | eek-d(10)-kke | 68 | 66 |
| 540182 | eek-d(10)-kke | 0 | 0 |
| 540191 | eek-d(10)-kke | 34 | 14 |

e = 2′-MOE, k = cEt, d = 2′-deoxynucleoside

### Protein Levels and Activity Analysis

Plasma Target-X levels were measured prior to dosing, and on day 3, day 5, day 7, day 16, day 30, day 44, day 65, and day 86 of treatment. Target-X activity was measured using Target-X deficient plasma. Approximately 1.5 mL of blood was collected from all available study animals into tubes containing 3.2% sodium citrate. The samples were placed on ice immediately after collection. Collected blood samples were processed to platelet poor plasma and the tubes were centrifuged at 3,000 rpm for 10 min at 4° C. to obtain plasma.

Protein levels of Target-X were measured by a Target-X elisa kit (purchased from Hyphen BioMed). The results are presented in Table 53.

TABLE 53
Plasma Target-X protein levels (% reduction compared
to the baseline) in the cynomolgus monkey plasma

| | Day | Day | Day | Day | Day | Day | Day | Day |
|---|---|---|---|---|---|---|---|---|
| ISIS No | 3 | 5 | 7 | 16 | 30 | 44 | 65 | 86 |
| 407935 | 21 | 62 | 69 | 82 | 84 | 85 | 84 | 90 |
| 490279 | 0 | 29 | 35 | 30 | 38 | 45 | 51 | 58 |
| 473589 | 12 | 67 | 85 | 97 | 98 | 98 | 98 | 98 |
| 529804 | 19 | 65 | 76 | 87 | 88 | 89 | 90 | 90 |
| 534796 | 1 | 46 | 54 | 64 | 64 | 67 | 66 | 70 |
| 540162 | 0 | 24 | 26 | 37 | 45 | 49 | 49 | 50 |
| 540175 | 0 | 28 | 36 | 38 | 47 | 52 | 55 | 55 |
| 540182 | 0 | 17 | 8 | 0 | 0 | 0 | 5 | 0 |
| 540191 | 0 | 12 | 4 | 0 | 0 | 4 | 9 | 10 |

### Example 40

### Single Nucleotide Polymorphisms (SNPs) in the Huntingtin (HTT) Gene Sequence

SNP positions (identified by Hayden et al, WO/2009/135322) associated with the HTT gene were mapped to the HTT genomic sequence, designated herein as SEQ ID NO: 1 (NT 006081.18 truncated from nucleotides 1566000 to 1768000). Table 56 provides SNP positions associated with the HTT gene. Table 56 provides a reference SNP ID number from the Entrez SNP database at the National Center for Biotechnology Information (NCBI, http://www.ncbi.nlm.nih.gov/sites/entrez?db=snp), incorporated herein by reference. Table 56 furnishes further details on each SNP. The ‘Reference SNP ID number’ or ‘RS number’ is the number designated to each SNP from the Entrez SNP database at NCBI, incorporated herein by reference. ‘SNP position’ refers to the nucleotide position of the SNP on SEQ ID NO: 1. ‘Polymorphism’ indicates the nucleotide variants at that SNP position. ‘Major allele’ indicates the nucleotide associated with the major allele, or the nucleotide present in a statistically significant proportion of individuals in the human population. ‘Minor allele’ indicates the nucleotide associated with the minor allele, or the nucleotide present in a relatively small proportion of individuals in the human population.

TABLE 56
Single Nuclear Polymorphisms (SNPs)
and their positions on SEQ ID NO: 1

| | SNP | | Major | Minor |
|---|---|---|---|---|
| RS No. | position | Polymorphism | allele | allele |
| rs2857936 | 1963 | C/T | C | T |
| rs12506200 | 3707 | A/G | G | A |
| rs762855 | 14449 | A/G | G | A |
| rs3856973 | 19826 | G/A | G | A |
| rs2285086 | 28912 | G/A | A | G |
| rs7659144 | 37974 | C/G | C | G |
| rs16843804 | 44043 | C/T | C | T |
| rs2024115 | 44221 | G/A | A | G |
| rs10015979 | 49095 | A/G | A | G |
| rs7691627 | 51063 | A/G | G | A |
| rs2798235 | 54485 | G/A | G | A |
| rs4690072 | 62160 | G/T | T | G |
| rs6446723 | 66466 | C/T | T | C |
| rs363081 | 73280 | G/A | G | A |
| rs363080 | 73564 | T/C | C | T |
| rs363075 | 77327 | G/A | G | A |
| rs363064 | 81063 | T/C | C | T |
| rs3025849 | 83420 | A/G | A | G |
| rs6855981 | 87929 | A/G | G | A |
| rs363102 | 88669 | G/A | A | G |
| rs11731237 | 91466 | C/T | C | T |
| rs4690073 | 99803 | A/G | G | A |
| rs363144 | 100948 | T/G | T | G |
| rs3025838 | 101099 | C/T | C | T |
| rs34315806 | 101687 | A/G | G | A |
| rs363099 | 101709 | T/C | C | T |
| rs363096 | 119674 | T/C | T | C |
| rs2298967 | 125400 | C/T | T | C |
| rs2298969 | 125897 | A/G | G | A |
| rs6844859 | 130139 | C/T | T | C |
| rs363092 | 135682 | C/A | C | A |
| rs7685686 | 146795 | A/G | A | G |
| rs363088 | 149983 | A/T | A | T |
| rs362331 | 155488 | C/T | T | C |
| rs916171 | 156468 | G/C | C | G |
| rs362322 | 161018 | A/G | A | G |
| rs362275 | 164255 | T/C | C | T |
| rs362273 | 167080 | A/G | A | G |
| rs2276881 | 171314 | G/A | G | A |
| rs3121419 | 171910 | T/C | C | T |
| rs362272 | 174633 | G/A | G | A |
| rs362271 | 175171 | G/A | G | A |
| rs3775061 | 178407 | C/T | C | T |
| rs362310 | 179429 | A/G | G | A |
| rs362307 | 181498 | T/C | C | T |
| rs362306 | 181753 | G/A | G | A |
| rs362303 | 181960 | T/C | C | T |
| rs362296 | 186660 | C/A | C | A |
| rs1006798 | 198026 | A/G | A | G |

### Example 41

### Modified Oligonucleotides Targeting Huntingtin (HTT) Single Nucleotide Polymorphism (SNP)

A series of modified oligonucleotides were designed based on the parent gapmer, ISIS 460209 wherein the central gap region contains nine 2′-deoxyribonucleosides. These modified oligonucleotides were designed by introducing various chemical modifications in the central gap region and were tested for their ability to selectively inhibit mutant (mut) HTT mRNA expression levels targeting rs7685686 while leaving the expression of the wild-type (wt) intact. The activity and selectivity of the modified oligonucleotides were evaluated and compared to the parent gapmer, ISIS 460209.

The modified oligonucleotides were created with a 3-9-3 motif and are described in Table 57. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e”, “k”, “y”, or “z” are sugar modified nucleosides. A subscript “e” indicates a 2′-O-methoxyethyl (MOE) modified nucleoside, a subscript “k” indicates a 6′-(S)—CH₃ bicyclic nucleoside (e.g. cEt), a subscript “y” indicates an α-L-LNA bicyclic nucleoside and a subscript “z” indicates a F-HNA modified nucleoside. ^{p}U indicates a 5-propyne uridine nucleoside and ^{x}T indicates a 2-thio-thymidine nucleoside.

The number in parentheses indicates the position on the modified oligonucleotide opposite to the SNP position, as counted from the 5′-terminus.

### Cell Culture and Transfection

The modified oligonucleotides were tested in vitro. Heterozygous fibroblast GM04022 cell line was used (from Coriell Institute). Cultured GM04022 cells at a density of 25,000 cells per well were transfected using electroporation with 0.12, 0.37, 1.1, 3.3 and 10 μM concentrations of modified oligonucleotides. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. The HTT mRNA levels were adjusted according to total RNA content, as measured by RIBOGREEN and the results are presented below.

### Analysis of IC₅₀'s

The half maximal inhibitory concentration (IC₅₀) of each oligonucleotide is presented in Table 58 and was calculated by plotting the concentrations of oligonucleotides used versus the percent inhibition of HTT mRNA expression achieved at each concentration, and noting the concentration of oligonucleotide at which 50% inhibition of HTT mRNA expression was achieved compared to the control. The IC₅₀ at which each oligonucleotide inhibits the mutant HTT mRNA expression is denoted as ‘mut IC₅₀’. The IC₅₀ at which each oligonucleotide inhibits the wild-type HTT mRNA expression is denoted as ‘wt IC₅₀’. Selectivity was calculated by dividing the IC₅₀ for inhibition of the wild-type HTT versus the IC₅₀ for inhibiting expression of the mutant HTT mRNA.

The parent gapmer, ISIS 460209 is marked with an asterisk (*) in the table and was included in the study as a benchmark oligonucleotide against which the activity and selectivity of the modified oligonucleotides targeting nucleotides overlapping the SNP position could be compared.

As illustrated in Table 58, modified oligonucleotides having chemical modifications in the central gap region at the SNP position exhibited similar activity with an increase in selectivity comparing to the parent gapmer, wherein the central gap region contains full deoxyribonucleosides.

TABLE 57
Modified oligonucleotides targeting HTT rs7685686

| | | | Wing | | SEQ |
|---|---|---|---|---|---|
| ISIS | | Gap | chemistry | | ID |
| NO | Sequence (5′ to 3′) | chemistry | 5′ | 3′ | NO |
| 460209* (8) | TₑAₖAₖATTGTCATCAₖCₖCₑ | Full Deoxy | ekk | kke | 10 |
| 539560 (8) | TₑAₖAₖATTG^{p}UCATCAₖCₖCₑ | Deoxy/5-Propyne | ekk | kke | 11 |
| 539563 (8) | TₑAₖAₖATTG^{x}TCATCAₖCₖCₑ | Deoxy/2-Thio | ekk | kke | 10 |
| 539554 (8) | TₑAₖAₖATTGU_{y}CATCAₖCₖCₑ | Deoxy/α-L-LNA | ekk | kke | 11 |
| 542686 (8) | TₑAₖAₖATTGT_{z}CATCAₖCₖCₑ | Deoxy/F-HNA | ekk | kke | 10 |

e = 2′-MOE, k = cEt

TABLE 58
Comparison of inhibition of HTT mRNA levels and selectivity of modified
oligonucleotides with ISIS 460209 targeted to rs7685686 in GM04022 cells

| ISIS | Mut IC₅₀ | Wt IC₅₀ | Selectivity | | Wing chemistry | |
|---|---|---|---|---|---|---|
| NO | (μM) | (μM) | (mut vs wt) | Gap chemistry | 5′ | 3′ |
| 460209* (8) | 0.41 | 2.0 | 4.9 | Full Deoxy | ekk | kke |
| 539560 (8) | 0.29 | 1.1 | 3.8 | Deoxy/5-Propyne | ekk | kke |
| 539563 (8) | 0.45 | 3.1 | 6.9 | Deoxy/2-Thio | ekk | kke |
| 539554 (8) | 3.5 | >10 | >3 | Deoxy/α-L-LNA | ekk | kke |
| 542686 (8) | 0.5 | 3.1 | 6.0 | Deoxy/F-HNA | ekk | kke |

### Example 42

### Modified Oligonucleotides Comprising Chemical Modifications in the Gap Region Targeting Huntingtin (HTT) Single Nucleotide Polymorphism (SNP)

Additional modified oligonucleotides were designed in a similar manner as the antisense oligonucleotides described in Table 57. Various chemical modifications were introduced in the central gap region at the SNP position in an effort to improve selectivity while maintaining activity in reducing mutant HTT mRNA levels.

The modified oligonucleotides were created with a 3-9-3 motif and are described in Table 59. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “a”, “e”, “f”, “h”, “k”, “1”, “R”, “w” are sugar modified nucleosides. A subscript “a” indicates a 2′-(ara)-F modified nucleoside, a subscript “e” indicates a 2′-O-methoxyethyl (MOE) modified nucleoside, a subscript “f” indicates a 2′-F modified nucleoside, a subscript “h” indicates a HNA modified nucleoside, a subscript “k” indicates a 6′-(S)—CH₃ bicyclic nucleoside (e.g. cEt), a subscript “1” indicates a LNA modified nucleoside, a subscript “R” indicates a 5′-(R)-Me DNA, a subscript “w” indicates an unlocked nucleic acid (UNA) modified nucleoside. ⁿT indicates an N3-ethylcyano thymidine nucleoside and ^{b}N indicates an abasic nucleoside (e.g. 2′-deoxyribonucleoside comprising a H in place of a nucleobase). Underlined nucleoside or the number in parentheses indicates the position on the modified oligonucleotide opposite to the SNP position, as counted from the 5′-terminus.

### Thermal Stability Assay

The modified oligonucleotides were evaluated in thermal stability (Tₘ) assay. The Tₘ's were measured using the method described herein. A Cary 100 Bio spectrophotometer with the Cary Win UV Thermal program was used to measure absorbance vs. temperature. For the Tₘ experiments, oligonucleotides were prepared at a concentration of 8 μM in a buffer of 100 mM Na+, 10 mM phosphate, 0.1 mM EDTA, pH 7. Concentration of oligonucleotides were determined at 85° C. The oligonucleotide concentration was 4 μM with mixing of equal volumes of test oligonucleotide and mutant or wild-type RNA strand. Oligonucleotides were hybridized with the mutant or wild-type RNA strand by heating duplex to 90° C. for 5 min and allowed to cool at room temperature. Using the spectrophotometer, Tₘ measurements were taken by heating duplex solution at a rate of 0.5 C/min in cuvette starting @ 15° C. and heating to 85° C. Tₘ values were determined using Vant Hoff calculations (A₂₆₀ vs temperature curve) using non self-complementary sequences where the minimum absorbance which relates to the duplex and the maximum absorbance which relates to the non-duplex single strand are manually integrated into the program.

Presented in Table 60 is the Tₘ for the modified oligonucleotides when duplexed to mutant or wild-type RNA complement. The Tₘ of the modified oligonucleotides duplexed with mutant RNA complement is denoted as “Tₘ (° C.) mut”. The Tₘ of the modified oligonucleotides duplexed with wild-type RNA complement is denoted as “Tₘ (° C.) wt”.

### Cell Culture, Transfection and Selectivity Analysis

The modified oligonucleotides were also tested in vitro. Heterozygous fibroblast GM04022 cell line was used. Cultured GM04022 cells at a density of 25,000 cells per well were transfected using electroporation with a single dose at 2 μM concentration of the modified oligonucleotide. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. The HTT mRNA levels were adjusted according to total RNA content, as measured by RIBOGREEN. The results in Table 60 are presented as percent of HTT mRNA expression, relative to untreated control levels and is denoted as “% UTC”. Selectivity as was also evaluated and measured by dividing the percent of wild-type HTT mRNA levels vs. the percent of mutant HTT mRNA levels.

The parent gapmer, ISIS 460209 is marked with an asterisk (*) in the table and was included in the study as a benchmark oligonucleotide against which the selectivity of the modified oligonucleotides targeting nucleotides overlapping the SNP position could be compared.

As illustrated in Table 60, improvement in selectivity was observed for antisense oligonucleotides comprising chemical modifications in the central gap region at the SNP site such as 5′-(R)-Me (ISIS 539558), HNA (ISIS 539559), and 2′-(ara)-F (ISIS 539565) in comparison to the parent full deoxy gapmer, ISIS 460209. Modified oligonucleotides comprising LNA (ISIS 539553) or 2′-F (ISIS 539570) showed comparable selectivity while UNA modification (ISIS 539556 or 543909) showed no selectivity. Modified oligonucleotides comprising modified nucleobase, N3-ethylcyano (ISIS 539564) or abasic nucleobase (ISIS 543525) showed little to no improvement in selectivity.

TABLE 59
Modified oligonucleotides comprising chemical modifications
in the central gap region

| | | | Wing | | SEQ |
|---|---|---|---|---|---|
| ISIS | | | chemistry | | ID |
| NO | Sequence (5′ to 3′) | Gap chemistry | 5′ | 3′ | NO. |
| 460209* (8) | TₑAₖAₖATTGTCATCAₖCₖCₑ | Full Deoxy | ekk | kke | 10 |
| 539553 (8) | TₑAₖAₖATTGTₗCATCAₖCₖCₑ | Deoxy/LNA | ekk | kke | 10 |
| 539556 (8) | TₑAₖAₖATTGU_{w}CATCAₖCₖCₑ | Deoxy/UNA | ekk | kke | 11 |
| 539558 (8) | TₑAₖAₖATTGT_{R}CATCAₖCₖCₑ | Deoxy/5′-(R)-Me DNA | ekk | kke | 10 |
| 539559 (8) | TₑAₖAₖATTGTₕCATCAₖCₖCₑ | Deoxy/HNA | ekk | kke | 10 |
| 539564 (8) | TₑAₖAₖATTGⁿTCATCAₖCₖCₑ | Deoxy/deoxy with N3- | ekk | kke | 10 |
| | | Ethylcyano nucleobase | | | |
| 539565 (8) | TₑAₖAₖATTGTₐCATCAₖCₖCₑ | Deoxy/2′-(ara)-F | ekk | kke | 10 |
| 539570 (8) | TₑAₖAₖATTGT_{f}CATCAₖCₖCₑ | Deoxy/2′-F | ekk | kke | 10 |
| 543525 (8) | TₑAₖAₖATTG^{b}NCATCAₖCₖCₑ | Deoxy/Deoxy-Abasic | ekk | kke | 12 |
| 543909 (5) | TₑAₖAₖAU_{w}TGTCATCAₖCₖCₑ | Deoxy/UNA | ekk | kke | 13 |

e = 2′-MOE, k = cEt, d = 2′-deoxyribonucleoside

TABLE 60
Comparison of selectivity in inhibition of HTT mRNA levels and Tm of modified
oligonucleotides with ISIS 460209 targeted to rs7685686 in GM04022 cells

| | | | | | | | Wing | |
|---|---|---|---|---|---|---|---|---|
| ISIS | Tm (° C.) | | % UTC | | Selectivity | | chemistry | |
| NO | mutant | wt | mutant | wt | (wt vs mut) | Gap chemistry | 5′ | 3′ |
| 460209* (8) | 53.7 | 52.2 | 23 | 57 | 2.4 | Full Deoxy | ekk | kke |
| 539553 (8) | 57.7 | 55.3 | 54 | 102 | 1.9 | Deoxy/LNA | ekk | kke |
| 539556 (8) | 43.7 | 44.1 | 90 | 105 | 1.2 | Deoxy/UNA | ekk | kke |
| 539558 (8) | 51.2 | 49.7 | 25 | 83 | 3.3 | Deoxy/5′-(R)-Me DNA | ekk | kke |
| 539559 (8) | 55.4 | 50.5 | 18 | 62 | 3.5 | Deoxy/HNA | ekk | kke |
| 539564 (8) | 42.8 | 43.1 | 86 | 135 | 1.6 | Deoxy/Deoxy N3- | ekk | kke |
| | | | | | | ethylcyano nucleobase | | |
| 539565 (8) | 53.8 | 52.5 | 14 | 46 | 3.4 | Deoxy/2′-(ara)-F | ekk | kke |
| 539570 (8) | 54.4 | 51.8 | 25 | 50 | 2.0 | Deoxy/2′-F | ekk | kke |
| 543525 (8) | 43.1 | 43.8 | 87 | 97 | 1.1 | Deoxy/Deoxy Abasic | ekk | kke |
| 543909 (5) | 44.7 | 42.1 | 68 | 79 | 1.2 | Deoxy/UNA | ekk | kke |

e = 2′-MOE, k = cEt, d = 2′-deoxyribonucleoside

### Example 43

### Chimeric Oligonucleotides Comprising Self-Complementary Regions Targeting Huntingtin (HTT) Single Nucleotide Polymorphism (SNP)

Chimeric oligonucleotides were designed based on the parent gapmer, ISIS 460209. These gapmers comprise self-complementary regions flanking the central gap region, wherein the central gap region contains nine deoxyribonucleosides and the self-complementary regions are complementary to one another. The underlined nucleosides indicate the portion of the 5′-end that is self-complement to the portion of the 3′-end.

The gapmers and their motifs are described in Table 61. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e” or “k” are sugar modified nucleosides. A subscript “e” indicates a 2′-O-methoxyethyl (MOE) modified nucleoside and a subscript “k” indicates a 6′-(S)—CH₃ bicyclic nucleoside (e.g. cEt).

The modified oligonucleotides were tested in vitro. Heterozygous fibroblast GM04022 cell line was used. Cultured GM04022 cells at a density of 25,000 cells per well were transfected using electroporation with a single dose at 2 μM concentration of the modified oligonucleotide. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. HTT mRNA levels were adjusted according to total RNA content, as measured by RIBOGREEN. The results in Table 62 are presented as percent of HTT mRNA expression, relative to untreated control levels and is denoted as “% UTC”. Selectivity was also evaluated and measured by dividing the percent of wild-type HTT mRNA levels vs. the percent of the mutant HTT mRNA levels.

The parent gapmer, ISIS 460209 is marked with an asterisk (*) in the table and was included in the study as a benchmark oligonucleotide against which the selectivity of the modified oligonucleotides targeting nucleotides overlapping the SNP position could be compared.

As illustrated in Table 62, improvement in selectivity was observed for chimeric oligonucleotides comprising 5-9-5 (ISIS 550913), 6-9-6 (ISIS 550912), 6-9-3 (ISIS 550907) or 3-9-7 (ISIS 550904) in comparison to the parent gapmer motif, 3-9-3 (ISIS 460209). The remaining gapmers showed moderate to little improvement in selectivity.

TABLE 61
Chimeric oligonucleotides comprising various wing motifs
targeted to HTT rs7685686

| | | | Wing | | SEQ |
|---|---|---|---|---|---|
| ISIS | | | chemistry | | ID |
| NO | Sequence (5′ to 3′) | Motif | 5′ | 3′ | NO. |
| 460209* | TₑAₖAₖATTGTCATCAₖCₖCₑ | 3-9-3 | ekk | kke | 10 |
| 544838 | TₑAₖAₖATTGTCATCAₖCₖCₑAₖ | 3-9-4 | ekk | kkek | 14 |
| 544840 | TₑAₖAₖATTGTCATCAₖCₖCₑTₖTₖAₖ | 3-9-6 | ekk | kkekkk | 15 |
| 544842 | TₑAₖAₖATTGTCATCAₖCₖCₑAₖTₖTₖTₖAₖ | 3-9-8 | ekk | kkekkkkk | 16 |
| 550903 | TₑAₖAₖATTGTCATCAₖCₖCₑTₖAₖ | 3-9-5 | ekk | kkekk | 17 |
| 550904 | TₑAₖAₖATTGTCATCAₖCₖCₑTₖTₖTₖAₖ | 3-9-7 | ekk | kkekkkk | 18 |
| 550905 | GₖTₑAₖAₖATTGTCATCAₖCₖCₑ | 4-9-3 | kekk | kke | 19 |
| 550906 | GₖGₖTₑAₖAₖATTGTCATCAₖCₖCₑ | 5-9-3 | kkekk | kke | 20 |
| 550907 | GₖGₖTₖTₑAₖAₖATTGTCATCAₖGₖCₑ | 6-9-3 | kkkekk | kke | 21 |
| 550908 | GₖGₖTₖGₖTₑAₖAₖATTGTCATCAₖCₖCₑ | 7-9-3 | kkkkekk | kke | 22 |
| 550909 | GₖGₖTₖGₖAₖTₑAₖAₖATTGTCATCAₖCₖCₑ | 8-9-3 | kkkkkekk | kke | 23 |
| 550910 | GₖGₖCₖTₑAₖAₖATTGTCATCAₖCₖCₑGₖCₖCₖ | 6-9-6 | kkkekk | kkekkk | 24 |
| 550911 | GₖCₖTₑAₖAₖATTGTCATCAₖCₖCₑGₖCₖ | 5-9-5 | kkekk | kkekk | 25 |
| 550912 | TₖAₖAₖTₑAₖAₖATTGTCATCAₖCₖCₑTₖTₖAₖ | 6-9-6 | kkkekk | kkekkk | 26 |
| 550913 | AₖAₖTₑAₖAₖATTGTCATCAₖCₖCₑTₖTₖ | 5-9-5 | kkekk | kkekk | 27 |
| 550914 | TₖCₖTₖTₑAₖAₖATTGTCATCAₖCₖCₑAₖGₖAₖ | 6-9-6 | kkkekk | kkekkk | 28 |
| 550915 | CₖTₖTₑAₖAₖATTGTCATCAₖCₖCₑAₖGₖ | 5-9-5 | kkekk | kkekk | 29 |

e = 2′-MOE, k = cEt

TABLE 62
Comparison of selectivity in inhibition of HTT mRNA
levels of chimeric oligonucleotides with ISIS 460209
targeted to rs7685686 in GM04022 cells

| ISIS | % UTC | | Selectivity | | wing chemistry | |
|---|---|---|---|---|---|---|
| NO | mut | wt | (wt vs. mut) | Motif | 5′ | 3′ |
| 460209* | 23 | 57 | 2.4 | 3-9-3 | ekk | kke |
| 544838 | 13 | 25 | 2.0 | 3-9-4 | ekk | kkek |
| 544840 | 17 | 31 | 1.8 | 3-9-6 | ekk | kkekkk |
| 544842 | 55 | 102 | 1.9 | 3-9-8 | ekk | kkekkkkk |
| 550903 | 13 | 36 | 2.7 | 3-9-5 | ekk | kkekk |
| 550904 | 23 | 67 | 3.0 | 3-9-7 | ekk | kkekkkk |
| 550905 | 21 | 51 | 2.4 | 4-9-3 | kekk | kke |
| 550906 | 23 | 67 | 2.9 | 5-9-3 | kkekk | kke |
| 550907 | 30 | 93 | 3.1 | 6-9-3 | kkkekk | kke |
| 550908 | 60 | 80 | 2.4 | 7-9-3 | kkkkekk | kke |
| 550909 | 42 | 101 | 2.4 | 8-9-3 | kkkkkekk | kke |
| 550910 | 57 | 102 | 1.8 | 6-9-6 | kkkekk | kkekkk |
| 550911 | 18 | 40 | 2.2 | 5-9-5 | kkekk | kkekk |
| 550912 | 14 | 51 | 3.6 | 6-9-6 | kkkekk | kkekkk |
| 550913 | 8 | 36 | 4.5 | 5-9-5 | kkekk | kkekk |
| 550914 | 29 | 45 | 1.5 | 6-9-6 | kkkekk | kkekkk |
| 550915 | 13 | 28 | 2.1 | 5-9-5 | kkekk | kkekk |

e = 2′-MOE, k = cEt

### Example 44

### Chimeric Antisense Oligonucleotides Comprising Non-Self-Complementary Regions Targeting Huntingtin (HTT) Single Nucleotide Polymorphism (SNP)

Additional gapmers are designed based on the most selective gapmers from studies described in Tables 61 and 62 (ISIS 550912 and 550913). These gapmers are created such that they cannot form self-structure in the effort to evaluate if the increased activity simply is due to higher binding affinity. Gapmers are designed by deleting two or three nucleotides at the 3′-terminus and are created with 6-9-3 or 5-9-3 motif.

The chimeric oligonucleotides and their motifs are described in Table 63. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e” or “k” are sugar modified nucleosides. A subscript “e” indicates a 2′-O-methoxyethyl (MOE) modified nucleoside and a subscript “k” indicates a 6′-(S)—CH₃ bicyclic nucleoside (e.g. cEt).

The gapmers, ISIS 550912 and ISIS 550913, from which the newly designed gapmers are derived from, are marked with an asterisk (*) in the table.

TABLE 63
Non-self-complementary chimeric oligonucleotides
targeting HTT SNP

| | | | Wing | | SEQ |
|---|---|---|---|---|---|
| ISIS | | | chemistry | | ID |
| NO | Sequence (5′ to 3′) | Motif | 5′ | 3′ | NO. |
| 550912* | TₖAₖAₖTₑAₖAₖATTGTCATCAₖCₖCₑTₖTₖAₖ | 6-9-6 | kkkekk | kkekkk | 26 |
| 550913* | AₖAₖTₑAₖAₖATTGTCATCAₖCₖCₑTₖTₖ | 5-9-5 | kkekk | kkekk | 27 |
| 556879 | TₖAₖAₖTₑAₖAₖATTGTCATCAₖCₖCₑ | 6-9-3 | kkkekk | kke | 30 |
| 556880 | AₖAₖTₑAₖAₖATTGTCATCAₖCₖCₑ | 5-9-3 | kkekk | kke | 31 |

e = 2′-MOE, k = cEt

### Example 45

### Chimeric Oligonucleotides Containing Mismatches Targeting Huntingtin (HTT) Single Nucleotide Polymorphism (SNP)

A series of chimeric antisense oligonucleotides were designed based on the parent gapmer, ISIS 460209, wherein the central gap region contains nine 2′-deoxyribonucleosides. These gapmers were designed by introducing modified nucleosides at both 5′ and 3′ termini. Gapmers were also created with a single mismatch shifted slightly upstream and downstream (i.e. “microwalk”) within the central gap region and with the SNP position opposite position 5 of the parent gapmer, as counted from the 5′-gap terminus.

The gapmers and their motifs are described in Table 64. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e” or “k” are sugar modified nucleosides. A subscript “e” indicates a 2′-O-methoxyethyl (MOE) modified nucleoside and a subscript “k” indicates a 6′-(S)—CH₃ bicyclic nucleoside (e.g. cEt). Underlined nucleosides indicate the mismatch position, as counted from the 5′-gap terminus.

These gapmers were evaluated for thermal stability (Tₘ) using methods described in Example 42. Presented in Table 65 are the Tₘ measurements for chimeric antisense oligonucleotides when duplexed to mutant or wild-type RNA complement. The Tₘ of chimeric antisense oligonucleotides duplexed with mutant RNA complement is denoted as “Tₘ (° C.) mut”. The Tₘ of chimeric antisense oligonucleotides duplexed with wild-type RNA complement is denoted as “Tₘ (° C.) wt”.

These gapmers were also tested in vitro. Heterozygous fibroblast GM04022 cell line was used. Cultured GM04022 cells at a density of 25,000 cells per well were transfected using electroporation with a single dose at 2 μM concentration of the modified oligonucleotide. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. HTT mRNA levels were adjusted according to total RNA content, as measured by RIBOGREEN. The results in Table 65 are presented as percent of HTT mRNA expression, relative to untreated control levels and is denoted as “% UTC”. Selectivity was also evaluated and measured by dividing the percent of wild-type HTT mRNA levels vs. the percent of mutant HTT mRNA levels.

The parent gapmer, ISIS 460209 is marked with an asterisk (*) in the table and was included in the study as a benchmark oligonucleotide against which the selectivity of the modified oligonucleotides targeting nucleotides overlapping the SNP position could be compared.

As illustrated in Table 65, improvement in selectivity was observed for gapmers comprising a 4-9-4 motif with a central deoxy gap region (ISIS 476333) or a single mismatch at position 8 within the gap region (ISIS 543531) in comparison to the parent gapmer. The remaining gapmers showed moderate to little improvement in selectivity.

TABLE 64
Chimeric oligonucleotides containing a single
mismatch targeting mutant HTT SNP

| | | | | Wing | | SEQ |
|---|---|---|---|---|---|---|
| ISIS | | Mismatch | | chemistry | | ID |
| NO | Sequence (5′ to 3′) | position | Motif | 5′ | 3′ | NO. |
| 460209* | TₑAₖAₖATTGTCATCAₖCₖCₑ | — | 3-9-3 | ekk | kke | 10 |
| 476333 | AₑTₖAₑAₖATTGTCATCAₖCₑCₖAₑ | — | 4-9-4 | ekek | keke | 32 |
| 543526 | AₑTₖAₑAₖATTCTCATCAₖCₑCₖAₑ | 4 | 4-9-4 | ekek | keke | 33 |
| 543527 | AₑTₖAₑAₖATAGTCATCAₖCₑCₖAₑ | 3 | 4-9-4 | ekek | keke | 34 |
| 543529 | AₑTₖAₑAₖATTGTGATCAₖCₑCₖAₑ | 6 | 4-9-4 | ekek | keke | 35 |
| 543530 | AₑTₖAₑAₖATTGTCTTCAₖCₑCₖAₑ | 7 | 4-9-4 | ekek | keke | 36 |
| 543531 | AₑTₖAₑAₖATTGTCAACAₖCₑCₖAₑ | 8 | 4-9-4 | ekk | keke | 37 |
| 543532 | TₑAₖAₖATTCTCATCAₖCₖCₑ | 4 | 3-9-3 | ekk | kke | 38 |
| 543534 | TₑAₖAₖAATGTCATCAₖCₖCₑ | 2 | 3-9-3 | ekk | kke | 39 |
| 543535 | TₑAₖAₖATTGTGATCAₖCₖCₑ | 6 | 3-9-3 | ekk | kke | 40 |
| 543536 | TₑAₖAₖATTGTCTTCAₖCₖCₑ | 7 | 3-9-3 | ekk | kke | 41 |
| 543537 | TₑAₖAₖATTGTCAACAₖCₖCₑ | 8 | 3-9-3 | ekk | kke | 42 |

e = 2′-MOE, k = cEt

TABLE 65
Comparison of selectivity and Tₘ of chimeric oligonucleotides
with ISIS 460209 targeted to rs7685686 in GM04022 cells

| ISIS | Tm (° C.) | | % UTC | | Selectivity | Mismatch | | Wing chemistry | |
|---|---|---|---|---|---|---|---|---|---|
| NO | mut | wt | mut | wt | (wt vs mut) | position | Motif | 5′ | 3′ |
| 460209* | 53.7 | 52.2 | 23 | 57 | 2.4 | — | 3-9-3 | ekk | kke |
| 476333 | 60.2 | 58.4 | 10 | 37 | 3.6 | — | 4-9-4 | ekek | keke |
| 543526 | 47.9 | 46.6 | 70 | 86 | 1.2 | 4 | 4-9-4 | ekek | keke |
| 543527 | 52.6 | 49.9 | 40 | 103 | 2.6 | 3 | 4-9-4 | ekek | keke |
| 543529 | 50.3 | 49.0 | 66 | 102 | 1.5 | 6 | 4-9-4 | ekek | keke |
| 543530 | 52.9 | 50.9 | 67 | 110 | 1.6 | 7 | 4-9-4 | ekek | keke |
| 543531 | 53.3 | 50.3 | 46 | 136 | 3.0 | 8 | 4-9-4 | ekk | keke |
| 543532 | 43.6 | 42.8 | 127 | 151 | 1.2 | 4 | 3-9-3 | ekk | kke |
| 543534 | 45.9 | 43.8 | 67 | 95 | 1.4 | 2 | 3-9-3 | ekk | kke |
| 543535 | 44.0 | 43.3 | 96 | 113 | 1.2 | 6 | 3-9-3 | ekk | kke |
| 543536 | 46.8 | 44.6 | 106 | 104 | 1.0 | 7 | 3-9-3 | ekk | kke |
| 543537 | 45.9 | 44.3 | 77 | 81 | 1.1 | 8 | 3-9-3 | ekk | kke |

e = 2′-MOE, k = cEt

### Example 46

### Chimeric Oligonucleotides Comprising Mismatches Targeting Huntingtin (HTT) Single Nucleotide Polymorphism (SNP)

Additional chimeric antisense oligonucleotides are designed based on two gapmers selected from studies described in Tables 64 and 65 (ISIS 476333 and ISIS 460209) wherein the central gap region contains nine 2′-deoxyribonucleosides. These gapmers are designed by introducing a single mismatch, wherein the mismatch will be shifted throughout the antisense oligonucleotide (i.e. “microwalk”). Gapmers are also created with 4-9-4 or 3-9-3 motifs and with the SNP position opposite position 8 of the original gapmers, as counted from the 5′-terminus.

The gapmers and their motifs are described in Table 66. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e” or “k” are sugar modified nucleosides. A subscript “e” indicates a 2′-O-methoxyethyl (MOE) modified nucleoside and a subscript “k” indicates a 6′-(S)—CH₃ bicyclic nucleoside (e.g. cEt). Underlined nucleosides indicate the mismatch position, as counted from the 5′-terminus.

The gapmers, ISIS 476333 and ISIS 460209, in which the newly designed antisense oligonucleotides are derived from, are marked with an asterisk (*) in the table.

TABLE 66
Chimeric oligonucleotides comprising mismatches
targeting HTT SNP

| | | | | Wing | | SEQ |
|---|---|---|---|---|---|---|
| ISIS | | Mismatch | | chemistry | | ID |
| NO | Sequence (5′ to 3′) | position | Motif | 5′ | 3′ | NO |
| 476333* | AₑTₖAₑAₖATTGTCATCAₖCₑCₖAₑ | — | 4-9-4 | ekek | keke | 32 |
| 554209 | TₑTₖAₑAₖATTGTCATCAₖCₑCₖAₑ | 1 | 4-9-4 | ekek | keke | 43 |
| 554210 | AₑAₖAₑAₖATTGTCATCAₖCₑCₖAₑ | 2 | 4-9-4 | ekek | keke | 44 |
| 554211 | AₑTₖTₑAₖATTGTCATCAₖCₑCₖAₑ | 3 | 4-9-4 | ekek | keke | 45 |
| 554212 | AₑTₖAₑTₖATTGTCATCAₖCₑCₖAₑ | 4 | 4-9-4 | ekek | keke | 46 |
| 554213 | AₑTₖAₑAₖTTTGTCATCAₖCₑCₖAₑ | 5 | 4-9-4 | ekek | keke | 47 |
| 554214 | AₑTₖAₑAₖATTGTCATGAₖCₑCₖAₑ | 13 | 4-9-4 | ekek | keke | 48 |
| 554215 | AₑTₖAₑAₖATTGTCATCTₖCₑCₖAₑ | 14 | 4-9-4 | ekek | keke | 49 |
| 554216 | AₑTₖAₑAₖATTGTCATCAₖGₑCₖAₑ | 15 | 4-9-4 | ekek | keke | 50 |
| 554217 | AₑTₖAₑAₖATTGTCATCAₖCₑGₖAₑ | 16 | 4-9-4 | ekek | keke | 51 |
| 554218 | AₑTₖAₑAₖATTGTCATCAₖCₑCₖTₑ | 17 | 4-9-4 | ekek | keke | 52 |
| 460209* | TₑAₖAₖATTGTCATCAₖCₖCₑ | — | 3-9-3 | ekk | kke | 10 |
| 562481 | TₑAₖAₖGTTGTCATCAₖCₖCₑ | 4 | 3-9-3 | ekk | kke | 53 |
| 554482 | TₑAₖAₖAGTGTCATCAₖCₖCₑ | 5 | 3-9-3 | ekk | kke | 54 |
| 554283 | TₑAₖAₖATGGTCATCAₖCₖCₑ | 6 | 3-9-3 | ekk | kke | 55 |

e = 2′-MOE, k = cEt

### Example 47

### Short-Gap Chimeric Oligonucleotides Targeting Huntingtin (HTT) Single Nucleotide Polymorphism (SNP)

Chimeric antisense oligonucleotides were designed based on the parent gapmer, ISIS 460209, wherein the central gap region contains nine 2′-deoxyribonucleosides. These gapmers were designed by shortening the central gap region to seven 2′-deoxyribonucleosides. Gapmers were also created with 5-7-5 motif and with the SNP position opposite position 8 or 9 of the parent gapmer, as counted from the 5′-terminus.

The gapmers and their motifs are described in Table 67. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e” or “k” are sugar modified nucleosides. A subscript “e” indicates a 2′-O-methoxyethyl (MOE) modified nucleoside and a subscript “k” indicates a 6′-(S)—CH₃ bicyclic nucleoside (e.g. cEt). Underlined nucleoside or the number in parentheses indicates the position on the modified oligonucleotide opposite to the SNP position, as counted from the 5′-terminus.

The chimeric antisense oligonucleotides were tested in vitro. ISIS 141923 was included in the study as a negative control and is denoted as “neg control”. A non-allele specific antisense oligonucleotide, ISIS 387916 was used as a positive control and is denoted as “pos control”. ISIS 460209 was included in the study for comparison. Heterozygous fibroblast GM04022 cell line was used. Cultured GM04022 cells at a density of 25,000 cells per well were transfected using electroporation with 0.12, 0.37, 1.1, 3.3, and 10 μM concentration of the modified oligonucleotide. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. HTT mRNA levels were adjusted according to total RNA content, as measured by RIBOGREEN and the results are presented in Table 68.

The IC₅₀ and selectivity were calculated using methods described previously in Example 41. As illustrated in Table 68, no improvement in potency and selectivity was observed for the chimeric antisense oligonucleotides as compared to ISIS 460209.

TABLE 67
Chimeric antisense oligonucleotides targeting HTT rs7685686

| | | | Wing | | SEQ |
|---|---|---|---|---|---|
| ISIS | | | Chemistry | | ID |
| NO | Sequence (5′ to 3′) | Motif | 5′ | 3′ | NO. |
| 460209* (8) | TₑAₖAₖATTGTCATCAₖCₖCₑ | 3-9-3 | ekk | kke | 10 |
| 460085 (9) | AₑTₑAₑAₑAₑTTGTCATCₑAₑCₑCₑAₑ | 5-7-5 | eeeee | eeeee | 32 |
| 540108 (9) | AₑTₑAₑAₖAₖTTGTCATCₖAₖCₑCₑAₑ | 5-7-5 | eeekk | kkeee | 32 |
| 387916 | TₑCₑTₑCₑTₑATTGCACATTCₑCₑAₑAₑGₑ | 5-10-5 | eeeee | eeeee | 56 |
| (pos control) | | | | | |
| 141923 | CₑCₑTₑTₑCₑCCTGAAGGTTCₑCₑTₑCₑCₑ | 5-10-5 | eeeee | eeeee | 57 |
| (neg control) | | | | | |

e = 2′-MOE, k = cEt

TABLE 68
Comparison of inhibition of HTT mRNA levels and selectivity
of chimeric antisense oligonucleotides with ISIS 460209
targeted to rs7685686 in GM04022 cells

| | | | | | Wing | |
|---|---|---|---|---|---|---|
| | Mut IC₅₀ | Wt IC₅₀ | Selectivity | | chemistry | |
| ISIS NO | (μM) | (μM) | (mut vs wt) | Motif | 5′ | 3′ |
| 460209* (8) | 0.41 | 2.0 | 4.9 | 3-9-3 | ekk | kke |
| 460085 (9) | 3.5 | >10 | >3 | 5-7-5 | eeeee | eeeee |
| 540108 (9) | 0.41 | — | — | 5-7-5 | eeekk | kkeee |
| 387916 | 0.39 | 0.34 | 1.0 | 5-10-5 | eeeee | eeeee |
| (pos control) | | | | | | |
| 141923 | >10 | >10 | — | 5-10-5 | eeeee | eeeee |
| (neg control) | | | | | | |

e = 2′-MOE,
k = cEt

### Example 48

### Short-Gap Chimeric Oligonucleotides Targeting Huntingtin (HTT) Single Nucleotide Polymorphism (SNP)

Additional chimeric antisense oligonucleotides were designed based on the parent gapmer, ISIS 460209, wherein the central gap region contains nine 2′-deoxyribonucleosides. These gapmers were designed with the central gap region shortened or interrupted by introducing various modifications either within the gap or by adding one or more modified nucleosides to the 3′-most 5′-region or to the 5′-most 3′-region. Gapmers were created with the SNP position opposite position 8 of the parent gapmer, as counted from the 5′-terminus.

The gapmers and their motifs are described in Table 69. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e” or “k” are sugar modified nucleosides. A subscript “e” indicates a 2′-O-methoxyethyl (MOE) modified nucleoside and a subscript “k” indicates a 6′-(S)—CH₃ bicyclic nucleoside (e.g. cEt).

The chimeric antisense oligonucleotides were tested in vitro. Heterozygous fibroblast GM04022 cell line was used. Cultured GM04022 cells at a density of 25,000 cells per well were transfected using electroporation with 2 μM concentration of the modified oligonucleotide. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. HTT mRNA levels were adjusted according to total RNA content, as measured by RIBOGREEN. The results in Table 70 are presented as percent of HTT mRNA expression, relative to untreated control levels and is denoted as “% UTC”. Selectivity was also evaluated and measured by dividing the percent of wild-type HTT mRNA levels vs. the percent of mutant HTT mRNA levels. ISIS 460209 marked with an asterisk (*) in the table was included in the study for comparison.

As illustrated in Table 70, modifications to the 3′-most 5′-region nucleosides that shorten the gap from 9 to 7 or 8 nucleotides (ISIS 551429 and ISIS 551426) improved selectivity and potency comparing to the parent gapmer (ISIS 460209). The remaining chimeric antisense oligonucleotides showed moderate to little improvement in selectivity.

TABLE 69
Short-gap antisense oligonucleotides targeting
HTT rs7685686

| | | | Wing | | SEQ |
|---|---|---|---|---|---|
| ISIS | | | Chemistry | | ID |
| NO | Sequence (5′ to 3′) | Motif | 5′ | 3′ | NO. |
| 460209* | TₑAₖAₖATTGTCATCAₖCₖCₑ | 3-9-3 | ekk | kke | 10 |
| 551426 | TₑAₖAₑAₖTTGTCATCAₖCₖCₑ | 4-8-3 | ekek | kke | 10 |
| 551427 | TₑAₖAₑATₖTGTCATCAₖCₖCₑ | 3-9-3 or | eke or | kke | 10 |
| | | 5-7-3 | ekedk | | |
| 551428 | TₑAₖAₑATTₖGTCATCAₖCₖCₑ | 3-9-3 or | eke or | kke | 10 |
| | | 6-6-3 | ekeddk | | |
| 551429 | TₑAₑAₑAₖTₖTGTCATCAₖCₖCₑ | 5-7-3 | eeekk | kke | 10 |

e = 2′-MOE, k = cEt, d = 2′-deoxyribonucleoside

TABLE 70
Comparison of selectivity in inhition of HTT mRNA
levels of antisense oligonucleotides with ISIS 460209
targeted to rs7685686 in GM4022 cells

| | % UTC | | Selectivity | | Wing chemistry | |
|---|---|---|---|---|---|---|
| ISIS NO | mut | wt | (wt vs. mut) | Motif | 5′ | 3′ |
| 460209* | 23 | 57 | 2.4 | 3-9-3 | ekk | kke |
| 551426 | 14 | 66 | 4.8 | 4-8-3 | ekek | kke |
| 551427 | 35 | 97 | 2.8 | 3-9-3 or | eke or | kke |
| | | | | 5-7-3 | ekedk | |
| 551428 | 61 | 110 | 1.8 | 3-9-3 or | eke or | kke |
| | | | | 6-6-3 | ekeddk | |
| 551429 | 19 | 94 | 5.0 | 5-7-3 | eeekk | kke |

e = 2′-MOE,
k = cEt,
d = 2′-deoxyribonucleoside

### Example 49

### Modified Oligonucleotides Targeting HTT SNP

A series of modified antisense oligonucleotides are designed based on the parent gapmer, ISIS 460209, wherein the central gap region contains nine 2′-deoxynucleosides and is marked with an asterisk (*) in the table. These modified oligonucleotides are designed by shortening or interrupting the gap with a single mismatch or various chemical modifications within the central gap region. The modified oligonucleotides are created with the SNP position opposite position 8 of the parent gapmer, as counted from the 5′-terminus.

The gapmers and their motifs are described in Table 71. The internucleoside linkages throughout each gapmer are phosphorothioate (P═S) linkages, except for the internucleoside linkage with a subscript “p”, “pz” or “pw”. Subscript “p” indicates methyl phosphonate internucleoside linkage. Subscript “pz” indicates (R)-methyl phosphonate internucleoside linkage. Subscript “pw” indicates (S)-methyl phosphonate internucleoside linkage. All cytosine nucleobases throughout each gapmer are 5-methyl cytosines. ^{x}T indicates a 2-thio thymidine nucleoside. Nucleosides without a subscript are β-D-2′-deoxyribonucleosides. Nucleosides followed by a subscript “e”, “k” or “b” are sugar modified nucleosides. A subscript “e” indicates a 2′-O-methoxyethyl (MOE) modified nucleoside, a subscript “k” indicates a 6′-(S)—CH₃ bicyclic nucleoside (e.g. cEt) and a subscript “b” indicates a 5′-Me DNA modified nucleoside. Underlined nucleosides indicate the position of modification. Bold and underlined nucleosides indicate the mismatch position.

TABLE 71
Short-gap chimeric oligonucleotides targeting
HTT SNP

| | | | | Wing | | SEQ |
|---|---|---|---|---|---|---|
| ISIS | Sequence | | Gap | Chemistry | | ID |
| NO | (5′ to 3′) | Motif | Chemistry | 5′ | 3′ | NO. |
| 460209* | TₑAₖAₖATTGTC | 3-9-3 | — | ekk | kke | 10 |
| | ATCAₖCₖCₑ | | | | | |
| XXXX16 | TₑAₖAₖA^{x}TTGT | 3-9-3 | Deoxy/2-thio | ekk | kke | 10 |
| | CATCAₖCₖCₑ | | | | | |
| XXXX17 | TₑAₖAₖAT^{x}TGT | 3-9-3 | Deoxy/2-thio | ekk | kke | 10 |
| | CATCAₖCₖCₑ | | | | | |
| XXXX18 | TₑAₖAₖA^{x}T^{x}TGT | 3-9-3 | Deoxy/2-thio | ekk | kke | 10 |
| | CATCAₖCₖCₑ | | | | | |
| XXXX19 | TₑAₖAₖATTₚGT | 3-9-3 | Deoxy/Methyl | ekk | kke | 10 |
| (558257) | CATCAₖCₖCₑ | | phosphonate | | | |
| XXXX20 | TₑAₖAₖATₚTGT | 3-9-3 | Deoxy/Methyl | ekk | kke | 10 |
| (558256) | CATCAₖCₖCₑ | | phosphonate | | | |
| XXXX20a | TₑAₖAₖAT_{pz}TGT | 3-9-3 | Deoxy/(R)- | ekk | kke | 10 |
| | CATCAₖCₖCₑ | | Methyl | | | |
| | | | phosphonate | | | |
| XXXX20b | TₑAₖAₖAT_{pw}TG | 3-9-3 | Deoxy/(S)- | ekk | kke | 10 |
| | TCATCAₖCₖCₑ | | Methyl | | | |
| | | | phosphonate | | | |
| XXXX21 | TₑAₖAₖAₚTTGT | 3-9-3 | Methyl | ekk | kke | 10 |
| (558255) | CATCAₖCₖCₑ | | phosphonate | | | |
| XXXX22 | TₑAₖAₖATT_{b}GT | 3-9-3 | 5′-Me-DNA | ekk | kke | 10 |
| | CATCAₖCₖCₑ | | | | | |
| XXXX23 | TₑAₖAₖAT_{b}TGT | 3-9-3 | 5′-Me-DNA | ekk | kke | 10 |
| | CATCAₖCₖCₑ | | | | | |
| XXXX24 | TₑAₖAₖA_{b}TTGT | 3-9-3 | 5′-Me-DNA | ekk | kke | 10 |
| | CATCAₖCₖCₑ | | | | | |
| XXXX25 | TₑAₖAₖGTTGTC | 4-8-3 | Mismatch at | ekk | kke | 53 |
| | ATCAₖCₖCₑ | | position 4 | | | |
| XXXX26 | TₑAₖAₖAGTGT | 5-7-3 | Mismatch at | ekk | kke | 54 |
| | CATCAₖCₖCₑ | | position 5 | | | |
| XXXX27 | TₑAₖAₖATGGT | 6-6-3 | Mismatch at | ekk | kke | 55 |
| | CATCAₖCₖCₑ | | position 6 | | | |

e = 2′-MOE, k = cEt

### Example 50

### Short-Gap Chimeric Oligonucleotides Comprising Modifications at the Wing Regions Targeting Huntingtin (HTT) Single Nucleotide Polymorphism (SNP)

Additional chimeric antisense oligonucleotides were designed based on the parent gapmer, ISIS 460209, wherein the central gap region contains nine 2′-deoxynucleosides. These gapmers were designed by shortening the central gap region to seven 2′-deoxynucleosides and introducing various modifications at the wing regions.

The gapmers and their motifs are described in Table 72. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e” or “k” are sugar modified nucleosides. A subscript “e” indicates a 2′-O-methoxyethyl (MOE) modified nucleoside and a subscript “k” indicates a 6′-(S)—CH₃ bicyclic nucleoside (e.g. cEt).

The number in parentheses indicates the position on the chimeric oligonucleotide opposite to the SNP position, as counted from the 5′-terminus.

These gapmers were evaluated for thermal stability (Tₘ) using methods described in Example 42. Presented in Table 73 is the Tₘ measurements for chimeric antisense oligonucleotides when duplexed to mutant or wild-type RNA complement. The Tₘ of chimeric antisense oligonucleotides duplexed with mutant RNA complement is denoted as “Tₘ (° C.) mut”. The Tₘ of chimeric antisense oligonucleotides duplexed with wild-type RNA complement is denoted as “Tₘ (° C.) wt”.

These gapmers were also tested in vitro. Heterozygous fibroblast GM04022 cell line was used. Cultured GM04022 cells at a density of 25,000 cells per well were transfected using electroporation with a single dose at 2 μM concentration of the modified oligonucleotide. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. HTT mRNA levels were adjusted according to total RNA content, as measured by RIBOGREEN. The results in Table 73 are presented as percent of HTT mRNA expression, relative to untreated control levels and is denoted as “% UTC”. Selectivity was also evaluated and measured by dividing the percent of wild-type HTT mRNA levels vs. the percent of mutant HTT mRNA levels. ISIS 460209 marked with an asterisk (*) in the table was included in the study for comparison.

As illustrated in Table 73, improvement in selectivity was observed for gapmers comprising 2-7-8 or 5-7-5 motifs having cEt subunits at the wing regions in comparison to the parent gapmer, ISIS 460209. The remaining gapmers showed moderate to little improvement in selectivity.

TABLE 72
Short-gap chimeric oligonucleotides comprising
wing modifications

| | | | wing | | SEQ |
|---|---|---|---|---|---|
| ISIS | | | chemistry | | ID |
| NO | Sequence (5′ to 3′) | Motif | 5′ | 3′ | NO. |
| 460209* (8) | TₑAₖAₖATTGTCATCAₖCₖCₑ | 3-9-3 | ekk | kke | 10 |
| 540103 (6) | AₖAₖTTGTCATCₑAₑCₑCₑAₑGₑAₑAₑ | 2-7-8 | kk | e8 | 58 |
| 540104 (6) | AₑAₑTTGTCATCₑAₑCₑCₑAₑGₑAₑAₑ | 2-7-8 | ee | e8 | 59 |
| 540105 (7) | AₑAₑAₑTTGTCATCₑAₑCₑCₑAₑGₑAₑ | 3-7-7 | eee | e7 | 60 |
| 540106 (8) | TₑAₑAₑAₑTTGTCATCₑAₑCₑCₑAₑGₑ | 4-7-6 | eeee | e6 | 61 |
| 540107 (9) | AₑTₑAₑAₑAₖTTGTCATCₖAₑCₑCₑAₑ | 5-7-5 | eeeek | keeee | 32 |
| 540109 (10) | AₑAₑTₑAₑAₑAₑTTGTCATCₑAₑCₑCₑ | 6-7-4 | e6 | e4 | 62 |
| 540110 (11) | TₑAₑAₑTₑAₑAₑAₑTTGTCATCₑAₑCₑ | 7-7-3 | e7 | eee | 63 |
| 540111 (12) | TₑTₑAₑAₑTₑAₑAₑAₑTTGTCATCₑAₑ | 8-7-2 | e8 | ee | 64 |
| 540112 (12) | TₑTₑAₑAₑTₑAₑAₑAₑTTGTCATCₖAₖ | 8-7-2 | e8 | kk | 64 |

e = 2′-MOE (e.g. e6 = eeeeee), and k = cEt

TABLE 73
Comparison of selectivity in inhibition of HTT mRNA levels of
antisense oligonucleotides with ISIS 460209 targeted to
RS7685686 in GM04022 cells

| | | | | | Selec- | | | |
|---|---|---|---|---|---|---|---|---|
| | Tm | | | | tivity | | wing | |
| | (° C.) | | % UTC | | (wt vs | | chemistry | |
| ISIS NO | mut | wt | mut | wt | mut) | Motif | 5′ | 3′ |
| 460209* (8) | 53.7 | 52.2 | 23 | 57 | 2.4 | 3-9-3 | ekk | kke |
| 540103 (6) | 57.6 | 56.4 | 23 | 74 | 3.3 | 2-7-8 | kk | e8 |
| 540104 (6) | 54.8 | 52.8 | 36 | 91 | 2.5 | 2-7-8 | ee | e8 |
| 540105 (7) | 54.2 | 52.2 | 53 | 135 | 2.6 | 3-7-7 | eee | e7 |
| 540106 (8) | 52.4 | 50.8 | 30 | 77 | 2.6 | 4-7-6 | eeee | e6 |
| 540107 (9) | 56.6 | 54.7 | 19 | 62 | 3.3 | 5-7-5 | eeeek | keeee |
| 540109 (10) | 49.1 | 47.3 | 78 | 127 | 1.6 | 6-7-4 | e6 | e4 |
| 540110 (11) | 42.8 | 41.2 | 89 | 112 | 1.3 | 7-7-3 | e7 | eee |
| 540111 (12) | 39.0 | 36.9 | 111 | 128 | 1.1 | 8-7-2 | e8 | ee |
| 540112 (12) | 44.2 | 42.4 | 86 | 102 | 1.2 | 8-7-2 | e8 | kk |

### Example 51

Chimeric Oligonucleotides with SNP Site Shifting within the Central Gap Region

Chimeric antisense oligonucleotides were designed based on the parent gapmer, ISIS 460209 wherein the SNP site aligns with position 5 of the parent gapmer, as counted from the 5′-gap terminus. These gapmers were designed by shifting the SNP site upstream or downstream (i.e. microwalk) within the central gap region of the parent gapmer.

The gapmers and their motifs are described in Table 74. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e” or “k” are sugar modified nucleosides. A subscript “e” indicates a 2′-O-methoxyethyl (MOE) modified nucleoside and a subscript “k” indicates a 6′-(S)—CH₃ bicyclic nucleoside (e.g. cEt). Underline nucleosides indicate the position on the chimeric oligonucleotide aligns with the SNP site.

The SNP site indicates the position on the chimeric antisense oligonucleotide opposite to the SNP position, as counted from the 5′-gap terminus and is denoted as “SNP site”.

The chimeric oligonucleotides were tested in vitro. Heterozygous fibroblast GM04022 cell line was used. Cultured GM04022 cells at a density of 25,000 cells per well were transfected using electroporation with 0.12, 0.37, 1.1, 3.3 and 10 μM concentrations of modified oligonucleotides. After a treatment period of approximately 16 hours, RNA was isolated from the cells and mRNA levels were measured by quantitative real-time PCR using ABI assay C_2229297_10 which measures at dbSNP rs362303. The HTT mRNA levels were adjusted according to total RNA content, as measured by RIBOGREEN. ISIS 460209 marked with an asterisk (*) in the table was included in the study for comparison.

The IC₅₀ and selectivity were calculated using the methods previously described in Example 41. As illustrated in Table 75, chimeric oligonucleotides comprising 4-9-2 (ISIS 540082) or 2-9-4 (ISIS 540095) motif with the SNP site at position 1 or 3 showed comparable activity and 2.5 fold selectivity as compared to their counterparts.

TABLE 74
Chimeric oligonucleotides designed by microwalk

| | | | | wing | | SEQ |
|---|---|---|---|---|---|---|
| ISIS | | | SNP | chemistry | | ID |
| NO | Sequence (5′ to 3′) | Motif | site | 5′ | 3′ | NO. |
| 460209* | TₑAₖAₖATTGTCATCAₖCₖCₑ | 3-9-3 | 5 | ekk | kke | 10 |
| 540082 | AₑTₖTₖGₖTCATCACCAGₖAₑ | 4-9-2 | 1 | ekkk | ke | 65 |
| 540089 | TₑTₖAₖAₖTAAATTGTCAₖTₑ | 4-9-2 | 8 | ekkk | ke | 66 |
| 540095 | AₑTₖTGTCATCACCₖAₖGₖAₑ | 2-9-4 | 3 | ek | kkke | 65 |

e = 2′-MOE, and k = cEt

TABLE 75
Comparison of inhibition of HTT mRNA levels and
selectivity of chimeric oligonucleotides with ISIS
460209 targeted to HTT SNP

| | Mut | | | | | Wing | |
|---|---|---|---|---|---|---|---|
| | IC₅₀ | Wt IC₅₀ | Selectivity | | SNP | Chemistry | |
| ISIS NO | (μM) | (μM) | (wt vs mut) | Motif | site | 5′ | 3′ |
| 460209 | 0.41 | 2.0 | 4.9 | 3-9-3 | 5 | ekk | kke |
| 540082 | 0.45 | 5.6 | 12 | 4-9-2 | 1 | ekkk | ke |
| 540089 | >10 | >10 | — | 4-9-2 | 8 | ekkk | ke |
| 540095 | 0.69 | 8.4 | 12 | 2-9-4 | 3 | ek | kkke |

e = 2′-MOE, and
k = cEt

### Example 52

Chimeric Oligonucleotides with SNP Site Shifting at Various Positions

Chimeric antisense oligonucleotides were designed based on the parent gapmer, ISIS 460209 wherein the SNP site aligns with position 8 of the parent gapmer, as counted from the 5′-terminus. These gapmers were designed by shifting the SNP site upstream or downstream (i.e. microwalk) of the original oligonucleotide.

The gapmers and their motifs are described in Table 76. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e” or “k” are sugar modified nucleosides. A subscript “e” indicates a 2′-O-methoxyethyl (MOE) modified nucleoside and a subscript “k” indicates a 6′-(S)—CH₃ bicyclic nucleoside (e.g. cEt). Underline nucleosides indicate the SNP site.

The SNP site indicates the position on the chimeric antisense oligonucleotide opposite to the SNP position, as counted from the 5′-terminus and is denoted as “SNP site”.

The chimeric oligonucleotides were tested in vitro. Heterozygous fibroblast GM04022 cell line was used. Cultured GM04022 cells at a density of 25,000 cells per well were transfected using electroporation with 0.12, 0.37, 1.1, 3.3 and 10 μM concentrations of modified oligonucleotides. After a treatment period of approximately 16 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. HTT mRNA levels were adjusted according to total RNA content, as measured by RIBOGREEN. The results in Table 77 are presented as percent of HTT mRNA expression, relative to untreated control levels and is denoted as “% UTC”. Selectivity was also evaluated and measured by dividing the percent of wild-type HTT mRNA levels vs. the percent of mutant HTT mRNA levels.

The parent gapmer, ISIS 460209 is marked with an asterisk (*) in the table and was included in the study as a benchmark oligonucleotide against which the selectivity of the modified oligonucleotides targeting nucleotides overlapping the SNP position could be compared.

As illustrated in Table 77, improvement in potency and selectivity was observed for chimeric oligonucleotides comprising 4-9-2 or 2-9-4 motif having the target SNP site at positions 3, 4, 6, 7 and 8 (ISIS540083, ISIS540084, ISIS 540085, ISIS 540094, ISIS 540096, ISIS 540097 and ISIS 540098) in comparison to position 8 of the parent gapmer (ISIS 460209). The remaining gapmers showed little to no improvement in potency or selectivity.

TABLE 76
Chimeric oligonucleotides designed by
microwalk

| | | | | SEQ |
|---|---|---|---|---|
| ISIS | | SNP | | ID |
| NO | Sequence (5′ to 3′) | site | Motif | NO. |
| 460209* | TₑAₖAₖATTGTCATCAₖCₖCₑ | 8 | 3-9-3 | 10 |
| | | | (ekk- | |
| | | | d9- | |
| | | | kke) | |
| 543887 | TₑTₖGₖTₖCATCACCAGAₖAₑ | 4 | 4-9-2 | 67 |
| | | | (ekkk- | |
| | | | d9-ke) | |
| 540083 | AₑAₖTₖTₖGTCATCACCAₖGₑ | 6 | 4-9-2 | 68 |
| | | | (ekkk- | |
| | | | d9-ke) | |
| 540084 | AₑAₖAₖTₖTGTCATCACCₖAₑ | 7 | 4-9-2 | 69 |
| | | | (ekkk- | |
| | | | d9-ke) | |
| 540085 | TₑAₖAₖAₖTTGTCATCACₖCₑ | 8 | 4-9-2 | 10 |
| | | | (ekkk- | |
| | | | d9-ke) | |
| 540087 | AₑAₖTₖAₖAATTGTCATCₖAₑ | 10 | 4-9-2 | 70 |
| | | | (ekkk- | |
| | | | d9-ke) | |
| 540090 | AₑTₖTₖAₖATAAATTGTCₖAₑ | 13 | 4-9-2 | 71 |
| | | | (ekkk- | |
| | | | d9-ke) | |
| 540091 | TₑAₖTₖTₖAATAAATTGTₖCₑ | 14 | 4-9-2 | 72 |
| | | | (ekkk- | |
| | | | d9-ke) | |
| 540092 | GₑTₖCATCACCAGAₖAₖAₖAₑ | 2 | 2-9-4 | 73 |
| | | | (ek- | |
| | | | d9- | |
| | | | kkke) | |
| 540093 | TₑGₖTCATCACCAGₖAₖAₖAₑ | 3 | 2-9-4 | 74 |
| | | | (ek- | |
| | | | d9- | |
| | | | kkke) | |
| 540094 | TₑTₖGTCATCACCAₖGₖAₖAₑ | 4 | 2-9-4 | 67 |
| | | | (ek- | |
| | | | d9- | |
| | | | kkke) | |
| 540096 | AₑAₖTTGTCATCACₖCₖAₖGₑ | 6 | 2-9-4 | 68 |
| | | | (ek- | |
| | | | d9- | |
| | | | kkke) | |
| 540097 | AₑAₖATTGTCATCAₖCₖCₖAₑ | 8 | 2-9-4 | 69 |
| | | | (ek- | |
| | | | d9- | |
| | | | kkke) | |
| 540098 | TₑAₖAATTGTCATCₖAₖCₖCₑ | 8 | 2-9-4 | 10 |
| | | | (ek- | |
| | | | d9- | |
| | | | kkke) | |
| 540099 | AₑTₖAAATTGTCATₖCₖAₖCₑ | 9 | 2-9-4 | 75 |
| | | | (ek- | |
| | | | d9- | |
| | | | kkke) | |
| 540100 | AₑAₖTAAATTGTCAₖTₖCₖAₑ | 10 | 2-9-4 | 70 |
| | | | (ek- | |
| | | | d9- | |
| | | | kkke) | |
| 540101 | TₑAₖATAAATTGTCₖAₖTₖCₑ | 11 | 2-9-4 | 76 |
| | | | (ek- | |
| | | | d9- | |
| | | | kkke) | |
| 540102 | TₑTₖAATAAATTGTₖCₖAₖTₑ | 12 | 2-9-4 | 66 |
| | | | (ek- | |
| | | | d9- | |
| | | | kkke) | |

e = 2′-MOE; k = cEt; d = 2′-deoxyribonucleoside

TABLE 77
Comparison of selectivity in HTT SNP inhibition
of chimeric oligonucleotides with ISIS 460209

| | % UTC | | Selectivity | SNP | |
|---|---|---|---|---|---|
| ISIS NO | mut | wt | (wt vs. mut) | site | Motif |
| 460209* | 23 | 57 | 2.4 | 8 | 3-9-3 |
| | | | | | (ekk-d9-kke) |
| 543887 | 18 | 43 | 2.3 | 4 | 4-9-2 |
| | | | | | (ekkk-d9-ke) |
| 540083 | 18 | 67 | 3.7 | 6 | 4-9-2 |
| | | | | | (ekkk-d9-ke) |
| 540084 | 10 | 49 | 4.9 | 7 | 4-9-2 |
| | | | | | (ekkk-d9-ke) |
| 540085 | 21 | 86 | 4.1 | 8 | 4-9-2 |
| | | | | | (ekkk-d9-ke) |
| 540087 | 60 | 98 | 1.6 | 10 | 4-9-2 |
| | | | | | (ekkk-d9-ke) |
| 540090 | 129 | 137 | 1.1 | 13 | 4-9-2 |
| | | | | | (ekkk-d9-ke) |
| 540091 | 93 | 105 | 1.1 | 14 | 4-9-2 |
| | | | | | (ekkk-d9-ke) |
| 540092 | 28 | 55 | 2.0 | 2 | 2-9-4 |
| | | | | | (ek-d9-kkke) |
| 540093 | 18 | 62 | 3.4 | 3 | 2-9-4 |
| | | | | | (ek-d9-kkke) |
| 540094 | 13 | 45 | 3.4 | 4 | 2-9-4 |
| | | | | | (ek-d9-kkke) |
| 540096 | 17 | 68 | 4.0 | 6 | 2-9-4 |
| | | | | | (ek-d9-kkke) |
| 540097 | 8 | 35 | 4.2 | 8 | 2-9-4 |
| | | | | | (ek-d9-kkke) |
| 540098 | 12 | 45 | 3.9 | 8 | 2-9-4 |
| | | | | | (ek-d9-kkke) |
| 540099 | 62 | 91 | 1.5 | 9 | 2-9-4 |
| | | | | | (ek-d9-kkke) |
| 540100 | 80 | 106 | 1.3 | 10 | 2-9-4 |
| | | | | | (ek-d9-kkke) |
| 540101 | 154 | 152 | 1.0 | 11 | 2-9-4 |
| | | | | | (ek-d9-kkke) |
| 540102 | 102 | 106 | 1.0 | 12 | 2-9-4 |
| | | | | | (ek-d9-kkke) |

e = 2′-MOE; k = cEt; d = 2′-deoxyribonucleoside

### Example 53

Selectivity in Inhibition of HTT mRNA Levels Targeting SNP by Chimeric Oligonucleotides Designed by Microwalk

A series of modified oligonucleotides were designed based on the parent gapmer, ISIS 460209, wherein the central gap region comprises nine 2′-deoxyribonucleosides. These gapmers were created with various motifs and modifications at the wings and/or the central gap region.

The modified oligonucleotides and their motifs are described in Table 78. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e”, “k”, “y”, or “z” are sugar modified nucleosides. A subscript “e” indicates a 2′-O-methoxyethyl (MOE) modified nucleoside, a subscript “k” indicates a 6′-(S)—CH₃ bicyclic nucleoside (e.g. cEt), a subscript “y” indicates an α-L-LNA modified nucleoside, and a subscript “z” indicates a F-HNA modified nucleoside. ^{p}U indicates a 5-propyne uridine nucleoside and ^{x}T indicates a 2-thio-thymidine nucleoside. Underlined nucleosides indicate the mismatch position.

These gapmers were evaluated for thermal stability (Tₘ) using methods described in Example 42. Presented in Table 79 are the Tₘ measurements for chimeric antisense oligonucleotides when duplexed to mutant or wild-type RNA complement. The Tₘ of chimeric antisense oligonucleotides duplexed with mutant RNA complement is denoted as “Tₘ (° C.) mut”. The Tₘ of chimeric antisense oligonucleotides duplexed with wild-type RNA complement is denoted as “Tₘ (° C.) wt”.

These gapmers were also tested in vitro. ISIS 141923 was included in the study as a negative control and is denoted as “neg control”. The non-allele specific antisense oligonucleotides, ISIS 387916 was used as a positive control and is denoted as “pos control”. Heterozygous fibroblast GM04022 cell line was used. Cultured GM04022 cells at a density of 25,000 cells per well were transfected using electroporation with a single dose at 2 μM concentration of the modified oligonucleotide. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. HTT mRNA levels were adjusted according to total RNA content, as measured by RIBOGREEN. ISIS 460209 marked with an asterisk (*) in the table was included in the study for comparison. The results in Table 79 are presented as percent of HTT mRNA expression, relative to untreated control levels and is denoted as “% UTC”. Selectivity was also evaluated and measured by dividing the percent of wild-type HTT mRNA levels vs. the percent of mutant HTT mRNA levels.

As illustrated, several of the newly designed antisense oligonucleotides showed improvement in potency and/or selectivity in inhibiting mut HTT mRNA levels comparing to ISIS 460209.

TABLE 78
Modified oligonucleotides comprising various modifications targeting
HTT SNP

| | | | Wing | | SEQ |
|---|---|---|---|---|---|
| ISIS | | | Chemistry | | ID |
| NO | Sequence (5′ to 3′) | Modification | 5′ | 3′ | NO. |
| 460209* | TₑAₖAₖATTGTCATCAₖCₖCₑ | 3-9-3 | ekk | kke | 10 |
| | | (ekk-d9-kke) | | | |
| 539560 | TₑAₖAₖATTG^{p}UCATCAₖCₖCₑ | 5-propyne in | ekk | kke | 11 |
| | | gap | | | |
| 539563 | TₑAₖAₖATTG^{x}TCATCAₖCₖCₑ | 2-thio in gap | ekk | kke | 10 |
| 539554 | TₑAₖAₖATTGU_{y}CATCAₖCₖCₑ | α-L-LNA in gap | ekk | kke | 11 |
| 542686 | TₑAₖAₖATTGT_{z}CATCAₖCₖCₑ | F-HNA in gap | ekk | kke | 10 |
| 540108 | AₑTₑAₑAₖAₖTTGTCATCₖAₖCₑCₑAₑ | 5-7-5 | eeekk | kkeee | 23 |
| | | (eeekk-d7-kkeee) | | | |
| 544840 | TₑAₖAₖATTGTCATCAₖCₖCₑTₖTₖAₖ | 3-9-6 | ekk | kkekkk | 15 |
| | | (ekk-d9-kkekkk) | | | |
| 550904 | TₑAₖAₖATTGTCATCAₖCₖCₑTₖTₖTₖAₖ | 3-9-7 | ekk | kkekkkk | 18 |
| | | (ekk-d9-kkekkkk) | | | |
| 540082 | AₑTₖTₖGₖTCATCACCAGₖAₑ | 4-9-2 | ekkk | ke | 65 |
| | | (ekkk-d9-ke) | | | |
| 540089 | TₑTₖAₖAₖTAAATTGTCAₖTₑ | 4-9-2 | ekkk | ke | 66 |
| | | (ekkk-d9-ke) | | | |
| 540095 | AₑTₖTGTCATCACCₖAₖGₖAₑ | 2-9-4 | ek | kkke | 67 |
| | | (ek-d9-kkke) | | | |
| 543528 | AₑTₖAₑAₖAATGTCATCAₖCₑCₖAₑ | Mismatch at | ekek | keke | 77 |
| | | position 2 | | | |
| | | counting | | | |
| | | from 5′ gap | | | |
| 543533 | TₑAₖAₖATAGTCATCAₖCₖCₑ | Mismatch at | ekk | kke | 78 |
| | | position 3 | | | |
| | | counting | | | |
| | | from 5′ gap | | | |
| 387916 | TₑCₑTₑCₑTₑATTGCACATTCₑCₑAₑAₑGₑ | 5-10-5 | eeeee | eeeee | 56 |
| (pos control) | | | | | |
| 141923 | CₑCₑTₑTₑCₑCCTGAAGGTTCₑCₑTₑCₑCₑ | 5-10-5 | eeeee | eeeee | 57 |
| (neg control) | | | | | |

e = 2′-MOE; k = cEt; d = 2′-deoxyribonucleoside

TABLE 79
Comparison of selectivity in inhibition of HTT mRNA levels, and Tm of modified
oligonucleotides with ISIS 460209 targeted tors7685686 in GM04022 cells

| | Tm (° C.) | | % UTC | | Selectivity | | Wing Chemistry | |
|---|---|---|---|---|---|---|---|---|
| ISIS NO | mutant | wt | mut | wt | (wt vs mut) | Modification | 5′ | 3′ |
| 460209* | 53.7 | 52.2 | 23 | 57 | 2.7 | 3-9-3 | ekk | kke |
| | | | | | | (ekk-d9-kke) | | |
| 539560 | 54.1 | 50.8 | 13 | 32 | 2.4 | 5-propyne in gap | ekk | kke |
| 539563 | 53.8 | 49.1 | 13 | 40 | 3.2 | 2-thio in gap | ekk | kke |
| 539554 | 56.5 | 54.5 | 54 | 89 | 1.7 | α-L-LNA in gap | ekk | kke |
| 542686 | 56.1 | 50.4 | 26 | 62 | 2.4 | F-HNA in gap | ekk | kke |
| 540108 | 60.0 | 57.9 | 27 | 63 | 2.3 | 5-7-5 | eeekk | kkeee |
| | | | | | | (eeekk-d7-kkeee) | | |
| 544840 | — | — | 19 | 40 | 2.1 | 3-9-6 | ekk | kkekkk |
| | | | | | | (ekk-d9-kkekkk) | | |
| 550904 | — | — | 39 | 65 | 1.7 | 3-9-7 | ekk | kkekkkk |
| | | | | | | (ekk-d9- | | |
| | | | | | | kkekkkk) | | |
| 540082 | — | — | 21 | 62 | 3.0 | 4-9-2 | ekkk | ke |
| | | | | | | (ekkk-d9-ke) | | |
| 540089 | — | — | 78 | 86 | 1.1 | 4-9-2 | ekkk | ke |
| | | | | | | (ekkk-d9-ke) | | |
| 540095 | — | — | 22 | 66 | 3.1 | 2-9-4 | ek | kkke |
| | | | | | | (ek-d9-kkke) | | |
| 543528 | 50.5 | 49.1 | 44 | 90 | 2.1 | Mismatch at | ekek | keke |
| | | | | | | position 2 | | |
| | | | | | | counting from 5′ | | |
| | | | | | | gap | | |
| 543533 | 47.0 | 44.8 | 83 | 97 | 1.2 | Mismatch at | ekk | kke |
| | | | | | | position 3 | | |
| | | | | | | counting from 5′ | | |
| | | | | | | gap | | |
| 387916 | — | — | 21 | 19 | 0.9 | 5-10-5 | eeeee | eeeee |
| (pos control) | | | | | | | | |
| 141923 | — | — | 95 | 99 | 1.0 | 5-10-5 | eeeee | eeeee |
| (neg control) | | | | | | | | |

e = 2′-MOE;
k = cEt;
d = 2′-deoxyribonucleoside

### Example 54

### Chimeric Oligonucleotides Comprising Modifications at the SNP Site of HTT Gene

Additional gapmers are designed based on the gapmer selected from studies described in Tables 73 and 74 (ISIS 540108) and is marked with an asterisk (*). These gapmers are designed by introducing modifications at the SNP site at position 9 of the oligonucleotides, as counted from the 5′-terminus and are created with a 5-7-5 motif.

The gapmers are described in Table 80. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “a”, “b”, “e”, or “k” are sugar modified nucleosides. A subscript “a” indicates 2′-(ara)-F modified nucleoside, a subscript “b” indicates a 5′-Me DNA modified nucleoside, a subscript “e” indicates a 2′-O-methoxyethyl (MOE) modified nucleoside, and a subscript “k” indicates a 6′-(S)—CH₃ bicyclic nucleoside (e.g. cEt). ^{x}T indicates a 2-thio-thymidine nucleoside. Underline nucleoside or the number in parentheses indicates the position on the oligonucleotides opposite to the SNP position, as counted from the 5′-terminus.

TABLE 80
Modified oligonucleotides targeting HTT SNP

| | | | Wing | | SEQ |
|---|---|---|---|---|---|
| ISIS | | Gap | chemistry | | ID |
| NO | Sequence (5′ to 3′) | Chemistry | 5′ | 3′ | NO. |
| 540108* (9) | AₑTₑAₑAₖAₖTTGTCATCₖAₖCₑCₑAₑ | Deoxy | eeekk | kkeee | 32 |
| XXXX28 (9) | AₑTₑAₑAₖAₖTTG^{x}TCATCₖAₖCₑCₑAₑ | Deoxy/2- | eeekk | kkeee | 32 |
| | | thio | | | |
| XXXX29 (9) | AₑTₑAₑAₖAₖTTGTₐCATCₖAₖCₑCₑAₑ | Deoxy/2′- | eeekk | kkeee | 32 |
| | | (ara)-F | | | |
| XXXX30 (9) | AₑTₑAₑAₖAₖTTGT_{b}CATCₖAₖCₑCₑAₑ | Deoxy/5′- | eeekk | kkeee | 32 |
| | | Me-DNA | | | |

e = 2′-MOE, k = cEt

### Example 55

### Chimeric Oligonucleotides Comprising Modifications at the Wing Regions Targeting HTT SNP

Additional gapmers are designed based on the gapmer selected from studies described in Tables 89 and 21 (ISIS 540107) and is marked with an asterisk (*). These gapmers are designed by introducing bicyclic modified nucleosides at the 3′ or 5′ terminus and are tested to evaluate if the addition of bicyclic modified nucleosides at the wing regions improves the activity and selectivity in inhibition of mutant HTT SNP.

The gapmers comprise a 5-7-5 motif and are described in Table 81. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e”, or “k” are sugar modified nucleosides. A subscript “e” indicates a 2′-O-methoxyethyl (MOE) modified nucleoside, and a subscript “k” indicates a 6′-(S)—CH₃ bicyclic nucleoside (e.g. cEt).

TABLE 81
Modified oligonucleotides targeting HTT SNP

| | | | wing | | SEQ |
|---|---|---|---|---|---|
| ISIS | | | chemistry | | ID |
| NO | Sequence (5′ to 3′) | Motif | 5′ | 3′ | NO. |
| 540107* | AₑTₑAₑAₑAₖTTGTCATCₖAₑCₑCₑAₑ | 5-7-5 | eeeek | keeee | 32 |
| | | (eeeek-d7-keeee) | | | |
| XXXX31 | AₑTₑAₖAₖAₖTTGTCATCₖAₖCₖCₑAₑ | 5-7-5 | eekkk | kkkee | 32 |
| | | (eekkk-d7-kkkee) | | | |
| XXXX32 | AₑTₑAₑAₑAₖTTGTCATCₑAₑCₑCₑAₑ | 5-7-5 | eeeek | eeeee | 32 |
| | | (eeeek-d7-eeeee) | | | |
| XXXX33 | AₑTₑAₑAₖAₖTTGTCATCₑAₑCₑCₑAₑ | 5-7-5 | eeekk | eeeee | 32 |
| | | (eeekk-d7-eeeee) | | | |
| XXXX34 | AₑTₑAₖAₖAₖTTGTCATCₑAₑCₑCₑAₑ | 5-7-5 | eekkk | eeeee | 32 |
| | | (eekkk-d7-eeeee) | | | |
| XXXX35 | AₑTₑAₑAₑAₑTTGTCATCₖAₑCₑCₑAₑ | 5-7-5 | eeeee | keeee | 32 |
| | | (eeeee-d7-keeee) | | | |
| XXXX36 | AₑTₑAₑAₑAₑTTGTCATCₖAₖCₑCₑAₑ | 5-7-5 | eeeee | kkeee | 32 |
| | | (eeeee-d7-kkeee) | | | |
| XXXX37 | AₑTₑAₑAₑAₑTTGTCATCₖAₖCₖCₑAₑ | 5-7-5 | eeeee | kkkee | 32 |
| | | (eeeee-d7-kkkee) | | | |

e = 2′-MOE; k = cEt; d = 2′-deoxyribonucleoside

### Example 56

### Chimeric Oligonucleotides Comprising Wing and Central Gap Modifications Targeting HTT SNP

Additional gapmers are designed based on the parent gapmer, ISIS 460209, wherein the central gap region comprises nine 2′-deoxyribonucleosides and is marked with an asterisk (*) in the table. These gapmers were designed by introducing modifications at the wings or the central gap region and are created with a 3-9-3 motif.

The gapmers are described in Table 82. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e” or “k” are sugar modified nucleosides. A subscript “e” indicates a 2′-O-methoxyethyl (MOE) modified nucleoside, and a subscript “k” indicates a 6′-(S)—CH₃ bicyclic nucleoside (e.g. cEt). ^{P}T indicates a 5-propyne thymidine nucleoside. ^{P}C indicates a 5-propyne cytosine nucleoside. Underline nucleoside or the number in parentheses indicates the position on the oligonucleotides opposite to the SNP position, as counted from the 5′-terminus.

TABLE 82
Modified oligonucleotides targeting HTT SNP

| | | | wing | | SEQ |
|---|---|---|---|---|---|
| ISIS | | | chemistry | | ID |
| NO | Sequence (5′ to 3′) | Modification | 5′ | 3′ | NO |
| 460209* (8) | TₑAₖAₖATTGTCATCAₖCₖCₑ | Deoxy gap | ekk | kke | 10 |
| | | (3-9-3) | | | |
| 552103 (8) | TₑAₑAₑATTGTCATCAₖCₖCₖ | Deoxy gap | eee | kkk | 10 |
| | | (3-9-3) | | | |
| 552104 (8) | TₖAₖAₖATTGTCATCAₑCₑCₑ | Deoxy gap | kkk | eee | 10 |
| | | (3-9-3) | | | |
| 552105 (8) | TₑAₖAₖATTG^{P}T^{P}CATCAₖCₖCₑ | Deoxy/5- | ekk | kke | 10 |
| | | Propyne | | | |
| 552106 (8) | TₑAₖAₖA^{P}T^{P}TG^{P}T^{P}CA^{P}T^{P}CAₖCₖCₑ | Deoxy/5- | ekk | kke | 10 |
| | | Propyne | | | |

e = 2′-MOE; k = cEt

### Example 57

### Modified Oligonucleotides Comprising F-HNA Modification at the Central Gap or Wing Region Targeting HTT SNP

A series of modified oligonucleotides were designed based on ISIS 460209, wherein the central gap region contains nine 2′-deoxyribonucleosides. These modified oligonucleotides were designed by incorporating one or more F-HNA(s) modification within the central gap region or on the wing regions. The F-HNA containing oligonucleotides were tested for their ability to selectively inhibit mutant (mut) HTT mRNA expression levels targeting rs7685686 while leaving the expression of the wild-type (wt) intact. The activity and selectivity of the modified oligonucleotides were evaluated and compared to ISIS 460209.

The modified oligonucleotides and their motifs are described in Table 83. The internucleoside linkages throughout each modified oligonucleotide are phosphorothioate linkages (P═S). Nucleosides without a subscript are β-D-2′-deoxyribonucleosides. Nucleosides followed by a subscript “e” indicate 2′-O-methoxyethyl (MOE) modified nucleosides. Nucleosides followed by a subscript “k” indicate 6′-(S)—CH₃ bicyclic nucleosides (e.g. cEt). Nucleosides followed by a subscript “z” indicate F-HNA modified nucleosides. ^{m}C indicates a 5-methyl cytosine nucleoside. Underlined nucleoside indicates the position on the oligonucleotides opposite to the SNP position, which is position 8 as counted from the 5′-terminus.

The gap-interrupted antisense oligonucleotides were tested in vitro. Heterozygous fibroblast GM04022 cell line was used. Cultured GM04022 cells at a density of 25,000 cells per well were transfected using electroporation with 0.12, 0.37, 1.1, 3.3 and 10 μM concentrations of modified oligonucleotides. After a treatment period of approximately 16 hours, RNA was isolated from the cells and mRNA levels were measured by quantitative real-time PCR using ABI assay C_2229297_10 which measures at dbSNP rs362303. The HTT mRNA levels were adjusted according to total RNA content, as measured by RIBOGREEN and the results are presented in Table 84.

The IC₅₀ and selectivity were calculated using methods previously described in Example 41. The IC₅₀ at which each oligonucleotide inhibits the mutant HTT mRNA expression is denoted as ‘mut IC₅₀’. The IC₅₀ at which each oligonucleotide inhibits the wild-type HTT mRNA expression is denoted as ‘wt IC₅₀’. Selectivity was calculated by dividing the IC₅₀ for inhibition of the wild-type HTT versus the IC₅₀ for inhibiting expression of the mutant HTT mRNA.

The parent gapmer, 460209 is marked with an asterisk (*) in the table and was included in the study as a benchmark oligonucleotide against which the activity and selectivity of antisense oligonucleotides targeting nucleotides overlapping the SNP position could be compared.

As illustrated in Table 84, oligonucleotides comprising F-HNA modification(s) showed improvement in selectivity while maintaining activity as compared to the parent gapmer, ISIS 460209.

TABLE 83
Gap-interrupted antisense oligonucleotides targeting
HTT SNP

| | | | | Wing | | SEQ |
|---|---|---|---|---|---|---|
| ISIS | Sequence | | Gap | chemistry | | ID |
| NO. | (5′ to 3′) | Motif | chemistry | 5′ | 3′ | NO. |
| 460209* | TₑAₖA^{k}ATTGT | 3-9-3 | Full deoxy | ekk | kke | 10 |
| | ^{m}CAT^{m}CAₖ^{m}Cₖ^{m}Cₑ | | | | | |
| 566266 | TₑAₖAₖA_{z}TTGT | 3-9-3 or | Deoxy/F- | ekk or | kke | 10 |
| | ^{m}CAT^{m}CAₖ^{m}Cₖ^{m}Cₑ | 4-8-3 | HNA | ekkz | | |
| 566267 | TₑAₖAₖAT_{z}TGT | 3-9-3 or | Deoxy/F- | ekk or | kke | 10 |
| | ^{m}CAT^{m}CAₖ^{m}Cₖ^{m}Cₑ | 5-7-3 | HNA | ekkdz | | |
| 566268 | TₑAₖAₖATT_{z}GT | 3-9-3 or | Deoxy/F- | ekk or | kke | 10 |
| | ^{m}CAT^{m}CAₖ^{m}Cₖ^{m}Cₑ | 6-6-3 | HNA | ekkddz | | |
| 566269 | TₑAₖAₖATTG_{z}T | 3-9-3 or | Deoxy/F- | ekk or | kke | 10 |
| | ^{m}CAT^{m}CAₖ^{m}Cₖ^{m}Cₑ | 7-5-3 | HNA | ekkdddz | | |
| 567369 | TₑAₖAₖA_{z}T_{z}TGT | 3-9-3 or | Deoxy/F- | ekk or | kke | 10 |
| | ^{m}CAT^{m}CAₖ^{m}Cₖ^{m}Cₑ | 5-7-3 | HNA | ekkzz | | |

e = 2′-MOE, k = cEt, d = 2′-β-deoxyribonucleoside, z = F-HNA

TABLE 84
Comparison of inhibition of HTT mRNA levels and selectivity
of gap-interrupted antisense oligonucleotides with
ISIS 460209 targeting HTT SNP

| | IC₅₀ (μM) | | Selectivity | | Gap | Wing Chemistry | |
|---|---|---|---|---|---|---|---|
| ISIS NO | Mut | Wt | (wt vs mut) | Motif | chemistry | 5′ | 3′ |
| 460209* | 0.28 | 3.1 | 11 | 3-9-3 | Full deoxy | ekk | kke |
| 566266 | 0.20 | >10 | >50 | 3-9-3 or | Deoxy/F- | ekk or ekkz | kke |
| | | | | 4-8-3 | HNA | | |
| 566267 | 0.90 | >9.9 | >11 | 3-9-3 or | Deoxy/F- | ekk or ekkdz | kke |
| | | | | 5-7-3 | HNA | | |
| 566268 | 1.0 | >10 | >10 | 3-9-3 or | Deoxy/F- | ekk or ekkddz | kke |
| | | | | 6-6-3 | HNA | | |
| 566269 | 1.7 | >10.2 | >6 | 3-9-3 or | Deoxy/F- | ekk or | kke |
| | | | | 7-5-3 | HNA | ekkdddz | |
| 567369 | 0.82 | >9.8 | >12 | 3-9-3 or | Deoxy/F- | ekk or ekkzz | kke |
| | | | | 5-7-3 | HNA | | |

e = 2′-MOE,
k = cEt,
d = 2′-β-deoxyribonucleoside,
z = F-HNA

### Example 58

### Modified Oligonucleotides Comprising cEt Modification(s) at the Central Gap Region Targeting HTT SNP

A series of modified oligonucleotides were designed in the same manner as described in Example 57. These modified oligonucleotides were designed by replacing F-HNA(s) with cEt modification(s) in the central gap region while maintaining the wing configuration. The modified oligonucleotides were tested for their ability to selectively inhibit mutant (mut) HTT mRNA expression levels targeting rs7685686 while leaving the expression of the wild-type (wt) intact. The activity and selectivity of the modified oligonucleotides were evaluated and compared to ISIS 460209.

The modified oligonucleotides and their motifs are described in Table 85. The internucleoside linkages throughout each modified oligonucleotide are phosphorothioate linkages (P═S). Nucleosides without a subscript are β-D-2′-deoxyribonucleosides. Nucleosides followed by a subscript “e” indicate 2′-O-methoxyethyl (MOE) modified nucleosides. Nucleosides followed by a subscript “k” indicate 6′-(S)—CH₃ bicyclic nucleosides (e.g. cEt). ^{m}C indicates a 5-methyl cytosine nucleoside. Underlined nucleoside indicates the position on the oligonucleotides opposite to the SNP position, which is position 8 as counted from the 5′-terminus.

The gap-interrupted antisense oligonucleotides were tested in vitro. Heterozygous fibroblast GM04022 cell line was used. Cultured GM04022 cells at a density of 25,000 cells per well were transfected using electroporation with 0.12, 0.37, 1.1, 3.3 and 10 μM concentrations of modified oligonucleotides. After a treatment period of approximately 16 hours, RNA was isolated from the cells and mRNA levels were measured by quantitative real-time PCR using ABI assay C_2229297_10 which measures at dbSNP rs362303. The HTT mRNA levels were adjusted according to total RNA content, as measured by RIBOGREEN and the results are presented below.

The IC₅₀ and selectivity were calculated using methods previously described in Example 41. The IC₅₀ at which each oligonucleotide inhibits the mutant HTT mRNA expression is denoted as ‘mut IC₅₀’. The IC₅₀ at which each oligonucleotide inhibits the wild-type HTT mRNA expression is denoted as ‘wt IC₅₀’. Selectivity was calculated by dividing the IC₅₀ for inhibition of the wild-type HTT versus the IC₅₀ for inhibiting expression of the mutant HTT mRNA.

As illustrated in Table 86, some of the newly designed antisense oligonucleotides (ISIS 575006, 575007, and 575008) showed improvement in potency and/or selectivity in inhibiting mut HTT mRNA levels comparing to ISIS 460209.

TABLE 85
Gap-interrupted antisense oligonucleotides targeting
HTT SNP

| | | | | Wing | | SEQ |
|---|---|---|---|---|---|---|
| ISIS | Sequence | | Gap | chemistry | | ID |
| NO. | (5′ to 3′) | Motif | chemistry | 5′ | 3′ | NO. |
| 460209* | TₑAₖAₖATTGT | 3-9-3 | Full deoxy | ekk | kke | 10 |
| | ^{m}CAT^{m}CAₖ^{m}Cₖ^{m}Cₑ | | | | | |
| 575006 | TₑAₖAₖAₖTTGT | 4-8-3 | Full deoxy | ekkk | kke | 10 |
| | ^{m}CAT^{m}CAₖ^{m}Cₖ^{m}Cₑ | | | | | |
| 575007 | TₑAₖAₖATₖTGT | 3-9-3 or | Full deoxy or | ekk or | kke | 10 |
| | ^{m}CAT^{m}CAₖ^{m}Cₖ^{m}Cₑ | 5-7-3 | Deoxy/cEt | ekkdk | | |
| 575133 | TₑAₖAₖATTₖGT | 3-9-3 or | Full deoxy or | ekk or | kke | 10 |
| | ^{m}CAT^{m}CAₖ^{m}Cₖ^{m}Cₑ | 6-6-3 | Deoxy/cEt | ekkddk | | |
| 575134 | TₑAₖAₖATTGₖT | 3-9-3 or | Full deoxy or | ekk or | kke | 10 |
| | ^{m}CAT^{m}CAₖ^{m}Cₖ^{m}Cₑ | 7-5-3 | Deoxy/cEt | ekkdddk | | |
| 575008 | TₑAₖAₖAₖTₖTGT | 5-7-3 | Deoxy | ekkkk | kke | 10 |
| | ^{m}CAT^{m}CAₖ^{m}Cₖ^{m}Cₑ | | | | | |

e = 2′-MOE, k = cEt, d = 2′-β-deoxyribonucleoside

TABLE 86
Comparison of inhibition of HTT mRNA levels and selectivity of gap-interrupted
antisense oligonucleotides with ISIS 460209 targeting HTT SNP

| | IC₅₀ (μM) | | Selectivity | | Gap | Wing Chemistry | |
|---|---|---|---|---|---|---|---|
| ISIS NO | Mut | Wt | (wt vs mut) | Motif | chemistry | 5′ | 3′ |
| 460209* | 0.28 | 3.1 | 11 | 3-9-3 | Full deoxy | ekk | kke |
| 575006 | 0.27 | 3.8 | 14 | 4-8-3 | Full deoxy | ekkk | kke |
| 575007 | 0.67 | >10.1 | >15 | 3-9-3 or | Full deoxy or | ekk or | kke |
| | | | | 5-7-3 | Deoxy/cEt | ekkdk | |
| 575133 | 3.0 | >9 | >3 | 3-9-3 or | Full deoxy or | ekk or | kke |
| | | | | 6-6-3 | Deoxy/cEt | ekkddk | |
| 575134 | 2.6 | >10.4 | >4 | 3-9-3 or | Full deoxy or | ekk or | kke |
| | | | | 7-5-3 | Deoxy/cEt | ekkdddk | |
| 575008 | 0.18 | >9.9 | >55 | 5-7-3 | Full deoxy | ekkkk | kke |

e = 2′-MOE,
k = cEt,
d = 2′-β-deoxyribonucleoside

### Example 59

### Modified Oligonucleotides Comprising F-HNA Modification at the 3′-End of Central Gap Region Targeting HTT SNP

A series of modified oligonucleotides were designed based on ISIS 460209, wherein the central gap region contains nine 2′-deoxyribonucleosides. These modified oligonucleotides were designed by incorporating one F-HNA modification at the 3′-end of the central gap region. The F-HNA containing oligonucleotides were tested for their ability to selectively inhibit mutant (mut) HTT mRNA expression levels targeting HTT SNP while leaving the expression of the wild-type (wt) intact. The activity and selectivity of the modified oligonucleotides were evaluated and compared to ISIS 460209.

The modified oligonucleotides and their motifs are described in Table 87. The internucleoside linkages throughout each modified oligonucleotide are phosphorothioate linkages (P═S). Nucleosides without a subscript are β-D-2′-deoxyribonucleosides. Nucleosides followed by a subscript “e” indicate 2′-O-methoxyethyl (MOE) modified nucleosides. Nucleosides followed by a subscript “k” indicate 6′-(S)—CH₃ bicyclic nucleosides (e.g. cEt). Nucleosides followed by a subscript “z” indicate F-HNA modified nucleosides. ^{m}C indicates a 5-methyl cytosine nucleoside. Underlined nucleoside indicates the position on the oligonucleotides opposite to the SNP position, which is position 8 as counted from the 5′-terminus.

The modified oligonucleotides were tested in vitro. Heterozygous fibroblast GM04022 cell line was used. Cultured GM04022 cells at a density of 25,000 cells per well were transfected using electroporation with 0.12, 0.37, 1.1, 3.3 and 10 μM concentrations of modified oligonucleotides. After a treatment period of approximately 16 hours, RNA was isolated from the cells and mRNA levels were measured by quantitative real-time PCR using ABI assay C_2229297_10 which measures at dbSNP rs362303. The HTT mRNA levels were adjusted according to total RNA content, as measured by RIBOGREEN and the results are presented in Table 88.

The IC₅₀ and selectivity were calculated using methods previously described in Example 41. The IC₅₀ at which each oligonucleotide inhibits the mutant HTT mRNA expression is denoted as ‘mut IC₅₀’. The IC₅₀ at which each oligonucleotide inhibits the wild-type HTT mRNA expression is denoted as ‘wt IC₅₀’. Selectivity was calculated by dividing the IC₅₀ for inhibition of the wild-type HTT versus the IC₅₀ for inhibiting expression of the mutant HTT mRNA.

As illustrated in Table 88, a couple of the newly designed antisense oligonucleotides (ISIS 575833 and 575834) showed improvement in selectivity while maintaining potency as compared to ISIS 460209. ISIS 575836 showed an increase in potency without improvement in selectivity while ISIS 575835 showed comparable selectivity without improvement in potency.

TABLE 87
Modified oligonucleotides targeting HTT SNP

| | | | Gap | Wing | | SEQ |
|---|---|---|---|---|---|---|
| ISIS | Sequence | | chem- | chemistry | | ID |
| NO. | (5′ to 3′) | Motif | istry | 5′ | 3′ | NO. |
| 460209* | TₑAₖAₖATTGT | 3-9-3 | Full | ekk | kke | 10 |
| | ^{m}CAT^{m}CAₖ^{m}Cₖ^{m}Cₑ | | deoxy | | | |
| 575833 | TₑAₖAₖATTGT | 3-9-3 | Deoxy/ | ekk | kke or | 10 |
| | ^{m}C_{z}AT^{m}CAₖ^{m}Cₖ^{m}Cₑ | or | F-HNA | | zdddkke | |
| | | 3-5-7 | | | | |
| 575834 | TₑAₖAₖATTGT | 3-9-3 | Deoxy/ | ekk | kke or | 10 |
| | ^{m}CA_{z}T^{m}CAₖ^{m}Cₖ^{m}Cₑ | or | F-HNA | | zddkke | |
| | | 3-6-6 | | | | |
| 575835 | TₑAₖAₖATTGT | 3-9-3 | Deoxy/ | ekk | kke or | 10 |
| | ^{m}CAT_{z}^{m}CAₖ^{m}Cₖ^{m}Cₑ | or | F-HNA | | zdkke | |
| | | 3-7-5 | | | | |
| 575836 | TₑAₖAₖATTGT | 3-9-3 | Deoxy/ | ekk | kke or | 10 |
| | ^{m}CAT^{m}C_{z}Aₖ^{m}Cₖ^{m}Cₑ | or | F-HNA | | zkke | |
| | | 3-8-4 | | | | |

e = 2′-MOE, k = cEt, d = 2′-β-deoxyribonucleoside, z = F-HNA

TABLE 88
Comparison of inhibition of HTT mRNA levels and selectivity
of modified oligonucleotides with ISIS 460209 targeting HTT SNP

| | IC₅₀ (μM) | | Selectivity | | | Wing Chemistry | |
|---|---|---|---|---|---|---|---|
| ISIS NO | Mut | Wt | (wt vs mut) | Motif | Gap chemistry | 5′ | 3′ |
| 460209* | 0.28 | 3.1 | 11 | 3-9-3 | Full deoxy | ekk | kke |
| 575833 | 0.22 | 4.2 | 19 | 3-9-3 or | Deoxy/F-HNA | ekk | kke or |
| | | | | 3-5-7 | | | zdddkke |
| 575834 | 0.30 | 6.3 | 21 | 3-9-3 or | Deoxy/F-HNA | ekk | kke or |
| | | | | 3-6-6 | | | zddkke |
| 575835 | 0.89 | 9.8 | 11 | 3-9-3 or | Deoxy/F-HNA | ekk | kke or |
| | | | | 3-7-5 | | | zdkke |
| 575836 | 0.09 | 0.4 | 4.6 | 3-9-3 or | Deoxy/F-HNA | ekk | kke or zkke |
| | | | | 3-8-4 | | | |

e = 2′-MOE,
k = cEt,
d = 2′-β-deoxyribonucleoside,
z = F-HNA

### Example 60

### Short-Gap Chimeric Oligonucleotides Targeting Huntingtin (HTT) Single Nucleotide Polymorphism (SNP)

Additional chimeric antisense oligonucleotides were designed based on ISIS 460209 and ISIS 540094 wherein the central gap region contains nine 2′-deoxynucleosides. These gapmers were designed with the central gap region shortened by introducing cEt modifications to the wing regions, or interrupted by introducing cEt modifications at the 3′-end of the central gap region. The modified oligonucleotides were tested for their ability to selectively inhibit mutant (mut) HTT mRNA expression levels targeting HTT SNP while leaving the expression of the wild-type (wt) intact. The activity and selectivity of the modified oligonucleotides were evaluated and compared to ISIS 460209 and 540094.

The gapmers and their motifs are described in Table 89. The internucleoside linkages throughout each modified oligonucleotide are phosphorothioate linkages (P═S). Nucleosides without a subscript are (3-D-2′-deoxyribonucleosides. Nucleosides followed by a subscript “e” indicate 2′-O-methoxyethyl (MOE) modified nucleosides. Nucleosides followed by a subscript “k” indicate 6′-(S)—CH₃ bicyclic nucleosides (e.g. cEt). ^{m}C indicates a 5-methyl cytosine nucleoside. Underlined nucleoside indicates the position on the oligonucleotides opposite to the SNP position, which is position 4 or 8 as counted from the 5′-terminus.

The modified oligonucleotides were tested in vitro. Heterozygous fibroblast GM04022 cell line was used. Cultured GM04022 cells at a density of 25,000 cells per well were transfected using electroporation with 0.12, 0.37, 1.1, 3.3 and 10 μM concentrations of modified oligonucleotides. After a treatment period of approximately 16 hours, RNA was isolated from the cells and mRNA levels were measured by quantitative real-time PCR using ABI assay C_2229297_10 which measures at dbSNP rs362303. The HTT mRNA levels were adjusted according to total RNA content, as measured by RIBOGREEN and the results are presented in Table 90.

The IC₅₀ and selectivity were calculated using methods previously described in Example 41. The IC₅₀ at which each oligonucleotide inhibits the mutant HTT mRNA expression is denoted as ‘mut IC₅₀’. The IC₅₀ at which each oligonucleotide inhibits the wild-type HTT mRNA expression is denoted as ‘wt IC₅₀’. Selectivity was calculated by dividing the IC₅₀ for inhibition of the wild-type HTT versus the IC₅₀ for inhibiting expression of the mutant HTT mRNA.

As illustrated in Table 90, the newly designed antisense oligonucleotides (ISIS 575003) showed improvement in selectivity while maintaining potency as compared to ISIS 460209.

TABLE 89
Short-gap antisense oligonucleotides
targeting HTT SNP

| | | | | Wing | | SEQ |
|---|---|---|---|---|---|---|
| ISIS | Sequence | | Gap | chemistry | | ID |
| NO. | (5′ to 3′) | Motif | chemistry | 5′ | 3′ | NO. |
| 460209* | TₑAₖAₖATTGT | 3-9-3 | Full | ekk | kke | 10 |
| | ^{m}CAT^{m}CAₖ^{m}Cₖ^{m}Cₑ | | deoxy | | | |
| 540094* | TₑTₖGT^{m}CAT^{m}CA | 2-9-4 | Full | ek | kkke | 67 |
| | ^{m}C^{m}CAₖGₖAₖAₑ | | deoxy | | | |
| 575003 | TₑTₖGT^{m}CAT^{m}CA | 2-8-5 | Full | ek | kkkke | 67 |
| | ^{m}C^{m}CₖAₖGₖAₖAₑ | | deoxy | | | |
| 575004 | TₑTₖGT^{m}CAT^{m}CA | 2-9-4 | Full | ek | kkke | 67 |
| | ^{m}Cₖ^{m}CAₖGₖAₖAₑ | or | deoxy | | or | |
| | | 2-7-6 | or | | kdkkke | |
| | | | Deoxy/cEt | | | |
| 575005 | TₑTₖGT^{m}CAT^{m}CA | 2-7-6 | Full | ek | kkkkke | 67 |
| | ^{m}Cₖ^{m}CₖAₖGₖAₖAₑ | | deoxy | | | |

e = 2′-MOE, k = cEt, d = 2′-deoxyribonucleoside

TABLE 90
Comparison of inhibition of HTT mRNA levels and selectivity
of modified oligonucleotides with ISIS 460209 targeting HTT SNP

| | IC₅₀ (μM) | | Selectivity | | | Wing Chemistry | |
|---|---|---|---|---|---|---|---|
| ISIS NO | Mut | Wt | (wt vs mut) | Motif | Gap chemistry | 5′ | 3′ |
| 460209* | 0.34 | 3.3 | 9.7 | 3-9-3 | Full deoxy | ekk | kke |
| 540094* | 0.17 | 2.4 | 14 | 2-9-4 | Full deoxy | ek | kkke |
| 575003 | 0.40 | 10 | 25 | 2-8-5 | Full deoxy | ek | kkkke |
| 575004 | 1.2 | >9.6 | >8 | 2-9-4 or | Full deoxy or | ek | kkke or |
| | | | | 2-7-6 | Deoxy/cEt | | kdkkke |
| 575005 | >10 | >100 | >10 | 2-7-6 | Full deoxy | ek | kkkkke |

e = 2′-MOE,
k = cEt,
d = 2′-deoxyribonucleoside

### Example 61

### Short-Gap Chimeric Oligonucleotides Targeting Huntingtin (HTT) Single Nucleotide Polymorphism (SNP)

Additional chimeric antisense oligonucleotides were designed based on 15-mer, ISIS 460209 and 17-mer, ISIS 476333 wherein the central gap region contains nine 2′-deoxynucleosides. These gapmers were designed with the central gap region shortened at the 5′-end of the central gap region. The gapmers were tested for their ability to selectively inhibit mutant (mut) HTT mRNA expression levels targeting HTT SNP while leaving the expression of the wild-type (wt) intact. The activity and selectivity of the gapmers were evaluated and compared to ISIS 460209 and ISIS 476333.

The gapmers and their motifs are described in Table 91. The internucleoside linkages throughout each modified oligonucleotide are phosphorothioate linkages (P═S). Nucleosides without a subscript are (3-D-2′-deoxyribonucleosides. Nucleosides followed by a subscript “e” indicate 2′-O-methoxyethyl (MOE) modified nucleosides. Nucleosides followed by a subscript “k” indicate 6′-(S)—CH₃ bicyclic nucleosides (e.g. cEt). ^{m}C indicates a 5-methyl cytosine nucleoside. Underlined nucleoside indicates the position on the oligonucleotides opposite to the SNP position, which is position 8 or 9 as counted from the 5′-terminus.

The modified oligonucleotides were tested in vitro. Heterozygous fibroblast GM04022 cell line was used. Cultured GM04022 cells at a density of 25,000 cells per well were transfected using electroporation with 0.12, 0.37, 1.1, 3.3 and 10 μM concentrations of modified oligonucleotides. After a treatment period of approximately 16 hours, RNA was isolated from the cells and mRNA levels were measured by quantitative real-time PCR using ABI assay C_2229297_10 which measures at dbSNP rs362303. The HTT mRNA levels were adjusted according to total RNA content, as measured by RIBOGREEN and the results are presented in Table 92.

The IC₅₀ and selectivity were calculated using methods previously described in Example 41. The IC₅₀ at which each oligonucleotide inhibits the mutant HTT mRNA expression is denoted as ‘mut IC₅₀’. The IC₅₀ at which each oligonucleotide inhibits the wild-type HTT mRNA expression is denoted as ‘wt IC₅₀’. Selectivity was calculated by dividing the IC₅₀ for inhibition of the wild-type HTT versus the IC₅₀ for inhibiting expression of the mutant HTT mRNA.

As illustrated in Table 92, a couple of the newly designed antisense oligonucleotides (ISIS 571036 and 571037) showed improvement in potency and selectivity in inhibiting mut HTT mRNA levels as compared to ISIS 460209 and 476333.

TABLE 91
Short-gap antisense oligonucleotides
targeting HTT SNP

| | | | Gap | Wing | | SEQ |
|---|---|---|---|---|---|---|
| ISIS | Sequence | | chem- | chemistry | | ID |
| NO. | (5′ to 3′) | Motif | istry | 5′ | 3′ | NO. |
| 460209* | TₑAₖAₖATTGT | 3-9-3 | Full | ekk | kke | 10 |
| | ^{m}CAT^{m}CAₖ^{m}Cₖ^{m}Cₑ | | deoxy | | | |
| 476333* | AₑTₖAₑAₖATTGT | 4-9-4 | Full | ekek | keke | 32 |
| | ^{m}CAT^{m}CAₖ^{m}Cₑ^{m}CₖAₑ | | deoxy | | | |
| 571036 | AₑTₖAₑAₖAₑTₖTGT | 6-7-4 | Full | ekekek | keke | 32 |
| | ^{m}CAT^{m}CAₖ^{m}Cₑ^{m}CₖAₑ | | deoxy | | | |
| 571037 | AₑTₑAₑAₑAₖTₖTGT | 6-7-4 | Full | eeeekk | keke | 32 |
| | ^{m}CAT^{m}CAₖ^{m}Cₑ^{m}CₖAₑ | | deoxy | | | |
| 571038 | AₑTₖAₑAₖAₑTₑTGT | 6-7-4 | Full | ekekee | keke | 32 |
| | ^{m}CAT^{m}CAₖ^{m}Cₑ^{m}CₖAₑ | | deoxy | | | |

e = 2′-MOE, k = cEt, d = 2′-deoxyribonucleoside

TABLE 92
Comparison of inhibition of HTT mRNA levels and selectivity of
modified oligonucleotides with ISIS 460209 targeting HTT SNP

| | | | Selectivity | | | Wing | |
|---|---|---|---|---|---|---|---|
| | IC₅₀ (μM) | | (wt vs | | Gap | Chemistry | |
| ISIS NO | Mut | Wt | mut) | Motif | chemistry | 5′ | 3′ |
| 460209* | 0.34 | 3.3 | 9.7 | 3-9-3 | Full deoxy | ekk | kke |
| 476333* | 0.32 | 1.5 | 4.7 | 4-9-4 | Full deoxy | ekek | keke |
| 571036 | 0.17 | >10.0 | >59 | 6-7-4 | Full deoxy | ekekek | keke |
| 571037 | 0.11 | >9.9 | >90 | 6-7-4 | Full deoxy | eeeekk | keke |
| 571038 | 1.5 | >10.5 | >7 | 6-7-4 | Full deoxy | ekekee | keke |

e = 2′-MOE,
k = cEt,
d = 2′-deoxyribonucleoside

### Example 62

### Short-Gap Chimeric Oligonucleotides Targeting Huntingtin (HTT) Single Nucleotide Polymorphism (SNP)

Additional chimeric antisense oligonucleotides were designed based on 15-mer, ISIS 460209 wherein the central gap region contains nine 2′-deoxynucleosides. These gapmers were designed by having the central gap region shortened to seven 2′-deoxynucleosides. The gapmers were tested for their ability to selectively inhibit mutant (mut) HTT mRNA expression levels targeting HTT SNP while leaving the expression of the wild-type (wt) intact. The activity and selectivity of the gapmers were evaluated and compared to ISIS 460209.

The gapmers and their motifs are described in Table 93. The internucleoside linkages throughout each modified oligonucleotide are phosphorothioate linkages (P═S). Nucleosides without a subscript are (3-D-2′-deoxyribonucleosides. Nucleosides followed by a subscript “e” indicate 2′-O-methoxyethyl (MOE) modified nucleosides. Nucleosides followed by a subscript “k” indicate 6′-(S)—CH₃ bicyclic nucleosides (e.g. cEt). ^{m}C indicates a 5-methyl cytosine nucleoside. Underlined nucleoside indicates the position on the oligonucleotides opposite to the SNP position, which is position 8 or 9 as counted from the 5′-terminus.

The modified oligonucleotides were tested in vitro. Heterozygous fibroblast GM04022 cell line was used. Cultured GM04022 cells at a density of 25,000 cells per well were transfected using electroporation with 0.12, 0.37, 1.1, 3.3 and 10 μM concentrations of modified oligonucleotides. After a treatment period of approximately 16 hours, RNA was isolated from the cells and mRNA levels were measured by quantitative real-time PCR using ABI assay C_2229297_10 which measures at dbSNP rs362303. The HTT mRNA levels were adjusted according to total RNA content, as measured by RIBOGREEN and the results are presented in Table 94.

The IC₅₀ and selectivity were calculated using methods previously described in Example 41. The IC₅₀ at which each oligonucleotide inhibits the mutant HTT mRNA expression is denoted as ‘mut IC₅₀’. The IC₅₀ at which each oligonucleotide inhibits the wild-type HTT mRNA expression is denoted as ‘wt IC₅₀’. Selectivity was calculated by dividing the IC₅₀ for inhibition of the wild-type HTT versus the IC₅₀ for inhibiting expression of the mutant HTT mRNA.

As illustrated in Table 94, each of the newly designed antisense oligonucleotides (ISIS 540108 and 571069) showed improvement in potency and/or selectivity in inhibiting mut HTT mRNA levels as compared to ISIS 460209.

TABLE 93
Short-gap antisense oligonucleotides
targeting HTT SNP

| | | | Gap | Wing | | SEQ |
|---|---|---|---|---|---|---|
| ISIS | Sequence | Mo- | chem- | chemistry | | ID |
| NO. | (5′ to 3′) | tif | istry | 5′ | 3′ | NO. |
| 460209 | TₑAₖAₖATTGT | 3-9- | Full | ekk | kke | 10 |
| | ^{m}CAT^{m}CAₖ^{m}Cₖ^{m}Cₑ | 3 | deoxy | | | |
| 540108 | AₑTₑAₑAₖAₖTTGT | 5-7- | Full | eeekk | kkeee | 32 |
| | ^{m}CAT^{m}CₖAₖ^{m}Cₑ^{m}CₑAₑ | 5 | deoxy | | | |
| 571069 | AₑTₑAₑAₑAₖTₖTGT | 6-7- | Full | eeeekk | kkee | 32 |
| | ^{m}CAT^{m}CAₖ^{m}Cₖ^{m}CₑAₑ | 4 | deoxy | | | |
| 571173 | AₑTₑAₖAₖATTGT | 4-7- | Full | eekk | kkeeee | 32 |
| | ^{m}CATₖ^{m}CₖAₑ^{m}Cₑ^{m}CₑAₑ | 6 | deoxy | | | |
| 572773 | TₑAₑAₖAₖTTGT | 4-7- | Full | eekk | kkee | 10 |
| | ^{m}CAT^{m}CₖAₖ^{m}Cₑ^{m}Cₑ | 4 | deoxy | | | |

e = 2′-MOE, k = cEt, d = 2′-deoxyribonucleoside

TABLE 94
Comparison of inhibition of HTT mRNA levels and selectivity
of modified oligonucleotides with ISIS 460209 targeting HTT SNP

| | | | Selectivity | | | | |
|---|---|---|---|---|---|---|---|
| ISIS | IC₅₀ (μM) | | (wt vs | | Gap | Wing Chemistry | |
| NO | Mut | Wt | mut) | Motif | chemistry | 5′ | 3′ |
| 460209 | 0.34 | 3.3 | 9.7 | 3-9-3 | Full deoxy | ekk | kke |
| 540108 | 0.20 | >10 | >50 | 5-7-5 | Full deoxy | eeekk | kkeee |
| 571069 | 0.29 | >9.9 | >34 | 6-7-4 | Full deoxy | eeeekk | kkee |
| 571173 | 1.0 | >10 | >10 | 4-7-6 | Full deoxy | eekk | kkeeee |
| 572773 | 0.71 | >7.8 | 11 | 4-7-4 | Full deoxy | eekk | kkee |

e = 2′-MOE,
k = cEt,
d = 2′-deoxyribonucleoside

### Example 63

### Short-Gap Chimeric Oligonucleotides Targeting Huntingtin (HTT) Single Nucleotide Polymorphism (SNP)

Additional chimeric antisense oligonucleotides were designed based on 15-mer, ISIS 460209 and 17-mer, ISIS 540108 wherein the central gap region contains nine and seven 2′-deoxynucleosides, respectively. These gapmers were designed by introducing one or more cEt modification(s) at the 5′-end of the central gap region. The gapmers were tested for their ability to selectively inhibit mutant (mut) HTT mRNA expression levels targeting HTT SNP while leaving the expression of the wild-type (wt) intact. The activity and selectivity of the gapmers were evaluated and compared to ISIS 460209 and ISIS 540108.

The gapmers and their motifs are described in Table 95. The internucleoside linkages throughout each modified oligonucleotide are phosphorothioate linkages (P═S). Nucleosides without a subscript are (3-D-2′-deoxyribonucleosides. Nucleosides followed by a subscript “e” indicate 2′-O-methoxyethyl (MOE) modified nucleosides. Nucleosides followed by a subscript “k” indicate 6′-(S)—CH₃ bicyclic nucleosides (e.g. cEt). ^{m}C indicates a 5-methyl cytosine nucleoside. Underlined nucleoside indicates the position on the oligonucleotides opposite to the SNP position, which is position 8 or 9 as counted from the 5′-terminus.

The modified oligonucleotides were tested in vitro. Heterozygous fibroblast GM04022 cell line was used. Cultured GM04022 cells at a density of 25,000 cells per well were transfected using electroporation with 0.12, 0.37, 1.1, 3.3 and 10 μM concentrations of modified oligonucleotides. After a treatment period of approximately 16 hours, RNA was isolated from the cells and mRNA levels were measured by quantitative real-time PCR using ABI assay C_2229297_10 which measures at dbSNP rs362303. The HTT mRNA levels were adjusted according to total RNA content, as measured by RIBOGREEN and the results are presented in Table 96.

The IC₅₀ and selectivity were calculated using methods previously described in Example 41. The IC₅₀ at which each oligonucleotide inhibits the mutant HTT mRNA expression is denoted as ‘mut IC₅₀’. The IC₅₀ at which each oligonucleotide inhibits the wild-type HTT mRNA expression is denoted as ‘wt IC₅₀’. Selectivity was calculated by dividing the IC₅₀ for inhibition of the wild-type HTT versus the IC₅₀ for inhibiting expression of the mutant HTT mRNA.

As illustrated in Table 96, most of the newly designed oligonucleotides showed improvement in selectivity while maintaining potency as compared to 460209.

TABLE 95
Short-gap antisense oligonucleotides
targeting HTT SNP

| | | | Gap | Wing | | SEQ |
|---|---|---|---|---|---|---|
| ISIS | Sequence | | chem- | chemistry | | ID |
| NO. | (5′ to 3′) | Motif | istry | 5′ | 3′ | NO. |
| 460209 | TₑAₖAₖATTGT | 3-9-3 | Full | ekk | kke | 10 |
| | ^{m}CAT^{m}CAₖ^{m}Cₖ^{m}Cₑ | | deoxy | | | |
| 540108 | AₑTₑAₑAₖAₖTTGT | 5-7-5 | Full | eeekk | kkeee | 32 |
| | ^{m}CAT^{m}CₖAₖ^{m}Cₑ^{m}CₑAₑ | | deoxy | | | |
| 556872 | AₑTₑAₑAₑAₖTTGT | 5-7-5 | Full | eeeek | eeeee | 32 |
| | ^{m}CAT^{m}CₑAₑ^{m}Cₑ^{m}CₑAₑ | | deoxy | | | |
| 556873 | AₑTₑAₑAₖAₖTTGT | 5-7-5 | Full | eeekk | eeeee | 32 |
| | ^{m}CAT^{m}CₑAₑ^{m}Cₑ^{m}CₑAₑ | | deoxy | | | |
| 556874 | AₑTₑAₖAₖAₖTTGT | 5-7-5 | Full | eekkk | eeeee | 32 |
| | ^{m}CAT^{m}CₑAₑ^{m}Cₑ^{m}CₑAₑ | | deoxy | | | |
| 568877 | AₑTₖAₖAₖAₖTTGT | 5-7-5 | Full | ekkkk | eeeee | 32 |
| | ^{m}CAT^{m}CₑAₑ^{m}Cₑ^{m}CₑAₑ | | deoxy | | | |
| 568878 | AₖTₖAₖAₖAₖTTGT | 5-7-5 | Full | kkkkk | eeeee | 32 |
| | ^{m}CAT^{m}CₑAₑ^{m}Cₑ^{m}CₑAₑ | | deoxy | | | |

e = 2′-MOE, k = cEt, d = 2′-deoxyribonucleoside

TABLE 96
Comparison of inhibition of HTT mRNA levels and selectivity
of modified oligonucleotides with ISIS 460209 targeting HTT SNP

| | | | | | | Wing | |
|---|---|---|---|---|---|---|---|
| | IC₅₀ (μM) | | Selectivity | | Gap | Chemistry | |
| ISIS NO | Mut | Wt | (wt vs mut) | Motif | chemistry | 5′ | 3′ |
| 460209 | 0.45 | 2.3 | 5.1 | 3-9-3 | Full deoxy | ekk | kke |
| 540108 | 0.25 | 9.5 | 38 | 5-7-5 | Full deoxy | eeekk | kkeee |
| 556872 | 1.0 | 9.9 | 9.9 | 5-7-5 | Full deoxy | eeeek | eeeee |
| 556873 | 0.67 | 3.4 | 5.1 | 5-7-5 | Full deoxy | eeekk | eeeee |
| 556874 | 0.38 | 1.9 | 5.0 | 5-7-5 | Full deoxy | eekkk | eeeee |
| 568877 | 0.44 | 6.2 | 14 | 5-7-5 | Full deoxy | ekkkk | eeeee |
| 568878 | 0.41 | 8.6 | 21 | 5-7-5 | Full deoxy | kkkkk | eeeee |

e = 2′-MOE,
k = cEt,
d = 2′-deoxyribonucleoside

### Example 64

### Short-Gap Chimeric Oligonucleotides Targeting Huntingtin (HTT) Single Nucleotide Polymorphism (SNP)

Additional chimeric antisense oligonucleotides were designed based on 15-mer, ISIS 460209 and 17-mer, ISIS 540108 wherein the central gap region contains nine and seven 2′-deoxynucleosides, respectively. These gapmers were designed by introducing one or more cEt modification(s) at the 3′-end of the central gap region. The gapmers were tested for their ability to selectively inhibit mutant (mut) HTT mRNA expression levels targeting HTT SNP while leaving the expression of the wild-type (wt) intact. The activity and selectivity of the gapmers were evaluated and compared to ISIS 460209 and ISIS 540108.

The gapmers and their motifs are described in Table 97. The internucleoside linkages throughout each modified oligonucleotide are phosphorothioate linkages (P═S). Nucleosides without a subscript are (3-D-2′-deoxyribonucleosides. Nucleosides followed by a subscript “e” indicate 2′-O-methoxyethyl (MOE) modified nucleosides. Nucleosides followed by a subscript “k” indicate 6′-(S)—CH₃ bicyclic nucleosides (e.g. cEt). ^{m}C indicates a 5-methyl cytosine nucleoside. Underlined nucleoside indicates the position on the oligonucleotides opposite to the SNP position, which is position 8 or 9 as counted from the 5′-terminus.

The modified oligonucleotides were tested in vitro. Heterozygous fibroblast GM04022 cell line was used. Cultured GM04022 cells at a density of 25,000 cells per well were transfected using electroporation with 0.12, 0.37, 1.1, 3.3 and 10 μM concentrations of modified oligonucleotides. After a treatment period of approximately 16 hours, RNA was isolated from the cells and mRNA levels were measured by quantitative real-time PCR using ABI assay C_2229297_10 which measures at dbSNP rs362303. The HTT mRNA levels were adjusted according to total RNA content, as measured by RIBOGREEN and the results are presented in Table 98.

The IC₅₀ and selectivity were calculated using methods previously described in Example 41. The IC₅₀ at which each oligonucleotide inhibits the mutant HTT mRNA expression is denoted as ‘mut IC₅₀’. The IC₅₀ at which each oligonucleotide inhibits the wild-type HTT mRNA expression is denoted as ‘wt IC₅₀’. Selectivity was calculated by dividing the IC₅₀ for inhibition of the wild-type HTT versus the IC₅₀ for inhibiting expression of the mutant HTT mRNA.

As illustrated in Table 98, each of the newly designed oligonucleotides showed improvement in selective inhibition of mutant HTT mRNA levels compared to ISIS 460209. Comparable potency was observed for ISIS 568879 and 568880 while a slight loss in potency was observed for ISIS 556875, 556876 and 556877.

TABLE 97
Short-gap antisense oligonucleotides
targeting HTT SNP

| | | | Gap | Wing | | SEQ |
|---|---|---|---|---|---|---|
| ISIS | Sequence | | chem- | chemistry | | ID |
| NO. | (5′ to 3′) | Motif | istry | 5′ | 3′ | NO. |
| 460209 | TₑAₖAₖATTGT | 3-9-3 | Full | ekk | kke | 10 |
| | ^{m}CAT^{m}CAₖ^{m}Cₖ^{m}Cₑ | | deoxy | | | |
| 540108 | AₑTₑAₑAₖAₖTTGT | 5-7-5 | Full | eeekk | kkeee | 32 |
| | ^{m}CAT^{m}CₖAₖ^{m}Cₑ^{m}CₑAₑ | | deoxy | | | |
| 556875 | AₑTₑAₑAₑAₑTTGT | 5-7-5 | Full | eeeee | keeee | 32 |
| | ^{m}CAT^{m}CₖAₑ^{m}Cₑ^{m}CₑAₑ | | deoxy | | | |
| 556876 | AₑTₑAₑAₑAₑTTGT | 5-7-5 | Full | eeeee | kkeee | 32 |
| | ^{m}CAT^{m}CₖAₖ^{m}Cₑ^{m}CₑAₑ | | deoxy | | | |
| 556877 | AₑTₑAₑAₑAₑTTGT | 5-7-5 | Full | eeeee | kkkee | 32 |
| | ^{m}CAT^{m}CₖAₖ^{m}Cₖ^{m}CₑAₑ | | deoxy | | | |
| 568879 | AₑTₑAₑAₑAₑTTGT | 5-7-5 | Full | eeeee | kkkke | 32 |
| | ^{m}CAT^{m}CₖAₖ^{m}Cₖ^{m}CₖAₑ | | deoxy | | | |
| 568880 | AₑTₑAₑAₑAₑTTGT | 5-7-5 | Full | eeeee | kkkkk | 32 |
| | ^{m}CAT^{m}CₖAₖ^{m}Cₖ^{m}CₖAₖ | | deoxy | | | |

e = 2′-MOE, k = cEt, d = 2′-deoxyribonucleoside

TABLE 98
Comparison of inhibition of HTT mRNA levels and selectivity of
modified oligonucleotides with ISIS 460209 targeting HTT SNP

| | | | Selectivity | | | Wing | |
|---|---|---|---|---|---|---|---|
| | IC₅₀ (μM) | | (wt vs | | Gap | Chemistry | |
| ISIS NO | Mut | Wt | mut) | Motif | chemistry | 5′ | 3′ |
| 460209 | 0.45 | 2.3 | 5.1 | 3-9-3 | Full deoxy | ekk | kke |
| 540108 | 0.25 | 9.5 | 38 | 5-7-5 | Full deoxy | eeekk | kkeee |
| 556875 | 1.9 | >9.5 | >5 | 5-7-5 | Full deoxy | eeeee | keeee |
| 556876 | 0.99 | >9.9 | >10 | 5-7-5 | Full deoxy | eeeee | kkeee |
| 556877 | 1.0 | >10 | >10 | 5-7-5 | Full deoxy | eeeee | kkkee |
| 568879 | 0.44 | >10.1 | >23 | 5-7-5 | Full deoxy | eeeee | kkkke |
| 568880 | 0.59 | >10 | >17 | 5-7-5 | Full deoxy | eeeee | kkkkk |

e = 2′-MOE,
k = cEt,
d = 2′-deoxyribonucleoside

### Example 65

### Modified Oligonucleotides Targeting Huntingtin (HTT) Single Nucleotide Polymorphism (SNP)

A series of modified oligonucleotides were designed based on the parent gapmer, ISIS 460209 wherein the central gap region contains nine 2′-deoxyribonucleosides. These modified oligonucleotides were designed by introducing various chemical modifications in the central gap region and were tested for their ability to selectively inhibit mutant (mut) HTT mRNA expression levels targeting SNP while leaving the expression of the wild-type (wt) intact. The activity and selectivity of the modified oligonucleotides were evaluated and compared to the parent gapmer, ISIS 460209.

The modified oligonucleotides were created with a 3-9-3 motif and are described in Table 99. The internucleoside linkages throughout each gapmer are phosphorothioate (P═S) linkages, except for the internucleoside linkage having a subscript “p” which indicates a methyl phosphonate internucleoside linkage (—O—P(CH₃)(═O)—O—). Nucleosides without a subscript are β-D-2′-deoxyribonucleosides. Nucleosides followed by a subscript “e” indicates a 2′-O-methoxyethyl (MOE) modified nucleoside. Nucleosides followed by a subscript “k” indicates a 6′-(S)—CH₃ bicyclic nucleoside (e.g. cEt). ^{m}C indicates a 5-methyl cytosine nucleoside. ^{x}T indicates a 2-thio-thymidine nucleoside. Underlined nucleoside indicates the position on the oligonucleotides opposite to the SNP position, which is position 8 as counted from the 5′-terminus.

The modified oligonucleotides were tested in vitro. Heterozygous fibroblast GM04022 cell line was used (from Coriell Institute). Cultured GM04022 cells at a density of 25,000 cells per well were transfected using electroporation with 0.12, 0.37, 1.1, 3.3 and 10 μM concentrations of modified oligonucleotides. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. The HTT mRNA levels were adjusted according to total RNA content, as measured by RIBOGREEN and the results are presented in Table 100.

The IC₅₀ and selectivity were calculated using methods previously described in Example 41. The IC₅₀ at which each oligonucleotide inhibits the mutant HTT mRNA expression is denoted as ‘mut IC₅₀’. The IC₅₀ at which each oligonucleotide inhibits the wild-type HTT mRNA expression is denoted as ‘wt IC₅₀’. Selectivity was calculated by dividing the IC₅₀ for inhibition of the wild-type HTT versus the IC₅₀ for inhibiting expression of the mutant HTT mRNA.

As illustrated in Table 100, improvement in selectivity with a slight decrease in potency was observed for the newly designed oligonucleotides as compared to ISIS 460209.

TABLE 99
Short-gap antisense oligonucleotides
targeting HTT SNP

| | | | Wing | | SEQ |
|---|---|---|---|---|---|
| ISIS | Sequence | Gap | chemistry | | ID |
| NO. | (5′ to 3′) | chemistry | 5′ | 3′ | NO. |
| 460209 | TₑAₖAₖATTGT | Full deoxy | ekk | kke | 10 |
| | ^{m}CAT^{m}CAₖ^{m}Cₖ^{m}Cₑ | | | | |
| 556845 | TₑAₖAₖA^{x}TTGT | Deoxy/2-Thio | ekk | kke | 10 |
| | ^{m}CAT^{m}CAₖ^{m}Cₖ^{m}Cₑ | | | | |
| 556847 | TₑAₖAₖA^{x}T^{x}TGT | Deoxy/2-Thio | ekk | kke | 10 |
| | ^{m}CAT^{m}CAₖ^{m}Cₖ^{m}Cₑ | | | | |
| 558257 | TₑAₖAₖATTₚGT | Deoxy/Methyl | ekk | kke | 10 |
| | ^{m}CAT^{m}CAₖ^{m}Cₖ^{m}C | Phosphonate | | | |
| 571125 | TₑAₖAₖA^{x}TTₚGT | Deoxy/2-Thio/ | ekk | kke | 10 |
| | ^{m}CAT^{m}CAₖ^{m}Cₖ^{m}Cₑ | Methyl | | | |
| | | Phosphonate | | | |

e = 2′-MOE, k = cEt, d = 2′-deoxyribonucleoside

TABLE 100
Comparison of inhibition of HTT mRNA levels and selectivity
of modified oligonucleotides with ISIS 460209 targeting HTT SNP

| | | | | | Wing | |
|---|---|---|---|---|---|---|
| | IC₅₀ (μM) | | Selectivity | | Chemistry | |
| ISIS NO | Mut | Wt | (wt vs mut) | Gap chemistry | 5′ | 3′ |
| 460209 | 0.56 | 3.8 | 6.8 | Full deoxy | ekk | kke |
| 556845 | 0.98 | >9.8 | >10 | Deoxy/2-Thio | ekk | kke |
| 556847 | 1.3 | >10.4 | >8 | Deoxy/2-Thio | ekk | kke |
| 558257 | 1.7 | >10.2 | >6 | Deoxy/Methyl | ekk | kke |
| | | | | Phosphonate | | |
| 571125 | 1.8 | >10.8 | >6 | Deoxy/2- | ekk | kke |
| | | | | Thio/Methyl | | |
| | | | | Phosphonate | | |

e = 2′-MOE,
k = cEt,
d = 2′-deoxyribonucleoside

### Example 66

### Modified Oligonucleotides Comprising Chemical Modifications in the Central Gap Region Targeting Huntingtin (HTT) Single Nucleotide Polymorphism (SNP)

Additional chimeric antisense oligonucleotides were designed in the same manner as the antisense oligonucleotides described in Example 65. These gapmers were designed by introducing various modifications in the central gap region and were tested for their ability to selectively inhibit mutant (mut) HTT mRNA expression levels targeting SNP while leaving the expression of the wild-type (wt) intact. The activity and selectivity of the modified oligonucleotides were evaluated and compared to the parent gapmer, ISIS 460209.

The modified oligonucleotides and their motifs are described in Table 101. The internucleoside linkages throughout each gapmer are phosphorothioate (P═S) linkages, except for the internucleoside linkage having a subscript “p” which indicates a methyl phosphonate internucleoside linkage (—O—P(CH₃)(═O)—O—). Nucleosides without a subscript are β-D-2′-deoxyribonucleosides. Nucleosides followed by a subscript “e” indicates a 2′-O-methoxyethyl (MOE) modified nucleoside. Nucleosides followed by a subscript “k” indicates a 6′-(S)—CH₃ bicyclic nucleoside (e.g. cEt). ^{m}C indicates a 5-methyl cytosine nucleoside. ^{x}T indicates a 2-thio-thymidine nucleoside. Underlined nucleoside indicates the position on the oligonucleotides opposite to the SNP position, which is position 8 as counted from the 5′-terminus.

The modified oligonucleotides were tested in vitro. Heterozygous fibroblast GM04022 cell line was used (from Coriell Institute). Cultured GM04022 cells at a density of 25,000 cells per well were transfected using electroporation with 0.12, 0.37, 1.1, 3.3 and 10 μM concentrations of modified oligonucleotides. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. The HTT mRNA levels were adjusted according to total RNA content, as measured by RIBOGREEN and the results are presented in Table 102.

The IC₅₀ and selectivity were calculated using methods previously described in Example 41. The IC₅₀ at which each oligonucleotide inhibits the mutant HTT mRNA expression is denoted as ‘mut IC₅₀’. The IC₅₀ at which each oligonucleotide inhibits the wild-type HTT mRNA expression is denoted as ‘wt IC₅₀’. Selectivity was calculated by dividing the IC₅₀ for inhibition of the wild-type HTT versus the IC₅₀ for inhibiting expression of the mutant HTT mRNA.

As illustrated in Table 102, some of the newly designed oligonucleotides showed improvement in selectivity while maintaining potency as compared to 460209.

TABLE 101
Short-gap antisense oligonucleotides targeting HTT SNP

| | | | | Wing | | SEQ |
|---|---|---|---|---|---|---|
| ISIS | Sequence | | Gap | chemistry | | ID |
| NO. | (5′ to 3′) | Motif | chemistry | 5′ | 3′ | NO. |
| 460209 | TₑAₖAₖATTGT | 3-9-3 | Full deoxy | ekk | kke | 10 |
| | ^{m}CAT^{m}CAₖ^{m}Cₖ^{m}Cₑ | | | | | |
| 551429 | TₑAₑAₑAₖTₖTGT | 5-7-3 | Full deoxy | eeekk | kke | 10 |
| | ^{m}CAT^{m}CAₖ^{m}Cₖ^{m}Cₑ | | | | | |
| 571122 | TₑAₑAₑAₖ^{x}TTGT | 4-8-3 | Deoxy/2-Thio | eeek | kke | 10 |
| | ^{m}CAT^{m}CAₖ^{m}Cₖ^{m}Cₑ | | | | | |
| 571123 | TₑAₑAₑAₖTₖTₚGT | 5-7-3 | Deoxy/Methyl | eeekk | kke | 10 |
| | ^{m}CAT^{m}CAₖ^{m}Cₖ^{m}Cₑ | | Phosphonate | | | |
| 571124 | TₑAₑAₑAₖ^{x}TTₚGT | 4-8-3 | Deoxy/2- | eeek | kke | 10 |
| | ^{m}CAT^{m}CAₖ^{m}Cₖ^{m}Cₑ | | Thio/Methyl | | | |
| | | | Phosphonate | | | |
| 579854 | TₑAₑAₑAₖTTₚGT | 4-8-3 | Deoxy/Methyl | eeek | kke | 10 |
| | ^{m}CAT^{m}CAₖ^{m}Cₖ^{m}Cₑ | | Phosphonate | | | |
| 566282 | TₑAₖAₖA_{dx}T_{dx}T_{d}G_{d}T_{d}^{m}C_{d} | 3-9-3 | Deoxy/Methyl | ekk | kke | 10 |
| | A_{d}T_{d}^{m}C_{d}Aₖ^{m}Cₖ^{m}Cₑ | | Phosphonate | | | |

e = 2′-MOE, k = cEt, d = 2′-deoxyribonucleoside

TABLE 102
Comparison of inhibition of HTT mRNA levels and selectivity
of modified oligonucleotides with ISIS 460209 targeting HTT SNP

| | | | Selectivity | | | Wing | |
|---|---|---|---|---|---|---|---|
| ISIS | IC₅₀ (μM) | | (wt vs | | | Chemistry | |
| NO | Mut | Wt | mut) | Motif | Gap chemistry | 5′ | 3′ |
| 460209 | 0.56 | 3.8 | 6.8 | 3-9-3 | Full deoxy | ekk | kke |
| 551429 | 0.50 | >10 | >20 | 5-7-3 | Full deoxy | eeekk | kke |
| 571122 | 1.8 | >10.8 | >6 | 4-8-3 | Deoxy/2-Thio | eeek | kke |
| 571123 | 0.96 | >9.6 | >10 | 5-7-3 | Deoxy/Methyl | eeekk | kke |
| | | | | | Phosphonate | | |
| 571124 | 2.3 | >9.2 | >4 | 4-8-3 | Deoxy/2- | eeek | kke |
| | | | | | Thio/Methyl | | |
| | | | | | Phosphonate | | |
| 579854 | 0.63 | >10.1 | >16 | 4-8-3 | Deoxy/Methyl | eeek | kke |
| | | | | | Phosphonate | | |
| 566282 | 0.51 | 6.3 | 12.4 | 3-9-3 | Deoxy/Methyl | ekk | kke |
| | | | | | Phosphonate | | |

e = 2′-MOE,
k = cEt

### Example 67

### Modified Oligonucleotides Comprising Chemical Modifications in the Central Gap Region Targeting Huntingtin (HTT) Single Nucleotide Polymorphism (SNP)

Additional chimeric antisense oligonucleotides were designed in the same manner as the antisense oligonucleotides described in Example 65. These gapmers were designed by introducing various modifications in the central gap region and were tested for their ability to selectively inhibit mutant (mut) HTT mRNA expression levels targeting SNP while leaving the expression of the wild-type (wt) intact. The activity and selectivity of the modified oligonucleotides were evaluated and compared to the parent gapmer, ISIS 460209.

The modified oligonucleotides and their motifs are described in Table 103. The internucleoside linkages throughout each gapmer are phosphorothioate (P═S) linkages, except for the internucleoside linkage having a subscript “p” which indicates a methyl phosphonate internucleoside linkage (—O—P(CH₃)(═O)—O—). Nucleosides without a subscript are β-D-2′-deoxyribonucleosides. Nucleosides followed by a subscript “e” indicates a 2′-O-methoxyethyl (MOE) modified nucleoside. Nucleosides followed by a subscript “k” indicates a 6′-(S)—CH₃ bicyclic nucleoside (e.g. cEt). ^{m}C indicates a 5-methyl cytosine nucleoside. ^{x}T indicates a 2-thio-thymidine nucleoside. Underlined nucleoside indicates the position on the oligonucleotides opposite to the SNP position, which is position 8 or 9 as counted from the 5′-terminus.

The modified oligonucleotides were tested in vitro. Heterozygous fibroblast GM04022 cell line was used (from Coriell Institute). Cultured GM04022 cells at a density of 25,000 cells per well were transfected using electroporation with 0.12, 0.37, 1.1, 3.3 and 10 μM concentrations of modified oligonucleotides. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. The HTT mRNA levels were adjusted according to total RNA content, as measured by RIBOGREEN and the results are presented in Table 104.

The IC₅₀ and selectivity were calculated using methods previously described in Example 41. The IC₅₀ at which each oligonucleotide inhibits the mutant HTT mRNA expression is denoted as ‘mut IC₅₀’. The IC₅₀ at which each oligonucleotide inhibits the wild-type HTT mRNA expression is denoted as ‘wt IC₅₀’. Selectivity was calculated by dividing the IC₅₀ for inhibition of the wild-type HTT versus the IC₅₀ for inhibiting expression of the mutant HTT mRNA.

As illustrated in Table 104, all but one of the newly designed oligonucleotides showed improvement in selectivity while maintaining potency as compared to ISIS 460209.

TABLE 103
Short-gap antisense oligonucleotides targeting
HTT SNP

| | | | | Wing | | SEQ |
|---|---|---|---|---|---|---|
| ISIS | Sequence | | Gap | chemistry | | ID |
| NO. | (5′ to 3′) | Motif | chemistry | 5′ | 3′ | NO. |
| 460209 | TₑAₖAₖATTGT | 3-9-3 | Full | ekk | kke | 10 |
| | ^{m}CAT^{m}CAₖ^{m}Cₖ^{m}Cₑ | | deoxy | | | |
| 476333 | AₑTₖAₑAₖATTGT | 4-9-4 | Full | ekek | keke | 32 |
| | ^{m}CAT^{m}CAₖ^{m}Cₑ^{m}CₖAₑ | | deoxy | | | |
| 571039 | AₑTₖAₑAₖA^{x}TTGT | 4-9-4 | Deoxy/ | ekek | keke | 32 |
| | ^{m}CAT^{m}CAₖ^{m}Cₑ^{m}CₖAₑ | | 2-Thio | | | |
| 571171 | AₑTₖAₑAₖATTₚGT | 4-9-4 | Deoxy/ | ekek | keke | 32 |
| | ^{m}CAT^{m}CAₖ^{m}Cₑ^{m}CₖAₑ | | Methyl | | | |
| | | | Phospho- | | | |
| | | | nate | | | |
| 571041 | AₑTₖAₑAₖA^{x}TTₚGT | 4-9-4 | Deoxy/2- | ekek | keke | 32 |
| | ^{m}CAT^{m}CAₖ^{m}Cₑ^{m}CₖAₑ | | Thio/ | | | |
| | | | Methyl | | | |
| | | | Phospho- | | | |
| | | | nate | | | |

e = 2′-MOE, k = cEt, d = 2′-deoxyribonucleoside

TABLE 104
Comparison of inhibition of HTT mRNA levels and selectivity of
modified oligonucleotides with ISIS 460209 targeting HTT SNP

| ISIS | IC₅₀ (μM) | | Selectivity | Gap | Wing Chemistry | |
|---|---|---|---|---|---|---|
| NO | Mut | Wt | (wt vs mut) | chemistry | 5′ | 3′ |
| 460209 | 0.56 | 3.8 | 6.8 | Full deoxy | ekk | kke |
| 476333 | 0.56 | 3.4 | 6.1 | Full deoxy | ekek | keke |
| 571039 | 0.34 | >9.9 | >29 | Deoxy/2-Thio | ekek | keke |
| 571171 | 0.54 | >10.3 | >19 | Deoxy/Methyl | ekek | keke |
| | | | | Phosphonate | | |
| 571041 | 0.75 | >9.8 | >13 | Deoxy/2- | ekek | keke |
| | | | | Thio/Methyl | | |
| | | | | Phosphonate | | |

e = 2′-MOE, k = cEt, d = 2′-deoxyribonucleoside

### Example 68

Selectivity in Inhibition of HTT mRNA Levels Targeting SNP by Gap-Interrupted Modified Oligonucleotides

Additional modified oligonucleotides were designed based on the parent gapmer, ISIS 460209 wherein the central gap region contains nine 2′-deoxyribonucleosides. These modified oligonucleotides were designed by introducing one or more modified nucleobase(s) in the central gap region and were tested for their ability to selectively inhibit mutant (mut) HTT mRNA expression levels targeting SNP while leaving the expression of the wild-type (wt) intact. The activity and selectivity of the modified oligonucleotides were evaluated and compared to ISIS 460209.

The modified oligonucleotides were created with a 3-9-3 motif and are described in Table 105. The internucleoside linkages throughout each gapmer are phosphorothioate (P═S) linkages. Nucleosides without a subscript are β-D-2′-deoxyribonucleosides. Nucleosides followed by a subscript “e” indicates a 2′-O-methoxyethyl (MOE) modified nucleoside. Nucleosides followed by a subscript “k” indicates a 6′-(S)—CH₃ bicyclic nucleoside (e.g. cEt). ^{m}C indicates a 5-methyl cytosine nucleoside. ^{x}T indicates a 2-thio-thymidine nucleoside. Underlined nucleoside indicates the position on the oligonucleotides opposite to the SNP position, which is position 8 as counted from the 5′-terminus.

The modified oligonucleotides were tested in vitro. Heterozygous fibroblast GM04022 cell line was used (from Coriell Institute). Cultured GM04022 cells at a density of 25,000 cells per well were transfected using electroporation with 0.12, 0.37, 1.1, 3.3 and 10 μM concentrations of modified oligonucleotides. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. The HTT mRNA levels were adjusted according to total RNA content, as measured by RIBOGREEN.

The IC₅₀ and selectivity were calculated using methods previously described in Example 41. The IC₅₀ at which each oligonucleotide inhibits the mutant HTT mRNA expression is denoted as ‘mut IC₅₀’. The IC₅₀ at which each oligonucleotide inhibits the wild-type HTT mRNA expression is denoted as ‘wt IC₅₀’. Selectivity was calculated by dividing the IC₅₀ for inhibition of the wild-type HTT versus the IC₅₀ for inhibiting expression of the mutant HTT mRNA.

As illustrated in Table 106, ISIS 556845 showed improvement in selectivity and potency as compared to ISIS 460209. ISIS 556847 showed improvement in selectivity with comparable potency while ISIS 556846 showed improvement in potency with comparable selectivity.

TABLE 105
Gap-interrupted modified oligonucleotides
targeting HTT SNP

| | | | Wing | | SEQ |
|---|---|---|---|---|---|
| ISIS | Sequence | Gap | chemistry | | ID |
| NO. | (5′ to 3′) | chemistry | 5′ | 3′ | NO. |
| 460209 | TₑAₖAₖATTGT | Full | ekk | kke | 10 |
| | ^{m}CAT^{m}CAₖ^{m}Cₖ^{m}Cₑ | deoxy | | | |
| 556845 | TₑAₖAₖA^{x}TTGT | Deoxy/ | ekk | kke | 10 |
| | ^{m}CAT^{m}CAₖ^{m}Cₖ^{m}Cₑ | 2-Thio | | | |
| 556846 | TₑAₖAₖAT^{x}TGT | Deoxy/ | ekk | kke | 10 |
| | ^{m}CAT^{m}CAₖ^{m}Cₖ^{m}Cₑ | 2-Thio | | | |
| 556847 | TₑAₖAₖA^{x}T^{x}TGT | Deoxy/ | ekk | kke | 10 |
| | ^{m}CAT^{m}CAₖ^{m}Cₖ^{m}Cₑ | 2-Thio | | | |

e = 2′-MOE, k = cEt, d = 2′-deoxyribonucleoside

TABLE 106
Comparison of inhibition of HTT mRNA levels and
selectivity of gap-interrupted modified oligonucleotides
with ISIS 460209 targeting HTT SNP

| ISIS | IC₅₀ (μM) | | Selectivity | Gap | Wing Chemistry | |
|---|---|---|---|---|---|---|
| NO | Mut | Wt | (wt vs mut) | chemistry | 5′ | 3′ |
| 460209 | 0.30 | 0.99 | 3.3 | Full deoxy | ekk | kke |
| 556845 | 0.13 | 10.01 | >77 | Deoxy/2-Thio | ekk | kke |
| 556846 | 0.19 | 0.48 | 2.5 | Deoxy/2-Thio | ekk | kke |
| 556847 | 0.45 | 9.9 | >22 | Deoxy/2-Thio | ekk | kke |

e = 2′-MOE, k = cEt, d = 2′-deoxyribonucleoside

### Example 69

### Evaluation of Modified Oligonucleotides Targeting HTT SNP—In Vivo Study

Additional modified oligonucleotides were selected and tested for their effects on mutant and wild type HTT protein levels in vivo targeting various SNP sites as illustrated below.

The gapmers and their motifs are described in Table 107. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e” or “k” are sugar modified nucleosides. A subscript “e” indicates a 2′-O-methoxyethyl (MOE) modified nucleoside and a subscript “k” indicates a 6′-(S)—CH₃ bicyclic nucleoside (e.g. cEt).

The gapmer, ISIS 460209 was included in the study as a benchmark oligonucleotide against which the potency and selectivity of the modified oligonuclotides could be compared. A non-allele specific oligonucleotide, ISIS 387898, was used as a positive control.

Hu97/18 mice, the first murine model of HD that fully genetically recapitulates human HD were used in the study. They were generated in Hayden's lab by cross bred BACHD, YAC18 and Hdh (−/−) mice.

Hu97/18 mice were treated with 300 μg of modified oligonucleotides by a single unilateral intracerebroventricular (ICV) bolus injection. This treatment group consisted of 4 animals/oligonucleotide. The control group received a 10 μl bolus injection of sterile PBS and consisted of 4 animals.

Animals were sacrificed at 4 weeks post-injection. The second most anterior 2 mm coronal slab for each brain hemisphere was collected using a 2 mm rodent brain matrix. The remaining portion of the brain was post-fixed in 4% paraformaldehyde, cryoprotected in 30% sucrose and sectioned into 25 pm coronal sections for immunohistochemical analysis.

The HTT protein levels were analyzed by high molecular weight western blot (modified from Invitrogen's NuPAGE Bis-Tris System Protocol). The tissue was homogenized in ice cold SDP lysis buffer. 40 μg of total protein lysate was resolved on 10% low-BIS acrylamide gels (200:1 acrylamide:BIS) with tris-glycine running buffer (25 mM Tris, 190 mM Glycince, 0.1% SDS) containing 10.7 mM β-mercaptoethanol added fresh. Gels were run at 90V for 40 min through the stack, then 190V for 2.5 h, or until the 75 kDa molecular weight marker band was at the bottom of the gel. Proteins were transferred to nitrocellulose at 24V for 2 h with NuPage transfer buffer (Invitrogen: 25 mM Bicine, 25 mM Bis-Tris, 1.025 mM EDTA, 5% MeOH, pH 7.2). Membranes were blocked with 5% milk in PBS, and then blotted for HTT with MAB2166 (1:1000, millipore). Anti-calnexin (Sigma C4731) immunoblotting was used as loading control. Proteins were detected with IR dye 800CW goat anti-mouse (Rockland 610-131-007) and AlexaFluor 680 goat anti-rabbit (Molecular Probes A21076)-labeled secondary antibodies, and the LiCor Odyssey Infrared Imaging system.

The results in Table 108 are presented as the average percent of HTT protein levels for each treatment group, normalized to PBS-treated control and is denoted as “% UTC”. The percent of mutant HTT protein levels is denoted as “mut”. The percent of wild-type HTT protein levels is denoted as “wt”. Selectivity was also evaluated and measured by dividing the percent of wild-type HTT protein levels vs. the percent of the mutant HTT protein levels.

As illustrated in Table 108, treatment with the newly designed oligonucleotides, ISIS 476333 and 460085 showed improvement in potency and selectivity in inhibiting mutant HTT protein levels as compared to the parent gapmer, 460209. Comparable or a slight loss in potency and/or selectivity was observed for the remaining oligonucleotides.

TABLE 107
Modified oligonucleotides targeting HTT rs7685686,
rs4690072 and rs363088 in Hu97/18 mice

| | | | Wing | | SEQ |
|---|---|---|---|---|---|
| ISIS | | | Chemistry | | ID |
| NO | Sequence (5′ to 3′) | Motif | 5′ | 3′ | NO. |
| 387898 | CₑTₑCₑGₑAₑCTAAAGCAGGAₑTₑTₑTₑCₑ | 5-10-5 | e5 | e5 | 79 |
| 460209 | TₑAₖAₖATTGTCATCAₖCₖCₑ | 3-9-3 | ekk | kke | 10 |
| 435879 | AeAₑTₑAₑAₑATTGTCATCAₑCₑCₑAₑGₑ | 5-9-5 | e5 | e5 | 80 |
| 476333 | AₑTₖAₑAₖATTGTCATCAₖCₑCkAₑ | 4-9-4 | ekek | keke | 32 |
| 435874 | CₑAₑCₑAₑGₑTGCTACCCAAₑCₑCₑTₑTₑ | 5-9-5 | e5 | e5 | 81 |
| 435871 | TₑCₑAₑCₑAₑGCTATCTTCTₑCₑAₑTₑCₑ | 5-9-5 | e5 | e5 | 82 |
| 460085 | AₑTₑAₑAₑAₑTTGTCATCₑAₑCₑCₑAₑ | 5-7-5 | e5 | e5 | 32 |

e = 2′-MOE (e.g. e5 = eeeee), k = cEt

TABLE 108
Effects of modified oligonucleotides on mutant and
wild type HTT protein levels in Hu97/18 mice

| | | Dosage | % UTC | | Selectivity |
|---|---|---|---|---|---|
| ISIS NO | SNP site | (μg) | mut | wt | (wt vs mut) |
| PBS | — | 300 | 100 | 100 | 1 |
| 387898 | — | 300 | 23.76 | 25.66 | 1 |
| 460209 | rs7685686 | 300 | 18.16 | 48.99 | 2.7 |
| 435879 | rs7685686 | 300 | 41.48 | 73.11 | 1.8 |
| 476333 | rs7685686 | 300 | 6.35 | 22.05 | 3.5 |
| 460085 | rs7685686 | 300 | 2.9 | 40.1 | 13.8 |
| 435874 | rs4690072 | 300 | 44.18 | 76.63 | 1.7 |
| 435871 | rs363088 | 300 | 33.07 | 89.30 | 2.7 |

### Example 70

### Evaluation of ISIS 435871 in Central Nervous System (CNS) Targeting HTT Rs363088—In Vivo Study

A modified oligonucleotide from Example 68, ISIS 435871 was selected and tested for its effects on mutant and wild type HTT protein levels in the CNS in vivo targeting rs363088.

Hu97/18 mouse was treated with 300 μg of ISIS 435871 by a single unilateral intracerebroventricular (ICV) bolus injection. The animal was sacrificed at 4 weeks post-injection. Regional CNS structures were then micro-dissected including bilateral samples from the most anterior portion of cortex (Cortex 1), an intermediate section of cortex (Cortex 2), the most posterior section of cortex (Cortex 3), the striatum, the hippocampus, the cerebellum, and a 1 cm section of spinal cord directly below the brain stem. Tissue was homogenized and assessed for mutant and wild-type HTT levels by Western blotting using the procedures as described in Example 69. The results are presented below. As no untreated or vehicle treated control is shown, HTT intensity of each allele is expressed as a ratio of calnexin loading control intensity. The ratio of the mutant HTT to the wt HTT in the treated animal was determined and is denoted as “wt/mut”. Having a ratio higher than 1 is indicative of allele-specific silencing.

As illustrated in Table 109, a single unilateral ICV bolus injection of the modified antisense oligonucleotide showed selective HTT silencing throughout the CNS except in the cerebellum, where the antisense oligonucleotide did not distribute evenly.

TABLE 109
Effects of ISIS 435871 on mutant and wild type HTT protein
levels in CNS targeting rs363088 in Hu97/18 mice

| | HTT intensity/calnexin intensity | | |
|---|---|---|---|
| Tissue | wt | mut | wt/mut |
| Cortex 1 | 0.032 | 0.014 | 2.29 |
| Cortex 2 | 0.027 | 0.009 | 3 |
| Cortex 3 | 0.023 | 0.007 | 3.29 |
| Striatum | 0.030 | 0.012 | 2.5 |
| Hippocampus | 0.016 | 0.006 | 2.67 |
| Cerebellum | 0.023 | 0.019 | 1.21 |
| Spinal Cord | 0.014 | 0.007 | 2 |

### Example 71

### Evaluation of Modified Oligonucleotides Targeting HTT Rs7685686—In Vivo Study

Several modified oligonucleotides from Examples 43, 51, 52, 53 and 66 were selected and tested for their effects on mutant and wild type HTT protein levels in vivo targeting HTT rs7685686.

The gapmer, ISIS 460209 was included in the study as a benchmark oligonucleotide against which the potency and selectivity of the modified oligonuclotides could be compared.

Hu97/18 mice were treated with 300 μg of modified oligonucleotides by a single unilateral intracerebroventricular (ICV) bolus injection. This treatment group consisted of 4 animals/oligonucleotide. The control group received a 10 μl bolus injection of sterile PBS and consisted of 4 animals.

Animals were sacrificed at 4 weeks post-injection. The second most anterior 2 mm coronal slab for each brain hemisphere was collected using a 2 mm rodent brain matrix. The HTT protein levels were analyzed in the same manner as described in Example 69 and the results are presented below.

The results in Table 110 are presented as the average percent of HTT protein levels for each allele and treatment group, normalized to PBS-treated control and is denoted as “% UTC”. The percent of mutant HTT protein levels is denoted as “mut”. The percent of wild-type HTT protein levels is denoted as “wt”.

As shown in Table 110, each of the newly designed oligonucleotides showed improvement in selective inhibition of mutant HTT protein levels as compared to ISIS 460209. ISIS 550913 and 540095 showed improvement in potency while the remaining modified oligonucleotides showed comparable or a slight decrease in potency as compared to the parent gapmer.

TABLE 110
Effects of modified oligonucleotides on mutant and wild type
HTT protein levels targeting rs7685686 in Hu97/18 mice

| | | | | Wing | | | SEQ |
|---|---|---|---|---|---|---|---|
| ISIS | % UTC | | | chemistry | | Gap | ID |
| NO | mut | wt | Motif | 5′ | 3′ | chemistry | NO |
| PBS | 100 | 100 | — | — | — | — | — |
| 460209 | 18.16 | 48.99 | 3-9-3 | ekk | kke | Full deoxy | 10 |
| 550913 | 9.31 | 34.26 | 5-9-5 | kkekk | kkekk | Full deoxy | 27 |
| 540095 | 12.75 | 106.05 | 2-9-4 | ek | kkke | Full deoxy | 65 |
| 551429 | 19.07 | 108.31 | 5-7-3 | eeekk | kke | Full deoxy | 10 |
| 540094 | 24.68 | 87.56 | 2-9-4 | ek | kkke | Full deoxy | 67 |
| 540096 | 24.89 | 98.26 | 2-9-4 | ek | kkke | Full deoxy | 68 |
| 540108 | 28.34 | 85.62 | 5-7-5 | eeekk | kkeee | Full deoxy | 23 |

e = 2′-MOE, k = cEt

### Example 72

### Evaluation of Modified Oligonucleotides Targeting HTT Rs7685686—In Vivo Study

Several modified oligonucleotides selected from Examples 57, 58, 61 and 62 were tested and evaluated for their effects on mutant and wild type HTT protein levels in vivo targeting HTT rs7685686.

Hu97/18 mice were treated with 300 μg of modified oligonucleotides by a single unilateral intracerebroventricular (ICV) bolus injection and the control group received a 10 μl bolus injection of sterile PBS. Each treatment group consisted of 4 animals.

Animals were sacrificed at 4 weeks post-injection. The second most anterior 2 mm coronal slab for each brain hemisphere was collected using a 2 mm rodent brain matrix. The HTT protein levels were analyzed in the same manner as described in Example 69. The in vivo study for ISIS 575008 and 571069 marked with an asterisk (*) was performed independently and the results are presented below.

The results in Table 111 are presented as the average percent of HTT protein levels for each allele and treatment group, normalized to PBS-treated control and is denoted as “% UTC”. The percent of mutant HTT protein levels is denoted as “mut”. The percent of wild-type HTT protein levels is denoted as “wt”.

As illustrated in Table 111, selective inhibition of mut HTT protein levels was achieved with the newly designed oligonucleotide treatment as compared to PBS treated control.

TABLE 111
Effects of modified oligonucleotides on mutant and wild type
HTT protein levels targeting rs7685686 in Hu97/18 mice

| | | | | Wing | | | SEQ |
|---|---|---|---|---|---|---|---|
| ISIS | % UTC | | | chemistry | | Gap | ID |
| NO | mut | wt | Motif | 5′ | 3′ | chemistry | NO |
| PBS | 100 | 100 | — | — | — | — | — |
| 575007 | 26.9 | 104.5 | 3-9-3 | ekk | kke | Deoxy/cEt | 10 |
| 575008* | 21.7 | 105.9 | 5-7-3 | ekkkk | kke | Deoxy/cEt | 10 |
| 566267 | 32.8 | 109.3 | 3-9-3 | ekk | kke | Deoxy/F- | 10 |
| | | | | | | HNA | |
| 571036 | 30.3 | 103.3 | 6-7-4 | ekekek | keke | Full deoxy | 32 |
| 571037 | 32.8 | 111.9 | 6-7-4 | eeeekk | keke | Full deoxy | 32 |
| 571069* | 29.4 | 109.8 | 6-7-4 | eeeekk | kkee | Full deoxy | 32 |

e = 2′-MOE, k = cEt

### Example 73

### Evaluation of Modified Oligonucleotides Targeting HTT Rs7685686—In Vivo Dose Response Study

ISIS 476333, 435871, 540108, 575007 and 551429 from previous examples were selected and evaluated at various doses for their effect on mutant and wild type HTT protein levels in vivo targeting HTT rs7685686.

Hu97/18 mice were treated with various doses of modified oligonucleotides as presented in Table 112 by a single unilateral intracerebroventricular (ICV) bolus injection. This treatment group consisted of 4 animals/oligonucleotide. The control group received a 10 μl bolus injection of sterile PBS and consisted of 4 animals.

Animals were sacrificed at 4 weeks post-injection. The second most anterior 2 mm coronal slab for each brain hemisphere was collected using a 2 mm rodent brain matrix. The HTT protein levels were analyzed in the same manner as described in Example 69. The dose response study was performed independently for each modified oligonucleotide and the results are presented below.

The results in Table 112 are presented as the average percent of HTT protein levels for each allele and treatment group, normalized to PBS-treated control and is denoted as “% UTC”. The percent of mutant HTT protein levels is denoted as “mut”. The percent of wild-type HTT protein levels is denoted as “wt”.

As illustrated in Table 112, selective inhibition of mut HTT protein levels was achieved in a dose-dependent manner for the newly designed oligonucleotides.

TABLE 112
Dose-dependent effect of modified oligonucleotides on mutant and
wild type HTT protein levels targeting rs7685686 in Hu97/18 mice

| | Dosage | % UTC | | | SEQ |
|---|---|---|---|---|---|
| ISIS NO | (μg) | mut | wt | Motif | ID NO. |
| PBS | 0 | 100 | 100 | — | |
| 476333 | 50 | 48.7 | 115 | 4-9-4 | 32 |
| | 150 | 23.1 | 53.3 | (ekek-d9-keke) | |
| | 300 | 8.8 | 36.7 | | |
| 435871 | 75 | 114 | 118 | 5-9-5 | 82 |
| | 150 | 47.3 | 80.3 | (e5-d9-e5) | |
| | 300 | 33 | 89.3 | | |
| | 500 | 36 | 97.5 | | |
| 540108 | 75 | 30.5 | 71.7 | 5-7-5 | 32 |
| | 150 | 22 | 81 | (eeekk-d7-kkeee) | |
| | 300 | 8.6 | 59.6 | | |
| 575007 | 150 | 41.5 | 110.7 | 3-9-3 | 10 |
| | 300 | 29 | 119.4 | (ekk-d-k-d7-kke) | |
| | | | | (deoxy gap | |
| | | | | interrupted with cEt) | |
| 551429 | 75 | 58 | 101.3 | 5-7-3 | 10 |
| | 150 | 36.2 | 110.4 | (eeekk-d7-kke) | |
| | 300 | 19.7 | 107.8 | | |

e = 2′-MOE (e.g. e5 = eeeee), k = cEt, d = 2′-deoxyribonucleoside

### Example 74

### Modified Oligonucleotides Targeting Huntingtin (HTT) Single Nucleotide Polymorphism (SNP)

A series of modified oligonucleotides was designed based on a parent gapmer, ISIS 460209, wherein the central gap region contains nine β-D-2′-deoxyribonucleosides. The modified oligonucleotides were designed by introducing a 5′-(R)-Me DNA modification within the central gap region. The 5′-(R)-Me DNA containing oligonucleotides were tested for their ability to selectively inhibit mutant (mut) HTT mRNA expression levels targeting rs7685686 while leaving the expression of the wild-type (wt) intact. The potency and selectivity of the modified oligonucleotides were evaluated and compared to ISIS 460209.

The position on the oligonucleotides opposite to the SNP position, as counted from the 5′-terminus is position 8.

The modified oligonucleotides were created with a 3-9-3 motif and are described in Table 113. The internucleoside linkages throughout each gapmer are phosphorothioate (P═S) linkages. Nucleosides followed by a subscript “d” are β-D-2′-deoxyribonucleosides. Nucleosides followed by a subscript “e” indicates a 2′-O-methoxyethyl (MOE) modified nucleoside. Nucleosides followed by a subscript “k” indicates a 6′-(S)—CH₃ bicyclic nucleoside (e.g. cEt). Nucleosides followed by a subscript “z” indicates a 5′-(R)-Me DNA. “^{m}C” indicates a 5-methyl cytosine nucleoside.

The modified oligonucleotides were tested in vitro. Heterozygous fibroblast GM04022 cell line was used. Cultured GM04022 cells at a density of 25,000 cells per well were transfected using electroporation with a single dose at 2 μM concentration of the modified oligonucleotide. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. The HTT mRNA levels were adjusted according to total RNA content, as measured by RIBOGREEN.

The IC₅₀s and selectivities as expressed in “fold” were measured and calculated using methods described previously in Example 41. As illustrated in Table 114, treatment with the newly designed oligonucleotides showed comparable or a slight increase in potency and/or selectivity as compared to ISIS 460209.

TABLE 113
Gap-interrupted oligonucleotides comprising 5′-(R)-Me DNA
targeting HTT SNP

| | | | Wing | | SEQ |
|---|---|---|---|---|---|
| ISIS | | | chemistry | | ID |
| NO. | Sequence (5′ to 3′) | Gap chemistry | 5′ | 3′ | NO. |
| 460209 | TₑAₖAₖA_{d}T_{d}T_{d}G_{d}T_{d}^{m}C_{d}A_{d}T_{d}^{m}C_{d}Aₖ^{m}Cₖ^{m}Cₑ | Full deoxy | ekk | kke | 10 |
| 556848 | TₑAₖAₖA_{z}T_{d}T_{d}G_{d}T_{d}^{m}C_{d}A_{d}T_{d}^{m}C_{d}Aₖ^{m}Cₖ^{m}Cₑ | Deoxy/5′-(R)-Me DNA | ekk | kke | 10 |
| 556849 | TₑAₖAₖA_{d}T_{z}T_{d}G_{d}T_{d}^{m}C_{d}A_{d}T_{d}^{m}C_{d}Aₖ^{m}Cₖ^{m}Cₑ | Deoxy/5′-(R)-Me DNA | ekk | kke | 10 |
| 556850 | TₑAₖAₖA_{d}T_{d}T_{z}G_{d}T_{d}^{m}C_{d}A_{d}T_{d}^{m}C_{d}Aₖ^{m}Cₖ^{m}Cₑ | Deoxy/5′-(R)-Me DNA | ekk | kke | 10 |

e = 2′-MOE, k = cEt

TABLE 114
Comparison of inhibition of HTT mRNA levels and selectivity of gap-
interrupted oligonucleotides with ISIS 460209 targeting HTT SNP

| | IC₅₀ | | | | Wing | |
|---|---|---|---|---|---|---|
| ISIS | (μM) | | Selectivity | Gap | chemistry | |
| NO. | Mut | Wt | (wt vs mut) | chemistry | 5′ | 3′ |
| 460209 | 0.30 | 0.99 | 3.3 | Full deoxy | ekk | kke |
| 556848 | 0.15 | 0.6 | 4.0 | Deoxy/5′-(R)- | ekk | kke |
| | | | | Me DNA | | |
| 556849 | 0.16 | 0.46 | 2.9 | Deoxy/5′-(R)- | ekk | kke |
| | | | | Me DNA | | |
| 556850 | 0.33 | 0.96 | 2.9 | Deoxy/5′-(R)- | ekk | kke |
| | | | | Me DNA | | |

e = 2′-MOE, k = cEt

### Example 75

### Modified Oligonucleotides Comprising 5′-(R)- or 5′-(S)-Me DNA Modification Targeting HTT SNP

A series of modified oligonucleotides was designed based on a parent gapmer, ISIS 460209, wherein the central gap region contains nine β-D-2′-deoxyribonucleosides. The modified oligonucleotides were designed by introducing 5′-(S)- or 5′-(R)-Me DNA modification slightly upstream or downstream (i.e. “microwalk”) within the central gap region. The gapmers were created with a 3-9-3 motif and were tested for their ability to selectively inhibit mutant (mut) HTT mRNA expression. The potency and selectivity of the modified oligonucleotides were evaluated and compared to ISIS 460209.

The position on the oligonucleotides opposite to the SNP position, as counted from the 5′-terminus is position 8.

The modified oligonucleotides and their motifs are described in Table 115. The internucleoside linkages throughout each gapmer are phosphorothioate (P═S) linkages. Nucleosides followed by a subscript “d” are β-D-2′-deoxyribonucleosides. Nucleosides followed by a subscript “e” indicates a 2′-O-methoxyethyl (MOE) modified nucleoside. Nucleosides followed by a subscript “k” indicates a 6′-(S)—CH₃ bicyclic nucleoside (e.g. cEt). Nucleosides followed by a subscript “v” indicates a 5′-(S)-Me DNA. Nucleosides followed by a subscript “z” indicates a 5′-(R)-Me DNA. “^{m}C” indicates a 5-methyl cytosine nucleoside.

The modified oligonucleotides were tested in vitro. Heterozygous fibroblast GM04022 cell line was used. Cultured GM04022 cells at a density of 25,000 cells per well were transfected using electroporation with 0.1, 0.4, 1.1, 3.3 and 10 μM concentrations of modified oligonucleotides. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 uL 2×PCR buffer, 101 uL primers (300 uM from ABI), 1000 uL water and 40.4 uL RT MIX. To each well was added 15 uL of this mixture and 5 uL of purified RNA. The mutant and wild-type HTT mRNA levels were measured simultaneously by using two different fluorophores, FAM for mutant allele and VIC for wild-type allele. The HTT mRNA levels were adjusted according to total RNA content, as measured by RIBOGREEN and the results are presented below.

The IC₅₀s and selectivities as expressed in “fold” were measured and calculated using methods described previously in Example 41. The results in Table 116 demonstrated that each of the newly designed oligonucleotides comprising 5′-(S)- or 5′-(R)-Me DNA within the central gap region achieved improvement in potency and selectivity as compared to the parent gapmer, ISIS 460209.

TABLE 115
Gap-interrupted oligonucleotides comprising 5′-(S)- or
5′-(R)-Me DNA targeting HTT SNP

| | | | | Wing | | SEQ |
|---|---|---|---|---|---|---|
| ISIS | | | Gap | Chemistry | | ID |
| NO | Sequence (5′ to 3′) | Motif | Chemistry | 5′ | 3′ | NO |
| 460209 | TₑAₖAₖA_{d}T_{d}T_{d}G_{d}T_{d}^{m}C_{d}A_{d}T_{d}^{m}C_{d}Aₖ^{m}Cₖ^{m}Cₑ | 3-9-3 | Full deoxy | ekk | kke | 10 |
| 589429 | TₑAₖAₖA_{d}TᵥT_{d}G_{d}T_{d}^{m}C_{d}A_{d}T_{d}^{m}C_{d}Aₖ^{m}Cₖ^{m}Cₑ | 3-9-3 | Deoxy/5′-(S)- | ekk | kke | 10 |
| | | | Me DNA | | | |
| 589430 | TₑAₖAₖA_{d}T_{d}TᵥG_{d}T_{d}^{m}C_{d}A_{d}T_{d}^{m}C_{d}Aₖ^{m}Cₖ^{m}Cₑ | 3-9-3 | Deoxy/5′-(S)- | ekk | kke | 10 |
| | | | Me DNA | | | |
| 589431 | TₑAₖAₖA_{d}T_{d}T_{d}G_{d}Tᵥ^{m}C_{d}A_{d}T_{d}^{m}C_{d}Aₖ^{m}Cₖ^{m}Cₑ | 3-9-3 | Deoxy/5′-(S)- | ekk | kke | 10 |
| | | | Me DNA | | | |
| 589432 | TₑAₖAₖA_{d}T_{d}T_{d}G_{d}T_{d}^{m}C_{d}A_{d}Tᵥ^{m}C_{d}Aₖ^{m}Cₖ^{m}Cₑ | 3-9-3 | Deoxy/5′-(S)- | ekk | kke | 10 |
| | | | Me DNA | | | |
| 594588 | TₑAₖAₖA_{d}TᵥTᵥG_{d}T_{d}^{m}C_{d}A_{d}T_{d}^{m}C_{d}Aₖ^{m}Cₖ^{m}Cₑ | 3-9-3 | Deoxy/5′-(S)- | ekk | kke | 10 |
| | | | Me DNA | | | |
| 556848 | TₑAₖAₖA_{z}T_{d}T_{d}G_{d}T_{d}^{m}C_{d}A_{d}T_{d}^{m}C_{d}Aₖ^{m}Cₖ^{m}Cₑ | 3-9-3 | Deoxy/5′-(R)- | ekk | kke | 10 |
| | | | Me DNA | | | |
| 556849 | TₑAₖAₖA_{d}T_{z}T_{d}G_{d}T_{d}^{m}C_{d}A_{d}T_{d}^{m}C_{d}Aₖ^{m}Cₖ^{m}Cₑ | 3-9-3 | Deoxy/5′-(R)- | ekk | kke | 10 |
| | | | Me DNA | | | |
| 556850 | TₑAₖAₖA_{d}T_{d}T_{z}G_{d}T_{d}^{m}C_{d}A_{d}T_{d}^{m}C_{d}Aₖ^{m}Cₖ^{m}Cₑ | 3-9-3 | Deoxy/5′-(R)- | ekk | kke | 10 |
| | | | Me DNA | | | |
| 539558 | TₑAₖAₖA_{d}T_{d}T_{d}G_{d}T_{z}^{m}C_{d}A_{d}T_{d}^{m}C_{d}Aₖ^{m}Cₖ^{m}Cₑ | 3-9-3 | Deoxy/5′-(R)- | ekk | kke | 10 |
| | | | Me DNA | | | |
| 594160 | TₑAₖAₖA_{d}T_{d}T_{d}G_{d}T_{d}^{m}C_{z}A_{d}T_{d}^{m}C_{d}Aₖ^{m}Cₖ^{m}Cₑ | 3-9-3 | Deoxy/5′-(R)- | ekk | kke | 10 |
| | | | Me DNA | | | |
| 594161 | TₑAₖAₖA_{d}T_{d}T_{d}G_{d}T_{d}^{m}C_{d}A_{z}T_{d}^{m}C_{d}Aₖ^{m}Cₖ^{m}Cₑ | 3-9-3 | Deoxy/5′-(R)- | ekk | kke | 10 |
| | | | Me DNA | | | |
| 589433 | TₑAₖAₖA_{d}T_{d}T_{d}G_{d}T_{d}^{m}C_{d}A_{d}T_{z}^{m}C_{d}Aₖ^{m}Cₖ^{m}Cₑ | 3-9-3 | Deoxy/5′-(R)- | ekk | kke | 10 |
| | | | Me DNA | | | |
| 594162 | TₑAₖAₖA_{d}T_{d}T_{d}G_{d}T_{d}^{m}C_{d}A_{d}T_{d}^{m}C_{z}Aₖ^{m}Cₖ^{m}Cₑ | 3-9-3 | Deoxy/5′-(R)- | ekk | kke | 10 |
| | | | Me DNA | | | |
| 594589 | TₑAₖAₖA_{d}T_{z}T_{z}G_{d}T_{d}^{m}C_{d}A_{d}T_{d}^{m}C_{d}Aₖ^{m}Cₖ^{m}Cₑ | 3-9-3 | Deoxy/5′-(R)- | ekk | kke | 10 |
| | | | Me DNA | | | |

e = 2′-MOE; k = cEt

TABLE 116
Comparison of inhibition of HTT mRNA levels and selectivity of gap-
interrupted oligonucleotides with ISIS 460209 targeting HTT SNP

| ISIS | IC₅₀ (μM) | | Selectivity | | | Wing Chemistry | |
|---|---|---|---|---|---|---|---|
| NO. | Mut | Wt | (wt vs. mut) | Motif | Gap Chemistry | 5′ | 3′ |
| 460209 | 1.2 | 1.4 | 1.2 | 3-9-3 | Full deoxy | ekk | kke |
| 589429 | 0.22 | 3.3 | 15 | 3-9-3 | Deoxy/5′-(S)-Me DNA | ekk | kke |
| 589430 | 0.22 | >10 | >45.5 | 3-9-3 | Deoxy/5′-(S)-Me DNA | ekk | kke |
| 589431 | 0.16 | 1.9 | 11.9 | 3-9-3 | Deoxy/5′-(S)-Me DNA | ekk | kke |
| 589432 | 0.23 | >10 | >43.5 | 3-9-3 | Deoxy/5′-(S)-Me DNA | ekk | kke |
| 594588 | 0.81 | >10 | >12.3 | 3-9-3 | Deoxy/5′-(S)-Me DNA | ekk | kke |
| 556848 | 0.16 | 1.8 | 11.3 | 3-9-3 | Deoxy/5′-(R)-Me DNA | ekk | kke |
| 556849 | 0.14 | 1.1 | 7.9 | 3-9-3 | Deoxy/5′-(R)-Me DNA | ekk | kke |
| 556850 | 0.22 | 1.7 | 7.7 | 3-9-3 | Deoxy/5′-(R)-Me DNA | ekk | kke |
| 539558 | 0.38 | 3.8 | 10 | 3-9-3 | Deoxy/5′-(R)-Me DNA | ekk | kke |
| 594160 | 0.28 | 3.3 | 11.8 | 3-9-3 | Deoxy/5′-(R)-Me DNA | ekk | kke |
| 594161 | 0.28 | >10 | >35.7 | 3-9-3 | Deoxy/5′-(R)-Me DNA | ekk | kke |
| 589433 | 0.27 | 4.4 | 16.3 | 3-9-3 | Deoxy/5′-(R)-Me DNA | ekk | kke |
| 594162 | 0.27 | 3.5 | 13.0 | 3-9-3 | Deoxy/5′-(R)-Me DNA | ekk | kke |
| 594589 | 0.48 | 4.4 | 9.2 | 3-9-3 | Deoxy/5′-(R)-Me DNA | ekk | kke |

e = 2′-MOE; k = cEt

### Example 76

Inhibition of HTT mRNA Levels Targeting SNP by Modified Oligonucleotides

Additional modified oligonucleotides were designed in a similar manner as the antisense oligonucleotides described in Example 75. Various chemical modifications were introduced slightly upstream or downstream (i.e. “microwalk”) within the central gap region. The gapmers were created with a 3-9-3 motif and were tested for their ability to selectively inhibit mutant (mut) HTT mRNA expression. The position on the oligonucleotides opposite to the SNP position, as counted from the 5′-terminus is position 8. The potency and selectivity of the modified oligonucleotides were evaluated and compared to ISIS 460209.

The modified oligonucleotides and their motifs are described in Table 117. The internucleoside linkages throughout each gapmer are phosphorothioate (P═S) linkages. Nucleosides followed by a subscript “d” are β-D-2′-deoxyribonucleosides. Nucleosides followed by a subscript “e” indicates a 2′-O-methoxyethyl (MOE) modified nucleoside. Nucleosides followed by a subscript “k” indicates a 6′-(S)—CH₃ bicyclic nucleoside (e.g. cEt). Nucleosides followed by a subscript “b” indicates a 5′-(R)-allyl DNA. Nucleosides followed by a subscript “c” indicates a 5′-(S)-allyl DNA. Nucleosides followed by a subscript “g” indicates a 5′-(R)-hydroxyethyl DNA. Nucleosides followed by a subscript “i” indicates a 5′-(S)-hydroxyethyl DNA. “^{m}C” indicates a 5-methyl cytosine nucleoside.

The modified oligonucleotides were tested in vitro using heterozygous fibroblast GM04022 cell line. The transfection method and analysis of HTT mRNA levels adjusted according to total RNA content, as measured by RIBOGREEN were performed in the same manner as described in Example 76. The IC₅₀s and selectivities as expressed in “fold” were measured and calculated using methods described previously and the results are shown below. As presented in Table 118, several modified oligonucleotides achieved greater than 4.5 fold selectivity in inhibiting mutant HTT mRNA levels and, therefore, are more selective than ISIS 460209.

TABLE 117
Gap-interrupted oligonucleotides comprising 5′-substituted DNA
targeting HTT SNP

| | | | | Wing | | SEQ |
|---|---|---|---|---|---|---|
| ISIS | | | Gap Chemistry | Chemisty | | ID |
| NO | Sequence (5′ to 3′) | Motif | (mod position) | 5′ | 3′ | NO |
| 460209 | TₑAₖAₖA_{d}T_{d}T_{d}G_{d}T_{d}^{m}C_{d}A_{d}T_{d}^{m}C_{d}Aₖ^{m}Cₖ^{m}Cₑ | 3-9-3 | Full deoxy | ekk | kke | 10 |
| 589414 | TₑAₖAₖA_{d}T_{b}T_{d}G_{d}T_{d}^{m}C_{d}A_{d}T_{d}^{m}C_{d}Aₖ^{m}Cₖ^{m}Cₑ | 3-9-3 | Deoxy/5′-(R)- | ekk | kke | 10 |
| | | | allyl DNA | | | |
| | | | (pos 5) | | | |
| 589415 | TₑAₖAₖA_{d}T_{d}T_{b}G_{d}T_{d}^{m}C_{d}A_{d}T_{d}^{m}C_{d}Aₖ^{m}Cₖ^{m}Cₑ | 3-9-3 | Deoxy/5′-(R)- | ekk | kke | 10 |
| | | | allyl DNA | | | |
| | | | (pos 6) | | | |
| 589416 | TₑAₖAₖA_{d}T_{d}T_{d}G_{d}T_{b}^{m}C_{d}A_{d}T_{d}^{m}C_{d}Aₖ^{m}Cₖ^{m}Cₑ | 3-9-3 | Deoxy/5′-(R)- | ekk | kke | 10 |
| | | | allyl DNA | | | |
| | | | (pos 8) | | | |
| 589417 | TₑAₖAₖA_{d}T_{d}T_{d}G_{d}T_{d}^{m}C_{d}A_{d}T_{b}^{m}C_{d}Aₖ^{m}Cₖ^{m}Cₑ | 3-9-3 | Deoxy/5′-(R)- | ekk | kke | 10 |
| | | | allyl DNA | | | |
| | | | (pos 11) | | | |
| 589418 | TₑAₖAₖA_{d}T_{c}T_{d}G_{d}T_{d}^{m}C_{d}A_{d}T_{d}^{m}C_{d}Aₖ^{m}Cₖ^{m}Cₑ | 3-9-3 | Deoxy/5′-(S)- | ekk | kke | 10 |
| | | | allyl DNA | | | |
| | | | (pos 5) | | | |
| 589419 | TₑAₖAₖA_{d}T_{d}T_{c}G_{d}T_{d}^{m}C_{d}A_{d}T_{d}^{m}C_{d}Aₖ^{m}Cₖ^{m}Cₑ | 3-9-3 | Deoxy/5′-(S)- | ekk | kke | 10 |
| | | | allyl DNA | | | |
| | | | (pos 6) | | | |
| 589420 | TₑAₖAₖA_{d}T_{d}T_{d}G_{d}T_{c}^{m}C_{d}A_{d}T_{d}^{m}C_{d}Aₖ^{m}Cₖ^{m}Cₑ | 3-9-3 | Deoxy/5′-(S)- | ekk | kke | 10 |
| | | | allyl DNA | | | |
| | | | (pos 8) | | | |
| 589421 | TₑAₖAₖA_{d}T_{d}T_{d}G_{d}T_{d}^{m}C_{d}A_{d}T_{c}^{m}C_{d}Aₖ^{m}Cₖ^{m}Cₑ | 3-9-3 | Deoxy/5′-(S)- | ekk | kke | 10 |
| | | | allyl DNA | | | |
| | | | (pos 11) | | | |
| 589422 | TₑAₖAₖA_{d}T_{g}T_{d}G_{d}T_{d}^{m}C_{d}A_{d}T_{d}^{m}C_{d}Aₖ^{m}Cₖ^{m}Cₑ | 3-9-3 | Deoxy/5′-(R)- | ekk | kke | 10 |
| | | | hydroxyethyl | | | |
| | | | DNA (pos 5) | | | |
| 589423 | TₑAₖAₖA_{d}T_{d}T_{g}G_{d}T_{d}^{m}C_{d}A_{d}T_{d}^{m}C_{d}Aₖ^{m}Cₖ^{m}Cₑ | 3-9-3 | Deoxy/5′-(R)- | ekk | kke | 10 |
| | | | hydroxyethyl | | | |
| | | | DNA (pos 6) | | | |
| 589424 | TₑAₖAₖA_{d}T_{d}T_{d}G_{d}T_{g}^{m}C_{d}A_{d}T_{d}^{m}C_{d}Aₖ^{m}Cₖ^{m}Cₑ | 3-9-3 | Deoxy/5′-(R)- | ekk | kke | 10 |
| | | | hydroxyethyl | | | |
| | | | DNA (pos 8) | | | |
| 589437 | TₑAₖAₖA_{d}T_{d}T_{d}G_{d}T_{d}^{m}C_{d}A_{d}T_{g}^{m}C_{d}Aₖ^{m}Cₖ^{m}Cₑ | 3-9-3 | Deoxy/5′-(R)- | ekk | kke | 10 |
| | | | hydroxyethyl | | | |
| | | | DNA (pos 11) | | | |
| 589426 | TₑAₖAₖA_{d}TᵢT_{d}G_{d}T_{d}^{m}C_{d}A_{d}T_{d}^{m}C_{d}Aₖ^{m}Cₖ^{m}Cₑ | 3-9-3 | Deoxy/5′-(S)- | ekk | kke | 10 |
| | | | hydroxyethyl | | | |
| | | | DNA (pos 5) | | | |
| 589427 | TₑAₖAₖA_{d}T_{d}TᵢG_{d}T_{d}^{m}C_{d}A_{d}T_{d}^{m}C_{d}Aₖ^{m}Cₖ^{m}Cₑ | 3-9-3 | Deoxy/5′-(S)- | ekk | kke | 10 |
| | | | hydroxyethyl | | | |
| | | | DNA (pos 6) | | | |
| 589428 | TₑAₖAₖA_{d}T_{d}T_{d}G_{d}Tᵢ^{m}C_{d}A_{d}T_{d}^{m}C_{d}Aₖ^{m}Cₖ^{m}C^{e} | 3-9-3 | Deoxy/5′-(S)- | ekk | kke | 10 |
| | | | hydroxyethyl | | | |
| | | | DNA (pos 8) | | | |
| 589425 | TₑAₖAₖA_{d}T_{d}T_{d}G_{d}T_{d}^{m}C_{d}A_{d}Tᵢ^{m}C_{d}Aₖ^{m}Cₖ^{m}Cₑ | 3-9-3 | Deoxy/5′-(S)- | ekk | kke | 10 |
| | | | hydroxyethyl | | | |
| | | | DNA (pos 11) | | | |

e = 2′-MOE; k = cEt

TABLE 118
Comparison of inhibition of HTT mRNA levels and selectivity of gap-
interrupted oligonucleotides with ISIS 460209 targeting HTT SNP

| ISIS | IC₅₀ (μM) | | Selectivity | Gap Chemistry | | Wing Chemistry | |
|---|---|---|---|---|---|---|---|
| NO | Mut | Wt | (wt vs. mut) | (mod position) | Motif | 5′ | 3′ |
| 460209 | 0.47 | 2.1 | 4.5 | Full deoxy | 3-9-3 | ekk | kke |
| 589414 | 1.0 | 7.6 | 7.6 | Deoxy/5′-(R)-Allyl DNA | 3-9-3 | ekk | kke |
| | | | | (pos 5) | | | |
| 589415 | 1.4 | >10 | >7.1 | Deoxy/5′-(R)-Allyl DNA | 3-9-3 | ekk | kke |
| | | | | (pos 6) | | | |
| 589416 | 2.7 | >10 | >3.7 | Deoxy/5′-(R)-Allyl DNA | 3-9-3 | ekk | kke |
| | | | | (pos 8) | | | |
| 589417 | 5.4 | >10 | >1.9 | Deoxy/5′-(R)-Allyl DNA | 3-9-3 | ekk | kke |
| | | | | (pos 11) | | | |
| 589418 | 1.2 | >10 | >8.3 | Deoxy/5′-(S)-Allyl DNA | 3-9-3 | ekk | kke |
| | | | | (pos 5) | | | |
| 589419 | 1.1 | >10 | >9.1 | Deoxy/5′-(S)-Allyl DNA | 3-9-3 | ekk | kke |
| | | | | (pos 6) | | | |
| 589420 | 3.2 | >10 | >3.1 | Deoxy/5′-(S)-Allyl DNA | 3-9-3 | ekk | kke |
| | | | | (pos 8) | | | |
| 589421 | 2.0 | >10 | >5.0 | Deoxy/5′-(S)-Allyl DNA | 3-9-3 | ekk | kke |
| | | | | (pos 11) | | | |
| 589422 | 0.73 | 3.2 | 4.4 | Deoxy/5′-(R)- | 3-9-3 | ekk | kke |
| | | | | Hydroxyethyl DNA | | | |
| | | | | (pos 5) | | | |
| 589423 | 0.92 | 9.2 | 10 | Deoxy/5′-(R)- | 3-9-3 | ekk | kke |
| | | | | Hydroxyethyl DNA | | | |
| | | | | (pos 6) | | | |
| 589424 | 0.21 | 4.4 | 21 | Deoxy/5′-(R)- | 3-9-3 | ekk | kke |
| | | | | Hydroxyethyl DNA | | | |
| | | | | (pos 8) | | | |
| 589437 | 0.73 | >10.2 | >14 | Deoxy/5′-(R)- | 3-9-3 | ekk | kke |
| | | | | Hydroxyethyl DNA | | | |
| | | | | (pos 11) | | | |
| 589426 | 0.91 | 5.1 | 5.6 | Deoxy/5′-(S)- | 3-9-3 | ekk | kke |
| | | | | Hydroxyethyl DNA | | | |
| | | | | (pos 5) | | | |
| 589427 | 0.91 | >10 | >11 | Deoxy/5′-(S)- | 3-9-3 | ekk | kke |
| | | | | Hydroxyethyl DNA | | | |
| | | | | (pos 6) | | | |
| 589428 | 1.1 | >11 | >10 | Deoxy/5′-(S)- | 3-9-3 | ekk | kke |
| | | | | Hydroxyethyl DNA | | | |
| | | | | (pos 8) | | | |
| 589425 | 1.5 | >10.5 | >7 | Deoxy/5′-(S)- | 3-9-3 | ekk | kke |
| | | | | Hydroxyethyl DNA | | | |
| | | | | (pos 11) | | | |

e = 2′-MOE; k = cEt

### Example 77

Modified Oligonucleotides Comprising 5′-(R)-Me DNA(s) Targeting Human C-Reactive Protein (hCRP)

A series of modified oligonucleotides were designed based on ISIS 353512, wherein the central gap region contains fourteen β-D-2′-deoxyribonucleoside. These modified oligonucleotides were designed by replacement of two or three β-D-2′-deoxyribonucleoside in the 14 nucleoside gap region with 5′-(R)-Me DNA(s). The thermal stability (Tₘ) and potency of these modified oligonucleotides targeting hCRP was evaluated. The 3-14-3 MOE gapmer, ISIS 353512 and 5-10-5 MOE gapmer, ISIS 330012 were included in the study for comparison.

The modified oligonucleotides and their motifs are described in Table 119. Each internucleoside linkage is a phosphorothioate (P═S) except for nucleosides followed by a subscript “o” which are phosphodiester internucleoside linkages (P═O). Nucleosides followed by a subscript “d” indicates a β-D-2′-deoxyribonucleoside. Nucleosides followed by a subscript “e” indicates a 2′-O-methoxyethyl (MOE) modified nucleoside. Nucleosides followed by a subscript “z” indicates a 5′-(R)-Me DNA. “^{m}C” indicates a 5-methyl cytosine modified nucleoside. Underlined nucleosides indicate a region comprising 5′-(R)-Me DNA modification.

### Thermal Stability Assay

The modified oligonucleotides were evaluated in thermal stability (Tₘ) assay. The Tₘ's were measured using the method described herein. A Cary 100 Bio spectrophotometer with the Cary Win UV Thermal program was used to measure absorbance vs. temperature. For the Tₘ experiments, oligonucleotides were prepared at a concentration of 8 μM in a buffer of 100 mM Na+, 10 mM phosphate, 0.1 mM EDTA, pH 7. Concentration of oligonucleotides were determined at 85° C. The oligonucleotide concentration was 4 μM with mixing of equal volumes of test oligonucleotide and complimentary RNA strand. Oligonucleotides were hybridized with the complimentary RNA strand by heating duplex to 90° C. for 5 min and allowed to cool at room temperature. Using the spectrophotometer, Tₘ measurements were taken by heating duplex solution at a rate of 0.5 C/min in cuvette starting @ 15° C. and heating to 85° C. Tₘ values were determined using Vant Hoff calculations (A₂₆₀ vs temperature curve) using non self-complementary sequences where the minimum absorbance which relates to the duplex and the maximum absorbance which relates to the non-duplex single strand are manually integrated into the program. The results are presented below.

### Cell Culture and Transfection

The modified oligonucleotides were tested in vitro. Hep3B cells were plated at a density of 40,000 cells per well and transfected using electroporation with 0.009 μM, 0.027 μM, 0.082 μM, 0.25 μM, 0.74 μM, 2.2 μM, 6.7 μM and 20 μM concentrations of antisense oligonucleotides. After a treatment period of approximately 16 hours, RNA was isolated from the cells and hCRP mRNA levels were measured by quantitative real-time PCR. Human CRP primer probe set RTS1887 was used to measure mRNA levels. hCRP mRNA levels were adjusted according to total RNA content, as measured by RIBOGREEN®.

### Analysis of IC₅₀'s

The half maximal inhibitory concentration (IC₅₀) of each oligonucleotide is presented below and was calculated by plotting the concentrations of oligonucleotides used versus the percent inhibition of hCRP mRNA expression achieved at each concentration, and noting the concentration of oligonucleotide at which 50% inhibition of hCRP mRNA expression was achieved compared to the control.

As illustrated in Table 120, treatment with the newly designed oligonucleotides showed no improvement in potency as compared to the controls, ISIS 353512 and 330012.

TABLE 119
Gap-interrupted oligonucleotides comprising 5′-(R)-Me DNA
targeting hCRP

| | | | | Wing | | | SEQ |
|---|---|---|---|---|---|---|---|
| ISIS | | | Gap | Chemistry | | Linkage | ID |
| NO | Sequence (5′ to 3′) | Motif | Chemistry | 5′ | 3′ | backbone | NO |
| 353512 | Tₑ^{m}Cₑ^{m}Cₑ^{m}C_{d}A_{d}T_{d}T_{d}T_{d}^{m}C_{d}A_{d} | 3-14-3 | Full deoxy | eee | eee | Full PS | 83 |
| | G_{d}G_{d}A_{d}G_{d}A_{d}^{m}C_{d}^{m}C_{d}TₑGₑGₑ | | | | | | |
| 546127 | Tₑ^{m}Cₑ^{m}Cₑ^{m}C_{d}A_{d}T_{d}T_{d}T_{d}^{m}C_{do}A_{zo} | 3-14-3 | Deoxy/5′-(R)- | eee | eee | Mixed | 83 |
| | G_{z}G_{d}A_{d}G_{d}A_{d}^{m}C_{d}^{m}C_{d}TₑGₑGₑ | | Me DNA | | | PS/PO | |
| 544810 | Tₑ^{m}Cₑ^{m}Cₑ^{m}C_{d}A_{d}T_{d}T_{d}T_{d}^{m}C_{d}A_{d} | 3-14-3 | Deoxy/5′-(R)- | eee | eee | Mixed | 83 |
| | G_{d}G_{d}A_{d}G_{d}A_{do}^{m}C_{zo}^{m}C_{z}TₑGₑGₑ | | Me DNA | | | PS/PO | |
| 544806 | Tₑ^{m}Cₑ^{m}Cₑₒ^{m}C_{zo}A_{zo}T_{z}T_{d}T_{d}^{m}C_{d}A_{d} | 3-14-3 | Deoxy/5′-(R)- | eee | eee | Mixed | 83 |
| | G_{d}G_{d}A_{d}G_{d}A_{d}^{m}C_{d}^{m}C_{d}TₑGₑGₑ | | Me DNA | | | PS/PO | |
| 544807 | Tₑ^{m}Cₑ^{m}Cₑ^{m}C_{d}A_{d}T_{do}T_{zo}T_{zo}^{m}C_{z}A_{d} | 3-14-3 | Deoxy/5′-(R)- | eee | eee | Mixed | 83 |
| | G_{d}G_{d}A_{d}G_{d}A_{d}^{m}C_{d}^{m}C_{d}TₑGₑGₑ | | Me DNA | | | PS/PO | |
| 544809 | Tₑ^{m}Cₑ^{m}Cₑ^{m}C_{d}A_{d}T_{d}T_{d}T_{d}^{m}C_{d}A_{d} | 3-14-3 | Deoxy/5′-(R)- | eee | eee | Mixed | 83 |
| | G_{d}G_{do}A_{zo}G_{zo}A_{z}^{m}C_{d}^{m}C_{d}TₑGₑGₑ | | Me DNA | | | PS/PO | |
| 330012 | Tₑ^{m}Cₑ^{m}Cₑ^{m}CₑAₑT_{d}T_{d}T_{d}^{m}C_{d}A_{d} | 5-10-5 | Full deoxy | e5 | e5 | Full PS | 83 |
| | G_{d}G_{d}A_{d}G_{d}A_{d}^{m}Cₑ^{m}CₑTₑGₑGₑ | | | | | | |

e = 2′-MOE (e.g. e5 = eeeee)

TABLE 120
Effect of gap-interrupted oligonucleotide
treatment on Tm and hCRP inhibition

| | | | | | Wing | | |
|---|---|---|---|---|---|---|---|
| ISIS | Tm | IC₅₀ | | Gap | Chemistry | | Linkage |
| NO | (° C.) | (μM) | Motif | Chemistry | 5′ | 3′ | backbone |
| 353512 | 66.7 | 1.1 | 3-14-3 | Full deoxy | eee | eee | Full PS |
| 546127 | 65.9 | 2.5 | 3-14-3 | Deoxy/5′-(R)- | eee | eee | Mixed |
| | | | | Me DNA | | | PS/PO |
| 544810 | 64.3 | 2.4 | 3-14-3 | Deoxy/5′-(R)- | eee | eee | Mixed |
| | | | | Me DNA | | | PS/PO |
| 544806 | 62.8 | 2.8 | 3-14-3 | Deoxy/5′-(R)- | eee | eee | Mixed |
| | | | | Me DNA | | | PS/PO |
| 544807 | 65.1 | 2.7 | 3-14-3 | Deoxy/5′-(R)- | eee | eee | Mixed |
| | | | | Me DNA | | | PS/PO |
| 544809 | 64.2 | 5.0 | 3-14-3 | Deoxy/5′-(R)- | eee | eee | Mixed |
| | | | | Me DNA | | | PS/PO |
| 330012 | 71.7 | 0.6 | 5-10-5 | Full deoxy | e5 | e5 | Full PS |

e = 2′-MOE (e.g. e5 = eeeee), PS/PO = phosphorothioate/phosphodiester internucleoside linkage

### Example 78

Human Peripheral Blood Mononuclear Cells (hPBMC) Assay Protocol—In Vitro

The hPBMC assay was performed using BD Vautainer CPT tube method. A sample of whole blood from volunteered donors with informed consent at US HealthWorks clinic (Faraday & El Camino Real, Carlsbad) was obtained and collected in 4-15 BD Vacutainer CPT 8 ml tubes (VWR Cat. #BD362753). The approximate starting total whole blood volume in the CPT tubes for each donor was recorded using the PBMC assay data sheet.

The blood sample was remixed immediately prior to centrifugation by gently inverting tubes 8-10 times. CPT tubes were centrifuged at rt (18-25° C.) in a horizontal (swing-out) rotor for 30 min. at 1500-1800 RCF with brake off (2700 RPM Beckman Allegra 6R). The cells were retrieved from the buffy coat interface (between Ficoll and polymer gel layers); transferred to a sterile 50 ml conical tube and pooled up to 5 CPT tubes/50 ml conical tube/donor. The cells were then washed twice with PBS (Ca⁺⁺, Mg⁺⁺ free; GIBCO). The tubes were topped up to 50 ml and mixed by inverting several times. The sample was then centrifuged at 330×g for 15 minutes at rt (1215 RPM in Beckman Allegra 6R) and aspirated as much supernatant as possible without disturbing pellet. The cell pellet was dislodged by gently swirling tube and resuspended cells in RPMI+10% FBS+pen/strep (˜1 ml/10 ml starting whole blood volume). A 60 μl sample was pipette into a sample vial (Beckman Coulter) with 600 μl VersaLyse reagent (Beckman Coulter Cat #A09777) and was gently vortexed for 10-15 sec. The sample was allowed to incubate for 10 min. at rt and being mixed again before counting. The cell suspension was counted on Vicell XR cell viability analyzer (Beckman Coulter) using PBMC cell type (dilution factor of 1:11 was stored with other parameters). The live cell/ml and viability were recorded. The cell suspension was diluted to 1×10⁷ live PBMC/ml in RPMI+10% FBS+pen/strep.

The cells were plated at 5×10⁵ in 50 μl/well of 96-well tissue culture plate (Falcon Microtest). 50 μl/well of 2× concentration oligos/controls diluted in RPMI+10% FBS+pen/strep. was added according to experiment template (100 μl/well total). Plates were placed on the shaker and allowed to mix for approx. 1 min. After being incubated for 24 hrs at 37° C.; 5% CO₂, the plates were centrifuged at 400×g for 10 minutes before removing the supernatant for MSD cytokine assay (i.e. human IL-6, IL-10, IL-8 and MCP-1).

### Example 79

Evaluation of the Proinflammatory Effects in hPBMC Assay for 5′-(R)-Me DNA Containing Modified Oligonucleotides—In Vitro Study

The modified oligonucleotides targeting hCRP from Example 77 were tested and evaluated for the proinflammatory response in hPBMC assay using methods described previously in Example 78. The hPBMCs were isolated from fresh, volunteered donors and were treated with modified oligonucleotides at 0, 0.0128, 0.064, 0.32, 1.6, 8, 40 and 200 μM concentrations using the hPBMC assay protocol described herein. After a 24 hr treatment, the cytokine levels were measured.

IL-6 was used as the primary readout. The resulting IL-6 level was compared to the positive control, ISIS 353512 and negative control, ISIS 104838. The results are presented in Table 121. As illustrated, reduction in proinflammatory response was achieved with the newly designed oligonucleotides at doses evaluated as compared to the positive control, ISIS 353512.

ISIS 104838 designated herein as SEQ ID NO: 84, is a 5-10-5 MOE gapmer with the following sequence, Gₑ^{m}CₑTₑGₑAₑT_{d}T_{d}A_{d}G_{d}A_{d}G_{d}A_{d}G_{d}A_{d}G_{d}GₑTₑ^{m}Cₑ^{m}Cₑ^{m}Cₑ. Each internucleoside linkage is a phosphorothioate (P═S). Each nucleoside followed by a subscript “d” is a β-D-2′-deoxyribonucleoside. Each “^{m}C” is a 5-methyl cytosine modified nucleoside and each nucleoside followed by a subscript “e” is a 2′-O-methoxyethyl (MOE) modified nucleoside.

TABLE 121
Effect of gap-interrupted oligonucleotide treatment
on proinflammatory response in hPBMC

| | | | | | Wing | | |
|---|---|---|---|---|---|---|---|
| ISIS | Conc. | IL-6 | | Gap | Chemistry | | Linkage |
| NO | (uM) | (pg/mL) | Motif | Chemistry | 5′ | 3′ | backbone |
| 353512 | 0 | 26.9 | 3-14-3 | Full deoxy | eee | eee | Full PS |
| (pos | 0.0128 | 10.6 | | | | | |
| control) | 0.064 | 73.3 | | | | | |
| | 0.32 | 219.8 | | | | | |
| | 1.6 | 200.1 | | | | | |
| | 8 | 287.8 | | | | | |
| | 40 | 376.9 | | | | | |
| | 200 | 181.5 | | | | | |
| 546127 | 0 | 11.5 | 3-14-3 | Deoxy/5′-(R)- | eee | eee | Mixed |
| | 0.0128 | 15.1 | | Me DNA | | | PS/PO |
| | 0.064 | 19.0 | | | | | |
| | 0.32 | 37.3 | | | | | |
| | 1.6 | 67.5 | | | | | |
| | 8 | 86.3 | | | | | |
| | 40 | 111.2 | | | | | |
| | 200 | 83.1 | | | | | |
| 544810 | 0 | 11.5 | 3-14-3 | Deoxy/5′-(R)- | eee | eee | Mixed |
| | 0.0128 | 13.9 | | Me DNA | | | PS/PO |
| | 0.064 | 15.1 | | | | | |
| | 0.32 | 24.9 | | | | | |
| | 1.6 | 34.0 | | | | | |
| | 8 | 66.2 | | | | | |
| | 40 | 96.8 | | | | | |
| | 200 | 76.5 | | | | | |
| 06/544806 | 0 | 11.3 | 3-14-3 | Deoxy/5′-(R)- | eee | eee | Mixed |
| | 0.0128 | 10.8 | | Me DNA | | | PS/PO |
| | 0.064 | 25.8 | | | | | |
| | 0.32 | 15.6 | | | | | |
| | 1.6 | 25.4 | | | | | |
| | 8 | 52.3 | | | | | |
| | 40 | 69.3 | | | | | |
| | 200 | 341.7 | | | | | |
| 06/544807 | 0 | 13.3 | 3-14-3 | Deoxy/5′-(R)- | eee | eee | Mixed |
| | 0.0128 | 13.7 | | Me DNA | | | PS/PO |
| | 0.064 | 18.4 | | | | | |
| | 0.32 | 53.3 | | | | | |
| | 1.6 | 18.4 | | | | | |
| | 8 | 164.9 | | | | | |
| | 40 | 202.7 | | | | | |
| | 200 | 606.5 | | | | | |
| 06/544809 | 0 | 10.8 | 3-14-3 | Deoxy/5′-(R)- | eee | eee | Mixed |
| | 0.0128 | 13.3 | | Me DNA | | | PS/PO |
| | 0.064 | 14.3 | | | | | |
| | 0.32 | 34.8 | | | | | |
| | 1.6 | 62.3 | | | | | |
| | 8 | 100.9 | | | | | |
| | 40 | 213.1 | | | | | |
| | 200 | 225.0 | | | | | |
| 06/330012 | 0 | 10.9 | 5-10-5 | Full deoxy | e5 | e5 | Full PS |
| | 0.0128 | 12.9 | | | | | |
| | 0.064 | 10.8 | | | | | |
| | 0.32 | 25.3 | | | | | |
| | 1.6 | 44.2 | | | | | |
| | 8 | 87.5 | | | | | |
| | 40 | 80.2 | | | | | |
| | 200 | 82.3 | | | | | |
| 07/104838 | 0 | 9.3 | 5-10-5 | Full deoxy | e5 | e5 | Full PS |
| (neg | 0.0128 | 10.4 | | | | | |
| control) | 0.064 | 17.6 | | | | | |
| | 0.32 | 30.1 | | | | | |
| | 1.6 | 53.9 | | | | | |
| | 8 | 124.8 | | | | | |
| | 40 | 94.5 | | | | | |
| | 200 | 89.3 | | | | | |

e = 2′-MOE (e.g. e5 = eeeee)

### Example 80

Evaluation of the Proinflammatory Effects in hPBMC Assay for a Modified Oligonucleotide Comprising Methyl Thiophosphonate Internucleoside Linkages—In Vitro Study

A modified oligonucleotide was designed based on the 3/14/3 MOE gapmer, ISIS 353512. This modified oligonucleotide was created by having alternating methyl thiophosphonate (—P(CH₃)(═S)—) internucleoside linkages throughout the gap region. The proinflammatory effect of the modified oligonucleotide targeting hCRP was evaluated in hPBMC assay using the protocol described in Example 78.

The modified oligonucleotide and its motif are described in Table 122. Each internucleoside linkage is a phosphorothioate (P═S) except for nucleosides followed by a subscript “w”. Each nucleoside followed by a subscript “w” indicates a methyl thiophosphonate internucleoside linkage (—P(CH₃)(═S)—). Nucleosides followed by a subscript “d” is a β-D-2′-deoxyribonucleoside. Nucleosides followed by a subscript “e” indicates a 2′-O-methoxyethyl (MOE) modified nucleoside. “^{m}C” indicates a 5-methyl cytosine modified nucleoside.

The hPBMCs were isolated from fresh, volunteered donors and were treated with modified oligonucleotides at 0, 0.0128, 0.064, 0.32, 1.6, 8, 40 and 200 μM concentrations. After a 24 hr treatment, the cytokine levels were measured.

IL-6 was used as the primary readout. The resulting IL-6 level was compared to the positive control oligonucleotide, ISIS 353512 and negative control, ISIS 104838. The results from two donors denoted as “Donor 1” and “Donor 2” are presented in Table 123. As illustrated, reduction in proinflammatory response was achieved with the newly designed oligonucleotide at doses evaluated as compared to the positive control, ISIS 353512.

TABLE 122
Modified oligonucleotide comprising alternating methyl
thiophosphonate internucleoside linkages throughout
the gap region

| | | | | Wing | | SEQ |
|---|---|---|---|---|---|---|
| ISIS | | | Gap | Chemistry | | ID |
| NO | Sequence (5′ to 3′) | Motif | Chemistry | 5′ | 3′ | NO |
| 353512 | Tₑ^{m}Cₑ^{m}Cₑ^{m}C_{d}A_{d}T_{d}T_{d}T_{d}^{m}C_{d}A_{d}G_{d} | 3-14-3 | Full deoxy | eee | eee | 83 |
| | G_{d}A_{d}G_{d}A_{d}^{m}C_{d}^{m}C_{d}TₑGₑGₑ | | | | | |
| 560221 | Tₑ^{m}Cₑ^{m}CₑC_{dw}A_{d}T_{dw}T_{d}T_{dw}^{m}C_{d}A_{dw}G_{d}G_{dw} | 3-14-3 | Deoxy/methyl | eee | eee | 83 |
| | A_{d}G_{dw}A_{d}C_{dw}^{m}C_{d}TₑGₑGₑ | | thiophosphonate | | | |
| 104838 | Gₑ^{m}CₑTₑGₑAₑT_{d}T_{d}A_{d}G_{d}A_{d}G_{d}A_{d} | 5-10-5 | Full deoxy | e5 | e5 | 84 |
| | G_{d}A_{d}G_{d}GₑTₑ^{m}Cₑ^{m}Cₑ^{m}Cₑ | | | | | |

e = 2′-MOE (e.g. e5 = eeeee)

TABLE 123
Effect of modified oligonucleotide treatment
on proinflammatory response in hPBMC assay

| | | | | | | Wing | |
|---|---|---|---|---|---|---|---|
| ISIS | Conc. | IL-6 (Donor 1) | IL-6 (Donor 2) | | Gap | Chemistry | |
| NO | (μM) | (pg/mL) | (pg/mL) | Motif | Chemistry | 5′ | 3′ |
| 353512 | 0 | 6.3 | 7.8 | 3-14-3 | Full deoxy | eee | eee |
| | 0.0128 | 8.3 | 10.2 | | | | |
| | 0.064 | 77.2 | 118.2 | | | | |
| | 0.32 | 151.9 | 394.3 | | | | |
| | 1.6 | 152.4 | 395.3 | | | | |
| | 8 | 147.6 | 337.2 | | | | |
| | 40 | 122.5 | 228.4 | | | | |
| | 200 | 119.7 | 193.5 | | | | |
| 560221 | 0 | 5.6 | 7.6 | 3-14-3 | Deoxy/methyl | eee | eee |
| | 0.0128 | 6.4 | 6.9 | | thiophosphonate | | |
| | 0.064 | 6.7 | 7.6 | | | | |
| | 0.32 | 7.6 | 8.9 | | | | |
| | 1.6 | 9.1 | 11.8 | | | | |
| | 8 | 17.5 | 24.3 | | | | |
| | 40 | 65.8 | 50.2 | | | | |
| | 200 | 60.0 | 100.4 | | | | |
| 104838 | 0 | 5.8 | 7.3 | 5-10-5 | Full deoxy | e5 | e5 |
| | 0.0128 | 7.7 | 7.9 | | | | |
| | 0.064 | 7.5 | 11.6 | | | | |
| | 0.32 | 15.1 | 22.0 | | | | |
| | 1.6 | 73.1 | 112.8 | | | | |
| | 8 | 29.6 | 51.5 | | | | |
| | 40 | 41.6 | 69.5 | | | | |
| | 200 | 55.4 | 4018 | | | | |

e = 2′-MOE (e.g. e5 = eeeee)

### Example 81

### Modified Oligonucleotides Comprising Methyl Phosphonate Internucleoside Linkage Targeting HTT SNP—In Vitro Study

ISIS 558255 and 558256 from Example 49 were selected and evaluated for their effect on mutant and wild type HTT mRNA expression levels targeting rs7685686. ISIS 46020 was included in the study for comparison. The position on the oligonucleotides opposite to the SNP position, as counted from the 5′-terminus is position 8.

Heterozygous fibroblast GM04022 cell line was used for the in vitro assay (from Coriell Institute). Cultured GM04022 cells at a density of 25,000 cells per well were transfected using electroporation with 0.12, 0.37, 1.1, 3.3 and 10 μM concentrations of modified oligonucleotides. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 μL 2×PCR buffer, 101 μL primers (300 μM from ABI), 1000 μL water and 40.4 μL RT MIX. To each well was added 15 μL of this mixture and 5 μL of purified RNA. The mutant and wild-type HTT mRNA levels were measured simultaneously by using two different fluorophores, FAM for mutant allele and VIC for wild-type allele. The HTT mRNA levels were adjusted according to total RNA content, as measured by RIBOGREEN.

The IC₅₀s and selectivities as expressed in “fold” were measured and calculated using methods described previously in Example 41. As illustrated in Table 124, improvement in selectivity and potency was achieved with modified oligonucleotides comprising methyl phosphonate internucleoside linkage as compared to ISIS 460209.

TABLE 124
Comparison of selectivity in inhition of HTT mRNA levels of antisense
oligonucleotides with ISIS 460209 targeted to rs7685686 in GM4022 cells

| ISIS | IC₅₀ (μM) | | Selectivity | | | Wing Chemistry | | SEQ |
|---|---|---|---|---|---|---|---|---|
| NO | Mut | Wt | (wt vs mut) | Motif | Gap Chemistry | 5′ | 3′ | ID NO |
| 460209 | 0.30 | 0.99 | 3.3 | 3-9-3 | Full deoxy | ekk | kke | 10 |
| 558255 | 0.19 | 1.3 | 6.8 | 3-9-3 | Deoxy/Methyl | ekk | kke | 10 |
| | | | | | phosphonate | | | |
| 558256 | 0.20 | 1.3 | 6.5 | 3-9-3 | Deoxy/Methyl | ekk | kke | 10 |
| | | | | | phosphonate | | | |

e = 2′-MOE (e.g. e5 = eeeee), k = cEt

### Example 82

### Modified Oligonucleotides Comprising Methyl Phosphonate or Phosphonoacetate Internucleoside Linkage(s) Targeting HTT SNP

A series of modified oligonucleotides were designed based on ISIS 460209 wherein the gap region contains nine β-D-2′-deoxyribonucleosides. The modified oligonucleotides were synthesized to include one or more methyl phosphonate or phosphonoacetate internucleoside linkage modifications within the gap region. The oligonucleotides with modified phosphorus containing backbone were tested for their ability to selectively inhibit mutant (mut) HTT mRNA expression levels targeting rs7685686 while leaving the expression of the wild-type (wt) intact. The potency and selectivity of the modified oligonucleotides were evaluated and compared to ISIS 460209.

The position on the oligonucleotides opposite to the SNP position, as counted from the 5′-terminus is position 8.

The modified oligonucleotides and their motifs are described in Table 125. Each internucleoside linkage is a phosphorothioate (P═S) except for the internucleoside linkage having a subscript “x” or “y”. Each nucleoside followed by a subscript “x” indicates a methyl phosphonate internucleoside linkage (—P(CH₃)(═O)—). Each nucleoside followed by a subscript “y” indicates a phosphonoacetate internucleoside linkage (—P(CH₂CO₂)(═O)—). Nucleosides followed by a subscript “d” is a β-D-2′-deoxyribonucleoside. Nucleosides followed by a subscript “e” indicates a 2′-O-methoxyethyl (MOE) modified nucleoside. Nucleosides followed by a subscript “k” indicates a 6′-(S)—CH₃ bicyclic nucleoside (e.g. cEt). “^{m}C” indicates a 5-methyl cytosine modified nucleoside.

The modified oligonucleotides were tested in vitro. Heterozygous fibroblast GM04022 cell line was used (from Coriell Institute). Cultured GM04022 cells at a density of 25,000 cells per well were transfected using electroporation with 0.12, 0.37, 1.1, 3.3 and 10 μM concentrations of modified oligonucleotides. After a treatment period of approximately 24 hours, cells were washed with DPBS buffer and lysed. RNA was extracted using Qiagen RNeasy purification and mRNA levels were measured by quantitative real-time PCR using ABI assay C_2229297_10 which measures at dbSNP rs362303. RT-PCR method in short; A mixture was made using 2020 GL 2×PCR buffer, 101 μL primers (300 μM from ABI), 1000 uL water and 40.4 μL RT MIX. To each well was added 15 μL of this mixture and 5 μL of purified RNA. The mutant and wild-type HTT mRNA levels were measured simultaneously by using two different fluorophores, FAM for mutant allele and VIC for wild-type allele. The HTT mRNA levels were adjusted according to total RNA content, as measured by RIBOGREEN.

The IC₅₀s and selectivities as expressed in “fold” were measured and calculated using methods described previously in Example 41. As illustrated in Table 126, most of the newly design oligonucleotides achieved improvement in selectivity while maintaining potency as compared to ISIS 460209.

TABLE 125
Modified oligonucleotides comprising methyl phosphonate or
phosphonoacetate internucleoside linkage(s) targeting HTT SNP

| | | | | Wing | | SEQ |
|---|---|---|---|---|---|---|
| ISIS | | | | Chemistry | | ID |
| NO | Sequence (5′ to 3′) | Motif | Gap Chemistry | 5′ | 3′ | NO |
| 460209 | TₑAₖAₖA_{d}T_{d}T_{d}G_{d}T_{d}^{m}C_{d}A_{d}T_{d}^{m}C_{d}Aₖ^{m}Cₖ^{m}Cₑ | 3-9-3 | Full deoxy | ekk | kke | 10 |
| 566276 | TₑAₖAₖA_{d}T_{d}T_{d}G_{dx}T_{d}^{m}C_{d}A_{d}T_{d}^{m}C_{d}Aₖ^{m}Cₖ^{m}Cₑ | 3-9-3 | Deoxy/Methyl | ekk | kke | 10 |
| | | | phosphonate | | | |
| 566277 | TₑAₖAₖA_{d}T_{d}T_{d}G_{d}T_{dx}^{m}C_{d}A_{d}T_{d}^{m}C_{d}Aₖ^{m}Cₖ^{m}Cₑ | 3-9-3 | Deoxy/Methyl | ekk | kke | 10 |
| | | | phosphonate | | | |
| 566278 | TₑAₖAₖA_{d}T_{d}T_{d}G_{d}T_{d}^{m}C_{dx}A_{d}T_{d}^{m}C_{d}Aₖ^{m}Cₖ^{m}Cₑ | 3-9-3 | Deoxy/Methyl | ekk | kke | 10 |
| | | | phosphonate | | | |
| 566279 | TₑAₖAₖA_{d}T_{d}T_{d}G_{d}T_{d}^{m}C_{d}A_{dx}T_{d}^{m}C_{d}Aₖ^{m}Cₖ^{m}Cₑ | 3-9-3 | Deoxy/Methyl | ekk | kke | 10 |
| | | | phosphonate | | | |
| 566280 | TₑAₖAₖA_{d}T_{d}T_{d}G_{d}T_{d}^{m}C_{d}A_{d}T_{dx}^{m}C_{d}Aₖ^{m}Cₖ^{m}Cₑ | 3-9-3 | Deoxy/Methyl | ekk | kke | 10 |
| | | | phosphonate | | | |
| 566283 | TₑAₖAₖA_{d}T_{dx}T_{dx}G_{d}T_{d}^{m}C_{d}A_{d}T_{d}^{m}C_{d}Aₖ^{m}Cₖ^{m}Cₑ | 3-9-3 | Deoxy/Methyl | ekk | kke | 10 |
| | | | phosphonate | | | |
| 573815 | TₑAₖAₖA_{d}T_{dy}T_{d}G_{d}T_{d}^{m}C_{d}A_{d}T_{d}^{m}C_{d}Aₖ^{m}Cₖ^{m}Cₑ | 3-9-3 | Deoxy/ | ekk | kke | 10 |
| | | | Phosphonoacetate | | | |
| 573816 | TₑAₖAₖA_{d}T_{d}T_{dy}G_{d}T_{d}^{m}C_{d}A_{d}T_{d}^{m}C_{d}Aₖ^{m}Cₖ^{m}Cₑ | 3-9-3 | Deoxy/ | ekk | kke | 10 |
| | | | Phosphonoacetate | | | |
| 573817 | TₑAₖAₖA_{d}T_{d}T_{d}G_{d}T_{dy}^{m}C_{d}A_{d}T_{d}^{m}C_{d}Aₖ^{m}Cₖ^{m}Cₑ | 3-9-3 | Deoxy/ | ekk | kke | 10 |
| | | | Phosphonoacetate | | | |
| 573818 | TₑAₖAₖA_{d}T_{d}T_{d}G_{d}T_{d}^{m}C_{d}A_{d}T_{dy}^{m}C_{d}Aₖ^{m}Cₖ^{m}Cₑ | 3-9-3 | Deoxy/ | ekk | kke | 10 |
| | | | Phosphonoacetate | | | |

e = 2′-MOE, k = cEt

TABLE 126
Comparison of selectivity in inhition of HTT mRNA levels of antisense
oligonucleotides with ISIS 460209 targeted to rs7685686 in GM4022 cells

| ISIS | Mut IC₅₀ | Selectivity | | | Wing Chemistry | | SEQ |
|---|---|---|---|---|---|---|---|
| NO | (μM)) | (wt vs mut) | Motif | Gap Chemistry | 5′ | 3′ | ID NO |
| 460209 | 0.15 | 9.4 | 3-9-3 | Full deoxy | ekk | kke | 10 |
| 566276 | 0.76 | 12.8 | 3-9-3 | Deoxy/Methyl phosphonate | ekk | kke | 10 |
| 566277 | 0.20 | 17 | 3-9-3 | Deoxy/Methyl phosphonate | ekk | kke | 10 |
| 566278 | 0.25 | 8.9 | 3-9-3 | Deoxy/Methyl phosphonate | ekk | kke | 10 |
| 566279 | 0.38 | — | 3-9-3 | Deoxy/Methyl phosphonate | ekk | kke | 10 |
| 566280 | 0.27 | 47 | 3-9-3 | Deoxy/Methyl phosphonate | ekk | kke | 10 |
| 566283 | 0.8 | >100 | 3-9-3 | Deoxy/Methyl phosphonate | ekk | kke | 10 |
| 573815 | 0.16 | 18.8 | 3-9-3 | Deoxy/Phosphonoacetate | ekk | kke | 10 |
| 573816 | 0.55 | 18.1 | 3-9-3 | Deoxy/Phosphonoacetate | ekk | kke | 10 |
| 573817 | 0.17 | 22.5 | 3-9-3 | Deoxy/Phosphonoacetate | ekk | kke | 10 |
| 573818 | 0.24 | 13.5 | 3-9-3 | Deoxy/Phosphonoacetate | ekk | kke | 10 |

e = 2′-MOE, k = cEt

### Example 83

### Modified Oligonucleotides Comprising Methyl Phosphonate Internucleoside Linkages Targeting PTEN and SRB-1—In Vivo Study

Additional modified oligonucleotides were designed based on ISIS 482050 and 449093 wherein the gap region contains ten β-D-2′-deoxyribonucleosides. The modified oligonucleotides were designed by introducing two methyl phosphonate internucleoside linkages at the 5′-end of the gap region with a 3/10/3 motif. The oligonucleotides were evaluated for reduction in PTEN and SRB-1 mRNA expression levels in vivo. The parent gapmers, ISIS 482050 and 449093 were included in the study for comparison.

The modified oligonucleotides and their motifs are described in Table 127. Each internucleoside linkage is a phosphorothioate (P═S) except for the internucleoside linkage having a subscript “x”. Each nucleoside followed by a subscript “x” indicates a methyl phosphonate internucleoside linkage (—P(CH₃)(═O)—). Nucleosides followed by a subscript “d” is a β-D-2′-deoxyribonucleoside. Nucleosides followed by a subscript “k” indicates a 6′-(S)—CH₃ bicyclic nucleoside (e.g. cEt). “^{m}C” indicates a 5-methyl cytosine modified nucleoside.

### Treatment

Six week old BALB/C mice (purchased from Charles River) were injected subcutaneously twice a week for three weeks at dosage 10 mg/kg or 20 mg/kg with the modified oligonucleotides shown below or with saline control. Each treatment group consisted of 3 animals. The mice were sacrificed 48 hrs following last administration, and organs and plasma were harvested for further analysis.

mRNA Analysis

Liver tissues were homogenized and mRNA levels were quantitated using real-time PCR and normalized to RIBOGREEN as described herein. The results in Table 128 are listed as PTEN or SRB-1 mRNA expression for each treatment group relative to saline-treated control (% UTC). As illustrated, reduction in PTEN or SRB-1 mRNA expression levels was achieved with the oligonucleotides comprising two methyl phosphonate internucleoside linkages at the 5′-end of the gap region, ISIS 582073 and 582074.

### Plasma Chemistry Markers

Plasma chemistry markers such as liver transaminase levels, alanine aminotranferase (ALT) in serum were measured relative to saline injected mice and the results are presented in Table 128. Treatment with the oligonucleotides resulted in reduction in ALT level compared to treatment with the parent gapmer, ISIS 482050 or 449093. The results suggest that introduction of methyl phosphonate internucleoside linkage(s) can be useful for reduction of hepatoxicity profile of otherwise unmodified parent gapmers.

### Body and Organ Weights

Body weights, as well as liver, kidney and spleen weights were measured at the end of the study. The results below are presented as the average percent of body and organ weights for each treatment group relative to saline-treated control. As illustrated in Table 129, treatment with ISIS 582073 resulted in a reduction in liver and spleen weights compared to treatment with the parent gapmer, ISIS 482050. The remaining oligonucleotide, ISIS 582074 did not cause any changes in body and organ weights outside the expected range as compared to ISIS 449093.

TABLE 127
Modified oligonucleotides comprising methyl phosphonate internu-
cleoside linkages

| | | | | Wing | | SEQ |
|---|---|---|---|---|---|---|
| ISIS | | | Gap | Chemistry | | ID |
| NO | Sequence (5′ to 3′) | Motif | Chemistry | 5′ | 3′ | NO. |
| 482050 | AₖTₖ^{m}CₖA_{d}T_{d}G_{d}G_{d}^{m}C_{d}T_{d}G_{d}^{m}C_{d}A_{d}G_{d}^{m}CₖTₖTₖ | 3-10-3 | Full deoxy | kkk | kkk | 85 |
| 582073 | AₖTₖ^{m}CₖA_{dx}T_{dx}G_{d}G_{d}^{m}C_{d}T_{d}G_{d}^{m}C_{d}A_{d}G_{d}^{m}CₖTₖTₖ | 3-10-3 | Deoxy/Methyl | kkk | kkk | 85 |
| | | | phosphonate | | | |
| 449093 | TₖTₖ^{m}CₖA_{d}G_{d}T_{d}^{m}C_{d}A_{d}T_{d}G_{d}A_{d}^{m}C_{d}T_{d}Tₖ^{m}Cₖ^{m}Cₖ | 3-10-3 | Full deoxy | kkk | kkk | 86 |
| 582074 | TₖTₖ^{m}CₖA_{dx}G_{dx}T_{d}^{m}C_{d}A_{d}T_{d}G_{d}A_{d}^{m}C_{d}T_{d}Tₖ^{m}Cₖ^{m}Cₖ | 3-10-3 | Deoxy/Methyl | kkk | kkk | 86 |
| | | | phosphonate | | | |

k = cEt

TABLE 128
Effect of modified oligonucleotide treatment on target
reduction and liver function in BALB/C mice

| ISIS | | Dosage | % | ALT | | Gap | Wing Chemistry | | SEQ |
|---|---|---|---|---|---|---|---|---|---|
| NO. | Target | (mg/kg/wk) | UTC | (IU/L) | Motif | Chemistry | 5′ | 3′ | ID NO. |
| Saline | — | 0 | 100 | 30 | — | — | — | — | — |
| 482050 | PTEN | 10 | 50 | 228 | 3-10-3 | Full deoxy | kkk | kkk | 85 |
| 482050 | | 20 | 36.1 | 505 | | | | | |
| 582073 | | 10 | 72.2 | 47.7 | | Deoxy/Methyl | kkk | kkk | 85 |
| 582073 | | 20 | 57.4 | 46 | | phosphonate | | | |
| 449093 | SRB-1 | 10 | 48 | 543 | 3-10-3 | Full deoxy | kkk | kkk | 86 |
| 449093 | | 20 | 18.5 | 1090 | | | | | |
| 582074 | | 10 | 51.3 | 58.3 | | Deoxy/Methyl | kkk | kkk | 86 |
| 582074 | | 20 | 30.3 | 126.3 | | phosphonate | | | |

k = cEt

TABLE 129
Effect of modified oligonucleotide treatment on body and organ weights in BALB/C mice

| ISIS | | Dosage | Body wt rel to | Liver/Body | Spleen/Body | Kidney/Body | SEQ |
|---|---|---|---|---|---|---|---|
| NO. | Target | (mg/kg/wk) | predose (%) | Wt (%) | Wt (%) | Wt (%) | ID NO. |
| Saline | — | 0 | 108.4 | 100 | 100 | 100 | |
| 482050 | PTEN | 10 | 107.4 | 154.9 | 141.8 | 115.7 | 85 |
| 482050 | | 20 | 111.3 | 176.7 | 142.3 | 112.5 | |
| 582073 | | 10 | 108.9 | 122.9 | 111.7 | 100.0 | 85 |
| 582073 | | 20 | 107.9 | 133.8 | 114.6 | 102.9 | |
| 449093 | SRB-1 | 10 | 101.3 | 105.9 | 117.9 | 89.3 | 86 |
| 449093 | | 20 | 95.3 | 118.6 | 129.6 | 93.0 | |
| 582074 | | 10 | 107.1 | 92.2 | 116.4 | 89.2 | 86 |
| 582074 | | 20 | 103.8 | 95.5 | 128.8 | 91.9 | |

### Example 84

### Modified Oligonucleotides Comprising Methyl Phosphonate Internucleoside Linkages Targeting Target-Y—In Vivo Study

Additional modified oligonucleotides were designed in the same manner as the antisense oligonucleotides described in Example 24, wherein two methyl phosphonate internucleoside linkages are introduced at the 5′-end of the gap region. The modified oligonucleotides were designed based on ISIS 464917, 465178, 465984 and 466456 with a 3/10/3 motif. The oligonucleotides were evaluated for reduction in Target-Y mRNA expression levels in vivo. The parent gapmers, ISIS 464917, 465178, 465984 and 466456 were included in the study for comparison.

The modified oligonucleotides and their motifs are presented in Table 130. Each internucleoside linkage is a phosphorothioate (P═S) except for the internucleoside linkage having a subscript “x”. Each nucleoside followed by a subscript “x” indicates a methyl phosphonate internucleoside linkage (—P(CH₃)(═O)—). Each nucleoside followed by a subscript “d” is a β-D-2′-deoxyribonucleoside. Nucleosides followed by a subscript “e” indicates a 2′-O-methoxyethyl (MOE) modified nucleoside. Nucleosides followed by a subscript “k” indicates a 6′-(S)—CH₃ bicyclic nucleoside (e.g. cEt). “N” indicates modified or naturally occurring nucleobases (A, T, C, G, U, or 5-methyl C).

### Treatment

Six week old BALB/C mice (purchased from Charles River) were injected subcutaneously twice a week for three weeks at dosage 10 mg/kg or 20 mg/kg with the modified oligonucleotides shown below or with saline control. Each treatment group consisted of 3 animals. The mice were sacrificed 48 hrs following last administration, and organs and plasma were harvested for further analysis.

mRNA Analysis

Liver tissues were homogenized and mRNA levels were quantitated using real-time PCR and normalized to RIBOGREEN as described herein. The results below are listed as Target-Y mRNA expression for each treatment group relative to saline-treated control (% UTC). As illustrated in Table 131, reduction in Target-Y mRNA expression levels was achieved with the oligonucleotides comprising two methyl phosphonate internucleoside linkages at the 5′-end of the gap region, ISIS 582071, 582072, 582069 and 582070.

### Plasma Chemistry Markers

Plasma chemistry markers such as liver transaminase levels, alanine aminotranferase (ALT) in serum were measured relative to saline treated mice and the results are presented in Table 131. Treatment with the oligonucleotides resulted in reduction in ALT level compared to treatment with the parent gapmer, ISIS 464917, 465178, 465984 or 466456. The results suggest that introduction of methyl phosphonate internucleoside linkage(s) can be useful for reduction of hepatoxicity profile of otherwise unmodified parent gapmers.

### Body and Organ Weights

Body weights, as well as liver, kidney and spleen weights were measured at the end of the study. The results in Table 132 are presented as the average percent of body and organ weights for each treatment group relative to saline-treated control. As illustrated, treatment with ISIS 582070 resulted in a reduction in liver and spleen weights compared to treatment with the parent gapmer, ISIS 466456. An increase in body and organ weights was observed for ISIS 582071 as compared to ISIS 464917. The remaining oligonucleotides, ISIS 582072 and 582069 did not cause any changes in body and organ weights outside the expected range as compared to ISIS 465178 and 465984.

TABLE 130
Modified oligonucleotides comprising methyl phosphonate internu-
cleoside linkages

| | | | | Wing | | SEQ |
|---|---|---|---|---|---|---|
| ISIS | | | Gap | Chemistry | | ID |
| NO | Sequence (5′ to 3′) | Motif | Chemistry | 5′ | 3′ | NO. |
| 464917 | NₖNₖNₖN_{d}N_{d}N_{d}N_{d}N_{d}N_{d}N_{d}N_{d}N_{d}N_{d}NₖNₖNₖ | 3-10-3 | Full deoxy | kkk | kkk | 6 |
| 582071 | NₖNₖNₖN_{dx}N_{dx}N_{d}N_{d}N_{d}N_{d}N_{d}N_{d}N_{d}N_{d}NₖNₖNₖ | 3-10-3 | Deoxy/Methyl | kkk | kkk | |
| | | | phosphonate | | | |
| 465178 | NₖNₖNₖN_{d}N_{d}N_{d}N_{d}N_{d}N_{d}N_{d}N_{d}N_{d}N_{d}NₖNₖNₖ | 3-10-3 | Full deoxy | kkk | kkk | 6 |
| 582072 | NₖNₖNₖN_{dx}N_{dx}N_{d}N_{d}N_{d}N_{d}N_{d}N_{d}N_{d}N_{d}NₖNₖNₖ | 3-10-3 | Deoxy/Methyl | kkk | kkk | |
| | | | phosphonate | | | |
| 465984 | NₖNₖNₖN_{d}N_{d}N_{d}N_{d}N_{d}N_{d}N_{d}N_{d}N_{d}N_{d}NₑNₑNₑ | 3-10-3 | Full deoxy | kkk | eee | 6 |
| 582069 | NₖNₖNₖN_{dx}N_{dx}N_{d}N_{d}N_{d}N_{d}N_{d}N_{d}N_{d}N_{d}NₖNₖNₖ | 3-10-3 | Deoxy/Methyl | kkk | kkk | |
| | | | phosphonate | | | |
| 466456 | NₖN_{d}NₖN_{d}NₖN_{d}N_{d}N_{d}N_{d}N_{d}N_{d}N_{d}N_{d}N_{d}NₑNₑ | 5-9-2 or | Full deoxy or | kdkdk | ee | 6 |
| | | 3-11-2 | deoxy/cEt | or kdk | | |
| 582070 | NₖN_{d}NₖN_{dx}N_{dx}N_{d}N_{d}N_{d}N_{d}N_{d}N_{d}N_{d}N_{d}N_{d}NₑNₑ | 3-11-2 | Deoxy/Methyl | kdk | ee | |
| | | | phosphonate | | | |

e = 2′-MOE, k = cEt, d = 2′-deoxyribonucleoside

TABLE 131
Effect of modified oligonucleotide treatment on Target-Y
reduction and liver function in BALB/C mice

| ISIS | Dosage | % | ALT | | Gap | Wing Chemistry | |
|---|---|---|---|---|---|---|---|
| NO. | (mg/kg/wk) | UTC | (IU/L) | Motif | Chemistry | 5′ | 3′ |
| Saline | 0 | 100 | 30 | — | — | — | — |
| 464917 | 10 | 29 | 1244 | 3-10-3 | Full deoxy | kkk | kkk |
| 464917 | 20 | 30.1 | 2335 | | | | |
| 582071 | 20 | 10.2 | 274 | 3-10-3 | Deoxy/Methyl | kkk | kkk |
| | | | | | phosphonate | | |
| 465178 | 10 | 4.9 | 1231 | 3-10-3 | Full deoxy | kkk | kkk |
| 465178 | 20 | 10.6 | 6731 | | | | |
| 582072 | 10 | 36.7 | 44.7 | 3-10-3 | Deoxy/Methyl | kkk | kkk |
| 582072 | 20 | 23.6 | 43.7 | | phosphonate | | |
| 465984 | 10 | 4.7 | 61 | 3-10-3 | Full deoxy | kkk | eee |
| 465984 | 20 | 0.9 | 57 | | | | |
| 582069 | 10 | 11.1 | 39.7 | 3-10-3 | Deoxy/Methyl | kkk | kkk |
| 582069 | 20 | 3.3 | 27.7 | | phosphonate | | |
| 466456 | 10 | 9.5 | 692 | 5-9-2 or | Full deoxy or | kdkdk | ee |
| 466456 | 20 | 10.5 | 2209 | 3-11-2 | deoxy/cEt | or kdk | |
| 582070 | 10 | 73.9 | 24 | 3-11-2 | Deoxy/Methyl | kdk | ee |
| 582070 | 20 | 51.3 | 36.7 | | phosphonate | | |

e = 2′-MOE, k = cEt, d = 2′-deoxyribonucleoside

TABLE 132
Effect of modified oligonucleotide treatment
on body and organ weights in BALB/C mice

| | | Body | Liver/ | Spleen/ | Kidney/ |
|---|---|---|---|---|---|
| | Dosage | wt rel to | Body | Body | Body |
| ISIS NO. | (mg/kg/wk) | predose (%) | Wt (%) | Wt (%) | Wt (%) |
| Saline | 0 | 108 | 100 | 100 | 100 |
| 464917 | 10 | 92.9 | 125 | 106.2 | 102.3 |
| 464917 | 20 | 71.1 | 110.9 | 67.2 | 107.3 |
| 582071 | 20 | 104.6 | 135.2 | 142.8 | 89.8 |
| 465178 | 10 | 94.9 | 131.3 | 108.1 | 85.3 |
| 465178 | 20 | 79.5 | 147.5 | 112 | 95.3 |
| 582072 | 10 | 109.2 | 117.3 | 111.7 | 104.8 |
| 582072 | 20 | 107.1 | 130.1 | 107.2 | 99.8 |
| 465984 | 10 | 111.4 | 117.6 | 110.1 | 98.8 |
| 465984 | 20 | 111.3 | 122.6 | 134.5 | 96.1 |
| 582069 | 10 | 107.8 | 106.2 | 97 | 100.6 |
| 582069 | 20 | 105.4 | 115.8 | 106.2 | 100.4 |
| 466456 | 10 | 109.7 | 148.6 | 198.7 | 105.9 |
| 466456 | 20 | 101.2 | 182.3 | 213.7 | 101.9 |
| 582070 | 10 | 111.2 | 100.3 | 116.7 | 100.8 |
| 582070 | 20 | 111.1 | 108.9 | 115.6 | 95.7 |

### Example 85

### Short-Gap Chimeric Oligonucleotides Targeting Target-Y

A series of chimeric antisense oligonucleotides was designed based on ISIS 464917 or 465178, wherein the central gap region contains ten 2′-deoxyribonucleosides. These gapmers were designed by introducing 2′-MOE modified nucleoside(s) at the wing(s) and/or shortening the central gap region to nine, eight, or seven 2′-deoxyribonucleosides.

The gapmers and their motifs are described in Table 133. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e” or “k” are sugar modified nucleosides. A subscript “e” indicates a 2′-O-methoxyethyl (MOE) modified nucleoside and a subscript “k” indicates a 6′-(S)—CH₃ bicyclic nucleoside (e.g. cEt).

TABLE 133
Short-gap antisense oligonucleotides
targeting Target-Y

| | | | SEQ |
|---|---|---|---|
| ISIS | | | ID |
| NO | Sequence (5′ to 3′) | Motif | NO. |
| 464917 | NₖNₖNₖNNNNNNNNNNNₖNₖNₖ | 3-10-3 | 6 |
| | | (kkk-d10-kkk) | |
| 465977 | NₖNₖNₖNNNNNNNNNNNₑNₑNₑ | 3-10-3 | 6 |
| | | (kkk-d10-eee) | |
| 573331 | NₑNₖNₖNNNNNNNNNNNₖNₖNₑ | 3-10-3 | 6 |
| | | (ekk-d10-kke) | |
| 573332 | NₑNₑNₖNₖNNNNNNNNNNₖNₖNₑ | 4-9-3 | 6 |
| | | (eekk-d9-kke) | |
| 573333 | NₑNₑNₑNₖNₖNNNNNNNNNₖNₖNₑ | 5-8-3 | 6 |
| | | (eeekk-d8-kke) | |
| 573334 | NₑNₑNₑNₑNₖNₖNNNNNNNNₖNₖNₑ | 6-7-3 | 6 |
| | | (eeeekk-d7-kke) | |
| 573335 | NₑNₖNₖNNNNNNNNNNₖNₖNₑNₑ | 3-9-4 | 6 |
| | | (ekk-d9-kkee) | |
| 573336 | NₑNₖNₖNNNNNNNNNₖNₖNₑNₑNₑ | 3-8-5 | 6 |
| | | (ekk-d8-kkeee) | |
| 573361 | NₑNₖNₖNNNNNNNNₖNₖNₑNₑNₑNₑ | 3-7-6 | 6 |
| | | (ekk-d7-kkeeee) | |
| 573338 | NₑNₑNₖNₖNNNNNNNNNₖNₖNₑNₑ | 4-8-4 | 6 |
| | | (eekk-d8-kkee) | |
| 573339 | NₑNₑNₑNₖNₖNNNNNNNNₖNₖNₑNₑ | 5-7-4 | 6 |
| | | (eeekk-d7-kkee) | |
| 573340 | NₑNₑNₖNₖNNNNNNNNₖNₖNₑNₑNₑ | 4-7-5 | 6 |
| | | (eekk-d7-kkeee) | |
| 573779 | NₖNₖNₖNNNNNNNNNₖNₑNₑNₑNₑ | 3-8-5 | 6 |
| | | (kkk-d8-keeee) | |
| 573780 | NₖNₖNₖNNNNNNNNNₖNₑNₑNₑNₑ | 3-8-5 | 6 |
| | | (kkk-d8-keeee) | |
| 573806 | NₖNₖNₖNNNNNNNNNₖNₑNₑNₑNₑ | 3-8-5 | 6 |
| | | (kkk-d8-keeee) | |
| 573782 | NₖNₖNₖNNNNNNNNNₖNₑNₑNₑNₑ | 3-8-5 | 6 |
| | | (kkk-d8-keeee) | |
| 573783 | NₖNₖNₖNNNNNNNNNₖNₑNₑNₑNₑ | 3-8-5 | 6 |
| | | (kkk-d8-keeee) | |
| 573784 | NₖNₖNₖNNNNNNNNNₖNₑNₑNₑNₑ | 3-8-5 | 6 |
| | | (kkk-d8-keeee) | |
| 573785 | NₖNₖNₖNNNNNNNNNₖNₑNₑNₑNₑ | 3-8-5 | 6 |
| | | (kkk-d8-keeee) | |
| 573786 | NₖNₖNₖNNNNNNNNNₖNₑNₑNₑNₑ | 3-8-5 | 6 |
| | | (kkk-d8-keeee) | |
| 573787 | NₖNₖNₖNNNNNNNNNₖNₑNₑNₑNₑ | 3-8-5 | 6 |
| | | (kkk-d8-keeee) | |
| 465178 | NₖNₖNₖNNNNNNNNNNNₖNₖNₖ | 3-10-3 | 6 |
| | | (kkk-d10-kkk) | |
| 466140 | NₖNₖNₖNNNNNNNNNNNₑNₑNₑ | 3-10-3 | 6 |
| | | (kkk-d10-eee) | |
| 573341 | NₑNₖNₖNNNNNNNNNNNₖNₖNₑ | 3-10-3 | 6 |
| | | (ekk-d10-kke) | |
| 573342 | NₑNₑNₖNₖNNNNNNNNNNₖNₖNₑ | 4-9-3 | 6 |
| | | (eekk-d9-kke) | |
| 573343 | NₑNₑNₑNₖNₖNNNNNNNNNₖNₖNₑ | 5-8-3 | 6 |
| | | (eeekk-d8-kke) | |
| 573344 | NₑNₑNₑNₑNₖNₖNNNNNNNNₖNₖNₑ | 6-7-3 | 6 |
| | | (eeeekk-d7-kke) | |
| 573345 | NₑNₖNₖNNNNNNNNNNₖNₖNₑNₑ | 3-9-4 | 6 |
| | | (ekk-d9-kkee) | |
| 573346 | NₑNₖNₖNNNNNNNNNₖNₖNₑNₑNₑ | 3-8-5 | 6 |
| | | (ekk-d8-kkeee) | |
| 573347 | NₑNₖNₖNNNNNNNNₖNₖNₑNₑNₑNₑ | 3-7-6 | 6 |
| | | (ekk-d7-kkeeee) | |
| 573348 | NₑNₑNₖNₖNNNNNNNNNₖNₖNₑNₑ | 4-8-4 | 6 |
| | | (eekk-d8-kkee) | |
| 573349 | NₑNₑNₑNₖNₖNNNNNNNNₖNₖNₑNₑ | 5-7-4 | 6 |
| | | (eeekk-d7-kkee) | |
| 573350 | NₑNₑNₖNₖNNNNNNNNₖNₖNₑNₑNₑ | 4-7-5 | 6 |
| | | (eekk-d7-kkeee) | |
| 573788 | NₖNₖNₖNNNNNNNNNₖNₑNₑNₑNₑ | 3-8-5 | 6 |
| | | (kkk-d8-keeee) | |
| 573789 | NₖNₖNₖNNNNNNNNNₖNₑNₑNₑNₑ | 3-8-5 | 6 |
| | | (kkk-d8-keeee) | |
| 573790 | NₖNₖNₖNNNNNNNNNₖNₑNₑNₑNₑ | 3-8-5 | 6 |
| | | (kkk-d8-keeee) | |
| 573791 | NₖNₖNₖNNNNNNNNNₖNₑNₑNₑNₑ | 3-8-5 | 6 |
| | | (kkk-d8-keeee) | |
| 573792 | NₖNₖNₖNNNNNNNNNₖNₑNₑNₑNₑ | 3-8-5 | 6 |
| | | (kkk-d8-keeee) | |
| 573793 | NₖNₖNₖNNNNNNNNNₖNₑNₑNₑNₑ | 3-8-5 | 6 |
| | | (kkk-d8-keeee) | |
| 573794 | NₖNₖNₖNNNNNNNNNₖNₑNₑNₑNₑ | 3-8-5 | 6 |
| | | (kkk-d8-keeee) | |
| 573795 | NₖNₖNₖNNNNNNNNNₖNₑNₑNₑNₑ | 3-8-5 | 6 |
| | | (kkk-d8-keeee) | |
| 573796 | NₖNₖNₖNNNNNNNNNₖNₑNₑNₑNₑ | 3-8-5 | 6 |
| | | (kkk-d8-keeee) | |
| 141923 | CₑCₑTₑTₑCₑCCTGAAGGTTCₑCₑTₑCₑCₑ | 5-10-5 | 9 |
| (neg control) | | (e5-d10-e5) | |

e = 2′-MOE (e.g. e5 = eeeee), k = cEt, d = 2′-deoxyribonucleoside

### Example 86

### Short-Gap Chimeric Oligonucleotides Targeting Target-V—In Vitro Study

Several short-gap chimeric oligonucleotides from Table 133 were selected and evaluated for their effects on Target-Y mRNA in vitro. The parent gapmer, ISIS 464917 and 465178 were included in the study for comparison. ISIS 141923 was used as a negative control.

The newly designed gapmers were tested in vitro. Primary mouse hepatocytes at a density of 35,000 cells per well were transfected using electroporation with 0.0625, 0.25, 1, 4 and 16 μM concentrations of chimeric oligonucleotides. After a treatment period of approximately 24 hours, RNA was isolated from the cells and Target-Y mRNA levels were measured by quantitative real-time PCR. Primer probe set RTSXXXX was used to measure mRNA levels. Target-Y mRNA levels were adjusted according to total RNA content, as measured by RIBOGREEN.

The half maximal inhibitory concentration (IC₅₀) of each oligonucleotide is presented in Table 134 and was calculated by plotting the concentrations of oligonucleotides used versus the percent inhibition of Target-Y mRNA expression achieved at each concentration, and noting the concentration of oligonucleotide at which 50% inhibition of Target-Y mRNA expression was achieved compared to the control. As illustrated in Table 134 and 135, several short-gap oligonucleotides showed comparable inhibition of Target-Y mRNA levels as compared to the parent gapmers, ISIS 464917 or 465178.

TABLE 134
Comparison of inhibition of Target-Y mRNA levels
of short-gap oligonucleotides with ISIS 464917

| | | IC₅₀ | SEQ ID |
|---|---|---|---|
| ISIS NO | Motif | (μM) | NO. |
| 464917 | 3-10-3 | 0.5 | 6 |
| | (kkk-d10-kkk) | | |
| 573331 | 3-10-3 | 0.5 | 6 |
| | (ekk-d10-kke) | | |
| 573332 | 4-9-3 | 0.6 | 6 |
| | (eekk-d9-kke) | | |
| 573333 | 5-8-3 | 0.5 | 6 |
| | (eeekk-d8-kke) | | |
| 573335 | 3-9-4 | 0.4 | 6 |
| | (ekk-d9-kkee) | | |
| 573336 | 3-8-5 | 0.5 | 6 |
| | (ekk-d8-kkeee) | | |
| 573361 | 3-7-6 | 0.6 | 6 |
| | (ekk-d7-kkeeee) | | |
| 573340 | 4-7-5 | 2.3 | 6 |
| | (eekk-d7-kkeee) | | |
| 141923 | 5-10-5 | >16 | 9 |
| (neg control) | (e5-d10-e5) | | |

e = 2′-MOE (e.g. e5 = eeeee), k = cEt, d = 2′-deoxyribonucleoside

TABLE 135
Comparison of inhibition of Target-Y mRNA levels
of short-gap oligonucleotides with ISIS 465178

| | | IC₅₀ | SEQ |
|---|---|---|---|
| ISIS NO | Motif | (μM) | ID NO. |
| 465178 | 3-10-3 | 0.2 | 6 |
| | (kkk-d10-kkk) | | |
| 573341 | 3-10-3 | 0.2 | 6 |
| | (ekk-d10-kke) | | |
| 573342 | 4-9-3 | 0.4 | 6 |
| | (eekk-d9-kke) | | |
| 573345 | 3-9-4 | 0.2 | 6 |
| | (ekk-d9-kkee) | | |
| 573346 | 3-8-5 | 0.4 | 6 |
| 573348 | (ekk-d8-kkeee) | 0.5 | 6 |
| 573350 | 4-8-4 | 0.9 | 6 |
| | (eekk-d8-kkee) | | |
| 573806 | 4-7-5 | 0.8 | 6 |
| | (eekk-d7-kkeee) | | |
| 573783 | 3-8-5 | 1.0 | 6 |
| | (kkk-d8-keeee) | | |
| 573784 | 3-8-5 | 1.3 | 6 |
| | (kkk-d8-keeee) | | |
| 573785 | 3-8-5 | 1.0 | 6 |
| | (kkk-8-keeee) | | |
| 573792 | 3-8-5 | 0.5 | 6 |
| | (kkk-8-keeee) | | |
| 573794 | 3-8-5 | 0.4 | 6 |
| | (kkk-d8-keeee) | | |
| 573795 | 3-8-5 | 0.5 | 6 |
| | (kkk-d8-keeee) | | |
| 573796 | 3-8-5 | 0.8 | 6 |
| | (kkk-d8-keeee) | | |
| 141923 | 5-10-5 | >16 | 6 |
| (neg control) | (e5-d10-e5) | | |

e = 2′-MOE (e.g. e5 = eeeee), k = cEt, d = 2′-deoxyribonucleoside

### Example 87

### Short-Gap Chimeric Oligonucleotides Targeting Target-Y—In Vivo Study

Several short-gap oligonucleotides described in Example 85 were selected and evaluated for efficacy in vivo and for changes in the levels of various plasma chemistry markers targeting Target-Y. The parent gapmer, ISIS 464917 was included in the study for comparison.

### Treatment

Six week male BALB/C mice (purchased from Charles River) were injected subcutaneously with a single dose of antisense oligonucleotide at 10 mg/kg or 20 mg/kg or with saline control. Each treatment group consisted of 4 animals. The mice were sacrificed 96 hrs following last administration, and organs and plasma were harvested for further analysis.

mRNA Analysis

Liver tissues were homogenized and mRNA levels were quantitated using real-time PCR and normalized to Cyclophilin A as described herein. The results below are listed as Target-Y mRNA expression for each treatment group relative to saline-injected control (% UTC). As illustrated in Table 136, Target-Y mRNA expression levels were reduced in a dose-dependent manner with the newly designed oligonucleotides.

### Plasma Chemistry Markers

Plasma chemistry markers such as liver transaminase levels, alanine aminotranferase (ALT) in serum were measured relative to saline treated mice and the results are presented in Table 136. Treatment with the newly designed oligonucleotides resulted in reduction in ALT levels compared to treatment with the parent gapmer, ISIS 464917. The results suggest that shortening the central gap region and introducing 2′-MOE modified nucleoside(s) at the wing(s) can be useful for the reduction of hepatoxicity profile of ISIS 464917.

### Body and Organ Weights

Body weights, as well as liver, kidney and spleen weights were also measured at the end of the study. The results showed that treatment with the newly designed oligonucleotides did not cause any changes in body and organ weights outside the expected range as compared to ISIS 464917 (data not shown).

TABLE 136
Effect of short-gap antisense oligonucleotide treatment
on Target-Y reduction and liver function in BALB/C mice

| | Dosage | % | ALT | | SEQ |
|---|---|---|---|---|---|
| ISIS NO | (mg/kg/wk) | UTC | (IU/L) | Motif | ID NO. |
| Saline | 0 | 99 | 23 | — | |
| 464917 | 10 | 11.5 | 1834 | 3-10-3 | 6 |
| | 20 | 5.1 | 8670 | (kkk-d10-kkk) | |
| 573333 | 10 | 32.8 | 79 | 5-8-3 | 6 |
| | 20 | 21.2 | 370 | (eeekk-d8-kke) | |
| 573334 | 10 | 79.5 | 26 | 6-7-3 | 6 |
| | 20 | 69.4 | 29 | (eeeekk-d7-kke) | |
| 573336 | 10 | 23.2 | 179 | 3-8-5 | 6 |
| | 20 | 12.0 | 322 | (ekk-d8-kkeee) | |
| 573339 | 10 | 47.9 | 35 | 5-7-4 | 6 |
| | 20 | 32.8 | 199 | (eeekk-d7-kkee) | |
| 573340 | 10 | 81.3 | 63 | 4-7-5 | 6 |
| | 20 | 66.2 | 33 | (eekk-d7-kkeee) | |
| 573361 | 10 | 33.6 | 150 | 3-7-6 | 6 |
| | 20 | 19.2 | 722 | (ekk-d7-kkeeee) | |
| 573783 | 10 | 16.5 | 734 | 3-8-5 | 6 |
| | 20 | 6.3 | 1774 | (kkk-d8-keeee) | |
| 573785 | 10 | 20.2 | 61 | 3-8-5 | 6 |
| | 20 | 14.2 | 40 | (kkk-d8-keeee) | |
| 573806 | 10 | 19.3 | 346 | 3-8-5 | 6 |
| | 20 | 15.4 | 1389 | (kkk-d8-keeee) | |

e = 2′-MOE, k = cEt, d = 2′-deoxyribonucleoside

### Example 88

### Short-Gap Chimeric Oligonucleotides Targeting PTEN

A series of chimeric antisense oligonucleotides was designed based on ISIS 482050, wherein the central gap region contains ten 2′-deoxyribonucleosides. These gapmers were designed by introducing 2′-MOE modified nucleoside(s) at the wing(s) and/or shortening the central gap region to nine, or eight 2′-deoxyribonucleosides.

The gapmers and their motifs are described in Table 137. The internucleoside linkages throughout each gapmer are phosphorothioate (P═S) linkages. All cytosine nucleobases throughout each gapmer are 5-methyl cytosines. Nucleosides without a subscript are β-D-2′-deoxyribonucleosides. Nucleosides followed by a subscript “e” or “k” are sugar modified nucleosides. A subscript “e” indicates a 2′-O-methoxyethyl (MOE) modified nucleoside and a subscript “k” indicates a 6′-(S)—CH₃ bicyclic nucleoside (e.g. cEt).

TABLE 137
Short-gap antisense oligonucleotides
targeting PTEN

| | | | SEQ |
|---|---|---|---|
| ISIS | | | ID |
| NO. | Sequence (5′ to 3′) | Motif | NO. |
| 482050 | AₖTₖCₖATGGCTGCAGCₖTₖTₖ | 3-10-3 | 85 |
| | | (kkk-d10-kkk) | |
| 508033 | AₖTₖCₖATGGCTGCAGCₑTₑTₑ | 3-10-3 | 85 |
| | | (kkk-d10-eee) | |
| 573351 | AₑTₖCₖATGGCTGCAGCₖTₖTₑ | 3-10-3 | 85 |
| | | (ekk-d10-kke) | |
| 573352 | AₑTₑCₖAₖTGGCTGCAGCₖTₖTₑ | 4-9-3 | 85 |
| | | (eekk-d9-kke) | |
| 573353 | AₑTₑCₑAₖTₖGGCTGCAGCₖTₖTₑ | 5-8-3 | 85 |
| | | (eeekk-d8-kke) | |
| 573354 | AₑTₑCₑAₑTₖGₖGCTGCAGCₖTₖTₑ | 6-7-3 | 85 |
| | | (eeeekk-d7-kke) | |
| 573355 | AₑTₖCₖATGGCTGCAGₖCₖTₑTₑ | 3-9-4 | 85 |
| | | (ekk-d9-kkee) | |
| 573356 | AₑTₖCₖATGGCTGCAₖGₖCₑTₑTₑ | 3-8-5 | 85 |
| | | (ekk-d8-kkeee) | |
| 573357 | AₖTₖCₖATGGCTGCₖAₖGₑCₑTₑTₑ | 3-7-6 | 85 |
| | | (ekk-d7-kkeeee) | |
| 573358 | AₑTₑCₖAₖTGGCTGCAGₖCₖTₑTₑ | 4-8-4 | 85 |
| | | (eekk-d8-kkee) | |
| 573359 | AₑTₑCₑAₖTₖGGCTGCAGₖCₖTₑTₑ | 5-7-4 | 85 |
| | | (eeekk-d7-kkee) | |
| 573360 | AₑTₑCₖAₖTGGCTGCAₖGₖCₑTₑTₑ | 4-7-5 | 85 |
| | | (eekk-d7-kkeee) | |
| 573797 | TₖGₖGₖCTGCAGCTTₖCₑCₑGₑAₑ | 3-8-5 | 87 |
| | | (kkk-d8-keeee) | |
| 573798 | AₖTₖGₖGCTGCAGCTₖTₑCₑCₑGₑ | 3-8-5 | 88 |
| | | (kkk-d8-keeee) | |
| 573799 | CₖAₖTₖGGCTGCAGCₖTₑTₑCₑCₑ | 3-8-5 | 89 |
| | | (kkk-d8-keeee) | |
| 573800 | TₖCₖAₖTGGCTGCAGₖCₑTₑTₑCₑ | 3-8-5 | 90 |
| | | (kkk-d8-keeee) | |
| 573801 | AₖTₖCₖATGGCTGCAₖGₑCₑTₑTₑ | 3-8-5 | 85 |
| | | (kkk-d8-keeee) | |
| 573802 | CₖAₖTₖCATGGCTGCₖAₑGₑCₑTₑ | 3-8-5 | 91 |
| | | (kkk-d8-keeee) | |
| 573803 | CₖCₖAₖTCATGGCTGₖCₑAₑGₑCₑ | 3-8-5 | 92 |
| | | (kkk-d8-keeee) | |
| 573804 | TₖCₖCₖATCATGGCTₖGₑCₑAₑGₑ | 3-8-5 | 93 |
| | | (kkk-d8-keeee) | |
| 573805 | TₖTₖCₖCATCATGGCₖTₑGₑCₑAₑ | 3-8-5 | 94 |
| | | (kkk-d8-keeee) | |

e = 2′-MOE, k = cEt, d = 2′-deoxyribonucleoside

### Example 89

### Short-Gap Chimeric Oligonucleotides Targeting PTEN—In Vitro Study

Several short-gap chimeric oligonucleotides from Table 137 were selected and evaluated for their effects on PTEN mRNA in vitro. The parent gapmer, ISIS 482050 were included in the study for comparison. ISIS 141923 was used as a negative control.

The newly designed gapmers were tested in vitro. Primary mouse hepatocytes at a density of 35,000 cells per well were transfected using electroporation with 0.0625, 0.25, 1, 4 and 16 μM concentrations of chimeric oligonucleotides. After a treatment period of approximately 24 hours, RNA was isolated from the cells and PTEN mRNA levels were measured by quantitative real-time PCR. Primer probe set RTS186 was used to measure mRNA levels. PTEN mRNA levels were adjusted according to total RNA content, as measured by RIBOGREEN.

The half maximal inhibitory concentration (IC₅₀) of each oligonucleotide was calculated in the same manner as described previously and the results are presented in Table 138. As illustrated, most short-gap oligonucleotides showed comparable inhibition of PTEN mRNA levels as compared to ISIS 482050.

TABLE 138
Comparison of inhibition of PTEN mRNA levels of
short-gap oligonucleotides with ISIS 482050

| | | IC₅₀ | SEQ |
|---|---|---|---|
| ISIS NO | Motif | (μM) | ID NO. |
| 482050 | 3-10-3 | 1.9 | 85 |
| | (kkk-d10-kkk) | | |
| 573351 | 3-10-3 | 2.8 | 85 |
| 573353 | (ekk-d10-kke) | 6.1 | 85 |
| 573355 | 3-9-4 | 2.6 | 85 |
| | (ekk-d9-kkee) | | |
| 573798 | 3-8-5 | 1.6 | 88 |
| | (kkk-d8-keeee) | | |
| 573799 | 3-8-5 | 1.9 | 89 |
| | (kkk-d8-keeee) | | |
| 573803 | 3-8-5 | 1.4 | 92 |
| | (kkk-d8-keeee) | | |
| 141923 | 5-10-5 | >16 | 9 |
| (neg control) | (e5-d10-e5) | | |

e = 2′-MOE (e.g. e5 = eeeee), k = cEt, d = 2′-deoxyribonucleoside

1.-272. (canceled)

273. A oligomeric compound comprising a modified oligonucleotide consisting of 10 to 30 linked nucleosides, wherein the modified oligonucleotide has a modification motif comprising:
a 5′-region consisting of 2-8 linked 5′-region nucleosides, each independently selected from among a modified nucleoside and an unmodified deoxynucleoside, provided that at least one 5′-region nucleoside is a modified nucleoside and wherein the 3′-most 5′-region nucleoside is a modified nucleoside;
a 3′-region consisting of 2-8 linked 3′-region nucleosides, each independently selected from among a modified nucleoside and an unmodified deoxynucleoside, provided that at least one 3′-region nucleoside is a modified nucleoside and wherein the 5′-most 3′-region nucleoside is a modified nucleoside; and
a central region between the 5′-region and the 3′-region consisting of 6-12 linked central region nucleosides, wherein each central region nucleoside is an unmodified deoxynucleoside;
wherein the modified oligonucleotide has a nucleobase sequence complementary to the nucleobase sequence of a target region of a target nucleic acid.

274. The oligomeric compound of claim 273, wherein the 3′-most 5′-region nucleoside comprises a bicyclic sugar moiety.

275. The oligomeric compound of claim 274, wherein the 3′-most 5′-region nucleoside comprises a cEt sugar moiety or an LNA sugar moiety.

276. The oligomeric compound of claim 273, wherein at least one 5′-region nucleoside is an unmodified deoxynucleoside.

277. The oligomeric compound of claim 273, wherein each 5′-region nucleoside is a modified nucleoside.

278. The oligomeric compound of claim 273, comprising at least one modified 5′-region nucleoside comprising a modified sugar.

279. The oligomeric compound of claim 273, wherein at least one 3′-region nucleoside is an unmodified deoxynucleoside.

280. The oligomeric compound of claim 273, wherein each 3′-region nucleoside is a modified nucleoside.

281. The oligomeric compound of claim 273, comprising at least one modified 3′-region nucleoside comprising a modified sugar.

282. A oligomeric compound comprising a modified oligonucleotide consisting of 10 to 30 linked nucleosides, wherein the modified oligonucleotide has a modification motif comprising:
a 5′-region consisting of 2-8 linked 5′-region nucleosides, each independently selected from among a modified nucleoside and an unmodified deoxynucleoside, provided that at least one 5′-region nucleoside is a modified nucleoside and wherein the 3′-most 5′-region nucleoside is a modified nucleoside;
a 3′-region consisting of 2-8 linked 3′-region nucleosides, each independently selected from among a modified nucleoside and an unmodified deoxynucleoside, provided that at least one 3′-region nucleoside is a modified nucleoside and wherein the 5′-most 3′-region nucleoside is a modified nucleoside; and
a central region between the 5′-region and the 3′-region consisting of 6-12 linked central region nucleosides, each independently selected from among: a modified nucleoside and an unmodified deoxynucleoside, wherein at least one central region nucleoside is a modified nucleoside comprising a modified sugar moiety, and wherein the 5′-most central region nucleoside is an unmodified deoxynucleoside and the 3′-most central region nucleoside is an unmodified deoxynucleoside;
wherein the modified oligonucleotide has a nucleobase sequence complementary to the nucleobase sequence of a target region of a target nucleic acid.

283. The oligomeric compound of claim 282, wherein the at least one modified central region nucleoside comprises a 5′-methyl-2′-deoxy sugar moiety.

284. The oligomeric compound of claim 282, wherein the at least one modified central region nucleoside comprises a bicyclic sugar moiety.

285. The oligomeric compound of claim 282, wherein the at least one modified central region nucleoside comprises a cEt sugar moiety, an LNA sugar moiety, or an α-LNA sugar moiety.

286. The oligomeric compound of claim 282, wherein the at least one modified central region nucleoside comprises a 2′-substituted sugar moiety.

287. The oligomeric compound of claim 282, wherein the at least one modified central region nucleoside comprises a 2′-substituted sugar moiety comprising a 2′ substituent selected from among: halogen, optionally substituted allyl, optionally substituted amino, azido, optionally substituted SH, CN, OCN, CF3, OCF3, O, S, or N(Rm)-alkyl; O, S, or N(Rm)-alkenyl; O, S or N(Rm)-alkynyl; optionally substituted O-alkylenyl-O-alkyl, optionally substituted alkynyl, optionally substituted alkaryl, optionally substituted aralkyl, optionally substituted O-alkaryl, optionally substituted O-aralkyl, O(CH2)2SCH3, O—(CH2)2-O—N(Rm)(Rn) or O-CH2-C(═O)—N(Rm)(Rn), where each Rm and Rn is, independently, H, an amino protecting group or substituted or unsubstituted C₁-C₁₀ alkyl; wherein each optionally substituted group is optionally substituted with a substituent group independently selected from among: hydroxyl, amino, alkoxy, carboxy, benzyl, phenyl, nitro (NO₂), thiol, thioalkoxy (S-alkyl), halogen, alkyl, aryl, alkenyl and alkynyl.

288. The oligomeric compound of claim 282, wherein the at least one modified central region nucleoside comprises a 2′-MOE, 2′-OMe, 2′-F, or 2′-(ara)-F sugar moiety.

289. The oligomeric compound of claim 282, wherein the at least one modified central region nucleoside comprises a sugar surrogate.

290. The oligomeric compound of claim 282, comprising at least one modified central region nucleoside comprising a modified nucleobase.

291. A method of ameliorating a symptom of Alzheimer's disease, Creutzfeldt-Jakob disease, fatal familial insomnia, Alexander disease, Parkinson's disease, amyotrophic lateral sclerosis, dentato-rubral and pallido-luysian atrophy DRPA, spino-cerebellar ataxia, Torsion dystonia, cardiomyopathy, chronic obstructive pulmonary disease (COPD), liver disease, hepatocellular carcinoma, systemic lupus erythematosus, hypercholesterolemia, breast cancer, asthma, Type 1 diabetes, Rheumatoid arthritis, Graves disease, SLE, spinal and bulbar muscular atrophy, Kennedy's disease, progressive childhood posterior subcapsular cataracts, cholesterol gallstone disease, arthrosclerosis, cardiovascular disease, primary hypercalciuria, alpha-thallasemia, obsessive compulsive disorder, Anxiety, comorbid depression, congenital visual defects, hypertension, metabolic syndrome, prostate cancer, congential myasthenic syndrome, peripheral arterial disease, atrial fibrillation, sporadic pheochromocytoma, congenital malformations, Machado-Joseph disease, Huntington's disease, and Autosomal Dominant Retinitis Pigmentosa disease, comprising administering an oligomeric compound to an animal in need thereof, wherein the oligomeric compound comprises a modified oligonucleotide consisting of 10 to 30 linked nucleosides, wherein the modified oligonucleotide has a modification motif comprising:
a 5′-region consisting of 2-8 linked 5′-region nucleosides, each independently selected from among a modified nucleoside and an unmodified deoxynucleoside, provided that at least one 5′-region nucleoside is a modified nucleoside and wherein the 3′-most 5′-region nucleoside is a modified nucleoside;
a 3′-region consisting of 2-8 linked 3′-region nucleosides, each independently selected from among a modified nucleoside and an unmodified deoxynucleoside, provided that at least one 3′-region nucleoside is a modified nucleoside and wherein the 5′-most 3′-region nucleoside is a modified nucleoside; and
a central region between the 5′-region and the 3′-region consisting of 6-12 linked central region nucleosides, each independently selected from among: a modified nucleoside and an unmodified deoxynucleoside, wherein at least one central region nucleoside is a modified nucleoside comprising a modified sugar moiety, and wherein the 5′-most central region nucleoside is an unmodified deoxynucleoside and the 3′-most central region nucleoside is an unmodified deoxynucleoside;
and wherein the modified oligonucleotide has a nucleobase sequence complementary to the nucleobase sequence of a target region of a target nucleic acid.

292. The method of claim 291, wherein the animal is a human.
